SEQUENCE LISTING (275 sequences)

SEQ ID NO: 1 - DNA, 147700 bp, Homo sapiens
  misc_feature (0)...(147700): n = a, c, g, or t
aaagccaagactcccattcctaaaccctagctcaggtctccatctcttaaatccgagtgacctctacaaactctccctgagaaggtgtccagaacccttttggaagcgagggacagtgtcactgtctttggggttgacacctgctctgagtaactcacggaaaacaagttccagctgggaagcccttggacgcgccacacctccctacccgcagcccgtcctgtggcgcccgggactccagagtgygtttagatgaaaggggaccgacacgtcagggccaccgcgggaagcgctgagggccactcaccggccagggacgcgaagagcgcggccgccgcgctgagctgccggggcatggtgggcgctgggcgaggttctgcagcgtgcggcgargtccgggcaggccccgaatcggtgccagagaaacctacctgtgccggagaaacgaaaccacctgcttatgagaagcagccgaaaagcccgcccagggccgctgggcggggagggaaactccgccggccccctcctacccctacggagcagggaggggcggggactcggcgcagccgccggggcccgggcctctgggaccgtttaccgcacgcgcgtggtcccggcagcgccggcstcctccgctcataccctgggtctcctcctttctttttcttttctttttgagacgaagtctcgctctgtcgcccagggtggagtgcagtggcgcgatctcggctcactacaacctctgcctcccgggttcaagcgattcttctgcctcagcctcccgagtagctgggattacaggcatgcaccaccacacccggctaatttttgtatttttagtagagacggggtgtcaccatattggccaggctggtctcgagctcctgacctcgtgattcgcccgcctcgacctcccaaagtgctgggattatagacgtgagccaccgagcccggccagggtctcctcttttatttcttttctttttatttcttttgttttgttttgttttgttttgttttttgagacaaagtctcgctctgtcgccaggctggagtgcagtggcgggatctcggctcactgccctggttcaagcgattctcctgccgcagcctcccgagtagctggggttacaggcgcccgccaccacgcccagctaatttttgtattttagtagagacggggtttcaccctgttggccaggctggtctcgatctcctgaccttgtgatccgcccgcctcggcctcccaaagtgttgggattacaggcgtgagccactgcgcccggcccagggtctcctcttttctaacagctcgggtacctttctgggaacccagagacgcttctcagccgggagaaagccagccactaggcgagcaggagcctaaaaacccctaagcaccctgactccatgtcttcccagggagtctgcggcagccgcgctccacgcccaggcctcgccaggaccgcggtttgcgggaagcaacaggagcacagcccagaggcgctaggtctggctgggagctcgcgctgccgactccccggcgtgcggcgtcggggaacctctaggagccttggattcttcagctgtaaaacggacataataatgcccactcccagtgtgtttttttattttcttttttctttttctttctttgtttttgtttgtttgtttttgtttttgtttttgagacagggtctcactctgtcgcccaggctggagggcaatggcgtgatctcggctcactgcaaacttgggttcaggcgattctcctgcctcagcctccacagtagctgggattacagatgtgcgccaccacgtccggctaattttttgtatttttagtagagaccaggtttcaccgtgttggccaagctggtctcaaactcctgaccccaggtgatccgcccgcctcggccttccaaagatctgggattacaagcgtgagccactgtgcctggccccaggtggttttacagaccagaaaatcctggaacaaaaaacacacaatatcgttttttttttttttttggagtcagggtctcgctctatcacccaggctggagtgcagtggcgtgatctcggctcactgcaacttcgacctcctggcctcaagtgagtctcccaccttagcctcctgagtagctgggaccaaaggcgcgtgccaccacgcccagctattttattttattttatgtagagaggaggtctcgctgtgttgcccaggctggtctcgagttcctggcctcaaatgatcctcctgcgttagccaaccattgggattacaggcgtaagccacggcccacggctcaacaacgctgacaggcaaccttttaatgtcttatctccttcctctattaattggattgtctgtcaaaacaacgatgttttgacagggcttgagtcccagtggggaatacacatttaagcagtatattaggagaccctccttatcactagattgagggctttcagcctagcctcaaattattttctgaaaaataactttggctacaactattttgtcttactatgttgctccaaacactaatcaagtaaacttaaccaaagcttgcagtgtgtttcagaatggaatttttatggtgaaaagtgagggttaacttgtgccagtcaacctagtttcagcaactacctgctttctgatctttgagacagtttattcaaaagacgataattaagtgggtatagactgtgtgccaggcactcttcttattccatttaagcgccatagccactctatatggacactgttgttattatcgctgccccatttcgcagatggagaaactaagcacaaagaagggagttgcccagagtcacttagataataaataccgaaacctgaccataaatcttgtctgccttgagagtctaggattttaagcacatagccgggcgcagtggctcacgcctgtaatcccagcactttaggaggccgaggcgggcggatcacgaggtcaggagatggagaccatcctggctaacacagtgaaacccagttctattaaaaatataaaaaaattagccaggcgtggtggcaggtgcatgtagtcccagctactcgggaggctgaggcaggagaatggtgtgaacccaggaggtggagcttgcagtgagcggagatcgcgccactgcactccagcccgggcgacagaaggagactccgtctcaaaaaaaaaaaaataaataaaataaagctgctcctcttaccctggaaattccaagggatttaggagctctgtttcaggaaccagggtcaaagaccaagtattaaaacaaaagattctcctagaactctggcatataaggattttaggagctctgtcttagaaactgggacagagaccaaagatatattattatatcgcagtatcatagtttattattttcaaaaaacgttttctggctggtacagtggttcatgcctataatccaagcactttgggaggccaaggtgggagggtcacttgaggccagaagttcaagtccagcctgggcaacacagggagaccctgccactattaaaaattttttaaattagctgggcatggtggcacatgcctgtagtcccagctacttgggaggctgaagcaggaggattgcttgagcctgagaggtcaagactgtagtgagctgcgatcaagcgactgcactctagcctgggtgacaaagccagaccctgtgtctaaaaaaaagaaaagaagaggaaaaaaaaaggtttttatttcaactaagttgttggatttattagcataaagcttttcataacaatcccttgtcttacaatatctgtagtataggtagtgatgtcacttcttttattactaatattaataatttgtattttctctcttatttccctgctaatattaataatttgtattttctctcttttttccctgataagtttggctggaggttgatcaattctattcatgttttcaagaaaaaaaaaaaaatttcatttcatcgagttccttcattgttcttatgttttctgttttattcatttctacctgatctttattattttctttcttctacttaacttgtgttttatttgctcctctttcaatagttttcaaagatggaacttgcctgggatatcccagcactttaggaggctgaggtgggaggatcacctgaggtcaggagttcaagaccagcctggccaacatggtgaaactccgtctctactaaaaatacaaaaattagctgggtgtggtggtgggcacctgtgatcccagctactcgggaggctgaggcaggagaatcgcttgaacccgggaggtggaggttacagtgagctgagatcacgccactgcactccagcctgggtgacaggagctagactctgtctcaaaaaaaaaaaaagaaaaaaaaaaagatggaagttgaggccagtggtataaaatctttcttcatctctaataaacaggtttactgttatgaacgtccctctaattactactttagttggatcccacaagtttaatatgttttcattttcatataattagaaagacttcctaatttccttttgctatctcctcgactcatggttatttaaaatagcattatttcatttccaaacatatagtttttcagatatctttcttttattgatttttaattccactgtggttgaaaacataattatggacttaaatcttacaaatttattgatatttgttttttgaccaagactatggttggttacattagttttcaggactgacataacaaagtgccacagactgggtaattaaaccacagacatttgctttcttataattctggaaaccagacatgtgagatcaaagtgtcagccgggttggttttttctttttccttttttttttttttttttgagacagagtctctttctgtcacccaggctgggatgcagtggtgtgatctcggcttactgcaaattctgcctcccaggctcaagcgattctcctgcctcagcctcccgagtagctgggattacaggtgcctctcactgcacctggctaatttttgtatttttagtagagacggggtttcaccatgttggccaggttgatctcaaactcctgatctcaggtaatacacccgcctcggcctcccaaagtactgagattacaggcgtgagccactgcacccggcccgggttagttctttctaaggcctctctccttggctagtagacacctttgtttcacatggtcatccctctgtgcatgcctttgtctgtcctaatctcctcttcttataaggacattagtcaggtaggattagtgcctactctttgaactcgttttacctcttaaagaccctatctccgaatatagtcacattctgagatacttggggttaagacttgtattagtccattttcacgctgctcataaagacatacctgagactgggaagaaaaagaggtttaattggacaattccacatggctggggaggcctcagaatcatggtgggaggcgaaagggactttttacatggtggcggcaagagaaaatgaggaagaagcaaaagcagaaacccctgatagataagcccaccagatatcatgagatttattcactgtcatgagaacagcacgggaaagaccagcccccatgaatacattacctcttccttggtccccccctccccacaatatgtggggattctgggagatacaattaaaattgagatttgagtggggacacagccaaaccatatcattctgtccctggtccttccaaatctcatgtcctcacatttcaaaaccaatcatgcctttccaatagtccctcaaagtcttaactcatttcagcattaacctaaaagtccacagtccaaagtctcatctgagacaaggccttccgcctatgagcctgtacaatcaaaagcaagctagttagttcctagatacaatgggggtacaggtattgggtaaataaagccattccaaatgggagaaattggccaaaacaaaggggttacagggcccatgcaagtctgaaatccagtgagggagtcaaattttaaagctccaaaatgatctcctttgactccaggtcttacatccaggtcacgctaatgcaaaaggtaggtttccatggtcttgggcagctccacccctgtggctttgcagggtacagcctccctccaggctgctttcatgggctggtgttgagtgtctgcagcttttccaggcacccagtgcaagctgtcagtggatctaccattctggggtttggaggacaaaggccctcttctcacagctgcactaggcagtgccccgatagggactctgtgtgggggctctgatcccacatttcccttctgcactgccctaagaggttctccttgagggccccacagcttccaccctctgaaccatagcccaagctatgcattggcccctttcagccatggctggagcagctgggacagagggcaccaagtcactaggctgcacacaacatggggaccctgggcctgccccacaaaacccctttttcctcctgggcctccaagcctgtgatgggagaggctgctgtgaaggtctctgacatggccttggagacatttccccatggtcttggggattcacattaggcttcttgctacttatgcaaatttctgcaaccagcttgaatttctccccagaaaatgggtttttcttttctgtcacatagtccggctgcaaattttccaaacttttatgctctgcttcccttataaaactgaacgcctttaatagcacccaaatcacctcttgaatgttttgctgcttagaaattttttccaccagataccctaaataatctctcaagttcaaagttccacaagtctctagggcaggggcaaaatgtggccagtctctttgctaaaacataacaagaggcacctttgctccagttcccaaaaagttcctcatctccatctgagaccacctcagcctggatcttattgtccatatcactatcagcattttgggcaaaaccattcaacaagtctctaggaagttccaaactttcccacattttcctgtcttcttctgagcccttcaaactgttccaatctctgcctgttacccagttccaaagttgttccacattttcaggtatcttcagcaacgtttcactctactggtagcaatttactgtattagtccattttcacactgctgataaagacatatctgagactgggaagaaaaataggtttaattggacttacagttccacatggctggggaggcctcagaatcatggtgagaggtgaaaggcacttcttacgtggtagtgacaagagaaaatgaggaagaagcaaaagcggaaacccctgataaatccatcagatctcatgagacttattcactatcacgagaatagcatgggaaagaccggcccccatgattcaattacctccccctgggtccctcccacaacacatgggaattctgggagatacaattcaagttgagatttgggtggggacacagccaaaccatatcaagacttctacatatgaattttggagggacacaatttaactcataatagtggactgtcytgttaaatgttctgtgtgcacttgagaagaatgtgtgtattctctcattgttggattcagtgacctataaatgttaattaggttaaactaattgatgtagggaaaagaaagagagatcagactgtcactgtgtctatgtagaaagggaagacataagagactccattttgaaaaagacctgtacttcaaacaattgctttgctgagatgttaatttgtagctttgccccagccactttgccccagccactttgacccaacttggagctcacaaaaacatgtgttgtataaaatcaaggtttaagggatctagggctgtgcaggacgtgccttgttaacaaaatgtttacaagcagtatacttggtcaaagtcatcgccattctctagtctcaataaaccaggggcacaatgcactgcggaaagctgcagggagccctgcccttggaagcggggtattgtccaaggtttctccccatgtgacagtctgaaatatggcctcgtaggatgagaaagacctgactgtcccccagcccaacacccataaagggtctgtgctgaggtggattggtaaaagaggaaagcctcttacagttgagatagaggaaggccactgtctcctgcctgcccctgggaactgaatgtcttggtgtaaaacccgattgtacatttgttcaactctgaaataggagaaaagctgccctgtggtgggaggtgagacatgtttgcagtaatgctgccttgttattctttactccactgagatgtttgggtggagagaaacataaatctggcctatgtgcacatccaggcatagtaccttcccttgaacttaattatgatacagattcttttgctcacatgttttttgttgaccttctccttattatcaccctgctgtcctactacattcctttttgctgaaataatgaaaataataatcaataaaaactgagggaactcagaggctggtgccggtacaggtccttggtgtgctgagtgccggtcccctggactcactgttgtttctttatactttgtctctgtgtcttatttcttttctccggctctcatcccacccgactagaaatacccacaggtgtggaggggcaggccaccccttcaattgatagtatggttcaagaacaaatggtatcaacttaggatggtttaacttatgatttttcaactttagaatggtgtgaagtctgtatgcattcagtagaaggcatactttgaatttttatcttttcccaagctactgctatgggacaagatactctctcacaattctgggcagtggcagcaagcctcagcttccagtcagcacccaatcccaagggtaaacaactgatacagccattctgtttttcatttttagcaaaatactcaataaattacatgaggcactcaatgctttattataagacaagctttgtattagatgatttgcccaactgtaggctaatgtaggtgttctgagcacatttaaggtagactatactatgccatgatgtttggaaggttaggtaaatttaatgcattttcgacttagaatactttcagcctcccaaatagctgggaccacaggtgtgtggcaccatgtgtggctaattttttgtggagacaaggtctcactrtgtcgcccaagttggtctcaaaatcctgacttcacgtgatccttccactctggtctcccaaagtgcgattacaggtgtgagtcaccacacctggccgtgttgtcacacattttaattctttataaattagaaacttcacaacacattgttcttatttaaaatttaaacaattatctttaaatatacataatatataaatatgtatataatatataaatatatacaatatataaatatataagatatattatatatataatatataaatatataatatataaatatataatatgtaaatatataatatataaatatatgtaaatatgtaatatgtaaatatgtaatatgtaaatatataatatgtaaatatataatatataaatatacataatatataaatatataatatataaatatataatatataaatatataatatataaatatataatatataaatatatattatatataaatatataatatacataaatatatataatatataaatatatataatatacataaatatataatatataaatatatataatatatataaatatatatggggaaaaaagcttttatacttactcatgtgattaccgtttcttgcagtctttattcctttgtttagatccttgggatttttgttgctatggtgaccttagttataccaggcacttcaaatcttaccttgtgtttaggawatgggcttgtttgcccaaaggtcttccctaatgtctgctccaccttcaactttaggtcttctctgctgtcagtttctctctccatacacttgtagctctcccaggagtattccatcgttacttgttagtcagtgcttattagcggggtggtgggatctgagaggtgaggtgcttggttgtgattctcagttctgattcctgcaggtactgtgtccctgggycttgaagggtatgaccaagcstctctgtccctccccgcagcagtagttttgggctcagcacatattcctgccccttccccagaatcagagggttttttgttgttgttgttttgtttttcaagtttttgttcctttttctccatctgtgttggatttaccagccccctaggagcgtcagtatttgttacccttcctccaggcttttaaggcctttgtaggagagatgggccaatagcatctgagcatggttttgtgtctttcttgtagcaactgatattcctcacccccaggtctatgccaggaaggatgcttccttacatgcctgtaatcccagctaccggggaggctgaagcagaagaatcacttgaacccaggaggcagaggttgcagtgagccaagatcatgccactgcactccagcctggcaacagagcgagacttcattcaaaaaaaaaaaaagctcttacactcttctgcatattctgcaaagtatactctgggaaggctggtttagaggatctctaacttttctaatatttcataatgcatggctgatgtttaacatgaactcagactcccatcagattttacagatctgggtctgcttttagttctaatgcttcagccagggcactttaatctgaggtcacagcattcccaattgtcagcagtattttgaaatctaagttcctctggtggacaacaggcaacatcatatagccayggaggatctgaacaacttctaagtctgagsgattttatctggtggttttggattcttttccatttatacttaaacaaggtgacacaaatgtcactcacagaaaagccactcttttttcccctgtttttaagaagtctaaaaatgcacaactatttcctgaatgagcagtgtgtccaggtgtagaatagggaatgtcgtgtccactgggaacagacggtgtacctggcacaattttatgagaattatcccatgattcctgtcccaccctgcagcccaccatgggctcagtggggagtctgagtctctgagccccaaagggtagctttttcccagacgacaccagcaagcagagcagcagcttggggctttgacaaggaggctggagtgaaaggtcatattggaccatcctggrtcactggaggacaataccccaggaacctccagcaatgactggtattggaatgactcccctggtcacccacctgtggcaagacatttgcaaacagggtcttcctatgaggcttgtgaggtttggccaatttgcattctgctagaattattaagtatggacatcctcatagatccagaaaagggccgagatccaaaggattgagaatgctttggggggtgtttccatagtgagtggctggctattcaacagtcaaatgtttgagaggatacaagtgatgtgtttcctgaggcaagtggtcagaacccaacagactcttctgttcagctaagatgagagaccatctgaagttcttacatgttctcacagagaaatggattctcatagggaaatggatatattgtgatagacactgtaagcagagaagttgactgagaaacacacacacacacacacacacacacacacacgtcaagactgaatgcatagatgtgtattataaaaagygtaaaaatacaccaccattstaacagattgtgtaggactatattttctttttttttaatttattattattatactttaagttttagggtacatgtgcacaatgtgcaggttagttacatatgtatacatgtgccatgctggtgtgctgcacccattaactcgtcatttagcattaggtatatctcctaatgctatccctccccccttcccccaccccacaacagtccccagaatgggatgttccccttcctgtgtccatgtgttctcattgttcaattcccacctatgagtgagaatatgtggtgttttgttttttgtccttgcaatagtttactgagaatgatgatttccaatttcatccatgtccctacaaaggacatgaactcaccattttttatggctgcatagtattccatggtgtatatgtgccacattttcttaatccagtctatcattgttggacatttgggttggttccaagtctttgctattgtgaatagtgccacaataaacatacgtgtgcatgtgtctttatagcagcatgatttatagtcctttgggtatatacccagtaatgggatggctgggtcaaatggtatttctagttctagatccctgaggaatcgccacactgacttccacaatggttgaactagtttacagtcccaccaacagtgtaaaagtgttcctatttctccacatcctctccagcacctgttgtttcctgactttttaatgattgccattctaactggtgtgagatggtatctcactgtggttttgatttgcatttctctgatggccagtgatgatgagcattttttcatgtgttttttggctgcatcagtgctctacacgttcagagaaacttctctagtgacgaactatagaaatgatccctgaaagtatagtcttaggactatattttcttttgacttgggaggcatgtttattgctgttaatgctgcaaagggctctacgtgctttaaaaaatcccaatctgttgcattcataagcctgggttggatctaaagcagcctcccacttttggaaaggcatccccacgacctttccatggttgctgaatgcagctggaggcagtcacagctggtgatgtccggagcccattccccactgtgctggtctgcagaacttctgcatgccattcccacaagcaggtctctgccctgctctcctccacctcccttgtcagaggaagtctgcacttcacagctttctggtctcaaccctcctccatccctacagatgtgtaagcagcaggaatcaaaaggtgaaggagagggggcaactcacctccgatggacacgtgaaaagtgggagatggataaaatcaagaaggagcttaagatatccagaaatgtaaactgtgtttggaaaagtaaggtcaggagaagcatgggactcctgaggttgctccctactatcttgcagacttgctgcaggaccaaatgaagcaggatctgtcaagcaccagggccagctcttaagcttagtgcctttctgaaccctgtgacccagcagcctccatcaactcgtcctacctgccatgcacagctcctctgtgcccctgtacctgagctcatgctattccctctgccaggatgcccttctccttctccaccaggagaagaacacttgccagtaagacccagttctaatgtcaccccttcctgacggtatcaggaagagtcastgatggtgttttatgctcccagagaatttgccacattgtgttgtgattatttttccacatctgtctcccccactggaatgagagcctcactcatcttcatacctccctggtctctacctggtgccagaaccatcctcagggcaggggaatgctcaggaaatagatattgaataaaataagtgtatccatccatccatccatccatccttccatccatccagacatatatacatatgtaaacattctgatggtcaaatggaacaatgtgggctgaagaataatgcaggtagaagaacctaagattacagattcttgatttgggaggaactttattttattgtgcaaacagtctacaaatttgaaacatactctatcaagaaagacactgttgttaggaaagggcgtgggagagggtggccccacaaaacagtgtaaagttctaaagaatttgagaggaacactctgcgggaccctgttccaagggcatctttctaagagtctgtcccttaggctctgcccctttttggccacgttgtcaaaggccttctttatgaacgaagtgagacacattatttttgttttcttgaattctaaagtgattgtcttgagttccctgggggagactcctgggaggtgtggcccactctattccagaaacaaaccaggagactgaagactcatcccaatcccatatcccatgaaaatgtgaaatcaaatcacccctgacaattccaaaactaayggaatcctaacataactctatgtaactctaaagctgagtgtgctggtaattaagcttccagcccacccttcccagctctggggtgcagggtcaccatgggctccttcctgtgtgcaccccaccccacccccactgtcagcatcctgtctccaccctgagagtgacagctggttccagtagcgggagttggttccagcttgccattttcctagcactcctataaccagcctgctgatgcccattcagagacagcagcacgggctggccatgtcccctctccagaattctgcgtccagctcctggaccttgagctctgagcccttgggccacgtgtacmattaatagtgcctcctcctcagaggactaacccccagccctagggccaccttcatatttcygagttttgatattttcaacctcttttctttgttgtatgagtccttgggctgggagcttgcagtcaaaatcttcatgatatctcattatcactacttttttttaaatctctactagctggataacaattatttatattaaattctctcttgaaataactgatacagtgtctcttgattgaaacttgactagtagactaagaattctaactctaaataattctgagggccgggtgtggtggctcacacctgtaatcccagcactttgggaggccaacgcgaatggatcacctgaggtcaggagttggagaccagcctggcaaacatggcgaaacccggtcaggagatccagaccatccyggctaacatggtgaaacctcatctttacttaaaaaaaaaaaaaaatacaaaacaaaattagccaggcgtggtggcagatgcctgtagtcccagctattcaggaggctgagacaggagaatagcgtgaacccgggaggtggagcttgcagtgagccgagattgcaccactgcactccagcctgggtgacagagagagactccgtctcaacaacaacaacaacaaattagccgggcatggtggcaggtgcctgtaatctcaactacttgggaggctgaggtaggagaattgcttgaacccgggaggcggaggttgcagtgagccaagattgcggcacttcactccagcctgggtgacaagagcaaaactccatctcaaaaaaaaaaaaaaagaaaaaaaaatctgacaattaaataaagaacagaaaaaaaatttgaatggcaaatacaaagctgaaaagaaataactgagaataaataactctgaaaatagctcaaaaactaaatacctcaaaaactctttaaaaattcagaaaatataatgttcaaatatgaagtaatgctgaaaatgaaataactaaaaaccaagtaacttgaaaaaaagaacataaaaataaacaactcaaatataaaataactgaaaataaatacctgtgaacataagcaactcgaaaaccagataactaggggaaacccttcattaaaacatttcactctgaaaataaataacttgacagtagttcatgaacttccagtgagtgtttaatagtcaaataagttactgtaaaaataaataactcaaaaactccaataagataaagtgaaataactatgaaggtaaataactcagataataattgtaaagataattaaaaataaattccggctgggcgcggtggctcacgcctgtaatcccagcactttgggaggccgaggcagggggatcacgaggtcaggagatcgagaccatcctggctaacacggggaaaccccgtctctactaaaaatccaaataaaaaattagccgggcgtggtggcgggcgcctgtagtcccagctactcaggaggctgaggcaggagaatggcgtgaacccgggaggcggtttgcagtgagccgagatcacaccactgcactccagcctgggcgacagagcgagactccgtctcgaaaaataaataaataaataaataaataaattccaagcaaattaatttgaacatctgtaactcctaaaacaaaacaactacatataaataactgaaaatgaacaactaaataactttgaaaataaagtaactataaaccagtaactgaaactataaaattaaataaccagaaagctacaaagaaatgaaaatcaactaataaaactaaaaataaaatacaactgaaactaacaaaactcatttaaaaactaataaatcgatacataaaatacctcagaaagtaacttggaacgaactctctctacaacgaagcaatcttggcactaacacgaacaccccatgaacgctcgcagggatgctgggaggcggaccgggagctcccagtctgcggcccccgccgggttkgagcggctccggctcctccaaggctcgggctaagcggctctcaaccgattcccaccccgccctggagaaatgcgggcgtgtctgcaggcatttttgattgtcacgatttgggggcggggtggaggatggggtggcgcattcctggcacctagtgggtagaggccaggggtgctactaaacatcctaccgtgggagcacagagaagcccacgcagcaaagaattgcccggctccgaatrtcgaagtgcgcggtcgagaaggcgtgggctgcgggctctgctcgcctctgcaggcgccttagagcagctccgaggtcccccgtgcggagctaggcgcgcacccaggacacccctcgggctcctcggaggaggccctggttgtcccctttctgccgccgccgaggctccggctgctttctgcgtagctgggcagggcccgggcccccacaccgcctctcccgggaatgcgggcgctctggagccgaggagcgggggcgtccgcagggaggtcagctctcctgggcggaggtcctcgggcgcagcgccctcgcctggaaaccagccgtcgcccccgcaggagccagccggcccgtggacgccccagcgcgctcctcctcggtgctgcgggtcgccctgcaattccgagaagaaagtcagagacgccgtggcccaaagaggcgcttagtctttcctcgctcacactcacgtttcctcctcatcgcgttcttctttttctccctggctgctttctcccctctccaggaaagcagatttggaggaacaggtttcgtgactgtcgtccgactggaaaaggcccgcgagctggaagggaggggacgggtgcaccctcagagttattgctggaggctgtggccagaccgggcaaggtggtgactcccgctggcaggctgaggcccaccccagccctcccacctgggccacggggctctcagcgggagccccagttatgaccggacaccagcgcaccgccaaggagacagccacgtggggacatgctggactaggagggtcagagccagtttgagggtctggtgacctcggctccctggcttaaccaggtccttatgggtgagaatcctgaggagggggagagggatggaggctagggacaggaggcaggaggagctgatgaataggaaggagggaagaagatgaaagaaacaaaagggaagtaattacactcagagcactgctctccttttccatgtcttctgcgccttcacagtcttcaaaggtgtccatggagctaaactccctgctggagcagccttagggagagaaagggaaggatggggcctctgtggggtgggaggacatcccctctgcccatggcagggtgtagcaggcagtgcctgttgcaggcacggtcctccccatctctaactcctgctctccaagggcctgcactgcgctgggctgtgagggggtctgtgatctccaggctgcttttccagcgccgagatgccgtaattcaccgagaaggcgcgtccacatgctgttcatggccctacctccccgttcctccaagaaaacagtcattgttttttgtgtttgccagtcttctaaccacgcctccttcccttcctcctcccctgtctctttctcaccctcccctccttgcctccttttctctcccctaattaatgtccatttcccatctccctggcagcctctgccaagtgtcactgctccccataagggaaaatcagaggaacaagcaagtgcatccatcctgcctctctctgcagtgaactgattaattaatccatcagtcttgtctatggcgcacatgttacatccctggggcggtgttggacactgtggggaacagcagccactgccaaatactgaacaactgccctgtgcctggtggtatgttaggcatttgtcaaagtttaagcctcacaaccctgtaagggtctcagccccctttacagttggggaaacagacagcaatggtcacttggccaagtcctcttggcctgtggcagggcagctgtcttctccagcactcctgctctttaccctcgctctgagtgagatggagtctctgccccacatggctcacaagccagggtagggagcagagcatctgcagaaaggtcccaacacaggacagccccagaccgagggtcagctgagtagtctacgcggcgggagccgtgctaggaaagtgtctgctcaggcgagaattcagggaggtttgggcaggacttgaggggcagacaggactggagagggagggcattctgggcagaggcatggccagagggcggtaggcggcagtggagggagctgcagttatctgggtgagcaggcagcacaagtrgcgtctcctgggctgctgccccaagcccccaacaagccacgttctgggccccaggccctccccagagcagatcagtgggggctgtgtgagtaacatgggggcgggggggcagctgggcagcacctccctggaggcccctctgaaatcctgcctgactctggcaggctccgagggggctggacaccctcctctcaggttgaagcaagtcctggttgagttcctagtcccaggaggtgggaggggcaaggggtggagggcagaggagaaactgcctcagggatgtgccccctgccttcatcctccagacaggacttgggagcatctaaggaaacccaagactcctctttagagaagtcatccagccctggggtccccttatgccaggagcaagcagtgagaatggaagaatgattgtcttgctgaaagttctgtgatggagggatagagggacagagggagccatgcccttgaccatcccctgcatgaataggaagggctgtgtctccagggtccatggcctctgtgccccggatgatgccagggctgctagggaccatagagccacccactgggaggctggcggttgggcctggctcaggagccttcgtcagccataggcagccacagcctggggtgggcagggctgggaggcgacacaggaactgaaaaacctgacaagctctagcccctccgcagggtaagtggtacctccaggtaaaatgattagttkgttccagcccctctgcagggtaagtggcacctggggtaaaatgactgcctggagctggcagctgctttccctgctctcgcgggccctgcagggaagcggggaagggaagggggcacagcgctgggcacagaggggctctcagaccctggactcaactgtttcagggtcatctgaaacagtcaactgtttcctctagcccattccctgcctccaggcgaggatttgcctgaacgtggaaagaggaaggatcctcccagtgctgtcaaccccagattccacctccctgtgggggactgtcagcgcaggccctgacaacgcagagaaagacacaggacccacctgggccagtgacagcaggagctccgggtgccacaggtgagggtggggatgcctggagcaccacggggggcctggtttagtctagagccaggttttccatacaccttagagtgcaacctcagggagatgcaaattttaccccctaacacagcatacacgcagaaacacatttatacaattcaaacacaagcggacggaacaatatttacccttagagtgtgtgaagtcctcatctgtcccacctcatcctatcatgctttgttctatcctaggagacaaagcaggagggggggctgcggaggtgggggagtctcatccaagcccttgggtgacacgtctctcctgagacaaactgcagctgctctgggtgtgccctcgcctgtctccctccaggccccgggttcctgccagcagagacagtaacctattcaccaggtatcccccagggctcctggaagaaactcagaattctcagaaccagaaaaccttagagagcatcctgcaggcaaagcccctggttctctgcggaggaaagtgcggctcacagggtgccccgccagggatggtaattgacyaccaggctgtgtgccttgtggggactggcttaaggccctgtgggagctgagtcagggccaggaccggggtgtcctgactcctagagatcatgttcccttcctcacccaggccttccagtcccagccctgggcttttatttatttattttggagacagagtctggcttgtcacccaggttggagtgcagttgtgtgatcacggctcactgcagccttgacttcctgggttgaactgatcctcccacatcagcctcctgagtagctgggaccacaggcacatgccaccacacccagctaatttttgtattttttggtagagatggggttttaccatgttggccaggctggtcttgaactcctgagctcaactgatctgcccacctcagcctcccacagtgctgggattacagatgtgagccatcatgcccacctcctgggctgacttttgctgtcttacatcatctgcatatttaatcccctgctggattcactggtcatgggctctgaggccctaagagtcttaggcactaaggagctggcagcactgaggggaccccaaaatctcagactcaggatctggccagtcacaggcatgtgagggaacaactgagaggcccattgccccatggcaggagaaggtgctctggagtcagtcagacctgagggcagtcagacctgattctcactctgtcactcactagctgtgtgatcttggatacatcacttaacctcttgagcatcagcttccttatctctaaaatggagataataacatcgattttgcagtcttggtatgaggattagcaaatcttctgataaagaaaaatgcctggtacatcataggaattcaacaaatagtacctgttatgattattgtgtatagcaattacaataatactaaagagagggtctcaaaacagctctgggcactccaggtgtgctattattacttacatttcagggaggtttgtctgccattgtctcatcctcataaacactcagggaaagaaacattataaggataataaatggctttaaaaagaaacagagcaaacacacacacacacacacccctcagaaaaaccatgccaaacacacaggctcttgacaaatattcaatctgattatagcaaaactgttttgttttgttttgtttttttgtttgtttttgagacaggggtctcgctctgtcgcccaggctggagtgcagtggcgtgatcttggctcacagcaacctccttctcccgggttcaagcaattctcctgtctcagcctccagagtagctgggactacaggcacatgccactatgcctggctaatttttgtatttttagtagagacagggtttcaccatattggtcaggctggtctcgaactcttgatctcaggtgatccacctgccttggcctcccaaagtgtgagattacaggcgtgagccaccatgcccagctgattatagcaaaattctaagtgatagttgtattcttggaaaatgaatggaacgacttttgtcccagccaagatctagtggtgtgttggagcagatacaccctgagtctctggggcactctcagtctatgtatcagataagcataaggagtattggtggagaaggacaagaatgggaaaggtgggcgatgagaattcctgcagataaggactggtgagagtattctcttttgaaacccttagtcgacaatctttctggtgcatatcagataagctgaatggtttaggaaatctagtgttcacatttagtgcttagaattctaagcttttttattttgcttaaacaaatggaatgaaatttattaacaagtgaacctagtaatgagctgaaattattctcaccagcatacatatttttggtaaattatagactttgaagacaaaatcatggtgtttcccttactgtccagtggatggcacaaagagaccattgtagatcctgctggttcagcggtagctctcaatccatagatattaaatgggcaaattctatttttattgtctttcaaactagatttttctcaatgcacaactttttttttctttttcttttttttttttgagacggagtctcgctctattgccaggctggagtgcagtgacacgatctcggctcactgcaacctccgcctcccgggttcaagtgattctcctgcctcagcctcctgagtagctggggactacaggcgcatgccaccatgcccagctaatttttttgtaatatcagtagagacagggtttcaccatgttggccaggatggtctcgatctcctgaccacgtgatccgcccaccttggccttccaaagtgctgggattacaggcgtaagccaccgcgcttggccaattatgcacaactttttaaggaccatctctatcacatgaactaaaggatatcattttcacttgggggtgggaagtggtgagctgcttaaaagcaatgcctaaaccccctgggctttccttcctttcacttggaagaaccagggggtaactaactgctaacaattccatgctttcagagatctaggcgcaagcctaagggcttcatctcatttaatcctcatggcaagactgtacagtattatctccattttgtaaaatgaatgataaaaagaacttaagcacagacaggctatagaatccatccaaagagatggagctgcacttgaggctgggtctttgagacctgagtttgagcccttcatcattgtgttgtgtgtgctgctaaccaatttcccctctctgtcttctaggatttaatacatggtgtcacattctgtctgatccatcccagtagctctccagagctcccaacaggagagagttccaaaatgtttccaggggtactaggctcggttacaatattttgcttggtggcccagagtataaacgtggatatcttaggctggtctatagctgaaacgtctatttcatttgcaagcctatctttggctaagaggaagtgaatcattcttgagaacatctaattaattgctttcagattccacatgttgacattctcagggcacattttttttttcccgtgcatttctgttgtcaaagtccctgccagctcctaaggcagtctgagctggctgtcttagactttcagagctgctggaagcttgggggagggaggggctgtaggtcaaagaaactttataacctagctttacctcccagctcagccaccagctgccctcaaatgttctggattggaataagcccaaagatgagtggcaggagggaagggcaagccaatacgtctagtttggttcagtcaaagccttgcccatttcatcagaattttaatggaaaatttccaataagattaaaatataaggtcaccccaattttagtatggctccatttaaaaaaaatcatgcatatctttgttttgcaatggggccttactcttgcccaggagaggtgcggtggtacaatcatggcttacggcagcctcaacctcctgagctcaagcaatcctttcaccttggcctgccaaatagctaggattacagacacccaccaccatgcccagctaatttttaaaaaaaattttttgtaggtccagtgcagtggctcatgcctgtaaatctcagcactttgggaggccgaggcaagcggatcacctgaggtcaggagtttgagaccagcctggccacaatggtgaaactgccgtctctactaaaaacacaaaaattagcgaggtgtggtggtgggcacctgtaatcccagctactcgggagactgaagcaggagaatcaattgaacctgggaggtggaggttgcagtgagccaagttcgtgccattgcactccagcctgggcaacaagagtgaaactccgtctcaaaagtttttgtggagatggggtcttgcgatattgcccaggctggtctcaagctcctggatatcaagtgatcctcctgctttggcttcccaaagtgttgggattacaggcatgagccaccacaactgaccaaatcatgcatttctatagacaacgtctgcaagaagatattaatggtgattatatctggttatgttggatttgtatttttatttgtactttcctgtattttctaaattcctgacattctacatgtgtactcctttaataatcagaaaaggaaacttaaaatagttaaaaccaattggtcagatatgtaaaataacccaccatctctccagagagggctgtttgctagaacttattttcttcattgaaatactagagtgccccaataagtttgaataacacaaaaaaaaagataatgaaagtaactaaattatctaggcccaaaaggaaatgccacaaaaattggcaaagaaacaaccatgacgtgctataccggatagctcctaggcccccttggagaccctgaggtacccgacgagggacctgtagtaaggctggcagacaggttcttcctctgttagctctgaggtacaacagttattctcatttttatgtctttcacatggccagaactttgcaaatcagaggcaaagtgaattcagaattaaaaattttcagcaccatccaagtcagtaaaacagtcttagcttataacctttattttttttattattttattatttttttatttttggatggagtcttgctctgtttccaggctggagtgcagtggcatcatctcrgctcactgcaacctccgcttcctgagtccaagtgattctcctgcctcagcctcccgagtagctgggacgacaggcatgcgccaccacgcccagccaatttttgtatttttagtagagacggggtttcaccatgttggccaagatggtctcaatctcttgacctcctgatcctcccgcctcggcctcccaaagtgctgggattacaggcgtgagccaccgcgcccagccctttatttttactgtaagctgccagtaaacagcacacccacctgtctgctgactgtcccatctggaagttgtgtaggtccttactgaatcttaccttcttcccttcccctacccagaccccatcccagctggcacgtggaatcctttccctttctcctcatgtcaatagaccagcaggcaaagagagcagttaccatagtggaaggagaaacgggtcctagtcgtggtgaggaggtagggctgctgcttaacaraggccgagagggacatgtctgatgctcaggtgatccactaggacatctcgcaatactcccatgcccagctgtaactatgatggccaagtccaagaaccatagcctgataagagtctaggacccccactatacatgccaccagtagaagtgcctgatggggagagggatgtctagaatgagtaggagggaatgatgacaaagcacagccaaggaccaactgcagtggcaggtggcagccacagattgagttaagtaggattcttgctgtgttccctagtggaagcatctcccttgttgagacccactgaggttaaagttggagacatggggagaaaacaatgtccccgtgttcatcactagagtcatgatgggcaaggtcacaactcatttgacttcttggctcccagattcatatactctgcctcttgggggcacttcaccataatacagccattggttcagttgtatgctgcatcctgagggacagcactccatccccgcagtgccatgtccaaactgctgccccgcctcactctcaagaggctgtcccactactctgtcaggcaccagcttctgaatgcagtggtaagactctgtcctgtgatcacgtctatggctgattctcttttgccatactgagtcccctgatcaaagcagtgttatacagcctggcgcgctggctcatgcctgtgatcccagcactctgggaggcagagggggatggatcactagaggtcaggagttcaagaccagcctggtcaacatggtgaaaccctgtctcctctactaaaaattcaaaaattagctgggtgtggcatgagcctgtaatcccagctactcggaaggctgaggcaggagaatcacttgcacccaggaggtggaggttgcagtgatccctgatggtgccgttgcactccagcctgagcgacaagtgtgaaactctatcgcaaaaaaaaaaagcaacgcttatgattgatcacacactgtactttctaacctgaaggggtctggtaaaatcaacttgtcttctcaagggacagtaccaaactgggggctcagcactgacctctgctggcatgtcggacatttaaaagtggtagcagctagagcagtcttgaagacatggagccctcactgctggggccgagcactgctgggttcaattctgctaccaaggcttctccatttccgagcccatggtgcaggcactggggtgcctaagaggaagtgcactaactggacttctgctatgcaccttctttagggggcattaaagcacagttcaaagactctcacattttgagttaacccccacaggcctgtgtgccccttctccagtcaggtcatgttgtcatttgatcctagttattattctgaaagggttcaggaagggaggtggggacagatcttgraaagggcaaacattgccttgtatgcctcatctgaggcttccgcatagtcttaggggactgggaaccgtcttaagtgaatctcaccttcttcccttcccccagccaggccctagccctactggcatgaggaatcctatctctttgtcctcatgccaggagaccagcaggcaaagagagcagttaccatactggaaggagaaatgggtcctggtcatcagaaggaggtactcctcaagggacagtacctgagggagacacttccactagggaacacagcaagaatcctacttaactaaatctacggctgccgcctgctgctacagttggtccttggctgtactttgtagtacaaggcgaagtaggccactgctgccagcccaggaggtcaagggcatttacccagacatcctcattcccagtttttgggtcccttatcttccctctcaggatctaatgactcatctactggcactaggagtccaaaatgaccagatggcagactcgcctaggctgagaattcttccttctctcaaattctgccacttttaccagcccagtctgcctctgctgtaggagatgagaggctaaatgttggcagggaggccccctgattctgatcctgtgttttaaatcgataattagtcygaccctgttactttctcccttcagtgcatcaacgggaggctcttagcaacagcctcccaactctaccttcctgatagataatattttcctcaaagctctcaaatgctagagacattgcaccagctggygtgtccccatccacctgtatctgatcccaggtcaccacaggtgaaggtctgagcaatcgaactgctacagcaggccaggtaccaggactctcarcacgccacttaacaccaataatgaggtcctcatggccatttggcctgcagtgagccaacaccagaatcccatccattctgggacccctcctgctactaacggtcttcagtcaggttccccagaagcagattatgcaaatgtgttattgaaaaacggctttcagatgaaacctggaagacagtgaaggccgaagctagggcagggaagaagccaggcacagacgtgggcttagcagaagtctagcatcagcctgatcccctgggcagtactggagcatggatggcaccacaggttaccatcttgagacagaaggactggcttctgtaccctgaatcagtaagccattggtgggccataagccacccttaggggaggacataacctcacaggcatttcctggctggacgaccctgggcagctgagggcaactgcctgaagggcacaacggtgagccattgtcagctaacctcacagcagcagagacatggggccaacagcctataaagagggtctgggcaggctgtcaatactctctactgtaatactgtatatgtggtttattttaaaaacttttttagaaggctttattttattttattttttgagaaggagtttcgcccttgttgcccaggctggagtgcaatggcgcgatcttgactcaccacaatctctgcctcctgggttcaaggaattctcctgcctcagcctcccgagtagctgggattacaggcgtgtaccaccatgcccggctaattttttgtatttttagtttcatcatgttggcctggctagtcttcaactcctgacctcaggtgatccgcccacctcggcctcccaaagtgctgggattacaggcgtgagccaccgcacccggctgactttatttttttagagcagttttaggttcacagaaaaactgagaggaaggtacaaagatttcccatatatcccctgtgctcacacatgcatggcctccctatcaccatcccccaccagagaggcacatttgttacaatggatgaacgtatacactgaatatatcatattcacccaaagtctgtagctcacatcatggttcactttggctgttgtacattccttggatctagacacttttataatgacaggtacagtagtccccctttatcctcaggggctacttccaagatccccagtggatgcccgaaaccgcagagagtgccaaacttgactgccatcagtgggaatatgtttctattcgccttccaccaccaccggtttaacgcctttttcatcttagtgctgctgccgtaactttggcagtttgagatgcgacagcaaaatgagtacaaatttctttctccttcttcacaatgtcatggacagatgattccttcttaccatagatcttagcaacctcggtgtgtgattttttttctttcttgttaaatcaactttcaccttttcacttaaaggaagcatttgacggcttctctttggcatatctgaatttccagcatcacgactgtgctttggggccattgtttgtttatttatttatttattttatttatttttttgagacagagtctcgctctgttgcccaggctggagtgcagtggcgtgatctcggctcactgcaagctccgcctcccaggttgacgccattctcctgcctcagcctcccgagtagctgggactacaggcgcccaccaccaagcgcagctaatttttttttttttttgtatttttagtagagatggggtttcactgtgttagccagggtgatctcgatctcctgacctcgtgatcctcccgcctcagcctcccaaagtgccgggattacaggcatgagtattttatttatttatttattttttcagacagagtctcactctgtcggccaggctggagtgcagtggcaccatctcgctcactgcaacctccgcctcccaggttcaagcaattctctgcctcaaactccggagtagctagaattacaggtgcacaccaccacgcccggctratttttgtatttttagtagagacaaggtttcaccatcttggccaggctggtcttaaactcctgacctcaggtgatccacccacctcggcctcccaaagtgctgggattataggcgtgagccaccgtgcttggccttggggccattattaagtaaaataagagtcacttgaacacaaacactgtgatcctaacagtcgatttaatcaccaagatggctataagtgactaaggctggcggggtggggagcacagacagcagggacaccctggacaaggggataattcgtgtaccaagcaagacacagcggaaggcgccagatttcatcgcactactcagaatggcatatcatttaaaactcatcgattgtttatttctgtaatttttccatttgatatttggacagcagttgactaagagtaactaaaacctggaaagtgaaacagtggataagggggtgctcctgtacttctcaatgtgagacatttgcccttcatgttaattctgccccattagytctacacaaatgaatagcaggaaattgattttaaaccacatggtggtaaaatgctttcttttttctccctcatttaactaaagaagggctgggccctaggtgatgtcttccttagcatctaagcagctggcatcaccccactgcttctgtgctccactctcccatgggaccctccctacctttaatccctcctgtgctggaggccgggtcttccttgctagtggtagctctttccatatttttaatccatggtccggatcctgctccactgcctttgctttagagaaataaacatgaatattgagtcactggaaggaatgacacacgcatccctcccccaccagttggagtaacgctggcccacctagtgtatctctggtctaggtctcgaggacctgctgctcctccctcacctgtagttgaagacctgcctcaggtcagtaggtgatacgcagtaaggaaatgtccaaaggacacttcttgttggattacacagcaaacatctaattggctgcaaatctttttttctttttctttctttttttttttgagacagagtctcgctctgttgcccaggctggagtgcagtggcgcaatcttggctcactgcaagctctgccacctgggttgacgccattctcctgcctcagcctcccaagtagctgggactacatgcgtgtgccaccacacccagctaatttttgtatttttagtagagacgaggtttcaccatgttggccaggatggtcttgatctcttgaccttgtgatccgcccgccttggcctcccaaagtgctgggattacatggctacaaatcttaaagggggaagagatgaggaggaataatcccctttgtcttctcaaaaatgttttcactggctcactacagctccctccttcctctttactgacccaaaatgccaactattatagtaactcttttgggttagacgaatccagtgaataaacacctactaagcattggaggtccaagaggaataagatatgcctgagctaaacctcatcacccccggccttgcttgcagagtggtttgctggcctcatctgattattcaatgagtgctttcatttgtttattcactaaatatttactgagcacctacaaaagtgcctggccctggtagatgaggcctgagggaagctaaaactaataagacaaactccatgccctagaggaattcatggtctcatagggagaaaatgtaaacacatcataaaattatagcttattaaatgctgcaatagagtactccgtaagagtgtgggggcagagaggagggggagaaacagctgctttctagagccctcaccttttccacttctctcatctttctgggttagggtctagcgggggttccatgaggatcaggctaaatgagcttagaaaaactaagcagactactgtatcagaactggatcacagtagacaggcgttttcaacaaacatatattgagtatccctgagtgctttggacaggaagaggaattatagacgaagattgtaagtcgcagtaatagatgaggcaaggagcaccagtgaggacttaaccctgaaagaagtgtgagcacatcttcctcccagacaagggggaataaagaaaggaagatgatcaggagagttctaagtggaactcagcagccagaggggaagcyggaggaggtaacatcagagggtgcgtttgcccactcagtaaagtaggaggcagggcagccttgtgaaaataggaatggaatagaaagctcaagaaaacagccaaaagcaagggcaattaggggaggtttcgaactggcagatctacctcaactggcaatacagcaagcatgcaccagaaaagatatcctagctagcagtggggttgggaactgatttaatatcctaggctagcagtggggttgsgaactgatttattatttaattttttatatctattcatgtatttatgcacttatttatttttgagataggctctcgctctgtcacagaggctagagtgcagtgatgtgatctaggctcactgcagctttgacctcctgggctcaagtgatcctcccacctcagcctcctgagtagctgggactacaagcacgccaccacctaggctaatttttgtatttttttttgttagagacagggtttcaccatgttgcccaggctggtctcaaactcctgggctcaagcgatccatctgccttggcctcccaaagtgctgagattacaggcgtgagccactgcgcccagccaatattttttaaaaattggaaatcccttgtaataagccaagtgttgggggaagaaaagcaataaaagcaatgacatggactcaatatgaaacatccaaagcatttgacatgcctttaaaataaaaaaatcagtactcacctcgtgctcatttcaaactgtggatttccttggtctaaaattttaaaaaacaaaaaacagcactctcaaatttaagctaatttgaattgattttacatagtgatttttaaatacacatatatgcataaagagaatactataaaaatatataaaggtgtgtaggtcttgatcatattttcccaggaattcagaaaacgctgctacagtccctgggctcacgtggtcctggcatcctccccaacgtctctaccccatcctgctgagtttctggcatgcattggttctgggtctgtccaagtttctactgttagctccattaatactgaattaaggaataaacagtgctcaaatgctcattttttccatgtgagcccaaattatgtgacttgatgagggaaaaaatcatgagtccctgggagagcgataagaatcacattctttactaaagtgttgtcccaggtatgagaataacaccagatctcaacctccagaggccccccactgcctcccacagcataaaagccaaattcctcggcccgatatttgaggccatctcaatctttttcctttcaccctatacctgactctcccaggctaggctcagatcgtcactgaatgtgctcacttcgaggcacctctgtgattttcaaggttcctgggcccaccttttactaccgatgtgactgccacatgttgcccagttgctaggatgggaccgtggccttgatttctgccgggactgaggctttgccttgtttcccacccaccctgctgcctgcccctgcactcttctggctggggcctgatctttccccgcagtctcccttcacctctagatcacaggcagtcatgccacagctgaggagcttgtcccaaacctcagtgcctgcctccctcctcggcttcttgtgctgctgtgtctcacccatcagtgaygctcttctcttccctgcccaagccctagctgactccataacacctggatacaatgtcctcttctgtctggttacctccgagaggccctctctctacatccctcattggctcccagtgtcattcctctcacccatggccctaaggtcactgtattctttggccagtgtacagggttattatgcttaacaatccacaaaggttgaaaggtgttgtaggatggtgtaaaaatgaatctgggtggtaatgtttatatgtcagagctttgtaaagtgctcggcaggcgtaaggtactgacagtcctgatattcctgatcttggaacctgggacaccatcttctcaacattgcccggataccctcaagggtatccagacagcctgagtttgcattctgttcctggttcagcccagggccctggttcccgctcactcaccatccactgtgggccctctctaaattcttaaagcccttgccatttgcatcactcacagagacatttcatcagagcctacttggtgcaccaggctcaggagactcagttctgctgcggataagttatgtaaaattgaccctctgcttgcacatctgtaaaaggaaggggctggcacaacacctctgggcctttcagttcagtagtgttttctttttatctaaaccacgtgctgggtcctggttttgcttcttatctagatttttgcattcctgtcacaacctataaagcacagttcaggccttaaggagggcttgagaaatctctttccgaattgtcaactgaataagtgtcatatcttcataacaaacttgtctttttttgcagggccaggaaggcagcaggggagtcagttaaaatataaattttagattaagcttaatattgttaagaagtcaattctcaccaaattgttctagagattttacataatcctaatcaaaatctcaataaggttttttgagaagttggcaagcagattctaaaatatatatagaagtataaaggacacagaataatcaaaacaactttaaaaaggaagaacaaaattataggactctaactacctcattttaagacttattagaaagcagcagtaaccaagatagtgagggactgatgtcaaggtagacaaatagatcaatggaaaagaatcaggcatccagaaatagatgcacttacataaatcatcaattggagaaagaatagtgtttttaacaactggcaatggaaaaactagaacatcagtatgcaaaggactgaactttgatccatacctcacaccacatacaaatcaaaaacagaaacaaacaaacaaacaaacaaatactcaaaatggatcagagacctaaatgtaaaactataaaacttctggaagaaaacacagggagaaaatctttatggcttttagataacgatttcttaggcaggataccgaaaacatgatacatacagtttttaaaattaaatattataagaattaaagtcttttgctcttcaaaagtcactcttaagagaaaaagatgccacacactagaagaaaatatttacaaagcattaaaaggacatatatctaggatatacataaaaactctcaaaagtcaataataagaaaacaatgagcaaaagatttgaacagacacttcaccaaagaagagataaagatggcaaagaagcacataaagagatgctcaaccattagttactagggaaaagcaaattaaaacctaatgacataccactatgcccctattagaaatctgaaaatttaaaagactgacaataccaagtattggtgaggacatggcacaagtggaactctcatacattactatgggaatggaagatactataatcactttggaaactatttagaaatttcctaaaaaattaaacatacacctaccatatggcctaaccattacactcttaggtatttacccaagagaaatgaaaacacatgtccacacaaagacttgtacttgcatgttcatagcagcattattcaaaatagccccaaagcagaaacaaatcggatgttcattaacaagtaaatggataaagaaaacggggtctagccaaacaatgaaatactactcagcaacaaccaaaatatgtactattgtttacaaaatccaaatagatgaatctcagaataattatgcagaggagagaagccagaccaaaaaaaaaagtacatagtgtattatctcttatgaaattctagaaaatgcaaactaacctatagtgacagaaaggagattggtggtcacctggggtgggtgagggaggggcaggagaaagggaatgcaaagcagtgtgaacaaacctttggcggtgataggcatgttcattatccttactgtggtgatgccttacagatgtatacagatgtcaaaacttatcagattgtacactttaaatatgtgtggtttatcgtgtcattatacctcagtaaaggagttttaaaaattgtagtaaaaggtctctacctagaaaccttaataagctaaaatgtatgtccccaggactaaccctagctattctatagttctggggtggaacctagaaatctgcatttgtagcaagcccttcaaataatttgaatgcaggtggtccttaaaccaccctttgagaaacactgacctagtgagtaaggatttctaaacaagcttgtgactagaatgttgtttctgcagggaacaagtgactttttctactagagttgccatatcattctgtgttaagcctctaggacactcctactcaattaccatgacatatatttaacataatattaatagttgtaaaacaaaagtaaaccatagtttttttcccatcttatctgtatacaaagtaaaatcaatgacataatattgttactagttcctggcagttacctacagtttaaatcaagaacttatgttgcttggtttttgtgacagaaaactgatgtggtggctttccagttacactgcttggtttaccttttcccttatcttatgatgcacacccagctttgcacgttcctgagcagctgatgatcatagaagacctagaaaaagtgtgagccagttgcattcctgctatctctaaagaaaaaagttatcttttcatttttatgtgatttttcttacccaaaatgcaaaccactgccagtataaatgatggaagatagactttccacatgaaactggcatttcttcaccctagtaacattacctgaggggaaggtcagaactcaattgataaagctcgctgtggattttcccaccaaatatccctgcaaacaaagtgaaaggataaagcctgcttaaagatgatttgtaattgttgaaaggagtacagtgtgtgacatcagtgaaaatggtggagaaaaaacccgtccccaaattctcttctttgtgaaagcatggaaaaaactggccaaaaatggttagaaaatttattttcagaatatcgaaatttttttttccttttttttttttttattatactttaagttctagggtacgtgtgcacaacatgcaggtttgttacatatgtatacatgtgccatgttggtgtgctgcacccattaactcatcatttgcattatgtatatcccctaatgctatctatcccttccccctccccccaccccaccacaggccctggtgtgtgatgttccctaccctgtgaccaagtgttctcattgttcagttcccacctatgagtgagaacacacggtgtttggttttctgtccttgcgatagtttgctcagaatgatggtttccagcttcaccatgtccctacaaaggacatgaactcattgttttttatggctgcatagtattccatggtgtatatgtgccacattttcttaatccagtctatcattgatggacatttgggttggttccaagtctttgctattgtgaatagtgctgcaataaacatacgtgtgcatgtatctttatagcagcatgatttataatcctttgggtatatacccagtaatgggatggctgggtcaaacgatatttgtagttctagatctttgaggaatcgccacactgtcttccacaatggttgaactaatttacagtcccaccaacagtgtaaaagtgttcctatttctccacatcctctccagcacctgttgtttcctgactttttaaagattgcctttctaactggtgtgagatgttatctcattgtggttttgatttgcatttctctgatggccagtgatgatgagcattttttcatgtgtctgttggctgcataaatgtcttcttttgagaagtgaccgttcatatcttttgcccactttttgatggggttgatttttttcttgtaaatttgtttaagttctttgtagattctggatattagccctttgtcagatgggtagattgtaaacattttctcccattctgtaagttgcctgttcactctgatggtagtttcttttgctgcgcagaaactctttagtttaattagatcccacttgtcaattttggcttttgttgccattgcttttggtgttttagtcatgaagtccttgcccatgcctatggcctgaatggtattgcctaggttttcttctagggtttttatggttttaggtctaacatttaagtctttaatccatcttgaattcatttttgtataaggtgtaaggaagggatccagtttcagctttctacatatggctagccagttttcccagcactatttattaaatagggaatcctttccctatttcttgtttttgtcaggtttgtcaaagatcagatggttgtagatgtatgatattatttctgagggctctgttctgttccattggtctatatctctgtttttggtaccagtaccatgctgttttgattactgtagccttgtagtatagtttgaagtcaggtagcgtgatgcctccagctttgttcttttggcttaggattatcttgacaatgcaagctcttttttggttccatatgaactttaaagtagtttttttccaattctgtgaagaaagtcattggtagcttgatggggatggcattgaatctataaattaccttgggcagtatggccattttcacgatattgattcttcctatccatgagcatggaatgttcttccattggtttgtgtcctcttttattttgttgagcagtggtttgtagttctccttgaagaggtccttcacatcccttgtaagttggattcctaggtattttattctctttgaagcaattgtgaatgggagttcactcatgatttggctctctgtttgtctgttattggtgtataggaatgcttgtaatttttgcacattgattttgtatactgagactttgctgaagttgcttatcagcttaaggagattttgggctgagacgatggggttttctaaatatacaatcatgtcatctgcaatttgacaatttgactttctcttttcctaattcaataccctttatttctttctcctgcctgattgccctggccagaacttccaacactatgttgagtaggagtggtgagagagggcatccctgtcttctgccaggtttcaaagggaatacttccagtttttgcccattcagtatgatattggctgtgggtttgtcataaatagttctcattattttgagatacgtcccatcaatacctagtttattgagagtttttagcatgaagggctgttgaattttgttgaagacctttactgcatctattgagataatcatgtggtttttgtcttcggagaacactggaaattaaatgatggcttgcagcaatctggagagcatttattcaaggaaaatggctgtgtctcagtatgactaatgagctttttaacttgccctatttctatcctccccttccttggtggtagccttagaaatgaacagcctgcaatgatagtgaaaatcagcagtctggcagccatgggaggggcagaacaggaatgggggaactatggagcctcattcttagagaattatcattatttgatctgtccacgggtttctaggaatacctgacctgcaagtctgtctttattaggcctgactcagaacttgcccaatgtgaaaagtcttttccccaaaggcctttgtagaaaatgattacaggcaattgtttaacttcttggttgctcgaggtattggctaacagtggggcaaacatgggctaatcagaaggtttaaaatgaaatgctcaggaataagatgctcatagagggttgtaaaggctccaaaatatttatgagactctagaagaccatgcacacatttcctgtgaacatgttcaggacagatctgcgaaggccccacgatcttacctctggctgatcttgatgatctgcacaaacagaaagtgaaagctaggctagaactgtcaagtgccaggctgagtgtgaaggtgtgccctaatgtgcacacagagccccttggcaaagactagaagacttactgctttcaggtgtttaaagaaatctctgtcatgtcattagtatttagatcactaagctaactgaacagaggcttcaatggctgcacataaatacagacttcacagaattagtttagaaaagtcactataacaaacaacaataaacagcaacaccaacaaacagtagaggtgggaaggtccaatttccagagttgctacattatgttatttaaaatgtgcaattttaacagaaaataatgagacatgtaaagaaataagaaaatgcagtccatacccagggaaaaagaaaaaccagtcaatacaaactgttcctaggccaggtgcagtggctcatgcctgtaatcccagcactttgggaggccaaggcaggcagatcaccagaggtcaggagtttgagaccagcctgaccaacatgttgaaaccccatctctactaaaagaaaaatacaaatttagccaggcatggtggcgtgcacctgtaatcccagctactcgggaggctgaggcagaagaattgcttgaacccaggaggcggaggttgcagtgagctgagatcatgccattgcactccagcctgggctacagagcaagactctgtctcaaaaaagaaaaaaaaagaaaaaagaaaaaactgttcctgaggaagccaagatactgactttactagaccaatactttaactatttttacatgttcaaaaagttaaaggaaatcatatataaagactaaaggaaagcacaagaacaaatcctcattaaatagaaaatataaagatttgttttaaaggacgaaacagaaattctagatttcaaaagtataataattgaatgagaaattcactagagtggctggctcaatagcagatctgagcaggcagaagaaagaattagagaactcagaaataggtcaattgaatctatccagtctgaggaagagaaagaaagaggaatgaaggaaaaatgaatagagcctcagagactgtgagataccttcaagcacgctaatgacgcataatggcagtctcagaaggagagagggataaatgggctaaaataatatttaaagaagcaatggctgaacatttcccaaatctgatgaaaacattaatctataccttcaagaaagtctataaactccaagaaatataaattcaaagagatcaagatccacacctagagataacataatcaaactgtcaacaaagacaatgaaattatcttgaaagcagcaagcacatagaaggactcttcaataagattaacagctgatttctatcagaaatcacagagttgagaaggcaatgggatgacatattcaaagtgcttaaagaaaaagagggtcaacaaggaattctatacctaaagccaatttatcttcaacaaagatgccaagaccattcaaagyggggaaagaatagtcttttcaatcaatggttctaggacaatggatatccacatgtaaaaaaaggaacttgggcccctaattcacatcatatacaaaaattaactcggaatgagtcaaaggcttaactgtaagagttaaaactataaactctttgcaaagaacactatcaagagagtaaaaagacaatgcacagtatgggaaaaaatacttgcaaataatatatctgataaaagtccagtatccagaatatataaataactcttacaattcaaccataaaatgacaagccaattaaaaaacatacaaatgagttaactgacatttctacaaagaagatataccaatggccaatatgcatgttaaaagatgctcaacatcactagccatgagggaaatgtaaatcaaaatcacaatgagatactagtacatgcccactaggatggcaatgataataataataataataataatgttattataatgaacaatacaagtgttagtaaggaaatggagaaaattgaacccaactattatattgctggtgtgaatgtgaaatggtggtactgttttgaaagatgatttggaagttcctcagaaagttaacaaaagttaccatatggtctggcaattctacatatatacaccaaagaaaactggaaataaagattcatacaaaaatctgtacaaaaatatctacagcagctttacttacagtatccaaaaaggggaaaaamcccaaatcttcatcaactaatgaatggataaacaaagatggtatatccatacaatggactgttactcagccataaaaaggtatgaagtactgatagatactacaacataaatgaaacttaaaaaaatacgcaaagtgaaagaagctagacagaaagggccatatatgtgatgtgtagagaaaacatccaatagagacaggaaatacattagtgattgccaggggctgggtaaaggtgagagatggagaatgattgctaattggtacagaatttttttgttgggagagttatgaaaatattttggaagtataattagaattaggctgttaaatactttaaaacaaaaaacagctgggcgcagtggctcacacctgtaatcccagcactttgggaggccgaggcaggcagatcataaggtcaggagatcaagaccatcctgcccaacatggtgacaccctgtctctactacaaacacacaaattagccaggcgtgttggcgcgcctgtagtcccagctattcgggaggctgaggcaggagaatcacttgaacctgggaggcagaggttgcagtgagcccagatggtgccactgcactccagcctgggcgacaagagcaaaaactccatctcaaaacaaaacaaaacaaaaaacaaaagtaagaagtaaaaaaatagaattagactggggatggttattgagccttgtgaataatctaaaacccactgaattgtatattttagacagtgaatttgatggcatgttaattatatcttgatgttttaaaaaaagtagtacagtgtctgaaaggtagcattggccatttgaatctctgttaacattttcttgggtggccatcagatttttgattaaaggtgaccttggcaattaagtggtggggggaacacaaccaaaagggacaaaccaacacaaccacacacaatcacacaacccctccacctccaccacagtggaaatcaagcataggtgcttccagggactgtgaaaatgtggccacagttgttttcccagcaaaccattttctttcacaatatccccaatttgcacatcccccactgagctgaaatcagtataactcgacagacraaagtcagcaggggtaacctacctttgatattattttgttgttccatcaataatttacttatttctgaaaaccaacagtctctgatttcttttgaagctgcctgcaatcacacataagaaaacactataagactaccatgagtattaattgcagagtttttattctcccagtgaccgttagactaatgagatagaaaacacttgtcaagtcatgggaacacaataaggaaagagtagaggagacccatggggaaggcatgagcgttacctgcaggatgtatttctcaagtccatttcgactggcaatctcaaactttctatggctcccccttccaagctggcgaattgaaagtgtcatcagctattataaagagggaagagaaatgttctataccatcgttttctgattttaaaatagtccctgtcaatcactctaatcctttctatccttgacagctctcttctttcatcttcctttcaagtttttgctttcctctcttaatgactccaagacagcataaaacacaatctggtacagtgtcttctgtctcatctgtttcagaaggtaacacagactgttaactttctactgtatctttatgcaggaggcgctgcagccctcaacctttcagtgaagagatcatttctccttcaacrtgtattttacaactttctacgtaatagtaactgaataaggatctattcagaaacataaataacaaggccaggtatggtggctcatgcctctgtaatcagtcctgtaattccagcactttgggaggcctaggtgggcggatcacctgaggtcaggagttcgagaccagcctgaccaacatggtgaaaccctgtctctactaaaaatacaaaattagctgggcatgctggtgcgtgcctgtaatcccagctacctgggaggctgaggcagaattgcttgaacccgggaggtggaggttgcagttagccaagatcatgccattgcactccagcctgggcaacaagagcaaaactctgtcgaaagaaaagaaagaaagaaagacaagaaagaaagacagaaagaaagaaaggaaggaaggaaggaaggaaggaaggaaggaaggaaggaaggaaggaaggaaggaagggagggagggagggaggaaggaagggagggaggaaggaaggaaggaaggggagaaagaaaacaaaggctactatgaacaaattaacagctttaatcacaasagcataaatatatatgggtgtggctcctgtatgtgggtgtgtgtgtttcatagcctgagagctaactggctatgaaacagtttctaaacaggtgagaaaacaatgcttggtgtgggcctgggtgccttagaccccaaggtcaggacaatgcctgcacttagacacaaagatacaccgttgagaaggrcaagtctaagatgaaatccagcccatgttctcatcactaagtctttcaactctcttttacctctttgcctggaagaaagagggtacttttactaattagtttgtccagactggctgccatatagtcttttttctctagtacaaaggcactcctatgccagccctggcccataagctctgaccctggagtcagacagacccggctatggattctagccctgtggtgtggtatttggcaagttaacttcacctctctacactgcagcttcctcatctgtaaaataaggaaaacgatctctctcttttctgcatagggcgactcccagatgcaataggactccctttaccttcatggycttcttgaagctgtaatgaggagataatccctggtccccaggctccattcgtatcttacagaacactattcccctttcaaataggtagatttgcctctggctgggtttaaatcgaatcaaatccttcattttataacgatccttgtgaattgtccagacgctgaaagggccgtgcagcaacagcttgcctagttttccaatatcgtccttttggaaacacacatacaggaaaagaagctgtaaaattacttgacagagaaagaagcctttgtggcctgttttcaaattttataaagcatgcttctttgaggtttacaattctaaaagtgctattcaggctggtctgtcactaaatcaaaacgacaattttttgagcttcactgagcgtggtgccrtttagttatataaggaaatcaattcagctatgaaaggtaatgctatacataaagaatgttagaaatgcttgagctagagatacggggactgtgatatttttgactcaattttaactagcagaattcaatttcctctgtgctatgtttgatacatgacagtctccttcaaagggacaagaaattaacatttcaattatgtccagtagagatagaattttggctgcattgyggacccatgagcatctgtgtgttccatctgcatcctaacttctgcttcaggtatatgaggacctgagaaaatgaagcttgttctgaaaagagaaagtttactgtgatctaatcttcacaacattgtaatggaaatagtaactgcattcaactctgagtttttcagagtgagtcaacagtgccaagataataccaaatgacagatttttaagaagtctgtttgactctaggcataaactgtgatgcccctgtctccaccagcaaggctgcctttgtcatatgtctggttgaaagtccaaatgagatgaccctgccttctctgagctctcttcaaaagacagtggaccaagatgagctgggagcgaggcattttatctggtatttttcaaaacttcgattgcttaaagcagataagaattcatttccctgaataaaaacaccaaaaggaacaaattatttgaaggggaccaagacaaagaaatctccctaagtgacctctagctgagatcaatccctatgatttctgagttaatactggaaatatgtacttcattcataattgattgggcatttttataakttattgtttatatgttgggcaaactctaggtatcttttttaaaaaggaattgactttacttctgaaacattaaaagaaaaacttcaagagcaataaactctagtcatttctatggtgtctatggcgttatttcctatgttaattttctattttcttctttggaaaatttacatatgtagactgtggtgaataaaaccctcaaactcccagccacgaggccttctttcctaaaagccaatgctcctgttctgtgttaacagttcaccacattccaaaacagaagggtggataggtccatgaggaccctggagaattttttcactctatctgcatccatcaaggacccactcaaatgtaatcactacagaccttagtgactgctttttcctttaaatccattcaattcataatcaatatcgacataaatagtgcaggaaatatgttttagtaaaggcaactggtagaagtagatacttaatatttgccacataaacaaatgttatcagcacaggttgagcatccataatttaaaaaatctgaaatcctccaaaatctggaactttctgagaaccagtatgacactggaggtgaaaaattccatacctgaccacatgtgatgggtggcagtcaaaacacagtcaaaattttgcttcaygcacaaaattattttttattttttatttttttgagacggagtcttactctgttgccaaggctgcagtagagtggcacgaccttggctcaccgcaacctctgtctcctgagttcaagggactctcctgcctcatcctcccgagtagcttggattacaggcgtgcattaccatgcccggctaatttttgtatttttagtagagccagggtttcagcatgttggccaggctggtctcgaactcctgacctctggtgatctgcccacctcagcctcccaaagtgctgggattacaggcataagccactgctcccggccttcatgcactaaattatttaaaatatgtaaaattaccttcagtctatgtgtataaggtgtcatgaaacataaatgaatttcgtgtttacacttgggttccatctgcaacataactcagtacgtatgcaaatactccaatcctcccctaaaaaaaaaaaaactccaaaacacttttggcctcaagcattttggctaagggatattcaaacttgtagtttctatcttacaatgtattcactctctacctcattttaaacattcgacgcatcttacagaaatatatacagtatagacagatagaaaataaccgagggaattaaggtgaagaaaaaataaagaagttgggagcttggcgcctgcctgtagtcgcaactacttgggaggctgaggcaggaggatcacttgagtccaggagtttgaggccagcctgggcaacatagggagatcaccatccctaaatataaagacaataaatcagatgaactgttccagaaggtgtagaagataaaagaaaagatgaagaggttatatgtaaaatgcatataataaagccatktaaacttgatgtaaacttggctctgactttctaatagcaatacagagggaaacatggtgagtggtacgatttattatgtctgaagacatacacaaaccggttgttcaggaaaagcacagctattcctactaagacataagaaacatttctcccagtgatatgttgtcactgagcaacatcctcaaccacgccctatgatagaaagaggaacatgttttaggtaaagctgcttgtaagaacacatttggtcagtgtggaggctcagctgtcaaagtaattctagaagaggctaggatgataggggaagggggaataagtcgaggtgtacactctctaatgacttgtgaagatggggtgacgtgagttaaggcatccacaaggctattgtagaggaaacagccccaaccagaaatgctgaagaggcagcctcaggcagcaaggtttgtgctttattacctctgaactttcctcatcaccccatccacagccatgctgctgcaccttcataggcaagtaatcagaggatgtgaatccagagaggtaccagcagctgaccatagggcccccagcataaaaacaatgtgccagggktgggggttggcaatgtgatctctccctgaactgaagaaaacaggaggaagtgrtgaccagcaggagayggggctgtgagatgctttaggggaaagcaggcagtggaataaacgagaaggaaacagaactatgaagaaggtgagtcattagaaattatgaggaagaggaagaaactgacggaagggaaaaaaacagtcagtaggtggtaagagsrggggcgagcaggcatgtgggtgcacttgacatatacaggcgggtcacacaggtaagaaccccggctccctccccaggcccaggccccagaggtgctgtggatcctgggtttgctcagttcccatctccgcgaacttggctttgccatcgtgacttcttgagaagctgcaggtcagctttccagtaagcccattattactggaagtgactcggggggagtcgctgggtctttcaagtctggcagaaacatatgggcacatatcctctgggtcatgttgcacaaatgttctcaggaccacacatatgacggtattgcccatctccacgcccaaggtccgatctccagcgggtgagcacatgccctacacggtgctgaccaagggcaggcacacacactgttcactgtgctgtgtggagctgctccgcctccaacaggaagctgcagggacattccctgagcaggagcaagaatgaccttttccgtcacgtgtaccttttctttccaactagactgtagttgatctcaaggtcggagaccacgttgcacaacctctttcagatacgtgttcaccagtatttatttatttattggcattccctagtatttatttatttattggcatttagtggtgcttgggagtaggggacaggaaacaaaatactcatcactgtgagggaatattgatttatccaaggagttcattcagcgacgcccctgtactgggttgaatagtgttcccccaaaaactcatgtctatgagaccctcaaaatatgatcttattcggaaagagggtctttgcagatacaattggttaagatggggttgccctgggttaggatgggtccaaatccaatgactggtatccttataaaaaaagaaaacaaagaaatggagacgcacgctgagggaacagccatgcaaaggcagaggcagaaattggagtggcacagctgtcagccaaggaactccaagaattgccggcagtcaccagaagctcagacgaggtaaggaaggattcttcctgagagccctcagagggaacgtgggcctgctctcaccttgcctttggacgtctagccktcaaaactgtgagggaataaatttctgttgttttaagtcacgtactttgtggttccttgcgacatcagccctaggaaacgaatatacttccaactgttttccaccagtaacaaagaagtgtggccatgtcacacggtgtgtggaagaacctgatggttgtgaactcagagatggaacatagcggctacctcaaatgagcgagcgataagttagatggcctcctctgtacaagaagtgatagcaaaatcaattagtaaatgctaccactttcacacctgggcattctgcaggtaagagccccgatacaaggatttcaaatgtgactacattggccaaatgaagagtctggctgagagagtgaatctagggaagctggcctcagcaagagcttcttttgggtgcccgtgtttccattccaggtagtaagagttaatatgactgtcacaaacttactgcacaaagaagcaggaaacatctgtataaatgcgtccacacagagcctgagtccagaaatgaaaagcagaagctgtgggagcctgtgattacaatagtcaggggcaaagaggtatcttcctttccagcttgagcaggtccccatataacgtaccactagggctgtttattcggcatctgttgggcgccaggcacagtgcttggtgccttacccatcatttaattctcacttactcccggcaaccgcctgtaatataggtattgtgggggggtggggggggtgggggggtggggaatgaagaagatactttgcctgggtcactaggattatttacttagccctatctctgcagctaacactgtggtaaggaaagaggaaactgaaatagaaataaaatcctggccctagctcacaatgaacttacaatacaagtacatcctttagtggtttatataaatttgttccaaaaaggaagactgtttcattcaatctttacaagtcaactttggaacttatccctggcaagtcagaatttaaatgcatctctaatatgcattggatttgaaaaaaaaaaaaacttttgttttttttttgcaaatagatagaagtaaattataactcagtgttccacaaaacaaacttgtttggaaaagaattttaaacttctgtgattaaattagacccaggactaattgagtacacagaaaaacaagaaaatataaatatcagaagggatttttttctagttgctttctagttgtttaattgtgataatttaattatcacattcaatttctttcctttataaaaccaggttaatattaaatttcaagcagagaaactaagaggttaaactggactcagccatgcatatagctgtaatttattacaggtgatcataaacacctgtaataaaactctacaaaatggagacaaagaatctcagaatcatttgagctgagttagaggcagttgtcagtcaattctaatcagaaacagataaagagtggaatagcaccctgatagggaatgaacaagaaatagtccaaggtgacagccatgtaatcttgagtgagttaactcttctttccatggatcttcttttgaatcagaaggttgaaaatnccaatatctactcctncataattcaacaggattatacaaaactgtcaggagaaattagctgatgtattagatgtaaaagtaagttaagtcttagtagaaaaggtaccaaaaagcatctggcagaggcacaattctgaggacacatggtcaaaaaaggtcctttgctttttgcgtgtaaaatctccaaattctgtaagctggttcatcctatttgcatgcaaactggctgggaatgaaattagggcagratgttatttgctcatgttttaaccattcttttctcacagcctcctcctcctatgttatttacactgtttcctgccctgtgtattgtttccaggacattcattagattcagggaaatgaaatttaatagggatgtctaatacgtaattcaagatttaaaaaggaacagaaagatgccctggattgacctaacaaattgttcccttgactttcctcagscgaagaaagaagaattattaccaagaaaatgatccttatacacaccctaaccctactctgcagtttatgcatatcctcttgaatcatgcatcagttgtcatcagagacaccttggagtcccaggggacagtaacagcatcatggctgggatcacagcaaaccccatggggctaagtttctgagatccaggcccaagaaataccagcgtgggaagtaaaaataactcatttttcggaccttaataactttcatggtttgatttatgacaaggaaagaaaagtcatttcctctaccaagacctcaatacatggtatgttcaaaaccaaataaaaatgaaaagaaaaaaaaagagaaggaaaaagtataatataatcacaagtgacaaaaacgcagaagagggggtcacagagatgtgatgtaagaaaaacttggccgggcacgatggctctcacctgtaatcccagcactttgggaggcggagatgggtggatcacgaggtcaggagttcgagaccagcctgaccaacatggagaagccccgtctctactaaaaatacaaaaaaaaaaaaaaattagcaaggtgtggtggcaagtgcctgtagtcccagctactcaggaggctgggacaggagaatcgcttgaacctggcgggcagaggttgcagtgagctgagatcatgccattacactccagcctgggcgacagagcaagactctgtctcaaaaacaaacaaacaaaaagaagaagaacccaacctgccattgctggtttgtgctttattacctctgctcatagaagccatgagccatggagcgtgggtggcctttttaagctggaaaattccaggagacagatgctagactccaaaaaggaaaggaaacaccttgattttagcccatgagactgtgttgaacttctgaccttggaacttcaaaataataataataatgaatttgtgctgctctaagccaccaagcttatggtaatttattatgacagtgaaataaaacaaacacatctgaataaaaatctagagtacttcaaagtaaaaaacaaaacaagtcgaatattaactatgctccagtctcaaagagatcatacagttataggggatgttttgcctggaatgcctgggaccagcctgctccggattaacactgacgctggagctcaactccccagcattggcctctaactgcatctcttgggttttctttagactatttggattaatgcatgtacactatatacttttggggcctcttctgggatctgtgatataaaattggggcaagaatataggacctgtttccaaaatacttttcttagagaaactctgaaacaagaagacaaatcttttactattttaatagtgccatgagttagaaaataaattgctcctccaggacttttcctctgcccatatatgttctggcccttgactttctggtttgtccagacagtgggtcagtcccacattcagaaccctgaggaataaatycaagattcctgaagatgccaagggacagcttggaatcttgtagtgcgggtttactgagcaatttaggcgactgtcctcagagtccaattcctttaccatagacgatctctctgactttgccagagcatttcctcactgtgtagtattgcacctgccttttgtgtgagatgctctaacactactatggaacataccggacattcagtcactgctgctaggtccacagccaactcacaggacttgatcaaatcctctatcactgccaaagcttgttgtagttcagttgagaatgctgaatctttggttctctttggcccatcctgtggaaaacaaatgccttgttggaaatcaaaagtataccatctgcaaagtgaagaaggaaaacaggacagagatgaggagccagggatccagccaaccctgactgagaagggtggatattgatctgtctccctgctcccaaagcatggtggctaaagatgcttactgatttaggtatacggcatagagacaagcaaaatcatctacctctgtgcattgtactagtgacgaatgaagttcataaatcatggaaattatattcctgctacataagaaacatatagaattcagctatatagctgaattctatatgtttcttatgtgtatatatattatataaatatttatatagtatattaaatatttatatattaatatataattatatgtttatatattaattatatatattaaatatttatatattatatattatataaaatataagtatatatattatatatatatatatatatatatatatattttttttttttttttttttttttttttttttttttgagagcgagagagagtttcactctgtcacccaggctggagtgcagaggcacgatctcggctcgctgcaacctctgcctcctggattcaagcaattctcctgtctcagcctcccgagtagctgggattacaggtgtgcgccaccatgcctggctaatttttgtatttttagtagagacggagtttcgccatgttgaccaggctgttctcaaactcctaacctcaggtgatccacccaccttggcctcctaaagtgctgggattacaggcgtaagccactgtgcctggccagaatttagcttttttaaagcatggaaaaacctagctccttttaaataggcttctctctcttttatacccctattccctggggcttactgaaaaaaaaaataccatatggccgggcgggtggctcatgcctgtaatcccagcactttgggaggctgagacaggcagagcacttgaggtcaggagtttgagcccagcctggctaacatggtgaaaccccgtctctactaaaaatacaaaaattagcttagtgtggtggtgggcgcctgtaatcccagctacttgggaggttgaggcaggagaacagcttgaacccaggaggcggaggttgcagtgagcccagattgtgtcattgcactccagcctgggtaacaagagtgaaaccccgtctcaaaaaaaaaaaaaaaaaaaaaaaaaaaagtagctgggtgtggtggtgtgtgcctgtaatcccagctactcaggaggctgaggcaggaggattgcttgaacacgggaggtggagtttgcagtgagccaagattgcgccactgcactccagcctgggcaacaaagtgagaccttgtctcacaaaaaaagaaaaaagaaaaagaaaaagaatacaatctgagtatcatattcctatatttaatgtggaagccttattcctgtgggaaaaattatacttaaaatcatcccagaggaggacatttgtaaactcctatagagacaaagaactccatagaggctgctaagtggaaatttactaatgatttacatgtaaaagctataacatcatatttccacactgaatctcccccaactctgtccttccttcctccacttgtccctggccctccccacattgcaccaccattaaagatgccaaagagataagccagcgctctgcacctcccgaacataaagactcagcattcagcrgaaaagcagtaactaattaaagagaccaatgttccaattacaaccacagtgacatttaggatgtgattggggtgattgtttcagctctaaaggcttttgcatgggcttgaggtattttatctccctgctacctacatgctgtatttatctgttacctggtaaatacacataaaaaaattttggtttattcaatgtatttttttaacatctcaagttctgcagtgaagaacagctagcccctttgctgctccgcatctggccctgactctttttgtcctctacagcacaatgtagtcacagggtttaatattttcttaatctatgcggaagcactgggtatttgcatctttggatgagagacatgagtgacagggctgatgaatggaatagatcgtgctgctgcctctgaaaaacgtatcatggattcgtagcttctcattcataataaagaagcttcgcttcctggtaaaagaaaacagactctcacctgaatttattttaacaacatcaataagggattaagagttcttggcagggcgcagtggctcacgcctgtaatcccagcactttgggaggccgagggggggagtggatcacgaggtcacgagatcgagaccagcctgaccaacgtggtgaaaccccatctctactaaaaacaaaaattagccgggcgtggtggcgggtgcctgtaatcccagctactcaggaggctgaggcaggataatagcttgaacccgggaggcggagttgcagtgagccgaaactgcgccattgcactccagtctgggtgacagagcgagactctgtctcaaaaaaataaaaaaataagaaataatttttttttaaagttcttataccaggcatcttcctccttaccaaacactgaatgctcagtttcctttcagatcctcacctcttcccatcccccttccttccgctaaccctccttcacaaaacacgaggtggctagggttttgaactgtataatgtagggttcagaaaaatcctttaaaacattattaaactcctctaactagggctagctcctcttgctccaggctgtaaaaatatgacctgtgtcctgagctgcttctgttttcaacaggtccttgtcatccattctgagcagaaagggcatgcaaatgactgccccataagacatgtctcaagtgtttcttgctaaaaccagaatattctataggaaagggaagagaaaccgcactgctatamcacagatttcttcctaacctggtgtggtcatggtcaccatttattctaagggaactttggcagactcttagagttcacacacacgcacacacacacacagaggagaacaaggcataacatatagaaattagatatactaagaggtacaaagaagaaaacataatcctaccattcaggaaagccacagcagacattttatcatatctatttacttccagtcttttatgtatgcattttacgtatttgcttaattttgttcccattaaaatttttttggctgggtgcagtggctcacgcctgtaataccagcactttgggaggccgaggcaggtggatcatctgaggtcaggagttcaagaccagcctggccaacatggcgaaaccccgtctctactaaaaataaaaaaattagctgtgcatggtggcgggcacctgtaatcccagctacttgggaggctgaggcaggagaatcgcttgaacccgggaggcagaggttgcagtgagccaagattgcaccattgcactccagcctgtgcaatggagtgagactttgtctaaaaaaaaaaaaattatgtggaaggaagaaaatatattaccacttccatttgggctgcaaatccctactttaaaattgcctttaatttttttgttttttttcttgactgtgagttataggatacatactattttagaaaatctagataataaagaccaccaartaaaattgctataaatcctccgamccatagacaaacaccattaatagttagttatataaaatgtaatatttaatattgagtaccttagccgagcctcctagttcacctgggtctatctccatatcttcaggagactcgaratccagcagaccctgcattaacccaaaagtcttgttgaaaattaaaatgtacattgccatcaaaatgtagattgtcaacaaaatctactgtgcaaactatctgccaccaatgctaagctgactgagaacgtgtgtaaatgtgtaagagggaaagaataaatgatgtatttggcacagggtcctttccacaaacctagaatcatattgtacacactattttgtagactactttttctcattcaaccatacctcatgaatattttcccatgatattatgtttttctaaaatatgggctttgattgtggtagagtgttatatcttacagataaaccatttttctcattcagcaatatctcatgaatattttcccatgatattatgtttttctaaaatatgggctttgattgtrgtagagtgttatatcttagagataaaccatttttctcattcaacaatatctcatgaatattttcccatgatattatatttttttaaaacatgggctttggttgtggtagagtgttatatcttatagataaaccataatcgaattatagtcccccttatcattaaacatttaggttcaacaatattttttactataataatgataataacttttgtgcatttttcttaaagctaaattcctagagtggaattgtaaatgttaagagttttgaggcatatttccaagttcttcagagaaggactgtcttcctgccagctgtataggaaagcctgtctcaccagcccctagcctacaaggggtagtaccatttaatcaaaaatctttaccaattgaatgaaaagagaaatcctacctcactgttgttttaatttgacttccttagataactagaggtgtagaacttttttaaaaaaacgtttgctactagtgtttattcttttgcaaattgctatgtcctttgcgcatctttttattataatttccaagagcacatgattcattaagcatactgatcatctgttacatattttgttcacattttccccaatgtttcatgagtctgtctttaatggtataagctatcactcatcaccctctccccaagatcccatgatcctttctaaagcatgaggcaatcagtccaacattcatgctctttcaagccagcacatgtgtgcgagatgcaaaataagctctgcccctggggatagagaaggtcctagataggattacaaggtygttcctttctttgcaggcacgtaatggcctgagctggtttcacaggcaccagcaagcttagctgtggggacactgctctggtctgccttgggtagctcccacggctcctcacagaccccccgcaaaatattttgtagtaattctatctcttgtttcttcagtgaaaggcagctaacaaaaccttctaagtccttttaacctgtattatctcatttaatgctcccaacaagcataccaggaaagtactattattatcttatttctataggtgagaaatctgaggctgagagaggttaagtaacttgcctgagttcacgcagcctggaagggtcagagcctggattcagacccaggttgttaaactctcgagtctgtgtttccaatgacgatgccccacagccctctgcgtctggtaccgaacctagctcctatgtaaatgtcacctctgtgggaaagctggaacctaggctgagggaggaaggaccatcactttcgtcctgctcatgcctcactgggcccagaggttggacttctacaataggaatgacagtgacaatgggacatgcagaggagcatgaggccctgagtgagtgctgggtcaaacatgctctgaggtgctgctcgctgaaagaagcagatcattatcctcatttctatggtggtgtgacagtctcaatgtctaagataacagctacacatcatgcagagcttcctgggctgtgccaagccctccgcatgtgctaatcatctgcttcataacgatcttatggaggaggttctagtaccctcattttacatagaggaaaactaaggcccaagaggkcaaggtctcataactgacaaatggcagagtacaccgaggctgaaactgctaatggaaagcttgaaaggagtaactttgaaaaaatgtttttaaaattatttttacagatgaggtcttgctatgttgcccaggctagtcttgaactcctggactgaagggatgcctcctgcctccacctcctgagtagctgctgggactacaggtgtgtgttatcatgtctggcctgaaaggggtaactttttctgaggggagactgttgggcaatgagcttaaccttcctgagcctcagtttcttcacagtccagagcatggctggtatgtctgattgtggagattaaataagaaatactgtaatcccagcactttgggaggctgaggtgggcagatcacgaggtcaggagttcaagaccagcctggccaacatggtgaaaccccgtctctactaaaaatacaaaaattagccaggcatggcggcgcgtgcctgtaatctcagctacttgggaggctgaggcaggagaattgcttgaacctgggaggcagaggttgcagtgaactgagatcgcgccattgcactccagcctaggcagagtctcactctgtctcaaaaaaaaagaaagaaagaaagaaaaaagaaatacacgtggggaagtacccagtaaacagtaggtgcttgtttttgtttttgtttttgttttttattaagaaatagtggccactgggcaagagctacctaaatgccttatagaggttaattcaacagacaacttatcgtggtggcccaaacctttctctcaagctcacacctagaaaaccagccatgataggccctcatatcctgtttggagtgtctgagaatgcttgcctgcagatgtgctagtgtcaatgacttatcactaaggaggggtaaaggtcagagagagagagagaaaaccacttaggggttcagggttcagatatagcaaagttcattctgttaggctgctctcagggtaagatcctaaatctaagaatggaggtcagtgctgcaaaaggatagaagacttcagttttgtcttctcccttccacaagttaaaatgcccataaaacagtaactttggggaaaatcacactctcgctcccaaagagctctcttcccctaagccagactccttagtgattcatgccctgagctagacattggactagtgaggccctcaggtgccctccaactctatgattctttggtcttactccacattgaagagggctgatttagagttggaggaaagaaaaagcctgcaggatagacgtatcttcagccttagaagaccaaggcacagctctacatgtgtaacgacatgggcaccacagtgcaggcgtctggtttcccaccaaggaaaagcactgtctcattgattttaccttcaacagcatctggtattttataagtctctggcttggtcctttgagatacttaaaaagagggagattgtgatccagttggcgctggcatacctttaaaagccaaatagaaacaacataaagcaaaacaaacaacacacacagaataaaagtggcagcatttaggtacaaaacacaaaattgcttttcctggacaacaggggcagctgtgtcgagcagctccataaggctgtggggttgttctttcccctaggaacagtgtgtgaaataagagaaagaggaaagaggacggctcggctaatatttttacaggcatctagggaagcagaaccatttatttcttttagaaagcaagttcactagaagacagtggggcatttcaagtccttctgaaaaagaggccacctatcttcaagttgggggctgccagagtctattcaggccttttggagcctgaaggatacagacggtttgtagacatcattccagggagcccaaagtgtgacattttttacttagtttcatttttaaaacccatcttagaagaagacactggttttctcaccagatataggtgctcctcacaggcctatcccattacaacttttcatgtgtaatttcaaagtatgggacactttaaataaacataaatcgcaatgccaagttcctctccctagtcccacttttgatttctttaacaacttgatgcatagaaacattttccaggaagaagtttgtcttctcaacattaccccaaagtaggcgcagtcttgacactcttgccagattgccctagctcggggcagattcttatggtatttaaaatatatctgaagatcttctttctaggtgggaaaaaattagaataaattaatcaggcagcatttttcagtaagtggacaggatgatacagtttattcattcattcaacaagccagagtattctaatgtgtaaggaattgggggtgtacagtaagtaatcttatgttgggatgtgtgtgtgatggagagataaacagaaggctttaaacaaccataactaccagacatagataggcacagagtgctctgggagcatctacaagggctattggtaaatacataagtagactcacaaacaggaattggtttttatctttatcattcttttcctgccctttgtccaggacatcgacatctccccacaacaggtctgcaatgagtagtaaaactgacagaaaagaggctggtgtttgatggtccccctctcttctctctccaccctctccttgtggtcatcgtgacaccaaccctgcaaagaagcccaagaaacaaccaaaagatggaaacatgccagaggggagatgtggtagatcgggcaaccaatacggagctcagtcatttgggcaaccctcaaagtgacaggctccgcctcaaacatcactttcaacctgcagcacaagactctgagacaggaaatgttatgtgactgtttcggagctagagagcaaggaggactcaacgtgctgttcaacctgtattctcatggtatcaaagacgctgagaggtgggcacatatctgccaatctcccggagagaaagcacactccaagcctctggcgtcagcttgccagcttcactcgctctatcagggagcctggcccacagcggctgtgctggccacagatcccatgccattctggtggagaagcccatgtagccacctccttgcagaagtctgtgtccctgtcctcctgctcagcccaactctggatttccttcatgtcaacacagatgattccttccctctcagcttcaatgatgcctgttctactcccaaatacattgctatagtggatgacttccatttctgtagctctttctttctcccaataggttgtaagtggtcatttcagcataggaactacagcaaccgtcctttgctattccctccaggaaaaaaatggcacacacatttaagacagatggcccttgaaaaatgttcagactggttaaggagctagaataataactcagagcttaacaatatagtattgacctaaattgctcttctaaaggctaacagtccaaaattcctcagagaaaggatgtccactaaccagcaacatacaaaagtgcccaattctcctaatctccttatagaatgaatcaggaggctataaaattgctaccagaaactcatattcacacaagaatacatacttgcttacggattaagactgcggggctctggtggcggatggtccaggttcagttcttggctttgccaaatacaaatcatatggcctaaagtaagttacctaaccttcctagacttcagtttcctcatctgttaagtgggcccataacagtagtcaactcctgtgactgttgtgagattaaatgagatgatgtatagaaacctctccacaaatgcccaatggatagagtactcaaaaaatggtggctattgtcaaagcactttttactttatggaatcctctgcatacattattttgtttcccacaacaactgtacatattggtactattagcctgttgtgtggaggaggaaagtgagattccaaagtttaagttatttactggaggtcatgcagatagcaagtggcttgaccaacatgataaatcgggtttcctgacttaccagttactcccacatatcagtgtgagcacccattccagtgctctttccattttaccatgtgagatctcagagtacttggaacccacagtacactatggacaaatattattctatatagaacatggtctctggaggagaattgtttcagaagatacaggctaaagaggctgattaaaacacttggtcagaaaactatttcaaaattcattatcttggaaggccgaggtgggcggatcacgaggtcaggagatggagaccatcctggccaacatgatgaaacctcgtctctactaaaaatacaaaaattagccgggcgtggtggcacatgcctgtaatcccagctacgagggagggtgaggcaggagaatcgcttgaatcagggagtcagaggttgcagtgagccgagatcacgccacagcactccagcctggtgacagagcgagactccgtctcaagaaaaaaaaaaaaagtactacttgtattttgtctctaacatcataaatcacatagggctaagtcagtgttttctggctgggttaatgatttacaatgttcctcccaacatggcggggcgctatcaaaaaactttgtaaaactttacaatttaggagaggaattcagaaaaggtatttagttaataattctctgacaatttcctcatatttgaacaatttgataatatccactttcccacaggaactttgcccatttctcattgagcaatttaaataatctttgcagtaatgacatacataaccataaaatacttctaatctctgaaactagatttcacattagtcgtttgttaagtgcaaaaattcaaaatagtatataaagcactaataatgtaatagttcaaaaaaattaatgaatcagaatgagtataaaatagttttcagtgtacattgaattctggtgctttgacacaaagcctatgatgagttgacatggaaagccatatggggccaccaaaaggctctgagcatggaagattccttctctccattaatgtcaaatatcttgaaacctggaggctttgtttgcttccagttatcataagtattgctctcagcctcagtcataatatgagttgcttacaactttaagttgtttgatctgttgccttgggttcacaaaaataaaaataagaaagagaatgagaaggactagttgagaaagagaggagaaatgagaactatttagaaggcacagggggtcaggatcatctccatggtcgtgtcctagaaagtacctggtggggattgatgttgggtggaacatttctctgtggcagctcccactggcccatatagcctatttgttgcctaaaacagggtggagaaacagggaaagagggaagaagtctgggaggtgggaagaaggggtagttggaggtataatgttgtaggccaagatgttcataaattgagtactaagcctaaaggaggctgtagatttagaattttcgaatcatcctctatgttgtaaaaaatagaatggaaagaaaatgtatttcctgaggtttttagtttaacaccagtgcttctcctcttgagagcattagtacagctttattgtgggactctaacctcgctgtatgattcttgttttaaaacgtagttgagtcattcagaagaggatcctctgctttggggaccatggatggccagccttccatttcatggggctttgcaggaagccaggcttgcggttactcccacatatcagtgtgagctcagtcaaattggactcattctcaccttggataaagtcagataaagttgggaggccaactcatgttttccattcaaagggcagatacaagatatactgtcttgacaaattgcaagtgtacagtacagtattattccatgatagtcgccatgctgtgcgttacatctccaggacttactcatctcataactgcaagtttgtaccctgtgaccaacatctccctacttcctctagactccagcccctggcaaccacccttctacttcaatgagctcaactgtttttggattatatatatatacatatacacacacacacacacacaatggtaatgaatgtattcattaacttgattgcagtaatcatttcacagggtgtaagtatatcaaaccatcatgttgtatagtttgaatatatgcaatttttatttaccaattatacttcagtaaagcttggagtcggagggaagcagatatgtttttagaccattgatgtttccatttgtccttgccacttaacgcagaaatgtaagggctgactttaacatgattatttactgggagtacagacttactctcttgagaaagcaatgtgccagaagttcagggttctcagcacacttttccaactcttttagaaaagtcctagaaaaataaaagtatacaagtaataatgttttgattttgacatgtaggtaattaattttttaagtacaatcacataaggtttcaagaggtgcctagaaaaatgttctccaggccttcttgctcttatcttctacacattgattcagraaaaaaagctgtattctgaggtattactacaattaccctgaaatcactcacaactttaagttgttcgatctgttgccttaggttcacaaaaagaaaaataagaaagagaatgagaagaactagttgrgaaagagaggagaaatgagaactactcagaaggcacagggggtcaggatcatctccaattatcagaacacagctgttcgggtaattctaagcactagtcatagatagactggatacattcactgatgctctctcttacatgaaagtaacgttcataagaactgctgctgctgctgttgctattactaatatcattgacagcttactataagccaggcataagctaagtgctccgcaaatagtatctcatttaatcctcaaccaaagatagggttttataaatataaaacctggggctcaaagaggctacataacttctctggggaaatcaacagaaatgggtttccatttcagttttgcgtctgaccaacggacgaggaatggggttaggaagcaactgtggttcaattcaccaagcagcttttctccctctgtgaattaggtgtgcaatcttggggtcatcatagcgaataaagagagttaacaagttaccattcccatatgtgtaacatgaaagggtttgaagatgatttccaaagtcccttattcctgcaattctctagaactcaatatccaagcagccccgagttaaaagtacaacttgtttgagcagtcagcattttctaacaccctagttcccagcatccccttatgatagtagtatggagatgtgtatcttggtcatctttgtattcccaatgctgagcacagtgcctggcacagaacaggtgctcaaaaaatgctgatgcatgaataaataaacaaaggaacactcaactgcatacaacatggatgtctgacactgggtccctgttcctttgatgtctcttccctttctctccagggatcctgtggggcttctcccctcctttcactccaacccaccccaatccaatccactgactccaaaccacttgcttaaacctgaaaacatctaagtgttttcatctctcaattccatcagtctcatttccacccttctsttactctccattctttctgataacatgaattattatatacctgttgtgaaattcgtaaagttctctaatattcccaaagagaaagtccttgttattctgaagaacatctggaattagatgctttagccaaataaaatccattggagtgatatatccctgcagaggtgcaaacaaggtaggtgttacaaacaggaacataccagagatcatctgaattagctactgcaaaacccaaaactttagtgaaatgattgaagaatatagcacacttgtttcttgaaagctatcgtattgagtactttcttttgatatcattttcctcatgtgcccactttcagaaggagatggcttaatggcaatgatcataaaatggcatttttgtgggaagaacaaatatatttaaaaatgtttatgttacacaggtttaatctaaaagaagggaaaatgtacccactgttttatgaactaatacaattactattattattatgattattattaatattgagacgaagttttgctcttcttcccaggctggagtgcaatggcgtgatctcggctcactgcaacctccgcctcctgggttcaagtgattcttccgtttcagactcccaagtagctggaattacaggcacacaccaccacacctggctaattttcgtatttttagtagagacagggtttcaccatgttggccaggctggtctcaaacttctgacctcaggcgatccacccgcctcggcctcccaaaatgctgggattacaggcgtgagccactgcatccggcccaataattattttttaatatctgaataatgtttactcagcatatatatcatagtttactaaacattccctttttatggacatttgggttgcttctaatattttatggtaaaaatgtgattataaataatcgcgtacatataattttttccttaagattattcccttaggaaaatagggaaaacatgcatttttactcttaagaaaaagactactttaaaaaaacaagaaataatcttcccttattaaagtatcaggaacacttataatatcctttgttggcaagaagatggtaaacctatttgggctttgctggaatcagtgtgaactggcttaattctttggaaactatttggctacatggatcaagaaaccacagggatgttcttattcctactggagacacttgtaatattctaagacctgaaaccttggtgggaattctggagacttctggcaactattttgagtctctttgtacaacaaaacatctgcagtttgaatatatgcttgataccccactccccaaatctacttacatcaattatgcttttaatctcttttatgtaaatctcttcagtctcaagcaagtcacgtataatgcgcctgaatcacagcagcaggtgggagggtgaaagagagagagacagggagaggagaatgttcttttagaattctgttagaataatgctcatcaatacagttcccttttagtggctcacctacttagtttctggtcagttcattctgttctattgaacacccaaggtcagcatctcacaaacgcacactgtgatcacactggtatcaagaagaaacagcaaggttagagaactcaaaatcctttaggcagccagtgaatgacctgtcctctgggtgggccatatagtgtgggggtcaagaagggagatgttggagtcagaaataccaggtctggatccttagtcaaggaaggtctgcctgaccccacccccaccccctatattgtatacattgtataagtggtttcctagattttctctccagctttctgctcacatcaagctttcttttccttttaagagaaagggtcttgccctgttgcccaggaatgagtgcagtggcatgatcatggctcactgtagccttgaactcccgggctccagcaaccctcctgcctcagcctcccaagtagctagggctacagatatgcaccatcacacccagctaacgcctttttttttttttggtagagatgaggtcttgctatgttacccaggctggtctcaaactgctactttcaagcaatcctcctgcctttgcctcccaaactgctgggattataggtgtgagacactgtgccaggccagatccagatctttgacccatatggatgtattttgttgtacatggggttagatgtatgctagctccttcctctggcattggatgtttcactgtgatttcctaaggcaaaagatatgacttctgtctgcttctgtcaaggaatgcaggtggaagatgtggtggacagaatttaagacggtccccaagacttctggccccactgcacatacgcctcttccaatcaaacactaatttaggggatgctgtggaaggattttgctgttcatttgataggtaatccagttgggcctgtcctaatcatatgaactcagatctatmtgggtcaagtggtcagagactggaagcataaaaaagattcaacacaaaggagattcccctttgctgacttttgagatggagggggccagatggaatggaatgtgggcagccgtaggaactgagagtggctcccagctgacaggcagcaaggaagggaaatcagtcctatagttgcaaggaaccgagtcctgccacaaccacgcgaacttagaagaggatccagagctccacatgaaaacacagccagccaacaccttgactttagccttgtgggactcctgtcccagggaaactgtaggtctcctaagtttgtggtcagtttttatatagcaatagaaaaccagtagaaagagtaaggccagtccccacatctatcccagacagactttctttctctcctcaggctacgtggccatatttattatttcttttaggagtccaagtagatgagtgtttatacatgtgtccttgtgtagaatatatatatttattcttgttaactattgatggattatctattaaatggcagcctctcctagaaaatgatttgtttttctctcataaaaatggatgtgacaaatgatccaatagaaaaaaatgggaaaggacacaaaataggagattcacacaaatattcaaatggatatgagactcgtggaaaaaatactcagtacatacattcaacgaaggcatattttaaaagagcaacaattttcatctattagaatattaaacatttcaaacctcatagaaagactggtaaatgagggtgtggaaaaaccctcacacaatattggtaaaatgtgtaaatttgtgcaagctctttggaaggtaatttagaaacatctcacccaaatgtaaaacacgtgctccttggttcagtaatttcacttctaggaatgttcattgtttataattaggacaatataacttcaacaacaaccagatataaactggttacttaaatgctcatcaaaaaattatggtacatgggccgggcatggtggttcacccctgtaatcccagcactctgggaggctgaggcaggtggatcacttgaggccaggaaatcgagaccagcctggccaacatggtgaaaccctgtgtctactaaaaatacaaaaaattagatgggtgtggtagcgcatgcttgtaatcccagctacttgggagggtgacgcaggataattactcgaacccaggaggtggagattgcagtgagccgagatcacaccattgcactccagcctgggcaacaagagtgaaactccatctcaaaaaaaaaatatatatatacatacatatagatatagatagatatatgtgtgtgtgtgtgtatatatatatacatatatatggtacatacatataatgatgaaataccatgcaattgttaaaaagaatgaggctgactaaaacactgatatgacagaggccaggtatttttagtgaaaaaacaaaacaagacgtagacaagtaagcatagtatgactatttgtgtaaaaaatgaatgtgtatgtagaaatatacgtaaaactgtatatgctcagaaaatttatgcaaagaaacttaacacattgttaaaagtgattgcttttggagagtggtactgaaagctaaagtatgggaaaggagaatttctacatcttactctatacccttctacaatcttttttaaagtgaaacatttactgcttttataatggaaaaatagggtttacataatattttaaaatgaatgttactggaggtaatgatatgtaaaccctatttgatagatataaataaataatacaattcacatattttacagctcattattctttcttataatcatttttgtgtctatttataaatacttgtataagacaagactgataaacaagatgtaccacagaggatgtaatttccggaagtctaaaactcccaaaaagttacttaaacatcatactttactaagtcactgagaaagaggttcctatgccttttataaatcacccggaaacaacaacggtttctatgtcagaaggaagtacaggcctcaataccaatatgtttctacgagccctggcatcaagtgggaccacagcaacagttaagcattcatggaaaacctgagtacaatcagcctcttatgccttcatgaagcactgggcttaagcaacttggcaagctggcttcaggatgcattgacaattaaaggaagtcagaaggcagaaataaccaggaggtggacagaagtcaaggttattatccaaaaatgatctatgaacttaagagactcttaggagcttttaggactctaatacaggaaaatattcacatctctgaaggaaaagaaatcccttggtcatattctggattacagtttggaaaaaaagtctagaaatctcatccaactgcttcattttacctgttggagtgcaagagataaaatgccaaggataggaactccactgaagaatgtaggcaaatcaatattcattaaatacattcaaattaatatatgacttaaaaagagccctcacatcagtgttaggccattgcttttccttgcttctcttgttttcctttcaaatacctttataaaataggagaaaatgtatgactcttttttttttttttttttttttattatactctaagttttagggtacatgtgcacattgtgcaggttagttacatatgtatacatgtgccatgctggtgcgctgcacccactaatgtgtcatctagcattaggtatatctcccaatgctatccctcccctctcccccgaccccaccacagtccccagagtgtgatattccccttcctgtgtccatgtgatctcattgttcaattcccacctatgagtgagaatatgcggtgtttggttttttgttcttgcgatagtttactgagaatgatggtttccaatttcatccatgtccctacaaaggatatgaactcatcattttttatggctgcatagtattccatggtgtatatgtgccacattttcttaatccagtctatcattgttggacatttgggttggttccaagtctttgctattgtgaatagtgccacaataaacatacgtgtgcatgtgtctttatagcagcatgatttataatcctttgggtatatacccagtaatgggatggctgggtcaaatggtatttctagttctagatccctgaggaatcgccacactgacttccacaatggttgaactagtttacagtcccaccaacagtgtaaaagtgttcctatttctccgcatcctctccagcacctgttgtttcctgactttttaatgattgccattctaactggtgtgagatgatatctcataatggttttgatttgcatttctctgatggccagtgatgatgagcatttcttcatgtgttttttggctgcataaatgtcttcttttgagaagtgtctgttcatgtccttcgcccactttttgatggggttgtttgtttttttcttgtaaatttgtttgagttcattgtagattctggatattagccctttgtcagatgagtaggttgcgaaaattttctcccatgttgtaagttgcctgttcactctgatggtagtttcttctgctgtgcagaagctctttagtttaattagatcccatttgtcaattttgtcttttgttgccattgcttttggtgttttggacatgaagtccttgcccacgcctatgtcctgaatggtaatgcctaggttttcttctagggtttttatggttttaggtttaacgtttaaatctttaatccatcttgaattgatttttgtataaggtgtaaggaagggatccagtttcagctttctacatatggctagccagttttcccagcaccatttattaaatagggaatcctttccccattgcttgtttttctcaggtttgtcaaagatcagatagttgtagatatgcggcattatttctgagggctctgttctgttccattgatctatatctctgttttggtaccagtaccatgctgttttggttactgtagccttgtagtatagtttgaagtcaggtagtgtgatgcctccagctttgttcttttggcttaggattgacttggcgatgcgggctcttttttggttccatatgaactttaaagtagttttttccaattctgtgaagaaagtcattggtagcttgatggggatggcattgaatctgtaaattaccttgggcagtatggccattttcacgatattgattcttcctacccatgagcatggaatgttcttccatttgtttgtgtcctcttttatttccttgagcagtggtttgtagttctccttgaagaggtccttcacatcccttgtaagttggattcctaggtattttattctctttgaagcaattgtgaatgggagttcacccatgatttggctctctgtttgtctgttgttggtgtataagaatgcttgtgatttttgtacattgattttgtatcctgagactttgctgaagttgcttatcagcttaaggagattttgggctgagacgatggggttttctagataaacaatcatgtcgtctgcaaacagggacaatttgacttcctcttttcctaattgaataccttttatttccttctcctgcctgattgccctggccagaacttccaacactatgttgaataggagcggtgagagagggcatccctgtcttgtgccagttttcaaagggaatgcttccagtttttgcccattcagtatgatattggctgtgggtttgtcatagatagctcttattattttgaaatacgtcccatcaatacctaatttattgagagtttttagcatgaagggttgttgaattttgtcaaaggctttttctgcatctattgagataatcatgtggtttttgtctttggctctgtttatatgctggattacatttattgatttgcgtatattgaaccagccttgcatcccagggatgaagcccacttgatcatggtggataagctttttgatgtgctgctggattcggtttgccagtattttattgaggatttttgcatcaatgttcatcaaggatattggtctaaaattctcttttttggttgtgtctctgcccggctttggtatcagaatgatgctggcctcataaaatgagttagggaggattccctctttttctattgattggaatagtttcagaaggaatggtaccagttcctccttgtacctctggtagaattcggctgtgaatccatctggtcctggactctttttggttggtaaactattgattattgccacaatttcagagcctgttattggtctattcagagattcaacttcttcctggtttagtcttgggagagtgtatgtgtcgaggaatgtatccatttcttctagattttctagtttatttgcgtagaggtgtttgtagtattctctgatggtagtttgtatttctgtgggatcggtggtgatatcccctttatcattttttattgtgtctatttgattcttctctctttttttctttattagtcttgctagcggtctatcaattttgttgatcctttcgaaaaaccagctcctggattcattgattttttgaagggttttttgtgtctctatttccttcagttctgctctgattttagttatttcttgccttctgctagcttttgaatgtgtttgctcttgcttttctagttcttttaattgtgatgttagggtgtcaattttggatctttcctgctttctcttgtaggcatttagtgctataaatttccctctacacactgctttgaatgcgtcccagagattctggtatgtggtgtctttgttctcgttggtttcaaagaacatctttatttctgccttcatttcgttatgtacccagtagtcattcaggagcaggttgttcagtttccatgtagttgagcggctttgagtgagattcttaatcctgagttctagtttgattgcactgtggtctgagagatagtttgttataatttctgttcttttacatttgctgaggagagctttacttccaactatgtggtcaattttggaattccctgctttattattctaaagcaagcttgtccaacctgcggcctgtggcccaacacaaatttgtaaactttcttaaaacgttatgagattttttttgcgattttttttttttttttttttagctcaccagctatcgttagtgttaatgtattttttgttttgttttgttttgagacggagtcttgctttgttgccaggctggaatgcagtggtgcagtctcggctcactgccacctctgcctcccaggttcaagcaattcccctgcctcagactcccgagtagctgggactgcaggcgtgcgccaccatgcccagctaactttttgtatttttgtagagatggggttttaccatgttggccaggatggtcttgatctcctgacctcgtgatccaccctccttggtctcccaaagtgctgggattacaggcgtgagccacctcgcctggccagtgttaatgtattttatgtgtggcccaagacaattcttcttcttccagcgtggcccaggaaagccaaaagattggacacccctgttccaaagcatgtaattttattcacagaaaagactctcggccgggcgcggtggctcacgcctgtaatcccagcactttgggaggccgaggcaggcggatcacgaggtcaggagatgtagaccatcctggctaacacggtgaaaccccgtctctactaaaaatacaaaaaattagccaggcgtggtggtgggtgcctgtagtcccagctactcgggaggctgagacaggagaatggcgtggaccccggaggtggagcttgcagcgagccgagatcacaccactgcactccagtctgggcaacagagcgatatccgtctcaaaaaaaaaaaaaaaaaaaaaaagactctcaaggatttaccatctaaaatcatcaagtgtatatcatgcagatgaacaacagaaaacactgggagtattcaagtaattaaaaataacctttcagcaccaacagcaatttctgtcctgatggcaatctactcagagctagaggactgcacttaggataccaaagggagcttaggatgcaagagcagcctgcactaatgtactctgcgttagtttacatccgggcactttgctttacgtttcaaaacagacgccgcccttagctcattaaaggggaaatggaggtataatgtgttttaaagggatgtttcttctctgaagccatatttcagggtagcatgaaaacagagctttggatatctggttccattctacatgacacctcatgcttgtttcaaatgacaccacaaccaaggggcaggaatgaaaggaatatgctaaaaaaaaaaaatgcaatcctttttagcaagaaagatcattaaggatttcctgataagtttcttctttgttcatgtgccctcctctgctccccctttgaactactgcccctgtcacccccccttccccgcctccccgccgtttctcagttctccaatagggatccagtctgccagaggtctatttttgctcattattgtcaacacaatttgccatcaagagttttcacttactcctgctatcctatcatcacattatgcatgtcagaaagcattacagatttttacttttcaagaacccgaactcagatttccaggcttctgtccttcctgtttcatgctgttgactctcccacatctttgtcttcctcctagacctttggtcactcttgagcccacattgccaatggtctatatcattttcacctggatgcctcccagacacctcagtcacatcaagttgaaatagaactaattagacagaagtagcatgtgtttcagtggaactgacatttttctagggtccctaaaacacaggtgttgtttcaaactcccccctccaacatcccttatattctctctgtcacccaggctccttatatcatctctcaacatgcttcctgggtgactactgactctccacttctggtgacaccccacagagcaagctttcaattcctgtcatttaaacactttagtgcactcatagaatctcatccttctgagatttctcttccaccagtctattttgcagaccaccaggctacgcccttccaaaagctcctttgagatggagtttcactcttgttgcccaggctggagtgcaatggcatgatctcggctcattgcaactgccacctctcaggttcaaatggttctcgtgcctcagcctccccagtagctggggttaaagttgcctgccaccatacccagctaaattttgtattaggggttttaccatgttggccaggctggtctcgaactcctgacctcaggtgatctgccacctaggcctcccaaagtgctgggattacaggtgtgagaaaccacgcctagtcccccaaagctccatttttatcctgtcttcattctctcttcaagaacttgcccacagctgccagagttccttctaagagtttaagactctctgttcatgtggtaagaatcttcttaaggagcttgctttaaaggtaaaaccagatttacacatcagaatggggagaggtgggtgggagctaagatggaatctgtaattttcacaagtgccctggatgattctactgcagatggtctcctattcaactaaatagcactcatctgacccactccttttagaccccacttctgttaagccagtgttatcgctattttccactttctttcaagttctaatcatccttaaaactccaacagcttcagctgaaatcagtaccacactctcatccttgtttcccacccaaaaactattgtactaattgaccagacatgtattgatgccctactatgagcctggctccatgataggcacttgggatagagaaatgtaccataatcctgaccttaataaatttagagtctaatggaagagataaaaaaaatcaatgattccaatgagtgtgataaggtaaggctagtcagaaaagtcaggtgcagaggaggggcaactaattaaaccaggggttgggggagacactgccaactctgtgttgagtgaagtttaagcagaggtttttctttaagtaaaaaatcagtttttttaaataaaaagttttttaaaaagtaagggggaatagaagaaggaggtttgtccctaaggggaacagtgtggaggaggaaaggaggtgaaagtgtatgctattctgtgtactggcaaattctttggtgggtgatatggtttggctctgtgtccccacccaaatctcaccttgaattgtaataatccccacatgtcatgggagggacccagtggaaagtaattgaatcatgagggcaggtttttcctgtgctgttctcatgatagtgaataagtctcaggagatctgatgattttataaagggcggttcccttgcacatgctctcttgcctgccaccatgtaagacatgcttttgctcctcctttgccttccaccatgattttgaggcctccccagctatgtggaactgtgagtccattaaacctctttcctttataaattacccagtctcagctatgtctttattaggagcatgagaatagactaatacagtaaattggtactggtagagtggggtgctgcagtatctgaaaatgtggaagcaactttggaactgggtaacaggcagaggttgaaacagtttggagggctcagaagacaggaagatgtggtaaagtttggaacttcctagagacttgttgaatggctttgaccaaaatgctgatagtgatatgggcaataaagtccaggctgaggtggtctcagatagagatgaggaacttgttgggaattggagcaaaggagactcttgctatgttttagcaaagagcctggcagcattttgcccctgccctagagatctgtggaaatttgaacttgagagagatgattcagggcacctggcagaagacacttctaagcagcaaagcattcaagatgtcacttgagtgctgttaaaagcattcagttttatgtattcacaaagatatggttcggaattggaacttatgctcaaaagggaagaagagcataaaagttcagaaaattggcagcctgatgatgtgatagaaaagaaaaacccattttctgaggagaaattcaagcctgctgcagacatttgcataagtaagatggagccaaatgttaatcaccaagacaaaggggaaaatgtctgcagggcatgtcaaggacctttgtggcaacccctcccatcacaggctcaggcctaggaggaaaaagtggttttgtgggcccagcccggggacccctgctctatgcagtctagggacttgttgccctgcatgccagttgctccagccatggctgaaaggggccaaggtacagctcaggccattggttcagagatgcaagcctcaagccttggtggcttacacgtagtgttgggcctgtgggttcacagaagtcaagaattgaggtggatgtacagaaatgcctggatgtccaggcagaagtttgctgcaggggtggggccctcatggagaatctctgctaggggagtatggaagggaaatgtggggctggagtccccacacagagtgcccactgggacactgcttggtggagctgtgagaagagggacaccatcctccagactccagaatggtggatccaccaacagtttgcaccatgtgcttggaaaagctgcagatacccaacaccagcccatcaaagcaaccacaaggggggctgtaccctgcaaagccacaggggcagagttgcccaaggctgtggtgggaacccacctcttgaatctctatgagacatggagtcaaaggagattattttggaactttaaggtttgactgctttattggatttcagactcgcataaggctgatagcccctttgttttggacaatttctcccattttgaacaggtatttttacccaatgtctacacccacattttatctgggaaatagctaacttgcttttgattttacaggcacataggtggaagggacttgccttgtttcagatgagactttgaactgtggacttttgagttaatgctgaaataagtctttgggggactgttgggaaggcatgatttgtcttgaaatgtgaggacatgagatttgggaggggccggggtgaaatgatatggtttgtctctgtgtccccagcttcatcaggatcctcccacacatccccattctaagttgcagggtcccattcttttccaatcaatgccctcattttaacagtagacacctggtgaggctgtgcatgcacctttcattgcaggtcagccgctcccatgataagagcttgtatctgattttccacaatttcagctctttctctacaagagataagactctcactctggacaatcttagaagatttgaggctcagtgtatgcttctggagctgggagttagaatccctaagttcatggttttcttctatcagtttgtccagtgaacttaggagcaaccaaccaacttcgttatattccttggtcctctatatatggtcaaaggtattatgtatagagtcattaaactccttgcctctcatgagtggtgaatcaggagtactgaatgcatttcttttgcataagtgtttgaacagttcgtgccaaggactatcagtgttctccacacaattagaagtagagtccttagcattttggggtctaatcatattaagcaaccaactccagaaacccaaaaaccaactaaagaaatctatccttctgtaatcccagcactttgggaggccaaggcaggcggatcacgaggtcaggagactgagaccacagtgaaaccccatctctgctaaaaatacaaaaaattagccaggcatggtggcgggcacctgtagtcccagctactcgggaggctgaggcaggagaatggcatgaatccaggaggcggagcttgcagtgagctgagattgcgccactgcactccagcccgggtgacagagtgagactccagctcaaaaacaaaaaaaaagaaaaaaaagaaatccatccttaaaattctgttcctctagaaccattcccagtaccaaaatctgattaaaaaaaaaaaacaggcagaggaaggtggaaggactagatctttctccagtgctggatgcttcctgccctggaacatcagactccaagttcttcagcttttggactcttggacttacaacagtaatttgccaggggctctttggcacttggccacagactgcaggctgcactatcagcttccctatttttgaggttttgggactcagactggcttcctgactcctcagcttgcagatgcctattgtgggacttggtatcttgtgattgtgtgagtcaactctcctaataaactccccttcatatattcatctatcctattagttctgtacctttagagaacactgactaatgccgtggggttaagccaatcgtctagtaggttcataaaatgctaaaagcagatataaacttaaagacgatccagctcatctcttcattttacagatgtaaaaacagaagtagaaatagaattggaaccccagtttcttgaaacccagtgtttgcataataccatgctaatttcattctaatttgtgttttatttaataatcaggaaacagttcaaatagaggcccagggctctgaaacattgcccaaggtctatggcttttgcaaagcaagtactgtttctgctactttacctagttgctcttggcctatgtttggggtgcatctaaagaactgtttgctgattattaaataaaacacaaattagaatgaaattagcatggtgttataaaaacacagggtttcaagaaactggggttccagttctatttctacctctaggatacagggtaagtcacttcctttctcagattctgtttctttgtatgtcaaagggctggattagataaacttgacttcccctcttggccctcatgtatcattctataaatatgttatcatttctaatagactgtttgatgtaatcttttgtctaatggcccctgcttttcacaataaatgaaacaaaggtcacaagacttttattcatttgcaaccctgattaactaacagttaatgtgtaactggagtgccacatagaaacagaaaggagaaggggaatggattgggttggagaaggtgaagtctgacctatcctctacaagaggtacagggtttatccaggaagacaggagtgccatgaggagtaaactccagcaggggctggagtcatggccacacacagggccttacaggatccaggctgcccagagcagagtttggatgggggctgggaggctgggggagctagctgaggagatggttgtcatgccaggccacgagtgtggatgagtgctggccaggggaggtataggagattgatggggctgtggcaagcagggcagatacccttccaggaacctgtttcattaaacacagaatacagtcctgagggctcggtctcaatacagtcctgagggctcggtctcaggaaaacatgttcttaagttttacatccttcttctgttttgattaggtgtttcctgattataaaataaattcctagaactgttaatggtaacaacacaaagcactatagacccatacgaggatcatattgaaaccaatgacatttaagaataaaagatctgatgaatatgaacacttccctggttcctaggaggataacagttgagttttgctcaggtaatctgccttctcttccttcttctctgctttrtatctcagtttctatattttgtcttacattaataggatttgttctcattaccctgatgattttatggtaaactacctcaaattctttttggaagaagatgggatataaattatttttaaagtttatctgaatagatgttcttcaatatcacacaataaataatgacaatgtaactttgtgcatatagcactttaagagattgatcataaacacaatcctattaatcttaattcaagttaataatgcttgcatttgtatggcattttaggtattataaagttttttttaattgtggtaagatatgcataaaatttactatttaaacctttttgaagtgtatagttcagtagtgttaagtacattcacaatgttgtgtaaccatcaccactagccatttccagactttttcatcatcccaaactgaatctctgtacctattaaacatgacctcattctccacctccccacagctcctgggaacctctattctactttctgtatgaattttcctattctaggtgtctcatataagtggaatcatacaatattcatcctttggtgtctggcttatttcacgtagcgtaatgctttcaaggttcattcatgttgtagcatttatcagaatttgattcatttttaaggctgaatatcttccattttatgtgtctaccacattttgcttatccattcttctgttgatgaacacctgggttgttttcaccttttggctattgtgaataatgctgctatgaacactgatgtgcaagtacctgtctgagtctctgctttcaattttgggcatatacctagaagtgggattgctggatcataggatcattctatttttaacttttttgaggaattgccataccacccgctacagcagccgcatcttttatattgccaacagtgcacaaaggctctgatttctccactttctggtcaacatttagattatcattctttttttaaaaaaacgtaatasctaacccaatgggcatgaagtaaggttgtttttgttttttgtttttaatgtgtgcgttatcttacttgatccattaaatccctaacaagaactcctctagggcagatgtcctgtcttattcatccttggccccagtgtcttgcaagcaagtgaattctcaataagtgttaattgaatggatggttgatagatttataggatgcatgccaattctgtggacgagagtaggtactaattgttattttcatttcacagaagagcaagtcagggctccgagcactaagtgacttggctgaggtcaaactgcctgcaagttttatctaatagtgacagaggaaccaatgtgtcgagcatgaatgtcagtccattgaaacagtgcccacttttctgactctgctccttaagagacagggcctgtacagcaaggacacagagaagcaggttacagaaaaagggctggctcatccgtgtatgcctggcatttgaggaatgtggctgaaatctcaacactctggttcagaagcacatctgcaatcaaatataacaagacatggtatgagagatgtctggcataccaaggagattcctagaatacagcggataggaaaacctcattacttcaattcccaagaaagagtactactggtagtaatcccaacaggagtatccaagtaactcggtaatcttcagtaaagaaaagaaattgtgaaacaattatagtcagtcctccatctctgtaggttccacatctgtggattcaactcacctcatattgaaaatattaataaataaataaataataacaatataacaattaaaataatgcaaattttaaaagcataacaactatttacatagtatttatattgtattaggtattataagtaatccagagattatttaaaacatatgggagagtgtgcataggttatatgcaaatactacaccattttacataagagacttgggcatctgtggactttgttatctgcaagggtcctggaaccaattccccatggatactgagggtcaactgtaccaagytgaaaacaaaacaaaaggaaatctattgcaaaaaggaaattcccaaagataaaaagcatgtctatgaaatagtcaaagagccgaaaatgttgggaagacccacactgctttccctgccctgtgcccctttttgcagatctatttgtgtctttatctaccagcagatattctattattctaatagattcctgtttttcccaagagggtttacttatttattaaatatgatgcaaacatctctctcagagatgttgctctctcttcctaggattccttagccctaaatctgcagagccaattaggtattaatattgggctttacaactttggaccatctgaccaccaagactgattcattaaatgtatatggtttcagatcacattaatttatcatagactttagaattggctttttagaagtactgctataaggaaaatctcagcatagcaagttttatctaatagtctacttgtattaaagagtactcaatgtaaaccctaggaagacttttaactgccttttggaaattggttgagtgggatttgaaccgtgtactctctgtaaagcaggaaattatcacttagtaattactaagtatttaaaaatgggaaatagaaaattacatttcagacctggtgcagtggctcatacctgtaatcctaacactttggcaggtggaggcaggtggatcgcttgagcccaggagttcaagaccagcctgggcaacatagtgagcaactccatttctactaaaaataaaaaaaattatttgggcgtgatggggtgcacctgtagtccccactactcaggaggctgagacaggaagattgcttgagcctagaaggtcaagcctgagtgagcagtgaccggccattgtattccagcctgagcaacacagcgagaccctgtctcaaaaaaaatgtatatttttaaaaaaagaaaattacattttagttgttccataaatatcagtacaaccaaacctaagtgcaaaattcccaacacaaatgatcgcctctgggccagccatgtagcccccatcttgctgtcaataatcatttccaggggctgtaattgtcttccttccccctgtaccaccccatcacaggacacaaattgttttgcttagtttagatagctgtgactaaactaagtgccaattgtcttttaaaatatgtgttaatcagcactcaagattgttcctacaaaaatgtcactccctcactcaatttgcatgtgccctcctgagtaggagagaaagagctgagttagaggcagccctctgggacctgcacagaaccctgtatgcttccgggagtgtggagtgtgtgtctgatctgctgctgggaaaaggagaagaaatgctgagacactcactgccaggggctggtatcaggccattttcacaggggctgttggagggttgacagcacagctctactggaccagggagggtcccagccagcagggcctgcccctccgaaactgtccctgtccctgtccctgagaggccccactgagtgtcagatggcacataagagatttccttatgcgttggtgtgaaggagatgatcagttccaggaggcccctcccccatgcagaagagaagaaaatggaagaaaccgggttctcagagtggcctgcgggtgagtgccgccttgtgctgtcagttcccttcaccttccagttctgggtgtacctagtgtggtttcatcaaactgctgaggccctggaattgagggaggatgctgaagggtgccaggccatagaagcagtagcaggagctgcagcaaagagctagtgtgtctccagcgtcagcttttgggccttggtggcatcaaaggagacgtacacagggccactgtactacggatgggattcttggaacaaaagtttgaataattgatgagtgtggaagtgctatgcaaaaattcaattcaacctgcagttaccaggcattcattctgtgacaggcattattgtgagggtggcaaggagacggggagtctctgagagactcagagaagagcagggcatggtgtccacccacaagagatctgaagactttcaaagtagttgggaaaacagacatacaacttttttcccctccccacagctagttgaggcaaaagacatataacttatacagtgacaaaatactactgactgttactgacacataacagcctgggctgggtcactggtgagaaaaaggaggattcttgcttttggggcattcaagtcctgtccattaaccatttcccaatcccatatttatacatcccattttagacatattaagctgaagattgatgtgacatacccaaagacacttaaggttgaagctgagaatctgggctagaaatataaatcaggatgggctgggagcggtggttcacgcctgtagtcccagcactttgggaggccgaggcgggcggatcacttgaagtcaggagttcaagacctgcctggccaacatggtgaaaccccatctccaccaaaaatataaaaaattagccaggtgtggtggtgcatgactgtaatcccagctactcgggaggctgaggcaggagagtcacttgaacccgggaggcagaggttacagtgaaaggagatcgtgccactgcactccagcctgggtgacagagcgatacttcgtttcaaaaaaagaaaaaaaaaaaaaagaaagaaatgtaagtcaagattaaagacaatgggtgagatcagcaaggagcatgtgtgcggagaagagaacaccaaggaaggctgcgtgtggtgggaggtggccggggggcagagaaagaggcggcggagccaaggagatagggcatcgtctgaatggtgatgctgtatcaacagatgtgaaattcccagaggtgtgaaacacagcagatcctttacagcacgatgacaggacacagcatgagcctacctctgccaggtgagaggagctttctgcaacctgtgatgggctcaaggatgctgaccattcatccgcccattgggatagcccaggctctgagctcagcactccacccgtcactgcgattgcactaatcctcacccacccttgcaggcaggtattactgtgggcacagagaagtttgctaacttgccagagatcctgtgtaggaagccaggtcaggaaacaaccagagtctctctaacagcccaggccttgaatgaacaccggcctgcttcagaatacatggcccgtgatgtgtttgaattcacagattcactggacaggttccctatagggcctgtgaggaaatctggtctaacagaatccagaaagacaaatttcgctaaacaggtcaagcctgatactgctgctacacttcagctgtgttaagccactcggttatcagacctgcttctcctttcatgattttagtaacacaggcctcttccttgggcccctgttgctcccaccatctcccaggttctcttgtactcagggtttagtctcccctccccactactaatgatgcatgtgccttactctctgatcatctctcctaaatctgctactcctctgctctttctgtgccttatctatccctgccgagtctaacatgcagattttggtcattccaaagtcatgtatagatctaatcacagggctctctgcttaccagctgctacttggataaggaaagcatgccaacacggtcctccttcttcatgctggccaagtcagcatcattattattacctaagtttatttctaacacatctcaatatcttcatgcactccctcttgataaaagtaactgagcatagcaccatcaataccatcaaatctgtcattctcttcccctctctctgggtgggacaggaagccaggctgctctaggaaatcttccctaacaagcaaaggggacttgcctgtcctctcgcatgtgtgatctgagcttgtgtggatcccagagtgggcattctggactacttgaccttgcctatctctccttcacaccctctcatctctccctcctaccaccaaaaaacttgcatcgtatttccaatctccagacatacttttgaaaccatttatctatctggtgtgtttatctgtatctagtatgatgtgaatatgtgatttgtatgtgtgtctaccactgttttggtcagtttgtatttctcgtgggtacagttctatgtgctcatgtatgtggaacatatgtatactgacacatggacctagctccaaatgatctgaaaggaatataattgtaattgaatatttgcacagatatacaacatacacatgtgatggctgggggaaatgcatgtgggatttcagtcagcattttattagagaaggtatgtcattagtgctgtattaacaatgaatcagcttatttgtgggtcactgtcaatgactcctttgcaaatcacacatgtaaatatttctgtctgtggtctgatgaacatgcagtgccacagtctgggagatgctgagccatgccctgtgtaggcagcatatgaaaagaactgcatgatttaaaagatgctgaccagcttaaggaaagcaatttaaaaacttcctaaaaatctagtttgaatgaacaacagttttcattttgtgtgtgtgttttttcttttagagatggggtcttgctatgttggccacgctggtctctaactcctgggatcaagcaatcctcctgcggctcagcctcccaaagtgatgggattacaggtgtgagccaccgtgtctgctggccccactgttttaaaccctgattcgacaatcatacatttaactcattacctgtcttgttccttttacgacaaactcaaagcttttattttactaaagtatttgggttaatcttttgcttttctgtcatgttctgaaatatgtacaataacagaatcgctcaaaatattatctccgttaaatgttttgtggctttagggagaggtctaacaacatgcgggaaacaagaaatcaagcgcatccaggattcatttataatctctctcgttgagtagaagtccgcatctctcgatattgtctggttacctgcatgaagtattctaaaggaggaaaatagctcaaagaggacattcatgtgcactctggcttccagttggccatttgagtaagtgatcgcaattaactgacgagcggcagggaaacacttcctggaattctcatctacagacaagaacaaactggggcggggcccatcaccttcacctacgcgccgggagggtggcggctggcgggcggggccgggctcgggccgtgacgccgagagtgcggggcgcgcggctgggagcctcgcgcccccgcccgggcccgcccccatcccgcccgcatacagcccgcatcccgccggggaagcgagcccagtccagcgctgcccgtccagtcctcgcccaagatttaaagcccgcaagttttgttcttgagaccagcgactttagctccgatgcgggaaggaaagccgacctccgatttggacatttaaagagctgggcttgaacttcgtgagtttcgctctaaactgcccttgaaatgaagctggacttggaggtaaagtcactgggaagctggcctggggcggggtttccccctcttctcgtattttagaaacggacagcggcagtgcagccctagtttgctgtaagtttccttactttgttactgaggcccccagagctccacgcataagtggtgtgaccagaaaccttttaacaagacccgcctgagcctgcgttagagctcccgctcggaaagtaaaagaccatcctaatccgcggcgctgcggaaccggtgtcccgtgtgggaggaaccgcggcgttccctgggcgtagggcccgcgaggccagcacagtccgcctcttggcggagcgccctgggccggtggttccgcgcggagttagtctgtggtcagttacgtggtgaaaacacggctgtgccgcggccgcatctttccgcggccgaggcctctctgggtgggagtgttggcttcctttccggatcgctaaatggggaaagttctggccgctcggcgggatacgtctccaggccacggatggttcgttctccgtgccgcggccccgagctgggctccctgggtctccagcgcgggctcccggcattgggggctgcgggccggcccctccgccccgcccccgccccgccgcgcctcctcggccgagcggctcgcggtctccggcgcgggaggctccgagtctgcccactccgggccgagcgaggtctctggaggagaagagtggcgaggaggtgagggcacgccggccctcgcccggcgggtggcgccaggacttcaggtgggaacgcgcgcttgggccgggggcgcgtggctggcgtggacaccggatcggggcccgccgccctggcccggaccgcgcacggcccagcgccgggaagtcgggaagccggggaggcctctcccaccgcgggccccggcagcccgccctctgaaagcgcggcggagaaggaggctcgtcccctccccggaacgcctttgttccctccggcctgcccgcgcgggtggccagcggctgggacccaggccgggccgccgcccaggtgcggcaggtaggctcgggggccgggcagctccggttggggcggcttcccggggcctgcgggtccccgtccctgaggagctccggctcctcggtggcgggacaggcccgtgcgcgggagccgcgaggcgaacgccgcgcccaccaattcggttgccggccgggggccccaggcttgcggccacccgcctccggctggagggctgaattcgagtcgaaagcccgtgtcgggctggaaagaagaaaccgccaacctgagaacgctttcggcgagttactggcgggggaaatggggacagggaagtgggcaggcggggagactgcagccgcagatctccctggcggggaggtcgtggccactctttcctttgactctgcctcatttcattttgaatcctgatgtgacagaggcaattgcttgcttggataccatataggtaaaagtaacagttttcaactcgactcttgactacaccctgtacattcttggccggtgttggttttcttaggttatggatcatgttaaaggtacaccgatgtggtaaccgcacagtggccgatggtggcctgaggctcatatttatggtaattatctgatgaaagtacatccatcagaattggatttgtgcgtctgtgcctttattttgggaaaccttgcctgttccctgtgggggatgggagaggaatgtgaaaagccgaagttgacccagaaaaggatgattgaaggtagttgtattaagtggtggcaccgaaatgattccaccctgaactttctgaaagggtgattagtgatcagcagcagacgttataactttctcaaaataaatttatggtagattttctatctggtcacaagtgaggaggtgaaagctgctttttggggccaactctttgcttttaaagcaagctaaacgcaataccagaaaggtttccattctgtacttaacctgcttgtccttctcactcttccttatcctcccgcacacgctctgagcattgactgagcactctgaggaggaggctgactcaggctgacccttcccggccctgcaaggctctaaggtagaaccagtgttatcacaggaaaggcccaagccaaagctcagagcagctgttcctgagaaagtgggagatgaggatgagtaggaggtagagatgcttataactgctctgcccagagaaaactcagaaggtgcagaagagttttcaaaataaagtgcaggccccatcaagtaaaaaatgaaacattgtatttcattaaaatggtgatcagttttctcttttcataaaagacattcaaatggtgagattgtaaaaaaaaaaaaaaagaaaaagaaaactttctaattctcaaaaatagaacagcttgaataataactcatgggttttgaaatgggtcatcttttaaaatgggtcactttggctagaatcattgcacttggtgactttcacattgtaaagggtccagttctttctgaggcccaattgcactagaatctgcatttcagaacacaggaggtatcaggaagaaactgggagaaattcagagggaaattgtctcttctcagagtaacaaaatgtcccctattcagggggaattttatatatgctgaagtaaatagatctcagctttgaatttttataatacattatgccttttctgaaatatttccatagccattattttaatgcattgttagaataaccttgtaaggagaggaattcattgtatctgtgttccagaaactgaggcacagtaaattacaagagctctgccctttgatatcgaatctctgtgagcgaagatgtaaccagaacagtgtaaatctagaattactttccttacggtatgcaatatctaattattatgtattagtttacatatatataggggatgtaagaatctttatatttaaaggataagaaacttgatcaggtgagatacaagatttgaataaaaaagcgattttttagaaacattttaattctacaaattcagttgctggctttgatatgtaacttattcagatttctcttccatagaaggctttaacactagaagccagttctctacaaaagagaatgttctaccaagtgtgtaggcaacaaagcccaaaagttcaagttcaaaatactgttctacagcctaagatccacctaatatttcagatttgactcttttgctcctttcctataacttcctataactttttcatagtgctctaagcaaagtaagttcaaggatggactcacctcaagtttccttctcttactttagaggaccatataatcttatacctgtgtcatcctaattgaaatgtattttaagtgcttgtgggaacaagaataacggaagaccgttattccattaatggaataatggggaacatgagaaggaccatattcagtcaattagggatgatacttctgcctcaacagtatttgcgatgtgttaaatggcccttagccatcagatcagtatttttaaaaactcctacctaattaatgttttttgagaagaagcatattatgagtatagctcagtattctaaaaagaaaaaaacctgaaaaaaaaatcagccatccattaactaacccatcctggtaaagctacacaaacagattctagagaaaggaaatttgcacagatggaacatctaatctggactgacattgtcaacaagttatctcaaatgatcctgagaaaacactggtacatggtttagagacaagtcgagctcatgtgactagtaaatggagaggcacagtatacatctacttttgttggcatttaaacactctcggcttttttactctctttcaccaccatcaggtccagattccagcttgcactggaatatttattgaccctttgatagaagagctacacctgaaacatctctgaagtctttttagcatcctatcctgcccatggcctactacacagtatgtgataagttaatgtttgttgaattagttaaatatacgtattaacctactttcagggctgcccgctgtaatgcctagaatagggcaggcacttaataaatggtagttaacttagactaaatgttgatccaccaagacagaaacatgttacatatacctcttgttttacaccatcttctactttttgtacctctgtgttataaaccctctatcaagtgttgcttctaaagacaatgtccagaatgggacgtaggaactgccactgggcaacaggcagacactgggttatatcgtcatcctgtttctgaatcctggtttttcttattattattcaaccacagctaaactttctgatctccttttttccgttttttccctttaccctaacttctagccaaactaagtgatttgacatttccccactcaagccacaactttttctcttatgtgcttaaaaaaaattcctgcgtagtctctcttggaatgtcttattccttgctccctccacttcaccctttttttttttttttttttttttttttgagacggagtctcgctctgttgcccaggctggagtgcaatggtgccatctcggctcaccgcaacctccgcctcctgggttcaagcgattctcctgcttcagcctcccgagtagctgggattacaggcatgctccaccacacccagctaatttttgtgtttttagtagagatggggtttcaccatgttagccaggatggtcttgatctcccgacctcctgatccgcccgcctcggcctcccaaagtgctgggattacaggcatgagccaccgcacccggcccacttcaccctttaagccagttgaaatgctccttgccctactcctcccttctccttttctttctcccatccaagtactaaccaggcccaaccctgcttagcttctgagatcagccaggatcaggtgcattcagcggggtatggctgtagacttctctttctgagctgtgtcttcctcctttcctccccaaacccacatttgaattctgttctagctcctgctcatttgccacctctcccacaaagccttccccagtttccatcttctccctgcttccctcatctcagaggaaagctcttcattcacagagcacctggtttggggtccatagtttgttgtccctcagtatccttggggaattagtcccaggacaccacccccccgcccctgatatccatgcatgctcaagtcccttatataaaatggtacagcatttatattataacctatgcaatattcccatgtactctttttttttatttttatgtgtttagagatgggtctcactctgttgtccaggctagagtgcagaggcatgtagccttgagctcctgagtcaaatgatcctcctgcctccgcctcccaagtagttgggattacaagtatgagctaccacacctggcccatgtactttaaatcatctctagattacttataataactaatgcaatataaatgctatgtaaacaattgttacacggtattgtttagtgcataatgacaagaaaaaaatgtgcatgttcagtacagacacgaccataaatttttttttcaaatgtttttgatctgtagttggttgaatctgaggatgcggaacccatgaacgtacacgaccaactgcatatgggtttctaataacagatttgtggatgaagcccacccaacataatgcaacatttcaaatgtatcttagtcagtttttctggggcaaagccagcctaacaaatctcaagaatgtttgaaaaattctagcttgagtgaacgttgttcttctagtgactcaaataaagcagtcattgtgtctgaggagtcctagtggggtggcaggggtggggctaggagtagtggctgtttgtagcatgttttatatcacattaaagggctgggtatgcattttaggcaataattcttactcttattgataggagaacttgtattttttattcatctaccttatgatggataactggtttacatctcttcatcaggtttacttcttacgtgtcattttatgagctgactttgaatatatcttcagattttccctgcctttacacatgaaaacggttttgatacagctttttcattagagaatggcagcttttccaggccagggtgtctgtgtctgactcctcgcctttcattttacccaatacttgcttaaaacatcctccctgagtccggtacttttttgcctcctctccttccttccaccccacctctttttgtcaccctcctgcgactttggatctccatccttggagtccctgaatcttttttgtggttgaaagactacacccaaaggacacagactgatgaggtcacttctgcccactgtctagtactagtgagaccaagaagaagaaggagggaaaactgaaatggaaggtttgaaaaagagtgtgttagtgaaggaagtaagaacagtgctaacagaatgcagcaaaacagaaatgacttcacacttctggcagtgaagcaaattctcatccatcttgggttgcttctcttgaccctgcccacctcctcccctgccctcccctcccgatgagaactgcaggaacacctgtgcccatgccaaggaaaaagggcaggtgaggaacacagggatggggctgggactcacaagctgctctaagctgccacagggtctggaatttgttgacattacatggttaactcggtatctcgatgcctcagtatcttctgcaaaatgtggagggagatcctttcttcctcaaaggttgttgtaaggcttaaagagctaccacattcgtaaggcatttggaacagggcctggtatatagtagacatacccatggtagtaactgttgctagactgggtaaaaggagaagtttcttaagtaggcaagaaaaaaactgacaacagttatatttaaaagagcaagcaaccgtgtcagcccaaacaagtgtgttgcaagaatgagcacagagggccaagctcagaaaagcgagtgtttgacctctctgagacctcctttatatgtcagcttttttatttgtttcaaattttgcataatcaaactggttactgggaattattgagttcatataacttccctaaaatctaagaataatctataatcccatgccaccagtcccgttaataaagacttagggtctgtctttacccatttgttcatttgaaattccccgtctttacttcccccataaccaggaaacagatctacacccatgtttaacttataaagagatataagtgagtttacataggtggagatcttgtacctggattcacattgtaggttttattacccagctttcccctctatcccaaagccatgattgccatagtggaatttaaagtctttggactaacactgaatcaagaactaactctacaaatgtattctctgcaggaggataagaattccaaatggcttctaattgttgcccatggcttgaaataaagttccatatagctaaagccccagcacaaaaacacttgaagacagccattaggtagaatttcttttcttttttagaggtcttatattggaaatgtaacagttgcaaagatatctaatgtttcaccttaaaatgtgagttataaattcacatttcacattgcctccttcctccttgagcaaacagactaggcaattagtaccaaatagtaactagttatatttcttaacaccgattattgtaagtaatttattaagaaaaaatctaggaagatcaaattatagcttttccagtaaatctggggctttttattcctgatctctctcaacaaggcagtttggcttctgaagattggtcctagctatacttaacaggaaacagatgacgagaagaagccaggaaaaaatacttggaatatagaacaaaatgcagcataaagatggtacagaagattttatctttctttcctttagttatgtgtggacaggaactaaaaactctcccacgtgggactttattaagtaataaaagtttaaatgtaaaagatataaaggaatgaggatataaaggaaatttttttaaaaactgtgactcaccggaggagccttatatagtctactttctaagcatatcacttttaaagggatcattatattttcatgatgcatggggatattccaagtattaaattgattttttaaatgaagaaaattatcagttcagtcttgttttctatgtaggtttcactacagtattttatttcccccgcaaggaaaaaaccagagctagaatggaatgattaaatttcttagttttcttctaatctgttagtctttccattaaattatactgccttcttatatagaatttctgggtttgttcttgttttggcaataattgaagtaagagaggatgattttatggtagaggtttgattccattctagattggtgtttttcagctatgtagaacacaatccattcatggttcagacaaccgttttaggaggttttcatgtccagcattaaaaaaaaaaaaaaaagaatagaattgggcattcatatatatatatatatatgaatatttattaataaaagtttaaaagatataaaggaatgaggaatgcatatatattatatatatatataccaaattaaagtatcataatactaatacaaggattagtatcgttttatgatactttattatatacactaagggttagtcttgttttatgatactttattatatacactaagggttagtcttgttttatgatactttattatatacactaagggttagtcttgttttatgatactttaggttggtatacatacatatactaagaatgcagacatgtccatggaagttggcaatattcagcccccagcaggccccagtcagcaggacagggattaggggagtcaggtgaggcagggctgtataagtgcagggttggagcctgtcttggtttaccgttttgacattttgttcactgtggatttttttcagtaactttgattttttaaaaatattgcatcaaaatattatactgactacggagtttttggtacccccttaaattgtgcacctaaaataagtggctcccttgtctccccctagtcccggcccgatcagtatgttttatagagggaatggtgagtgttggggaactagggagcccatgaggaggcggcccagaaggggaggaggagctgatggtgtgtctgatgtctcctcctttctctaggaagcagacaggaggtgaagtcttcagggtgggggggacagagcctgggagggggagatttagcatggccgctgaagagactgggaaagggagaaggttcgggacaagttcaggaacagtcacgtagcactgaaaactgctcaaagctagatagtgccatttgtggtgacgctgcctgcctggtaagtcccatttccccagcagccctggaggtgtcataggaacaggaagatgggagggcgctggggtctggggttgttggggtgggagggcaaaggtgtccaatgattctgcatctgaaacgggggaggtgtgtgaggaagaaaggtcaggagactgctgggcagggtggggtggcagaaggttctcgatgaatatttagaagttgcagctgaggagtgagtgtactatagagtgagtatcctggaatttgaggtctcagaagaaatgcagttctcagtgaagccatgtaaacaacttcctggctgataaggtgtcttgagtgttgggagatcacatgatcaaaatggtatacacaccataaggtgtccttagacttgagtgtggtgtaagtctactgaataaaatgcagaaatacagttcactgttgcttttcttcctggaaattgccctccccaacctcaaaccagcccccaagtggggcagggtccatctttagaccctcctgtagcacccagcacaaccctaactatatggtcaatgtctctcttccccacttagagctccaagaccagggccttgtttgtcctgcttacccactctacccatttgttaaccccatcttaattttctgatgaatgagcccttttttcattgactcctcctttctctccttctccggagtttatttttcctccaggcccattgctgtctctccacctagagagcttatccacattcatgaaattagtcatcttcatgctcagagtacccacctggatctctccagtcctgtctaactcgtcttagattattaccccctggacatctgtaccttcttgtctctccatcgcctcgaactcaatgcgagtctaaaatcaaatactccttcactcctaactcaggaaaccagttttccctcccttgttggtaatgcttgaaattagctcttacttctcttcttcctcctttactcgccatcccaggtcattcatcaggtcctgctaggtctttgattttcttgaaagggccttctttatgccttccttccgtccccatttccaccaccctcgactaggccttcctcacctcatccggcatcagtgtgagcccttgagaaaccagcgtcctgatcttcaggcattctgcatttcaccccataccaccattagccagtttaatccctcttgctcaaacttaataaatactgagcaagggagttactataacaaacaaaaccagaaatttctatgacttcctgctgctttctgtacccccctaagactcctctacctgattttcaaggttctccataagcaggttcccacctctctgctttcaaatcttctatctgccatacacagttcttgtctaaaacaaagattgttcctactcctgcagccacctaaatcctagtcgtcctcaggccctcctaaaatcctgactaatccaacccccaccgatttctctactagtcccagagacttccctcttgtgcctggggttggtgtcatgcagacacactgtaccttaccaggatttgcccattgttgtctgtgggaactatgctttcatgtgggaactacgagtcttgtcacaaatagagtgtaaatgctttgagattggagactgtaaaatacttcttcatgatctcccactgtgtctggtataagaacccgcagagtaaatactcatatacttgtaggattaattgaaacaactggagactgaggttgtttaacttgtacttagaagtgtggacctccccatcttgattaaaacttcaaaacggatagcaggtttcacatattatctcagcatcacaaaagtagactcaacaaagaaatgagttgacagcaggggtaaggtttacaacaatcagggattattgcagggaaacttgtagcctttgggtgttctaactagttttttcccaaaaagtttaaagagctctcttgtgaaagaatattcttatgagtaactgaggggcacggctcctagactgaaaagtatttgggatctgtcacctcttttctgatgttcctacttctattctttattcttctgactacctctattagaaaagataatactaagaatacctgtcccttcttctcagtccaaatagaagcaaacccaggttgtatctgagatatctgcatattttcttctaagcaattctttgtcctcttctctcccatgctttttctatttcctattttcagcaggctctgaagtcatttataatttttactgcccctcggtgacattacatggatattcatccctgatttgcagattaaagcaccgaatcagagtgaggtgagggttgcccaaggttacacaataaatctggccccaaacaggggtaacccttgagtttctagtctgttgattggccactgacccgtgctgcaggcacacaaaggaagctgcacccacagcagtctgttgtggatggttgctgagctgcgcattcggcattgggcttgctttgtttcctgccaggcccagcattttcttctaccagatcggcaggcttgtgggcttcttcctaggtccctcccctgcactctgaataggaaagctggaagctgtgctttagagaagctttaagacgccgaaagaaaccagaagagtgagcgccagttgtatgtgcgtggtctccatccgcaaagccggagctgggcgcaacagtgttgacttgtaattgatcaatttagatcgggcgcaggccgggggagggcagtgcttttgatttaggctgggaaaggcctcctagtgactatgttcaatttggaggaattcagatgttcttttgttatacaagtgaagctgtgtaatacaaatgaggagttttacttttcctaaatcttccccttatcattcaagtattgaggagttttacctttcctaaatcttccccttatcattccagtattatcagtgagatctggttgtgatttatgtaaatggtggctaaaaaattcaaactactgagggggagaattctcattttacagcttcacatgctgtgctgaactaaataagtagcgtgggatgttggctttgtgacaggtcttttgtcatttttcagaaagcattttgacttgttgatgtcaatttggaacagctgaaaaaatacaggaaaataagataaatacgtacatgttgagggtggggacaaaatgaaggttctgaaccagctgccggcttacagtagccatataagcaacagcagcaatgcaccaacctggtgagtaataggcctgattcactggagagatactagcacctttaatgagtcagatagatgcacaatgggtgtgggagcagttggacttgtgggcacaaagtctagcaagaagctcagacttgcaaacaactgtaggacgtgcaaagcaagctggcattggagcttgccgggcacagctgctcaggaataggcagctggttttccctttgatccctgagattccaaaggttactttcctctttgttcccttcccagggtcaattagagtagaaactgcagatgcttttcagttgagaattttcctagaattctcaaaaatgtgtatgctggcttaaaatctgccatcaagaattctgttaccttgctttaagcctccagttccttccagatgtatggtggaggaggccagagggcccttgttttggggcttcagaggatggttgttatctggatgagcactgtggaaagactgagagagcaactgagagaaagtgggcccctgaatgaaagtgatttcgcaaattttaggcagatgccaccatcagaaactgatattttctgacgtctttctcaccttcctctagagcattcagtccagaaatgaccagcctgtccaaagggggaaattactgatattgatctgttccttagagcagtgtttcagtcttttttttttttttgagatggaatctcattctgtcacccaggctggagtgcagtggcacgatctcggctcattgcaacctccaccttcctgattcaagtgattctcctgcctcagcctcccaagaagctggaattacaggtgtgcaccaccacacccggctaatttttgaattttttatagagatggggtttcaccatgttgccaggctggtctcaaactcctgacctcaagtgatcctcctgcctcggcctcccaaagcgctaggattacaggcgtgagccaccatggccggccttcagcctttgtgatattaaagcacagcaacacatttcccattacacccctgaacacacacacacagaaaacccaaaagtttcacaaaatgattcttgctcttactactctcagtacactctgtatttaaaaaaaaaaatgctggttgtggcttcctaagtggtgcgtgcagttttcaaatcaatgcccttggcgataaagtgtgccctatactgattatctctggacaaagtctgaatggggcttggctctaatctctagtcctcattggacattttacatacctggcctttgcctccaccctgatgtggagtgatcatgggggtgggaaatatagctggatccgaaagctctgaagtggggatggaggtgtcacagctgaggctaggcccattctgcagggcactcagtgtgtacagttggttttctatcaggggtcaaccggcggggggacttgagaacagatctctgggcacaaagcagggcctttgccctggggcttgctatgtggctcagcctacacggctctctccccgtcagtcctgtccaaagcccaggaaactaatgtaccacccccgaggaagagagcctacctttccatccaaggaagtgttttacctgtggtaagcacgggggacagaattcttgaggaaggagggtgctgcgtcccagtggtggaggaaaagagaggacctggtgtaagcagccatggcatggacctcatccgaggtggcacctggctagggtcctgacctccaatccttccccagtaaccatcactttgagtaaacagtggctccacccccggcatggttctttgcaccaacatttggggaatgcctaccaggggtcacacactgagctggatgctgagtgtagggtgtccacaacatcgtgcctaaaaagtctctgtatggggtataagaaggtgctggggcaatacagatgagatgagaagcatctttcagggaatgggttgatcccaattcaggcttcccagagaaggatgtctgtagacttcatattagcaagggaggaaggtagccaggccacaggactgctggtgtaaagaccagggcatatgaaatggcaagtgtgactgtgctttcagccaataatttggtattgtcaaatgatgggaccaaacagctggagaggcagatcctaaagggtcctgtgggccaggctggacttcatcttgtcactaactaatggagaggctctgaaggagttaaaagagctcagtttgtctcgtggttaaatccaagttttacaaaggtcacgctgactgtaaagtggaaggtgggctggccaggggatcatctagtctgggtgagaagtgatgataacatgaaggggtgaagagagatttagaagaagtgattcacaggattaaacatttaaataatggaagtggagaaaatggggggggcggttccagatttcaggcatagatgaaagaagtgcagttaggcacatgtaaagagaaacaggaacagcaggttttaggggagaagataacagaatgggtgagaaatgacacttgagtaccctagtgtgctaggtaatcatctgtctacttcccttcatttgtcatgtatattcccatttaatttgcataaagacttcgagttaaacggtcttaccccaatttgtcaaatttctgcgcatgatatggtacaagaaaccgtaagtggctaaggcggcattggtgttcaaattgcctgactacaaaggcagtgcttgttggctacattctgttgcttcccagtttagaacatgttacattgaggcgcctgctgcatttccaaataaaaaagtacagaaagaaggtggctgtataaatctggggctcacaaagtaattttgattactgagagtttgctttcaaggagcaaactgtgactccttgattatgaaccttaatttaaaaaaaaagaaaaaagaagtcttactcttattcctgccttgtctggggcaagccttaatggatttttactgctgtgaattttcttttcattgaagattttgccttgatctatgtatctgctttcatcctgaccatattcaagtcagtatattcatgaatgtacctgtttgtgaaatttgaacttaagtatacacgattatagccgtttgggaagcttttttttttttttttttaagagtaggagtagaaaaaggtctctgtactctgaatgggaagacagtgtaaagcaattttttcccttttcctgtcctcctttaaaaaaaataaacagccgtatgcctctgctaagtactaactacctcatcaccttttgtgcagacagggcaggttacatttggttttaaggaattaggaatatgtttctttccagcaccttagtaacccacgcgattgtgattcttttctcttcttgactgtgataggtggcatggaatattcacatgggagagccgcatgaggccgcccaccacgcttcctgaaggatgcccgtgtggaagaattttgacgtgccagtgtcctcgttctacagggtgttccattcttccgcaatctcagaaaaatgggactaaaagaaactattttgtaaaataagaagacttccatttttaatgaccaacatgtattaagatggacacctactctacgaaacacgaagttctatggtctcgaagaagcccgtgcctgtttaaaactgatcctaactaaaaacagacttgagtggatatgagaatgttggttagtggcagaagagtcaaaaaatggcagttaattattcagttatttgctacttgttttttagcgagcctcatgtttttttgggaaccaatcgataatcacattgtgagccatatgaagtcatattcttacagatacctcataaatagctatgactttgtgaatgataccctgtctcttaagcacacctcagcggggcctcgctaccaatacttgattaaccacaaggaaaagtgtcaagctcaagacgtcctccttttactgtttgtaaaaactgctcctgaaaactatgatcgacgttccggaattagaaggacgtggggcaatgaaaattatgttcggtctcagctgaatgccaacatcaaaactctgtttgccttaggaactcctaatccactggagggagaagaactacaaagaaaactggcttgggaagatcaaaggtacaatgatataattcagcaagactttgttgattctttctacaatcttactctgaaattacttatgcagttcagttgggcaaatacctattgtccacatgccaaatttcttatgactgctgatgatgacatatttattcacatgccaaatctgattgagtaccttcaaagtttagaacaaattggtgttcaagacttttggattggtcgtgttcatcgtggtgcccctcccattagagataaaagcagcaaatactacgtgtcctatgaaatgtaccagtggccagcttaccctgactacacagccggagctgcctatgtaatctccggtgatgtagctgccaaagtctatgaggcatcacagacactaaattcaagtctttacatagacgatgtgttcatgggcctctgtgccaataaaatagggatagtaccgcaggaccatgtgtttttttctggagagggtaaaactccttatcatccctgcatctatgaaaaaatgatgacatctcatggacacttagaagatctccaggacctttggaagaatgctacagatcctaaagtaaaaaccatttccaaaggtttttttggtcaaatatactgcagattaatgaagataattctcctttgtaaaattagctatgtggacacatacccttgtagggctgcgtttatctaatagtacttgaatgttgtatgttttcactgtcactgagtcaaacctggatgaaaaaaacctttaaatgttcgtctataccctaagtaaaatgaggacgaaagacaaatattttgaaagcctagtccatcagaatgtttctttgattctagaagctgtttaatatcacttatctacttcattgcctaagttcatttcaaagaatttgtatttagaaaaggtttatattattagtgaaaacaaaactaaagggaagttcaagttctcatgtaatgccacatatatacttgaggtgtagagatgttattaagaagttttgatgttagaataattgcttttggaaaataccaaatgaacgtacagtacaacatttcaaggaaatgaatatattgttagaccaggtaagcaagtttatttttgttaaagagcacttggtggaggtagtaggggcagggaaaggtcagcataggagagaaagttcatgaatctggtaaaacagtctcttgttcttaagaggagatgtagaaaaatgtgtacaatgttattataaacagacaaatcacgtcttaccacatccatgtagctactggtgttagagtcattaaaatacctttttttgcatcttttttcaaagtttaatgtgaacttttagaaaagtgattaatgttgccctaatactttatatgtttttaatggatttttttttaagtattagaaaatgacacataacacgggcagctggttgctcatagggtccttctctagggagaaaccattgttaattcaaataagctgattttaatgacgttttcaactggtttttaaatattcaatattggtctgtgtttaagtttgttatttgaatgtaatttacatagaggaatataataatggagagacttcaaatggaaagacagaacattacaagcctaatgtctccataattttataaaatgaaatcttagtgtctaaatccttgtactgattactaaaattaacccactcctccccaacaaggtcttataaaccacagcactttgttccaagttcagagttttaaattgagagcattaaacatcaaagttataatatctaaaacaatttatttttcatcaataactgtcagaggtgatctttattttctaaatatttcaaacttgaaaacagagtaaaaaagtgatagaaaagttgccagtttggggttaaagcatttttaaagctgcatgttccttgtaatcaaagagatgtgtctgagatctaatagagtaagttacatttattttacaaagcaggataaaaatgtggctataatacacactacctcccttcactacagaaagaactaggtggtgtctactgctagggagattatatgaaggccaaaataatgacttcagcaagagtgactgaactcactctaaggcctttgactgcagaggcacctgttagggaaaatcagatgtctcatataataaggtgatgtcggaaacacgcaaaacaaaacgaaaaaagatttctcagtatacacaactgaatgatgatacttacaatttttagcaggtagctttttaatgtttacagaaattttaatttttttctattttgaaatttgaggcttgtttacattgcttagataatttagaatttttaactaatgtcaaaactacagtgtcaaacattctaggttgtagttactttcagagtagatacagggttttagatcattacagtttaagttttctgaccaattaaaaaaacatagagaacaaaagcatatttgaccaagcaacaagcttataattaatttttattagttgattgattaatgatgtattgccttttgcccatatataccctgtgtatctatacttggaagtgtttaaggttgccattggttgaaaacataagtgtctctggccatcaaagtgatcttgtttacagcagtgcttttgtgaaacaattatttatttgctgaaagagctcttctgaactgtgtccttttaatttttgcttagaatagaatggaacaagtttaaatttcaaggaaatatgaaggcacttcctttttttctaagaaggaagttgctagatgattccttcatcacacttacttaaagtactgagaagagtatctgtaaataaaagggttccaaccttttaaaaaagaaggaaaaaactttttggtgctccagtgtagggctatctttttaaaaaatgtcaacaaagggaaaataaactatcagcttggatggtcacttgaatagaagatggttatacacagtgttattgttaaaatttttttaccttttggttggtttgcatcttttttccatattgttaattttataccaaaatgttaaatatttgtattacttgaattttgctcttgtatggcaaaataattagtgagtttaaaaaaaatctatagtttccaataaacaactgaaaaattatcatgagatgtgtatttaaactttttcatgaacattgcttatataatcattccttctgtcttaatgtactacatggtcttagccctgttcctataggattatcatgttctctgcattatagagccacctaagatgtactttttgttaaatgactcatgctggaatatctggatggggagatgttctttcctaatgtagtcatgtgccacaaaatgacgtttcggttaacgatggatcacatatatgatgatagtcccatgaaattgtaatggaactgccctatacaggtgtaccattttttatcttttatttcagatttttactgtaccttttgtatatttagatgtgtttagatacacaaatactttccattgtcttacaattgcctgcagtattcagtacagcaacatgctgtacaggtttctagcccaggagcaataggctctaccatatatctaggtgtctagtaggctattccatctaggttctcgtatgtataacctgggatgtttgcacatcgatgtggtcacctaaagatgcatttattggccaggcgccatggctcacgcctgtaatcccagcactttgggaggccgaggcaagtggaccacctgaggttaggagtttgagaccagcctggccaacatggtgaaaccccatctctactaaaaatacaaaaatcagccaggtgtggtggcacacaccagtaatcccaactactcgggaggctgaggcaggagaattgcttgaacctgggaaagggaggttgcaatgacctgagattgtgccactgctctccaacctggacgacagagcgagactgtctcaaaaaaaaaaaaaatgcatttctcagaacttatcctcattgttaagcaatgcatgactataatctgttgagagagggatgaaatcacctgtagttatagcgctttaagataccatttgaaaaggttacgttttccttttctttgacacggttagctgtctgaaatacagtcaattttaaccctaatctcttaatatcaggaatgcccttacacctactttggagtgtctggtgcttcgatatagttgcatgtaatgtgctctcatctgtttttacctgattcctgctcagttcttcacatggcatattgtgtaactcaatctatatttaaaacttgtaagcatccgaattatttgtttatggtagaactttttacttgcaagtcgtggtaggagtgtttgtagttggtactaaaatgtgatgacttggaagaattaattaatgacctatatttggggactttaattggatgctatagctgcaatgagaatagaaccagagaactcttgatatgcaaggttattcattctgtgataataatgagaggaatattcgatgtctctttgagtcagttttccttccgatcacttccgcattctgcagtgacacaatccttaatcatagctttcattacaatattcctttactccagacctcaaagactcctcactgcctccagcatcaaatctaaactcttctacctggttttcaacgccctacgtaaacttttcctccttcattccctatagcttggtttttttttttttatcactgccactattatctatcacaaatgtcaatcatgaccataccttgcttaagttggtttcccttgccaagagcactgtttttcctatacctgttgaaattttggaaatccaattctacctcctctcttcccttaagcatctcttccttcccttctccccaaatttatatttagtcacatatattatcgtactacctactaatcattccatgtgtttttttacgagctgtaagttctgaaggcaaccatgccttgtacagtgtccacttagggttctgaataattaatcatctccccaaaatctgaaagccttctatataccaagcaaatttgtttagttatgcagcaaaactcaaatctataaaatcaaaaaggaataaggaaatacagattaaacagttgcagcaaagactggtgatcttaaggtatttagtcaaagctggtggtagaacaaaaacagtagtcttacagattctacctcttgattaactcagtggctaattttgccttttctcaaagttcttttgcaagaacataaagatatttttgtttctttagttgagtgctgtaactttattcctttgtgtttctcataagtatgatttggcagtctgccatacgttttttgttttttttcttcctctttgagacagggttttgctctgtcacccaggctgcagtgcagctgtgtgatcacagcttagctcactgcagacttagcttcctgggctcaagcaatcttctcacctcagtctcctgagtaactgagactacaggtgcaccccaccacatcccgctaaatgttttaatttcttgtggagatggtgtcttcactatgttgctcaggctggtcttcaactcctgggctcgagcaatcctcctacctcagcctcccaaagtgttggaaagtgttgggattacagacctgagccaccacacctggcctgttataagttaatacaataattcatcaactaacttaaagaacactaagactcttacaaaagtaggtatgagttttagtaaaagtctcaaaagataaactgtcacttaaggaaaactagagaacatatcattgccaaatggtgtttttcagagattataccattcaacgcccacatgctgaattgggccattcattataactccaggaacatggcaatcagtaagagcccacatgtttctttgaatacaccgtaagtgaaagaatataaagtagtctagttaatattatgtttaatcaaggagcacattcctaaagatgtttgttcattcattctacagacatttttcagaggcctgctaagagtcaagcattatgatagacgccatggataaaagcttacaagtcaaccagcgtgggtataaataatgtgactagcactagaacaggtatcatgatggggattctgagtataaatatttttttaaaataaatttccccgtgttatttttggcttttacctccctaatttaggctttctaaatggcacagcatttctgaggatgcaaacacctttctacagagcaaaaacagcatttgtataaatttgtgtctttggggaaccaagagactttaaatgtgtttaaaccaataattcagtcaatatcaacattagcttacatgtaatattctcttgatagcccaattttttaaaacactgtattcttagaagtttggtttctaagatgtcactttaagctcttttgcttgttgcttttgtgggatccacaaattttgttctcaggtacataaatgaaggttagtatagaggataaatattatgattcttatctgggaaagacaggtgctgaggtgtaaaagagaggatcctcgccacccatgccccgcaccccctcgccccctgcacccacggatgtgcagtcttacctgcggggggaaaggtctccgagcctggcctgctgctccagctcagggttccccctttcatgatggctttcaaagatttcttcactctgaagtgaaagaaattttggtaagatttgatattgtagggacctcctaatctatatttttctctctcccaatttctgtgtttgattttggttttgagtctctcgataagcaaaatatccagtttctcatgccgctttctcaggttttccccagccactctggatccattatggtttgccctttttggcttcctttaggcacactaaaaactccttcccaaaagcaggtatcggccgggcgtggtggcaggcgcctgtaatcccagctactctggaggctgaggcaggagaattgctcgaacctgggaggcggaggttgcagtgagccaagatcacaccattgcactccagcctcagcaacagagcgagatgccatatccaaaaaaaaaaaaaaaagcaggtgtcctttccccttaatcatgaagggctattcatactttactgccccacccctattgattcataagaggacagtaaagcgatcactgcattcaacatctcctttttttttttcttgtaagaaatcaaggtctggaaaagttgcactcgccctgagaccagaaagtgctggaggcagagatgaagtaagcccagtgtaggctttcacagatgcgtgataacaagtctaactaaagaagtacctgggatactagttttgccaattcagctctaaaacaatatggcaatcttatattccaaatatatatatatatatttgtatttatagtagagatggggtttcaccatgttggctaggctggtcttgaactcctgacctcaaatgatccactgacctcagcctcctaaagtggaggaaacatatatatatgttttccttttaaaataggatgtcagtccaataagaatctaaattttagttccctctaatatatatatctgactagggaccagataatatttttcatgtgtcaatatataaaagttggccaggtgcagtggctcatgcctgtaatcccagcactttaggaggctgaggtcagtggatcatttgaggtcaggagttcaagaccagcctagccaacatggtgaaaccccatctctactaaaaatacaaaaattagccgggcatggtggcaggcacctgtaatccagctatttgggaggctgaggcaggagaatcacttgagcctgggaggcagaggttgcggtgagctgagattgcaccactgcactccagtctgggcgacacagtgagaccctgtctcaaaagaaaaaatatatatatatatatattttttattatattgtttattaaacaaataaaaataaaagatatctcatcagttacgatgtaaatgaaaaaattgtgtgtgtgtgtgtgtgtgtgtgttatgatgtacttcttgtgagttgtggtcaggctttgaaagccactatgtatgtgtgtgtgtgtgtacataaaagtaagtatcagtcccaggattcaaattcgaaccagtcccagtcaaattcaatctaattattttcaccacactaccaaaagtctttcttcacattttctatataaagttgaactaattaatacagcagtgaatgttacattgtatccttttgcagtttctcatctatgacattactatgcctgaattccccactggactttgaactcagtcttactcatctttggatccccagcgtgtaatacaggcccttcataaagagtgaccattattactaacaaaatttcctctcactgtgaaacctgctcccaattataaaatgaattgtgctctttcgaactccgttgatctattgtatgcagggtggatcatagagtcttatttcataataacctagtgatggaaaaataacaatgattcaggagtaggtaatggaccttgtcatcttttacactgaatgatgagatttccctaatttatagattttgtcatgcatgagatgccctcaggagcttgcagaaatgccttggcacgttgctctgctgacatgtgtcagatggctggtgtggaggtagagggagtctcctctgccaagcaagtctaatggaatataacactggtgccattgaggatgcaatgagaaggacccacagcagatccagagactgcattcataaaagctgcacagtataccatgttttattaaggtatagacagattagttgtgttctagcatagtggttttcaaagcctgaccacaactcacaagaagtacataacacacacacacacacacatacacacacacattttttcatttacatcgtaactgatgagatatcttttatttttatttgcttaattttttatttcttttttttttgagacgaaaggtgaattgtagccaaagtacttttaggaaattttaaaattacacattttgaaagctctaggaggaagagagcatattcaagtaaaacctttcttttattcaaatagtagactaaaaaattgaccatagactaactcagcaattgctgccatatttcttagtgagggattccttatgagctccattgaatgataaaaggatactcttccaaatcagagcaacaatcgtttcttgtgacactgcatcctttttcccttccttcaccctgaattgcccctagggtatacctaggagagggagggctggcaaccagctgctgttgtgaaagggatgtcattcatggcctgccctattgggaggccaatcaggcggaacagcccctctccccttatggtcattttatcctgaagaaaaagggtggcaaaaagatggcaaaaaagattgctgatcacagatcactgttacaaatacaacaattggctgggcatggtggctcacgcctgtaatcccagcacttcgggaggccgaggcgagtggatcacgaggtcaagagttcaagaccagcctgaccaacatggtgaaaacccgtctctactaaaaatacaaaaattagccgggcatagtggcgcatgtctgtagtcccagctactcaggaggctgaggcaggaaagtcacttgaacccggaggtggaggttgagtgaaccaagattgtgccactgccctccagcctgggcgacagagcaagattccgtctccaaaaaaaaaaaaggagggggtggcaatgagacagggaactgctctgggtccaactcccctcacatagccccaacccccatatccaccccacaaacctccacctccccacttccagcctctccagtttcaaagtgacgcttaccaacacgcaggctctccaggagcaccttgaaaggtaacttcttacatctttccaaaacacttataatctaattgttctttctcccctatattttgaagaaatgcatgctctcaaaattgaaaaaaacgcctagaggaattttgtaaaaaaattatatttttccagtttctccaaaggacatataaaccctttttaaacttagattttgaaaagtcccaatagctgattttcaatcgattattgaaattgttttttttttcttccctaaaggggagataccaacctttgaacaaaattaaattaacttttccccaaagttaaatgattttactttgtgattttaggaatgtgtagaaagtgatttcagttccaactcttttaaagcaagggtatctgggccaggcgtggtggctccctcctgtaatcctagcgctttgggaggccaaggtggggcagatcacctgtggtcaggagttcgagatcagcctggccaacatagagaaaccccatctctactaaaaacacaaaattagctgggtatagtggtgcgcgcctgtactcccagctactcgggaggctgaggcaggagaatcgcttgaacccgggagacggaggttgcaatgagctgagatcacgccactgcactccagcctgggcaacagagtgagtctctgtcaaaaaaaaaaaaaaaaacaaacaagcaaacaaacaaacaaaaacaagggtatctggtaatttaaggtgaaagtattattcactattataatcaattttcttaaaaacaggagctattaggcccaattctgaggagagaaaaaaaagcagatttcaagggcccgtgccaccttctttgtttaaagcatagagaatgataataaaatgttaaaggttcagcattttctggcattgtaaatttaattaattaattatgtaaattaattatggaaatgagaaataaagtgaaaaggtatgtcaagtaagaatcaataattatcaaaacgttctctaccaaatgggatgcaggggagaagggtgagaagaagggaaggaaataaaggcaaaggcatttgcccacagcctcagtccagggtgttctacatgacgcagccccagagtaaaaaatactagccacaggaatgagtggctctgtcttcctctccagaactgacgggcaaaagagggggaacctcaccttttttttttttttttttttggaaactcactgggttttgatatggagaacaggaagagaatctaccctagcagcacattcacagatggtttgtctgccctatttgctccatctccctggctcttcctagcacttcttttcctactttccattttcattggccgcaaaagccacttaaatattcctttggaaaactgggcagtctgtcccctttttaaaaccacaactatttccaggagagttaaagggctttcggactgcccatcattcatgcattgtggtgaactagctcagactgcctctacctttagtccagatcagctgccttgggagggcgattcctggaggaaaggatgaggagaggaaggttttaggacatcgtatggactgaagccccgttggaggggaagggcaggcttgaggaacaagcctcagtgttcctgaggctttgtggaaatgagaaatgaatagagaaagataagagactggatcctaaatgcaattttgcctatttcaaaattttcacagggtctttttttttttttttttttgagacaaggtcttgctctgtcacccaggctgaagggcagtggtgtgatcatagcttcctgaagcttgcagcctcgaactcctgagctcaagcgatcatcctgcttcagcctcccaaagcgctgggattacaggcatgagccaccacccccagcctcagggtctttttaatccctaagatcttaaatttcccagctactattgttagtaatgagttcaatttacttactggttaaattggcttttattattattattatttattttattttattttattttatttgagacagagtcttgtgctgtcgcccaggctgtggtacagtggtgcaatctcagctcactgcaagctctgcctccctggttcatgccattctcctgcctcagcctcccgagtagctgggactacaggcacccgccaccacgcccagctaattttttgtatttttagtagagacggggtttcaccatgttagccaggatggtctcgatctcctgacctcatcgtctggactaatactctaaacgctattggcaatagtttgtttacaggaaaaacatcttcttataaagctgactgcaaatgttttaataaatttgcaaatatgtaattctgtttaaaatatcaggaagtgagaaacatgttatgtgataactctttcccattcccgaaccaagaaaatgtaaaggcagtaagtgtgccaaggacactgaaaggaagctgcccgtacatctttgaatcttctcaggctgtttggtttcatctgattttaagctccatggataaatttcattgtaacaatttttatgtctgtaaatcttaacccataaataaaatcacaaatcagaaaagtactttattaggccagtctttgtccaagcaaatttggtcccaaagctagtttacaaataattgccacacagctacaaggccagtgggttgactatcgtagcatcaatggtgtgggcagggctgcagcctctggggcagcattagccccaactgacagagggtataggtgctcttaacaatctatgagaccccccacaacctggactcagctgtcataagcctttacttcttatgttcttcctaagtactttatcgggccaacaaattcatcccatgaggaatccaagatggaaacgtcaaaactatctttggtatcatgcttcctccccctaccttctctgccttgatcccctccttcattctacacattttctctgatagtcgtgttctagccactagggagaacgtttcagtcaatgggtatgatttctgctcacctctttttgccatagctattagtaagctctcaccactgatgtctcagctgactgtgacaactgccagcggctggtcagcctgcctgccttcatctaacctgctctttactgtctaccagagtcagcttcgtaatacacaggtgccctcctcctcctgtcaacattcctcaaccaaagcctcagtaaactgtctctcatctaccttcccagcattcttgagccattcccaggatagcctaggccctttagacctctgagatgttatacccttcctcattctggaatgcccttcagtgtcttacccacctttcagagttctatttattctttttttttttttagatggagtctcactctgtcacccaggctggagagcagtggcaccatcttggctcactgcaccctctgcctcccaggttcaagtgattctcctgcctcagcctcctgagtagctgggattacaggtgcccgccaccatgcccagctaatttttgtatttttagtagagacgaggtttcaccatgttggtcagtctggtcttgaactcctgaactcaggtgatccacccacctcagcctcccaaagtgctgggattacaggtgtgagccaccacgaccagccttatttattctttagagttgaattcaaatgccacctcctttgaaagccatccttgatgtccttgtgaggaattactttctctaccacattatatattccttttactgtagaaacgtcattctgctttatattataaattattagaaatatgttgtttcccccatgaaagtttcaagtttcctgagtttaggcctgatgatacaatcattttcatatgactcacagcaattgccttagtatatattaaattgagctgaataagccatgtacccagttgaacacaccaaatattttagtgatgtcatttctttattggtgaaagagcaaggccaacgttgatcttaatgttattctctcttttatcttattcagctccctcaacaaaaatacattctcatagattaatgtaatcaccacctcactcctctcaactttagaccctatactagcctgagaaatccagatccaacttaatgagttctcctttatcccaagtcctcatgacctataaatgtaacctctaggacaatgttacaaagagtgagatctttataccacctgcattcttgtcaccaaacgtgtaagttaaaaattcagattctgagtgtggtgactcacgcctgtaatcccagcactttgggaggctgagccaggtgggtcacctgaggtcaggagttcgagaccagcctgggcaacatggtgaaaccctgtctctactagaaatacaaaaattagccaggcatggtgacaggcacctgtaatcccagctactctagaggctaaggcaggagaatcacttgaacctgggaggcggaagttgcagtgagctgagatcactccatggcactccagcttgggcaacaaagtgagactctgtctccaaaaaaaaaaaaaaatttagattccaggacccgaccctacacctccctaattagaatctcagagagtggggccctgggaatctgcataattcataaccttctcagagctctggtacacaagaaaccatgagaatctttgttctggaaacttcagagaacttggctgagggccaccccaggatttggtggcgtccctgaatctcccattagctctgacactgactagacttcaaatatcacagaaggcaagcattgaagtgtgtttatattcaaatagttttcttgttgagaatcagaaatattaataaaacttttgggagtcaaagtaatgagaagacaaggaactgaacttgccccctgatctgttatcatttggcagtaaggtacatattcaacaataggataattctgtataagacctgaatctgaccttctttcatctttcacactgacaattctgtctctaaacacaggattaaatgaggaactaaaattcaccaagatcctgctaggtgtcaggtacagtgctagctgcttccacacatttctcatttaatcagccacctgtgaaattgctatgatttatcaccatttggcagaaaaggacatttagggcacaagagattaaataagctgctggttgggtacattaagtattaataaataatcagatagataagttaggtagttaagtaagctgccagattagtaaacagctagtttcaaatcccagctccaacatttactagttgtatgacccttggggaaactcaacctctgagtctcatggcactgctgataaaacggaaaacatcatcctcacaaggttctgaggactagggatggactcaggtacacacagcacccagtacaatgtctggccttcagccagtgatagcaacccttcactcacctctccattcccttccagcccctgcattcaaaaacttttatttatttatttttttaatttaggaggctctttgctcagaatcctgaaaaggcttttgttactttattttttcttttttttcatcttctgggagcacagaataggcttctgctcctttaaataactttaacaggcaccaaaggaacactgctagctctttcttaattctgtaggtccacatttaggaaaaagaaattgtcagcctctgacttatttccagcttacaaaaaggctgtgatgttggcagcccggggaaagcagttcccgtgagtcatgctttcacctttcagccagtgaagaacaggaaatagtcacatcactattgccaacaagcacagcgaatcgcttccaccaccgggctttcccagatgacgtcagtgagccaagtgcagggcatcacccttgccagatggccccggaagagtctcagctgccctgtcaagtttagcttctcaagctccccagaaccagcatggcaaggatcacccctccagaaaaggaaatgatttctctgatcatgatcaaaccatgtcataattttagctgtaggtggtgagtatcagtgactgtattatagatggttttggttcacagctattttttttctcggtatatttactctcaagggcaaaggggtggcatttatcacaatgctatgattcactctactagactggctgggtttcatttcatctttgagttctagacctaaggccaaaaggatgtcagaatcgccacccagagctagatgcatgctcaatgcaacctggccatctctctcggacagtggccactaaacacaagctgcaatgattattgttgatgttagtcaattgatttgtcccccttttaataatccatgatccacacagacatgaatctaaaccttttttttgaacctatctttttcttactagttttcttacatattttggaataacaatttccctggttataatacacaatgtgtgaaacactaattgcatttattggtcctaaattcattcccaagtctaggatttggggatttagtgcacaaatccactttcttcctgctcatgttgttcatgactttatggattctgcttacatttcctcccaaagccttggtctttctaagcagaaatctgaccagcctctgttcctactctgcttcctccaccaccttgactgttaacatacactgttgtgggagaagcttttcattaaatacgcaaacaaatcaacaataaggaagaaaacaactgaagcacagaaggatgattgtaacatggacttgtgcttgtttaacagtacccatactgtttggtgagttgcctaaatacagagggagatggcaacttaacaggatgggtttggagtctgaattaaataaacaacatggcaaaagaaggaagaaaccaaaagttgggtgactagggcttcttggattctactcaacactttccacacctacaggcctctgattctatccccaccgctgctcctacactgtctatgtttctcttttacaacaacaacaaaaaaatagcacttgtttactgttcacaattattgaaataaatcactctaatttggattcctttatatgagaacttccatatgcttaaaactcccgtagcatttttatagtgtacaggtcataaacatgttgtctgaagatgggcaaagtaggtcgtagagataggaagtgagttcacatcacggcgtgagtcaaggcagcagcatatgagctcaggggccaaatctgagctgtgatcatgtgacacagtcctcaccttcagagatgacattctcccagaagtccagggtgcagaaatggataggctgatgttcacttttgaagaggaccacaatgaaacccttttaatattactcaatgattccacagacctcctgggtgttaaatcaggtgaatctatttttaaaaattgctatttacttttttaggtggctgatcaggacagtatgaaatttcatacagtttcctaacttgagaaaacatggtgatgcaattcctccaaccaggagggatttatggacagcggtggctacgtcctaatctgcccagaatacaggatgactaaacaaattggcaaacggacgcagctttctctctctctaagaaaagtctgctgagaaccctcgttcccactctgtttctgcctccaagaagaaaagcctaaaactcactttcttccggactctttcaaggtcagtggagttcctctggggttcatattcagatactttggggattgatgatggatcataaatgttgctgaagttcatttgagcccatggcctctggtttcagaaccattcagccagtcaaatatttaaattttagtgaggcaggggaaagggaaccccttcgggggctgctatacagcaacttatattcacaattcacaattaaatacacttcagtttctaaatatatgttactttaaaattatacagtgcttagcaattttcaaattttcattttgtgctcaaaatattttcaggaggtagctgttgttatacccatttgtacaagcaagaaactgaggtttcaagaggttatgttgcttaaacccagatctgcctgattccaaaccttatgctttttttaaacttctattttgaaataattatattaagtcacatgaggttgcaaagaaatggaaagtctcatgtcccctttctccaaacctcatccaatgttaacatctaagaaattgacactggcacaatccacagagcctattcagggttcaccagttattcacgcacttgtgtgtgtgcatgtgtgtgtgcagctctgtgcaattttgtcatgctcttttttttttttttttttgagacatagtttcgctctgttgcccaggctggagtacagtggcacagtctctgctcactgtaacctccaacttccagtttcaagcaattttcctgcctcagcctcctgagtagctgggattacaggcatctgccaccacgcccatctaatttttgtatttttagtagagacaaggtttcaccatgctagccagactggtctcaaactcctgacctcgtgatcttcccacctcggcctcccaaagtgctgggattacaggcgtgaaccaccgcgcccggcctgtcatgctctttttatacatgaaccatacactgttctgccaattttaaatatgaaggcagattacagaaataaataaaatgatttttcttttactacaacagtatctaccaataatcacatacatgaccaaatagctttcacttctagctgccgttaagaagaaagaaaaggatgaaaagaaaaaaaatcctataaaccccagttcttgaaagcattagtctgtgctatgtgccttaggtacagattaaggaaacacaatttgttgatttattgataattgtgacagcaatcttccctcttgtcaggaagttctataagtaaaataaaggtaattttaccttgacttcaratttagtctcatcatcttcctttccccgggagttcaactctgtctccgtataaggttcctcagacgttctcagaggacgagctagagggactaagagaacctgccattagtgtggttatttaacttataaactataactcagtgcactttgtctgatttggacaaatagctggaccacgtcaatgtggctaactataaaagctcacttgagctgctgcgttgatttcctagggctggatgaaattggaggtgaaggagagacacagaggagaaatagaatccttgactccagggacttgacatttcagcagaagagaaaaaagtcacacccagcaccatatgaaacattaaactacaacaaatctgtacatatagcaatgtggtaaagaatagtagattttctgtcattcccatttttaaggcccagctaaaaggtcacttttggctgggtttggtggctcacacctgtaatcccagcactttgggaggctgaggcgggcagatcacctgaggtcaggagttcgagaccagcccggccaacatggtgaaatcccgtctctattaaaaatacaaaaattatccgggaacagtggcaagtgcctgtaatcctagttactcgggaggctgaggcaggagaatcacttgaacctgggaggcggaggttgcggtgagctgagattgcgccactgcactccagcctgggcaacagagctagactccatctcaaacaaacaaacagacaaacaaacaaaaaggccactttcctcatgaagtcttccatattacaaactttatggattcttttatgcactgctgctttggcatgcatcatgtgtacagcataatgttgcacttgatgttgttcttggattcaaagactaaattctaggtactacattgtattgaatatattttccattataaaggtaattaatttttgcttattacagaaaatctgaaaaacactaaactcttaacgctgaagcacaatcacttagtatatttccttacagtcttctttcaaatgctctccctctttttacacacatacatgtacacacacaatcatactatgattgtattgtaagaggatggccatgcctgctgctcttgctcttttctcttggccactctatctcccttctccccacctgggaaatatcctttcttcaagctccatggccatttggtagtaaaattgggacttgagaggagaaggcctcaaggcttcctaacccaccttaccagctttgccaagcattgtgggtgatggccacaaggctaatagataagaggtactttgaattcttatttgtcacagtcaccacccttgcatatgctggtcccttgggaaactcacaggagacatgattaatcccaggcaggatttgagcatttctttctttctttctttttttttttgagacagagtctcgctctgtcgccaggctggagtgcagtggtgtgatctcagctcactgcaacctccgcctcccaggttcaagcgattcttctgcctcagcctcccaagtagctgggactacaggcgcgcaccaccatacgcagctaatttttgtatttttagtagcgacggagttttaccacgttggacaggatggtcttactctcttgacctcatgatctgcctgcctcggcctcccaaagtgctgggattacaggcgtgagccactgtgcccagccaattttagcatttgtaggtcccaagaccaaaatgccattcattgaatgccaccaatatatcaccctgattcttagctatggtagacttgatggaggttgaccatctgatctccagaggccccttccaacccttccattctgtgagccaactggaagcagcttgggctttctatgggtttttcatagatgttatgttgaaaatcgcagaaaattacactgctaccccaggttatacatattaccattaatgctgctttgtaataacagacaatcctttgggctcctccctctctgtgggatctctataaagtgagtgattccaggcacaataactagatgctagaaatgcatgcactataatttgtgacaccatgactgagaagcgcctgtccttgacactgtaagtagagtgaagcagtgaaaggcggggccacagaagccacactgccagtgttcaaaccttgctcttccatgcacgagacaaacatgattacagtcacctctgtaccttacctttttcatctgtaaagtagggaaaaatgagaaatcctacagtaaagggctgtgataaggctttaatgagttcatttgtgaagaatgtagatcatatacaatgcataacacattttagctattattgccacatggcactaaaaagttttttcacgttccttatgaacttcagaggtgttagaagttgtagccaaggcatccagctaacaagagtcagagctgaaattccagctcttaagcccctccacacatcttctttatactgtgaaatacaattgcttatcttggcacagactcagttactaaggaatcagacaaaaatgtttgaaaatccttttagtgacttgctggagtccacactcagtagatacttaccaaaaacaaaagtgcaggattgttctgcaaaacgagctagttgtttgacttaggtcaatgacaacctgttcagacttgacaaagacctatttattctgggaaggagtgttatctattatgaaatcttgtatttgaaaagctgagaaacaaccctgtaattcctgctttatttacttgaaacaaacatatgtacctggaaacaacgaatgaggccaagtcatagatgtgaacatagtttagcttgaggacagacgaaaggtcaagagaaccaagttctgtttccaatcatctaagaaggatgtctctgtgtatgtttgggtatgtctgtggtatgtgtgtgcaagtgtgtgagagtgtatgtgtgtggatgtgtgtgtgtgtgtttgcagataaaaattcaggaaagaaatacactaaatgttatcaaggcttacttctaaaaagtaggatacatggtggaagacaagggatcgtaaattgtactaaatggagaattaccaaaacaccccacaattttcatataaacaaaaactgaaagaatttattgctaacaggccaggcatggtggctcacaactgtaattccagctcttagggaggcagaggcaggaggatagcttgagcccaggagttcgaaacctgcctggacaatatagcaagaccccgttctccacaaaaaggaaaaaataaagacaagaaaagaatttgttgctagtagactccccaacaataaatactaacagaagctctttattccgaagagaaatgacacaagatagtaattcaaatctataataaggaatgaagaaccccagaaatggtgaataagggggtatgtacacacacacacacacacacacacacacacacatgcacacacacacgtatgtatttctcttaatttgatacatgactatttaaagcaaaaattataataccatagtgggggattaacatatatagatgtgatatttatgataagaatagcacagaggatggatggggagcatatggaagtatgacattgtagtatgtagtatgatttcctatattttgcatagaatggtatgaaactaattcaatagatcatgacaagttaaggatgcatatggtaatccccagtctggtgaaaaacagtacaaagagatatgataacaagccaatagaggaattaaaacaaaatattaaaaaatatttactaacccaaaggaaggcaggaaaggaggaaaagaggaataaaaagcacatgagacatgtaggataacaaaacaggaactctaaatccaactatatcaataatgatgatggatgtaaacataaaaatctaatcactccaattacaaggcagaggtcacagtgtataaaaaggcaagacccaactatatgctgcctacaacaaatatactttaaatacaaagacagaagaatagaaaacaaaaaaaatatgttctgcaaacactaagcatgagaaaactggaatggctgtgacaatatcgcacaagatagactttaagagtatttctagaggtaaggagggaaggagagacatttcataattataaaagagctaatatatctgaaagacataaaaaccatgaatatgtatgtgcttaatgacagaggtccacaatacacgacacaaacaaatgacatgtacatcctgcccaagggcagtttattccaaggatgtacagttgttttaacatttgaaaacaaatcattgtaatttaccatattaacaaataaagaagaaaaaccatatcattctctcaatacatggaccaaataagcatttgaaaactcaataatcattcacgataaaaactctcctaaaaaatagaaatagaagagaattccctcaatctgataaaagacatctgtggaaaacctatagcttacatcatacttaaagatgaaacttgaactcttttcctaatattgggaaaacacacagatgtctgctctcaccatttctatttgacattgtagtggaggtcccagtcattataatatgacaagaaaaaaagtataaagattgaaacagaaaaaagtaaaagcatccctattcacagatgataggattatatttctatatagaaatctattcctattcttaattatatatggtaaattccattcattatagggttaaaaaaatgttaaatgcctaaggcctttacactcaaaacgactgcattgttgagagaaattaaagatgacctaaaaaacataagttataacatattcaggaattggaaaagtcaatattggtaagataatagttctctccaaattagctcatatatccagtgtaatccctatcataatcccagcagaaattgaaaggtgattctaaaatttatggaacaatgaaaaggacctaaaatagccaaaacaaccttgaaaaagaacaacgttggaacatcgcatgacttgatcttaaaggctgactaataagctatggtaatcaggaccatgtgggattggcataagaacttacctattttttaaatctttgctcaaagaaccaattttactactccattgctaataaaacatgggccttttaaaggtcctgaatggggtctattttatgctgttattattatgttattattatgatgcatttgttgttgttgttgttgttgttgagacagagttttgctcttgtcgcccaggctggagggcaatggcgcaatctctgctcactgcaacctctgcctcccgggttcaagcaattctcctgcctcagcctcctgagtagctgggattacaggcgcccgccaccatgcccaggctaatttatttatatatatatatgtatatatatgtatgtgtgtgtgtgtgtgtgtatatatatgtgtatatacatatatgtgtatgtgtgtgtgtgtgtgtatatatatatatatatatttttttttttttgagacggagtctcgctctgtcacccaggatggagtgcagtggcgccatctccgctaactgcaagctctgcctcccaggttcacgccattctcctgcctcagcctcccgagtagctgggactacaggtgcccaccaccacgcctggctaattttttgtatttttagtatagatggggtttcaccgtgttagccaggatggtctcgatctcctgacctcgtgatctgcccgcctcaggctcccaaagtgctgggattacaggcgtgagccaccgcgcccagcctaatttttctatttttagtagagatgggtttcaacatgttggccaggctggtctcgaattcctgacctcagataatccacccgccacggcctcccaaagtgctgggattacaggcgtgagctaccgcacccggcctttatgatgcatttttattattccttcagtagattgtgtgtttctccttgcatccaggcacttggtgttatctaagtgtttatgtcttccatgtctgcactatgctatatctgtagtttgttttaaatagtatctgttaaaacagcgaccattatccattgtagacaaccagtcacagtcctcatggtgctgatgaaacacaaaagccacaaagcttcttttcctcaaataacctacccgaggtggagggctggctggtttttgatgacacccattttggtgaagactcaggatgtggggccacaggttgcactggacgagtctgtttggctgccagtttcatcagactcacttgcctcttgtgaaatatttcctggacatcatccagcctctgcaaaactttctgggctttggcctacagtaacaaaagcaaatcatgatgaacaggtctctctgtatacagcctgaggacacgaagcattccctttctcccaccttcccctactccccccatccctacctccctcaggtgaccccctctgggcatcactatgcaagtcgctgcaggtcctgccatgctccagaattgggtatctaaggattagcctctcctacttgagacccttgaggacaatgactacatcctttcactatggcgtctccagtgcctggaacaccctggcacaggagtttgagaccagcctggtcaacatggtgaaatcacatctctactaaaaatacaaaaattagccgggtgtggtggctggcacctgtcatcccagctacttgagaagctgaggcaggagaatcgcttgaacccaggaggcagaggttgcactgagccgagatcatgccactgcactccagcctgggcgacagagcgactccatctcagaaataaacaaacaaacaaacaattagaatacaatgtggcttgtgtcatgttagacagagcaaagaattttggcctgagtccatggatggacttcagggcattccccagaattacacaaacagttagacaggctggatataatctgtttgttcctccactgcatcttctcttcctttctttgtgcccaggaagctgattaaaagaagctcctaaaccagaggtgcagacaaggtgatgatgtggctctcagaggaaggtgtgctacctacaaccctgctggcttcatggaagaatatgcttctcaaatgcaaatccaatcaagttatctccctgcttaaatccaaactcgctcatgtggcccacaaagcctaccttgcctgcctctctctaacctcacccaaaacacacttccattgctctctaggttccagcaccctgaccttttggtctgcactgtgccaggctccctccagccctgaagcctttgcacatgctgttcctcctgtctggaaaggctctcccatcttgtctattcgttctccaggctcaagtgactcttcctgagagccttctctgaccccagtccaggtcaagttcctctgtcacatgcctgaccctgcatccctccttgacagcacttatatcagcttgtaacacatttttgtgtgattattttgttaatgtcagcctctcccacaaactgtaagctcagtgagggatggaagcatgcctatttttaatcatcatggtatcctcagcactcagcacagcacatggtacatcaggaatgctcataagcattaatagagaaatgactgatttgagtcagacgaagactcagtatgccaggacgttcaggccgagagtctaagaggccgccggagggcttcggaaccagtgagcccactcaattcagcctgctcagtaccctgtgtgtactttatcaggaagaaggtacagtccctccctcccctatccgtttcccaaccacaggatcaaaataacccggaagcattacctttgcatcgagggtgagcagcaactcaaactcgttgtaaaactccttggggctgagcaacgggtactccttgactgtgcccaggaatgtcgcaatgtcgttcaaggcgatatcaaccccttctcgagactggcacttgtctacagcttgggaagccaagaggtagattcctgcctcacaccattggctgaccttttggaaagaaacagtgccctgtgcacttcgcctgaccacaactcagagccaccgtaatcctcagcaggtctgagcttgtaaggtgtctacaaggattcccagacaatgggaaattgtcatgggacggcatcgagtttaaggggtctaaccactgtaaattacactatgagggggacaaacccaaaccagctgatacctgtccacttctcagggaagtcactcggtcagcaaaatgagtctcttccattaacagtaattgctacatctccaggaggagacagctaaatatatatttaattaaagacagggtttcactctgtcacccaggctggagtacagtagtgcaattatggcttaatgcagcctggagaggtctaccccaggctcaggtgattcttccaccttagcctcctgagtagctgggactacagatgcatgccaccacaccaggctaatttttccttccttcctcccttccttccttccttccttccttcctctttctttctctctgtctctctttctttctttcctttttttgagacaggttttggccatgttgccgaggctgctctcgaactcctgggctcaagcaatcctcccacctcggcctcccaaagtgctgggattacaggtgtgaggcactgcacccacccagccttatagctaaatatctcttaggctggtctaaccatagagaaatgtaatcaatggcttttggggtttacctgctagtccactcttcttgcatccgaattggttaccagaacacagggtgattaataagacattagacctggcccttacatttcctgggagaagggcatgtcccttttaacaaaaacacaactttcatctttggattcccaggtgatccctatttgcatttactggcgtttgtctttcgtaaggaagcaagaatgcaaagctttgtaactcagcacagcgatactcaagagtttctggatgctgggaggacattagataggtgatgagttctaattattcaatttttaaaaaagactcattttctttgctcttttgaactccactgagacacttgaacagaaagacagggagcccttatgctacctgaagagttatatgaaaagaagtttccagtgccaatacctctgcatttccatttaccatgtgacctgaaaagcaatataaagtaactatacagagaaaaaagatcaaaatgcaacacgccaaagtggttgtctttctaagtgggaaaatgataggcaacttaagtttttgtcttatttttttcctagattatttacagtgaatatgtaccacatttagaataataaaaaatcataaagatatttcagaattaataattaccattatgtagggatgggggatagtgggatatgtacgtgcccatgagtaacctttttcttcctacatttttggatttccacaataatatgaactcatgctttgatatatgctgtgaaaactttctctttacacatcatgcccagatctgcaaaaaacattaggctgggattttctgtagagttactatggctacttgctttctctctctctctgtctccctctctctctctcaaacacacatgcacacacacacacacacacacacacacaccaggcttataatctctggagcaaaaatggagttgggaaaacagcagctgatcccacagcgagctcagcacacagagcaggctgcacaccagccccttcggagctacttgcttctgaaacaactatatttcattgacattaaaatccctttagcaatgggatgaggtaagcgcctgggtgggtgccccctgccatgcaagaggaacagctgtggggatggagcatgctgactcattgccgtcagccacatgctgcctgggaccagctcacagggaagactccagaaaccgctgctcctgctgactgggaacataccctagaggtcacctccaaagggcacccctctccttgtgggcccctggcatgcgcccttgctgtctatcctgtgaagccttggtgtggaatctggcaggcctgactttaattctggcttcccctgtgtgcaccatgtgtggtcttgggcagtcatttaccccagggtttcttggtgacatccctgcaccacctgcaccagaatcatttagacactttactaaaatactagttcctgggcagtattccagacctatcgacgctgagtcttcagggcagtgcataagggaatctgccatataacagctcactaggtgattctcgtgtgctacattttgagacccagcagagtatcagtttctgcctctgcacaatgaggccattaataccttcctcagaggttgtcttgaggattataaatagatgacataattatgttcctggtaggaatgcagtaggtgctcgatacatggtagggggtataaaatggttgttattacttaagcttctccaaaaagagtcaaaaccctttgcctttatatgagggaagcactctgcaaatatagtgcttggttctctgtacacctgaaagggaatcaagaagtgttcccacagggccaagccaaaacacactcttaaagtccggctccacacagatagaaatgggcttcagttaactttaatgggatgtggtgggatggccagagcactttttaccttcgtgccaggtatacttctacaccccaggatctgcacagtttttaatgcctctcgaacatcttttcacataaagcactcaagcctctcagcaatcacacaaggaagggaggacgagtatgatggaagcttcttcacagaaaatcttgcagacatcatgttcttggctgtgttttgctcaccttgtccagctgtctatgaaactctaaggactttcctaaaatgtcccattttttcttgtttccattgatgaaatcgtcacagaggtgcctgagctccacacaccggggcctgatggcatctgctgcataatggtggctttggatgagctggtccccaaccagtgccagcagcwgggccttttccaggggctcctgcaaagtgaacacccacagccaggcgtcagaagtcagagcatcatgaggctgagagctgcctggtactgtgtgctccaagcctggagggaggctgtggagatgctaacccaggatgaaggctggagaacctgagaaagctcggaatggttccaggcccaggaagttaagcctggcccagagttgtccacatgctgaacaacagcagtcattcattcaaatacctgttgagttctgggctagacatcgaggatatagtgttaacaagcccaaaattttcagattaatggagaattcagatatcaactaaattacacaaatcgttaaattattacaattgcaataaactctagggcagaaaagcatggagtgcacataataagagattttgaatacccctattaactaaaggaacaagaagagggactgacacctgtccttgaggagagtggggggtgagctgaagttgaaacactggtgaaaattggatttggacaactcaaaggtggtcatgacacttaagaaatatgcttgtctaggctgggcgtggtggctcccacctgtaatgccagcactttgggaggccgaggtgggcggatcacctgaggtcaggagttcaagacgcgcctgaccaacgtggaaaaaccccgtctctactaaatacaaaaattagatgggcgtggtggtaggtgtctgtaatcccaactactcaggaggctgaggcaggagaatcgcttgaacccgggaggcggagattgcagtgaattgagattgcgccactgcactccagcctgggtgacagagcaagactccatctcaaaaaaaaaaaaaaaaaaaaaaagaaatatgcttgtctaaaccctttcccaaagcctgtaagggccctgtctacactgaccctgtctgctcaatcacatttcctacctctttctgcatgacacttcgctctaaccacacgtgccaagttttcctgtttgtttaagcattccttcattcatccttctcatattattgagaacctgctatttgccaaacactgtcataggtgctgggagttcattggtaaagaaagcaggcaaaaacccccacctcatgaaatgaagcttacattttaatgggagacagaggcaataaacaagtgcatgtgtaggtcatatggtatcttagacagtgatgagtatgtggagaaaaattagatggtggggagggcagggagtgctggagctagtattttaaatcggatggtgagacagtctccctgagaaggagtactcaagctatgcagaaaagacttcacatagaaagcctgagactaggccgggctgtggtggctcacgcctgtaatcctagcactttgggaggccgaggcaggcggattgcctgagttcaggagttggagaccagcctcggcaacacagggaaaccccatctctactaaaacaaaaaaaattagccaggcatggcagcatgcgccgatagtcccagctactctggaagctgaggcaggagaattgcttgaacctgggaagaggaggttgcagtgagccaagatcgtgccactgcactccagcctgggtgagagctaggctctgtctccaaaaaaaaaaaaaaaagagaaaagaaagcctgacactacagccttagaaagaaagacccgattttaagattggccctttgttggcatctaggaacttagatttttgggaaggtttcctccattccctgatgtgaatggttcatgatccctgaactgtttgtgcaaacagtatggtttatggtgattacttgctttttcttctgggagtctgaaatcttggtacttgccagacagaagctgctgacatgaccagccaccaataaaacctctgggcattgagtctctaatgagctcccctggtagacaacatttcacacatgttgtcacaacccacagacgggggcaggaagtatgtcctgtgagactcccacaggagaggattctaagaagcttgtgcctggtttcctccagacttcacctcgcatgcctttctcctttgctaattttgcttagtactttctcatcttaatagatcataactctaatacagctacatgcagaatcctcctagtgaatcttcgaagaaatgtgtgcatggtcttgggagcgctctgcatacaagctgagacctgaagtcagggacaagccgtgcagtacttggctgggaggtgaaggcgtgtttgacaagtttgggtagcaggaaggaggccagtgtggctgcagcagagggagcaagaaggaaatgagtggaagactaactagaggaaagagggggttccatagccggggtgagaggaaagcccctgggtttggagggaggatctcgatcatgtttttgaaaggaccactcccggtacagtgtccagaataagaggaaatgcagggagactcattaaggggctgctgcaggaatccagccgagagatacggcttacggctggtcaccaaactagctcctgactcgacccttctctggattgttcttccctgggtcatgcagagctgagttcctgggtcacctcctcacagcgcttccctggcgactatatcttaagtcattctctacaacctattcgcccagtcacctaggccctctcccacactttaattctttaacagcattatctctatctaaaaactattttctttatttactttaatgtagaattcctccctcctggctgggcgcggtggctcacaccagtaatcccagcactttgggaggccgagacgggcagatcatgaggtcaggagatcgagaccatcctggctaacacggtgaaaccccatctctactaaaaagacaaaaaattagccaggcatggtggcacacacctgtagtcccagctactcgggaggctgaggcaggagaatagcttgaacctgggaggcggaggttgcagtaagctgagattgtgccactgcactccagcctgggcaacagaatgagactctgtctccaaaaaaaaaaaagaattcctccctcctccactcactcccaccaaaatgtaagttccatgagagcatttagtaggcatgcaataaatatttgatcaacaaaataaatgaatgagccaatgagctacatgatgcggtgacctgcttggctggaactctgtgaagcaatcagcacagatgactgccacattccaaaaggtctccaggagcagagagcccatgagcccccttgccagccacactaagcatccagtgcatcactgcactgctttagctgtttacagtaacacaaggagtcacggtaactccaggtgcgtacatcttgcccctctcttaacttgcactggtttatggtttcaacaatttccaaacccctttcctcttaccctcccagtcattcacgaagccccgcagggaccgtcgccttcttaatgcctttcaaattctgaccccatctctgccacctccttgtcactgtctgctcaggcctcatggcttctcatggggacaactggtctccctgcttttcttcaggctctccttcaaatcatgctccagactgctgacaaaggagcaattccaacatatcttattgtgtctacatcactcccaagcttcaagccctttcctgggcgcccagggtcttcaggagcaagcttaaactcttgagcaatgcacacggggaatttcattatctagctcatacctgggtgtgcctgacttaccaagtcctcttccacttcctcaactccattctctgctgcaaccatactaattatccacttattgttccccaaatattcaggcagcttcaggcctccatcccttctccacctgctgtctgcctagaaagcccatccttccctactccccctcacccatactcctctggggccaactcgatctaaatcatatttcagacttcaccttctccagaaaactttctctgtgcttctcttgatcagagcacctgcttcattatgttgtgaccacttatctgctgcctgcgctatgccacagacacctatggtccagaaccagctcttattcaactgaaaacagtgcatgctgcatcttaggtacttacaaataaatgctgcacaaaacaaagaggagtactggtatagaaacaagtgacacaaatcaagtagtggctagttaaaaggactgaggcaaaaatacccagaacacttgttctaaacagcaaaggcccaggtgcactggttaccacccctacaagatgactggaatactcattccctgggcagtgattttttttttttttttttttttgagatggaatcttgctctgtagctaggctggagtgcagtggcttgatctaggctcactgaaaattctgcctccggggttcaagcgattctcctccctcagcctcccgagtagctgggactataggcgcccaccactatgcctggctaattttttttttttttaatttttagcagagatggggtttcaccatgttgcccaggctggtctcgaactcctgagctcaggcaattcacccgcctcggcctcccaaagtgctaggattacaggcgtgagccaccgcgcctggtctgggcagtgatttttgactctttggttcactgctgtgtcctcagaacttaaacagtacctggcatggtaggagatgaataaatatttgcagaatagatgtcgaatgaatgacagacctttcctagcactgctcagctaccgcagactgaacctagcacatgttccatagcccagaatttatccctctacctctgtaatgttaataacaacagctaacatctactgagagcttaacagatgccaggcactgttctgaatgctttacatttgcttatttccttaactcttcacaacaaaccctttgatacaggtactgttttaccaaagggaagtggagcacaatggcccaaggtgacccaggtaggaaagagaagagccaggcttcaaacccaggcagtcttgctccaggatctggctcagaccaacatacacagagaaaaatggctgcacttgttctgcctaaacttagagacctcatttggttgctcgaacttgtctgactttagtcaaatatagaaacatgagacttggcatgaaagtctctgttgacactttttttttttttaagtccagctgcctaatttattaagaacagggcagaattttctgggtccagggaaattcaccacaggatacttccaaatcaccctgtggtgatcagagcctgcccttccctcagcatacagtatttcacccttcagaattttccagaagacatttgagatgttagagggaagatgtgtgtaacaggtccaaatgggtctagggagaaacgcccataaaggcaactgaggaggccgggcgcagtgggtcacgcctgtaatcccagcactttgggaagccaaggccggcggatcacaaggtcaggagttagagaccagcctgaccaacatggtgaaaccccatctctactaaaaatacaaaaattagccgggtgtggtggagggtacctgtagtcccagctacttgggaggctgagacagaagaatcgcttgaacccaggaggcggaggttgcagtgagctgagattgtgccactgcactccagcctgggcggcaagagtgaaactccgtctcaaaaaaaagggcaactaaggagcaacccggattcagccaccacatggctggaaggtcactattacagaattgaaaggacaacaatagtttctgaccactgactactgaccaccaaccatattgcaagctgaccacatgaagtccctaccacacatctaactcatttaatcttcacaaggtctctgtgaggcaggaattattacctctgctttacagatgagggggaccatattcagaaaggctacgaaacgtgactagggtcatacagctggtacttgttagagccaagatttgaatccatccctatccgacttccaaagcctgtgttcttgtcaccttccagcccacctggcctctacacatgtgctatgaaatcctcctggtgcctgcaaagaaatttatggtgaaacctccaaattgaagcttatttttttagtttatatacctcccactcaggataaatagtcatcaaaatctccaaagtaaaaaagaaagagtgagggaaaaaatggctaaaatcttctgatcacaagcccatctgggatctttcagatgctgaaaattccagaaggctggatcgtcaagtttcagcttagaagttgcaatcagaaatctttattgaaataagtttatttccccccgaggactctgctggtataacagatgagagatgagagtgcctactagggaagaaagtggcgagaaagcgagtgatgctactctatccgggaatctagaacaaacccacagaggatccacagggatggagtgtgtgtatggggaggtgggccagatgaaacaagaaaggcagaaagctaatggttgttgaaattgaatgatggacacttggagttcattgtgccagtctatctgcttctctgtgtgtttgaaaatttcacacatgtacacaaagccatgaccctccaacaagatgacatccttagatgttaatggcctgggttccatgccttcttagacacagcacccttatggggagaggtcaaggctgaaatcaggtggttcctccaccctgtcctggtctgttccctgctggcccctcagcaagggtctcagtacggcccacagtcaaaaccctgcagcaagttctgcttttatagaagaccttctgtaagtggttgaaactactgtttttcagaatcaggaaggagcattcaaaagactgctaaaccctcaaacgagaacaagataaagaacaggctgaatcagcatgcgacaggagcctggttagtgagttgactgaggttacttgtgagaatgatgtcactgtgtggaataaggaagctggcaacaccccagagccagcgtgtggctgtgctgggagggagctggccccttagtgccgagtaaaggacaaactccaggcagatatttgtcagattgtggccagacacagtcttgttcctttaacactttaaaggacaaatggaaagattagccaagccctaatcatctgaagaagccatgtgtctgagaagtctgagggaagatatattgaaaacgatctcagttacgatgagacagctttgtttgatttacgcaaaggcagagacacaggtgtactacatccttcaggaagcaagtttagaaataagatctgcaagtcataggatgtgacaggtctgcaaatcctgcaagtcacaggtcatccacagtgggctagtctagctgcttcaagaatgttgcccacgaaaggtgggtcatgggttcacagagcattcctgtgttccataaccacaggtttcatccctaactctggtcagcaacttttgaagtatataccaaggaccacaagttcaaggaagtgggcagaagaaagaatgtgatgcagtcatgcagatccatcgccactattcaagacacagcccagagcgccttccaagggctttctgttccactgagaataaaattcaaatcctcacaaaagatctcagggtgcaacaggactacctggcctctgctcatctctgttcactcatttcctcctggcccacctgctctagcaatgttccttgaactcaccaagctcatttccactttaggacctttgcacctgcagctcgctcttgctttgatactattccctaagtctttgcatgattacacagatcaaacagatctcagaccaaaggtcgtcttctcaggcctttccagaccattctagctaaagtagcatccccagtcactttctattatatcttgttttcttttttatatcacttgttactatctgatgttattatttatttgattgctttttagtgaactgtctctctccagtagaatgtaagctccatgagggcagattctggactgtctggctcaccgctgcacccacggaacctaggataaggaatacctcttgatgaataaataaccctctggataggtcagtagctactttcatatatatatgaaggcattttttttgatgaggcactttttcttaatatatgaataagcaaaaaaatatagtatctatattcagtaaaaaattattttgaaaaaactaaaatgatataaggttggggctgtcctggagaatctaaggtatctagtcatcatatatatgaaattcaacttttccattgtttgataaatgttcctgatgactcatgtgtctttagcactagtttatctgtcaaatggtcactggtaagagaaagagcgctcgctgtgaagggatgggattcctgaatggggatctggatacacagctgggtttgtgtgttcatgcatgtgcacaagcacctgcagctcagaatctacaaccaacagctcttagagtcagtgtggccttggtatactttgttttgtatcctttaggggtgtgtgtctccaactttaacgtacaagtcaatcacctggggatcttcttaaaatgcagattccaattccagggtggggtctgagattttatatttctaattagcgcttactcaggtgacatctgtgctgctggccagaggctcacactttggcaaatctctagtactgcaaaattgccgtctgagaaacatgcctccagattagctaagaaaggaagcgctaaccacaggggacttggtccccaagacccaaaaatatagagaacctaatttcaaacaagcagaaaggaacttctctggaaagagacgactcagctttcttcatttgtaataaaccaaatctgtaaacacttaaaatggaatcatttctaaatgctgacttgtgtgcggatctgggacactgtttaattccagtatgggactgtaaaatacatattcttttacaagtttcacatttacaactcttgggacatgagaaaggagaaagctttgccaattctgtgtagcaatggaactaacatggaccaatttcccccatctaattgtcttgctccaggtataaaactagttgtttttttttttctggtttctcctttgaaagtttcccatccttactaccaaccccacaacccccagccccctagtccaggctccagggaggagctcaagctgatgggtgagcctctgatataattgttctggtgagtgatgagcctgttgtgaccttctcccacttggctgctgaccacgtgagagggcagggctggtccagagtcctcacccgacctgagcgtccaactgcaactgtatgttccggcagctttaataggactctgtgctgcccgtattattcatagcagttgttaatgtgcctctctccttgggatcctggggtgactgaagggcaggactgggcattcctgtgtcctttagcaaccttccaatagcaatgactggacatgatccatcattgtccacagcaacctccctcccagtacacaaaaatgtcagaaagcaaacctggcttttttcctccagttttttgtgttccttaagaakctgctccacgtgcatcacgctgtctccaatgcctgtaaactctgcttgctcttccagcaaattatccagggcaagcttagcctacaagaaacattagaacagtccagaagtaaaccaacactcactcacctgaaggtataggtttgttatgcttcaagaaaactcaaaattccaaaaaggattcaagacttgtatatatctatatatgttaaaaattttttttttgcagctcactaactttcaaa

SEQ ID NO: 2 - DNA, 3189 bp, Homo sapiens
cgctgaattctaggggaggatggcgcccccatcatcacgttcccagagttttcggggttcaaacacatcccagatgaagacttcctgaatgtcatgacctacctgactagcatccccagtgtggaggctgccagcattggattcattgttgttatcgacagacgaagagacaagtggagctccgtaaaggcatccttgacacgaatagctgtggcatttccaggaaacttacagctcatattcatccttcgtccatctcgctttatccagaggacattcactgacattggcattaaatactatcgaaatgagtttaaaacgaaagtgccgatcatcatggtaaactctgtctctgaccttcacggctacatcgacaaaagccaactgacccgggaattaggggggactttggaatatcgccacggtcagtgggtaaatcaccgcactgccatcgaaaactttgccttgaccttgaagaccactgcccagatgctgcagacgtttgggtcctgcctggccacagcagagctgcccagaagcatgctatccacggaagaccttctcatgtcccacacaaggcagcgggacaagctgcaggatgagctgaaattacttggaaagcaggggaccacattgctgtcatgcatccaagaaccagcaaccaaatgtcccaacagcaaactcaatctcaaccaacttgagaatgtaactaccatggaaaggttattagttcaactggatgaaacagaaaaagcctttagtcacttttggtctgagcatcatctgaagcttaaccagtgcctacaactacagcattttgagcacgatttttgtaaggctaagcttgccctggataatttgctggaagagcaagcagagtttacaggcattggagacagcgtgatgcacgtggagcagattcttaaggaacacaaaaaactggaggaaaaaagccaggagtccctggaaaaggcccagctgctggcactggttggggaccagctcatccaaagccaccattatgcagcagatgccatcaggccccggtgtgtggagctcaggcacctctgtgacgatttcatcaatggaaacaagaaaaaatgggacattttaggaaagtccttagagtttcatagacagctggacaaggtcagccaatggtgtgaggcaggaatctacctcttggcttcccaagctgtagacaagtgccagtctcgagaaggggttgatatcgccttgaacgacattgcgacattcctgggcacagtcaaggagtacccgttgctcagccccaaggagttttacaacgagtttgagttgctgctcaccctcgatgcaaaggccaaagcccagaaagttttgcagaggctggatgatgtccaggaaatatttcacaagaggcaagtgagtctgatgaaactggcagccaaacagactcgtccagtgcaacctgtggccccacatcctgagtcttcaccaaaatgggtgtcatcaaaaaccagccagccctccacctcggtccctctagctcgtcctctgagaacgtctgaggaaccttatacggagacagagttgaactcccggggaaaggaagatgatgagactaaatttgaagtcaagagtgaagaaatctttgaaagccatcatgaaagggggaaccctgagctggagcagcaggccaggctcggagacctttccccccgcaggcgcattatacgtgacttgcttgagactgaagagatttacataaaagagattaaaagcataattgatggatatatcactccaatggattttatttggctaaagcatctaattccagatgttcttcagaataacaaggactttctctttgggaatattagagaactttacgaatttcacaacaggacttttctaaaagagttggaaaagtgtgctgagaaccctgaacttctggcacattgctttctcaagagaaaagaagatcttcagatatattttaaataccataagaatctgccccgagctagggcaatctggcaagagtgtcaagactgcgcctactttggggtatgccagcgccaactggatcacaatctccctctttttaagtatctcaaaggaccaagccagagacttataaaataccagatgctgttgaagggtctgctggatttcgagtctcctgaagatatggagatagacccaggtgaactaggaggctcggctaaggatgggccaaagagaaccaaagattcagcattctcaactgaactacaacaagctttggcagtgatagaggatttgatcaagtcctgtgagttggctgtggacctagcagcagtgactgaatgtccggacgatattggaaaactaggcaagctgttgctgcacggccctttcagcgtctggacaattcacaaggatcgttataaaatgaaggatttgattcgatttaaacccagccagaggcaaatctacctatttgaaaggggaatagtgttctgtaagatacgaatggagcctggggaccagggattatctcctcattacagcttcaagaaggccatgaagctgatgacactttcaattcgccagcttggaagggggagccatagaaagtttgagattgccagtcgaaatggacttgagaaatacatcctgcaggcagcttcaaaagaaatcagagactgttggttttcagaaataagtaaattattgatggaacaacaaaataatatcaaagaccaaggaaatccacagtttgaaatgagcacgagcaaaggcagtggagcaggatccggaccatggattaaaaatatggaaagagctaccactagcaaggaagacccggcctccagcacaggagggattaaaggctgctccagcagggagtttagctccatggacacctttgaagactgtgaaggcgcagaagacatggaaaaggagagcagtgctctgagtctcgcgggccttttccagtcggacgacagtcacgaaacctgttcctccaaatctgctttcctggagaggggagaaagcagccagggagaaaaagaagaacgcgatgaggaggaaacggcgacccgcagcaccgaggaggagcgcgctggggcgtccacgggccggctggctcctgcgggggcgacggctggtttccaggcgagggcgctgcgcccgaggacctccgcccaggagagctga

SEQ ID NO: 3 - DNA, 4772 bp, Homo sapiens
ttgggcggagatgcctttaaaaaatcatccaccgcagcggtagaaacagttttgtttggctttatttatacggaatggtttttcagtgaaatgctgtcttgcttaaaagaagagatgcctccccaggagctcacccggcgactggccacagtgatcactcatgtcgatgaaattatgcagcaggaagtcagacccctgatggcggtggagataatagaacaacttcacagacaatttgccattctttcaggaggccgaggggaggatggcgcccccatcatcacgttcccagagttttcggggttcaaacacatcccagatgaagacttcctgaatgtcatgacctacctgactagcatccccagtgtggaggctgccagcattggattcattgttgttatcgacagacgaagagacaagtggagctccgtaaaggcatccttgacacgaatagctgtggcatttccaggaaacttacagctcatattcatccttcgtccatctcgctttatccagaggacattcactgacattggcattaaatactatcgaaatgagtttaaaacgaaagtgccgatcatcatggtaaactctgtctctgaccttcacggctacatcgacaaaagccaactgacccgggaattaggggggactttggaatatcgccacggtcagtgggtaaatcaccgcactgccatcgaaaactttgccttgaccttgaagaccactgcccagatgctgcagacgtttgggtcctgcctggccacagcagagctgcccagaagcatgctatccacggaagaccttctcatgtcccacacaaggcagcgggacaagctgcaggatgagctgaaattacttggaaagcaggggaccacattgctgtcatgcatccaagaaccagcaaccaaatgtcccaacagcaaactcaatctcaaccaacttgagaatgtaactaccatggaaaggttattagttcaactggatgaaacagaaaaagcctttagtcacttttggtctgagcatcatctgaagcttaaccagtgcctacaactacagcattttgagcacgatttttgtaaggctaagcttgccctggataatttgctggaagagcaagcagagtttacaggcattggagacagcgtgatgcacgtggagcagattcttaaggaacacaaaaaactggaggaaaaaagccaggagcccctggaaaaggcccagctgctggcactggttggggaccagctcatccaaagccaccattatgcagcagatgccatcaggccccggtgtgtggagctcaggcacctctgtgacgatttcatcaatggaaacaagaaaaaatgggacattttaggaaagtccttagagtttcatagacagctggacaaggtcagccaatggtgtgaggcaggaatctacctcttggcttcccaagctgtagacaagtgccagtctcgagaaggggttgatatcgccttgaacgacattgcgacattcctgggcacagtcaaggagtacccgttgctcagccccaaggagttttacaacgagtttgagttgctgctcaccctcgatgcaaaggccaaagcccagaaagttttgcagaggctggatgatgtccaggaaatatttcacaagaggcaagtgagtctgatgaaactggcagccaaacagactcgtccagtgcaacctgtggccccacatcctgagtcttcaccaaaatgggtgtcatcaaaaaccagccagccctccacctcggtccctctagctcgtcctctgagaacgtctgaggaaccttatacggagacagagttgaactcccggggaaaggaagatgatgagactaaatttgaagtcaagagtgaagaaatctttgaaagccatcatgaaagggggaaccctgagctggagcagcaggccaggctcggagacctttccccccgcaggcgcattatacgtgacttgcttgagactgaagagatttacataaaagagattaaaagcataattgatggatatatcactccaatggattttatttggctaaagcatctaattccagatgttcttcagaataacaaggactttctctttgggaatattagagaactttacgaatttcacaacaggacttttctaaaagagttggaaaagtgtgctgagaaccctgaacttctggcacattgctttctcaagagaaaagaagatcttcagatatattttaaataccataagaatctgccccgagctagggcaatctggcaagagtgtcaagactgcgcctactttggggtatgccagcgccaactggatcacaatctccctctttttaagtatctcaaaggaccaagccagagacttataaaataccagatgctgttgaagggtctgctggatttcgagtctcctgaagatatggagatagacccaggtgaactaggaggctcggctaaggatgggccaaagagaaccaaagattcagcattctcaactgaactacaacaagctttggcagtgatagaggatttgatcaagtcctgtgagttggctgtggacctagcagcagtgactgaatgtccggacgatattggaaaactaggcaagctgttgctgcacggccctttcagcgtctggacaattcacaaggatcgttataaaatgaaggatttgattcgatttaaacccagccagaggcaaatctacctatttgaaaggggaatagtgttctgtaagatacgaatggagcctggggaccagggattatctcctcattacagcttcaagaagaccatgaagctgatgacactttcaattcgccagcttggaagggggagccatagaaagtttgagattgccagtcgaaatggacttgagaaatacatcctgcaggcagcttcaaaagaaatcagagactgttggttttcagaaataagtaaattattgatggaacaacaaaataatatcaaagaccaaggaaatccacagtttgaaatgagcacgagcaaaggcagtggagcaggatccggaccatggattaaaaatatggaaagagctaccactagcaaggaagacccggcctccagcacaggagggattaaaggctgctccagcagggagtttagctccatggacacctttgaagactgtgaaggcgcagaagacatggaaaaggagagcagtgctctgagtctcgcgggccttttccagtcggacgacagtcacgaaacctgttcctccaaatctgctttcctggagaggggagaaagcagccagggagaaaaagaagaacgcgatgaggaggaaacggcgacccgcagcaccgaggaggagcgcgctggggcgtccacgggccggctggctcctgcgggggcgacggctggtttccaggcgagggcgctgcgcccgaggacctccgcccaggagagctgacctccctgcggacgcccccgctcctcggctccagagcgcccgcattcccgggagaggcggtgtgggggcccgggccctgcccagctacgcagaaagcagccggagcctcggcggcggcagaaaggggacaaccagggcctcctccgaggagcccgaggggtgtcctgggtgcgcgcctagctccgcacgggggacctcggagctgctctaaggcgcctgcagaggcgagcagagcccgcagcccacgccttctcgaccgcgcacttcgacattcggagccgggcaattctttgctgcgtgggcttctctgtgctcccacggtaggatgtttagtagcacccctggcctctacccactaggtgccaggaatgcgccaccccatcctccaccccgcccccaaatcgtgacaatcaaaaatgcctgcagacacgcccgcatttctccagggcggggtgggaatcggttgagagccgcttagcccgagccttggaggagccggagccgctcaaacccggcgggggccgcagactgggagctcccggtccgcctcccagcatccctgcgagcgttcatggggtgttcgtgttagtgccaagattgcttcgttgtagagagagttcgttccaagttactttctgaggtattttatgtatcgatttattagtttttaaatgagttttgttagtttcagttgtattttatttttagttttattagttgattttcatttctttgtagctttctggttatttaattttatagtttcagttactggtttatagttactttattttcaaagttatttagttgttcattttcagttatttatatgtagttgttttgttttaggagttacagatgttcaaattaatttgcttggaatttatttatttatttatttatttatttttcgagacggagtctcgctctgtcgcccaggctggagtgcagtggtgtgatctcggctcactgcaaaccgcctcccgggttcacgccattctcctgcctcagcctcctgagtagctgggactacaggcgcccgccaccacgcccggctaattttttatttggatttttagtagagacggggtttccccgtgttagccaggatggtctcgatctcctgacctcgtgatccccctgcctcggcctcccaaagtgctgggattacaggcgtgagccaccgcgcccagccggaatttatttttaattatctttacaattattatctgagttatttaccttcatagttatttcactttatcttattggagtttttgagttatttatttttacagtaacttatttgactattaaacactcactggaagttcatg

SEQ ID NO: 4 - protein, 1031 aa, Homo sapiens [sequence omitted]

SEQ ID NO: 5 - protein, 1114 aa, Homo sapiens
MLSCLKEEMPPQELTRRLATVITHVDEIMQQEVRPLMAVEIIEQLHRQFAILSGGRGEDGAPIITFPEFSGFKHIPDEDFLNVMTYLTSIPSVEAASIGFIVVIDRRRDKWSSVKASLTRIAVAFPGNLQLIFILRPSRFIQRTFTDIGIKYYRNEFKTKVPIIMVNSVSDLHGYIDKSQLTRELGGTLEYRHGQWVNHRTAIENFALTLKTTAQMLQTFGSCLATAELPRSMLSTEDLLMSHTRQRDKLQDELKLLGKQGTTLLSCIQEPATKCPNSKLNLNQLENVTTMERLLVQLDETEKAFSHFWSEHHLKLNQCLQLQHFEHDFCKAKLALDNLLEEQAEFTGIGDSVMHVEQILKEHKKLEEKSQEPLEKAQLLALVGDQLIQSHHYAADAIRPRCVELRHLCDDFINGNKKKWDILGKSLEFHRQLDKVSQWCEAGIYLLASQAVDKCQSREGVDIALNDIATFLGTVKEYPLLSPKEFYNEFELLLTLDAKAKAQKVLQRLDDVQEIFHKRQVSLMKLAAKQTRPVQPVAPHPESSPKWVSSKTSQPSTSVPLARPLRTSEEPYTETELNSRGKEDDETKFEVKSEEIFESHHERGNPELEQQARLGDLSPRRRIIRDLLETEEIYIKEIKSIIDGYITPMDFIWLKHLIPDVLQNNKDFLFGNIRELYEFHNRTFLKELEKCAENPELLAHCFLKRKEDLQIYFKYHKNLPRARAIWQECQDCAYFGVCQRQLDHNLPLFKYLKGPSQRLIKYQMLLKGLLDFESPEDMEIDPGELGGSAKDGPKRTKDSAFSTELQQALAVIEDLIKSCELAVDLAAVTECPDDIGKLGKLLLHGPFSVWTIHKDRYKMKDLIRFKPSQRQIYLFERGIVFCKIRMEPGDQGLSPHYSFKKTMKLMTLSIRQLGRGSHRKFEIASRNGLEKYILQAASKEIRDCWFSEISKLLMEQQNNIKDQGNPQFEMSTSKGSGAGSGPWIKNMERATTSKEDPASSTGGIKGCSSREFSSMDTFEDCEGAEDMEKESSALSLAGLFQSDDSHETCSSKSAFLERGESSQGEKEERDEEETATRSTEEERAGASTGRLAPAGATAGFQARALRPRTSAQES

SEQ ID NO: 6 - DNA, 401 bp, Homo sapiens
  misc_feature 201: n = g or c
gtgcctttctgaaccctgtgacccagcagcctccatcaactcgtcctacctgccatgcacagctcctctgtgcccctgtacctgagctcatgctattccctctgccaggatgcccttctccttctccaccaggagaagaacacttgccagtaagacccagttctaatgtcaccccttcctgacggtatcaggaagagtcantgatggtgttttatgctcccagagaatttgccacattgtgttgtgattatttttccacatctgtctcccccactggaatgagagcctcactcatcttcatacctccctggtctctacctggtgccagaaccatcctcagggcaggggaatgctcaggaaatagatattgaataaaataagtgtatccatccatccatcca

SEQ ID NO: 7 - DNA, 20 bp, Artificial Sequence
  Primer
atgtcaagtgcacccacatg

SEQ ID NO: 8 - DNA, 20 bp, Artificial Sequence
  Primer
aggaagaaactgacggaagg

SEQ ID NO: 9 - DNA, 19 bp, Artificial Sequence
  Primer
cacatgcctgctcgccccc

SEQ ID NO: 10 - DNA, 30 bp, Artificial Sequence
  Primer
acgttggatgacgtgtcggtcccctttcat

SEQ ID NO: 11 - DNA, 29 bp, Artificial Sequence
  Primer
acgttggatgacgcgccacacctccctac

SEQ ID NO: 12 - DNA, 30 bp, Artificial Sequence
  Primer
acgttggatgtgggcgaggttctgcagcgt

SEQ ID NO: 13 - DNA, 30 bp, Artificial Sequence
  Primer
acgttggatggtttcgtttctccggcacag

SEQ ID NO: 14 - DNA, 30 bp, Artificial Sequence
  Primer
acgttggatggaggagacccagggtatgag

SEQ ID NO: 15 - DNA, 29 bp, Artificial Sequence
  Primer
acgttggatgtctgggaccgtttaccgca

SEQ ID NO: 16 - DNA, 30 bp, Artificial Sequence
  Primer
acgttggatgcacacattcttctcaagtgc

SEQ ID NO: 17 - DNA, 30 bp, Artificial Sequence
  Primer
acgttggatgggagggacacaatttaactc

SEQ ID NO: 18 - DNA, 30 bp, Artificial Sequence
  Primer
acgttggatgggcaccatgtgtggctaatt

SEQ ID NO: 19 - DNA, 30 bp, Artificial Sequence
  Primer
acgttggatgaaggatcacgtgaagtcagg

SEQ ID NO: 20 - DNA, 30 bp, Artificial Sequence
  Primer
acgttggatggaaggtggagcagacattag

SEQ ID NO: 21 - DNA, 30 bp, Artificial Sequence
  Primer
acgttggatgaccttagttataccaggcac

SEQ ID NO: 22 - DNA, 30 bp, Artificial Sequence
  Primer
acgttggatgacagagaggcttggtcatac

SEQ ID NO: 23 - DNA, 30 bp, Artificial Sequence
  Primer
acgttggatgggtgcttggttgtgattctc

SEQ ID NO: 24 - DNA, 30 bp, Artificial Sequence
  Primer
acgttggatgattcctgcaggtactgtgtc

SEQ ID NO: 25 - DNA, 30 bp, Artificial Sequence
  Primer
acgttggatgtgagcccaaaactactgctg

SEQ ID NO: 26 - DNA, 30 bp, Artificial Sequence
  Primer
acgttggatgaccaccagataaaatccctc

SEQ ID NO: 27 - DNA, 30 bp, Artificial Sequence
  Primer
acgttggatgaagttcctctggtggacaac

SEQ ID NO: 28 - DNA, 30 bp, Artificial Sequence
  Primer
acgttggatgtgagtgacatttgtgtcacc

SEQ ID NO: 29 - DNA, 30 bp, Artificial Sequence
  Primer
acgttggatgcggaggatctgaacaacttc

SEQ ID NO: 30 - DNA, 30 bp, Artificial Sequence
  Primer
acgttggatggggagtcattccaataccag

SEQ ID NO: 31 - DNA, 30 bp, Artificial Sequence
  Primer
acgttggatgggagtgaaaggtcatattgg

SEQ ID NO: 32 - DNA, 31 bp, Artificial Sequence
  Primer
acgttggatgcacaatctgttagaatggtgg

SEQ ID NO: 33 - DNA, 30 bp, Artificial Sequence
  Primer
acgttggatgcgtcaagactgaatgcatag

SEQ ID NO: 34 - DNA, 30 bp, Artificial Sequence
  Primer
acgttggatggaaaatatagtcctacacaa

SEQ ID NO: 35 - DNA, 30 bp, Artificial Sequence; identical to SEQ ID NO: 33
  Primer

SEQ ID NO: 36 - DNA, 30 bp, Artificial Sequence
  Primer
acgttggatggttctaatgtcaccccttcc

SEQ ID NO: 37 - DNA, 30 bp, Artificial Sequence
  Primer
acgttggatgcaatgtggcaaattctctgg

SEQ ID NO: 38 - DNA, 30 bp, Artificial Sequence
  Primer
acgttggatgcaaatcacccctgacaattc

SEQ ID NO: 39 - DNA, 30 bp, Artificial Sequence
  Primer
acgttggatgaccagcacactcagctttag

SEQ ID NO: 40 - DNA, 30 bp, Artificial Sequence
  Primer
acgttggatgcagaaatatgaaggtggccc

SEQ ID NO: 41 - DNA, 30 bp, Artificial Sequence
  Primer
acgttggatgaccttgagctctgagccctt

SEQ ID NO: 42 - DNA, 30 bp, Artificial Sequence
  Primer
acgttggatgctcctcctcagaggactaac

SEQ ID NO: 43 - DNA, 30 bp, Artificial Sequence
  Primer
acgttggatgagcccaaggactcatacaac

SEQ ID NO: 44 - DNA, 30 bp, Artificial Sequence
  Primer
acgttggatgaaacatggcgaaacccggtc

SEQ ID NO: 45 - DNA, 30 bp, Artificial Sequence
  Primer
acgttggatgaccacgcctggctaattttg

SEQ ID NO: 46 - DNA, 29 bp, Artificial Sequence
  Primer
acgttggatgaccgggagctcccagtctg

SEQ ID NO: 47 - DNA, 30 bp, Artificial Sequence
  Primer
acgttggatgtgggaatcggttgagagccg

SEQ ID NO: 48 - DNA, 30 bp, Artificial Sequence
  Primer
acgttggatggcagcaaagaattgcccggc

SEQ ID NO: 49 - DNA, 30 bp, Artificial Sequence
  Primer
acgttggatgtaaggcgcctgcagaggcga

SEQ ID NO: 50 - DNA, 30 bp, Artificial Sequence
  Primer
acgttggatgctgcagttatctgggtgagc

SEQ ID NO: 51 - DNA, 30 bp, Artificial Sequence
  Primer
acgttggatgccagaacgtggcttgttggg

SEQ ID NO: 52 - DNA, 30 bp, Artificial Sequence
  Primer
acgttggatgtggtacctccaggtaaaatg

SEQ ID NO: 53 - DNA, 30 bp, Artificial Sequence
  Primer
acgttggatgtccaggcagtcattttaccc

SEQ ID NO: 54 - DNA, 30 bp, Artificial Sequence
  Primer
acgttggatgttctctgcggaggaaagtgc

SEQ ID NO: 55 - DNA, 30 bp, Artificial Sequence
  Primer
acgttggatgttaagccagtccccacaagg

SEQ ID NO: 56 - DNA, 30 bp, Artificial Sequence
  Primer
acgttggatgatcacttggactcaggaagc

SEQ ID NO: 57 - DNA, 30 bp, Artificial Sequence
  Primer
acgttggatgagtcttgctctgtttccagg

SEQ ID NO: 58 - DNA, 30 bp, Artificial Sequence
  Primer
acgttggatgtcacctgagcatcagacatg

SEQ ID NO: 59 - DNA, 30 bp, Artificial Sequence
  Primer
acgttggatgatagtggaaggagaaacggg

SEQ ID NO: 60 - DNA, 30 bp, Artificial Sequence
  Primer
acgttggatgaagcctcagatgaggcatac

SEQ ID NO: 61 - DNA, 30 bp, Artificial Sequence
  Primer
acgttggatgtctgaaagggttcaggaagg

SEQ ID NO: 62 - DNA, 30 bp, Artificial Sequence
  Primer
acgttggatgcgttgatgcactgaagggag

SEQ ID NO: 63 - DNA, 30 bp, Artificial Sequence
  Primer
acgttggatgagaggctaaatgttggcagg

SEQ ID NO: 64 - DNA, 30 bp, Artificial Sequence
  Primer
acgttggatgcaattgctcagaccttcacc

SEQ ID NO: 65 - DNA, 30 bp, Artificial Sequence
  Primer
acgttggatgaatgctagagacattgcacc

SEQ ID NO: 66 - DNA, 30 bp, Artificial Sequence
  Primer
acgttggatgtgaggacctcattattggtg

SEQ ID NO: 67 - DNA, 30 bp, Artificial Sequence
  Primer
acgttggatgctgagcaatcgaactgctac

SEQ ID NO: 68 - DNA, 30 bp, Artificial Sequence
  Primer
acgttggatgctagaattacaggtgcacac

SEQ ID NO: 69 - DNA, 30 bp, Artificial Sequence
  Primer
acgttggatggccaagatggtgaaaccttg

SEQ ID NO: 70 - DNA, 31 bp, Artificial Sequence
  Primer
acgttggatggcattttaccaccatgtggtt

SEQ ID NO: 71 - DNA, 30 bp, Artificial Sequence
  Primer
acgttggatgccttcatgttaattctgccc

SEQ ID NO: 72 - DNA, 30 bp, Artificial Sequence
  Primer
acgttggatgctttactgagtgggcaaacg

SEQ ID NO: 73 - DNA, 30 bp, Artificial Sequence
  Primer
acgttggatgtctaagtggaactcagcagc

SEQ ID NO: 74 - DNA, 30 bp, Artificial Sequence
  Primer
acgttggatgaatatcctaggctagcagtg

SEQ ID NO: 75 - DNA, 30 bp, Artificial Sequence
  Primer
acgttggatggtgcataaatacatgaatag

SEQ ID NO: 76 - DNA, 30 bp, Artificial Sequence
  Primer
acgttggatgtccaggtgttatggagtcag

SEQ ID NO: 77 - DNA, 30 bp, Artificial Sequence
  Primer
acgttggatgggcttcttgtgctgctgtgt

SEQ ID NO: 78 - DNA, 30 bp, Artificial Sequence
  Primer
acgttggatgtcaacaaagatgccaagacc

SEQ ID NO: 79 - DNA, 30 bp, Artificial Sequence
  Primer
acgttggatggtggatatccattgtcctag

SEQ ID NO: 80 - DNA, 30 bp, Artificial Sequence
  Primer
acgttggatgggctgagtaacagtccattg

SEQ ID NO: 81 - DNA, 30 bp, Artificial Sequence
  Primer
acgttggatgcttacagtatccaaaaaggg

SEQ ID NO: 82 - DNA, 30 bp, Artificial Sequence
  Primer
acgttggatgtcaaaggtaggttacccctg

SEQ ID NO: 83 - DNA, 30 bp, Artificial Sequence
  Primer
acgttggatgatccccaatttgcacatccc

SEQ ID NO: 84 - DNA, 30 bp, Artificial Sequence
  Primer
acgttggatgtgcagccctcaacctttcag

SEQ ID NO: 85 - DNA, 31 bp, Artificial Sequence
  Primer
acgttggatgccttattcagttactattacg

SEQ ID NO: 86 - DNA, 30 bp, Artificial Sequence
  Primer
acgttggatgaaacacacacacccacatac

SEQ ID NO: 87 - DNA, 30 bp, Artificial Sequence
  Primer
acgttggatggggagaaagaaaacaaaggc

SEQ ID NO: 88 - DNA, 30 bp, Artificial Sequence
  Primer
acgttggatgcaatgcctgcacttagacac

SEQ ID NO: 89 - DNA, 30 bp, Artificial Sequence
  Primer
acgttggatgagtgatgagaacatgggctg

SEQ ID NO: 90 - DNA, 30 bp, Artificial Sequence
  Primer
acgttggatggcaataggactccctttacc

SEQ ID NO: 91 - DNA, 30 bp, Artificial Sequence
  Primer
acgttggatgaagatacgaatggagcctgg

SEQ ID NO: 92 - DNA, 30 bp, Artificial Sequence
  Primer
acgttggatgtttttgagcttcactgagcg

SEQ ID NO: 93 - DNA, 30 bp, Artificial Sequence
  Primer
acgttggatgcgtatctctagctcaagcat

SEQ ID NO: 94 - DNA, 30 bp, Artificial Sequence
  Primer
acgttggatgcagaagttaggatgcagatg

SEQ ID NO: 95 - DNA, 31 bp, Artificial Sequence
  Primer
acgttggatgccagtagagatagaattttgg

SEQ ID NO: 96 - DNA, 30 bp, Artificial Sequence
  Primer
acgttggatgatacctagagtttgcccaac

SEQ ID NO: 97 - DNA, 30 bp, Artificial Sequence
  Primer
acgttggatgagctgagatcaatccctatg

SEQ ID NO: 98 - DNA, 30 bp, Artificial Sequence
  Primer
acgttggatggtggcagtcaaaacacagtc

SEQ ID NO: 99 - DNA, 30 bp, Artificial Sequence
  Primer
acgttggatgacagagtaagactccgtctc

SEQ ID NO: 100 - DNA, 30 bp, Artificial Sequence
  Primer
acgttggatggctattagaaagtcagagcc

SEQ ID NO: 101 - DNA, 30 bp, Artificial Sequence
  Primer
acgttggatgtgttccagaaggtgtagaag

SEQ ID NO: 102 - DNA, 30 bp, Artificial Sequence
  Primer
acgttggatgcttcagttcagggagagatc

SEQ ID NO: 103 - DNA, 30 bp, Artificial Sequence
  Primer
acgttggatgatagggcccccagcataaaa

SEQ ID NO: 104 - DNA, 30 bp, Artificial Sequence
  Primer
acgttggatggctttcccctaaagcatctc

SEQ ID NO: 105 - DNA, 30 bp, Artificial Sequence
  Primer
acgttggatggatctctccctgaactgaag

SEQ ID NO: 106 - DNA, 30 bp, Artificial Sequence
  Primer
acgttggatgtattccactgcctgctttcc

SEQ ID NO: 107 - DNA, 30 bp, Artificial Sequence
  Primer
acgttggatggaaaacaggaggaagtggtg

SEQ ID NO: 108 - DNA, 30 bp, Artificial Sequence
  Primer
acgttggatgatgtcaagtgcacccacatg

SEQ ID NO: 109 - DNA, 30 bp, Artificial Sequence
  Primer
acgttggatgaggaagaaactgacggaagg

SEQ ID NO: 110 - DNA, 30 bp, Artificial Sequence; identical to SEQ ID NO: 108
  Primer

SEQ ID NO: 111 - DNA, 30 bp, Artificial Sequence; identical to SEQ ID NO: 109
  Primer

SEQ ID NO: 112 - DNA, 30 bp, Artificial Sequence
  Primer
acgttggatgtctcaccttgcctttggacg

SEQ ID NO: 113 - DNA, 30 bp, Artificial Sequence
  Primer
acgttggatgctgatgtcgcaaggaaccac

SEQ ID NO: 114 - DNA, 30 bp, Artificial Sequence
  Primer
acgttggatgcctgttgaattatggaggag

SEQ ID NO: 115 - DNA, 31 bp, Artificial Sequence
  Primer
acgttggatgctcttctttccatggatcttc

SEQ ID NO: 116 - DNA, 31 bp, Artificial Sequence; identical to SEQ ID NO: 115
  Primer

SEQ ID NO: 117 - DNA, 30 bp, Artificial Sequence
  Primer
acgttggatgcagctaatttctcctgacag

SEQ ID NO: 118 - DNA, 30 bp, Artificial Sequence
  Primer
acgttggatgatgcaaactggctgggaatg

SEQ ID NO: 119 - DNA, 30 bp, Artificial Sequence
  Primer
acgttggatggaggaggctgtgagaaaaga

SEQ ID NO: 120 - DNA, 30 bp, Artificial Sequence
  Primer
acgttggatgatgccctggattgacctaac

SEQ ID NO: 121 - DNA, 30 bp, Artificial Sequence
  Primer
acgttggatggggttagggtgtgtataagg

SEQ ID NO: 122 - DNA, 30 bp, Artificial Sequence
  Primer
acgttggatgaacccgcactacaagattcc

SEQ ID NO: 123 - DNA, 30 bp, Artificial Sequence
  Primer
acgttggatggtcagtcccacattcagaac

SEQ ID NO: 124 - DNA, 30 bp, Artificial Sequence
  Primer
acgttggatgtcccgaacataaagactcag

SEQ ID NO: 125 - DNA, 30 bp, Artificial Sequence
  Primer
acgttggatgggttgtaattggaacattgg

SEQ ID NO: 126 - DNA, 30 bp, Artificial Sequence
  Primer
acgttggatggtctgccaaagttcccttag

SEQ ID NO: 127 - DNA, 30 bp, Artificial Sequence
  Primer
acgttggatgaggaaagggaagagaaaccg

SEQ ID NO: 128 - DNA, 30 bp, Artificial Sequence
  Primer
acgttggatggactgtgagttataggatac

SEQ ID NO: 129 - DNA, 30 bp, Artificial Sequence
  Primer
acgttggatgatgggtcggaggatttatag

SEQ ID NO: 130 - DNA, 30 bp, Artificial Sequence
  Primer
acgttggatgctcggctaaggtactcaata

SEQ ID NO: 131 - DNA, 30 bp, Artificial Sequence
  Primer
acgttggatgagaccaccaagtaaaattgc

SEQ ID NO: 132 - DNA, 30 bp, Artificial Sequence
  Primer
acgttggatgttgggttaatgcagggtctg

SEQ ID NO: 133 - DNA, 30 bp, Artificial Sequence
  Primer
acgttggatgctagttcacctgggtctatc

SEQ ID NO: 134 - DNA, 31 bp, Artificial Sequence
  Primer
acgttggatgccaaagcccatgttttaaaaa

SEQ ID NO: 135 - DNA, 30 bp, Artificial Sequence
  Primer
acgttggatggtttttctaaaatatgggct

SEQ ID NO: 136 - DNA, 30 bp, Artificial Sequence
  Primer
acgttggatgaaaccagctcaggccattac

SEQ ID NO: 137 - DNA, 30 bp, Artificial Sequence
  Primer
acgttggatgatgcaaaataagctctgccc

SEQ ID NO: 138 - DNA, 30 bp, Artificial Sequence
  Primer
acgttggatgggtgtactctgccatttgtc

SEQ ID NO: 139 - DNA, 30 bp, Artificial Sequence
  Primer
acgttggatgtggaggaggttctagtaccc

SEQ ID NO: 140 - DNA, 30 bp, Artificial Sequence
  Primer
acgttggatgagttgtgagtgatttcaggg

SEQ ID NO: 141 - DNA, 30 bp, Artificial Sequence
  Primer
acgttggatgcaggccttcttgctcttatc

SEQ ID NO: 142 - DNA, 30 bp, Artificial Sequence
  Primer
acgttggatgctgtgccttctgagtagttc

SEQ ID NO: 143 - DNA, 30 bp, Artificial Sequence
  Primer
acgttggatgatctgttgccttaggttcac

SEQ ID NO: 144 - DNA, 30 bp, Artificial Sequence
  Primer
acgttggatgctctcaattccatcagtctc

SEQ ID NO: 145 - DNA, 31 bp, Artificial Sequence
  Primer
acgttggatgctttacgaatttcacaacagg

SEQ ID NO: 146 - DNA, 30 bp, Artificial Sequence
  Primer
acgttggatgtatgcttccagtctctgacc

SEQ ID NO: 147 - DNA, 30 bp, Artificial Sequence
  Primer
acgttggatgataggtaatccagttgggcc

SEQ ID NO: 148 - DNA, 30 bp, Artificial Sequence
  Primer
acgttggatgctcaggtaatctgccttctc

SEQ ID NO: 149 - DNA, 31 bp, Artificial Sequence
  Primer
acgttggatgcagggataatgagaacaaatc

SEQ ID NO: 150 - DNA, 30 bp, Artificial Sequence
  Primer
acgttggatgaacaaccttacttcatgccc

SEQ ID NO: 151 - DNA, 30 bp, Artificial Sequence
  Primer
acgttggatgttctccactttctggtcaac

SEQ ID NO: 152 - DNA, 30 bp, Artificial Sequence
  Primer
acgttggatgtggatactgagggtcaactg

SEQ ID NO: 153 - DNA, 30 bp, Artificial Sequence
  Primer
acgttggatgcttcccaacattttcggctc

SEQ ID NO: 154 - DNA, 18 bp, Artificial Sequence
  Primer
gtcccctttcatctaaac

SEQ ID NO: 155 - DNA, 17 bp, Artificial Sequence
  Primer
tctgcagcgtgcggcga

SEQ ID NO: 156 - DNA, 19 bp, Artificial Sequence
  Primer
ccagggtatgagcggagga

SEQ ID NO: 157 - DNA, 22 bp, Artificial Sequence
  Primer
agtgcacacagaacatttaaca

SEQ ID NO: 158 - DNA, 20 bp, Artificial Sequence
  Primer
tgtggagacaaggtctcact

SEQ ID NO: 159 - DNA, 17 bp, Artificial Sequence
  Primer
tgggcaaacaagcccat

SEQ ID NO: 160 - DNA, 17 bp, Artificial Sequence
  Primer
tggtcatacccttcaag

SEQ ID NO: 161 - DNA, 17 bp, Artificial Sequence
  Primer
gaagggtatgaccaagc

SEQ ID NO: 162 - DNA, 17 bp, Artificial Sequence
  Primer
agttgttcagatcctcc

SEQ ID NO: 163 - DNA, 22 bp, Artificial Sequence
  Primer
tccaaaaccaccagataaaatc

SEQ ID NO: 164 - DNA, 17 bp, Artificial Sequence
  Primer
gtattgtcctccagtga

SEQ ID NO: 165 - DNA, 21 bp, Artificial Sequence
  Primer
agaatggtggtgtatttttac

SEQ ID NO: 166 - DNA, 20 bp, Artificial Sequence
  Primer
tagtcctacacaatctgtta

SEQ ID NO: 167 - DNA, 17 bp, Artificial Sequence
  Primer
ggtatcaggaagagtca

SEQ ID NO: 168 - DNA, 21 bp, Artificial Sequence
  Primer
ccctgacaattccaaaactaa

SEQ ID NO: 169 - DNA, 18 bp, Artificial Sequence
  Primer
ggaggaggcactattaat

SEQ ID NO: 170 - DNA, 17 bp, Artificial Sequence
  Primer
ggccaccttcatatttc

SEQ ID NO: 171 - DNA, 19 bp, Artificial Sequence
  Primer
caggagatccagaccatcc

SEQ ID NO: 172 - DNA, 18 bp, Artificial Sequence
  Primer
tgcggcccccgccgggtt

SEQ ID NO: 173 - DNA, 19 bp, Artificial Sequence
  Primer
gaattgcccggctccgaat

SEQ ID NO: 174 - DNA, 17 bp, Artificial Sequence
  Primer
gagcaggcagcacaagt

SEQ ID NO: 175 - DNA, 23 bp, Artificial Sequence
  Primer
acctccaggtaaaatgattagtt

SEQ ID NO: 176 - DNA, 17 bp, Artificial Sequence
  Primer
cagggatggtaattgac

SEQ ID NO: 177 - DNA, 20 bp, Artificial Sequence
  Primer
aagcggaggttgcagtgagc

SEQ ID NO: 178 - DNA, 19 bp, Artificial Sequence
  Primer
gacatgtccctctcggcct

SEQ ID NO: 179 - DNA, 17 bp, Artificial Sequence
  Primer
ggcaatgtttgcccttt

SEQ ID NO: 180 - DNA, 19 bp, Artificial Sequence
  Primer
gggagaaagtaacagggtc

SEQ ID NO: 181 - DNA, 17 bp, Artificial Sequence
  Primer
caggtggatggggacac

SEQ ID NO: 182 - DNA, 17 bp, Artificial Sequence
  Primer
tggtgttaagtggcgtg

SEQ ID NO: 183 - DNA, 19 bp, Artificial Sequence
  Primer
cacaccaccacgcccggct

SEQ ID NO: 184 - DNA, 20 bp, Artificial Sequence
  Primer
ctgctattcatttgtgtaga

SEQ ID NO: 185 - DNA, 19 bp, Artificial Sequence
  Primer
ctctgatgttacctcctcc

SEQ ID NO: 186 - DNA, 20 bp, Artificial Sequence
  Primer
ctaggctagcagtggggttg

SEQ ID NO: 187 - DNA, 18 bp, Artificial Sequence
  Primer
ggcagggaagagaagagc

SEQ ID NO: 188 - DNA, 21 bp, Artificial Sequence
  Primer
agatgccaagaccattcaaag

SEQ ID NO: 189 - DNA, 19 bp, Artificial Sequence
  Primer
tagttgatgaagatttggg

SEQ ID NO: 190 - DNA, 20 bp, Artificial Sequence
  Primer
aggttacccctgctgacttt

SEQ ID NO: 191 - DNA, 20 bp, Artificial Sequence
  Primer
gagatcatttctccttcaac

SEQ ID NO: 192 - DNA, 23 bp, Artificial Sequence
  Primer
ccacacccatatatatttatgct

SEQ ID NO: 193 - DNA, 21 bp, Artificial Sequence
  Primer
aaagatacaccgttgagaagg

SEQ ID NO: 194 - DNA, 20 bp, Artificial Sequence
  Primer
gactccctttaccttcatgg

SEQ ID NO: 195 - DNA, 19 bp, Artificial Sequence
  Primer
cttcactgagcgtggtgcc

SEQ ID NO: 196 - DNA, 19 bp, Artificial Sequence
  Primer
cacagatgctcatgggtcc

SEQ ID NO: 197 - DNA, 24 bp, Artificial Sequence
  Primer
gtttgcccaacatataaacaataa

SEQ ID NO: 198 - DNA, 23 bp, Artificial Sequence
  Primer
aacacagtcaaaattttgcttca

SEQ ID NO: 199 - DNA, 23 bp, Artificial Sequence
  Primer
gagccaagtttacatcaagttta

SEQ ID NO: 200 - DNA, 21 bp, Artificial Sequence
  Primer
agatcacattgccaaccccca

SEQ ID NO: 201 - DNA, 18 bp, Artificial Sequence
  Primer
cccgtctcctgctggtca

SEQ ID NO: 202 - DNA, 22 bp, Artificial Sequence
  Primer
ccctaaagcatctcacagcccc

SEQ ID NO: 203 - DNA, 19 bp, Artificial Sequence
  Primer
cacatgcctgctcgccccc

SEQ ID NO: 204 - DNA, 18 bp, Artificial Sequence
  Primer
cacatgcctgctcgcccc

SEQ ID NO: 205 - DNA, 19 bp, Artificial Sequence
  Primer
tgcctttggacgtctagcc

SEQ ID NO: 206 - DNA, 22 bp, Artificial Sequence
  Primer
tatggaggagtagatattggaa

SEQ ID NO: 207 - DNA, 23 bp, Artificial Sequence
  Primer
gaaaattccaatatctactcctc

SEQ ID NO: 208 - DNA, 21 bp, Artificial Sequence
  Primer
ctgggaatgaaattagggcag

SEQ ID NO: 209 - DNA, 20 bp, Artificial Sequence
  Primer
gttcccttgactttcctcag

SEQ ID NO: 210 - DNA, 18 bp, Artificial Sequence
  Primer
gcatcttcaggaatcttg

SEQ ID NO: 211 - DNA, 21 bp, Artificial Sequence
  Primer
cataaagactcagcattcagc

SEQ ID NO: 212 - DNA, 19 bp, Artificial Sequence
  Primer
ggttaggaagaaatctgtg

SEQ ID NO: 213 - DNA, 23 bp, Artificial Sequence
  Primer
atctagataataaagaccaccaa

SEQ ID NO: 214 - DNA, 23 bp, Artificial Sequence
  Primer
ctattaatggtgtttgtctatgg

SEQ ID NO: 215 - DNA, 20 bp, Artificial Sequence
  Primer
taatgcagggtctgctggat

SEQ ID NO: 216 - DNA, 23 bp, Artificial Sequence
  Primer
atctctaagatataacactctac

SEQ ID NO: 217 - DNA, 20 bp, Artificial Sequence
  Primer
gtgcctgcaaagaaaggaac

SEQ ID NO: 218 - DNA, 20 bp, Artificial Sequence
  Primer
ttgtcagttatgagaccttg

SEQ ID NO: 219 - DNA, 24 bp, Artificial Sequence
  Primer
atacctcagaatacagcttttttt

SEQ ID NO: 220 - DNA, 20 bp, Artificial Sequence
  Primer
tctcatttctcctctctttc

SEQ ID NO: 221 - DNA, 17 bp, Artificial Sequence
  Primer
ctcatttccacccttct

SEQ ID NO: 222 - DNA, 20 bp, Artificial Sequence
  Primer
gtctctgaccacttgaccca

SEQ ID NO: 223 - DNA, 17 bp, Artificial Sequence
  Primer
tccttcttctctgcttt

SEQ ID NO: 224 - DNA, 18 bp, Artificial Sequence
  Primer
tcatgcccattgggttag

SEQ ID NO: 225 - DNA, 17 bp, Artificial Sequence
  Primer
gggtcaactgtaccaag

SEQ ID NO: 226 - DNA, 30 bp, Artificial Sequence
  Primer
acgttggatgtgtcagaaagcaaacctggc

SEQ ID NO: 227 - DNA, 30 bp, Artificial Sequence
  Primer
acgttggatgttacaggcattggagacagc

SEQ ID NO: 228 - DNA, 30 bp, Artificial Sequence
  Primer
acgttggatgctgcataatggtggctttgg

SEQ ID NO: 229 - DNA, 30 bp, Artificial Sequence
  Primer
acgttggatgtgtgggtgttcactttgcag

SEQ ID NO: 230 - DNA, 30 bp, Artificial Sequence
  Primer
acgttggatgccctcttgtcaggaagttct

SEQ ID NO: 231 - DNA, 30 bp, Artificial Sequence
  Primer
acgttggatggagacagagttgaactcccg

SEQ ID NO: 232 - DNA, 30 bp, Artificial Sequence; identical to SEQ ID NO: 109
  Primer

SEQ ID NO: 233 - DNA, 30 bp, Artificial Sequence; identical to SEQ ID NO: 108
  Primer

SEQ ID NO: 234 - DNA, 30 bp, Artificial Sequence; identical to SEQ ID NO: 91
  Primer

SEQ ID NO: 235 - DNA, 30 bp, Artificial Sequence; identical to SEQ ID NO: 90
  Primer

SEQ ID NO: 236 - DNA, 30 bp, Artificial Sequence
  Primer
acgttggatgcgcactacaagattccaagc

SEQ ID NO: 237 - DNA, 30 bp, Artificial Sequence
  Primer
acgttggatgtcagtcccacattcagaacc

SEQ ID NO: 238 - DNA, 30 bp, Artificial Sequence; identical to SEQ ID NO: 229
  Primer

SEQ ID NO: 239 - DNA, 30 bp, Artificial Sequence; identical to SEQ ID NO: 228
  Primer

SEQ ID NO: 240 - DNA, 30 bp, Artificial Sequence; identical to SEQ ID NO: 12
  Primer

SEQ ID NO: 241 - DNA, 30 bp, Artificial Sequence; identical to SEQ ID NO: 13
  Primer

SEQ ID NO: 242 - DNA, 30 bp, Artificial Sequence
  Primer
acgttggatggatgcactgaagggagaaag

SEQ ID NO: 243 - DNA, 30 bp, Artificial Sequence; identical to SEQ ID NO: 63
  Primer

SEQ ID NO: 244 - DNA, 30 bp, Artificial Sequence; identical to SEQ ID NO: 25
  Primer

SEQ ID NO: 245 - DNA, 30 bp, Artificial Sequence; identical to SEQ ID NO: 24
  Primer

SEQ ID NO: 246 - DNA, 30 bp, Artificial Sequence; identical to SEQ ID NO: 141
  Primer

SEQ ID NO: 247 - DNA, 30 bp, Artificial Sequence; identical to SEQ ID NO: 140
  Primer

SEQ ID NO: 248 - DNA, 30 bp, Artificial Sequence; identical to SEQ ID NO: 146
  Primer

SEQ ID NO: 249 - DNA, 30 bp, Artificial Sequence
  Primer
acgttggatggataggtaatccagttgggc

SEQ ID NO: 250 - DNA, 30 bp, Artificial Sequence; identical to SEQ ID NO: 105
  Primer

SEQ ID NO: 251 - DNA, 30 bp, Artificial Sequence; identical to SEQ ID NO: 104
  Primer

SEQ ID NO: 252 - DNA, 30 bp, Artificial Sequence; identical to SEQ ID NO: 74
  Primer

SEQ ID NO: 253 - DNA, 30 bp, Artificial Sequence; identical to SEQ ID NO: 75
  Primer

SEQ ID NO: 254 - DNA, 17 bp, Artificial Sequence
  Primer
tgatgcacgtggagcag

SEQ ID NO: 255 - DNA, 17 bp, Artificial Sequence
  Primer
gcccctggaaaaggccc

SEQ ID NO: 256 - DNA, 20 bp, Artificial Sequence
  Primer
ggaagatgatgagactaaat

SEQ ID NO: 257 - DNA, 19 bp, Artificial Sequence
  Primer
cacatgcctgctcgccccc

SEQ ID NO: 258 - DNA, 17 bp, Artificial Sequence
  Primer
tccctttaccttcatgg

SEQ ID NO: 259 - DNA, 18 bp, Artificial Sequence
  Primer
gcatcttcaggaatcttg

SEQ ID NO: 260 - DNA, 17 bp, Artificial Sequence
  Primer
gcccctggaaaaggccc

SEQ ID NO: 261 - DNA, 20 bp, Artificial Sequence
  Primer
ggttctgcagcgtgcggcga

SEQ ID NO: 262 - DNA, 22 bp, Artificial Sequence
  Primer
gaagggagaaagtaacagggtc

SEQ ID NO: 263 - DNA, 17 bp, Artificial Sequence
  Primer
tggtcatacccttcaag

SEQ ID NO: 264 - DNA, 21 bp, Artificial Sequence
  Primer
atcttctacacattgattcag

SEQ ID NO: 265 - DNA, 19 bp, Artificial Sequence
  Primer
tctctgaccacttgaccca

SEQ ID NO: 266 - DNA, 17 bp, Artificial Sequence
  Primer
tggtgaccagcaggaga

SEQ ID NO: 267 - DNA, 17 bp, Artificial Sequence
  Primer
ggctagcagtggggttg

SEQ ID NO: 268 - DNA, 20 bp, Artificial Sequence
  Primer
ccagtcgaaatggacttgag

SEQ ID NO: 269 - DNA, 20 bp, Artificial Sequence
  Primer
cgccttcacagtcttcaaag

SEQ ID NO: 270 - DNA, 19 bp, Artificial Sequence
  Primer
gagacaagtggagctccgt

SEQ ID NO: 271 - DNA, 19 bp, Artificial Sequence
  Primer
gtggagctccgtaaaggca

SEQ ID NO: 272 - DNA, 19 bp, Artificial Sequence
  Primer
atcaccgcactgccatcga

SEQ ID NO: 273 - DNA, 19 bp, Artificial Sequence
  Primer
gcatgctatccacggaaga

SEQ ID NO: 274 - DNA, 19 bp, Artificial Sequence
  Primer
gagacaagtggagctccgt

SEQ ID NO: 275 - DNA, 19 bp, Artificial Sequence
  Primer
cgtacgcggaatacttcga

(19) United States
(12) Patent Application Publication
(10) Pub. No.: US 2009/0258344 A1
(43) Pub. Date: Oct. 15, 2009

(54) METHODS FOR IDENTIFYING RISK OF BREAST CANCER AND TREATMENTS THEREOF

(75) Inventors: Richard B. Roth, San Diego, CA (US); Matthew Roberts Nelson, San Marcos, CA (US); Andreas Braun, San Diego, CA (US); Stefan M. Kammerer, San Diego, CA (US); Mikhail F. Denissenko, Poway, CA (US); Rikard Reneland, Knivsta (SE); Caridad Rosette, San Diego, CA (US); Carolyn R. Hoyal-Wrightson, San Diego, CA (US)

Correspondence Address: GRANT ANDERSON LLP;C/O PORTFOLIOIP, PO BOX 52050, MINNEAPOLIS, MN, 55402, US

(73) Assignee: SEQUENOM, INC., San Diego, CA (US)

(21) Appl. No.: 11/570,427

(22) PCT Filed: May 27, 2004
(86) PCT No.: PCT/US04/16942

§ 371 Date: Dec. 12, 2008

(51) Int. Cl. C12Q 1/68; C07H 21/00; C40B 40/06; C07K 2/00

(52) U.S. Cl. 435/6; 536/23.1; 506/16; 530/300

(57) ABSTRACT

Provided herein are methods for identifying risk of breast cancer in a subject and/or a subject at risk of breast cancer, reagents and kits for carrying out the methods, methods for identifying candidate therapeutics for treating breast cancer, and therapeutic methods for treating breast cancer in a subject. These embodiments are based upon an analysis of polymorphic variations in nucleotide sequences within the human genome.

### RELATED PATENT APPLICATIONS

This patent application claims priority to international application no. PCT/US2004/016942 filed May 27, 2004, having attorney docket number SEQ-4066-PC2. This patent application names Richard B. Roth et al. as inventors and is hereby incorporated herein by reference in its entirety, including all drawings and cited publications and documents.

### FIELD OF THE INVENTION

The invention relates to genetic methods for identifying risk of breast cancer and treatments that specifically target the disease.

### BACKGROUND

Breast cancer is the third most common cancer, and the most common cancer in women, as well as a cause of disability, psychological trauma, and economic loss. Breast cancer is the second most common cause of cancer death in women in the United States, in particular for women between the ages of 15 and 54, and the leading cause of cancer-related death (Forbes, Seminars in Oncology, vol. 24(1), Suppl 1, 1997: pp. S1-20-S1-35). Indirect effects of the disease also contribute to the mortality from breast cancer including consequences of advanced disease, such as metastases to the bone or brain. Complications arising from bone marrow suppression, radiation fibrosis and neutropenic sepsis, collateral effects from therapeutic interventions, such as surgery, radiation, chemotherapy, or bone marrow transplantation—also contribute to the morbidity and mortality from this disease.

While the pathogenesis of breast cancer is unclear, transformation of normal breast epithelium to a malignant phenotype may be the result of genetic factors, especially in women under thirty (Miki, et al., Science, 266: 66-71 (1994)). However, it is likely that other, non-genetic factors also have a significant effect on the etiology of the disease. Regardless of its origin, breast cancer morbidity increases significantly if it is not detected early in its progression. Thus, considerable efforts have focused on the elucidation of early cellular events surrounding transformation in breast tissue. Such efforts have led to the identification of several potential breast cancer markers. For example, alleles of the BRCA1 and BRCA2 genes have been linked to hereditary and early-onset breast cancer (Wooster, et al., Science, 265: 2088-2090 (1994)). However, BRCA1 is limited as a cancer marker because BRCA1 mutations fail to account for the majority of breast cancers (Ford, et al., British J. Cancer, 72: 805-812 (1995)). Similarly, the BRCA2 gene, which has been linked to forms of hereditary breast cancer, accounts for only a small portion of total breast cancer cases.

### SUMMARY

It has been discovered that certain polymorphic variations in human genomic DNA are associated with the occurrence of breast cancer. Thus, featured herein are methods for identifying a subject at risk of breast cancer and/or a risk of breast cancer in a subject, which comprises detecting the presence or absence of one or more of the polymorphic variations described herein in a human nucleic acid sample. Also featured herein are nucleic acids that include one or more polymorphic variations associated with the occurrence of breast cancer, as well as polypeptides encoded by these nucleic acids. Further, provided is a method for identifying a subject at risk of breast cancer and then prescribing to the subject a breast cancer detection procedure, prevention procedure and/or a treatment procedure. In addition, provided are methods for identifying candidate therapeutic molecules for treating breast cancer and related disorders, as well as methods for treating breast cancer in a subject by diagnosing breast cancer in the subject and treating the subject with a suitable treatment, such as administering a therapeutic molecule.

Also, featured is a method for inhibiting metastasis of breast cancer cells into other tissues, which comprises inhibiting a KIAA0861 nucleic acid or substantially identical nucleic acid thereof (e.g., reducing the amount of polypeptide expressed from mRNA encoded by the nucleotide sequence), or inhibiting a KIAA0861 polypeptide or substantially identical polypeptide thereof (e.g., inhibiting the guanine nucleotide exchange function of the KIAA0861 polypeptide). The inhibition is effected by contacting a system with a molecule having the inhibitory activity, where the system sometimes is a group of cells in vitro, a tissue sample in vitro, or an animal such as a human, often a female. In an embodiment, the KIAA0861 nucleic acid or substantially identical nucleic acid thereof is inhibited by contacting cells overexpressing the KIAA0861 nucleotide sequence with an RNA molecule, and in certain embodiments, the RNA molecule is double stranded with one strand complementary to a subsequence of the KIAA0861 nucleotide sequence.

Also provided are compositions comprising a breast cancer cell and/or a KIAA0861 nucleic acid with a RNAi, siRNA, antisense DNA or RNA, or ribozyme nucleic acid designed from a KIAA0861 nucleotide sequence. In an embodiment, the nucleic acid is designed from a KIAA0861 nucleotide sequence that includes one or more breast cancer associated polymorphic variations, and in some instances, specifically interacts with such a nucleotide sequence. Further, provided are arrays of nucleic acids bound to a solid surface, in which one or more nucleic acid molecules of the array have a KIAA0861 nucleotide sequence, or a fragment or substantially identical nucleic acid thereof, or a complementary nucleic acid of the foregoing. Featured also are compositions comprising a breast cancer cell and/or a KIAA0861 polypeptide, with an antibody that specifically binds to the polypeptide. In an embodiment, the antibody specifically binds to an epitope in the polypeptide that includes a non-synonymous amino acid modification associated with breast cancer (e.g., results in an amino acid substitution in the encoded polypeptide associated with breast cancer). In certain embodiments, the antibody specifically binds to an epitope that comprises a leucine at amino acid position 276 in SEQ ID NO: 4, a leucine at amino acid position 295 in SEQ ID NO: 4, a phenylalanine at amino acid position 506 in SEQ ID NO: 4, or an alanine at amino acid position 819 in SEQ ID NO: 4. Alternatively, the antibody specifically binds to an epitope that comprises a leucine at amino acid position 359 in SEQ ID NO: 5, a leucine at amino acid position 378 in SEQ ID NO: 5, a phenylalanine at amino acid position 589 in SEQ ID NO: 4, or an alanine at amino acid position 902 in SEQ ID NO: 5.

### BRIEF DESCRIPTION OF THE FIGURES

FIG. 1 shows proximal SNPs in and around a KIAA0861 region. The position of each SNP on the chromosome is shown on the x-axis and the y-axis provides the negative logarithm of the p-value comparing the estimated allele to that of the control group. Also shown in the figure are exons and introns of the region in the approximate chromosomal positions. The figure indicates that polymorphic variants associated with breast cancer are in a region spanning chromosome positions 184215647 to 184249849 on chromosome 3 (based on NCBI's Build 34).

### DETAILED DESCRIPTION

It has been discovered that a polymorphic variation in a gene encoding a novel member of the DBL family of Rho guanine nucleotide exchange factors (RhoGEFs), known as KIAA0861, is associated with the occurrence of breast cancer. DBL RhoGEF proteins are characterized by two distinct domains, the Dbl homology (DH) domain and the pleckstrin homology (PH) domain, which are believed to be responsible for catalyzing the GDP-GTP exchange reaction of Rho proteins. RhoGEFs bind to the GDP-bound form and destabilize GDP-RhoGTPases while stabilizing a nucleotide-free reaction intermediate. Because of the high intracellular ratio of GTP:GDP, the released GTP is replaced with GTP, leading to activation. Rho family GTP-binding proteins (Rho GTPases) belong to the Ras-related G protein superfamily and function in controlling numerous cellular activities, including cell growth, adhesion, and movement. The Rho GTPase family includes members RhoA, RacI and Cdc42, which stimulate the cyclin D1 promoter and cause upregulation of cyclin D1 protein. RacI and Cdc42 promote inactivation of Rb and stimulation of E2F-mediated transcription. Signaling pathways for RhoGEFs and RhoGTPases are set forth in Schmidt & Hall, Genes and Development 16: 1587-1609 (2002) and Pruitt & Der, Cancer Letters 171: 1-10 (2001).

KIAA0861 shares strong homology with members of the Dbl family of Rho guanine nucleotide exchange factors (RhoGEFs), a family of over 60 proteins that function by catalyzing the exchange of Rho-bound GDP for GTP. Rho family GTP-binding proteins (Rho GTPases) belong to the Ras-related G protein superfamily and function in controlling numerous cellular activities, including cell growth, adhesion and movement. Over 18 Ras-related G protein superfamily members have been described to date, including several Rho-family GTP-binding proteins (Rho GTPases). Rho GTPases are membrane bound molecular switches that are active when bound to GTP and inactive when bound to GDP. Deregulation of both Rho GTPase activity and RhoGEF activity have been shown to be oncogenic. Several studies have shown that deregulation of Rho GTPase activity leads to loss of contact inhibition, growth factor dependence and anchorage dependence in a variety of cell types (Whitehead, I P, et al (1997) Biochem. Biophys. Acta, 1332: F1-F23).

Deregulation of both Rho GTPase activity and RhoGEF activity have been shown to be oncogenic. For example, DBS, a Rho-specific guanine nucleotide exchange factor (RhoGEF), exhibits transforming activity when overexpressed in NIH 3T3 mouse fibroblasts (Cheng, L, et al. (2002) MCB 22 (19):6895-6905). Several studies have shown that deregulation of Rho GTPase activity leads to loss of contact inhibition, growth factor dependence and anchorage dependence in a variety of cell types. Further, recent evidence has shown that deregulation of RhoGEF activity results in tumorigenic growth and promotes invasive potential (Whitehead, I P, et al. (1997) Biochem. Biophys. Acta, 1332: F1-F23). Interestingly, cellular transformation via deregulated RhoGEF function is much stronger than through deregulation of Rho GTPases (Lin, R, Cerione, R A, and Manor, D (1999) JBC, 274: 23633-23641).

### Breast Cancer and Sample Selection

Breast cancer is typically described as the uncontrolled growth of malignant breast tissue. Breast cancers arise most commonly in the lining of the milk ducts of the breast (ductal carcinoma), or in the lobules where breast milk is produced (lobular carcinoma). Other forms of breast cancer include Inflammatory Breast Cancer and Recurrent Breast Cancer. Inflammatory breast cancer is a rare, but very serious, aggressive type of breast cancer. The breast may look red and feel warm with ridges, welts, or hives on the breast; or the skin may look wrinkled. It is sometimes misdiagnosed as a simple infection. Recurrent disease means that the cancer has come back after it has been treated. It may come back in the breast, in the soft tissues of the chest (the chest wall), or in another part of the body.

As used herein, the term “breast cancer” refers to a condition characterized by anomalous rapid proliferation of abnormal cells in one or both breasts of a subject. The abnormal cells often are referred to as “neoplastic cells,” which are transformed cells that can form a solid tumor. The term “tumor” refers to an abnormal mass or population of cells (i.e. two or more cells) that result from excessive or abnormal cell division, whether malignant or benign, and pre-cancerous and cancerous cells. Malignant tumors are distinguished from benign growths or tumors in that, in addition to uncontrolled cellular proliferation, they can invade surrounding tissues and can metastasize. In breast cancer, neoplastic cells may be identified in one or both breasts only and not in another tissue or organ, in one or both breasts and one or more adjacent tissues or organs (e.g. lymph node), or in a breast and one or more non-adjacent tissues or organs to which the breast cancer cells have metastasized.

The term “invasion” as used herein refers to the spread of cancerous cells to adjacent surrounding tissues. The term “invasion” often is used synonymously with the term “metastasis,” which as used herein refers to a process in which cancer cells travel from one organ or tissue to another non-adjacent organ or tissue. Cancer cells in the breast(s) can spread to tissues and organs of a subject, and conversely, cancer cells from other organs or tissue can invade or metastasize to a breast. Cancerous cells from the breast(s) may invade or metastasize to any other organ or tissue of the body. Breast cancer cells often invade lymph node cells and/or metastasize to the liver, brain and/or bone and spread cancer in these tissues and organs. Breast cancers can spread to other organs and tissues and cause lung cancer, prostate cancer, colon cancer, ovarian cancer, cervical cancer, gastrointestinal cancer, pancreatic cancer, glioblastoma, bladder cancer, hepatoma, colorectal cancer, uterine cervical cancer, endometrial carcinoma, salivary gland carcinoma, kidney cancer, vulval cancer, thyroid cancer, hepatic carcinoma, skin cancer, melanoma, ovarian cancer, neuroblastoma, myeloma, various types of head and neck cancer, acute lymphoblastic leukemia, acute myeloid leukemia, Ewing sarcoma and peripheral neuroepithelioma, and other carcinomas, lymphomas, blastomas, sarcomas, and leukemias.

Breast cancers arise most commonly in the lining of the milk ducts of the breast (ductal carcinoma), or in the lobules where breast milk is produced (lobular carcinoma). Other forms of breast cancer include Inflammatory Breast Cancer and Recurrent Breast Cancer. Inflammatory Breast Cancer is a rare, but very serious, aggressive type of breast cancer. The breast may look red and feel warm with ridges, welts, or hives on the breast; or the skin may look wrinkled. It is sometimes misdiagnosed as a simple infection. Recurrent disease means that the cancer has come back after it has been treated. It may come back in the breast, in the soft tissues of the chest (the chest wall), or in another part of the body. As used herein, the term “breast cancer” may include both Inflammatory Breast Cancer and Recurrent Breast Cancer.

In an effort to detect breast cancer as early as possible, regular physical exams and screening mammograms often are prescribed and conducted. A diagnostic mammogram often is performed to evaluate a breast complaint or abnormality detected by physical exam or routine screening mammography. If an abnormality seen with diagnostic mammography is suspicious, additional breast imaging (with exams such as ultrasound) or a biopsy may be ordered. A biopsy followed by pathological (microscopic) analysis is a definitive way to determine whether a subject has breast cancer. Excised breast cancer samples often are subjected to the following analyses: diagnosis of the breast tumor and confirmation of its malignancy; maximum tumor thickness; assessment of completeness of excision of invasive and in situ components and microscopic measurements of the shortest extent of clearance; level of invasion; presence and extent of regression; presence and extent of ulceration; histological type and special variants; pre-existing lesion; mitotic rate; vascular invasion; neurotropism; cell type; tumor lymphocyte infiltration; and growth phase.

The stage of a breast cancer can be classified as a range of stages from Stage 0 to Stage IV based on its size and the extent to which it has spread. The following table summarizes the stages:

TABLE A

| | | | Metastasis |
|---|---|---|---|
| Stage | Tumor Size | Lymph Node Involvement | (Spread) |
| I | Less than 2 cm | No | No |
| II | Between 2-5 cm | No or in same side of breast | No |
| III | More than 5 cm | Yes, on same side of breast | No |
| IV | Not applicable | Not applicable | Yes |

Stage 0 cancer is a contained cancer that has not spread beyond the breast ductal system. Fifteen to twenty percent of breast cancers detected by clinical examinations or testing are in Stage 0 (the earliest form of breast cancer). Two types of Stage 0 cancer are lobular carcinoma in situ (LCIS) and ductal carcinoma in situ (DCIS). LCIS indicates high risk for breast cancer. Many physicians do not classify LCIS as a malignancy and often encounter LCIS by chance on breast biopsy while investigating another area of concern. While the microscopic features of LCIS are abnormal and are similar to malignancy, LCIS does not behave as a cancer (and therefore is not treated as a cancer). LCIS is merely a marker for a significantly increased risk of cancer anywhere in the breast. However, bilateral simple mastectomy may be occasionally performed if LCIS patients have a strong family history of breast cancer. In DCIS the cancer cells are confined to milk ducts in the breast and have not spread into the fatty breast tissue or to any other part of the body (such as the lymph nodes). DCIS may be detected on mammogram as tiny specks of calcium (known as microcalcifications) 80% of the time. Less commonly DCIS can present itself as a mass with calcifications (15% of the time); and even less likely as a mass without calcifications (<5% of the time). A breast biopsy is used to confirm DCIS. A standard DCIS treatment is breast-conserving therapy (BCT), which is lumpectomy followed by radiation treatment or mastectomy. To date, DCIS patients have chosen equally among lumpectomy and mastectomy as their treatment option, though specific cases may sometimes favor lumpectomy over mastectomy or vice versa.

In Stage I, the primary (original) cancer is 2 cm or less in diameter and has not spread to the lymph nodes. In Stage IIA, the primary tumor is between 2 and 5 cm in diameter and has not spread to the lymph nodes. In Stage IIB, the primary tumor is between 2 and 5 cm in diameter and has spread to the axillary (underarm) lymph nodes; or the primary tumor is over 5 cm and has not spread to the lymph nodes. In Stage IIIA, the primary breast cancer of any kind that has spread to the axillary (underarm) lymph nodes and to axillary tissues. In Stage IIIB, the primary breast cancer is any size, has attached itself to the chest wall, and has spread to the pectoral (chest) lymph nodes. In Stage IV, the primary cancer has spread out of the breast to other parts of the body (such as bone, lung, liver, brain). The treatment of Stage IV breast cancer focuses on extending survival time and relieving symptoms.

Based in part upon selection criteria set forth above, individuals having breast cancer can be selected for genetic studies. Also, individuals having no history of cancer or breast cancer often are selected for genetic studies. Other selection criteria can include: a tissue or fluid sample is derived from an individual characterized as Caucasian; the sample was derived from an individual of German paternal and maternal descent; the database included relevant phenotype information for the individual; case samples were derived from individuals diagnosed with breast cancer; control samples were derived from individuals free of cancer and no family history of breast cancer; and sufficient genomic DNA was extracted from each blood sample for all allelotyping and genotyping reactions performed during the study. Phenotype information included pre- or post-menopausal, familial predisposition, country or origin of mother and father, diagnosis with breast cancer (date of primary diagnosis, age of individual as of primary diagnosis, grade or stage of development, occurrence of metastases, e.g., lymph node metastases, organ metastases), condition of body tissue (skin tissue, breast tissue, ovary tissue, peritoneum tissue and myometrium), method of treatment (surgery, chemotherapy, hormone therapy, radiation therapy).

Provided herein is a set of blood samples and a set of corresponding nucleic acid samples isolated from the blood samples, where the blood samples are donated from individuals diagnosed with breast cancer. The sample set often includes blood samples or nucleic acid samples from 100 or more, 150 or more, or 200 or more individuals having breast cancer, and sometimes from 250 or more, 300 or more, 400 or more, or 500 or more individuals. The individuals can have parents from any place of origin, and in an embodiment, the set of samples are extracted from individuals of German paternal and German maternal ancestry. The samples in each set may be selected based upon five or more criteria and/or phenotypes set forth above.

Polymorphic Variants Associated with Breast Cancer

A genetic analysis provided herein linked breast cancer with polymorphic variants in and around a nucleotide sequence located on chromosome three that encodes a Rho family guanine-nucleotide exchange factor polypeptide designated KIAA0861. As used herein, the term “polymorphic site” refers to a region in a nucleic acid at which two or more alternative nucleotide sequences are observed in a significant number of nucleic acid samples from a population of individuals. A polymorphic site may be a nucleotide sequence of two or more nucleotides, an inserted nucleotide or nucleotide sequence, a deleted nucleotide or nucleotide sequence, or a microsatellite, for example. A polymorphic site that is two or more nucleotides in length may be 3, 4, 5, 6, 7, 8, 9, 10, 11, 12, 13, 14, 15 or more, 20 or more, 30 or more, 50 or more, 75 or more, 100 or more, 500 or more, or about 1000 nucleotides in length, where all or some of the nucleotide sequences differ within the region. A polymorphic site is often one nucleotide in length, which is referred to herein as a “single nucleotide polymorphism” or a “SNP.”

Where there are two, three, or four alternative nucleotide sequences at a polymorphic site, each nucleotide sequence is referred to as a “polymorphic variant” or “nucleic acid variant.” Where two polymorphic variants exist, for example, the polymorphic variant represented in a minority of samples from a population is sometimes referred to as a “minor allele” and the polymorphic variant that is more prevalently represented is sometimes referred to as a “major allele.” Many organisms possess a copy of each chromosome (e.g., humans), and those individuals who possess two major alleles or two minor alleles are often referred to as being “homozygous” with respect to the polymorphism, and those individuals who possess one major allele and one minor allele are normally referred to as being “heterozygous” with respect to the polymorphism. Individuals who are homozygous with respect to one allele are sometimes predisposed to a different phenotype as compared to individuals who are heterozygous or homozygous with respect to another allele.

Furthermore, a genotype or polymorphic variant may be expressed in terms of a “haplotype,” which as used herein refers to two or more polymorphic variants occurring within genomic DNA in a group of individuals within a population. For example, two SNPs may exist within a gene where each SNP position includes a cytosine variation and an adenine variation. Certain individuals in a population may carry one allele (heterozygous) or two alleles (homozygous) having the gene with a cytosine at each SNP position. As the two cytosines corresponding to each SNP in the gene travel together on one or both alleles in these individuals, the individuals can be characterized as having a cytosine/cytosine haplotype with respect to the two SNPs in the gene.

As used herein, the term “phenotype” refers to a trait which can be compared between individuals, such as presence or absence of a condition, a visually observable difference in appearance between individuals, metabolic variations, physiological variations, variations in the function of biological molecules, and the like. An example of a phenotype is occurrence of breast cancer.

Researchers sometimes report a polymorphic variant in a database without determining whether the variant is represented in a significant fraction of a population. Because a subset of these reported polymorphic variants are not represented in a statistically significant portion of the population, some of them are sequencing errors and/or not biologically relevant. Thus, it is often not known whether a reported polymorphic variant is statistically significant or biologically relevant until the presence of the variant is detected in a population of individuals and the frequency of the variant is determined. Methods for detecting a polymorphic variant in a population are described herein, specifically in Example 2. A polymorphic variant is statistically significant and often biologically relevant if it is represented in 5% or more of a population, sometimes 10% or more, 15% or more, or 20% or more of a population, and often 25% or more, 30% or more, 35% or more, 40% or more, 45% or more, or 50% or more of a population.

A polymorphic variant may be detected on either or both strands of a double-stranded nucleic acid. For example, a thymine at a particular position in SEQ ID NO: 1 can be reported as an adenine from the complementary strand. Also, a polymorphic variant may be located within an intron or exon of a gene or within a portion of a regulatory region such as a promoter, a 5′ untranslated region (UTR), a 3′ UTR, and in DNA (e.g., genomic DNA (gDNA) and complementary DNA (cDNA)), RNA (e.g., mRNA, tRNA, and rRNA), or a polypeptide. Polymorphic variations may or may not result in detectable differences in gene expression, polypeptide structure, or polypeptide function.

In the genetic analysis that associated breast cancer with the polymorphic variants described hereafter, samples from individuals having breast cancer and individuals not having cancer were allelotyped and genotyped. The term “genotyped” as used herein refers to a process for determining a genotype of one or more individuals, where a “genotype” is a representation of one or more polymorphic variants in a population. Genotypes may be expressed in terms of a “haplotype,” which as used herein refers to two or more polymorphic variants occurring within genomic DNA in a group of individuals within a population. For example, two SNPs may exist within a gene where each SNP position includes a cytosine variation and an adenine variation. Certain individuals in a population may carry one allele (heterozygous) or two alleles (homozygous) having the gene with a cytosine at each SNP position. As the two cytosines corresponding to each SNP in the gene travel together on one or both alleles in these individuals, the individuals can be characterized as having a cytosine/cytosine haplotype with respect to the two SNPs in the gene.

It was determined that polymorphic variations associated with an increased risk of breast cancer existed in KIAA0861 nucleotide sequences. Polymorphic variants in and around the KIAA0861 locus were tested for an association with breast cancer. These included polymorphic variants at positions selected from the group consisting of rs3811728, rs3811729, rs602646, rs488277, rs1629673, rs670232, rs575326, rs575386, rs684846, rs471365, rs496251, rs831246, rs831247, rs512071, rs1502761, rs681516, rs683302, rs619424, rs620722, rs529055, rs664010, rs678454, rs2653845, rs472795, rs507079, rs534333, rs535298, rs536213, rs831245, rs639690, rs684174, rs571761, rs1983421, rs4630966, rs2314415, rs6788196, rs2103062, rs9827084, rs9864865, rs6804951, rs6770548, rs1403452, rs7609994, rs9838250, rs9863404, rs903950, rs6787284, rs2017340, rs2001449, rs1317288, rs7635891, rs10704581, rs11371910, rs10937118, rs7642053, rs3821522, rs2029926, rs1390831, rs7643890, rs11925606, rs9826325, rs6800429, rs6803368, rs1353566, rs2272115, rs2272116, rs3732603, rs940055, rs2314730, rs2030578, rs2049280, rs3732602, rs2293203 and rs7639705; and position 13507 of SEQ ID NO: 1. These positions correspond to positions 246, 393, 628, 7586, 9223, 9933, 10154, 10175, 10877, 10907, 11289, 11793, 11813, 14249, 14586, 14647, 15004, 16573, 16811, 18921, 19651, 20565, 25239, 25721, 27133, 27778, 27906, 28000, 30005, 30520, 32195, 32439, 33858, 41716, 42450, 43554, 44211, 44775, 44962, 45317, 45712, 45941, 46520, 47175, 48045, 48636, 48689, 48704, 48849, 48850, 49931, 51510, 51526, 51758, 51975, 53475, 55524, 56754, 57473, 57497, 57613, 58023, 58821, 59644, 66217, 66344, 67326, 69777, 83594, 84579, 85623 and 13507 in SEQ ID NO: 1, respectively. Polymorphic variants in a region spanning positions 14647 to 48849 in SEQ ID NO: 1 were in particular associated with an increased risk of breast cancer, including polymorphic variants at positions 41716, 44775, 44962, 45317, 45712, 45941, and 48849 in SEQ ID NO: 1 (i.e., positions designated by rs4630966, rs9827084, rs9864865, rs6804951, rs6770548, rs1403452 and rs2001449, respectively). At these positions in SEQ ID NO: 1, a cytosine at position 41716, a guanine at position 44775, a guanine at position 44962, a cytosine at position 45317, a guanine at position 45712, a thymine at position 45941, and a cytosine at position 48849 were in particular associated with an increased risk of breast cancer. Also, an alanine at amino acid position 819 in SEQ ID NO: 4 (or position 902 in SEQ ID NO: 5) was in particular associated with an increased risk of breast cancer.

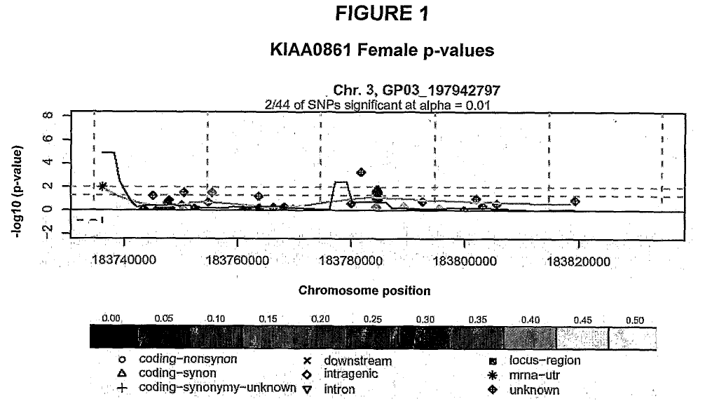

Based in part upon analyses summarized in FIG. 1, a region with significant association has been identified in a KIAA0861 region associated with increased risk of breast cancer. Any polymorphic variants associated with an increased risk of breast cancer in a region of significant association can be utilized for embodiments described herein. The following reports such a region, where “begin” and “end” designate the boundaries of the region according to chromosome positions within NCBI's Genome build 34. The chromosome on which the KIAA0861 region resides and an incident polymorphism in the locus also are noted.

| Incident | chr | begin | End | size |
|---|---|---|---|---|
| 2001449 | 3 | 184215647 | 184249849 | 34202 |

The polymorphic variants described above and in the Examples section are applicable to embodiments described hereafter.

Additional Polymorphic Variants Associated with Breast Cancer

Also provided is a method for identifying polymorphic variants proximal to an incident, founder polymorphic variant associated with breast cancer. Thus, featured herein are methods for identifying a polymorphic variation associated with breast cancer that is proximal to an incident polymorphic variation associated with breast cancer, which comprises identifying a polymorphic variant proximal to the incident polymorphic variant associated with breast cancer, where the incident polymorphic variant is in a nucleotide sequence set forth in SEQ ID NO: 1. The nucleotide sequence often comprises a polynucleotide sequence selected from the group consisting of (a) a nucleotide sequence set forth in SEQ ID NO: 1; (b) a nucleotide sequence which encodes a polypeptide having an amino acid sequence encoded by a nucleotide sequence in SEQ ID NO: 1; (c) a nucleotide sequence which encodes a polypeptide that is 90% or more identical to an amino acid sequence encoded by a nucleotide sequence in SEQ ID NO: 1 or a nucleotide sequence about 90% or more identical to the nucleotide sequence set forth in SEQ ID NO: 1; and (d) a fragment of a nucleotide sequence of (a), (b), or (c), often a fragment that includes a polymorphic site associated with breast cancer. The presence or absence of an association of the proximal polymorphic variant with breast cancer then is determined using a known association method, such as a method described in the Examples hereafter. In an embodiment, the incident polymorphic variant is set forth in SEQ ID NO: 1. In another embodiment, the proximal polymorphic variant identified sometimes is a publicly disclosed polymorphic variant, which for example, sometimes is published in a publicly available database. In other embodiments, the polymorphic variant identified is not publicly disclosed and is discovered using a known method, including, but not limited to, sequencing a region surrounding the incident polymorphic variant in a group of nucleic samples. Thus, multiple polymorphic variants proximal to an incident polymorphic variant are associated with breast cancer using this method.

The proximal polymorphic variant often is identified in a region surrounding the incident polymorphic variant. In certain embodiments, this surrounding region is about 50 kb flanking the first polymorphic variant (e.g. about 50 kb 5′ of the first polymorphic variant and about 50 kb 3′ of the first polymorphic variant), and the region sometimes is composed of shorter flanking sequences, such as flanking sequences of about 40 kb, about 30 kb, about 25 kb, about 20 kb, about 15 kb, about 10 kb, about 7 kb, about 5 kb, or about 2 kb 5′ and 3′ of the incident polymorphic variant. In other embodiments, the region is composed of longer flanking sequences, such as flanking sequences of about 55 kb, about 60 kb, about 65 kb, about 70 kb, about 75 kb, about 80 kb, about 85 kb, about 90 kb, about 95 kb, or about 100 kb 5′ and 3′ of the incident polymorphic variant.

In certain embodiments, polymorphic variants associated with breast cancer are identified iteratively. For example, a first proximal polymorphic variant is associated with breast cancer using the methods described above and then another polymorphic variant proximal to the first proximal polymorphic variant is identified (e.g., publicly disclosed or discovered) and the presence or absence of an association of one or more other polymorphic variants proximal to the first proximal polymorphic variant with breast cancer is determined.

The methods described herein are useful for identifying or discovering additional polymorphic variants that may be used to further characterize a gene, region or loci associated with a condition, a disease (e.g., breast cancer), or a disorder. For example, allelotyping or genotyping data from the additional polymorphic variants may be used to identify a functional mutation or a region of linkage disequilibrium.

In certain embodiments, polymorphic variants identified or discovered within a region comprising the first polymorphic variant associated with breast cancer are genotyped using the genetic methods and sample selection techniques described herein, and it can be determined whether those polymorphic variants are in linkage disequilibrium with the first polymorphic variant. The size of the region in linkage disequilibrium with the first polymorphic variant also can be assessed using these genotyping methods. Thus, provided herein are methods for determining whether a polymorphic variant is in linkage disequilibrium with a first polymorphic variant associated with breast cancer, and such information can be used in prognosis methods described herein.

Isolated KIAA0861 Nucleic Acids

Featured herein are isolated KIAA0861 nucleic acids, which include the nucleic acid having the nucleotide sequence of SEQ ID NO: 1, 2 or 3, nucleic acid variants, and substantially identical nucleic acids of the foregoing. Nucleotide sequences of the KIAA0861 nucleic acids sometimes are referred to herein as “KIAA0861 nucleotide sequences.” A “KIAA0861 nucleic acid variant” refers to one allele that may have one or more different polymorphic variations as compared to another allele in another subject or the same subject. A polymorphic variation in the KIAA0861 nucleic acid variant may be represented on one or both strands in a double-stranded nucleic acid or on one chromosomal complement (heterozygous) or both chromosomal complements (homozygous).

As used herein, the term “nucleic acid” includes DNA molecules (e.g., a complementary DNA (cDNA) and genomic DNA (gDNA)) and RNA molecules (e.g., mRNA, rRNA, and tRNA) and analogs of DNA or RNA, for example, by use of nucleotide analogs. The nucleic acid molecule can be single-stranded and it is often double-stranded. The term “isolated or purified nucleic acid” refers to nucleic acids that are separated from other nucleic acids present in the natural source of the nucleic acid. For example, with regard to genomic DNA, the term “isolated” includes nucleic acids which are separated from the chromosome with which the genomic DNA is naturally associated. An “isolated” nucleic acid is often free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. As used herein, the term “KIAA0861 gene” refers to a nucleotide sequence that encodes a KIAA0861 polypeptide.

In particular embodiments, a nucleic acid comprises a polymorphic variation corresponding to position 13507 of SEQ ID NO: 1. The nucleic acid often comprises a part of or all of a nucleotide sequence in SEQ ID NO: 1, 2 and/or 3, or a substantially identical sequence thereof and sometimes such a nucleotide sequence is a 5′ and/or 3′ sequence flanking a polymorphic variant described above that is 5, 6, 7 . . . 50, 51, 52 . . . 100, 101, 102 . . . 500, 501, 502 . . . 999 or 1000 nucleotides in length. Other embodiments are directed to methods of identifying a polymorphic variation at one or more positions in a nucleic acid (e.g., genotyping at one or more positions in the nucleic acid), where a position corresponds to position 13507 of SEQ ID NO: 1.

Also included herein are nucleic acid fragments. These fragments typically are a nucleotide sequence identical to a nucleotide sequence in SEQ ID NO: 1, 2 or 3, a nucleotide sequence substantially identical to a nucleotide sequence in SEQ ID NO: 1, 2 or 3, or a nucleotide sequence that is complementary to the foregoing. The nucleic acid fragment may be identical, substantially identical or homologous to a nucleotide sequence in an exon or an intron in SEQ ID NO: 1, and may encode a domain or part of a domain or motif of a KIAA0861 polypeptide. Domains and motifs of a KIAA0861 polypeptide include, but are not limited to, a Sec 14p-like lipid binding domain, spectrin repeats (SPEC), a RhoGEF domain (also called the DBL-homology domain (DH domain)), and a Pleckstrin-homology domain (PH domain). Sometimes, the fragment will comprises the polymorphic variation described herein as being associated with breast cancer. The nucleic acid fragment sometimes is 50, 100, or 200 or fewer base pairs in length, and is sometimes about 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3800, 4000, 5000, 6000, 7000, 8000, 9000, 10000, 15000, 20000, 30000, 40000, 50000, 60000, 70000, 80000, 90000, 100000, 110000, 120000, 130000, 140000, 150000 or 160000 base pairs in length. A nucleic acid fragment complementary to a nucleotide sequence identical or substantially identical to the nucleotide sequence of SEQ ID NO: 1, 2 or 3 and hybridizes to such a nucleotide sequence under stringent conditions often is referred to as a “probe.” Nucleic acid fragments often include one or more polymorphic sites, or sometimes have an end that is adjacent to a polymorphic site as described hereafter.

An example of a nucleic acid fragment is an oligonucleotide. As used herein, the term “oligonucleotide” refers to a nucleic acid comprising about 8 to about 50 covalently linked nucleotides, often comprising from about 8 to about 35 nucleotides, and more often from about 10 to about 25 nucleotides. The backbone and nucleotides within an oligonucleotide may be the same as those of naturally occurring nucleic acids, or analogs or derivatives of naturally occurring nucleic acids, provided that oligonucleotides having such analogs or derivatives retain the ability to hybridize specifically to a nucleic acid comprising a targeted polymorphism. Oligonucleotides described herein may be used as hybridization probes or as components of prognostic or diagnostic assays, for example, as described herein.

Oligonucleotides are typically synthesized using standard methods and equipment, such as the ABI 3900 High Throughput DNA Synthesizer and the EXPEDITE™ 8909 Nucleic Acid Synthesizer, both of which are available from Applied Biosystems (Foster City, Calif.). Analogs and derivatives are exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WO 00/56746; WO 01/14398, and related publications. Methods for synthesizing oligonucleotides comprising such analogs or derivatives are disclosed, for example, in the patent publications cited above and in U.S. Pat. Nos. 5,614,622; 5,739,314; 5,955,599; 5,962,674; 6,117,992; in WO 00/75372; and in related publications.

Oligonucleotides also may be linked to a second moiety. The second moiety may be an additional nucleotide sequence such as a tail sequence (e.g., a polyadenosine tail), an adapter sequence (e.g., phage M13 universal tail sequence), and others. Alternatively, the second moiety may be a non-nucleotide moiety such as a moiety which facilitates linkage to a solid support or a label to facilitate detection of the oligonucleotide. Such labels include, without limitation, a radioactive label, a fluorescent label, a chemiluminescent label, a paramagnetic label, and the like. The second moiety may be attached to any position of the oligonucleotide, provided the oligonucleotide can hybridize to the nucleic acid comprising the polymorphism.

### Uses for Nucleic Acid Sequences

Nucleic acid coding sequences depicted in SEQ ID NO: 2 or 3 may be used for diagnostic purposes for detection and control of polypeptide expression. Also, included herein are oligonucleotide sequences such as antisense RNA, small-interfering RNA (siRNA) and DNA molecules and ribozymes that function to inhibit translation of a polypeptide. Antisense techniques and RNA interference techniques are known in the art and are described herein.

Ribozymes are enzymatic RNA molecules capable of catalyzing the specific cleavage of RNA. The mechanism of ribozyme action involves sequence specific hybridization of the ribozyme molecule to complementary target RNA, followed by a endonucleolytic cleavage. Ribozymes may be engineered hammerhead motif ribozyme molecules that specifically and efficiently catalyze endonucleolytic cleavage of RNA sequences corresponding to or complementary to the nucleotide sequences set forth in SEQ ID NO: 1-3. Specific ribozyme cleavage sites within any potential RNA target are initially identified by scanning the target molecule for ribozyme cleavage sites which include the following sequences, GUA, GUU and GUC. Once identified, short RNA sequences of between fifteen (15) and twenty (20) ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Antisense RNA and DNA molecules, siRNA and ribozymes may be prepared by any method known in the art for the synthesis of RNA molecules. These include techniques for chemically synthesizing oligodeoxyribonucleotides well known in the art such as solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

DNA encoding a polypeptide also may have a number of uses for the diagnosis of diseases, including breast cancer, resulting from aberrant expression of a target gene described herein. For example, the nucleic acid sequence may be used in hybridization assays of biopsies or autopsies to diagnose abnormalities of expression or function (e.g., Southern or Northern blot analysis, in situ hybridization assays).

In addition, the expression of a polypeptide during embryonic development may also be determined using nucleic acid encoding the polypeptide. As addressed, infra, production of functionally impaired polypeptide can be the cause of various disease states, such as breast cancer. In situ hybridizations using polynucleotide probes may be employed to predict problems related to breast cancer. Further, as indicated, infra, administration of human active polypeptide, recombinantly produced as described herein, may be used to treat disease states related to functionally impaired polypeptide (e.g., a KIAA0861 polypeptide that activates a Rho GTPase in a situation where it is not normally activated). Alternatively, gene therapy approaches may be employed to remedy deficiencies of functional polypeptide or to replace or compete with dysfunctional polypeptide.

Expression Vectors, Host Cells, and Genetically Engineered Cells

Provided herein are nucleic acid vectors, often expression vectors, which contain a KIAA0861 nucleic acid. As used herein, the term “vector” refers to a nucleic acid molecule capable of transporting another nucleic acid to which it has been linked and can include a plasmid, cosmid, or viral vector. The vector can be capable of autonomous replication or it can integrate into a host DNA. Viral vectors may include replication defective retroviruses, adenoviruses and adeno-associated viruses for example.

A vector can include a KIAA0861 nucleic acid in a form suitable for expression of the nucleic acid in a host cell. The recombinant expression vector typically includes one or more regulatory sequences operatively linked to the nucleic acid sequence to be expressed. The term “regulatory sequence” includes promoters, enhancers and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of polypeptide desired, and the like. Expression vectors can be introduced into host cells to produce KIAA0861 polypeptides, including fusion polypeptides, encoded by KIAA0861 nucleic acids.

Recombinant expression vectors can be designed for expression of KIAA0861 polypeptides in prokaryotic or eukaryotic cells. For example, KIAA0861 polypeptides can be expressed in E. coli, insect cells (e.g., using baculovirus expression vectors), yeast cells, or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: 1) to increase expression of recombinant polypeptide; 2) to increase the solubility of the recombinant polypeptide; and 3) to aid in the purification of the recombinant polypeptide by acting as a ligand in affinity purification. Often, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith & Johnson, Gene 67: 31-40 (1988)), pMAL (New England Biolabs, Beverly, Mass.) and pRIT5 (Pharmacia, Piscataway, N.J.) which fuse glutathione S-transferase (GST), maltose E binding polypeptide, or polypeptide A, respectively, to the target recombinant polypeptide.

Purified fusion polypeptides can be used in screening assays and to generate antibodies specific for KIAA0861 polypeptides. In a therapeutic embodiment, fusion polypeptide expressed in a retroviral expression vector is used to infect bone marrow cells that are subsequently transplanted into irradiated recipients. The pathology of the subject recipient is then examined after sufficient time has passed (e.g., six (6) weeks).

Expressing the polypeptide in host bacteria with an impaired capacity to proteolytically cleave the recombinant polypeptide is often used to maximize recombinant polypeptide expression (Gottesman, S., Gene Expression Technology: Methods in Enzymology, Academic Press, San Diego, Calif. 185: 119-128 (1990)). Another strategy is to alter the nucleotide sequence of the nucleic acid to be inserted into an expression vector so that the individual codons for each amino acid are those preferentially utilized in E. coli (Wada et al., Nucleic Acids Res. 20: 2111-2118 (1992)). Such alteration of nucleotide sequences can be carried out by standard DNA synthesis techniques.

When used in mammalian cells, the expression vector's control functions are often provided by viral regulatory elements. For example, commonly used promoters are derived from polyoma, Adenovirus 2, cytomegalovirus and Simian Virus 40. Recombinant mammalian expression vectors are often capable of directing expression of the nucleic acid in a particular cell type (e.g., tissue-specific regulatory elements are used to express the nucleic acid). Non-limiting examples of suitable tissue-specific promoters include an albumin promoter (liver-specific; Pinkert et al., Genes Dev. 1: 268-277 (1987)), lymphoid-specific promoters (Calame & Eaton, Adv. Immunol. 43: 235-275 (1988)), promoters of T cell receptors (Winoto & Baltimore, EMBO J. 8: 729-733 (1989)) promoters of immunoglobulins (Banerji et al., Cell 33: 729-740 (1983); Queen & Baltimore, Cell 33: 741-748 (1983)), neuron-specific promoters (e.g., the neurofilament promoter; Byrne & Ruddle, Proc. Natl. Acad. Sci. USA 86: 5473-5477 (1989)), pancreas-specific promoters (Edlund et al., Science 230: 912-916 (1985)), and mammary gland-specific promoters (e.g., milk whey promoter; U.S. Pat. No. 4,873,316 and European Application Publication No. 264,166). Developmentally-regulated promoters are sometimes utilized, for example, the murine hox promoters (Kessel & Gruss, Science 249: 374-379 (1990)) and the α-fetopolypeptide promoter (Campes & Tilghman, Genes Dev. 3: 537-546 (1989)).

A KIAA0861 nucleic acid may also be cloned into an expression vector in an antisense orientation. Regulatory sequences (e.g., viral promoters and/or enhancers) operatively linked to a KIAA0861 nucleic acid cloned in the antisense orientation can be chosen for directing constitutive, tissue specific or cell type specific expression of antisense RNA in a variety of cell types. Antisense expression vectors can be in the form of a recombinant plasmid, phagemid or attenuated virus. For a discussion of the regulation of gene expression using antisense genes see Weintraub et al., Antisense RNA as a molecular tool for genetic analysis, Reviews—Trends in Genetics, Vol. 1(1) (1986).

Also provided herein are host cells that include a KIAA0861 nucleic acid within a recombinant expression vector or KIAA0861 nucleic acid sequence fragments which allow it to homologously recombine into a specific site of the host cell genome. The terms “host cell” and “recombinant host cell” are used interchangeably herein. Such terms refer not only to the particular subject cell but rather also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a KIAA0861 polypeptide can be expressed in bacterial cells such as E. coli, insect cells, yeast or mammalian cells (such as Chinese hamster ovary cells (CHO) or COS cells). Other suitable host cells are known to those skilled in the art.

Vectors can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, transduction/infection, DEAE-dextran-mediated transfection, lipofection, or electroporation.

A host cell provided herein can be used to produce (i.e., express) a KIAA0861 polypeptide. Accordingly, further provided are methods for producing a KIAA0861 polypeptide using the host cells described herein. In one embodiment, the method includes culturing host cells into which a recombinant expression vector encoding a KIAA0861 polypeptide has been introduced in a suitable medium such that a KIAA0861 polypeptide is produced. In another embodiment, the method further includes isolating a KIAA0861 polypeptide from the medium or the host cell.

Also provided are cells or purified preparations of cells which include a KIAA0861 transgene, or which otherwise misexpress KIAA0861 polypeptide. Cell preparations can consist of human or non-human cells, e.g., rodent cells, e.g., mouse or rat cells, rabbit cells, or pig cells. In certain embodiments, the cell or cells include a KIAA0861 transgene (e.g., a heterologous form of a KIAA0861 such as a human gene expressed in non-human cells). The KIAA0861 transgene can be misexpressed, e.g., overexpressed or underexpressed. In other embodiments, the cell or cells include a gene which misexpress an endogenous KIAA0861 polypeptide (e.g., expression of a gene is disrupted, also known as a knockout). Such cells can serve as a model for studying disorders which are related to mutated or mis-expressed KIAA0861 alleles or for use in drug screening. Also provided are human cells (e.g., a hematopoietic stem cells) transformed with a KIAA0861 nucleic acid.

Also provided are cells or a purified preparation thereof (e.g., human cells) in which an endogenous KIAA0861 nucleic acid is under the control of a regulatory sequence that does not normally control the expression of the endogenous KIAA0861 gene. The expression characteristics of an endogenous gene within a cell (e.g., a cell line or microorganism) can be modified by inserting a heterologous DNA regulatory element into the genome of the cell such that the inserted regulatory element is operably linked to the endogenous KIAA0861 gene. For example, an endogenous KIAA0861 gene (e.g., a gene which is “transcriptionally silent,” not normally expressed, or expressed only at very low levels) may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published on May 16, 1991.

Transgenic Animals

Non-human transgenic animals that express a heterologous KIAA0861 polypeptide (e.g., expressed from a KIAA0861 nucleic acid isolated from another organism) can be generated. Such animals are useful for studying the function and/or activity of a KIAA0861 polypeptide and for identifying and/or evaluating modulators of KIAA0861 nucleic acid and KIAA0861 polypeptide activity. As used herein, a “transgenic animal” is a non-human animal such as a mammal (e.g., a non-human primate such as chimpanzee, baboon, or macaque; an ungulate such as an equine, bovine, or caprine; or a rodent such as a rat, a mouse, or an Israeli sand rat), a bird (e.g., a chicken or a turkey), an amphibian (e.g., a frog, salamander, or newt), or an insect (e.g., Drosophila melanogaster), in which one or more of the cells of the animal includes a KIAA0861 transgene. A transgene is exogenous DNA or a rearrangement (e.g., a deletion of endogenous chromosomal DNA) that is often integrated into or occurs in the genome of cells in a transgenic animal. A transgene can direct expression of an encoded gene product in one or more cell types or tissues of the transgenic animal, and other transgenes can reduce expression (e.g., a knockout). Thus, a transgenic animal can be one in which an endogenous KIAA0861 gene has been altered by homologous recombination between the endogenous gene and an exogenous DNA molecule introduced into a cell of the animal (e.g., an embryonic cell of the animal) prior to development of the animal.

Intronic sequences and polyadenylation signals can also be included in the transgene to increase expression efficiency of the transgene. One or more tissue-specific regulatory sequences can be operably linked to a KIAA0861 transgene to direct expression of a KIAA0861 polypeptide to particular cells. A transgenic founder animal can be identified based upon the presence of a KIAA0861 transgene in its genome and/or expression of KIAA0861 mRNA in tissues or cells of the animals. A transgenic founder animal can then be used to breed additional animals carrying the transgene. Moreover, transgenic animals carrying a transgene encoding a KIAA0861 polypeptide can further be bred to other transgenic animals carrying other transgenes.

KIAA0861 polypeptides can be expressed in transgenic animals or plants by introducing, for example, a nucleic acid encoding the polypeptide into the genome of an animal. In certain embodiments the nucleic acid is placed under the control of a tissue specific promoter, e.g., a milk or egg specific promoter, and recovered from the milk or eggs produced by the animal. Also included is a population of cells from a transgenic animal.

KIAA0861 Polypeptides

Featured herein are isolated KIAA0861 polypeptides, which include polypeptides having amino acid sequences of SEQ ID NO: 4 or 5, and substantially identical polypeptides thereof. Such polypeptides sometimes are proteins or peptides. The polypeptide having the amino acid sequence of SEQ ID NO: 5 often is utilized, as well as domain fragments, such as a fragment containing the DH and PH domains. A KIAA0861 polypeptide is a polypeptide encoded by a KIAA0861 nucleic acid, where one nucleic acid can encode one or more different polypeptides. An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of a KIAA0861 polypeptide or KIAA0861 polypeptide variant having less than about 30%, 20%, 10% and sometimes 5% (by dry weight), of non-KIAA0861 polypeptide (also referred to herein as a “contaminating protein”), or of chemical precursors or non-KIAA0861 chemicals. When the KIAA0861 polypeptide or a biologically active portion thereof is recombinantly produced, it is also often substantially free of culture medium, specifically, where culture medium represents less than about 20%, sometimes less than about 10%, and often less than about 5% of the volume of the polypeptide preparation. Isolated or purified KIAA0861 polypeptide preparations are sometimes 0.01 milligrams or more or 0.1 milligrams or more, and often 1.0 milligrams or more and 10 milligrams or more in dry weight. In specific embodiments, the KIAA0861 polypeptide comprises a leucine at amino acid position 359 in SEQ ID NO: 5, a leucine at amino acid position 378 in SEQ ID NO: 5, or an alanine at amino acid position 857 in SEQ ID NO: 5.

In another aspect, featured herein are KIAA0861 polypeptides and biologically active or antigenic fragments thereof that are useful as reagents or targets in assays applicable to prevention, treatment or diagnosis of breast cancer. In another embodiment, provided herein are KIAA0861 polypeptides having a KIAA0861 activity or activities (e.g., GTPase binding activity, guanine nucleotide exchange activity (i.e., the ability to catalyze GDP-GTP exchange reactions of Rho proteins), translocating the GEF to the plasma membrane activity (i.e., cellular localization via interactions with lipids or proteins), or recognizing the substrate GTPase activity). In certain embodiments, the polypeptides are KIAA0861 proteins including a Sec 14p-like lipid binding domain, at least one spectrin repeat (SPEC), a RhoGEF domain (or DH domain), and a Pleckstrin-homology domain (PH domain), and sometimes having a KIAA0861 activity as described herein. These domains are always found in tandem, with the PH domain found C-terminal to the DH domain. It is believed that the DH domain interacts directly with Rho GTPase depleted of GTP and Mg²⁺ while the PH domain is responsible for translocating the GEF to the plasma membrane, placing it in close proximity to the substrate GTPase. A second, or alternative role for the PH domain has recently been described and involves the direct interaction of the PH domain with the GTPase (Rossman, K L, et al. (2002) EMBO, 21 (6): 1315-1326). Methods for monitoring and quantifying this biological activity, both in vitro and in vivo, are known (see, e.g., Cheng, L, et al. (2002) MCB. 22 (19):6895-6905).

A catalytically active form of the KIAA0861 protein includes the RhoGEF domain (DH domain), which serves to catalyze GDP-GTP exchange reactions of Rho proteins, and the Pleckstrin-homology domain (PH domain), which serves to translocate the GEF to the plasma membrane. The catalytically active form of the KIAA0861 protein can be approximately 294, 295, 296, 297, 298, 299, 300, 301, 302, 303, 304, 305, 330, 331, 332, 333, 334, 335, 336, 337, 338, 339, 340, or 341 amino acid residues in length (from about amino acid 535, 536, 537, 538, 539, 540, 571, 572, 573, 574, 575 or 576 to amino acid 870, 871, 872, 873, 874, 875 or 876 of SEQ ID NO: 5).

Human KIAA0861 contains the following regions or other structural features: a Sec14p-like lipid binding domain at about amino acids 99 to 190 of SEQ ID NO: 5; Spectrin repeats located at about amino acid residues 333 to 503 of SEQ ID NO: 5; RhoGEF (or DH) domain at about amino acids 623 to 820 or 659 to 818 of SEQ ID NO: 5; and a Pleckstrin-homology domain (PH domain) at about amino acids 857-953 or 857-956 of SEQ ID NO: 5. DH-PH domains often span from amino acids 623-953 or 623-856 in SEQ ID NO: 5. A nucleotide sequence of a DBS gene, another guanine nucleotide exchange factor discussed hereafter, is deposited as NP_{—}079255 in the GenBank database and DB-PH regions corresponding to the KIAA0861 DB-PH region are apparent from alignments shown hereafter.

In other embodiments, there are provided methods of decreasing the guanine nucleotide exchange reactions of Rho proteins, comprising providing or administering to individuals in need of decreasing the guanine nucleotide exchange reactions of Rho proteins the pharmaceutical or physiologically acceptable composition comprising inactive human KIAA0861 protein or fragment thereof, where the inactive KIAA0861 polypeptide fragments may have introduced point mutations in the DH domain of KIAA0861 to selectively narrow its specificity of exchange, further wherein it is understood that the inactive form of KIAA0861 does not have the ability or has a decreased ability to catalyze the guanine nucleotide exchange reactions of Rho proteins, but can still bind to Rho proteins. (See “Therapeutic Treatments” Section herein).

Further included herein are KIAA0861 polypeptide fragments. The polypeptide fragment may be a domain or part of a domain of a KIAA0861 polypeptide. The polypeptide fragment is often 50 or fewer, 100 or fewer, or 200 or fewer amino acids in length, and is sometimes 300, 400, 500, 600, 700, or 900 or fewer amino acids in length. In certain embodiments, the polypeptide fragment comprises, consists essentially of, or consists of, at least 6 consecutive amino acids and not more than 1211 consecutive amino acids of SEQ ID NO: 5, or the polypeptide fragment comprises, consists essentially of, or consists of, at least 6 consecutive amino acids and not more than 543 consecutive amino acids of SEQ ID NO: 5.

KIAA0861 polypeptides described herein can be used as immunogens to produce anti-KIAA0861 antibodies in a subject, to purify KIAA0861 ligands or binding partners, and in screening assays to identify molecules which inhibit or enhance the interaction of KIAA0861 with a KIAA0861 substrate. In a preferred embodiment, KIAA0861 polypeptides described herein are used to screen for competitive inhibitors of KIAA0861 with Rho family GTP-binding proteins. Full-length KIAA0861 polypeptides and polynucleotides encoding the same may be specifically substituted for a KIAA0861 polypeptide fragment or polynucleotide encoding the same in any embodiment described herein.

Substantially identical polypeptides may depart from the amino acid sequences of SEQ ID NO: 4 or 5 in different manners. For example, conservative amino acid modifications may be introduced at one or more positions in the amino acid sequences of SEQ ID NO: 4 or 5. A “conservative amino acid substitution” is one in which the amino acid is replaced by another amino acid having a similar structure and/or chemical function. Families of amino acid residues having similar structures and functions are well known. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Also, essential and non-essential amino acids may be replaced. A “non-essential” amino acid is one that can be altered without abolishing or substantially altering the biological function of a KIAA0861 polypeptide, whereas altering an “essential” amino acid abolishes or substantially alters the biological function of a KIAA0861 polypeptide. Amino acids that are conserved among KIAA0861 polypeptides are typically essential amino acids.

Also, KIAA0861 polypeptides and polypeptide variants may exist as chimeric or fusion polypeptides. As used herein, a KIAA0861 “chimeric polypeptide” or “fusion polypeptide” includes a KIAA0861 polypeptide linked to a non-KIAA0861 polypeptide. A “non-KIAA0861 polypeptide” refers to a polypeptide having an amino acid sequence corresponding to a polypeptide which is not substantially identical to the KIAA0861 polypeptide, which includes, for example, a polypeptide that is different from the KIAA0861 polypeptide and derived from the same or a different organism. The KIAA0861 polypeptide in the fusion polypeptide can correspond to an entire or nearly entire KIAA0861 polypeptide or a fragment thereof. The non-KIAA0861 polypeptide can be fused to the N-terminus or C-terminus of the KIAA0861 polypeptide.

Fusion polypeptides can include a moiety having high affinity for a ligand. For example, the fusion polypeptide can be a GST-KIAA0861 fusion polypeptide in which the KIAA0861 sequences are fused to the C-terminus of the GST sequences, or a polyhistidine-KIAA0861 fusion polypeptide in which the KIAA0861 polypeptide is fused at the N- or C-terminus to a string of histidine residues. Such fusion polypeptides can facilitate purification of recombinant KIAA0861. Expression vectors are commercially available that already encode a fusion moiety (e.g., a GST polypeptide), and a KIAA0861 nucleic acid can be cloned into an expression vector such that the fusion moiety is linked in-frame to the KIAA0861 polypeptide. Further, the fusion polypeptide can be a KIAA0861 polypeptide containing a heterologous signal sequence at its N-terminus. In certain host cells (e.g., mammalian host cells), expression, secretion, cellular internalization, and cellular localization of a KIAA0861 polypeptide can be increased through use of a heterologous signal sequence. Fusion polypeptides can also include all or a part of a serum polypeptide (e.g., an IgG constant region or human serum albumin).

KIAA0861 polypeptides or fragments thereof can be incorporated into pharmaceutical compositions and administered to a subject in vivo. Administration of these KIAA0861 polypeptides can be used to affect the bioavailability of a KIAA0861 substrate and may effectively increase or decrease KIAA0861 biological activity in a cell or effectively supplement dysfunctional or hyperactive KIAA0861 polypeptide. KIAA0861 fusion polypeptides may be useful therapeutically for the treatment of disorders caused by, for example, (i) aberrant modification or mutation of a gene encoding a KIAA0861 polypeptide; (ii) mis-regulation of the KIAA0861 gene; and (iii) aberrant post-translational modification of a KIAA0861 polypeptide. Also, KIAA0861 polypeptides can be used as immunogens to produce anti-KIAA0861 antibodies in a subject, to purify KIAA0861 ligands or binding partners, and in screening assays to identify molecules which inhibit or enhance the interaction of KIAA0861 with a KIAA0861 substrate. Preferably, said KIAA0861 polypeptides are used in screening assays to identify molecules which inhibit the interaction of KIAA0861 with Rho family GTP-binding proteins.

In addition, polypeptides can be chemically synthesized using techniques known in the art (See, e.g., Creighton, 1983 Proteins. New York, N.Y.: W. H. Freeman and Company; and Hunkapiller et al., (1984) Nature July 12-18; 310(5973):105-11). For example, a relative short polypeptide fragment can be synthesized by use of a peptide synthesizer. Furthermore, if desired, non-classical amino acids or chemical amino acid analogs can be introduced as a substitution or addition into the fragment sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoroamino acids, designer amino acids such as b-methyl amino acids, Ca-methyl amino acids, Na-methyl amino acids, and amino acid analogs in general. Furthermore, the amino acid can be D (dextrorotary) or L (levorotary).

Also included are polypeptide fragments which are differentially modified during or after translation, e.g., by glycosylation, acetylation, phosphorylation, amidation, derivatization by known protecting/blocking groups, proteolytic cleavage, linkage to an antibody molecule or other cellular ligand, and the like. Any of numerous chemical modifications may be carried out by known techniques, including but not limited, to specific chemical cleavage by cyanogen bromide, trypsin, chymotrypsin, papain, V8 protease, NaBH₄; acetylation, formylation, oxidation, reduction; metabolic synthesis in the presence of tunicamycin; and the like.

Additional post-translational modifications include, for example, N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The polypeptide fragments may also be modified with a detectable label, such as an enzymatic, fluorescent, isotopic or affinity label to allow for detection and isolation of the polypeptide.

Also provided are chemically modified polypeptide derivatives that may provide additional advantages such as increased solubility, stability and circulating time of the polypeptide, or decreased immunogenicity. See U.S. Pat. No. 4,179,337. The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polyethylene glycol molecules (or other chemical moieties) should be attached to the polypeptide with consideration of effects on functional or antigenic domains of the polypeptide. There are a number of attachment methods available to those skilled in the art, e.g., EP 0 401 384, herein incorporated by reference (coupling PEG to G-CSF), see also Malik et al (1992) Exp Hematol. September; 20(8): 1028-35, reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues, glutamic acid residues and the C-terminal amino acid residue. Sulfhydryl groups may also be used as a reactive group for attaching the polyethylene glycol molecules. A polymer sometimes is attached at an amino group, such as attachment at the N-terminus or lysine group.

One may specifically desire proteins chemically modified at the N-terminus. Using polyethylene glycol as an illustration of the present composition, one may select from a variety of polyethylene glycol molecules (by molecular weight, branching, and the like), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus may be accomplished by reductive alkylation, which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

Substantially Identical Nucleic Acids and Polypeptides

Nucleotide sequences and polypeptide sequences that are substantially identical to a KIAA0861 nucleotide sequence and the KIAA0861 polypeptide sequences encoded by those nucleotide sequences are included herein. The term “substantially identical” as used herein refers to two or more nucleic acids or polypeptides sharing one or more identical nucleotide sequences or polypeptide sequences, respectively. Included are nucleotide sequences or polypeptide sequences that are 55% or more, 60% or more, 65% or more, 70% or more, 75% or more, 80% or more, 85% or more, 90% or more, 95% or more (each often within a 1%, 2%, 3% or 4% variability) or more identical to the nucleotide sequences in SEQ ID NO: 1, 2 or 3 or the encoded KIAA0861 polypeptide amino acid sequences. One test for determining whether two nucleic acids are substantially identical is to determine the percent of identical nucleotide sequences or polypeptide sequences shared between the nucleic acids or polypeptides.

Calculations of sequence identity are often performed as follows. Sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is sometimes 30% or more, 40% or more, 50% or more, often 60% or more, and more often 70% or more, 80% or more, 90% or more, 90% or more, or 100% of the length of the reference sequence. The nucleotides or amino acids at corresponding nucleotide or polypeptide positions, respectively, are then compared among the two sequences. When a position in the first sequence is occupied by the same nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences.

Comparison of sequences and determination of percent identity between two sequences can be accomplished using a mathematical algorithm. Percent identity between two amino acid or nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444-453 (1970) algorithm which has been incorporated into the GAP program in the GCG software package (available at the http address www.gcg.com), using either a Blossum 62 matrix or a PAM250 matrix, and a gap weight of 16, 14, 12, 10, 8, 6, or 4 and a length weight of 1, 2, 3, 4, 5, or 6. Percent identity between two nucleotide sequences can be determined using the GAP program in the GCG software package (available at http address www.gcg.com), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Another manner for determining if two nucleic acids are substantially identical is to assess whether a polynucleotide homologous to one nucleic acid will hybridize to the other nucleic acid under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C.

An example of a substantially identical nucleotide sequence to a KIAA0861 nucleotide sequence is one that has a different nucleotide sequence but still encodes the same polypeptide sequence encoded by the KIAA0861 nucleotide sequence. Another example is a nucleotide sequence that encodes a polypeptide having a polypeptide sequence that is more than 70% or more identical to, sometimes 75% or more, 80% or more, or 85% or more identical to, and often 90% or more and 95% or more identical to a polypeptide sequence encoded by a KIAA0861 nucleotide sequence.

KIAA0861 nucleotide sequences and KIAA0861 amino acid sequences can be used as “query sequences” to perform a search against public databases to identify other family members or related sequences, for example. Such searches can be performed using the NBLAST and XBLAST programs (version 2.0) of Altschul et al., J. Mol. Biol. 215: 403-10 (1990). BLAST nucleotide searches can be performed with the NBLAST program, score=100, wordlength=12 to obtain nucleotide sequences homologous to nucleotide sequences from SEQ ID NO: 1. BLAST polypeptide searches can be performed with the XBLAST program, score=50, wordlength=3 to obtain amino acid sequences homologous to polypeptides encoded by a KIAA0861 nucleotide sequence. To obtain gapped alignments for comparison purposes, Gapped BLAST can be utilized as described in Altschul et al., Nucleic Acids Res. 25(17): 3389-3402 (1997). When utilizing BLAST and Gapped BLAST programs, default parameters of the respective programs (e.g., XBLAST and NBLAST) can be used (see the http address www.ncbi.nlm.nih.gov).

A nucleic acid that is substantially identical to a KIAA0861 nucleotide sequence may include polymorphic sites at positions equivalent to those described herein when the sequences are aligned. For example, using the alignment procedures described herein, SNPs in a sequence substantially identical to a sequence in SEQ ID NO: 1, 2, or 3 can be identified at nucleotide positions that match (i.e., align) with nucleotides at SNP positions in the nucleotide sequence of SEQ ID NO: 1, 2 or 3. Also, where a polymorphic variation results in an insertion or deletion, insertion or deletion of a nucleotide sequence from a reference sequence can change the relative positions of other polymorphic sites in the nucleotide sequence.

Substantially identical nucleotide and polypeptide sequences include those that are naturally occurring, such as allelic variants (same locus), splice variants, homologs (different locus), and orthologs (different organism) or can be non-naturally occurring. Non-naturally occurring variants can be generated by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). Orthologs, homologs, allelic variants, and splice variants can be identified using methods known in the art. These variants normally comprise a nucleotide sequence encoding a polypeptide that is 50% or more, about 55% or more, often about 70-75% or more, more often about 80-85% or more, and typically about 90-95% or more identical to the amino acid sequences of target polypeptides or a fragment thereof. Such nucleic acid molecules readily can be identified as being able to hybridize under stringent conditions to a nucleotide sequence in SEQ ID NO: 1, 2 or 3 or a fragment thereof. Nucleic acid molecules corresponding to orthologs, homologs, and allelic variants of a nucleotide sequence in SEQ ID NO: 1, 2 or 3 can be identified by mapping the sequence to the same chromosome or locus as the nucleotide sequence in SEQ ID NO: 1, 2 or 3.

Also, substantially identical nucleotide sequences may include codons that are altered with respect to the naturally occurring sequence for enhancing expression of a target polypeptide in a particular expression system. For example, the nucleic acid can be one in which one or more codons are altered, and often 10% or more or 20% or more of the codons are altered for optimized expression in bacteria (e.g., E. coli.), yeast (e.g., S. cervesiae), human (e.g., 293 cells), insect, or rodent (e.g., hamster) cells.

Methods for Identifying Subjects at Risk of Breast Cancer and Breast Cancer Risk in a Subject

Methods for prognosing and diagnosing breast cancer in subjects are provided herein. These methods include detecting the presence or absence of one or more polymorphic variations associated with breast cancer in a nucleotide sequence set forth in SEQ ID NO: 1, or substantially identical sequence thereof, in a sample from a subject, where the presence of a polymorphic variant described herein is indicative of a risk of breast cancer.

Thus, featured herein is a method for detecting a subject at risk of breast cancer or the risk of breast cancer in a subject, which comprises detecting the presence or absence of a polymorphic variation associated with breast cancer at a polymorphic site in a nucleotide sequence set forth in SEQ ID NO: 1 in a nucleic acid sample from a subject, where the nucleotide sequence comprises a polynucleotide sequence selected from the group consisting of: (a) a nucleotide sequence set forth in SEQ ID NO: 1; (b) a nucleotide sequence which encodes a polypeptide having an amino acid sequence encoded by a nucleotide sequence in SEQ ID NO: 1; (c) a nucleotide sequence which encodes a polypeptide that is 90% or more identical to an amino acid sequence encoded by a nucleotide sequence in SEQ ID NO: 1 or a nucleotide sequence about 90% or more identical to the nucleotide sequence set forth in SEQ ID NO: 1; and (d) a fragment of a nucleotide sequence of (a), (b), or (c), often a fragment that includes a polymorphic site associated with breast cancer; whereby the presence of the polymorphic variation is indicative of a risk of breast cancer in the subject. In specific embodiments, the polymorphic variant is detected at a position corresponding to a position selected from the group consisting of rs3811728, rs3811729, rs602646, rs488277, rs1629673, rs670232, rs575326, rs575386, rs684846, rs471365, rs496251, rs831246, rs831247, rs512071, rs1502761, rs681516, rs683302, rs619424, rs620722, rs529055, rs664010, rs678454, rs2653845, rs472795, rs507079, rs534333, rs535298, rs536213, rs831245, rs639690, rs684174, rs571761, rs1983421, rs4630966, rs2314415, rs6788196, rs2103062, rs9827084, rs9864865, rs6804951, rs6770548, rs1403452, rs7609994, rs9838250, rs9863404, rs903950, rs6787284, rs2017340, rs2001449, rs1317288, rs7635891, rs10704581, rs11371910, rs10937118, rs7642053, rs3821522, rs2029926, rs1390831, rs7643890, rs11925606, rs9826325, rs6800429, rs6803368, rs1353566, rs2272115, rs2272116, rs3732603, rs940055, rs2314730, rs2030578, rs2049280, rs3732602, rs2293203, rs7639705, and position 13507 of SEQ ID NO: 1. In certain embodiments, determining the presence of a combination of two or more polymorphic variants associated with breast cancer in one or more nucleotide sequences of the sample (e.g., at ICAM, MAPK10, NUMA1, DPF3, LOC145197 and/or GALE sequences) is determined to identify a subject at risk of breast cancer and/or risk of breast cancer.

A risk of developing aggressive forms of breast cancer likely to metastasize or invade surrounding tissues (e.g., Stage IIIA, IIIB, and IV breast cancers), and subjects at risk of developing aggressive forms of breast cancer also may be identified by the methods described herein. These methods include collecting phenotype information from subjects having breast cancer, which includes the stage of progression of the breast cancer, and performing a secondary phenotype analysis to detect the presence or absence of one or more polymorphic variations associated with a particular stage form of breast cancer. Thus, detecting the presence or absence of one or more polymorphic variations in a KIAA0861 nucleotide sequence associated with a late stage form of breast cancer often is diagnostic of an aggressive form of the cancer.

Results from prognostic tests may be combined with other test results to diagnose breast cancer. For example, prognostic results may be gathered, a patient sample may be ordered based on a determined predisposition to breast cancer, the patient sample is analyzed, and the results of the analysis may be utilized to diagnose breast cancer. Also breast cancer diagnostic methods can be developed from studies used to generate prognostic/diagnostic methods in which populations are stratified into subpopulations having different progressions of breast cancer. In another embodiment, prognostic results may be gathered; a patient's risk factors for developing breast cancer analyzed (e.g., age, race, family history, age of first menstrual cycle, age at birth of first child); and a patient sample may be ordered based on a determined predisposition to breast cancer. In an alternative embodiment, the results from predisposition analyses described herein may be combined with other test results indicative of breast cancer, which were previously, concurrently, or subsequently gathered with respect to the predisposition testing. In these embodiments, the combination of the prognostic test results with other test results can be probative of breast cancer, and the combination can be utilized as a breast cancer diagnostic. The results of any test indicative of breast cancer known in the art may be combined with the methods described herein. Examples of such tests are mammography (e.g., a more frequent and/or earlier mammography regimen may be prescribed); breast biopsy and optionally a biopsy from another tissue; breast ultrasound and optionally an ultrasound analysis of another tissue; breast magnetic resonance imaging (MRI) and optionally an MRI analysis of another tissue; electrical impedance (T-scan) analysis of breast and optionally of another tissue; ductal lavage; nuclear medicine analysis (e.g., scintimammography); BRCA1 and/or BRCA2 sequence analysis results; and thermal imaging of the breast and optionally of another tissue. Testing may be performed on tissue other than breast to diagnose the occurrence of metastasis (e.g., testing of the lymph node).

Risk of breast cancer sometimes is expressed as a probability, such as an odds ratio, percentage, or risk factor. The risk is based upon the presence or absence of one or more polymorphic variants described herein, and also may be based in part upon phenotypic traits of the individual being tested. Methods for calculating predispositions based upon patient data are well known (see, e.g., Agresti, Categorical Data Analysis, 2nd Ed. 2002. Wiley). Allelotyping and genotyping analyses may be carried out in populations other than those exemplified herein to enhance the predictive power of the prognostic method. These further analyses are executed in view of the exemplified procedures described herein, and may be based upon the same polymorphic variations or additional polymorphic variations. Risk determinations for breast cancer are useful in a variety of applications. In one embodiment, breast cancer risk determinations are used by clinicians to direct appropriate detection, preventative and treatment procedures to subjects who most require these. In another embodiment, breast cancer risk determinations are used by health insurers for preparing actuarial tables and for calculating insurance premiums.

The nucleic acid sample typically is isolated from a biological sample obtained from a subject. For example, nucleic acid can be isolated from blood, saliva, sputum, urine, cell scrapings, and biopsy tissue. The nucleic acid sample can be isolated from a biological sample using standard techniques, such as the technique described in Example 2. As used herein, the term “subject” refers primarily to humans but also refers to other mammals such as dogs, cats, and ungulates (e.g., cattle, sheep, and swine). Subjects also include avians (e.g., chickens and turkeys), reptiles, and fish (e.g., salmon), as embodiments described herein can be adapted to nucleic acid samples isolated from any of these organisms. The nucleic acid sample may be isolated from the subject and then directly utilized in a method for determining the presence of a polymorphic variant, or alternatively, the sample may be isolated and then stored (e.g., frozen) for a period of time before being subjected to analysis.

The presence or absence of a polymorphic variant is determined using one or both chromosomal complements represented in the nucleic acid sample. Determining the presence or absence of a polymorphic variant in both chromosomal complements represented in a nucleic acid sample from a subject having a copy of each chromosome is useful for determining the zygosity of an individual for the polymorphic variant (i.e., whether the individual is homozygous or heterozygous for the polymorphic variant). Any oligonucleotide-based diagnostic may be utilized to determine whether a sample includes the presence or absence of a polymorphic variant in a sample. For example, primer extension methods, ligase sequence determination methods (e.g., U.S. Pat. Nos. 5,679,524 and 5,952,174, and WO 01/27326), mismatch sequence determination methods (e.g., U.S. Pat. Nos. 5,851,770; 5,958,692; 6,110,684; and 6,183,958), microarray sequence determination methods, restriction fragment length polymorphism (RFLP), single strand conformation polymorphism detection (SSCP) (e.g., U.S. Pat. Nos. 5,891,625 and 6,013,499), PCR-based assays (e.g., TAQMAN® PCR System (Applied Biosystems)), and nucleotide sequencing methods may be used.

Oligonucleotide extension methods typically involve providing a pair of oligonucleotide primers in a polymerase chain reaction (PCR) or in other nucleic acid amplification methods for the purpose of amplifying a region from the nucleic acid sample that comprises the polymorphic variation. One oligonucleotide primer is complementary to a region 3′ of the polymorphism and the other is complementary to a region 5′ of the polymorphism. A PCR primer pair may be used in methods disclosed in U.S. Pat. Nos. 4,683,195; 4,683,202, 4,965,188; 5,656,493; 5,998,143; 6,140,054; WO 01/27327; and WO 01/27329 for example. PCR primer pairs may also be used in any commercially available machines that perform PCR, such as any of the GENEAMP® Systems available from Applied Biosystems. Also, those of ordinary skill in the art will be able to design oligonucleotide primers based upon a nucleotide sequence set forth herein without undue experimentation using knowledge readily available in the art.

Also provided is an extension oligonucleotide that hybridizes to the amplified fragment adjacent to the polymorphic variation. As used herein, the term “adjacent” refers to the 3′ end of the extension oligonucleotide being often 1 nucleotide from the 5′ end of the polymorphic site, and sometimes 2, 3, 4, 5, 6, 7, 8, 9, or 10 nucleotides from the 5′ end of the polymorphic site, in the nucleic acid when the extension oligonucleotide is hybridized to the nucleic acid. The extension oligonucleotide then is extended by one or more nucleotides, and the number and/or type of nucleotides that are added to the extension oligonucleotide determine whether the polymorphic variant is present. Oligonucleotide extension methods are disclosed, for example, in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; and WO 01/20039. Oligonucleotide extension methods using mass spectrometry are described, for example, in U.S. Pat. Nos. 5,547,835; 5,605,798; 5,691,141; 5,849,542; 5,869,242; 5,928,906; 6,043,031; and 6,194,144, and a method often utilized is described herein in Example 2. Multiple extension oligonucleotides may be utilized in one reaction, which is referred to herein as “multiplexing.”

A microarray can be utilized for determining whether a polymorphic variant is present or absent in a nucleic acid sample. A microarray may include any oligonucleotides described herein, and methods for making and using oligonucleotide microarrays suitable for diagnostic use are disclosed in U.S. Pat. Nos. 5,492,806; 5,525,464; 5,589,330; 5,695,940; 5,849,483; 6,018,041; 6,045,996; 6,136,541; 6,142,681; 6,156,501; 6,197,506; 6,223,127; 6,225,625; 6,229,911; 6,239,273; WO 00/52625; WO 01/25485; and WO 01/29259. The microarray typically comprises a solid support and the oligonucleotides may be linked to this solid support by covalent bonds or by non-covalent interactions. The oligonucleotides may also be linked to the solid support directly or by a spacer molecule. A microarray may comprise one or more oligonucleotides complementary to a polymorphic site shown in SEQ ID NO: 1 or below.

A kit also may be utilized for determining whether a polymorphic variant is present or absent in a nucleic acid sample. A kit often comprises one or more pairs of oligonucleotide primers useful for amplifying a fragment of a KIAA0861 nucleotide sequence or a substantially identical sequence thereof, where the fragment includes a polymorphic site. The kit sometimes comprises a polymerizing agent, for example, a thermostable nucleic acid polymerase such as one disclosed in U.S. Pat. No. 4,889,818 or 6,077,664. Also, the kit often comprises an elongation oligonucleotide that hybridizes to a KIAA0861 nucleotide sequence in a nucleic acid sample adjacent to the polymorphic site. Where the kit includes an elongation oligonucleotide, it also often comprises chain elongating nucleotides, such as DATP, dTTP, dGTP, dCTP, and dITP, including analogs of dATP, dTTP, dGTP, dCTP and dITP, provided that such analogs are substrates for a thermostable nucleic acid polymerase and can be incorporated into a nucleic acid chain elongated from the extension oligonucleotide. Along with chain elongating nucleotides would be one or more chain terminating nucleotides such as ddATP, ddTTP, ddGTP, ddCTP, and the like. In an embodiment, the kit comprises one or more oligonucleotide primer pairs, a polymerizing agent, chain elongating nucleotides, at least one elongation oligonucleotide, and one or more chain terminating nucleotides. Kits optionally include buffers, vials, microtiter plates, and instructions for use.

An individual identified as being at risk of breast cancer may be heterozygous or homozygous with respect to the allele associated with a higher risk of breast cancer. A subject homozygous for an allele associated with an increased risk of breast cancer is at a comparatively high risk of breast cancer, a subject heterozygous for an allele associated with an increased risk of breast cancer is at a comparatively intermediate risk of breast cancer, and a subject homozygous for an allele associated with a decreased risk of breast cancer is at a comparatively low risk of breast cancer. A genotype may be assessed for a complementary strand, such that the complementary nucleotide at a particular position is detected.

Also featured are methods for determining risk of breast cancer and/or identifying a subject at risk of breast cancer by contacting a polypeptide or protein encoded by a KIAA0861 nucleotide sequence from a subject with an antibody that specifically binds to an epitope associated with increased risk of breast cancer in the polypeptide. In certain embodiments, the antibody specifically binds to an epitope that comprises a leucine at amino acid position 359 in SEQ ID NO: 5, a leucine at amino acid position 378 in SEQ ID NO: 5, or an alanine at amino acid position 857 in SEQ ID NO: 5.

Applications of Prognostic and Diagnostic Results to Pharmacogenomic Methods

Pharmacogenomics is a discipline that involves tailoring a treatment for a subject according to the subject's genotype. For example, based upon the outcome of a prognostic test described herein, a clinician or physician may target pertinent information and preventative or therapeutic treatments to a subject who would be benefited by the information or treatment and avoid directing such information and treatments to a subject who would not be benefited (e.g., the treatment has no therapeutic effect and/or the subject experiences adverse side effects). As therapeutic approaches for breast cancer continue to evolve and improve, the goal of treatments for breast cancer related disorders is to intervene even before clinical signs (e.g., identification of lump in the breast) first manifest. Thus, genetic markers associated with susceptibility to breast cancer prove useful for early diagnosis, prevention and treatment of breast cancer.

The following is an example of a pharmacogenomic embodiment. A particular treatment regimen can exert a differential effect depending upon the subject's genotype. Where a candidate therapeutic exhibits a significant interaction with a major allele and a comparatively weak interaction with a minor allele (e.g., an order of magnitude or greater difference in the interaction), such a therapeutic typically would not be administered to a subject genotyped as being homozygous for the minor allele, and sometimes not administered to a subject genotyped as being heterozygous for the minor allele. In another example, where a candidate therapeutic is not significantly toxic when administered to subjects who are homozygous for a major allele but is comparatively toxic when administered to subjects heterozygous or homozygous for a minor allele, the candidate therapeutic is not typically administered to subjects who are genotyped as being heterozygous or homozygous with respect to the minor allele.

The methods described herein are applicable to pharmacogenomic methods for detecting, preventing, alleviating and/or treating breast cancer. For example, a nucleic acid sample from an individual may be subjected to a genetic test described herein. Where one or more polymorphic variations associated with increased risk of breast cancer are identified in a subject, information for detecting, preventing or treating breast cancer and/or one or more breast cancer detection, prevention and/or treatment regimens then may be directed to and/or prescribed to that subject.

In certain embodiments, a detection, prevenative and/or treatment regimen is specifically prescribed and/or administered to individuals who will most benefit from it based upon their risk of developing breast cancer assessed by the methods described herein. Thus, provided are methods for identifying a subject at risk of breast cancer and then prescribing a detection, therapeutic or preventative regimen to individuals identified as being at risk of breast cancer. Thus, certain embodiments are directed to methods for treating breast cancer in a subject, reducing risk of breast cancer in a subject, or early detection of breast cancer in a subject, which comprise: detecting the presence or absence of a polymorphic variant associated with breast cancer in a nucleotide sequence set forth in SEQ ID NO: 1 in a nucleic acid sample from a subject, where the nucleotide sequence comprises a polynucleotide sequence selected from the group consisting of: (a) a nucleotide sequence set forth in SEQ ID NO: 1; (b) a nucleotide sequence which encodes a polypeptide having an amino acid sequence encoded by a nucleotide sequence in SEQ ID NO: 1; (c) a nucleotide sequence which encodes a polypeptide that is 90% or more identical to an amino acid sequence encoded by a nucleotide sequence in SEQ ID NO: 1 or a nucleotide sequence about 90% or more identical to the nucleotide sequence set forth in SEQ ID NO: 1; and (d) a fragment of a nucleotide sequence of (a), (b), or (c), sometimes comprising a polymorphic site associated with breast cancer; and prescribing or administering a breast cancer treatment regimen, preventative regimen and/or detection regimen to a subject from whom the sample originated where the presence of one or more polymorphic variations associated with breast cancer are detected in the nucleotide sequence. In certain embodiments, one or more of the polymorphic variants described herein is detected. In these methods, genetic results may be utilized in combination with other test results to diagnose breast cancer as described above. Other test results include but are not limited to mammography results, imaging results, biopsy results and results from BRCA1 or BRAC2 test results, as described above.

Detection regimens include one or more mammography procedures, a regular mammography regimen (e.g., once a year, or once every six, four, three or two months); an early mammography regimen (e.g., mammography tests are performed beginning at age 25, 30, or 35); one or more biopsy procedures (e.g., a regular biopsy regimen beginning at age 40); breast biopsy and biopsy from other tissue; breast ultrasound and optionally ultrasound analysis of another tissue; breast magnetic resonance imaging (MRI) and optionally MRI analysis of another tissue; electrical impedance (T-scan) analysis of breast and optionally another tissue; ductal lavage; nuclear medicine analysis (e.g., scintimammography); BRCA1 and/or BRCA2 sequence analysis results; and/or thermal imaging of the breast and optionally another tissue.

Treatments sometimes are preventative (e.g., is prescribed or administered to reduce the probability that a breast cancer associated condition arises or progresses), sometimes are therapeutic, and sometimes delay, alleviate or halt the progression of breast cancer. Any known preventative or therapeutic treatment for alleviating or preventing the occurrence of breast cancer is prescribed and/or administered. For example, certain preventative treatments often are prescribed to subjects having a predisposition to breast cancer and where the subject is not diagnosed with breast cancer or is diagnosed as having symptoms indicative of early stage breast cancer (e.g., stage I). For subjects not diagnosed as having breast cancer, any preventative treatments known in the art can be prescribed and administered, which include selective hormone receptor modulators (e.g., selective estrogen receptor modulators (SERMs) such as tamoxifen, reloxifene, and toremifene); compositions that prevent production of hormones (e.g., aramotase inhibitors that prevent the production of estrogen in the adrenal gland, such as exemestane, letrozole, anastrozol, groserelin, and megestrol); other hormonal treatments (e.g., goserelin acetate and fulvestrant); biologic response modifiers such as antibodies (e.g., trastuzumab (herceptin/HER2)); anthracycline antibiotics (e.g., ellence/Pharmorubicin®); surgery (e.g., lumpectomy and mastectomy); drugs that delay or halt metastasis (e.g., pamidronate disodium); and alternative/complementary medicine (e.g., acupuncture, acupressure, moxibustion, qi gong, reiki, ayurveda, vitamins, minerals, and herbs (e.g., astragalus root, burdock root, garlic, green tea, and licorice root)).

The use of breast cancer treatments are well known in the art, and include surgery, chemotherapy and/or radiation therapy. Any of the treatments may be used in combination to treat or prevent breast cancer (e.g., surgery followed by radiation therapy or chemotherapy). Examples of chemotherapeutics are taxanes (e.g., docetaxel or paclitaxel), and examples of chemotherapy combinations used to treat breast cancer include: cyclophosphamide (Cytoxan), methotrexate (Amethopterin, Mexate, Folex), and fluorouracil (Fluorouracil, 5-Fu, Adrucil), which is referred to as CMF; cyclophosphamide, doxorubicin (Adriamycin), and fluorouracil, which is referred to as CAF; and doxorubicin (Adriamycin) and cyclophosphamide, which is referred to as AC.

As breast cancer preventative and treatment information can be specifically targeted to subjects in need thereof (e.g., those at risk of developing breast cancer or those that have early signs of breast cancer), provided herein is a method for preventing or reducing the risk of developing breast cancer in a subject, which comprises: (a) detecting the presence or absence of a polymorphic variation associated with breast cancer at a polymorphic site in a nucleotide sequence in a nucleic acid sample from a subject; (b) identifying a subject with a predisposition to breast cancer, whereby the presence of the polymorphic variation is indicative of a predisposition to breast cancer in the subject; and (c) if such a predisposition is identified, providing the subject with information about methods or products to prevent or reduce breast cancer or to delay the onset of breast cancer. Also provided is a method of targeting information or advertising to a subpopulation of a human population based on the subpopulation being genetically predisposed to a disease or condition, which comprises: (a) detecting the presence or absence of a polymorphic variation associated with breast cancer at a polymorphic site in a nucleotide sequence in a nucleic acid sample from a subject; (b) identifying the subpopulation of subjects in which the polymorphic variation is associated with breast cancer; and (c) providing information only to the subpopulation of subjects about a particular product which may be obtained and consumed or applied by the subject to help prevent or delay onset of the disease or condition.

Pharmacogenomics methods also may be used to analyze and predict a response to a breast cancer treatment or a drug. For example, if pharmacogenomics analysis indicates a likelihood that an individual will respond positively to a breast cancer treatment with a particular drug, the drug may be administered to the individual. Conversely, if the analysis indicates that an individual is likely to respond negatively to treatment with a particular drug, an alternative course of treatment may be prescribed. A negative response may be defined as either the absence of an efficacious response or the presence of toxic side effects. The response to a therapeutic treatment can be predicted in a background study in which subjects in any of the following populations are genotyped: a population that responds favorably to a treatment regimen, a population that does not respond significantly to a treatment regimen, and a population that responds adversely to a treatment regiment (e.g., exhibits one or more side effects). These populations are provided as examples and other populations and subpopulations may be analyzed. Based upon the results of these analyses, a subject is genotyped to predict whether he or she will respond favorably to a treatment regimen, not respond significantly to a treatment regimen, or respond adversely to a treatment regimen.

The methods described herein also are applicable to clinical drug trials. One or more polymorphic variants indicative of response to an agent for treating breast cancer or to side effects to an agent for treating breast cancer may be identified using the methods described herein. Thereafter, potential participants in clinical trials of such an agent may be screened to identify those individuals most likely to respond favorably to the drug and exclude those likely to experience side effects. In that way, the effectiveness of drug treatment may be measured in individuals who respond positively to the drug, without lowering the measurement as a result of the inclusion of individuals who are unlikely to respond positively in the study and without risking undesirable safety problems. In certain embodiments, the agent for treating breast cancer described herein targets KIAA0861 or a target in the KIAA0861 pathway (e.g., Rho GTPase).

Thus, another embodiment is a method of selecting an individual for inclusion in a clinical trial of a treatment or drug comprising the steps of: (a) obtaining a nucleic acid sample from an individual; (b) determining the identity of a polymorphic variation which is associated with a positive response to the treatment or the drug, or at least one polymorphic variation which is associated with a negative response to the treatment or the drug in the nucleic acid sample, and (c) including the individual in the clinical trial if the nucleic acid sample contains said polymorphic variation associated with a positive response to the treatment or the drug or if the nucleic acid sample lacks said polymorphic variation associated with a negative response to the treatment or the drug. In addition, the methods for selecting an individual for inclusion in a clinical trial of a treatment or drug encompass methods with any further limitation described in this disclosure, or those following, specified alone or in any combination. The polymorphic variation may be in a sequence selected individually or in any combination from the group consisting of (i) a polynucleotide sequence set forth in SEQ ID NO: 1; (ii) a polynucleotide sequence that is 90% or more identical to a nucleotide sequence set forth in SEQ ID NO: 1; (iii) a polynucleotide sequence that encodes a polypeptide having an amino acid sequence identical to or 90% or more identical to an amino acid sequence encoded by a nucleotide sequence set forth in SEQ ID NO: 1; and (iv) a fragment of a polynucleotide sequence of (i), (ii), or (iii) comprising the polymorphic site. The including step (c) optionally comprises administering the drug or the treatment to the individual if the nucleic acid sample contains the polymorphic variation associated with a positive response to the treatment or the drug and the nucleic acid sample lacks said biallelic marker associated with a negative response to the treatment or the drug.

Also provided herein is a method of partnering between a diagnostic/prognostic testing provider and a provider of a consumable product, which comprises: (a) the diagnostic/prognostic testing provider detects the presence or absence of a polymorphic variation associated with breast cancer at a polymorphic site in a nucleotide sequence in a nucleic acid sample from a subject; (b) the diagnostic/prognostic testing provider identifies the subpopulation of subjects in which the polymorphic variation is associated with breast cancer; (c) the diagnostic/prognostic testing provider forwards information to the subpopulation of subjects about a particular product which may be obtained and consumed or applied by the subject to help prevent or delay onset of the disease or condition; and (d) the provider of a consumable product forwards to the diagnostic test provider a fee every time the diagnostic/prognostic test provider forwards information to the subject as set forth in step (c) above.

Compositions Comprising Breast Cancer-Directed Molecules

Featured herein is a composition comprising a breast cancer cell and one or more molecules specifically directed and targeted to a nucleic acid comprising a KIAA0861 nucleotide sequence or a KIAA0861 polypeptide. Such directed molecules include, but are not limited to, a compound that binds to a KIAA0861 nucleic acid or a KIAA0861 polypeptide; a RNAi or siRNA molecule having a strand complementary to a KIAA0861 nucleotide sequence; an antisense nucleic acid complementary to an RNA encoded by a KIAA0861 DNA sequence; a ribozyme that hybridizes to a KIAA0861 nucleotide sequence; a nucleic acid aptamer that specifically binds a KIAA0861 polypeptide; and an antibody that specifically binds to a KIAA0861 polypeptide or binds to a KIAA0861 nucleic acid. In certain embodiments, the antibody specifically binds to an epitope that comprises a leucine at amino acid position 359 in SEQ ID NO: 5, a leucine at amino acid position 378 in SEQ ID NO: 5, or an alanine at amino acid position 857 in SEQ ID NO: 5. In specific embodiments, the breast cancer directed molecule interacts with a KIAA0861 nucleic acid or polypeptide variant associated with breast cancer. In other embodiments, the breast cancer directed molecule interacts with a polypeptide involved in the KIAA0861 signal pathway, or a nucleic acid encoding such a polypeptide. Polypeptides involved in the KIAA0861 signal pathway are discussed herein.

Compositions sometimes include an adjuvant known to stimulate an immune response, and in certain embodiments, an adjuvant that stimulates a T-cell lymphocyte response. Adjuvants are known, including but not limited to an aluminum adjuvant (e.g., aluminum hydroxide); a cytokine adjuvant or adjuvant that stimulates a cytokine response (e.g., interleukin (IL)-12 and/or γ-interferon cytokines); a Freund-type mineral oil adjuvant emulsion (e.g., Freund's complete or incomplete adjuvant); a synthetic lipoid compound; a copolymer adjuvant (e.g., TitreMax); a saponin; Quil A; a liposome; an oil-in-water emulsion (e.g., an emulsion stabilized by Tween 80 and pluronic polyoxyethlene/polyoxypropylene block copolymer (Syntex Adjuvant Formulation); TitreMax; detoxified endotoxin (MPL) and mycobacterial cell wall components (TDW, CWS) in 2% squalene (Ribi Adjuvant System)); a muramyl dipeptide; an immune-stimulating complex (ISCOM, e.g., an Ag-modified saponin/cholesterol micelle that forms stable cage-like structure); an aqueous phase adjuvant that does not have a depot effect (e.g., Gerbu adjuvant); a carbohydrate polymer (e.g., AdjuPrime); L-tyrosine; a manide-oleate compound (e.g., Montanide); an ethylene-vinyl acetate copolymer (e.g., Elvax 40W1,2); or lipid A, for example. Such compositions are useful for generating an immune response against a breast cancer directed molecule (e.g., an HLA-binding subsequence within a polypeptide encoded by a nucleotide sequence in SEQ ID NO: 1). In such methods, a peptide having an amino acid subsequence of a polypeptide encoded by a nucleotide sequence in SEQ ID NO: 1 is delivered to a subject, where the subsequence binds to an HLA molecule and induces a CTL lymphocyte response. The peptide sometimes is delivered to the subject as an isolated peptide or as a minigene in a plasmid that encodes the peptide. Methods for identifying HLA-binding subsequences in such polypeptides are known (see e.g., publication WO02/20616 and PCT application US98/01373 for methods of identifying such sequences).

The breast cancer cell may be in a group of breast cancer cells and/or other types of cells cultured in vitro or in a tissue having breast cancer cells (e.g., a melanocytic lesion) maintained in vitro or present in an animal in vivo (e.g., a rat, mouse, ape or human). In certain embodiments, a composition comprises a component from a breast cancer cell or from a subject having a breast cancer cell instead of the breast cancer cell or in addition to the breast cancer cell, where the component sometimes is a nucleic acid molecule (e.g., genomic DNA), a protein mixture or isolated protein, for example. The aforementioned compositions have utility in diagnostic, prognostic and pharmacogenomic methods described previously and in breast cancer therapeutics described hereafter. Certain breast cancer molecules are described in greater detail below.

Compounds

Compounds can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive (see, e.g., Zuckermann et al, J. Med. Chem. 37: 2678-85 (1994)); spatially addressable parallel solid phase or solution phase libraries; synthetic library methods requiring deconvolution; “one-bead one-compound” library methods; and synthetic library methods using affinity chromatography selection. Biological library and peptoid library approaches are typically limited to peptide libraries, while the other approaches are applicable to peptide, non-peptide oligomer or small molecule libraries of compounds (Lam, Anticancer Drug Des. 12: 145, (1997)). Examples of methods for synthesizing molecular libraries are described, for example, in DeWitt et al., Proc. Natl. Acad. Sci. U.S.A. 90: 6909 (1993); Erb et al, Proc. Natl. Acad. Sci. USA 91: 11422 (1994); Zuckermann et al., J. Med. Chem. 37: 2678 (1994); Cho et al., Science 261: 1303 (1993); Carrell et al., Angew. Chem. Int. Ed. Engl. 33: 2059 (1994); Carell et al., Angew. Chem. Int. Ed. Engl. 33: 2061 (1994); and in Gallop et al., J. Med. Chem. 37: 1233 (1994).

Libraries of compounds may be presented in solution (e.g., Houghten, Biotechniques 13: 412-421 (1992)), or on beads (Lam, Nature 354: 82-84 (1991)), chips (Fodor, Nature 364: 555-556 (1993)), bacteria or spores (Ladner, U.S. Pat. No. 5,223,409), plasmids (Cull et al., Proc. Natl. Acad. Sci. USA 89: 1865-1869 (1992)) or on phage (Scott and Smith, Science 249: 386-390 (1990); Devlin, Science 249: 404-406 (1990); Cwirla et al., Proc. Natl. Acad. Sci. 87: 6378-6382 (1990); Felici, J. Mol. Biol. 222: 301-310 (1991); Ladner supra.).

A compound sometimes alters expression and sometimes alters activity of a KIAA0861 polypeptide and may be a small molecule. Small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Antisense Nucleic Acid Molecules, Ribozymes, RNAi, siRNA and Modified Nucleic Acid Molecules

An “antisense” nucleic acid refers to a nucleotide sequence complementary to a “sense” nucleic acid encoding a polypeptide, e.g., complementary to the coding strand of a double-stranded cDNA molecule or complementary to an mRNA sequence. The antisense nucleic acid can be complementary to an entire coding strand in SEQ ID NO: 1, 2 or 3, or to a portion thereof or a substantially identical sequence thereof. In another embodiment, the antisense nucleic acid molecule is antisense to a “noncoding region” of the coding strand of a nucleotide sequence in SEQ ID NO: 1 (e.g., 5′ and 3′ untranslated regions).

An antisense nucleic acid can be designed such that it is complementary to the entire coding region of an mRNA encoded by a nucleotide sequence in SEQ ID NO: 1 (e.g., SEQ ID NO: 2 or 3), and often the antisense nucleic acid is an oligonucleotide antisense to only a portion of a coding or noncoding region of the mRNA. For example, the antisense oligonucleotide can be complementary to the region surrounding the translation start site of the mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length. The antisense nucleic acids, which include the ribozymes described hereafter, can be designed to target a nucleotide sequence in SEQ ID NO: 1, often a variant associated with breast cancer, or a substantially identical sequence thereof. Among the variants, minor alleles and major alleles can be targeted, and those associated with a higher risk of breast cancer are often designed, tested, and administered to subjects.

An antisense nucleic acid can be constructed using chemical synthesis and enzymatic ligation reactions using standard procedures. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

When utilized as therapeutics, antisense nucleic acids typically are administered to a subject (e.g., by direct injection at a tissue site) or generated in situ such that they hybridize with or bind to cellular mRNA and/or genomic DNA encoding a polypeptide and thereby inhibit expression of the polypeptide, for example, by inhibiting transcription and/or translation. Alternatively, antisense nucleic acid molecules can be modified to target selected cells and then are administered systemically. For systemic administration, antisense molecules can be modified such that they specifically bind to receptors or antigens expressed on a selected cell surface, for example, by linking antisense nucleic acid molecules to peptides or antibodies which bind to cell surface receptors or antigens. Antisense nucleic acid molecules can also be delivered to cells using the vectors described herein. Sufficient intracellular concentrations of antisense molecules are achieved by incorporating a strong promoter, such as a pol II or pol III promoter, in the vector construct.

Antisense nucleic acid molecules sometimes are alpha-anomeric nucleic acid molecules. An alpha-anomeric nucleic acid molecule forms specific double-stranded hybrids with complementary RNA in which, contrary to the usual beta-units, the strands run parallel to each other (Gaultier et al., Nucleic Acids. Res. 15: 6625-6641 (1987)). Antisense nucleic acid molecules can also comprise a 2′-o-methylribonucleotide (Inoue et al., Nucleic Acids Res. 15: 6131-6148 (1987)) or a chimeric RNA-DNA analogue (Inoue et al., FEBS Lett. 215: 327-330 (1987)). Antisense nucleic acids sometimes are composed of DNA or PNA or any other nucleic acid derivatives described previously.

In another embodiment, an antisense nucleic acid is a ribozyme. A ribozyme having specificity for a KIAA0861 nucleotide sequence can include one or more sequences complementary to such a nucleotide sequence, and a sequence having a known catalytic region responsible for mRNA cleavage (see e.g., U.S. Pat. No. 5,093,246 or Haselhoff and Gerlach, Nature 334: 585-591 (1988)). For example, a derivative of a Tetrahymena L-19 IVS RNA is sometimes utilized in which the nucleotide sequence of the active site is complementary to the nucleotide sequence to be cleaved in a mRNA (see e.g., Cech et al. U.S. Pat. No. 4,987,071; and Cech et al. U.S. Pat. No. 5,116,742). Also, target mRNA sequences can be used to select a catalytic RNA having a specific ribonuclease activity from a pool of RNA molecules (see e.g., Bartel & Szostak, Science 261: 1411-1418 (1993)).

Breast cancer directed molecules include in certain embodiments nucleic acids that can form triple helix structures with a KIAA0861 nucleotide sequence or a substantially identical sequence thereof, especially one that includes a regulatory region that controls expression of a polypeptide. Gene expression can be inhibited by targeting nucleotide sequences complementary to the regulatory region of a KIAA0861 nucleotide sequence or a substantially identical sequence (e.g., promoter and/or enhancers) to form triple helical structures that prevent transcription of a gene in target cells (see e.g., Helene, Anticancer Drug Des. 6(6): 569-84 (1991); Helene et al., Ann. N.Y. Acad. Sci. 660: 27-36 (1992); and Maher, Bioassays 14(12): 807-15 (1992). Potential sequences that can be targeted for triple helix formation can be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

Breast cancer directed molecules include RNAi and siRNA nucleic acids. Gene expression may be inhibited by the introduction of double-stranded RNA (dsRNA), which induces potent and specific gene silencing, a phenomenon called RNA interference or RNAi. See, e.g., Fire et al., U.S. Pat. No. 6,506,559; Tuschl et al. PCT International Publication No. WO 01/75164; Kay et al. PCT International Publication No. WO 03/010180A1; or Bosher J M, Labouesse, Nat Cell Biol 2000 February; 2(2):E31-6. This process has been improved by decreasing the size of the double-stranded RNA to 20-24 base pairs (to create small-interfering RNAs or siRNAs) that “switched off” genes in mammalian cells without initiating an acute phase response, i.e., a host defense mechanism that often results in cell death (see, e.g., Caplen et al. Proc Natl Acad Sci USA. 2001 Aug. 14; 98(17):9742-7 and Elbashir et al. Methods 2002 February; 26(2):199-213). There is increasing evidence of post-transcriptional gene silencing by RNA interference (RNAi) for inhibiting targeted expression in mammalian cells at the mRNA level, in human cells. There is additional evidence of effective methods for inhibiting the proliferation and migration of tumor cells in human patients, and for inhibiting metastatic cancer development (see, e.g., U.S. Patent Application No. US2001000993183; Caplen et al. Proc Natl Acad Sci USA; and Abderrahmani et al. Mol Cell Biol 2001 Nov. 21 (21):7256-67).

An “siRNA” or “RNAi” refers to a nucleic acid that forms a double stranded RNA and has the ability to reduce or inhibit expression of a gene or target gene when the siRNA is delivered to or expressed in the same cell as the gene or target gene. “siRNA” refers to short double-stranded RNA formed by the complementary strands. Complementary portions of the siRNA that hybridize to form the double stranded molecule often have substantial or complete identity to the target molecule sequence. In one embodiment, an siRNA refers to a nucleic acid that has substantial or complete identity to a target gene and forms a double stranded siRNA.

When designing the siRNA molecules, the targeted region often is selected from a given DNA sequence beginning 50 to 100 nucleotides downstream of the start codon. See, e.g., Elbashir et al., Methods 26:199-213 (2002). Initially, 5′ or 3′ UTRs and regions nearby the start codon were avoided assuming that UTR-binding proteins and/or translation initiation complexes may interfere with binding of the siRNP or RISC endonuclease complex. Sometimes regions of the target 23 nucleotides in length conforming to the sequence motif AA(N19)TT (SEQ ID NO: 6) (N, an nucleotide), and regions with approximately 30% to 70% G/C-content (often about 50% G/C-content) often are selected. If no suitable sequences are found, the search often is extended using the motif NA(N21). The sequence of the sense siRNA sometimes corresponds to (N19) TT or N21 (position 3 to 23 of the 23-nt motif), respectively. In the latter case, the 3′ end of the sense siRNA often is converted to TT. The rationale for this sequence conversion is to generate a symmetric duplex with respect to the sequence composition of the sense and antisense 3′ overhangs. The antisense siRNA is synthesized as the complement to position 1 to 21 of the 23-nt motif. Because position 1 of the 23-nt motif is not recognized sequence-specifically by the antisense siRNA, the 3′-most nucleotide residue of the antisense siRNA can be chosen deliberately. However, the penultimate nucleotide of the antisense siRNA (complementary to position 2 of the 23-nt motif) often is complementary to the targeted sequence. For simplifying chemical synthesis, TT often is utilized. siRNAs corresponding to the target motif NAR(N17)YNN, where R is purine (A,G) and Y is pyrimidine (C,U), often are selected. Respective 21 nucleotide sense and antisense siRNAs often begin with a purine nucleotide and can also be expressed from pol III expression vectors without a change in targeting site. Expression of RNAs from pol III promoters often is efficient when the first transcribed nucleotide is a purine.

The sequence of the siRNA can correspond to the full length target gene, or a subsequence thereof. Often, the siRNA is about 15 to about 50 nucleotides in length (e.g., each complementary sequence of the double stranded siRNA is 15-50 nucleotides in length, and the double stranded siRNA is about 15-50 base pairs in length, sometimes about 20-30 nucleotides in length or about 20-25 nucleotides in length, e.g., 20, 21, 22, 23, 24, 25, 26, 27, 28, 29, or 30 nucleotides in length. The siRNA sometimes is about 21 nucleotides in length. Methods of using siRNA are well known in the art, and specific siRNA molecules may be purchased from a number of companies including Dharmacon Research, Inc.

Antisense, ribozyme, RNAi and siRNA nucleic acids can be altered to form modified nucleic acid molecules. The nucleic acids can be altered at base moieties, sugar moieties or phosphate backbone moieties to improve stability, hybridization, or solubility of the molecule. For example, the deoxyribose phosphate backbone of nucleic acid molecules can be modified to generate peptide nucleic acids (see Hyrup et al., Bioorganic & Medicinal Chemistry 4 (1): 5-23 (1996)). As used herein, the terms “peptide nucleic acid” or “PNA” refers to a nucleic acid mimic such as a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. Synthesis of PNA oligomers can be performed using standard solid phase peptide synthesis protocols as described, for example, in Hyrup et al, (1996) supra and Perry-O'Keefe et al, Proc. Natl. Acad. Sci. 93: 14670-675 (1996).

PNA nucleic acids can be used in prognostic, diagnostic, and therapeutic applications. For example, PNAs can be used as antisense or antigene agents for sequence-specific modulation of gene expression by, for example, inducing transcription or translation arrest or inhibiting replication. PNA nucleic acid molecules can also be used in the analysis of single base pair mutations in a gene, (e.g., by PNA-directed PCR clamping); as “artificial restriction enzymes” when used in combination with other enzymes, (e.g., S1 nucleases (Hyrup (1996) supra)); or as probes or primers for DNA sequencing or hybridization (Hyrup et al, (1996) supra; Perry-O'Keefe supra).

In other embodiments, oligonucleotides may include other appended groups such as peptides (e.g., for targeting host cell receptors in vivo), or agents facilitating transport across cell membranes (see e.g., Letsinger et al., Proc. Natl. Acad. Sci. USA 86: 6553-6556 (1989); Lemaitre et al., Proc. Natl. Acad. Sci. USA 84: 648-652 (1987); PCT Publication No. WO88/09810) or the blood-brain barrier (see, e.g., PCT Publication No. WO89/10134). In addition, oligonucleotides can be modified with hybridization-triggered cleavage agents (See, e.g., Krol et al., Bio-Techniques 6: 958-976 (1988)) or intercalating agents. (See, e.g., Zon, Pharm. Res. 5: 539-549 (1988)). To this end, the oligonucleotide may be conjugated to another molecule, (e.g., a peptide, hybridization triggered cross-linking agent, transport agent, or hybridization-triggered cleavage agent).

Also included herein are molecular beacon oligonucleotide primer and probe molecules having one or more regions complementary to a nucleotide sequence of SEQ ID NO: 1, 2, or 3 or a substantially identical sequence thereof, two complementary regions one having a fluorophore and one a quencher such that the molecular beacon is useful for quantifying the presence of the nucleic acid in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

Antibodies

The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments which can be generated by treating the antibody with an enzyme such as pepsin. An antibody sometimes is a polyclonal, monoclonal, recombinant (e.g., a chimeric or humanized), fully human, non-human (e.g., murine), or a single chain antibody. An antibody may have effector function and can fix complement, and is sometimes coupled to a toxin or imaging agent.

A full-length polypeptide or antigenic peptide fragment encoded by a KIAA0861 nucleotide sequence can be used as an immunogen or can be used to identify antibodies made with other immunogens, e.g., cells, membrane preparations, and the like. An antigenic peptide often includes at least 8 amino acid residues of the amino acid sequences encoded by a nucleotide sequence of SEQ ID NO: 1, 2 or 3, or substantially identical sequence thereof, and encompasses an epitope. Antigenic peptides sometimes include 10 or more amino acids, 15 or more amino acids, 20 or more amino acids, or 30 or more amino acids. Hydrophilic and hydrophobic fragments of polypeptides sometimes are used as immunogens.

Epitopes encompassed by the antigenic peptide are regions located on the surface of the polypeptide (e.g., hydrophilic regions) as well as regions with high antigenicity. For example, an Emini surface probability analysis of the human polypeptide sequence can be used to indicate the regions that have a particularly high probability of being localized to the surface of the polypeptide and are thus likely to constitute surface residues useful for targeting antibody production. The antibody may bind an epitope on any domain or region on polypeptides described herein.

Also, chimeric, humanized, and completely human antibodies are useful for applications which include repeated administration to subjects. Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al International Application No. PCT/US86/02269; Akira, et al European Patent Application 184,187; Taniguchi, M., European Patent Application 171,496; Morrison et al European Patent Application 173,494; Neuberger et al PCT International Publication No. WO 86/01533; Cabilly et al U.S. Pat. No. 4,816,567; Cabilly et al European Patent Application 125,023; Better et al, Science 240: 1041-1043 (1988); Liu et al., Proc. Natl. Acad. Sci. USA 84: 3439-3443 (1987); Liu et al., J. Immunol. 139: 3521-3526 (1987); Sun et al., Proc. Natl. Acad. Sci. USA 84: 214-218 (1987); Nishimura et al., Canc. Res. 47: 999-1005 (1987); Wood et al., Nature 314: 446-449 (1985); and Shaw et al., J. Natl. Cancer Inst. 80: 1553-1559 (1988); Morrison, S. L., Science 229: 1202-1207 (1985); Oi et al., BioTechniques 4: 214 (1986); Winter U.S. Pat. No. 5,225,539; Jones et al., Nature 321: 552-525 (1986); Verhoeyan et al., Science 239: 1534; and Beidler et al., J. Immunol. 141: 4053-4060 (1988).

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar, Int. Rev. Immunol. 13: 65-93 (1995); and U.S. Pat. Nos. 5,625,126; 5,633,425; 5,569,825; 5,661,016; and 5,545,806. In addition, companies such as Abgenix, Inc. (Fremont, Calif.) and Medarex, Inc. (Princeton, N.J.), can be engaged to provide human antibodies directed against a selected antigen using technology similar to that described above. Completely human antibodies that recognize a selected epitope also can be generated using a technique referred to as “guided selection.” In this approach a selected non-human monoclonal antibody (e.g., a murine antibody) is used to guide the selection of a completely human antibody recognizing the same epitope. This technology is described for example by Jespers et al., Bio/Technology 12: 899-903 (1994).

Antibody can be a single chain antibody. A single chain antibody (scFV) can be engineered (see, e.g., Colcher et al., Ann. N Y Acad. Sci. 880: 263-80 (1999); and Reiter, Clin. Cancer Res. 2: 245-52 (1996)). Single chain antibodies can be dimerized or multimerized to generate multivalent antibodies having specificities for different epitopes of the same target polypeptide.

Antibodies also may be selected or modified so that they exhibit reduced or no ability to bind an Fc receptor. For example, an antibody may be an isotype or subtype, fragment or other mutant, which does not support binding to an Fc receptor (e.g., it has a mutagenized or deleted Fc receptor binding region).

Also, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1 dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, melphalan, carmustine (BCNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Antibody conjugates can be used for modifying a given biological response. For example, the drug moiety may be a protein or polypeptide possessing a desired biological activity. Such proteins may include, for example, a toxin such as abrin, ricin A, pseudomonas exotoxin, or diphtheria toxin; a polypeptide such as tumor necrosis factor, γ-interferon, α-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Also, an antibody can be conjugated to a second antibody to form an antibody heteroconjugate as described by Segal in U.S. Pat. No. 4,676,980, for example.

An antibody (e.g., monoclonal antibody) can be used to isolate target polypeptides by standard techniques, such as affinity chromatography or immunoprecipitation. Moreover, an antibody can be used to detect a target polypeptide (e.g., in a cellular lysate or cell supernatant) in order to evaluate the abundance and pattern of expression of the polypeptide. Antibodies can be used diagnostically to monitor polypeptide levels in tissue as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H. Also, an antibody can be utilized as a test molecule for determining whether it can treat breast cancer, and as a therapeutic for administration to a subject for treating breast cancer.

An antibody can be made by immunizing with a purified antigen, or a fragment thereof, e.g., a fragment described herein, a membrane associated antigen, tissues, e.g., crude tissue preparations, whole cells, preferably living cells, lysed cells, or cell fractions.

Included herein are antibodies which bind only a native polypeptide, only denatured or otherwise non-native polypeptide, or which bind both, as well as those having linear or conformational epitopes. Conformational epitopes sometimes can be identified by selecting antibodies that bind to native but not denatured polypeptide. Also featured are antibodies that specifically bind to a polypeptide variant associated with breast cancer.

Screening Assays

Featured herein are methods for identifying a candidate therapeutic for treating breast cancer. The methods comprise contacting a test molecule with a target molecule in a system. A “target molecule” as used herein refers to a nucleic acid of SEQ ID NO: 1, 2 or 3, a substantially identical nucleic acid thereof, or a fragment thereof, and an encoded polypeptide of the foregoing. The method also comprises determining the presence or absence of an interaction between the test molecule and the target molecule, where the presence of an interaction between the test molecule and the nucleic acid or polypeptide identifies the test molecule as a candidate breast cancer therapeutic. The interaction between the test molecule and the target molecule may be quantified.

Test molecules and candidate therapeutics include, but are not limited to, compounds, antisense nucleic acids, siRNA molecules, ribozymes, polypeptides or proteins encoded by a KIAA0861 nucleic acids, or a substantially identical sequence or fragment thereof, and immunotherapeutics (e.g., antibodies and HLA-presented polypeptide fragments). A test molecule or candidate therapeutic may act as a modulator of target molecule concentration or target molecule function in a system. A “modulator” may agonize (i.e., up-regulates) or antagonize (i.e., down-regulates) a target molecule concentration partially or completely in a system by affecting such cellular functions as DNA replication and/or DNA processing (e.g., DNA methylation or DNA repair), RNA transcription and/or RNA processing (e.g., removal of intronic sequences and/or translocation of spliced mRNA from the nucleus), polypeptide production (e.g., translation of the polypeptide from mRNA), and/or polypeptide post-translational modification (e.g., glycosylation, phosphorylation, and proteolysis of pro-polypeptides). A modulator may also agonize or antagonize a biological function of a target molecule partially or completely, where the function may include adopting a certain structural conformation, interacting with one or more binding partners, ligand binding, catalysis (e.g., phosphorylation, dephosphorylation, hydrolysis, methylation, and isomerization), and an effect upon a cellular event (e.g., effecting progression of breast cancer).

As used herein, the term “system” refers to a cell free in vitro environment and a cell-based environment such as a collection of cells, a tissue, an organ, or an organism. A system is “contacted” with a test molecule in a variety of manners, including adding molecules in solution and allowing them to interact with one another by diffusion, cell injection, and any administration routes in an animal. As used herein, the term “interaction” refers to an effect of a test molecule on test molecule, where the effect sometimes is binding between the test molecule and the target molecule, and sometimes is an observable change in cells, tissue, or organism.

There are many standard methods for detecting the presence or absence of an interaction between a test molecule and a target molecule. For example, titrametric, acidimetric, radiometric, NMR, monolayer, polarographic, spectrophotometric, fluorescent, and ESR assays probative of a target molecule interaction may be utilized.

KIAA0861 activity and/or KIAA0861 interactions can be detected and quantified using assays known in the art. For example, an immunoprecipitation assay or a kinase activity assay that employs a kinase-inactivated MEK can be utilized. Kinase inactivated MEKs are known in the art, such as a MEK that includes the mutation K97M. In these assays, mammalian cells (e.g., COS or NIH-3T3) are transiently transfected with constructs expressing KIAA0861, and in addition, the cells are co-transfected with oncogenic RAS or SRC or both. Oncogenic RAS or SRC activates KIAA0861 kinase activity. KIAA0861 is immunoprecipitated from cell extracts using a monoclonal antibody (e.g., 9E10) or a polyclonal antibody (e.g., from rabbit) specific for a unique peptide from KIAA0861. KIAA0861 is then resuspended in assay buffer containing GST-Mekl or GST-Mek2 and/or GST-ERK2. In addition, [γ³²P] ATP can be added to detect and/or quantify phosphorylation activity. Samples are incubated for 5-30 minutes at 30° C., and then the reaction is terminated by addition of EDTA. The samples are centrifuged and the supernatant fractions are collected. Phosphorylation activity is detected using one of two methods: (i) activity of GST-ERK2 kinase can be measured using MBP (myelin basic protein, a substrate for ERK) as substrate, or (ii) following incubation of immunoprecipitated KIAA0861 in reaction buffer containing GST-ERK and [γ³²P] ATP, transfer of labeled ATP to kinase-dead ERK can be quantified by a phosphor-imager or densitometer following PAGE separation of polypeptide products (phosphorylated and non-phosphorylated forms). These types of assays are described in Weber et al., Oncogene 19: 169-176 (2000); Mason et al., EMBO J. 18: 2137-2148 (1999); Marais et al., J. Biol. Chem. 272: 4378-4383 (1997); Marais et al., EMBO J. 14: 3136-3145 (1995).

Screening assays also are performed to identify molecules that regulate the interaction between a GEF, such as KIAA0861, and a GTPase. Such molecules can be identified using an assay for a GEF activity, such as guanine nucleotide exchange activity, binding to a guanine nucleotide-depleted site of a GTPase, or oncogenic transforming activity, or a TGPase activity such as GTP hydrolysis. In general, a compound having such an in vitro activity will be useful in vivo to modulate a biological pathway associated with a GTPase (e.g., to treat a pathological condition associated with the biological and cellular activities mentioned above). By way of illustration, the ways in which GEF regulators can be identified are described above and below in terms of Rho and KIAA0861. However, it is to be understood that such methods can be applied generally to other GEFs.

A guanine nucleotide exchange assay, e.g., as described in Hart et al., Nature, 354:311-314, 28 Nov. 1991, can be used to assay for the ability of a compound to regulate the interaction between Rho and KIAA0861. For example, Rho protein (recombinant, recombinant fusion protein, or isolated from natural sources) is labeled with tritiated-GDP. The tritiated-GDP-labeled Rho is then incubated with KIAA0861 and GTP under conditions in which nucleotide exchange occurs. The amount of tritiated-GDP that is retained by Rho is determined by separating bound GDP from free GDP, e.g., using a BA85 filter. The ability of a compound to regulate the interaction can be determined by adding the compound at a desired time to the incubation (e.g., before addition of KIAA0861, after addition of KIAA0861) and determining its effect on nucleotide exchange. Various agonist and antagonists of the interaction can be identified in this manner.

Binding to a guanine nucleotide-depleted site of Rho can be determined in various ways, e.g., as described in Hart et al., J. Biol. Chem. 269:62-65, 1994. Briefly, a Rho protein can be coupled to a solid support using various methods that one skilled in the art would know, e.g., using an antibody to Rho, a fusion protein between Rho and a marker protein, such as glutathione protein (GST), wherein the fusion is coupled to a solid support via the marker protein (such as glutathionine beads when GST is used), and the like. The Rho protein is converted to a guanine nucleotide depleted state (for effective conditions, see, e.g., Hart et al., J. Biol. Chem., 269:62-65, 1994) and incubated with, e.g., GDP, GTP γS, and GEF such as KIAA0861. The solid support is then separated and any protein on it run on a gel. A compound can be added at any time during the incubation (as described above) to determine its effect on the binding of GEF to Rho.

Modulation of oncogenic transforming activity by a KIAA0861, or derivatives thereof can be measured according to various known procedures, e.g., Eva and Aaronson, Nature, 316:273-275, 1985; Hart et al., J. Biol. Chem., 269:62-65, 1994. A compound can be added at any time during the method (e.g., pretreatment of cells; after addition of GEF, and the like) to determine its effect on the oncogenic transforming activity of KIAA0861. Various cell lines also can be used.

Other assays for Rho-mediated signal transduction can be accomplished according in analogy to procedures known in the art, e.g., as described in U.S. Pat. Nos. 5,141,851; 5,420,334; 5,436,128; and 5,482,954; WO94/16069; WO93/16179; WO91/15582; WO90/00607. In addition, peptides which inhibit the interaction, e.g., binding between KIAA0861 and a G-protein, such as RhoA, can be identified and prepared according to EP 496 162.

Included herein are methods of testing for and identifying an agent which modulates the guanine nucleotide exchange activity of a guanine nucleotide exchange factor, or a biologically-active fragment thereof, or which modulates the binding between a GEF, or a biologically-active fragment thereof, and a GTPase, or a biologically-active fragment thereof, to which it binds. The method comprises contacting the GEF and GTPase with an agent to be tested and then detecting the presence or amount of binding between the GEF and GTPase, or an activity of the GEF such as guanine nucleotide exchange activity. As discussed herein “modulating” refers to an agent that affects the activity or binding of a GEF such as KIAA0861. The binding or activity modulation can be affected in various ways, including inhibiting, blocking, preventing, increasing, enhancing, or promoting it. The binding or activity affected does not have to be achieved in a specific way, e.g., it can be competitive, noncompetitive, allosteric, sterically hindered, via cross-linking between the agent and the GEF or GTPase, or the like. The agent can act on either the GEF or GTPase. The agent can be an agonist, an antagonist, or a partial agonist or antagonist. The presence or amount of binding can be determined in various ways, e.g., directly or indirectly by assaying for an activity promoted or inhibited by the GEF, such as guanine nucleotide exchange, GTP hydrolysis, oncogenic transformation, and the like. Such assays are described above and below, and are also known in the art. The agent can be obtained and/or prepared from a variety of sources, including natural and synthetic. It can comprise, e.g., amino acids, lipids, carbohydrates, organic molecules, nucleic acids, inorganic molecules, or mixtures thereof. See, e.g., Hoeprich, Nature Biotechnology, 14:1311-1312, 1996, which describes an example of automated synthesis of organic molecules. The agent can be added simultaneously or sequentially. For example, the agent can be added to the GEF and then the resultant mixture can be further combined with the GTPase. The method can be carried out in liquid on isolated components, on a matrix (e.g., filter paper, nitrocellulose, agarose), in cells, on tissue sections, and the like. In accordance with the method, a GEF can bind to the GTPase, which binding will modulate some GTPase activity. For example, as discussed above and below, a KIAA0861 binds to Rho, causing guanine nucleotide dissociation. The effect can be directly on the binding site between the GEF and GTPase, or it can be allosteric, or it can be on only one component (e.g., on the GEF only) Assays for guanine nucleotide dissociation can be readily adapted to identify agents which regulate the activity of a GTPase. The method further relates to obtaining or producing agents which have been identified according to the above-described method. The present invention also relates to products identified in accordance with such methods. Various GEFs and GTPases can be employed, including KIAA0861, mSOS, SO, C3G, lsc, Dbl, Dbl-related proteins, polypeptides comprising one or more DH domains, CDC24, Tiam, Ost, Lbc, Vav, Ect2, Bcr, Abr, Rho (A, B, and C), Rac, Ras, CDC42, chimeras thereof, biologically-active fragments thereof, muteins thereof, and the like.

In general, an interaction can be determined by labeling the test molecule and/or the KIAA0861 molecule, where the label is covalently or non-covalently attached to the test molecule or KIAA0861 molecule. The label is sometimes a radioactive molecule such as ¹²⁵I, ¹³¹I, ³⁵S or ³H, which can be detected by direct counting of radioemission or by scintillation counting. Also, enzymatic labels such as horseradish peroxidase, alkaline phosphatase, or luciferase may be utilized where the enzymatic label can be detected by determining conversion of an appropriate substrate to product. Also, presence or absence of an interaction can be determined without labeling. For example, a microphysiometer (e.g., Cytosensor) is an analytical instrument that measures the rate at which a cell acidifies its environment using a light-addressable potentiometric sensor (LAPS). Changes in this acidification rate can be used as an indication of an interaction between a test molecule and KIAA0861 (McConnell, H. M. et al., Science 257: 1906-1912 (1992)).

In cell-based systems, cells typically include a KIAA0861 nucleic acid or polypeptide or variants thereof and are often of mammalian origin, although the cell can be of any origin. Whole cells, cell homogenates, and cell fractions (e.g., cell membrane fractions) can be subjected to analysis. Where interactions between a test molecule with a KIAA0861 polypeptide or variant thereof are monitored, soluble and/or membrane bound forms of the polypeptide or variant may be utilized. Where membrane-bound forms of the polypeptide are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl-N,N-dimethyl-3-ammonio-1-propane sulfonate.

An interaction between two molecules also can be detected by monitoring fluorescence energy transfer (FET) (see, for example, Lakowicz et al., U.S. Pat. No. 5,631,169; Stavrianopoulos et al. U.S. Pat. No. 4,868,103). A fluorophore label on a first, “donor” molecule is selected such that its emitted fluorescent energy will be absorbed by a fluorescent label on a second, “acceptor” molecule, which in turn is able to fluoresce due to the absorbed energy. Alternately, the “donor” polypeptide molecule may simply utilize the natural fluorescent energy of tryptophan residues. Labels are chosen that emit different wavelengths of light, such that the “acceptor” molecule label may be differentiated from that of the “donor”. Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. An FET binding event can be conveniently measured through standard fluorometric detection means well known in the art (e.g., using a fluorimeter).

In another embodiment, determining the presence or absence of an interaction between a test molecule and a KIAA0861 molecule can be effected by using real-time Biomolecular Interaction Analysis (BIA) (see, e.g., Sjolander & Urbaniczk, Anal. Chem. 63: 2338-2345 (1991) and Szabo et al., Curr. Opin. Struct. Biol. 5: 699-705 (1995)). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In another embodiment, the KIAA0861 molecule or test molecules are anchored to a solid phase. The KIAA0861 molecule/test molecule complexes anchored to the solid phase can be detected at the end of the reaction. The target KIAA0861 molecule is often anchored to a solid surface, and the test molecule, which is not anchored, can be labeled, either directly or indirectly, with detectable labels discussed herein.

It may be desirable to immobilize a KIAA0861 molecule, an anti-KIAA0861 antibody, or test molecules to facilitate separation of complexed from uncomplexed forms of KIAA0861 molecules and test molecules, as well as to accommodate automation of the assay. Binding of a test molecule to a KIAA0861 molecule can be accomplished in any vessel suitable for containing the reactants. Examples of such vessels include microtiter plates, test tubes, and micro-centrifuge tubes. In one embodiment, a fusion polypeptide can be provided which adds a domain that allows a KIAA0861 molecule to be bound to a matrix. For example, glutathione-S-transferase/KIAA0861 fusion polypeptides or glutathione-S-transferase/target fusion polypeptides can be adsorbed onto glutathione sepharose beads (Sigma Chemical, St. Louis, Mo.) or glutathione derivitized microtiter plates, which are then combined with the test compound or the test compound and either the non-adsorbed target polypeptide or KIAA0861 polypeptide, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of KIAA0861 binding or activity determined using standard techniques.

Other techniques for immobilizing a KIAA0861 molecule on matrices include using biotin and streptavidin. For example, biotinylated KIAA0861 polypeptide or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with KIAA0861 polypeptide or test molecules but which do not interfere with binding of the KIAA0861 polypeptide to its test molecule. Such antibodies can be derivitized to the wells of the plate, and unbound target or KIAA0861 polypeptide trapped in the wells by antibody conjugation. Methods for detecting such complexes, in addition to those described above for the GST-immobilized complexes, include immunodetection of complexes using antibodies reactive with the KIAA0861 polypeptide or target molecule, as well as enzyme-linked assays which rely on detecting an enzymatic activity associated with the KIAA0861 polypeptide or test molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., Trends Biochem Sci August; 18(8): 284-7 (1993)); chromatography (gel filtration chromatography, ion-exchange chromatography); electrophoresis (see, e.g., Ausubel et al, eds. Current Protocols in Molecular Biology, J. Wiley: New York (1999)); and immunoprecipitation (see, for example, Ausubel, F. et al., eds. Current Protocols in Molecular Biology, J. Wiley: New York (1999)). Such resins and chromatographic techniques are known to one skilled in the art (see, e.g., Heegaard, J. Mol. Recognit. Winter; 11(1-6): 141-8 (1998); Hage & Tweed, J. Chromatogr. B Biomed. Sci. Appl. October 10; 699 (1-2): 499-525 (1997)). Further, fluorescence energy transfer may also be conveniently utilized, as described herein, to detect binding without further purification of the complex from solution.

In another embodiment, modulators of KIAA0861 expression are identified. For example, a cell or cell free mixture is contacted with a candidate compound and the expression of KIAA0861 mRNA or polypeptide evaluated relative to the level of expression of KIAA0861 mRNA or polypeptide in the absence of the candidate compound. When expression of KIAA0861 mRNA or polypeptide is greater in the presence of the candidate compound than in its absence, the candidate compound is identified as a stimulator of KIAA0861 mRNA or polypeptide expression. Alternatively, when expression of KIAA0861 mRNA or polypeptide is less (statistically significantly less) in the presence of the candidate compound than in its absence, the candidate compound is identified as an inhibitor of KIAA0861 mRNA or polypeptide expression. The level of KIAA0861 mRNA or polypeptide expression can be determined by methods described herein for detecting KIAA0861 mRNA or polypeptide.

In another embodiment, binding partners that interact with a KIAA0861 molecule are detected. The KIAA0861 molecules can interact with one or more cellular or extracellular macromolecules, such as polypeptides, in vivo, and these molecules that interact with KIAA0861 molecules are referred to herein as “binding partners.” Molecules that disrupt such interactions can be useful in regulating the activity of the target gene product. Such molecules can include, but are not limited to molecules such as antibodies, peptides, and small molecules. Target genes/products for use in this embodiment often are the KIAA0861 genes herein identified. In an alternative embodiment, provided is a method for determining the ability of the test compound to modulate the activity of a KIAA0861 polypeptide through modulation of the activity of a downstream effector of a KIAA0861 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), e.g., a substrate, a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases where it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

Also, binding partners of KIAA0861 molecules can be identified in a two-hybrid assay or three-hybrid assay (see, e.g., U.S. Pat. No. 5,283,317; Zervos et al., Cell 72:223-232 (1993); Madura et al, J. Biol. Chem. 268: 12046-12054 (1993); Bartel et al., Biotechniques 14: 920-924 (1993); Iwabuchi et al., Oncogene 8: 1693-1696 (1993); and Brent WO94/10300), to identify other polypeptides, which bind to or interact with KIAA0861 (“KIAA0861-binding polypeptides” or “KIAA0861-bp”) and are involved in KIAA0861 activity. Such KIAA0861-bps can be activators or inhibitors of signals by the KIAA0861 polypeptides or KIAA0861 targets as, for example, downstream elements of a KIAA0861-mediated signaling pathway.

A two-hybrid system is based on the modular nature of most transcription factors, which consist of separable DNA-binding and activation domains. Briefly, the assay utilizes two different DNA constructs. In one construct, the gene that codes for a KIAA0861 polypeptide is fused to a gene encoding the DNA binding domain of a known transcription factor (e.g., GAL-4). In the other construct, a DNA sequence, from a library of DNA sequences, that encodes an unidentified polypeptide (“prey” or “sample”) is fused to a gene that codes for the activation domain of the known transcription factor. (Alternatively the: KIAA0861 polypeptide can be the fused to the activator domain.) If the “bait” and the “prey” polypeptides are able to interact, in vivo, forming a KIAA0861-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the polypeptide which interacts with the KIAA0861 polypeptide.

Candidate therapeutics for treating breast cancer are identified from a group of test molecules that interact with a KIAA0861 nucleic acid or polypeptide. Test molecules are normally ranked according to the degree with which they interact or modulate (e.g., agonize or antagonize) DNA replication and/or processing, RNA transcription and/or processing, polypeptide production and/or processing, and/or function of KIAA0861 molecules, for example, and then top ranking modulators are selected. In a preferred embodiment, the candidate therapeutic (i.e., test molecule) acts as a KIAA0861 antagonist. Also, pharmacogenomic information described herein can determine the rank of a modulator. Candidate therapeutics typically are formulated for administration to a subject.

Therapeutic Treatments

Formulations or pharmaceutical compositions typically include in combination with a pharmaceutically acceptable carrier, a compound, an antisense nucleic acid, a ribozyme, an antibody, a binding partner that interacts with a KIAA0861 polypeptide, a KIAA0861 nucleic acid, or a fragment thereof. The formulated molecule may be one that is identified by a screening method described above. Also, formulations may comprise a KIAA0861 polypeptide or fragment thereof, where the KIAA0861 polypeptide is able to bind to a Rho GTPase but unable to catalyze GDP-GTP exchange reactions of Rho proteins. As used herein, the term “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols such as mannitol, sorbitol, sodium chloride sometimes are included in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation often utilized are vacuum drying and freeze-drying which yields a powder of the active ingredient plus any additional desired ingredient from a previously sterile-filtered solution thereof.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Molecules can also be prepared in the form of suppositories (e.g., with conventional suppository bases such as cocoa butter and other glycerides) or retention enemas for rectal delivery.

In one embodiment, active molecules are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Molecules which exhibit high therapeutic indices often are utilized. While molecules that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such molecules often lies within a range of circulating concentrations that include the ED₅₀ with little or no toxicity. The dosage may vary within this range depending upon the dosage form employed and the route of administration utilized. For any molecules used in the methods described herein, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, sometimes about 0.01 to 25 mg/kg body weight, often about 0.1 to 20 mg/kg body weight, and more often about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, sometimes between 2 to 8 weeks, often between about 3 to 7 weeks, and more often for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment, or sometimes can include a series of treatments.

With regard to polypeptide formulations, featured herein is a method for treating breast cancer in a subject, which comprises contacting one or more cells in the subject with a polypeptide that interacts with a KIAA0861 polypeptide and inhibits its guanine nucleotide exchange factor activity.

For antibodies, a dosage of 0.1 mg/kg of body weight (generally 10 mg/kg to 20 mg/kg) is often utilized. If the antibody is to act in the brain, a dosage of 50 mg/kg to 100 mg/kg is often appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al., J. Acquired Immune Deficiency Syndromes and Human Retrovirology 14:193 (1997).

Antibody conjugates can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a polypeptide such as tumor necrosis factor, .alpha.-interferon, .beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Alternatively, an antibody can be conjugated to a second antibody to form an antibody heteroconjugate as described by Segal in U.S. Pat. No. 4,676,980.

For compounds, exemplary doses include milligram or microgram amounts of the compound per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid described herein, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

KIAA0861 nucleic acid molecules can be inserted into vectors and used in gene therapy methods for treating breast cancer. Featured herein is a method for treating breast cancer in a subject, which comprises contacting one or more cells in the subject with a first KIAA0861 nucleic acid, where genomic DNA in the subject comprises a second KIAA0861 nucleic acid comprising one or more polymorphic variations associated with breast cancer, and where the first KIAA0861 nucleic acid comprises fewer polymorphic variations associated with breast cancer. The first and second KIAA0861 nucleic acids typically comprise a nucleotide sequence selected from the group consisting of the nucleotide sequence of SEQ ID NO: 1-3; a nucleotide sequence which encodes a polypeptide consisting of an amino acid sequence of SEQ ID NO: 4 or 5; a nucleotide sequence that is 90% or more identical to the nucleotide sequence of SEQ ID NO: 1-3, and a nucleotide sequence which encodes a polypeptide that is 90% identical to an amino acid sequence of SEQ ID NO: 4 or 5. The second KIAA0861 nucleic acid also may be a fragment of the foregoing comprising one or more polymorphic variations. The subject often is a human.

Gene therapy vectors can be delivered to a subject by, for example, intravenous injection, local administration (see U.S. Pat. No. 5,328,470) or by stereotactic injection (see e.g., Chen et al., (1994) Proc. Natl. Acad. Sci. USA 91:3054-3057). Pharmaceutical preparations of gene therapy vectors can include a gene therapy vector in an acceptable diluent, or can comprise a slow release matrix in which the gene delivery vehicle is imbedded. Alternatively, where the complete gene delivery vector can be produced intact from recombinant cells (e.g., retroviral vectors) the pharmaceutical preparation can include one or more cells which produce the gene delivery system. Examples of gene delivery vectors are described herein.

Pharmaceutical compositions can be included in a container, pack, or dispenser together with instructions for administration.

Pharmaceutical compositions of active ingredients can be administered by any of the paths described herein for therapeutic and prophylactic methods for treating breast cancer. With regard to both prophylactic and therapeutic methods of treatment, such treatments may be specifically tailored or modified, based on knowledge obtained from pharmacogenomic analyses described herein. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

Administration of a prophylactic agent can occur prior to the manifestation of symptoms characteristic of the KIAA0861 aberrance, such that a disease or disorder is prevented or, alternatively, delayed in its progression. Depending on the type of KIAA0861 aberrance, for example, a KIAA0861 molecule, KIAA0861 agonist, or KIAA0861 antagonist agent can be used for treating the subject. The appropriate agent can be determined based on screening assays described herein.

As discussed, successful treatment of KIAA0861 disorders can be brought about by techniques that serve to inhibit the expression or activity of target gene products. For example, compounds (e.g., an agent identified using an assays described above) that exhibit negative modulatory activity can be used to prevent and/or treat breast cancer. Such molecules can include, but are not limited to peptides, phosphopeptides, small organic or inorganic molecules, or antibodies (including, for example, polyclonal, monoclonal, humanized, anti-idiotypic, chimeric or single chain antibodies, and FAb, F(ab′)2 and FAb expression library fragments, scFV molecules, and epitope-binding fragments thereof).

Further, antisense and ribozyme molecules that inhibit expression of the target gene can also be used to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances where the target gene encodes an extracellular polypeptide, normal target gene polypeptide often is co-administered into the cell or tissue to maintain the requisite level of cellular or tissue target gene activity.

Another method by which nucleic acid molecules may be utilized in treating or preventing a disease characterized by KIAA0861 expression is through the use of aptamer molecules specific for KIAA0861 polypeptide. Aptamers are nucleic acid molecules having a tertiary structure which permits them to specifically bind to polypeptide ligands (see, e.g., Osborne, et al, Curr. Opin. Chem. Biol. 1(1): 5-9 (1997); and Patel, D. J., Curr. Opin. Chem. Biol. June; 1(1): 32-46 (1997)). Since nucleic acid molecules may in many cases be more conveniently introduced into target cells than therapeutic polypeptide molecules may be, aptamers offer a method by which KIAA0861 polypeptide activity may be specifically decreased without the introduction of drugs or other molecules which may have pluripotent effects.

Antibodies can be generated that are both specific for target gene product and that reduce target gene product activity. Such antibodies may, therefore, by administered in instances whereby negative modulatory techniques are appropriate for the treatment of KIAA0861 disorders. For a description of antibodies, see the Antibody section above.

In circumstances where injection of an animal or a human subject with a KIAA0861 polypeptide or epitope for stimulating antibody production is harmful to the subject, it is possible to generate an immune response against KIAA0861 through the use of anti-idiotypic antibodies (see, for example, Herlyn, D., Ann. Med.; 31(1): 66-78 (1999); and Bhattacharya-Chatterjee & Foon, Cancer Treat. Res.; 94: 51-68 (1998)). If an anti-idiotypic antibody is introduced into a mammal or human subject, it should stimulate the production of anti-anti-idiotypic antibodies, which should be specific to the KIAA0861 polypeptide. Vaccines directed to a disease characterized by KIAA0861 expression may also be generated in this fashion.

In instances where the target antigen is intracellular and whole antibodies are used, internalizing antibodies may be utilized. Lipofectin or liposomes can be used to deliver the antibody or a fragment of the Fab region that binds to the target antigen into cells. Where fragments of the antibody are used, the smallest inhibitory fragment that binds to the target antigen often is utilized. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al., Proc. Natl. Acad. Sci. USA 90: 7889-7893 (1993)).

KIAA0861 molecules and compounds that inhibit target gene expression, synthesis and/or activity can be administered to a patient at therapeutically effective doses to prevent, treat or ameliorate KIAA0861 disorders. A therapeutically effective dose refers to that amount of the compound sufficient to result in amelioration of symptoms of the disorders.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices often are utilized. While compounds that exhibit toxic side effects can be used, care should be taken to design a delivery system that targets such compounds to the site of affected tissue in order to minimize potential damage to uninfected cells and, thereby, reduce side effects.

Data obtained from cell culture assays and animal studies can be used in formulating a range of dosage for use in humans. The dosage of such compounds often lies within a range of circulating concentrations that include the ED₅₀ with little or no toxicity. The dosage can vary within this range depending upon the dosage form employed and the route of administration utilized. For any compound used in a method described herein, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of effective dose determination for an individual is the ability to directly assay levels of “free” and “bound” compound in the serum of the test subject. Such assays may utilize antibody mimics and/or “biosensors” that have been created through molecular imprinting techniques. The compound which is able to modulate KIAA0861 activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell et al., Current Opinion in Biotechnology 7: 89-94 (1996) and in Shea, Trends in Polymer Science 2: 166-173 (1994). Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, et al, Nature 361: 645-647 (1993). Through the use of isotope-labeling, the “free” concentration of compound which modulates the expression or activity of KIAA0861 can be readily monitored and used in calculations of IC₅₀. Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. A rudimentary example of such a “biosensor” is discussed in Kriz et al., Analytical Chemistry 67: 2142-2144 (1995).

Provided herein are methods of modulating KIAA0861 expression or activity for therapeutic purposes. Accordingly, in an exemplary embodiment, the modulatory method involves contacting a cell with a KIAA0861 or agent that modulates one or more of the activities of KIAA0861 polypeptide activity associated with the cell. An agent that modulates KIAA0861 polypeptide activity can be an agent as described herein, such as a nucleic acid or a polypeptide, a naturally-occurring target molecule of a KIAA0861 polypeptide (e.g., a KIAA0861 substrate or receptor), a KIAA0861 antibody, a KIAA0861 agonist or antagonist, a peptidomimetic of a KIAA0861 agonist or antagonist, or other small molecule.

In one embodiment, the agent stimulates one or more KIAA0861 activities. Examples of such stimulatory agents include active KIAA0861 polypeptide and a nucleic acid molecule encoding KIAA0861. In another embodiment, the agent inhibits one or more KIAA0861 activities. Examples of such inhibitory agents include antisense KIAA0861 nucleic acid molecules, anti-KIAA0861 antibodies, and KIAA0861 inhibitors, and competitive inhibitors that target Rho family GTP-binding proteins that are regulated by KIAA0861. These modulatory methods can be performed in vitro (e.g., by culturing the cell with the agent) or, alternatively, in vivo (e.g., by administering the agent to a subject). As such, provided are methods of treating an individual afflicted with a disease or disorder characterized by aberrant or unwanted expression or activity of a KIAA0861 polypeptide or nucleic acid molecule. In one embodiment, the method involves administering an agent (e.g., an agent identified by a screening assay described herein), or combination of agents that modulates (e.g., upregulates or downregulates) KIAA0861 expression or activity. In a preferred embodiment, the method involves administering an agent (e.g., an agent identified by a screening assay described herein), or combination of agents that inhibits KIAA0861 expression or activity (e.g., a KIAA0861 activity may include catalyzing the exchange of Rho-bound GDP for GTP). In another embodiment, the method involves administering a KIAA0861 polypeptide or nucleic acid molecule as therapy to compensate for reduced, aberrant, or unwanted KIAA0861 expression or activity.

Stimulation of KIAA0861 activity is desirable in situations in which KIAA0861 is abnormally downregulated and/or in which increased KIAA0861 activity is likely to have a beneficial effect. For example, stimulation of KIAA0861 activity is desirable in situations in which a KIAA0861 is downregulated and/or in which increased KIAA0861 activity is likely to have a beneficial effect. Likewise, inhibition of KIAA0861 activity is desirable in situations in which KIAA0861 is abnormally upregulated and/or in which decreased KIAA0861 activity is likely to have a beneficial effect.

Methods of Treatment

In another aspect, provided are methods for identifying a predisposition to cancer in an individual as described herein and, if a genetic predisposition is identified, treating that individual to delay or reduce or prevent the development of cancer. Such a procedure can be used to treat breast cancer. Optionally, treating an individual for cancer may include inhibiting cellular proliferation, inhibiting metastasis, inhibiting invasion, or preventing tumor formation or growth as defined herein. Suitable treatments to prevent or reduce or delay breast cancer focus on inhibiting additional cellular proliferation, inhibiting metastasis, inhibiting invasion, and preventing further tumor formation or growth. Treatment usually includes surgery followed by radiation therapy. Surgery may be a lumpectomy or a mastectomy (e.g., total, simple or radical). Even if the doctor removes all of the cancer that can be seen at the time of surgery, the patient may be given radiation therapy, chemotherapy, or hormone therapy after surgery to try to kill any cancer cells that may be left. Radiation therapy is the use of x-rays or other types of radiation to kill cancer cells and shrink tumors. Radiation therapy may use external radiation (using a machine outside the body) or internal radiation. Chemotherapy is the use of drugs to kill cancer cells. Chemotherapy may be taken by mouth, or it may be put into the body by inserting a needle into a vein or muscle. Hormone therapy often focuses on estrogen and progesterone, which are hormones that affect the way some cancers grow. If tests show that the cancer cells have estrogen and progesterone receptors (molecules found in some cancer cells to which estrogen and progesterone will attach), hormone therapy is used to block the way these hormones help the cancer grow. Hormone therapy with tamoxifen is often given to patients with early stages of breast cancer and those with metastatic breast cancer. Other types of treatment being tested in clinical trials include sentinel lymph node biopsy followed by surgery and high-dose chemotherapy with bone marrow transplantation and peripheral blood stem cell transplantation. Any preventative/therapeutic treatment known in the art may be prescribed and/or administered, including, for example, surgery, chemotherapy and/or radiation treatment, and any of the treatments may be used in combination with one another to treat or prevent breast cancer (e.g., surgery followed by radiation therapy).

Rho-proteins function as binary switches cycling between a biologically inactive GDP-bound state and a biologically active GTP-bound state. Conversion to the GTP-bound form is mediated by the actions of Rho-GEFs, which stimulate the dissociation of bound GDP, thus providing an opportunity for GTP to bind. Since most Rho proteins exhibit biological activity only when in the GTP-bound state, RhoGEFs, such as KIAA0861, are thought to be Rho activators. Regulation of RhoGEFs are believed to be the result of several regulatory modes involving intra or inter-molecular interactions (Zheng, Y. Trends Biochem. Sci. 26(12): 724-732 (2001)). A first possible RhoGEF regulatory mode is through the intramolecular interaction between DH and PH domains. A second possible RhoGEF regulatory mode is through the intramolecular interaction of a regulatory domain with the DH or PH domain such that the regulatory domain imposes a constraint on the normal DH and/or PH domain functions. A third possible mode involves oligomerization through an intermolecular interaction between Dh domains, thus allowing for the recruitment of multiple Rho substrates into one signaling complex. A final possible mode involves the recruitment of inhibitory cellular factors that suppress GEF activity and help maintain the basal, inactive state. Regulatory molecules known to affect RhoGEF activity include kinases, lipid products from kinase reactions such as phosphoinositol phosphates, and cytoskelelton proteins such as ankyrin (Zheng, Y. Trends Biochem. Sci. 26(12): 724-732 (2001)). Deregulation of any of these regulatory mechanisms due to a specific mutation in KIAA0861 can lead to a Rho protein that is always biologically active, thus leading to oncogenesis.

Thus, featured herein are methods of regulating a biological pathway in which a GTPase is involved, particularly pathological conditions, e.g., cell proliferation (e.g., cancer), growth control, morphogenesis, stress fiber formation, and integrin-mediated interactions, such as embryonic development, tumor cell growth and metastasis, programmed cell death, hemostatis, leukocyte homing and activation, bone resorption, clot retraction, and the response of cells to mechanical stress. See, e.g., Clarke and Brugge, Science 268:233-239, 1995; Bussey, Science, 272:225-226, 1996. Thus, all aspects of modulating an activity of a Rho polypeptide is included herein, which often comprises administering an effective amount of a compound which modulates the activity of a Rho polypeptide, or an effective amount of a nucleic acid which codes for a KIAA0861 polypeptide or a biologically-active fragment thereof. The activity of Rho which is modulated can include: GTP binding, GDP binding, GTPase activity, integrin binding, coupling or binding or Rho to receptor or effector-like molecules (such as integrins, growth factor receptors, tyrosine kinases, PI-3K, PIP-5K, and the like). See, e.g., Clarke and Brugge, Science 268:233-239, 1995. The activity can be modulated by increasing, reducing, antagonizing, promoting, and the like, of Rho. The modulation of Rho can be measured by assayed routinely for GTP hydrolysis, PI(4,5)biphosphate, binding to KIAA0861 or a similar assay such as the one described in Example 11. An effective amount is any amount which, when administered, modulates the Rho activity. The activity can be modulated in a cell, a tissue, a whole organism, in situ, in vitro (test tube, a solid support, and the like), in vivo, or in any desired environment.

Also provided are methods of preventing or treating breast cancer comprising providing an individual in need of such treatment with a GEF inhibitor that reduces or blocks the dysregulated guanine nucleotide exchange function of the GEF in the subject. In some embodiments, it is preferable to specifically reduce or block the dysregulated guanine nucleotide exchange function of KIAA0861 by administering a KIAA0861 inhibitor to the subject in need thereof (e.g., an inhibitor that inhibits the activity of KIAA0861 more than the activity of another GEF). GEF inhibitors and KIAA0861 specific inhibitors sometimes bind to a GEF or KIAA0861 polypeptide or interact with another peptide and reduce the guanine nucleotide exchange function of the GEF or KIAA0861. Also included are methods of reducing or blocking the guanine nucleotide exchange function of KIAA0861 by introducing point mutations into the catalytic domain of KIAA0861 to inhibit its GDP-GTP exchange activity. In the embodiments described above, treating or preventing breast cancer are specifically directed to reducing or inhibiting breast cancer cell metastasis. Thus, featured are methods for reducing or inhibiting breast cancer cell metastasis by inhibiting a GEF or specifically inhibiting KIAA0861. Data shown herein demonstrates that inhibition of KIAA0861 can inhibit cancer metastasis (e.g., see siRNA results herein).

The examples set forth below are intended to illustrate but not limit the invention.

### EXAMPLES

In the following studies a group of subjects were selected according to specific parameters relating to breast cancer. Nucleic acid samples obtained from individuals in the study group were subjected to genetic analysis, which identified associations between breast cancer and a polymorphism in the KIAA0861 gene on chromosome three. Methods are described for producing KIAA0861 polypeptide and KIAA0861 polypeptide variants in vitro or in vivo, KIAA0861 nucleic acids or polypeptides and variants thereof are utilized for screening test molecules for those that interact with KIAA0861 molecules. Test molecules identified as interactors with KIAA0861 molecules and KIAA0861 variants are further screened in vivo to determine whether they treat breast cancer.

### Example 1

### Samples and Pooling Strategies

### Sample Selection

Blood samples were collected from individuals diagnosed with breast cancer, which were referred to as case samples. Also, blood samples were collected from individuals not diagnosed with breast cancer as gender and age-matched controls. All of the samples were of German/German descent. A database was created that listed all phenotypic trait information gathered from individuals for each case and control sample. Genomic DNA was extracted from each of the blood samples for genetic analyses.

DNA Extraction from Blood Samples

Six to ten milliliters of whole blood was transferred to a 50 ml tube containing 27 ml of red cell lysis solution (RCL). The tube was inverted until the contents were mixed. Each tube was incubated for 10 minutes at room temperature and inverted once during the incubation. The tubes were then centrifuged for 20 minutes at 3000×g and the supernatant was carefully poured off. 100-200 μl of residual liquid was left in the tube and was pipetted repeatedly to resuspend the pellet in the residual supernatant. White cell lysis solution (WCL) was added to the tube and pipetted repeatedly until completely mixed. While no incubation was normally required, the solution was incubated at 37° C. or room temperature if cell clumps were visible after mixing until the solution was homogeneous. 2 ml of protein precipitation was added to the cell lysate. The mixtures were vortexed vigorously at high speed for 20 sec to mix the protein precipitation solution uniformly with the cell lysate, and then centrifuged for 10 minutes at 3000×g. The supernatant containing the DNA was then poured into a clean 15 ml tube, which contained 7 ml of 100% isopropanol. The samples were mixed by inverting the tubes gently until white threads of DNA were visible. Samples were centrifuged for 3 minutes at 2000×g and the DNA was visible as a small white pellet. The supernatant was decanted and 5 ml of 70% ethanol was added to each tube. Each tube was inverted several times to wash the DNA pellet, and then centrifuged for 1 minute at 2000×g. The ethanol was decanted and each tube was drained on clean absorbent paper. The DNA was dried in the tube by inversion for 10 minutes, and then 1000 μl of 1×TE was added. The size of each sample was estimated, and less TE buffer was added during the following DNA hydration step if the sample was smaller. The DNA was allowed to rehydrate overnight at room temperature, and DNA samples were stored at 2-8° C.

DNA was quantified by placing samples on a hematology mixer for at least 1 hour. DNA was serially diluted (typically 1:80, 1:160, 1:320, and 1:640 dilutions) so that it would be within the measurable range of standards. 125 μl of diluted DNA was transferred to a clear U-bottom microtiter plate, and 125 μl of 1×TE buffer was transferred into each well using a multichannel pipette. The DNA and 1×TE were mixed by repeated pipetting at least 15 times, and then the plates were sealed. 50 μl of diluted DNA was added to wells A5-H12 of a black flat bottom microtiter plate. Standards were inverted six times to mix them, and then 50 μl of 1×TE buffer was pipetted into well A1, 1000 ng/ml of standard was pipetted into well A2, 500 ng/ml of standard was pipetted into well A3, and 250 ng/ml of standard was pipetted into well A4. PicoGreen (Molecular Probes, Eugene, Oreg.) was thawed and freshly diluted 1:200 according to the number of plates that were being measured. PicoGreen was vortexed and then 50 μl was pipetted into all wells of the black plate with the diluted DNA. DNA and PicoGreen were mixed by pipetting repeatedly at least 10 times with the multichannel pipette. The plate was placed into a Fluoroskan Ascent Machine (microplate fluorometer produced by Labsystems) and the samples were allowed to incubate for 3 minutes before the machine was run using filter pairs 485 nm excitation and 538 nm emission wavelengths. Samples having measured DNA concentrations of greater than 450 ng/μl were re-measured for conformation. Samples having measured DNA concentrations of 20 ng/μl or less were re-measured for confirmation.

Pooling Strategies

Samples were placed into one of two groups based on disease status. The two groups were female case groups and female control groups. A select set of samples from each group were utilized to generate pools, and one pool was created for each group. Each individual sample in a pool was represented by an equal amount of genomic DNA. For example, where 25 ng of genomic DNA was utilized in each PCR reaction and there were 200 individuals in each pool, each individual would provide 125 pg of genomic DNA. Inclusion or exclusion of samples for a pool was based upon the following criteria: the sample was derived from an individual characterized as Caucasian; the sample was derived from an individual of German paternal and maternal descent; the database included relevant phenotype information for the individual; case samples were derived from individuals diagnosed with breast cancer; control samples were derived from individuals free of cancer and no family history of breast cancer; and sufficient genomic DNA was extracted from each blood sample for all allelotyping and genotyping reactions performed during the study. Phenotype information included pre- or post-menopausal, familial predisposition, country or origin of mother and father, diagnosis with breast cancer (date of primary diagnosis, age of individual as of primary diagnosis, grade or stage of development, occurrence of metastases, e.g., lymph node metastases, organ metastases), condition of body tissue (skin tissue, breast tissue, ovary tissue, peritoneum tissue and myometrium), method of treatment (surgery, chemotherapy, hormone therapy, radiation therapy). Samples that met these criteria were added to appropriate pools based on gender and disease status.

The selection process yielded the pools set forth in Table 1, which were used in the studies that follow:

TABLE 1

| | Female CASE | Female CONTROL |
|---|---|---|
| Pool size | 272 | 276 |
| (Number) | | |
| Pool Criteria | case | control |
| (ex: case/control) | | |
| Mean Age | 59.6 | 55.4 |
| (ex: years) | | |

### Example 2

### Association of Polymorphic Variants with Breast Cancer

A whole-genome screen was performed to identify particular SNPs associated with occurrence of breast cancer. As described in Example 1, two sets of samples were utilized, which included samples from female individuals having breast cancer (breast cancer cases) and samples from female individuals not having cancer (female controls). The initial screen of each pool was performed in an allelotyping study, in which certain samples in each group were pooled. By pooling DNA from each group, an allele frequency for each SNP in each group was calculated. These allele frequencies were then compared to one another. Particular SNPs were considered as being associated with breast cancer when allele frequency differences calculated between case and control pools were statistically significant. SNP disease association results obtained from the allelotyping study were then validated by genotyping each associated SNP across all samples from each pool. The results of the genotyping were then analyzed, allele frequencies for each group were calculated from the individual genotyping results, and a p value was calculated to determine whether the case and control groups had statistically significantly differences in allele frequencies for a particular SNP. When the genotyping results agreed with the original allelotyping results, the SNP disease association was considered validated at the genetic level.

It was discovered that females having a cytosine at position 33106 of SEQ ID NO: 1 were predisposed to breast cancer.

SNP Panel Used for Genetic Analyses

A whole-genome SNP screen began with an initial screen of approximately 25,000 SNPs over each set of disease and control samples using a pooling approach. The pools studied in the screen are described in Example 1. The SNPs analyzed in this study were part of a set of 25,488 SNPs confirmed as being statistically polymorphic as each is characterized as having a minor allele frequency of greater than 10%. The SNPs in the set reside in genes or in close proximity to genes, and many reside in gene exons. Specifically, SNPs in the set are located in exons, introns, and within 5,000 base-pairs upstream of a transcription start site of a gene. In addition, SNPs were selected according to the following criteria: they are located in ESTs; they are located in Locuslink or Ensemble genes; and they are located in Genomatix promoter predictions. SNPs in the set also were selected on the basis of even spacing across the genome, as depicted in Table 2.

A case-control study design using a whole genome association strategy involving approximately 28,000 single nucleotide polymorphisms (SNPs) was employed. Approximately 25,000 SNPs were evenly spaced in gene-based regions of the human genome with a median inter-marker distance of about 40,000 base pairs. Additionally, approximately 3,000 SNPs causing amino acid substitutions in genes described in the literature as candidates for various diseases were used. The case-control study samples were of female German origin (German paternal and maternal descent) 548 individuals were equally distributed in two groups (female controls and female cases). The whole genome association approach was first conducted on 2 DNA pools representing the 2 groups. Significant markers were confirmed by individual genotyping.

TABLE 2

| General Statistics | | Spacing Statistics | |
|---|---|---|---|
| Total # of SNPs | 28,532 | Median | 34,424 bp |
| # of Exonic SNPs | 8,497 (30%) | Minimum* | 1,000 bp |
| # SNPs with refSNP ID | 26,625 (93%) | Maximum* | 3,000,000 bp |
| Gene Coverage | 23,874 | Mean | 122,412 bp |
| Chromosome Coverage | All | Std Deviation | 354, bp |

*Excludes outliers

### Allelotyping and Genotyping Results

The genetic studies summarized above and described in more detail below identified an allelic variant associated with breast cancer, set forth in Table 3.

TABLE 3

| | | | Position | | | | | | |
|---|---|---|---|---|---|---|---|---|---|
| SNP | | Chromosome | in SEQ ID | Contig | Contig | Sequence | | Sequence | Allelic |
| Reference | Chromosome | Position | NO: 1 | Identification | Position | Identification | Locus | Position | Variability |
| 2001449 | 3 | 184049849 | 48563 | NT_005612 | 89424094 | NM_015078 | KIAA0861 | intron | G/C |

Assay for Verifying, Allelotyping, and Genotyping SNPs

A MassARRAY™ system (Sequenom, Inc.) was utilized to perform SNP genotyping in a high-throughput fashion. This genotyping platform was complemented by a homogeneous, single-tube assay method (hME™ or homogeneous MassEXTEND™ (Sequenom, Inc.)) in which two genotyping primers anneal to and amplify a genomic target surrounding a polymorphic site of interest. A third primer (the MassEXTEND™ primer), which is complementary to the amplified target up to but not including the polymorphism, was then enzymatically extended one or a few bases through the polymorphic site and then terminated.

For each polymorphism, SpectroDESIGNER™ software (Sequenom, Inc.) was used to generate a set of PCR primers and a MassEXTEND™ primer was used to genotype the polymorphism. Table 4 shows PCR primers and Table 5 shows extension primers used for analyzing polymorphisms. The initial PCR amplification reaction was performed in a 5 μl total volume containing 1×PCR buffer with 1.5 mM MgCl₂ (Qiagen), 200 μM each of dATP, dGTP, dCTP, dTTP (Gibco-BRL), 2.5 ng of genomic DNA, 0.1 units of HotStar DNA polymerase (Qiagen), and 200 nM each of forward and reverse PCR primers specific for the polymorphic region of interest.

TABLE 4
PCR Primers

| Reference | Forward PCR primer | Reverse PCR primer |
|---|---|---|
| SNP ID | (SEQ ID NO: 7) | (SEQ ID NO: 8) |
| rs2001449 | ATGTCAAGTGCACCCACA | AGGAAGAAACTGACGGAAGG |
| | TG | |

Samples were incubated at 95° C. for 15 minutes, followed by 45 cycles of 95° C. for 20 seconds, 56° C. for 30 seconds, and 72° C. for 1 minute, finishing with a 3 minute final extension at 72° C. Following amplification, shrimp alkaline phosphatase (SAP) (0.3 units in a 2 μl volume) (Amersham Pharmacia) was added to each reaction (total reaction volume was 7 μl) to remove any residual dNTPs that were not consumed in the PCR step. Samples were incubated for 20 minutes at 37° C., followed by 5 minutes at 85° C. to denature the SAP.

Once the SAP reaction was complete, a primer extension reaction was initiated by adding a polymorphism-specific MassEXTEND™ primer cocktail to each sample. Each MassEXTEND™ cocktail included a specific combination of dideoxynucleotides (ddNTPs) and deoxynucleotides (dNTPs) used to distinguish polymorphic alleles from one another. In Table 5, ddNTPs are shown and the fourth nucleotide not shown is the dNTP.

TABLE 5

| | | |
|---|---|---|
| Reference | Extend Probe | Term |
| SNP ID | (SEQ ID NO: 9) | Mix |

| | | |
|---|---|---|
| rs2001449 | CACATGCCTGCTCGCCCCC | ACT |

The MassEXTEND™ reaction was performed in a total volume of 9 μl, with the addition of 1× ThermoSequenase buffer, 0.576 units of ThermoSequenase (Amersham Pharmacia), 600 nM MassEXTEND™ primer, 2 mM of ddATP and/or ddCTP and/or ddGTP and/or ddTTP, and 2 mM of dATP or dCTP or dGTP or dTTP. The deoxy nucleotide (dNTP) used in the assay normally was complementary to the nucleotide at the polymorphic site in the amplicon. Samples were incubated at 94° C. for 2 minutes, followed by 55 cycles of 5 seconds at 94° C., 5 seconds at 52° C., and 5 seconds at 72° C.

Following incubation, samples were desalted by adding 16 μl of water (total reaction volume was 25 μl), 3 mg of SpectroCLEAN™ sample cleaning beads (Sequenom, Inc.) and allowed to incubate for 3 minutes with rotation. Samples were then robotically dispensed using a piezoelectric dispensing device (SpectroJET™ (Sequenom, Inc.)) onto either 96-spot or 384-spot silicon chips containing a matrix that crystallized each sample (SpectroCHIP® (Sequenom, Inc.)). Subsequently, MALDI-TOF mass spectrometry (Biflex and Autoflex MALDI-TOF mass spectrometers (Bruker Daltonics) can be used) and SpectroTYPER RT™ software (Sequenom, Inc.) were used to analyze and interpret the SNP genotype for each sample.

Genetic Analysis

Variations identified in the KIAA0861 gene are represented by SEQ ID NO: 1 at position 33106. Minor allelic frequencies for these polymorphisms was verified as being 10% or greater by determining the allelic frequencies using the extension assay described above in a group of samples isolated from 92 individuals originating from the state of Utah in the United States, Venezuela and France (Coriell cell repositories).

Genotyping results are shown for female pools in Table 6A and 6B. Table 6A shows the original genotyping results and Table 6B shows the genotyped results re-analyzed to remove duplicate individuals from the cases and controls (i.e., individuals who were erroneously included more than once as either cases or controls). Therefore, Table 6B represents a more accurate measure of the allele frequencies for this particular SNP. In the subsequent tables, “AF” refers to allelic frequency; and “F case” and “F control” refer to female case and female control groups, respectively.

TABLE 6A

| Reference | | | | |
|---|---|---|---|---|
| SNP ID | AF F case | AF F control | p-value | Odds Ratio |
| rs2001449 | G = 0.703 | G = 0.780 | 0.0040 | 1.49 |
| | C = 0.297 | C = 0.220 | | |

TABLE 6B

| Reference | | | | Odds |
|---|---|---|---|---|
| SNP ID | AF F case | AF F control | p-value | Ratio |
| rs2001449 | G = 0.693 | G = 0.782 | 0.0012 | 1.59 |
| | C = 0.307 | C = 0.218 | | |

As can be seen in Tables 6A and 6B, a cytosine at position 33106 were more common in the female breast cancer group. Genotyping results were considered significant with a calculated p-value of less than 0.05 for genotype results.

Odds ratio results are shown in Tables 6A and 6B. An odds ratio is an unbiased estimate of relative risk which can be obtained from most case-control studies. Relative risk (RR) is an estimate of the likelihood of disease in the exposed group (susceptibility allele or genotype carriers) compared to the unexposed group (not carriers). It can be calculated by the following equation:

RR=IA/Ia

IA is the incidence of disease in the A carriers and Ia is the incidence of disease in the non-carriers.

RR>1 indicates the A allele increases disease susceptibility.

RR<1 indicates the a allele increases disease susceptibility.

For example, RR=1.5 indicates that carriers of the A allele have 1.5 times the risk of disease than non-carriers, i.e., 50% more likely to get the disease.

Case-control studies do not allow the direct estimation of IA and Ia, therefore relative risk cannot be directly estimated. However, the odds ratio (OR) can be calculated using the following equation:

OR=(nDAnda)/(ndAnDa)=pDA(1−pdA)/pdA(1−pDA), or

OR=((case f)/(1−case f))/((control f)/(1−control f)), where f=susceptibility allele frequency.

An odds ratio can be interpreted in the same way a relative risk is interpreted and can be directly estimated using the data from case-control studies, i.e., case and control allele frequencies. The higher the odds ratio value, the larger the effect that particular allele has on the development of breast cancer. Possessing an allele associated with a relatively high odds ratio translates to having a higher risk of developing or having breast cancer.

### Example 3

### Samples and Pooling Strategies for the Replication Samples

SNP reference number rs2001449 was genotyped again in a collection of replication samples to further validate its association with breast cancer. Like the original study population described in Examples 1 and 2, the replication samples consisted of females diagnosed with breast cancer (cases) and females without cancer (controls). The case and control samples were selected and genotyped as described below.

Samples were placed into one of two groups based on disease status. The two groups were female case groups and female control groups. A select set of samples from each group were utilized to generate pools, and one pool was created for each group. Each individual sample in a pool was represented by an equal amount of genomic DNA. For example, where 25 ng of genomic DNA was utilized in each PCR reaction and there were 200 individuals in each pool, each individual would provide 125 pg of genomic DNA. Inclusion or exclusion of samples for a pool was based upon the following criteria: the sample was derived from a female individual characterized as Caucasian; case samples were derived from individuals diagnosed with breast cancer; control samples were derived from individuals free of cancer and no family history of breast cancer; and sufficient genomic DNA was extracted from each blood sample for all allelotyping and genotyping reactions performed during the study. Samples that met these criteria were added to appropriate pools based on gender and disease status.

The selection process yielded the “Griffith” samples set forth in Table 7A and the “Kiechle” samples set forth in Table 7B, which were used in the studies that follow:

TABLE 7A

| | Female CASE | Female CONTROL |
|---|---|---|
| Pool size | 190 | 190 |
| (Number) | | |
| Pool Criteria | case | control |
| (ex: case/control) | | |
| Mean Age | 64.5 | ** |
| (ex: years) | | |

** Each case was matched by a control within 5 years of age of the case.

TABLE 7B

| | Female CASE | Female CONTROL |
|---|---|---|
| Pool size | 195 | 153 |
| (Number) | | |
| Pool Criteria | case | control |
| (ex: case/control) | | |

The replication genotyping results are shown in Table 8A for the Griffith samples and in Table 8B for the Kiechle samples. The odds ratio was calculated as described in Example 2.

TABLE 8A

| Reference | AF | AF | | Odds |
|---|---|---|---|---|
| SNP ID | F case | F control | p-value | Ratio |
| 2001449 | G = 0.685 | G = 0.777 | 0.005 | 1.59 |
| | C = 0.315 | C = 0.223 | | |

TABLE 8B

| Reference | AF | AF | | Odds |
|---|---|---|---|---|
| SNP ID | F case | F control | p-value | Ratio |
| 2001449 | G = 0.754 | G = 0.716 | 0.267 | 0.82 |
| | C = 0.246 | C = 0.284 | | |

The absence of a statistically significant association in the replication cohort should not be interpreted as minimizing the value of the original finding. There are many reasons why a biologically derived association identified in a sample from one population would not replicate in a sample from another population. The most important reason is differences in population history. Due to bottlenecks and founder effects, there may be common disease predisposing alleles present in one population that are relatively rare in another, leading to a lack of association in the candidate region. Also, because common diseases such as breast cancer are the result of susceptibilities in many genes and many environmental risk factors, differences in population-specific genetic and environmental backgrounds could mask the effects of a biologically relevant allele. For these and other reasons, statistically strong results in the original, discovery sample that did not replicate in the replication sample may be further evaluated in additional replication cohorts and experimental systems.

### Example 4

### Mode of Inheritance

To further describe the role of the SNP in breast cancer susceptibility the penetrance was estimated in both the discovery samples and the replication samples to allow inference of the mode of inheritance. The penetrance, defined as the probability of disease given each SNP genotype, was estimated from the case and control genotype frequencies, which provide estimates of the probability of each SNP genotype given the disease. Using Bayes theorem and an assumed age-matched population prevalence of breast cancer (all patients and breast cancer survivors) of 0.028, calculated from NCI data, results reported in Table 9 were obtained.

TABLE 9

| Probability of Breast Cancer | Penetrance | Penetrance |
|---|---|---|
| Based on Genotype | in Discovery | in Replication |
| P(BC\|GG) | 0.022 | 0.022 |
| P(BC\|GC) | 0.035 | 0.035 |
| P(BC\|CC) | 0.048 | 0.048 |

These penetrances suggest that breast cancer susceptibility at this SNP is additive. These penetrances further suggest that breast cancer susceptibility at this SNP is inherited as a dominant trait.

### Example 5

### KIAA0861 Proximal SNPs

It has been discovered that a polymorphic variation (rs2001449) in a gene encoding KIAA0861 is associated with the occurrence of breast cancer (see Examples 1 and 2). Subsequently, SNPs proximal to the incident SNP (rs2001449) were identified and allelotyped in breast cancer sample sets and control sample sets as described in Examples 1 and 2. A total of seventy-five allelic variants located within or nearby the KIAA0861 gene were identified and fifty-seven allelic variants were allelotyped. The polymorphic variants are set forth in Table 10. The chromosome position provided in column four of Table 10 is based on Genome “Build 34” of NCBI's GenBank.

TABLE 10

| | Position in | | | | | |
|---|---|---|---|---|---|---|
| | SEQ ID | | Chromosome | Allele | Genome | Deduced |
| dbSNP rs# | NO: 1 | Chromosome | Position | Variants | Letter | Iupac |
| 3811728 | 246 | 3 | 184201246 | t/c | c | Y |
| 3811729 | 393 | 3 | 184201393 | a/g | a | R |
| 602646 | 628 | 3 | 184201628 | c/g | c | S |
| 488277 | 7586 | 3 | 184208586 | t/c | c | Y |
| 1629673 | 9223 | 3 | 184210223 | a/g | g | R |
| 670232 | 9933 | 3 | 184210933 | a/t | a | W |
| 575326 | 10154 | 3 | 184211154 | t/c | c | Y |
| 575386 | 10175 | 3 | 184211175 | c/g | c | S |
| 684846 | 10877 | 3 | 184211877 | t/c | c | Y |
| 471365 | 10907 | 3 | 184211907 | g/c | g | S |
| 496251 | 11289 | 3 | 184212289 | g/a | a | R |
| 831246 | 11793 | 3 | 184212793 | t/c | t | Y |
| 831247 | 11813 | 3 | 184212813 | g/c | g | S |
| KIAA0861-AA | 13507 | 3 | 184214507 | c/g | g | S |
| 512071 | 14249 | 3 | 184215249 | c/t | c | Y |
| 1502761 | 14586 | 3 | 184215586 | a/c | a | M |
| 681516 | 14647 | 3 | 184215647 | c/t | t | Y |
| 683302 | 15004 | 3 | 184216004 | c/t | t | Y |
| 619424 | 16573 | 3 | 184217573 | t/g | g | K |
| 620722 | 16811 | 3 | 184217811 | a/g | a | R |
| 529055 | 18921 | 3 | 184219921 | a/g | a | R |
| 664010 | 19651 | 3 | 184220651 | t/g | g | K |
| 678454 | 20565 | 3 | 184221565 | c/t | c | Y |
| 2653845 | 25239 | 3 | 184226239 | g/a | a | R |
| 472795 | 25721 | 3 | 184226721 | g/a | a | R |
| 507079 | 27133 | 3 | 184228133 | g/a | g | R |
| 534333 | 27778 | 3 | 184228778 | t/c | t | Y |
| 535298 | 27906 | 3 | 184228906 | t/c | t | Y |
| 536213 | 28000 | 3 | 184229000 | g/a | a | R |
| 831245 | 30005 | 3 | 184231005 | a/g | g | R |
| 639690 | 30520 | 3 | 184231520 | t/c | c | Y |
| 684174 | 32195 | 3 | 184233195 | t/c | c | Y |
| 571761 | 32439 | 3 | 184233439 | c/g | c | S |
| 1983421 | 33858 | 3 | 184234858 | t/c | t | Y |
| 4630966 | 41716 | 3 | 184242716 | c/t | t | Y |
| 2314415 | 42450 | 3 | 184243450 | t/g | c | M |
| 6788196 | 43554 | 3 | 184244554 | g/a | g | R |
| 2103062 | 44211 | 3 | 184245211 | a/g | g | R |
| 9827084 | 44775 | 3 | 184245775 | g/c | c | S |
| 9864865 | 44962 | 3 | 184245962 | a/g | a | R |
| 6804951 | 45317 | 3 | 184246317 | c/t | t | Y |
| 6770548 | 45712 | 3 | 184246712 | a/g | a | R |
| 1403452 | 45941 | 3 | 184246941 | t/c | c | Y |
| 7609994 | 46520 | 3 | 184247520 | g/t | t | K |
| 9838250 | 47175 | 3 | 184248175 | c/t | c | Y |
| 9863404 | 48045 | 3 | 184249045 | g/t | t | K |
| 903950 | 48636 | 3 | 184249636 | c/a | t | K |
| 6787284 | 48689 | 3 | 184249689 | g/a | g | R |
| 2017340 | 48704 | 3 | 184249704 | a/g | c | Y |
| 2001449 | 48849 | 3 | 184249849 | g/c | g | S |
| 1317288 | 48850 | 3 | 184249850 | g/a | g | R |
| 7635891 | 49931 | 3 | 184250931 | t/g | g | K |
| 10704581 | 51510 | 3 | 184252510 | —/tt | t | N |
| 11371910 | 51526 | 3 | 184252526 | —/a | c | N |
| 10937118 | 51758 | 3 | 184252758 | a/g | a | R |
| 7642053 | 51975 | 3 | 184252975 | c/g | g | S |
| 3821522 | 53475 | 3 | 184254475 | a/g | c | Y |
| 2029926 | 55524 | 3 | 184256524 | t/c | g | R |
| 1390831 | 56754 | 3 | 184257754 | t/g | a | M |
| 7643890 | 57473 | 3 | 184258473 | a/g | g | R |
| 11925606 | 57497 | 3 | 184258497 | a/c | c | M |
| 9826325 | 57613 | 3 | 184258613 | g/a | a | R |
| 6800429 | 58023 | 3 | 184259023 | g/a | g | R |
| 6803368 | 58821 | 3 | 184259821 | t/c | c | Y |
| 1353566 | 59644 | 3 | 184260644 | c/a | g | K |
| 2272115 | 66217 | 3 | 184267217 | g/a | a | R |
| 2272116 | 66344 | 3 | 184267344 | g/a | g | R |
| 3732603 | 67326 | 3 | 184268326 | g/c | c | S |
| 940055 | 69777 | 3 | 184270777 | a/c | a | M |
| 2314730 | 83594 | 3 | 184284594 | a/g | g | R |
| 2030578 | 84579 | 3 | 184285579 | g/c | g | S |
| 2049280 | 85623 | 3 | 184286623 | c/t | t | Y |
| 3732602 | 126831 | 3 | 184327831 | c/t | a | R |
| 2293203 | 137878 | 3 | 184338878 | a/t | t | W |
| 7639705 | 147455 | 3 | 184348455 | g/t | t | K |

### Assay for Verifying and Allelotyping SNPs

The methods used to verify and allelotype the seventy-five proximal SNPs of Table 10 are the same methods described in Examples 1 and 2 herein. The PCR primers and extend primers used in these assays are provided in Table 11 and Table 12, respectively.

TABLE 11

| | Forward PCR primer | Reverse PCR primer |
|---|---|---|
| dbSNP rs# | (SEQ ID NOS 10-81) | (SEQ ID NOS 82-153) |
| 3811728 | ACGTTGGATGACGTGTCGGTCCCCTTTCAT | ACGTTGGATGACGCGCCACACCTCCCTAC |
| 3811729 | ACGTTGGATGTGGGCGAGGTTCTGCAGCGT | ACGTTGGATGGTTTCGTTTCTCCGGCACAG |
| 602646 | ACGTTGGATGGAGGAGACCCAGGGTATGAG | ACGTTGGATGTCTGGGACCGTTTACCGCA |
| 488277 | ACGTTGGATGCACACATTCTTCTCAAGTGC | ACGTTGGATGGGAGGGACACAATTTAACTC |
| 1629673 | ACGTTGGATGGGCACCATGTGTGGCTAATT | ACGTTGGATGAAGGATCACGTGAAGTCAGG |
| 670232 | ACGTTGGATGGAAGGTGGAGCAGACATTAG | ACGTTGGATGACCTTAGTTATACCAGGCAC |
| 575326 | ACGTTGGATGACAGAGAGGCTTGGTCATAC | ACGTTGGATGGGTGCTTGGTTGTGATTCTC |
| 575386 | ACGTTGGATGATTCCTGCAGGTACTGTGTC | ACGTTGGATGTGAGCCCAAAACTACTGCTG |
| 684846 | ACGTTGGATGACCACCAGATAAAATCCCTC | ACGTTGGATGAAGTTCCTCTGGTGGACAAC |
| 471365 | ACGTTGGATGTGAGTGACATTTGTGTCACC | ACGTTGGATGCGGAGGATCTGAACAACTTC |
| 496251 | ACGTTGGATGGGGAGTCATTCCAATACCAG | ACGTTGGATGGGAGTGAAAGGTCATATTGG |
| 831246 | ACGTTGGATGCACAATCTGTTAGAATGGTGG | ACGTTGGATGCGTCAAGACTGAATGCATAG |
| 831247 | ACGTTGGATGGAAAATATAGTCCTACACAA | ACGTTGGATGCGTCAAGACTGAATGCATAG |
| KIAA0861-AA | ACGTTGGATGGTTCTAATGTCACCCCTTCC | ACGTTGGATGCAATGTGGCAAATTCTCTGG |
| 512071 | ACGTTGGATGCAAATCACCCCTGACAATTC | ACGTTGGATGACCAGCACACTCAGCTTTAG |
| 1502761 | ACGTTGGATGCAGAAATATGAAGGTGGCCC | ACGTTGGATGACCTTGAGCTCTGAGCCCTT |
| 681516 | ACGTTGGATGCTCCTCCTCAGAGGACTAAC | ACGTTGGATGAGCCCAAGGACTCATACAAC |
| 683302 | ACGTTGGATGAAACATGGCGAAACCCGGTC | ACGTTGGATGACCACGCCTGGCTAATTTTG |
| 619424 | ACGTTGGATGACCGGGAGCTCCCAGTCTG | ACGTTGGATGTGGGAATCGGTTGAGAGCCG |
| 620722 | ACGTTGGATGGCAGCAAAGAATTGCCCGGC | ACGTTGGATGTAAGGCGCCTGCAGAGGCGA |
| 529055 | ACGTTGGATGCTGCAGTTATCTGGGTGAGC | ACGTTGGATGCCAGAACGTGGCTTGTTGGG |
| 664010 | ACGTTGGATGTGGTACCTCCAGGTAAAATG | ACGTTGGATGTCCAGGCAGTCATTTTACCC |
| 678454 | ACGTTGGATGTTCTCTGCGGAGGAAAGTGC | ACGTTGGATGTTAAGCCAGTCCCCACAAGG |
| 2653845 | ACGTTGGATGATCACTTGGACTCAGGAAGC | ACGTTGGATGAGTCTTGCTCTGTTTCCAGG |
| 472795 | ACGTTGGATGTCACCTGAGCATCAGACATG | ACGTTGGATGATAGTGGAAGGAGAAACGGG |
| 507079 | ACGTTGGATGAAGCCTCAGATGAGGCATAC | ACGTTGGATGTCTGAAAGGGTTCAGGAAGG |
| 534333 | ACGTTGGATGCGTTGATGCACTGAAGGGAG | ACGTTGGATGAGAGGCTAAATGTTGGCAGG |
| 535298 | ACGTTGGATGCAATTGCTCAGACCTTCACC | ACGTTGGATGAATGCTAGAGACATTGCACC |
| 536213 | ACGTTGGATGTGAGGACCTCATTATTGGTG | ACGTTGGATGCTGAGCAATCGAACTGCTAC |
| 831245 | ACGTTGGATGCTAGAATTACAGGTGCACAC | ACGTTGGATGGCCAAGATGGTGAAACCTTG |
| 639690 | ACGTTGGATGGCATTTTACCACCATGTGGTT | ACGTTGGATGCCTTCATGTTAATTCTGCCC |
| 684174 | ACGTTGGATGCTTTACTGAGTGGGCAAACG | ACGTTGGATGTCTAAGTGGAACTCAGCAGC |
| 571761 | ACGTTGGATGAATATCCTAGGCTAGCAGTG | ACGTTGGATGGTGCATAAATACATGAATAG |
| 1983421 | ACGTTGGATGTCCAGGTGTTATGGAGTCAG | ACGTTGGATGGGCTTCTTGTGCTGCTGTGT |
| 4630966 | ACGTTGGATGTCAACAAAGATGCCAAGACC | ACGTTGGATGGTGGATATCCATTGTCCTAG |
| 2314415 | ACGTTGGATGGGCTGAGTAACAGTCCATTG | ACGTTGGATGCTTACAGTATCCAAAAAGGG |
| 6788196 | ACGTTGGATGTCAAAGGTAGGTTACCCCTG | ACGTTGGATGATCCCCAATTTGCACATCCC |
| 2103062 | ACGTTGGATGTGCAGCCCTCAACCTTTCAG | ACGTTGGATGCCTTATTCAGTTACTATTACG |
| 9827084 | ACGTTGGATGAAACACACACACCCACATAC | ACGTTGGATGGGGAGAAAGAAAACAAAGGC |
| 9864865 | ACGTTGGATGCAATGCCTGCACTTAGACAC | ACGTTGGATGAGTGATGAGAACATGGGCTG |
| 6804951 | ACGTTGGATGGCAATAGGACTCCCTTTACC | ACGTTGGATGAAGATACGAATGGAGCCTGG |
| 6770548 | ACGTTGGATGTTTTTGAGCTTCACTGAGCG | ACGTTGGATGCGTATCTCTAGCTCAAGCAT |
| 1403452 | ACGTTGGATGCAGAAGTTAGGATGCAGATG | ACGTTGGATGCCAGTAGAGATAGAATTTTGG |
| 7609994 | ACGTTGGATGATACCTAGAGTTTGCCCAAC | ACGTTGGATGAGCTGAGATCAATCCCTATG |
| 9838250 | ACGTTGGATGGTGGCAGTCAAAACACAGTC | ACGTTGGATGACAGAGTAAGACTCCGTCTC |
| 9863404 | ACGTTGGATGGCTATTAGAAAGTCAGAGCC | ACGTTGGATGTGTTCCAGAAGGTGTAGAAG |
| 903950 | ACGTTGGATGCTTCAGTTCAGGGAGAGATC | ACGTTGGATGATAGGGCCCCCAGCATAAAA |
| 6787284 | ACGTTGGATGGCTTTCCCCTAAAGCATCTC | ACGTTGGATGGATCTCTCCCTGAACTGAAG |
| 2017340 | ACGTTGGATGTATTCCACTGCCTGCTTTCC | ACGTTGGATGGAAAACAGGAGGAAGTGGTG |
| 2001449 | ACGTTGGATGATGTCAAGTGCACCCACATG | ACGTTGGATGAGGAAGAAACTGACGGAAGG |
| 1317288 | ACGTTGGATGATGTCAAGTGCACCCACATG | ACGTTGGATGAGGAAGAAACTGACGGAAGG |
| 7635891 | ACGTTGGATGTCTCACCTTGCCTTTGGACG | ACGTTGGATGCTGATGTCGCAAGGAACCAC |
| 10704581 | ACGTTGGATGCCTGTTGAATTATGGAGGAG | ACGTTGGATGCTCTTCTTTCCATGGATCTTC |
| 11371910 | ACGTTGGATGCTCTTCTTTCCATGGATCTTC | ACGTTGGATGCAGCTAATTTCTCCTGACAG |
| 10937118 | ACGTTGGATGATGCAAACTGGCTGGGAATG | ACGTTGGATGGAGGAGGCTGTGAGAAAAGA |
| 7642053 | ACGTTGGATGATGCCCTGGATTGACCTAAC | ACGTTGGATGGGGTTAGGGTGTGTATAAGG |
| 3821522 | ACGTTGGATGAACCCGCACTACAAGATTCC | ACGTTGGATGGTCAGTCCCACATTCAGAAC |
| 2029926 | ACGTTGGATGTCCCGAACATAAAGACTCAG | ACGTTGGATGGGTTGTAATTGGAACATTGG |
| 1390831 | ACGTTGGATGGTCTGCCAAAGTTCCCTTAG | ACGTTGGATGAGGAAAGGGAAGAGAAACCG |
| 7643890 | ACGTTGGATGGACTGTGAGTTATAGGATAC | ACGTTGGATGATGGGTCGGAGGATTTATAG |
| 11925606 | ACGTTGGATGCTCGGCTAAGGTACTCAATA | ACGTTGGATGAGACCACCAAGTAAAATTGC |
| 9826325 | ACGTTGGATGTTGGGTTAATGCAGGGTCTG | ACGTTGGATGCTAGTTCACCTGGGTCTATC |
| 6800429 | ACGTTGGATGCCAAAGCCCATGTTTTAAAAA | ACGTTGGATGGTTTTTCTAAAATATGGGCT |
| 6803368 | ACGTTGGATGAAACCAGCTCAGGCCATTAC | ACGTTGGATGATGCAAAATAAGCTCTGCCC |
| 1353566 | ACGTTGGATGGGTGTACTCTGCCATTTGTC | ACGTTGGATGTGGAGGAGGTTCTAGTACCC |
| 2272115 | ACGTTGGATGAGTTGTGAGTGATTTCAGGG | ACGTTGGATGCAGGCCTTCTTGCTCTTATC |
| 2272116 | ACGTTGGATGCTGTGCCTTCTGAGTAGTTC | ACGTTGGATGATCTGTTGCCTTAGGTTCAC |
| 3732603 | ACGTTGGATGCTCTCAATTCCATCAGTCTC | ACGTTGGATGCTTTACGAATTTCACAACAGG |
| 940055 | ACGTTGGATGTATGCTTCCAGTCTCTGACC | ACGTTGGATGATAGGTAATCCAGTTGGGCC |
| 2314730 | ACGTTGGATGCTCAGGTAATCTGCCTTCTC | ACGTTGGATGCAGGGATAATGAGAACAAATC |
| 2030578 | ACGTTGGATGAACAACCTTACTTCATGCCC | ACGTTGGATGTTCTCCACTTTCTGGTCAAC |
| 2049280 | ACGTTGGATGTGGATACTGAGGGTCAACTG | ACGTTGGATGCTTCCCAACATTTTCGGCTC |

TABLE 12

| | Extend Primer | |
|---|---|---|
| dbSNP rs# | (SEQ ID NOS: 154-225) | Term Mix |
| 3811728 | GTCCCCTTTCATCTAAAC | ACT |
| 3811729 | TCTGCAGCGTGCGGCGA | ACT |
| 602646 | CCAGGGTATGAGCGGAGGA | ACT |
| 488277 | AGTGCACACAGAACATTTAACA | ACT |
| 1629673 | TGTGGAGACAAGGTCTCACT | ACT |
| 670232 | TGGGCAAACAAGCCCAT | CGT |
| 575326 | TGGTCATACCCTTCAAG | ACT |
| 575386 | GAAGGGTATGACCAAGC | ACT |
| 684846 | AGTTGTTCAGATCCTCC | ACT |
| 471365 | TCCAAAACCACCAGATAAAATC | ACT |
| 496251 | GTATTGTCCTCCAGTGA | ACG |
| 831246 | AGAATGGTGGTGTATTTTTAC | ACT |
| 831247 | TAGTCCTACACAATCTGTTA | ACT |
| KIAA0861-AA | GGTATCAGGAAGAGTCA | ACT |
| 512071 | CCCTGACAATTCCAAAACTAA | ACG |
| 1502761 | GGAGGAGGCACTATTAAT | ACT |
| 681516 | GGCCACCTTCATATTTC | ACG |
| 683302 | CAGGAGATCCAGACCATCC | ACG |
| 619424 | TGCGGCCCCCGCCGGGTT | ACT |
| 620722 | GAATTGCCCGGCTCCGAAT | ACT |
| 529055 | GAGCAGGCAGCACAAGT | ACT |
| 664010 | ACCTCCAGGTAAAATGATTAGTT | ACT |
| 678454 | CAGGGATGGTAATTGAC | ACG |
| 2653845 | AAGCGGAGGTTGCAGTGAGC | ACG |
| 472795 | GACATGTCCCTCTCGGCCT | ACG |
| 507079 | GGCAATGTTTGCCCTTT | ACG |
| 534333 | GGGAGAAAGTAACAGGGTC | ACT |
| 535298 | CAGGTGGATGGGGACAC | ACT |
| 536213 | TGGTGTTAAGTGGCGTG | ACG |
| 831245 | CACACCACCACGCCCGGCT | ACT |
| 639690 | CTGCTATTCATTTGTGTAGA | ACT |
| 684174 | CTCTGATGTTACCTCCTCC | ACT |
| 571761 | CTAGGCTAGCAGTGGGGTTG | ACT |
| 1983421 | GGCAGGGAAGAGAAGAGC | ACT |
| 4630966 | AGATGCCAAGACCATTCAAAG | ACG |
| 2314415 | TAGTTGATGAAGATTTGGG | ACT |
| 6788196 | AGGTTACCCCTGCTGACTTT | ACG |
| 2103062 | GAGATCATTTCTCCTTCAAC | ACT |
| 9827084 | CCACACCCATATATATTTATGCT | ACT |
| 9864865 | AAAGATACACCGTTGAGAAGG | ACT |
| 6804951 | GACTCCCTTTACCTTCATGG | ACG |
| 6770548 | CTTCACTGAGCGTGGTGCC | ACT |
| 1403452 | CACAGATGCTCATGGGTCC | ACT |
| 7609994 | GTTTGCCCAACATATAAACAATAA | CGT |
| 9838250 | AACACAGTCAAAATTTTGCTTCA | ACG |
| 9863404 | GAGCCAAGTTTACATCAAGTTTA | CGT |
| 903950 | AGATCACATTGCCAACCCCCA | CGT |
| 6787284 | CCCGTCTCCTGCTGGTCA | ACG |
| 2017340 | CCCTAAAGCATCTCACAGCCCC | ACT |
| 2001449 | CACATGCCTGCTCGCCCCC | ACT |
| 1317288 | CACATGCCTGCTCGCCCC | ACG |
| 7635891 | TGCCTTTGGACGTCTAGCC | ACT |
| 10704581 | TATGGAGGAGTAGATATTGGAA | CGT |
| 11371910 | GAAAATTCCAATATCTACTCCTC | CGT |
| 10937118 | CTGGGAATGAAATTAGGGCAG | ACT |
| 7642053 | GTTCCCTTGACTTTCCTCAG | ACT |
| 3821522 | GCATCTTCAGGAATCTTG | ACT |
| 2029926 | CATAAAGACTCAGCATTCAGC | ACT |
| 1390831 | GGTTAGGAAGAAATCTGTG | ACT |
| 7643890 | ATCTAGATAATAAAGACCACCAA | ACT |
| 11925606 | CTATTAATGGTGTTTGTCTATGG | ACT |
| 9826325 | TAATGCAGGGTCTGCTGGAT | ACG |
| 6800429 | ATCTCTAAGATATAACACTCTAC | ACG |
| 6803368 | GTGCCTGCAAAGAAAGGAAC | ACT |
| 1353566 | TTGTCAGTTATGAGACCTTG | CGT |
| 2272115 | ATACCTCAGAATACAGCTTTTTTT | ACG |
| 2272116 | TCTCATTTCTCCTCTCTTTC | ACG |
| 3732603 | CTCATTTCCACCCTTCT | ACT |
| 940055 | GTCTCTGACCACTTGACCCA | ACT |
| 2314730 | TCCTTCTTCTCTGCTTT | ACT |
| 2030578 | TCATGCCCATTGGGTTAG | ACT |
| 2049280 | GGGTCAACTGTACCAAG | ACG |

### Genetic Analysis of Allelotyping Results

Allelotyping results are shown for cases and controls in Table 13. The allele frequency for the A2 allele is noted in the fifth and sixth columns for breast cancer pools and control pools, respectively, where “AF” is allele frequency. SNPs with blank allele frequencies were untyped (“not AT”).

TABLE 13

| | Position in | | | | | | | Breast Cancer |
|---|---|---|---|---|---|---|---|---|
| | SEQ ID | Chromosome | A1/A2 | | | | | Associated |
| dbSNP rs# | NO: 1 | Position | Allele | Case AF | Control AF | p-Value | OR | Allele |
| 3811728 | 246 | 184201246 | T/C | T = 0.002 | T = 0.003 | 0.952 | 1.28 | C |
| | | | | C = 0.998 | C = 0.997 | | | |
| 3811729 | 393 | 184201393 | A/G | A = 0.968 | A = 0.947 | 0.268 | 0.61 | A |
| | | | | G = 0.032 | G = 0.053 | | | |
| 602646 | 628 | 184201628 | C/G | C = | C = 0.344 | | | |
| | | | | G = | G = 0.656 | | | |
| 488277 | 7586 | 184208586 | T/C | T = 0.9 | T = 0.898 | 0.92 | 0.98 | T |
| | | | | C = 0.100 | C = 0.102 | | | |
| 1629673 | 9223 | 184210223 | A/G | A = 0.93 | A = 0.911 | 0.459 | 0.78 | A |
| | | | | G = 0.070 | G = 0.089 | | | |
| 670232 | 9933 | 184210933 | A/T | A = 0.138 | A = 0.137 | 0.951 | 0.99 | A |
| | | | | T = 0.862 | T = 0.863 | | | |
| 575326 | 10154 | 184211154 | T/C | T = 0.876 | T = 0.869 | 0.753 | 0.94 | T |
| | | | | C = 0.124 | C = 0.131 | | | |
| 575386 | 10175 | 184211175 | C/G | C = 0.224 | C = 0.221 | 0.921 | 0.98 | C |
| | | | | G = 0.776 | G = 0.779 | | | |
| 684846 | 10877 | 184211877 | T/C | T = 0.202 | T = | | | |
| | | | | C = 0.798 | C = | | | |
| 471365 | 10907 | 184211907 | G/C | G = 0.258 | G = 0.262 | 0.88 | 1.02 | C |
| | | | | C = 0.742 | C = 0.738 | | | |
| 496251 | 11289 | 184212289 | G/A | G = 0.841 | G = 0.839 | 0.967 | 0.99 | G |
| | | | | A = 0.159 | A = 0.161 | | | |
| 831246 | 11793 | 184212793 | T/C | T = 0.229 | T = 0.203 | 0.373 | 0.86 | T |
| | | | | C = 0.771 | C = 0.797 | | | |
| 831247 | 11813 | 184212813 | G/C | G = 0.17 | G = 0.178 | 0.755 | 1.06 | C |
| | | | | C = 0.830 | C = 0.822 | | | |
| KIAA0861- | 13507 | 184214507 | C/G | C = 0.745 | C = 0.762 | 0.557 | 1.10 | G |
| AA | | | | G = 0.255 | G = 0.238 | | | |
| 512071 | 14249 | 184215249 | C/T | C = 0.391 | C = 0.363 | 0.376 | 0.89 | C |
| | | | | T = 0.609 | T = 0.637 | | | |
| 1502761 | 14586 | 184215586 | A/C | A = 0.417 | A = 0.409 | 0.799 | 0.97 | A |
| | | | | C = 0.583 | C = 0.591 | | | |
| 681516 | 14647 | 184215647 | C/T | C = 0.762 | C = 0.817 | 0.0906 | 1.39 | T |
| | | | | T = 0.238 | T = 0.183 | | | |
| 683302 | 15004 | 184216004 | C/T | C = 0.729 | C = | | | |
| | | | | T = 0.271 | T = | | | |
| 619424 | 16573 | 184217573 | T/G | T = 0.925 | T = 0.929 | 0.812 | 1.06 | G |
| | | | | G = 0.075 | G = 0.071 | | | |
| 620722 | 16811 | 184217811 | A/G | T = | A = 0.181 | | | |
| | | | | G = | G = 0.819 | | | |
| 529055 | 18921 | 184219921 | A/G | A = 0.398 | A = 0.364 | 0.325 | 0.86 | A |
| | | | | G = 0.602 | G = 0.636 | | | |
| 664010 | 19651 | 184220651 | T/G | T = 0.549 | T = 0.607 | 0.145 | 1.27 | G |
| | | | | G = 0.451 | G = 0.393 | | | |
| 678454 | 20565 | 184221565 | C/T | C = 1.000 | C = 0.985 | 0.0998 | 0.00 | C |
| | | | | T = 0.000 | T = 0.015 | | | |
| 2653845 | 25239 | 184226239 | G/A | G = 0.825 | G = 0.827 | 0.94 | 1.01 | A |
| | | | | A = 0.175 | A = 0.173 | | | |
| 472795 | 25721 | 184226721 | G/A | G = 0.921 | G = 0.921 | 0.983 | 0.99 | G |
| | | | | A = 0.079 | A = 0.079 | | | |
| 507079 | 27133 | 184228133 | G/A | G = 0.166 | G = 0.167 | 0.979 | 1.00 | A |
| | | | | A = 0.834 | A = 0.833 | | | |
| 534333 | 27778 | 184228778 | T/C | T = 0.502 | T = 0.491 | 0.73 | 0.96 | T |
| | | | | C = 0.498 | C = 0.509 | | | |
| 535298 | 27906 | 184228906 | T/C | T = 0.275 | T = 0.228 | 0.127 | 0.78 | T |
| | | | | C = 0.725 | C = 0.772 | | | |
| 536213 | 28000 | 184229000 | G/A | G = 0.726 | G = 0.717 | 0.781 | 0.96 | G |
| | | | | A = 0.274 | A = 0.283 | | | |
| 831245 | 30005 | 184231005 | A/G | A = 0.979 | A = 0.981 | 0.843 | 1.12 | G |
| | | | | G = 0.021 | G = 0.019 | | | |
| 639690 | 30520 | 184231520 | T/C | T = 0.882 | T = 0.892 | 0.65 | 1.10 | C |
| | | | | C = 0.118 | C = 0.108 | | | |
| 684174 | 32195 | 184233195 | T/C | T = 0.698 | T = 0.708 | 0.756 | 1.05 | C |
| | | | | C = 0.302 | C = 0.292 | | | |
| 571761 | 32439 | 184233439 | C/G | C = 0.601 | C = 0.576 | 0.499 | 0.90 | C |
| | | | | G = 0.399 | G = 0.424 | | | |
| 1983421 | 33858 | 184234858 | T/C | T = 0.566 | T = 0.58 | 0.669 | 1.06 | C |
| | | | | C = 0.434 | C = 0.420 | | | |
| 4630966 | 41716 | 184242716 | C/T | C = 0.359 | C = 0.271 | 0.00247 | 0.66 | C |
| | | | | T = 0.641 | T = 0.729 | | | |
| 2314415 | 42450 | 184243450 | T/G | T = 0.974 | T = 0.951 | 0.124 | 0.53 | T |
| | | | | G = 0.026 | G = 0.049 | | | |
| 6788196 | 43554 | 184244554 | G/A | G = 1.000 | G = 1.000 | 0.967 | 0.00 | G |
| | | | | A = 0 | A = 0.000 | | | |
| 2103062 | 44211 | 184245211 | A/G | A = 0.674 | A = 0.642 | 0.381 | 0.87 | A |
| | | | | G = 0.326 | G = 0.358 | | | |
| 9827084 | 44775 | 184245775 | G/C | G = 0.966 | G = 0.928 | 0.0403 | 0.46 | G |
| | | | | C = 0.034 | C = 0.072 | | | |
| 9864865 | 44962 | 184245962 | A/G | A = 0.106 | A = 0.185 | 0.000529 | 1.93 | G |
| | | | | G = 0.894 | G = 0.815 | | | |
| 6804951 | 45317 | 184246317 | C/T | C = 0.96 | C = 0.904 | 0.00573 | 0.40 | C |
| | | | | T = 0.040 | T = 0.096 | | | |
| 6770548 | 45712 | 184246712 | A/G | A = 0.062 | A = 0.159 | 1.12E−05 | 2.86 | G |
| | | | | G = 0.938 | G = 0.841 | | | |
| 1403452 | 45941 | 184246941 | T/C | T = 0.97 | T = 0.932 | 0.0144 | 0.43 | T |
| | | | | C = 0.030 | C = 0.068 | | | |
| 7609994 | 46520 | 184247520 | G/T | G = 0.001 | G = 0.002 | 0.918 | 2.34 | T |
| | | | | T = 0.999 | T = 0.998 | | | |
| 9838250 | 47175 | 184248175 | C/T | C = 0.52 | C = 0.524 | 0.909 | 1.01 | T |
| | | | | T = 0.480 | T = 0.476 | | | |
| 9863404 | 48045 | 184249045 | G/T | G = 0.001 | G = 0.002 | 0.887 | 2.58 | T |
| | | | | T = 0.999 | T = 0.998 | | | |
| 903950 | 48636 | 184249636 | C/A | C = 0.417 | C = 0.406 | 0.739 | 0.96 | C |
| | | | | A = 0.583 | A = 0.594 | | | |
| 6787284 | 48689 | 184249689 | G/A | G = 0.475 | G = 0.501 | 0.416 | 1.11 | A |
| | | | | A = 0.525 | A = 0.499 | | | |
| 2017340 | 48704 | 184249704 | A/G | A = 0.965 | A = 0.945 | 0.195 | 0.63 | A |
| | | | | G = 0.035 | G = 0.055 | | | |
| 2001449 | 48849 | 184249849 | G/C | G = 0.738 | G = 0.797 | 0.0285 | 1.39 | C |
| | | | | C = 0.262 | C = 0.203 | | | |
| 1317288 | 48850 | 184249850 | G/A | G = 1.000 | G = 1.000 | 0.967 | 0.51 | G |
| | | | | A = 0.000 | A = 0.000 | | | |
| 7635891 | 49931 | 184250931 | T/G | T = 0.973 | T = 0.947 | 0.121 | 0.49 | T |
| | | | | G = 0.027 | G = 0.053 | | | |
| 10704581 | 51510 | 184252510 | —/TT | — = 0.998 | — = 0.997 | 0.949 | 0.83 | — |
| | | | | TT = 0.002 | TT = 0.003 | | | |
| 11371910 | 51526 | 184252526 | —/A | — = 1.000 | — = 1.000 | 0.977 | 0.00 | — |
| | | | | A = 0 | A = 0.000 | | | |
| 10937118 | 51758 | 184252758 | A/G | A = 0.495 | A = 0.51 | 0.629 | 1.06 | G |
| | | | | G = 0.505 | C = 0.490 | | | |
| 7642053 | 51975 | 184252975 | C/G | C = 0.002 | C = 0.003 | 0.908 | 1.85 | G |
| | | | | G = 0.998 | G = 0.997 | | | |
| 3821522 | 53475 | 184254475 | A/G | A = 0.504 | A = 0.52 | 0.62 | 1.07 | G |
| | | | | G = 0.496 | G = 0.480 | | | |
| 2029926 | 55524 | 184256524 | T/C | T = 0.001 | T = 0.001 | 0.975 | 2.52 | C |
| | | | | C = 0.999 | C = 0.999 | | | |
| 1390831 | 56754 | 184257754 | T/G | T = 0.057 | T = 0.076 | 0.284 | 1.36 | G |
| | | | | G = 0.943 | G = 0.924 | | | |
| 7643890 | 57473 | 184258473 | A/G | A = 0.001 | A = 0.002 | 0.934 | 2.30 | G |
| | | | | G = 0.999 | G = 0.998 | | | |
| 11925606 | 57497 | 184258497 | A/C | A = 0 | A = 0.001 | 0.956 | | C |
| | | | | C = 1.000 | C = 0.999 | | | |
| 9826325 | 57613 | 184258613 | G/A | G = 0.002 | G = 0.003 | 0.887 | 1.85 | A |
| | | | | A = 0.998 | A = 0.997 | | | |
| 6800429 | 58023 | 184259023 | G/A | G = 0.605 | G = 0.59 | 0.662 | 0.94 | G |
| | | | | A = 0.395 | A = 0.410 | | | |
| 6803368 | 58821 | 184259821 | T/C | T = 0.002 | T = 0.001 | 0.885 | 0.20 | T |
| | | | | C = 0.998 | C = 0.999 | | | |
| 1353566 | 59644 | 184260644 | C/A | C = 0.452 | C = 0.469 | 0.604 | 1.07 | A |
| | | | | A = 0.548 | A = 0.531 | | | |
| 2272115 | 66217 | 184267217 | G/A | G = 0.673 | G = 0.634 | 0.224 | 0.84 | G |
| | | | | A = 0.327 | A = 0.366 | | | |
| 2272116 | 66344 | 184267344 | G/A | G = 0.999 | G = 1.000 | 0.876 | 8.14 | A |
| | | | | A = 0.001 | A = 0.000 | | | |
| 3732603 | 67326 | 184268326 | G/C | G = 0.773 | G = 0.792 | 0.495 | 1.11 | C |
| | | | | C = 0.227 | C = 0.208 | | | |
| 940055 | 69777 | 184270777 | A/C | A = 0.778 | A = 0.803 | 0.356 | 1.17 | C |
| | | | | C = 0.222 | C = 0.197 | | | |
| 2314730 | 83594 | 184284594 | A/G | A = 0.352 | A = 0.311 | 0.183 | 0.83 | A |
| | | | | G = 0.648 | G = 0.689 | | | |
| 2030578 | 84579 | 184285579 | G/C | G = 1.000 | G = 1.000 | 0.963 | 0.33 | G |
| | | | | C = 0.000 | C = 0.000 | | | |
| 2049280 | 85623 | 184286623 | C/T | C = 0.001 | C = 0.005 | 0.576 | 30.94 | T |
| | | | | T = 0.999 | T = 0.995 | | | |

FIG. 1 shows the proximal SNPs in and around the KIAA0861 gene for females. As indicated, some of the SNPs were untyped. The position of each SNP on the chromosome is presented on the x-axis. The y-axis gives the negative logarithm (base 10) of the p-value comparing the estimated allele in the case group to that of the control group. The minor allele frequency of the control group for each SNP designated by an X or other symbol on the graphs in FIG. 1 can be determined by consulting Table 13. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 110-8 were truncated at that value.

Finally, the gene or genes present in the loci region of the proximal SNPs as annotated by Locus Link (http address: www.ncbi.nlm.nih.gov/LocusLink/) are provided on the graph. The exons and introns of the genes in the covered region are plotted below each graph at the appropriate chromosomal positions. The gene boundary is indicated by the broken horizontal line. The exon positions are shown as thick, unbroken bars. An arrow is place at the 3′ end of each gene to show the direction of transcription.

### Additional Genotyping

A total of fourteen SNPs, including the incident SNP, were genotyped in the discovery cohort. The discovery cohort is described in Example 1. Four of the SNPs are non-synonomous, coding SNPs. Two of the SNPs (rs2001449 and rs6804951) were found to be significantly associated with breast cancer with a p-value of 0.001 and 0.007, respectively. See Table 16.

The methods used to verify and genotype the five proximal SNPs of Table 16 are the same methods described in Examples 1 and 2 herein. The PCR primers and extend primers used in these assays are provided in Table 14 and Table 15, respectively.

TABLE 14

| | Forward PCR primer | Revee PCR primer |
|---|---|---|
| dbSNP # | (SEQ ID NOS 226-239) | (SEQ ID NOS 240-253) |
| 7639705 | ACGTTGGATGTGTCAGAAAGCAAACCTGGC | ACGTTGGATGTTACAGGCATTGGAGACAGC |
| 2293203 | ACGTTGGATGCTGCATAATGGTGGCTTTGG | ACGTTGGATGTGTGGGTGTTCACTTTGCAG |
| 3732602 | ACGTTGGATGCCCTCTTGTCAGGAAGTTCT | ACGTTGGATGGAGACAGAGTTGAACTCCCG |
| 2001449 | ACGTTGGATGAGGAAGAAACTGACGGAAGG | ACGTTGGATGATGTCAAGTGCACCCACATG |
| 6804951 | ACGTTGGATGAAGATACGAATGGAGCCTGG | ACGTTGGATGGCAATAGGACTCCCTTTACC |
| 3821522 | ACGTTGGATGCGCACTACAAGATTCCAAGC | ACGTTGGATGTCAGTCCCACATTCAGAACC |
| 2293203 | ACGTTGGATGTGTGGGTGTTCACTTTGCAG | ACGTTGGATGCTGCATAATGGTGGCTTTGG |
| 3811729 | ACGTTGGATGTGGGCGAGGTTCTGCAGCGT | ACGTTGGATGGTTTCGTTTCTCCGGCACAG |
| 534333 | ACGTTGGATGGATGCACTGAAGGGAGAAAG | ACGTTGGATGAGAGGCTAAATGTTGGCAGG |
| 575326 | ACGTTGGATGTGAGCCCAAAACTACTGCTG | ACGTTGGATGATTCCTGCAGGTACTGTGTC |
| 2272115 | ACGTTGGATGCAGGCCTTCTTGCTCTTATC | ACGTTGGATGAGTTGTGAGTGATTTCAGGG |
| 940055 | ACGTTGGATGTATGCTTCCAGTCTCTGACC | ACGTTGGATGGATAGGTAATCCAGTTGGGC |
| 2017340 | ACGTTGGATGGATCTCTCCCTGAACTGAAG | ACGTTGGATGGCTTTCCCCTAAAGCATCTC |
| 571761 | ACGTTGGATGAATATCCTAGGCTAGCAGTG | ACGTTGGATGGTGCATAAATACATGAATAG |

TABLE 15

| | Extend Primer | |
|---|---|---|
| dbSNP # | (SEQ ID NOS 254-267) | Term Mix |
| 7639705 | TGATGCACGTGGAGCAG | CGT |
| 2293203 | GCCCCTGGAAAAGGCCC | CGT |
| 3732602 | GGAAGATGATGAGACTAAAT | ACG |
| 2001449 | CACATGCCTGCTCGCCCCC | ACT |
| 6804951 | TCCCTTTACCTTCATGG | ACG |
| 3821522 | GCATCTTCAGGAATCTTG | ACT |
| 2293203 | GCCCCTGGAAAAGGCCC | CGT |
| 3811729 | GGTTCTGCAGCGTGCGGCGA | ACT |
| 534333 | GAAGGGAGAAAGTAACAGGGTC | ACT |
| 575326 | TGGTCATACCCTTCAAG | ACT |
| 2272115 | ATCTTCTACACATTGATTCAG | ACT |
| 940055 | TCTCTGACCACTTGACCCA | ACT |
| 2017340 | TGGTGACCAGCAGGAGA | ACG |
| 571761 | GGCTAGCAGTGGGGTTG | ACT |

Table 16, below, shows the case and control allele frequencies along with the p-values for all of the SNPs genotyped. The disease associated allele of column 4 is in bold and the disease associated amino acid of column 5 is also in bold. The chromosome positions provided correspond to NCBI's Build 34. The amino acid change positions provided in column 5 correspond to KIAA0861 polypeptide sequence of SEQ ID NO: 4. The corresponding amino acid position in the alternative KIAA0861 polypeptide sequence (SEQ ID NO: 5) can be easily calculated by adding 83 amino acids to the positions provided in column 5.

TABLE 16
Genotpying Results

| | | | | | | | | | Breast |
|---|---|---|---|---|---|---|---|---|---|
| | Position in | | | Amino | | | | | Cancer |
| | SEQ ID | Chromosome | Alleles | Acid | | AF F | | Odds | Associated |
| dbSNP rs# | NO: 1 | Position | (A1/A2) | Change | AF F case | control | p-value | Ratio | Allele |
| 3811729 | 393 | 184201393 | A/G | | A = 0.917 | A = 0.948 | 0.0542 | 1.65 | G |
| | | | | | G = 0.083 | G = 0.052 | | | |
| 575326 | 10154 | 184211154 | T/C | | T = 0.897 | T = 0.885 | 0.545 | 0.88 | T |
| | | | | | C = 0.103 | C = 0.115 | | | |
| 534333 | 27778 | 184228778 | T/C | | T = 0.254 | T = 0.249 | 0.85 | 0.97 | T |
| | | | | | C = 0.746 | C = 0.751 | | | |
| 571761 | 32439 | 184233439 | C/G | | C = 0.487 | C = 0.465 | 0.492 | 0.92 | C |
| | | | | | G = 0.513 | G = 0.535 | | | |
| 6804951 | 45317 | 184246317 | C/T | A819T | C = 0.956 | C = 0.915 | 0.007 | 2.02 | C |
| | | | | | T = 0.044 | T = 0.085 | | | |
| 2017340 | 48704 | 184249704 | G/A | | G = 0.027 | G = 0.042 | 0.203 | 1.57 | A |
| | | | | | A = 0.973 | A = 0.958 | | | |
| 2001449 | 48849 | 184249849 | G/C | | G = 0.693 | G = 0.782 | 0.001 | 1.59 | C |
| | | | | | C = 0.307 | C = 0.218 | | | |
| 3821522 | 53475 | 184254475 | A/G | | A = 0.372 | A = 0.391 | 0.539 | 1.08 | G |
| | | | | | G = 0.628 | G = 0.609 | | | |
| 2272115 | 66217 | 184267217 | A/G | | A = 0.407 | A = 0.444 | 0.246 | 1.16 | G |
| | | | | | G = 0.593 | G = 0.556 | | | |
| 940055 | 69777 | 184270777 | A/C | | A = 0.702 | A = 0.753 | 0.0721 | 1.29 | C |
| | | | | | C = 0.298 | C = 0.247 | | | |
| 3732602 | 126831 | 184327831 | C/T | S506F | C = 0.008 | C = 0.012 | 0.597 | 1.41 | T |
| | | | | | T = 0.992 | T = 0.988 | | | |
| 2293203 | 137878 | 184338878 | A/T | L295Q | A = 0.012 | A = 0.015 | 0.690 | 1.24 | T |
| | | | | | T = 0.988 | T = 0.985 | | | |
| 7639705 | 147455 | 184348455 | G/T | I276L | G = 0.195 | G = 0.189 | 0.794 | 1.04 | G |
| | | | | | T = 0.805 | T = 0.811 | | | |

### Example 6

### KIAA0861 Expression Profile

A cumulative mRNA expression profile was determined for KIAA0861 using a panel of 56 cells and tissues that represent a plurality of cells from different human tissue types. Specifically, RT-PCR was performed in cDNA made from 56 cell lines and 11 normal tissue samples using the following primers: forward, CCAGTCGAAATGGACTTGAG (SEQ ID NO: 268); and reverse, CGCCTTCACAGTCTTCAAAG (SEQ ID NO: 269). The cDNA samples represent a variety of tissue types throughout the human body. The PCR reactions were done in a final volume of 10 μl using Hotstar Taq™ from Qiagen, Inc. Half of the PCR reaction was loaded on a 2% agarose gel to resolve the resulting product. From the expression profiling described above, KIAA0861 expression was found to be ubiquitous across several tissues, including small intestine, bladder, prostate and colon, for example.

### Expression Pattern in Breast Cancer Cell Lines vs Normal Breast Tissue

Quantitative RT-PCR hME was used to measure relative levels of KIAA0861 mRNA in 4 breast cancer cell lines and 2 normal breast tissue cDNA. A 56 Mix is a cDNA mixture from 56 different cell lines representing the major human tissues was used as a positive control. The amount of cDNA used for each reaction was normalized based on expression of a house keeping gene, HMBS. KIAA0861 expressed significantly in MCF7 and MDA-MB-231 cell lines (both breast cancer cell lines), but not significantly in normal breast tissue.

### Example 7

### Inhibition of KIAA0861 Gene Expression by Transfection of Specific siRNAs

RNAi-based gene inhibition was selected as a rapid way to inhibit expression of KIAA0861 in cultured cells. siRNA reagents were selectively designed to target KIAA0861. Algorithms useful for designing siRNA molecules specific for KIAA0861 are disclosed at the http address www.dharmacon.com. siRNA molecules up to 21 nucleotides in length were utilized. Table 17 summarizes the features of two duplexes that were used in the assays described herein. A non-homologous siRNA reagent (siGL2 control) was used as a negative control.

TABLE 17

| | | | |
|---|---|---|---|
| siRNA | siRNA Target | Sequence Specificity | SEQ ID NO: |

| | | | |
|---|---|---|---|
| siGEF1 | KIAA0861 | GAGACAAGTGGAGCTCCGT | 270 |
| siGEF2 | KIAA0861 | GTGGAGCTCCGTAAAGGCA | 271 |
| siGEF3 | KIAA0861 | ATCACCGCACTGCCATCGA | 272 |
| siGEF4 | KIAA0861 | GCATGCTATCCACGGAAGA | 273 |
| SiGEF1 STABLE | KIAA-861 | GAGACAAGTGGAGCTCCGT | 274 |
| siGL2 control | Non-homologous | CGTACGCGGAATACTTCGA | 275 |
| | scrambled control | | |

The siRNAs were transfected in cell lines MCF-7 and T-47D using Lipofectamine™ 2000 reagent from Invitrogen, Corp. 2.5 μg or 5.0 μg of siRNA was mixed with 6.25 μl or 12.5 μl lipofectamine, respectively, and the mixture was added to cells grown in 6-well plates. Their inhibitory effects on KIAA0861 gene expression were confirmed by precision expression analysis by MassARRAY (quantitativeRT-PCR hME), which was performed on RNA prepared from the transfected cells. See Chunming D. and Cantor C. PNAS 100(6):3059-3064 (2003). KIAA0861 gene expression was also determined by flow cytometric analysis of cells stained with a polyclonal chicken antibody specific for KIAA0861. A 50% reduction of KIAA0861 protein was seen in siGEF1-treated cells. Cell viability was measured at 1, 2, 4 and 6 days post-transfection. Absorbance values were normalized relative to Day 1. RNA was extracted with Trizole reagent as recommended by the manufacturer (Invitrogen, Corp.) followed by cDNA synthesis using SuperScript™ reverse transcriptase.

Strong inhibition of cell proliferation of MCF-7 breast cancer cells by siGEF1 was obtained. siGEF1 also strongly inhibited proliferation of another breast cancer cell line, T47D. These effects were consistent in all six experiments performed. Each data point is an average of 3 wells of a 96-well plate normalized to values obtained from day 1 post transfection. The specificity of the active siRNAs was confirmed with a control siRNA, siGL2, which is not homologous to any human sequences.

Long term inhibition of gene expression is desirable in certain cases. Therefore, included herein are embodiments directed to siRNA duplexes described herein (see Tables 31-36) that are less susceptible to degradation. An example of a modification that decreases susceptibility to degradation is in siSTABLE RNA described at the http address www.dharmacon.com. A stable version of siGEF1 was used in an invasion assay described above, except that the cells were replated 14 days after transfection. Inhibition of MDA-MB-231 cell invasion by siGEF1-STABLE was still seen 15 days after transfection. In contrast, the inhibitory effect of the standard version of siGEF1 was no longer apparent at this time and is comparable to that of the control siGL2-treated cells.

siRNA—Starvation Growth Assay

An aliquot of MCF-7 and T47D cells was plated on Boyden chambers with 8 μm pore membranes that are coated with growth-factor reduced matrigel (Becton Dickinson). In addition to growth factors, matrigel contains basement membrane components such as collagens, laminin, and proteoglycans, making it a more physiological growth surface for these breast cell lines. One day after transfection, cells were trypsinized and resuspended in media without serum and plated on top of the matrigel-coated membrane, which is suspended over media containing 5% serum. Cells were allowed to grow for 6 days then fixed in 2% glutaraldehyde and stained with 0.2% crystal violet. Evidence showed that under low serum conditions and on a physiological surface, inhibition of KIAA0861 expression by siGEF1 dramatically inhibits growth of two breast cancer cell lines by 95%. This effect is greater on the matrigel surface than on plastic at a high serum concentration where 50-60% inhibition in proliferation was seen.

siRNA—Invasion Assay

In addition to high proliferative rates, some cancer cells also develop the ability to metastasize. The metastatic potential of tumor cells can be assessed in vitro using Boyden chambers. MCF-7 and T47D cells are not metastatic and therefore do not traverse through the matrigel. For this assay, another cell line was used, MDA-MB-231, which is known to be highly metastatic. Cells in 6-well plates were transfected with 2.5 μg of either siGEF1 or siGL2 as described above. Cells were replated 5 days after transfection on matrigel-coated Boyden chambers suspended on media containing 10% serum. Cells were stained with crystal violet 20 hrs later and photographed. Cells that remain on top of the membrane were scrubbed off and the cells that had invaded through the matrigel and grew on the bottom of the membrane were photographed. Significant inhibition of MDA-MB-231 breast cancer cell invasion by siGEF1 was observed. Duplicate chambers were used in a Wst-1 assay to determine total cell number for both treatments.

### Example 8

### In Vitro Production of KIAA40861 Polypeptides

KIAA0861 Cloning

KIAA0861 cDNA was cloned into a pET28a (Novagen) and pcDNA3.1 vectors (Invitrogen) using a directional cloning method. A KIAA0861 cDNA insert was prepared using PCR with forward and reverse primers having 5′ restriction site tags (in frame) and 5-6 additional nucleotides in addition to 3′ gene-specific portions, the latter of which is typically about twenty to about twenty-five base pairs in length. A Sal I restriction site was introduced by the forward primer and a Sma I restriction site was introduced by the reverse primer. The ends of KIAA0861 PCR products were cut with the corresponding restriction enzymes (e.g., Sal I and Sma I) and the products were gel-purified. The pIVEX 2.3-MCS vector was linearized using the same restriction enzymes, and the fragment with the correct sized fragment was isolated by gel-purification. Purified KIAA0861 PCR product was ligated into the linearized pIVEX 2.3-MCS vector and E. coli cells were transformed for plasmid amplification. The newly constructed expression vector was verified by restriction mapping and used for protein production.

KIAA0861 DH/PH, DH and PH sequences were cloned out of a human brain library and subsequently cloned into pET28a (Novagen) for bacterial expression and pcDNA3.1 vectors (Invitrogen) for mammalian expression and encode a polypeptide domain described herein. In both cases, a directional cloning method was used and the sequences were verified (for use in NIH-3T3 primary focus forming assay and soft agar assay). The table below summarizes the different plasmid constructs.

TABLE 18

| | | EXPRESSION | |
|---|---|---|---|
| | CLONED | VECTOR | |
| GENE | REGION | TYPE | CLONING VECTOR |
| KIAA0861 | DH and PH | bacterial | pET28a NcoI/SalI sites |
| | domains | | |
| KIAA0861 | DH only | bacterial | pET28a NcoI/SalI sites |
| KIAA0861 | PH only | bacterial | pET28a NcoI/SalI sites |
| KIAA0861 | DH and PH | mammalian | pCDNA3.1 EcoRI/XbaI |
| | domains | | sites |
| KIAA0861 | DH only | mammalian | pCDNA3.1 EcoRI/XbaI |
| | | | sites |
| KIAA0861 | PH only | mammalian | pCDNA3.1 EcoRI/XbaI |
| | | | sites |
| KIAA0861 | full length | mammalian | pCDNA3.1 EcoRI/XbaI |
| | ORF | | sites |
| DBS | DH and PH | bacterial | pET28a NcoI/SalI sites |
| | domains | | |
| DBS | DH and PH | mammalian | pCDNA3.1 EcoRI/XbaI |
| | domains | | sites |

Any method well-known in the art may be used to clone and express a target gene. For example, KIAA0861 cDNA may be cloned into a pIVEX 2.3-MCS vector (Roche Biochem) using a directional cloning method as described above. A KIAA0861 cDNA insert is prepared using PCR with forward and reverse primers having 5′ restriction site tags (in frame) and 5-6 additional nucleotides in addition to 3′ gene-specific portions, the latter of which is typically about twenty to about twenty-five base pairs in length. A Sal I restriction site is introduced by the forward primer and a Sma I restriction site is introduced by the reverse primer. The ends of KIAA0861 PCR products are cut with the corresponding restriction enzymes and the products are gel-purified. The pIVEX 2.3-MCS vector is linearized using the same restriction enzymes, and the fragment with the correct sized fragment is isolated by gel-purification. Purified KIAA0861 PCR product is ligated into the linearized pIVEX 2.3-MCS vector and E. coli cells transformed for plasmid amplification. The newly constructed expression vector is verified by restriction mapping and used for protein production.

E. coli lysate is reconstituted with 0.25 ml of Reconstitution Buffer, the Reaction Mix is reconstituted with 0.8 ml of Reconstitution Buffer; the Feeding Mix is reconstituted with 10.5 ml of Reconstitution Buffer; and the Energy Mix is reconstituted with 0.6 ml of Reconstitution Buffer. 0.5 ml of the Energy Mix was added to the Feeding Mix to obtain the Feeding Solution. 0.75 ml of Reaction Mix, 501 of Energy Mix, and 10 μg of the KIAA0861 template DNA is added to the E. coli lysate.

Using the reaction device (Roche Biochem), 1 ml of the Reaction Solution is loaded into the reaction compartment. The reaction device is turned upside-down and 10 ml of the Feeding Solution is loaded into the feeding compartment. All lids are closed and the reaction device is loaded into the RTS500 instrument. The instrument is run at 30° C. for 24 hours with a stir bar speed of 150 rpm. The pIVEX 2.3 MCS vector includes a nucleotide sequence that encodes six consecutive histidine amino acids on the C-terminal end of the KIAA0861 polypeptide for the purpose of protein purification. KIAA0861 polypeptide is purified by contacting the contents of reaction device with resin modified with Ni²⁺ ions. KIAA0861 polypeptide is eluted from the resin with a solution containing free Ni²⁺ ions.

### Example 9

### Cellular Production of KIAA40861 Polypeptides

KIAA0861 nucleic acids are cloned into DNA plasmids having phage recombination cites and KIAA0861 polypeptides and polypeptide variants are expressed therefrom in a variety of host cells. Alpha-phage genomic DNA contains short sequences known as attP sites, and E. coli genomic DNA contains unique, short sequences known as attB sites. These regions share homology, allowing for integration of phage DNA into E. coli via directional, site-specific recombination using the phage protein Int and the E. coli protein IHF. Integration produces two new att sites, L and R, which flank the inserted prophage DNA. Phage excision from E. coli genomic DNA can also be accomplished using these two proteins with the addition of a second phage protein, Xis. DNA vectors have been produced where the integration/excision process is modified to allow for the directional integration or excision of a target DNA fragment into a backbone vector in a rapid in vitro reaction (Gateway™ Technology (Invitrogen, Inc.)).

A first step is to transfer the KIAA0861 nucleic acid insert into a shuttle vector that contains affL sites surrounding the negative selection gene, ccdB (e.g. pENTER vector, Invitrogen, Inc.). This transfer process is accomplished by digesting the KIAA0861 nucleic acid from a DNA vector used for sequencing, and to ligate it into the multicloning site of the shuttle vector, which will place it between the two attL sites while removing the negative selection gene ccdB. A second method is to amplify the KIAA0861 nucleic acid by the polymerase chain reaction (PCR) with primers containing attB sites. The amplified fragment then is integrated into the shuttle vector using Int and IHF. A third method is to utilize a topoisomerase-mediated process, in which the KIAA0861 nucleic acid is amplified via PCR using gene-specific primers with the 5′ upstream primer containing an additional CACC sequence (e.g., TOPO® expression kit (Invitrogen, Inc.)). In conjunction with Topoisomerase I, the PCR amplified fragment can be cloned into the shuttle vector via the attL sites in the correct orientation.

Once the KIAA0861 nucleic acid is transferred into the shuttle vector, it can be cloned into an expression vector having attR sites. Several vectors containing attR sites for expression of KIAA0861 polypeptide as a native polypeptide, N-fusion polypeptide, and C-fusion polypeptides are commercially available (e.g., pDEST (Invitrogen, Inc.)), and any vector can be converted into an expression vector for receiving a KIAA0861 nucleic acid from the shuttle vector by introducing an insert having an attR site flanked by an antibiotic resistant gene for selection using the standard methods described above. Transfer of the KIAA0861 nucleic acid from the shuttle vector is accomplished by directional recombination using Int, IHF, and Xis (LR clonase). Then the desired sequence can be transferred to an expression vector by carrying out a one hour incubation at room temperature with Int, IHF, and Xis, a ten minute incubation at 37° C. with proteinase K, transforming bacteria and allowing expression for one hour, and then plating on selective media. Generally, 90% cloning efficiency is achieved by this method. Examples of expression vectors are pDEST 14 bacterial expression vector with att7 promoter, pDEST 15 bacterial expression vector with a T7 promoter and a N-terminal GST tag, pDEST 17 bacterial vector with a T7 promoter and a N-terminal polyhistidine affinity tag, and pDEST 12.2 mammalian expression vector with a CMV promoter and neo resistance gene. These expression vectors or others like them are transformed or transfected into cells for expression of the KIAA0861 polypeptide or polypeptide variants. These expression vectors are often transfected, for example, into murine-transformed a adipocyte cell line 3T3-L1, (ATCC), human embryonic kidney cell line 293, and rat cardiomyocyte cell line H9C2.

### Example 10

### Transformation of Normal Cells

Plasmid constructs of KIAA0861 and DBS DHPH domains in pcDNA3.1 vector were transfected into NIH-3T3 cells to determine the potential of these genes to transform normal cells. The oncogenic potential of DBS has already been established (Whitehead, I., Kirk, H., and Kay, R. (1995) Oncogene 10:713-721) and was used here as a positive control. Five μg plasmid was transfected into NIH-3T3 cells grown in 25 mm² flasks using Lipofectamine 2000 (Invitrogen). Approximately 10,000 cells were replated 1 day after transfection into 100 mm² dishes in media containing 10% serum. Cells were allowed to grow and express the plasmids for 4 days then media was changed to contain 2% serum. After 7 days growth in low serum, cells were fixed then stained with crystal violet. The low number of colonies that grew in cells transfected with the vector alone compared to those transfected with either KIAA0861 or DBS DH-PH domains indicate that these genes are transforming. Cells plated at 1000/dish show no growth in the vector alone treatment compared to a substantial number of colonies in the KIAA0861 or DBS treatments (data not shown).

To determine if KIAA0861 is able to induce a metastatic phenotype, a population of NIH-3T3 cells transfected with the above plasmids were selected by growth under 400 μg/ml G418 (geniticin) over a period of 2 months. These cells were then used in an in vitro invasion assay described in Example 8. Evidence showed that KIAA0861 as well as DBS transformed non-metastatic NIH-3T3 cells into cells that are able to invade through a matrigel matrix.

### Example 11

### Guanine Nucleotide Exchange Assays

Fluorescence spectroscopic analysis of N-methylanthraniloyl(mant)-GTP incorporation into bacterially purified Rho GTPases was carried out with a tecan XFlour spectrometer at 20° C. Exchange reaction assay mixtures containing 20 mM Hepes (pH 7.5), 50 mM NaCl, 5 mM MgCl₂, and 2 μM relevant GTPase were prepared in a 200 ul volume in a 96-well plate. The relative fluorescence (λₑₓ=370 nm, λₑₓ=465_{+/-}35 nm) was monitored before and after addition of 200 nM bacterially expressed Histidine tag fusions of KIAA0861, Dbs DH-PH domain proteins, or BSA. KIAA0861 and DBS DH-PH domain proteins are active in exchanging guanine nucleotide from the GST-tag fusions of the GTPases, RhoA and Cdc42. Based on the slope of a straight line fitted through the data points, KIAA0861 was equally active on both RhoA and Cdc42, while Dbs was more active on Cdc42 than on RhoA in this in vitro assay.

An alternative nucleotide exchange assay may be used as well, as described below. Guanine nucleotide exchange assays may be performed in 2 ml reactions. Briefly, nucleotide exchange is monitored as the increase in relative fluorescence of the GTP analog mant-GTP upon binding G protein in a reaction buffer containing 20 mM Tris (pH 7.5), 50 mM NaCl, 10 mM MgCl₂, 1 mM dithiothreitol, 50 μg/ml bovine serum albumin, and 10% glycerol. Prior to the addition of GEF, a 1 μM concentration of the appropriate G protein is incubated with 200 nM mant-GTP at 20° C. in a thermostatted cuvette, and fluorescence is measured using a PerkinElmer Life Sciences LS-50B λₑₓ=360 nm; λₑₘ=440 nm; slits=5/5 nm). After equilibration, 10 nM GEF or buffer (uncatalyzed trace) is added.

Test molecules are screened using one or both of these procedures to determine which of them inhibit the guanine nucleotide exchange function of KIAA0861 or a portion thereof. The top ranked inhibitors identified in these screening procedures then are tested in other processes described herein, to determine their effect on cell transformation by KIAA0861 and cell invasion, for example. Top ranked molecules that inhibit cell transformation and/or cell invasion are identified as candidate therapeutics and are administered to animals and humans to determine their safety and therapeutic efficacy on breast cancer.

Provided hereafter is a KIAA0861 genomic sequence (SEQ ID NO: 1). Polymorphic variants are designated in IUPAC format. The following nucleotide representations are used throughout the specification and figures: “A” or “a” is adenosine, adenine, or adenylic acid; “C” or “c” is cytidine, cytosine, or cytidylic acid; “G” or “g” is guanosine, guanine, or guanylic acid; “T” or “t” is thymidine, thymine, or thymidylic acid; and “I” or “i” is inosine, hypoxanthine, or inosinic acid. SNPs are designated by the following convention: “R” represents A or G, “M” represents A or C; “W” represents A or T; “Y” represents C or T; “S” represents C or G; “K” represents G or T; “V” represents A, C or G; “H” represents A, C, or T; “D” represents A, G, or T; “B” represents C, G, or T; and “N” represents A, G, C, or T.

| >3: 184201001-184348700 | |
|---|---|
| 1 | aaagccaaga ctcccattcc taaaccctag ctcaggtctc catctcttaa atccgagtga |
| 61 | cctctacaaa ctctccctga gaaggtgtcc agaacccttt tggaagcgag ggacagtgtc |
| 121 | actgtctttg gggttgacac ctgctctgag taactcacgg aaaacaagtt ccagctggga |
| 181 | agcccttgga cgcgccacac ctccctaccc gcagcccgtc ctgtggcgcc cgggactcca |
| 241 | gagtgYgttt agatgaaagg ggaccgacac gtcagggcca ccgcgggaag cgctgagggc |
| 301 | cactcaccgg ccagggacgc gaagagcgcg gccgccgcgc tgagctgccg gggcatggtg |
| 361 | ggcgctgggc gaggttctgc agcgtgcggc gaRgtccggg caggccccga atcggtgcca |
| 421 | gagaaaccta cctgtgccgg agaaacgaaa ccacctgctt atgagaagca gccgaaaagc |
| 481 | ccgcccaggg ccgctgggcg gggagggaaa ctccgccggc cccctcctac ccctacggag |
| 541 | cagggagggg cggggactcg gcgcagccgc cggggcccgg gcctctggga ccgtttaccg |
| 601 | cacgcgcgtg gtcccggcag cgccggcstc ctccgctcat accctgggtc tcctcctttc |
| 661 | tttttctttt ctttttgaga cgaagtctcg ctctgtcgcc cagggtggag tgcagtggcg |
| 721 | cgatctcggc tcactacaac ctctgcctcc cgggttcaag cgattcttct gcctcagcct |
| 781 | cccgagtagc tgggattaca ggcatgcacc accacacccg gctaattttt gtatttttag |
| 841 | tagagacggg gtgtcaccat attggccagg ctggtctcga gctcctgacc tcgtgattcg |
| 901 | cccgcctcga cctcccaaag tgctgggatt atagacgtga gccaccgagc ccggccaggg |
| 961 | tctcctcttt tatttctttt ctttttattt cttttgtttt gttttgtttt gttttgtttt |
| 1021 | ttgagacaaa gtctcgctct gtcgccaggc tggagtgcag tggcgggatc tcggctcact |
| 1081 | gccctggttc aagcgattct cctgccgcag cctcccgagt agctggggtt acaggcgccc |
| 1141 | gccaccacgc ccagctaatt tttgtatttt agtagagacg gggtttcacc ctgttggcca |
| 1201 | ggctggtctc gatctcctga ccttgtgatc cgcccgcctc ggcctcccaa agtgttggga |
| 1261 | ttacaggcgt gagccactgc gcccggccca gggtctcctc ttttctaaca gctcgggtac |
| 1321 | ctttctggga acccagagac gcttctcagc cgggagaaag ccagccacta ggcgagcagg |
| 1381 | agcctaaaaa cccctaagca ccctgactcc atgtcttccc agggagtctg cggcagccgc |
| 1441 | gctccacgcc caggcctcgc caggaccgcg gtttgcggga agcaacagga gcacagccca |
| 1501 | gaggcgctag gtctggctgg gagctcgcgc tgccgactcc ccggcgtgcg gcgtcgggga |
| 1561 | acctctagga gccttggatt cttcagctgt aaaacggaca taataatgcc cactcccagt |
| 1621 | gtgttttttt attttctttt ttctttttct ttctttgttt ttgtttgttt gtttttgttt |
| 1681 | ttgtttttga gacagggtct cactctgtcg cccaggctgg agggcaatgg cgtgatctcg |
| 1741 | gctcactgca aacttgggtt caggcgattc tcctgcctca gcctccacag tagctgggat |
| 1801 | tacagatgtg cgccaccacg tccggctaat tttttgtatt tttagtagag accaggtttc |
| 1861 | accgtgttgg ccaagctggt ctcaaactcc tgaccccagg tgatccgccc gcctcggcct |
| 1921 | tccaaagatc tgggattaca agcgtgagcc actgtgcctg gccccaggtg gttttacaga |
| 1981 | ccagaaaatc ctggaacaaa aaacacacaa tatcgttttt tttttttttt tggagtcagg |
| 2041 | gtctcgctct atcacccagg ctggagtgca gtggcgtgat ctcggctcac tgcaacttcg |
| 2101 | acctcctggc ctcaagtgag tctcccacct tagcctcctg agtagctggg accaaaggcg |
| 2161 | cgtgccacca cgcccagcta ttttatttta ttttatgtag agaggaggtc tcgctgtgtt |
| 2221 | gcccaggctg gtctcgagtt cctggcctca aatgatcctc ctgcgttagc caaccattgg |
| 2281 | gattacaggc gtaagccacg gcccacggct caacaacgct gacaggcaac cttttaatgt |
| 2341 | cttatctcct tcctctatta attggattgt ctgtcaaaac aacgatgttt tgacagggct |
| 2401 | tgagtcccag tggggaatac acatttaagc agtatattag gagaccctcc ttatcactag |
| 2461 | attgagggct ttcagcctag cctcaaatta ttttctgaaa aataactttg gctacaacta |
| 2521 | ttttgtctta ctatgttgct ccaaacacta atcaagtaaa cttaaccaaa gcttgcagtg |
| 2581 | tgtttcagaa tggaattttt atggtgaaaa gtgagggtta acttgtgcca gtcaacctag |
| 2641 | tttcagcaac tacctgcttt ctgatctttg agacagttta ttcaaaagac gataattaag |
| 2701 | tgggtataga ctgtgtgcca ggcactcttc ttattccatt taagcgccat agccactcta |
| 2761 | tatggacact gttgttatta tcgctgcccc atttcgcaga tggagaaact aagcacaaag |
| 2821 | aagggagttg cccagagtca cttagataat aaataccgaa acctgaccat aaatcttgtc |
| 2881 | tgccttgaga gtctaggatt ttaagcacat agccgggcgc agtggctcac gcctgtaatc |
| 2941 | ccagcacttt aggaggccga ggcgggcgga tcacgaggtc aggagatgga gaccatcctg |
| 3001 | gctaacacag tgaaacccag ttctattaaa aatataaaaa aattagccag gcgtggtggc |
| 3061 | aggtgcatgt agtcccagct actcgggagg ctgaggcagg agaatggtgt gaacccagga |
| 3121 | ggtggagctt gcagtgagcg gagatcgcgc cactgcactc cagcccgggc gacagaagga |
| 3181 | gactccgtct caaaaaaaaa aaaataaata aaataaagct gctcctctta ccctggaaat |
| 3241 | tccaagggat ttaggagctc tgtttcagga accagggtca aagaccaagt attaaaacaa |
| 3301 | aagattctcc tagaactctg gcatataagg attttaggag ctctgtctta gaaactggga |
| 3361 | cagagaccaa agatatatta ttatatcgca gtatcatagt ttattatttt caaaaaacgt |
| 3421 | tttctggctg gtacagtggt tcatgcctat aatccaagca ctttgggagg ccaaggtggg |
| 3481 | agggtcactt gaggccagaa gttcaagtcc agcctgggca acacagggag accctgccac |
| 3541 | tattaaaaat tttttaaatt agctgggcat ggtggcacat gcctgtagtc ccagctactt |
| 3601 | gggaggctga agcaggagga ttgcttgagc ctgagaggtc aagactgtag tgagctgcga |
| 3661 | tcaagcgact gcactctagc ctgggtgaca aagccagacc ctgtgtctaa aaaaaagaaa |
| 3721 | agaagaggaa aaaaaaaggt ttttatttca actaagttgt tggatttatt agcataaagc |
| 3781 | ttttcataac aatcccttgt cttacaatat ctgtagtata ggtagtgatg tcacttcttt |
| 3841 | tattactaat attaataatt tgtattttct ctcttatttc cctgctaata ttaataattt |
| 3901 | gtattttctc tcttttttcc ctgataagtt tggctggagg ttgatcaatt ctattcatgt |
| 3961 | tttcaagaaa aaaaaaaaaa tttcatttca tcgagttcct tcattgttct tatgttttct |
| 4021 | gttttattca tttctacctg atctttatta ttttctttct tctacttaac ttgtgtttta |
| 4081 | tttgctcctc tttcaatagt tttcaaagat ggaacttgcc tgggatatcc cagcacttta |
| 4141 | ggaggctgag gtgggaggat cacctgaggt caggagttca agaccagcct ggccaacatg |
| 4201 | gtgaaactcc gtctctacta aaaatacaaa aattagctgg gtgtggtggt gggcacctgt |
| 4261 | gatcccagct actcgggagg ctgaggcagg agaatcgctt gaacccggga ggtggaggtt |
| 4321 | acagtgagct gagatcacgc cactgcactc cagcctgggt gacaggagct agactctgtc |
| 4381 | tcaaaaaaaa aaaaagaaaa aaaaaaagat ggaagttgag gccagtggta taaaatcttt |
| 4441 | cttcatctct aataaacagg tttactgtta tgaacgtccc tctaattact actttagttg |
| 4501 | gatcccacaa gtttaatatg ttttcatttt catataatta gaaagacttc ctaatttcct |
| 4561 | tttgctatct cctcgactca tggttattta aaatagcatt atttcatttc caaacatata |
| 4621 | gtttttcaga tatctttctt ttattgattt ttaattccac tgtggttgaa aacataatta |
| 4681 | tggacttaaa tcttacaaat ttattgatat ttgttttttg accaagacta tggttggtta |
| 4741 | cattagtttt caggactgac ataacaaagt gccacagact gggtaattaa accacagaca |
| 4801 | tttgctttct tataattctg gaaaccagac atgtgagatc aaagtgtcag ccgggttggt |
| 4861 | tttttctttt tccttttttt tttttttttt tgagacagag tctctttctg tcacccaggc |
| 4921 | tgggatgcag tggtgtgatc tcggcttact gcaaattctg cctcccaggc tcaagcgatt |
| 4981 | ctcctgcctc agcctcccga gtagctggga ttacaggtgc ctctcactgc acctggctaa |
| 5041 | tttttgtatt tttagtagag acggggtttc accatgttgg ccaggttgat ctcaaactcc |
| 5101 | tgatctcagg taatacaccc gcctcggcct cccaaagtac tgagattaca ggcgtgagcc |
| 5161 | actgcacccg gcccgggtta gttctttcta aggcctctct ccttggctag tagacacctt |
| 5221 | tgtttcacat ggtcatccct ctgtgcatgc ctttgtctgt cctaatctcc tcttcttata |
| 5281 | aggacattag tcaggtagga ttagtgccta ctctttgaac tcgttttacc tcttaaagac |
| 5341 | cctatctccg aatatagtca cattctgaga tacttggggt taagacttgt attagtccat |
| 5401 | tttcacgctg ctcataaaga catacctgag actgggaaga aaaagaggtt taattggaca |
| 5461 | attccacatg gctggggagg cctcagaatc atggtgggag gcgaaaggga ctttttacat |
| 5521 | ggtggcggca agagaaaatg aggaagaagc aaaagcagaa acccctgata gataagccca |
| 5581 | ccagatatca tgagatttat tcactgtcat gagaacagca cgggaaagac cagcccccat |
| 5641 | gaatacatta cctcttcctt ggtccccccc tccccacaat atgtggggat tctgggagat |
| 5701 | acaattaaaa ttgagatttg agtggggaca cagccaaacc atatcattct gtccctggtc |
| 5761 | cttccaaatc tcatgtcctc acatttcaaa accaatcatg cctttccaat agtccctcaa |
| 5821 | agtcttaact catttcagca ttaacctaaa agtccacagt ccaaagtctc atctgagaca |
| 5881 | aggccttccg cctatgagcc tgtacaatca aaagcaagct agttagttcc tagatacaat |
| 5941 | gggggtacag gtattgggta aataaagcca ttccaaatgg gagaaattgg ccaaaacaaa |
| 6001 | ggggttacag ggcccatgca agtctgaaat ccagtgaggg agtcaaattt taaagctcca |
| 6061 | aaatgatctc ctttgactcc aggtcttaca tccaggtcac gctaatgcaa aaggtaggtt |
| 6121 | tccatggtct tgggcagctc cacccctgtg gctttgcagg gtacagcctc cctccaggct |
| 6181 | gctttcatgg gctggtgttg agtgtctgca gcttttccag gcacccagtg caagctgtca |
| 6241 | gtggatctac cattctgggg tttggaggac aaaggccctc ttctcacagc tgcactaggc |
| 6301 | agtgccccga tagggactct gtgtgggggc tctgatccca catttccctt ctgcactgcc |
| 6361 | ctaagaggtt ctccttgagg gccccacagc ttccaccctc tgaaccatag cccaagctat |
| 6421 | gcattggccc ctttcagcca tggctggagc agctgggaca gagggcacca agtcactagg |
| 6481 | ctgcacacaa catggggacc ctgggcctgc cccacaaaac ccctttttcc tcctgggcct |
| 6541 | ccaagcctgt gatgggagag gctgctgtga aggtctctga catggccttg gagacatttc |
| 6601 | cccatggtct tggggattca cattaggctt cttgctactt atgcaaattt ctgcaaccag |
| 6661 | cttgaatttc tccccagaaa atgggttttt cttttctgtc acatagtccg gctgcaaatt |
| 6721 | ttccaaactt ttatgctctg cttcccttat aaaactgaac gcctttaata gcacccaaat |
| 6781 | cacctcttga atgttttgct gcttagaaat tttttccacc agatacccta aataatctct |
| 6841 | caagttcaaa gttccacaag tctctagggc aggggcaaaa tgtggccagt ctctttgcta |
| 6901 | aaacataaca agaggcacct ttgctccagt tcccaaaaag ttcctcatct ccatctgaga |
| 6961 | ccacctcagc ctggatctta ttgtccatat cactatcagc attttgggca aaaccattca |
| 7021 | acaagtctct aggaagttcc aaactttccc acattttcct gtcttcttct gagcccttca |
| 7081 | aactgttcca atctctgcct gttacccagt tccaaagttg ttccacattt tcaggtatct |
| 7141 | tcagcaacgt ttcactctac tggtagcaat ttactgtatt agtccatttt cacactgctg |
| 7201 | ataaagacat atctgagact gggaagaaaa ataggtttaa ttggacttac agttccacat |
| 7261 | ggctggggag gcctcagaat catggtgaga ggtgaaaggc acttcttacg tggtagtgac |
| 7321 | aagagaaaat gaggaagaag caaaagcgga aacccctgat aaatccatca gatctcatga |
| 7381 | gacttattca ctatcacgag aatagcatgg gaaagaccgg cccccatgat tcaattacct |
| 7441 | ccccctgggt ccctcccaca acacatggga attctgggag atacaattca agttgagatt |
| 7501 | tgggtgggga cacagccaaa ccatatcaag acttctacat atgaattttg gagggacaca |
| 7561 | atttaactca taatagtgga ctgtcYtgtt aaatgttctg tgtgcacttg agaagaatgt |
| 7621 | gtgtattctc tcattgttgg attcagtgac ctataaatgt taattaggtt aaactaattg |
| 7681 | atgtagggaa aagaaagaga gatcagactg tcactgtgtc tatgtagaaa gggaagacat |
| 7741 | aagagactcc attttgaaaa agacctgtac ttcaaacaat tgctttgctg agatgttaat |
| 7801 | ttgtagcttt gccccagcca ctttgcccca gccactttga cccaacttgg agctcacaaa |
| 7861 | aacatgtgtt gtataaaatc aaggtttaag ggatctaggg ctgtgcagga cgtgccttgt |
| 7921 | taacaaaatg tttacaagca gtatacttgg tcaaagtcat cgccattctc tagtctcaat |
| 7981 | aaaccagggg cacaatgcac tgcggaaagc tgcagggagc cctgcccttg gaagcggggt |
| 8041 | attgtccaag gtttctcccc atgtgacagt ctgaaatatg gcctcgtagg atgagaaaga |
| 8101 | cctgactgtc ccccagccca acacccataa agggtctgtg ctgaggtgga ttggtaaaag |
| 8161 | aggaaagcct cttacagttg agatagagga aggccactgt ctcctgcctg cccctgggaa |
| 8221 | ctgaatgtct tggtgtaaaa cccgattgta catttgttca actctgaaat aggagaaaag |
| 8281 | ctgccctgtg gtgggaggtg agacatgttt gcagtaatgc tgccttgtta ttctttactc |
| 8341 | cactgagatg tttgggtgga gagaaacata aatctggcct atgtgcacat ccaggcatag |
| 8401 | taccttccct tgaacttaat tatgatacag attcttttgc tcacatgttt tttgttgacc |
| 8461 | ttctccttat tatcaccctg ctgtcctact acattccttt ttgctgaaat aatgaaaata |
| 8521 | ataatcaata aaaactgagg gaactcagag gctggtgccg gtacaggtcc ttggtgtgct |
| 8581 | gagtgccggt cccctggact cactgttgtt tctttatact ttgtctctgt gtcttatttc |
| 8641 | ttttctccgg ctctcatccc acccgactag aaatacccac aggtgtggag gggcaggcca |
| 8701 | ccccttcaat tgatagtatg gttcaagaac aaatggtatc aacttaggat ggtttaactt |
| 8761 | atgatttttc aactttagaa tggtgtgaag tctgtatgca ttcagtagaa ggcatacttt |
| 8821 | gaatttttat cttttcccaa gctactgcta tgggacaaga tactctctca caattctggg |
| 8881 | cagtggcagc aagcctcagc ttccagtcag cacccaatcc caagggtaaa caactgatac |
| 8941 | agccattctg tttttcattt ttagcaaaat actcaataaa ttacatgagg cactcaatgc |
| 9001 | tttattataa gacaagcttt gtattagatg atttgcccaa ctgtaggcta atgtaggtgt |
| 9061 | tctgagcaca tttaaggtag actatactat gccatgatgt ttggaaggtt aggtaaattt |
| 9121 | aatgcatttt cgacttagaa tactttcagc ctcccaaata gctgggacca caggtgtgtg |
| 9181 | gcaccatgtg tggctaattt tttgtggaga caaggtctca ctRtgtcgcc caagttggtc |
| 9241 | tcaaaatcct gacttcacgt gatccttcca ctctggtctc ccaaagtgcg attacaggtg |
| 9301 | tgagtcacca cacctggccg tgttgtcaca cattttaatt ctttataaat tagaaacttc |
| 9361 | acaacacatt gttcttattt aaaatttaaa caattatctt taaatataca taatatataa |
| 9421 | atatgtatat aatatataaa tatatacaat atataaatat ataagatata ttatatatat |
| 9481 | aatatataaa tatataatat ataaatatat aatatgtaaa tatataatat ataaatatat |
| 9541 | gtaaatatgt aatatgtaaa tatgtaatat gtaaatatat aatatgtaaa tatataatat |
| 9601 | ataaatatac ataatatata aatatataat atataaatat ataatatata aatatataat |
| 9661 | atataaatat ataatatata aatatatatt atatataaat atataatata cataaatata |
| 9721 | tataatatat aaatatatat aatatacata aatatataat atataaatat atataatata |
| 9781 | tataaatata tatggggaaa aaagctttta tacttactca tgtgattacc gtttcttgca |
| 9841 | gtctttattc ctttgtttag atccttggga tttttgttgc tatggtgacc ttagttatac |
| 9901 | caggcacttc aaatcttacc ttgtgtttag gaWatgggct tgtttgccca aaggtcttcc |
| 9961 | ctaatgtctg ctccaccttc aactttaggt cttctctgct gtcagtttct ctctccatac |
| 10021 | acttgtagct ctcccaggag tattccatcg ttacttgtta gtcagtgctt attagcgggg |
| 10081 | tggtgggatc tgagaggtga ggtgcttggt tgtgattctc agttctgatt cctgcaggta |
| 10141 | ctgtgtccct gggYcttgaa gggtatgacc aagcStctct gtccctcccc gcagcagtag |
| 10201 | ttttgggctc agcacatatt cctgcccctt ccccagaatc agagggtttt ttgttgttgt |
| 10261 | tgttttgttt ttcaagtttt tgttcctttt tctccatctg tgttggattt accagccccc |
| 10321 | taggagcgtc agtatttgtt acccttcctc caggctttta aggcctttgt aggagagatg |
| 10381 | ggccaatagc atctgagcat ggttttgtgt ctttcttgta gcaactgata ttcctcaccc |
| 10441 | ccaggtctat gccaggaagg atgcttcctt acatgcctgt aatcccagct accggggagg |
| 10501 | ctgaagcaga agaatcactt gaacccagga ggcagaggtt gcagtgagcc aagatcatgc |
| 10561 | cactgcactc cagcctggca acagagcgag acttcattca aaaaaaaaaa aagctcttac |
| 10621 | actcttctgc atattctgca aagtatactc tgggaaggct ggtttagagg atctctaact |
| 10681 | tttctaatat ttcataatgc atggctgatg tttaacatga actcagactc ccatcagatt |
| 10741 | ttacagatct gggtctgctt ttagttctaa tgcttcagcc agggcacttt aatctgaggt |
| 10801 | cacagcattc ccaattgtca gcagtatttt gaaatctaag ttcctctggt ggacaacagg |
| 10861 | caacatcata tagccaYgga ggatctgaac aacttctaag tctgagSgat tttatctggt |
| 10921 | ggttttggat tcttttccat ttatacttaa acaaggtgac acaaatgtca ctcacagaaa |
| 10981 | agccactctt ttttcccctg tttttaagaa gtctaaaaat gcacaactat ttcctgaatg |
| 11041 | agcagtgtgt ccaggtgtag aatagggaat gtcgtgtcca ctgggaacag acggtgtacc |
| 11101 | tggcacaatt ttatgagaat tatcccatga ttcctgtccc accctgcagc ccaccatggg |
| 11161 | ctcagtgggg agtctgagtc tctgagcccc aaagggtagc tttttcccag acgacaccag |
| 11221 | caagcagagc agcagcttgg ggctttgaca aggaggctgg agtgaaaggt catattggac |
| 11281 | catcctggRt cactggagga caatacccca ggaacctcca gcaatgactg gtattggaat |
| 11341 | gactcccctg gtcacccacc tgtggcaaga catttgcaaa cagggtcttc ctatgaggct |
| 11401 | tgtgaggttt ggccaatttg cattctgcta gaattattaa gtatggacat cctcatagat |
| 11461 | ccagaaaagg gccgagatcc aaaggattga gaatgctttg gggggtgttt ccatagtgag |
| 11521 | tggctggcta ttcaacagtc aaatgtttga gaggatacaa gtgatgtgtt tcctgaggca |
| 11581 | agtggtcaga acccaacaga ctcttctgtt cagctaagat gagagaccat ctgaagttct |
| 11641 | tacatgttct cacagagaaa tggattctca tagggaaatg gatatattgt gatagacact |
| 11701 | gtaagcagag aagttgactg agaaacacac acacacacac acacacacac acacacgtca |
| 11761 | agactgaatg catagatgtg tattataaaa agYgtaaaaa tacaccacca ttStaacaga |
| 11821 | ttgtgtagga ctatattttc ttttttttta atttattatt attatacttt aagttttagg |
| 11881 | gtacatgtgc acaatgtgca ggttagttac atatgtatac atgtgccatg ctggtgtgct |
| 11941 | gcacccatta actcgtcatt tagcattagg tatatctcct aatgctatcc ctcccccctt |
| 12001 | cccccacccc acaacagtcc ccagaatggg atgttcccct tcctgtgtcc atgtgttctc |
| 12061 | attgttcaat tcccacctat gagtgagaat atgtggtgtt ttgttttttg tccttgcaat |
| 12121 | agtttactga gaatgatgat ttccaatttc atccatgtcc ctacaaagga catgaactca |
| 12181 | ccatttttta tggctgcata gtattccatg gtgtatatgt gccacatttt cttaatccag |
| 12241 | tctatcattg ttggacattt gggttggttc caagtctttg ctattgtgaa tagtgccaca |
| 12301 | ataaacatac gtgtgcatgt gtctttatag cagcatgatt tatagtcctt tgggtatata |
| 12361 | cccagtaatg ggatggctgg gtcaaatggt atttctagtt ctagatccct gaggaatcgc |
| 12421 | cacactgact tccacaatgg ttgaactagt ttacagtccc accaacagtg taaaagtgtt |
| 12481 | cctatttctc cacatcctct ccagcacctg ttgtttcctg actttttaat gattgccatt |
| 12541 | ctaactggtg tgagatggta tctcactgtg gttttgattt gcatttctct gatggccagt |
| 12601 | gatgatgagc attttttcat gtgttttttg gctgcatcag tgctctacac gttcagagaa |
| 12661 | acttctctag tgacgaacta tagaaatgat ccctgaaagt atagtcttag gactatattt |
| 12721 | tcttttgact tgggaggcat gtttattgct gttaatgctg caaagggctc tacgtgcttt |
| 12781 | aaaaaatccc aatctgttgc attcataagc ctgggttgga tctaaagcag cctcccactt |
| 12841 | ttggaaaggc atccccacga cctttccatg gttgctgaat gcagctggag gcagtcacag |
| 12901 | ctggtgatgt ccggagccca ttccccactg tgctggtctg cagaacttct gcatgccatt |
| 12961 | cccacaagca ggtctctgcc ctgctctcct ccacctccct tgtcagagga agtctgcact |
| 13021 | tcacagcttt ctggtctcaa ccctcctcca tccctacaga tgtgtaagca gcaggaatca |
| 13081 | aaaggtgaag gagagggggc aactcacctc cgatggacac gtgaaaagtg ggagatggat |
| 13141 | aaaatcaaga aggagcttaa gatatccaga aatgtaaact gtgtttggaa aagtaaggtc |
| 13201 | aggagaagca tgggactcct gaggttgctc cctactatct tgcagacttg ctgcaggacc |
| 13261 | aaatgaagca ggatctgtca agcaccaggg ccagctctta agcttagtgc ctttctgaac |
| 13321 | cctgtgaccc agcagcctcc atcaactcgt cctacctgcc atgcacagct cctctgtgcc |
| 13381 | cctgtacctg agctcatgct attccctctg ccaggatgcc cttctccttc tccaccagga |
| 13441 | gaagaacact tgccagtaag acccagttct aatgtcaccc cttcctgacg gtatcaggaa |
| 13501 | gagtcaStga tggtgtttta tgctcccaga gaatttgcca cattgtgttg tgattatttt |
| 13561 | tccacatctg tctcccccac tggaatgaga gcctcactca tcttcatacc tccctggtct |
| 13621 | ctacctggtg ccagaaccat cctcagggca ggggaatgct caggaaatag atattgaata |
| 13681 | aaataagtgt atccatccat ccatccatcc atccttccat ccatccagac atatatacat |
| 13741 | atgtaaacat tctgatggtc aaatggaaca atgtgggctg aagaataatg caggtagaag |
| 13801 | aacctaagat tacagattct tgatttggga ggaactttat tttattgtgc aaacagtcta |
| 13861 | caaatttgaa acatactcta tcaagaaaga cactgttgtt aggaaagggc gtgggagagg |
| 13921 | gtggccccac aaaacagtgt aaagttctaa agaatttgag aggaacactc tgcgggaccc |
| 13981 | tgttccaagg gcatctttct aagagtctgt cccttaggct ctgccccttt ttggccacgt |
| 14041 | tgtcaaaggc cttctttatg aacgaagtga gacacattat ttttgttttc ttgaattcta |
| 14101 | aagtgattgt cttgagttcc ctgggggaga ctcctgggag gtgtggccca ctctattcca |
| 14161 | gaaacaaacc aggagactga agactcatcc caatcccata tcccatgaaa atgtgaaatc |
| 14221 | aaatcacccc tgacaattcc aaaactaaYg gaatcctaac ataactctat gtaactctaa |
| 14281 | agctgagtgt gctggtaatt aagcttccag cccacccttc ccagctctgg ggtgcagggt |
| 14341 | caccatgggc tccttcctgt gtgcacccca ccccaccccc actgtcagca tcctgtctcc |
| 14401 | accctgagag tgacagctgg ttccagtagc gggagttggt tccagcttgc cattttccta |
| 14461 | gcactcctat aaccagcctg ctgatgccca ttcagagaca gcagcacggg ctggccatgt |
| 14521 | cccctctcca gaattctgcg tccagctcct ggaccttgag ctctgagccc ttgggccacg |
| 14581 | tgtacMatta atagtgcctc ctcctcagag gactaacccc cagccctagg gccaccttca |
| 14641 | tatttcYgag ttttgatatt ttcaacctct tttctttgtt gtatgagtcc ttgggctggg |
| 14701 | agcttgcagt caaaatcttc atgatatctc attatcacta ctttttttta aatctctact |
| 14761 | agctggataa caattattta tattaaattc tctcttgaaa taactgatac agtgtctctt |
| 14821 | gattgaaact tgactagtag actaagaatt ctaactctaa ataattctga gggccgggtg |
| 14881 | tggtggctca cacctgtaat cccagcactt tgggaggcca acgcgaatgg atcacctgag |
| 14941 | gtcaggagtt ggagaccagc ctggcaaaca tggcgaaacc cggtcaggag atccagacca |
| 15001 | tccYggctaa catggtgaaa cctcatcttt acttaaaaaa aaaaaaaaat acaaaacaaa |
| 15061 | attagccagg cgtggtggca gatgcctgta gtcccagcta ttcaggaggc tgagacagga |
| 15121 | gaatagcgtg aacccgggag gtggagcttg cagtgagccg agattgcacc actgcactcc |
| 15181 | agcctgggtg acagagagag actccgtctc aacaacaaca acaacaaatt agccgggcat |
| 15241 | ggtggcaggt gcctgtaatc tcaactactt gggaggctga ggtaggagaa ttgcttgaac |
| 15301 | ccgggaggcg gaggttgcag tgagccaaga ttgcggcact tcactccagc ctgggtgaca |
| 15361 | agagcaaaac tccatctcaa aaaaaaaaaa aaagaaaaaa aaatctgaca attaaataaa |
| 15421 | gaacagaaaa aaaatttgaa tggcaaatac aaagctgaaa agaaataact gagaataaat |
| 15481 | aactctgaaa atagctcaaa aactaaatac ctcaaaaact ctttaaaaat tcagaaaata |
| 15541 | taatgttcaa atatgaagta atgctgaaaa tgaaataact aaaaaccaag taacttgaaa |
| 15601 | aaaagaacat aaaaataaac aactcaaata taaaataact gaaaataaat acctgtgaac |
| 15661 | ataagcaact cgaaaaccag ataactaggg gaaacccttc attaaaacat ttcactctga |
| 15721 | aaataaataa cttgacagta gttcatgaac ttccagtgag tgtttaatag tcaaataagt |
| 15781 | tactgtaaaa ataaataact caaaaactcc aataagataa agtgaaataa ctatgaaggt |
| 15841 | aaataactca gataataatt gtaaagataa ttaaaaataa attccggctg ggcgcggtgg |
| 15901 | ctcacgcctg taatcccagc actttgggag gccgaggcag ggggatcacg aggtcaggag |
| 15961 | atcgagacca tcctggctaa cacggggaaa ccccgtctct actaaaaatc caaataaaaa |
| 16021 | attagccggg cgtggtggcg ggcgcctgta gtcccagcta ctcaggaggc tgaggcagga |
| 16081 | gaatggcgtg aacccgggag gcggtttgca gtgagccgag atcacaccac tgcactccag |
| 16141 | cctgggcgac agagcgagac tccgtctcga aaaataaata aataaataaa taaataaatt |
| 16201 | ccaagcaaat taatttgaac atctgtaact cctaaaacaa aacaactaca tataaataac |
| 16261 | tgaaaatgaa caactaaata actttgaaaa taaagtaact ataaaccagt aactgaaact |
| 16321 | ataaaattaa ataaccagaa agctacaaag aaatgaaaat caactaataa aactaaaaat |
| 16381 | aaaatacaac tgaaactaac aaaactcatt taaaaactaa taaatcgata cataaaatac |
| 16441 | ctcagaaagt aacttggaac gaactctctc tacaacgaag caatcttggc actaacacga |
| 16501 | acaccccatg aacgctcgca gggatgctgg gaggcggacc gggagctccc agtctgcggc |
| 16561 | ccccgccggg ttKgagcggc tccggctcct ccaaggctcg ggctaagcgg ctctcaaccg |
| 16621 | attcccaccc cgccctggag aaatgcgggc gtgtctgcag gcatttttga ttgtcacgat |
| 16681 | ttgggggcgg ggtggaggat ggggtggcgc attcctggca cctagtgggt agaggccagg |
| 16741 | ggtgctacta aacatcctac cgtgggagca cagagaagcc cacgcagcaa agaattgccc |
| 16801 | ggctccgaat Rtcgaagtgc gcggtcgaga aggcgtgggc tgcgggctct gctcgcctct |
| 16861 | gcaggcgcct tagagcagct ccgaggtccc ccgtgcggag ctaggcgcgc acccaggaca |
| 16921 | cccctcgggc tcctcggagg aggccctggt tgtccccttt ctgccgccgc cgaggctccg |
| 16981 | gctgctttct gcgtagctgg gcagggcccg ggcccccaca ccgcctctcc cgggaatgcg |
| 17041 | ggcgctctgg agccgaggag cgggggcgtc cgcagggagg tcagctctcc tgggcggagg |
| 17101 | tcctcgggcg cagcgccctc gcctggaaac cagccgtcgc ccccgcagga gccagccggc |
| 17161 | ccgtggacgc cccagcgcgc tcctcctcgg tgctgcgggt cgccctgcaa ttccgagaag |
| 17221 | aaagtcagag acgccgtggc ccaaagaggc gcttagtctt tcctcgctca cactcacgtt |
| 17281 | tcctcctcat cgcgttcttc tttttctccc tggctgcttt ctcccctctc caggaaagca |
| 17341 | gatttggagg aacaggtttc gtgactgtcg tccgactgga aaaggcccgc gagctggaag |
| 17401 | ggaggggacg ggtgcaccct cagagttatt gctggaggct gtggccagac cgggcaaggt |
| 17461 | ggtgactccc gctggcaggc tgaggcccac cccagccctc ccacctgggc cacggggctc |
| 17521 | tcagcgggag ccccagttat gaccggacac cagcgcaccg ccaaggagac agccacgtgg |
| 17581 | ggacatgctg gactaggagg gtcagagcca gtttgagggt ctggtgacct cggctccctg |
| 17641 | gcttaaccag gtccttatgg gtgagaatcc tgaggagggg gagagggatg gaggctaggg |
| 17701 | acaggaggca ggaggagctg atgaatagga aggagggaag aagatgaaag aaacaaaagg |
| 17761 | gaagtaatta cactcagagc actgctctcc ttttccatgt cttctgcgcc ttcacagtct |
| 17821 | tcaaaggtgt ccatggagct aaactccctg ctggagcagc cttagggaga gaaagggaag |
| 17881 | gatggggcct ctgtggggtg ggaggacatc ccctctgccc atggcagggt gtagcaggca |
| 17941 | gtgcctgttg caggcacggt cctccccatc tctaactcct gctctccaag ggcctgcact |
| 18001 | gcgctgggct gtgagggggt ctgtgatctc caggctgctt ttccagcgcc gagatgccgt |
| 18061 | aattcaccga gaaggcgcgt ccacatgctg ttcatggccc tacctccccg ttcctccaag |
| 18121 | aaaacagtca ttgttttttg tgtttgccag tcttctaacc acgcctcctt cccttcctcc |
| 18181 | tcccctgtct ctttctcacc ctcccctcct tgcctccttt tctctcccct aattaatgtc |
| 18241 | catttcccat ctccctggca gcctctgcca agtgtcactg ctccccataa gggaaaatca |
| 18301 | gaggaacaag caagtgcatc catcctgcct ctctctgcag tgaactgatt aattaatcca |
| 18361 | tcagtcttgt ctatggcgca catgttacat ccctggggcg gtgttggaca ctgtggggaa |
| 18421 | cagcagccac tgccaaatac tgaacaactg ccctgtgcct ggtggtatgt taggcatttg |
| 18481 | tcaaagttta agcctcacaa ccctgtaagg gtctcagccc cctttacagt tggggaaaca |
| 18541 | gacagcaatg gtcacttggc caagtcctct tggcctgtgg cagggcagct gtcttctcca |
| 18601 | gcactcctgc tctttaccct cgctctgagt gagatggagt ctctgcccca catggctcac |
| 18661 | aagccagggt agggagcaga gcatctgcag aaaggtccca acacaggaca gccccagacc |
| 18721 | gagggtcagc tgagtagtct acgcggcggg agccgtgcta ggaaagtgtc tgctcaggcg |
| 18781 | agaattcagg gaggtttggg caggacttga ggggcagaca ggactggaga gggagggcat |
| 18841 | tctgggcaga ggcatggcca gagggcggta ggcggcagtg gagggagctg cagttatctg |
| 18901 | ggtgagcagg cagcacaagt Rgcgtctcct gggctgctgc cccaagcccc caacaagcca |
| 18961 | cgttctgggc cccaggccct ccccagagca gatcagtggg ggctgtgtga gtaacatggg |
| 19021 | ggcggggggg cagctgggca gcacctccct ggaggcccct ctgaaatcct gcctgactct |
| 19081 | ggcaggctcc gagggggctg gacaccctcc tctcaggttg aagcaagtcc tggttgagtt |
| 19141 | cctagtccca ggaggtggga ggggcaaggg gtggagggca gaggagaaac tgcctcaggg |
| 19201 | atgtgccccc tgccttcatc ctccagacag gacttgggag catctaagga aacccaagac |
| 19261 | tcctctttag agaagtcatc cagccctggg gtccccttat gccaggagca agcagtgaga |
| 19321 | atggaagaat gattgtcttg ctgaaagttc tgtgatggag ggatagaggg acagagggag |
| 19381 | ccatgccctt gaccatcccc tgcatgaata ggaagggctg tgtctccagg gtccatggcc |
| 19441 | tctgtgcccc ggatgatgcc agggctgcta gggaccatag agccacccac tgggaggctg |
| 19501 | gcggttgggc ctggctcagg agccttcgtc agccataggc agccacagcc tggggtgggc |
| 19561 | agggctggga ggcgacacag gaactgaaaa acctgacaag ctctagcccc tccgcagggt |
| 19621 | aagtggtacc tccaggtaaa atgattagtt Kgttccagcc cctctgcagg gtaagtggca |
| 19681 | cctggggtaa aatgactgcc tggagctggc agctgctttc cctgctctcg cgggccctgc |
| 19741 | agggaagcgg ggaagggaag ggggcacagc gctgggcaca gaggggctct cagaccctgg |
| 19801 | actcaactgt ttcagggtca tctgaaacag tcaactgttt cctctagccc attccctgcc |
| 19861 | tccaggcgag gatttgcctg aacgtggaaa gaggaaggat cctcccagtg ctgtcaaccc |
| 19921 | cagattccac ctccctgtgg gggactgtca gcgcaggccc tgacaacgca gagaaagaca |
| 19981 | caggacccac ctgggccagt gacagcagga gctccgggtg ccacaggtga gggtggggat |
| 20041 | gcctggagca ccacgggggg cctggtttag tctagagcca ggttttccat acaccttaga |
| 20101 | gtgcaacctc agggagatgc aaattttacc ccctaacaca gcatacacgc agaaacacat |
| 20161 | ttatacaatt caaacacaag cggacggaac aatatttacc cttagagtgt gtgaagtcct |
| 20221 | catctgtccc acctcatcct atcatgcttt gttctatcct aggagacaaa gcaggagggg |
| 20281 | gggctgcgga ggtgggggag tctcatccaa gcccttgggt gacacgtctc tcctgagaca |
| 20341 | aactgcagct gctctgggtg tgccctcgcc tgtctccctc caggccccgg gttcctgcca |
| 20401 | gcagagacag taacctattc accaggtatc ccccagggct cctggaagaa actcagaatt |
| 20461 | ctcagaacca gaaaacctta gagagcatcc tgcaggcaaa gcccctggtt ctctgcggag |
| 20521 | gaaagtgcgg ctcacagggt gccccgccag ggatggtaat tgacYaccag gctgtgtgcc |
| 20581 | ttgtggggac tggcttaagg ccctgtggga gctgagtcag ggccaggacc ggggtgtcct |
| 20641 | gactcctaga gatcatgttc ccttcctcac ccaggccttc cagtcccagc cctgggcttt |
| 20701 | tatttattta ttttggagac agagtctggc ttgtcaccca ggttggagtg cagttgtgtg |
| 20761 | atcacggctc actgcagcct tgacttcctg ggttgaactg atcctcccac atcagcctcc |
| 20821 | tgagtagctg ggaccacagg cacatgccac cacacccagc taatttttgt attttttggt |
| 20881 | agagatgggg ttttaccatg ttggccaggc tggtcttgaa ctcctgagct caactgatct |
| 20941 | gcccacctca gcctcccaca gtgctgggat tacagatgtg agccatcatg cccacctcct |
| 21001 | gggctgactt ttgctgtctt acatcatctg catatttaat cccctgctgg attcactggt |
| 21061 | catgggctct gaggccctaa gagtcttagg cactaaggag ctggcagcac tgaggggacc |
| 21121 | ccaaaatctc agactcagga tctggccagt cacaggcatg tgagggaaca actgagaggc |
| 21181 | ccattgcccc atggcaggag aaggtgctct ggagtcagtc agacctgagg gcagtcagac |
| 21241 | ctgattctca ctctgtcact cactagctgt gtgatcttgg atacatcact taacctcttg |
| 21301 | agcatcagct tccttatctc taaaatggag ataataacat cgattttgca gtcttggtat |
| 21361 | gaggattagc aaatcttctg ataaagaaaa atgcctggta catcatagga attcaacaaa |
| 21421 | tagtacctgt tatgattatt gtgtatagca attacaataa tactaaagag agggtctcaa |
| 21481 | aacagctctg ggcactccag gtgtgctatt attacttaca tttcagggag gtttgtctgc |
| 21541 | cattgtctca tcctcataaa cactcaggga aagaaacatt ataaggataa taaatggctt |
| 21601 | taaaaagaaa cagagcaaac acacacacac acacacccct cagaaaaacc atgccaaaca |
| 21661 | cacaggctct tgacaaatat tcaatctgat tatagcaaaa ctgttttgtt ttgttttgtt |
| 21721 | tttttgtttg tttttgagac aggggtctcg ctctgtcgcc caggctggag tgcagtggcg |
| 21781 | tgatcttggc tcacagcaac ctccttctcc cgggttcaag caattctcct gtctcagcct |
| 21841 | ccagagtagc tgggactaca ggcacatgcc actatgcctg gctaattttt gtatttttag |
| 21901 | tagagacagg gtttcaccat attggtcagg ctggtctcga actcttgatc tcaggtgatc |
| 21961 | cacctgcctt ggcctcccaa agtgtgagat tacaggcgtg agccaccatg cccagctgat |
| 22021 | tatagcaaaa ttctaagtga tagttgtatt cttggaaaat gaatggaacg acttttgtcc |
| 22081 | cagccaagat ctagtggtgt gttggagcag atacaccctg agtctctggg gcactctcag |
| 22141 | tctatgtatc agataagcat aaggagtatt ggtggagaag gacaagaatg ggaaaggtgg |
| 22201 | gcgatgagaa ttcctgcaga taaggactgg tgagagtatt ctcttttgaa acccttagtc |
| 22261 | gacaatcttt ctggtgcata tcagataagc tgaatggttt aggaaatcta gtgttcacat |
| 22321 | ttagtgctta gaattctaag cttttttatt ttgcttaaac aaatggaatg aaatttatta |
| 22381 | acaagtgaac ctagtaatga gctgaaatta ttctcaccag catacatatt tttggtaaat |
| 22441 | tatagacttt gaagacaaaa tcatggtgtt tcccttactg tccagtggat ggcacaaaga |
| 22501 | gaccattgta gatcctgctg gttcagcggt agctctcaat ccatagatat taaatgggca |
| 22561 | aattctattt ttattgtctt tcaaactaga tttttctcaa tgcacaactt tttttttctt |
| 22621 | tttctttttt ttttttgaga cggagtctcg ctctattgcc aggctggagt gcagtgacac |
| 22681 | gatctcggct cactgcaacc tccgcctccc gggttcaagt gattctcctg cctcagcctc |
| 22741 | ctgagtagct ggggactaca ggcgcatgcc accatgccca gctaattttt ttgtaatatc |
| 22801 | agtagagaca gggtttcacc atgttggcca ggatggtctc gatctcctga ccacgtgatc |
| 22861 | cgcccacctt ggccttccaa agtgctggga ttacaggcgt aagccaccgc gcttggccaa |
| 22921 | ttatgcacaa ctttttaagg accatctcta tcacatgaac taaaggatat cattttcact |
| 22981 | tgggggtggg aagtggtgag ctgcttaaaa gcaatgccta aaccccctgg gctttccttc |
| 23041 | ctttcacttg gaagaaccag ggggtaacta actgctaaca attccatgct ttcagagatc |
| 23101 | taggcgcaag cctaagggct tcatctcatt taatcctcat ggcaagactg tacagtatta |
| 23161 | tctccatttt gtaaaatgaa tgataaaaag aacttaagca cagacaggct atagaatcca |
| 23221 | tccaaagaga tggagctgca cttgaggctg ggtctttgag acctgagttt gagcccttca |
| 23281 | tcattgtgtt gtgtgtgctg ctaaccaatt tcccctctct gtcttctagg atttaataca |
| 23341 | tggtgtcaca ttctgtctga tccatcccag tagctctcca gagctcccaa caggagagag |
| 23401 | ttccaaaatg tttccagggg tactaggctc ggttacaata ttttgcttgg tggcccagag |
| 23461 | tataaacgtg gatatcttag gctggtctat agctgaaacg tctatttcat ttgcaagcct |
| 23521 | atctttggct aagaggaagt gaatcattct tgagaacatc taattaattg ctttcagatt |
| 23581 | ccacatgttg acattctcag ggcacatttt ttttttcccg tgcatttctg ttgtcaaagt |
| 23641 | ccctgccagc tcctaaggca gtctgagctg gctgtcttag actttcagag ctgctggaag |
| 23701 | cttgggggag ggaggggctg taggtcaaag aaactttata acctagcttt acctcccagc |
| 23761 | tcagccacca gctgccctca aatgttctgg attggaataa gcccaaagat gagtggcagg |
| 23821 | agggaagggc aagccaatac gtctagtttg gttcagtcaa agccttgccc atttcatcag |
| 23881 | aattttaatg gaaaatttcc aataagatta aaatataagg tcaccccaat tttagtatgg |
| 23941 | ctccatttaa aaaaaatcat gcatatcttt gttttgcaat ggggccttac tcttgcccag |
| 24001 | gagaggtgcg gtggtacaat catggcttac ggcagcctca acctcctgag ctcaagcaat |
| 24061 | cctttcacct tggcctgcca aatagctagg attacagaca cccaccacca tgcccagcta |
| 24121 | atttttaaaa aaaatttttt gtaggtccag tgcagtggct catgcctgta aatctcagca |
| 24181 | ctttgggagg ccgaggcaag cggatcacct gaggtcagga gtttgagacc agcctggcca |
| 24241 | caatggtgaa actgccgtct ctactaaaaa cacaaaaatt agcgaggtgt ggtggtgggc |
| 24301 | acctgtaatc ccagctactc gggagactga agcaggagaa tcaattgaac ctgggaggtg |
| 24361 | gaggttgcag tgagccaagt tcgtgccatt gcactccagc ctgggcaaca agagtgaaac |
| 24421 | tccgtctcaa aagtttttgt ggagatgggg tcttgcgata ttgcccaggc tggtctcaag |
| 24481 | ctcctggata tcaagtgatc ctcctgcttt ggcttcccaa agtgttggga ttacaggcat |
| 24541 | gagccaccac aactgaccaa atcatgcatt tctatagaca acgtctgcaa gaagatatta |
| 24601 | atggtgatta tatctggtta tgttggattt gtatttttat ttgtactttc ctgtattttc |
| 24661 | taaattcctg acattctaca tgtgtactcc tttaataatc agaaaaggaa acttaaaata |
| 24721 | gttaaaacca attggtcaga tatgtaaaat aacccaccat ctctccagag agggctgttt |
| 24781 | gctagaactt attttcttca ttgaaatact agagtgcccc aataagtttg aataacacaa |
| 24841 | aaaaaaagat aatgaaagta actaaattat ctaggcccaa aaggaaatgc cacaaaaatt |
| 24901 | ggcaaagaaa caaccatgac gtgctatacc ggatagctcc taggccccct tggagaccct |
| 24961 | gaggtacccg acgagggacc tgtagtaagg ctggcagaca ggttcttcct ctgttagctc |
| 25021 | tgaggtacaa cagttattct catttttatg tctttcacat ggccagaact ttgcaaatca |
| 25081 | gaggcaaagt gaattcagaa ttaaaaattt tcagcaccat ccaagtcagt aaaacagtct |
| 25141 | tagcttataa cctttatttt ttttattatt ttattatttt tttatttttg gatggagtct |
| 25201 | tgctctgttt ccaggctgga gtgcagtggc atcatctcRg ctcactgcaa cctccgcttc |
| 25261 | ctgagtccaa gtgattctcc tgcctcagcc tcccgagtag ctgggacgac aggcatgcgc |
| 25321 | caccacgccc agccaatttt tgtattttta gtagagacgg ggtttcacca tgttggccaa |
| 25381 | gatggtctca atctcttgac ctcctgatcc tcccgcctcg gcctcccaaa gtgctgggat |
| 25441 | tacaggcgtg agccaccgcg cccagccctt tatttttact gtaagctgcc agtaaacagc |
| 25501 | acacccacct gtctgctgac tgtcccatct ggaagttgtg taggtcctta ctgaatctta |
| 25561 | ccttcttccc ttcccctacc cagaccccat cccagctggc acgtggaatc ctttcccttt |
| 25621 | ctcctcatgt caatagacca gcaggcaaag agagcagtta ccatagtgga aggagaaacg |
| 25681 | ggtcctagtc gtggtgagga ggtagggctg ctgcttaaca Raggccgaga gggacatgtc |
| 25741 | tgatgctcag gtgatccact aggacatctc gcaatactcc catgcccagc tgtaactatg |
| 25801 | atggccaagt ccaagaacca tagcctgata agagtctagg acccccacta tacatgccac |
| 25861 | cagtagaagt gcctgatggg gagagggatg tctagaatga gtaggaggga atgatgacaa |
| 25921 | agcacagcca aggaccaact gcagtggcag gtggcagcca cagattgagt taagtaggat |
| 25981 | tcttgctgtg ttccctagtg gaagcatctc ccttgttgag acccactgag gttaaagttg |
| 26041 | gagacatggg gagaaaacaa tgtccccgtg ttcatcacta gagtcatgat gggcaaggtc |
| 26101 | acaactcatt tgacttcttg gctcccagat tcatatactc tgcctcttgg gggcacttca |
| 26161 | ccataataca gccattggtt cagttgtatg ctgcatcctg agggacagca ctccatcccc |
| 26221 | gcagtgccat gtccaaactg ctgccccgcc tcactctcaa gaggctgtcc cactactctg |
| 26281 | tcaggcacca gcttctgaat gcagtggtaa gactctgtcc tgtgatcacg tctatggctg |
| 26341 | attctctttt gccatactga gtcccctgat caaagcagtg ttatacagcc tggcgcgctg |
| 26401 | gctcatgcct gtgatcccag cactctggga ggcagagggg gatggatcac tagaggtcag |
| 26461 | gagttcaaga ccagcctggt caacatggtg aaaccctgtc tcctctacta aaaattcaaa |
| 26521 | aattagctgg gtgtggcatg agcctgtaat cccagctact cggaaggctg aggcaggaga |
| 26581 | atcacttgca cccaggaggt ggaggttgca gtgatccctg atggtgccgt tgcactccag |
| 26641 | cctgagcgac aagtgtgaaa ctctatcgca aaaaaaaaaa gcaacgctta tgattgatca |
| 26701 | cacactgtac tttctaacct gaaggggtct ggtaaaatca acttgtcttc tcaagggaca |
| 26761 | gtaccaaact gggggctcag cactgacctc tgctggcatg tcggacattt aaaagtggta |
| 26821 | gcagctagag cagtcttgaa gacatggagc cctcactgct ggggccgagc actgctgggt |
| 26881 | tcaattctgc taccaaggct tctccatttc cgagcccatg gtgcaggcac tggggtgcct |
| 26941 | aagaggaagt gcactaactg gacttctgct atgcaccttc tttagggggc attaaagcac |
| 27001 | agttcaaaga ctctcacatt ttgagttaac ccccacaggc ctgtgtgccc cttctccagt |
| 27061 | caggtcatgt tgtcatttga tcctagttat tattctgaaa gggttcagga agggaggtgg |
| 27121 | ggacagatct tgRaaagggc aaacattgcc ttgtatgcct catctgaggc ttccgcatag |
| 27181 | tcttagggga ctgggaaccg tcttaagtga atctcacctt cttcccttcc cccagccagg |
| 27241 | ccctagccct actggcatga ggaatcctat ctctttgtcc tcatgccagg agaccagcag |
| 27301 | gcaaagagag cagttaccat actggaagga gaaatgggtc ctggtcatca gaaggaggta |
| 27361 | ctcctcaagg gacagtacct gagggagaca cttccactag ggaacacagc aagaatccta |
| 27421 | cttaactaaa tctacggctg ccgcctgctg ctacagttgg tccttggctg tactttgtag |
| 27481 | tacaaggcga agtaggccac tgctgccagc ccaggaggtc aagggcattt acccagacat |
| 27541 | cctcattccc agtttttggg tcccttatct tccctctcag gatctaatga ctcatctact |
| 27601 | ggcactagga gtccaaaatg accagatggc agactcgcct aggctgagaa ttcttccttc |
| 27661 | tctcaaattc tgccactttt accagcccag tctgcctctg ctgtaggaga tgagaggcta |
| 27721 | aatgttggca gggaggcccc ctgattctga tcctgtgttt taaatcgata attagtcYga |
| 27781 | ccctgttact ttctcccttc agtgcatcaa cgggaggctc ttagcaacag cctcccaact |
| 27841 | ctaccttcct gatagataat attttcctca aagctctcaa atgctagaga cattgcacca |
| 27901 | gctggYgtgt ccccatccac ctgtatctga tcccaggtca ccacaggtga aggtctgagc |
| 27961 | aatcgaactg ctacagcagg ccaggtacca ggactctcaR cacgccactt aacaccaata |
| 28021 | atgaggtcct catggccatt tggcctgcag tgagccaaca ccagaatccc atccattctg |
| 28081 | ggacccctcc tgctactaac ggtcttcagt caggttcccc agaagcagat tatgcaaatg |
| 28141 | tgttattgaa aaacggcttt cagatgaaac ctggaagaca gtgaaggccg aagctagggc |
| 28201 | agggaagaag ccaggcacag acgtgggctt agcagaagtc tagcatcagc ctgatcccct |
| 28261 | gggcagtact ggagcatgga tggcaccaca ggttaccatc ttgagacaga aggactggct |
| 28321 | tctgtaccct gaatcagtaa gccattggtg ggccataagc cacccttagg ggaggacata |
| 28381 | acctcacagg catttcctgg ctggacgacc ctgggcagct gagggcaact gcctgaaggg |
| 28441 | cacaacggtg agccattgtc agctaacctc acagcagcag agacatgggg ccaacagcct |
| 28501 | ataaagaggg tctgggcagg ctgtcaatac tctctactgt aatactgtat atgtggttta |
| 28561 | ttttaaaaac ttttttagaa ggctttattt tattttattt tttgagaagg agtttcgccc |
| 28621 | ttgttgccca ggctggagtg caatggcgcg atcttgactc accacaatct ctgcctcctg |
| 28681 | ggttcaagga attctcctgc ctcagcctcc cgagtagctg ggattacagg cgtgtaccac |
| 28741 | catgcccggc taattttttg tatttttagt ttcatcatgt tggcctggct agtcttcaac |
| 28801 | tcctgacctc aggtgatccg cccacctcgg cctcccaaag tgctgggatt acaggcgtga |
| 28861 | gccaccgcac ccggctgact ttattttttt agagcagttt taggttcaca gaaaaactga |
| 28921 | gaggaaggta caaagatttc ccatatatcc cctgtgctca cacatgcatg gcctccctat |
| 28981 | caccatcccc caccagagag gcacatttgt tacaatggat gaacgtatac actgaatata |
| 29041 | tcatattcac ccaaagtctg tagctcacat catggttcac tttggctgtt gtacattcct |
| 29101 | tggatctaga cacttttata atgacaggta cagtagtccc cctttatcct caggggctac |
| 29161 | ttccaagatc cccagtggat gcccgaaacc gcagagagtg ccaaacttga ctgccatcag |
| 29221 | tgggaatatg tttctattcg ccttccacca ccaccggttt aacgcctttt tcatcttagt |
| 29281 | gctgctgccg taactttggc agtttgagat gcgacagcaa aatgagtaca aatttctttc |
| 29341 | tccttcttca caatgtcatg gacagatgat tccttcttac catagatctt agcaacctcg |
| 29401 | gtgtgtgatt ttttttcttt cttgttaaat caactttcac cttttcactt aaaggaagca |
| 29461 | tttgacggct tctctttggc atatctgaat ttccagcatc acgactgtgc tttggggcca |
| 29521 | ttgtttgttt atttatttat ttattttatt tatttttttg agacagagtc tcgctctgtt |
| 29581 | gcccaggctg gagtgcagtg gcgtgatctc ggctcactgc aagctccgcc tcccaggttg |
| 29641 | acgccattct cctgcctcag cctcccgagt agctgggact acaggcgccc accaccaagc |
| 29701 | gcagctaatt tttttttttt tttgtatttt tagtagagat ggggtttcac tgtgttagcc |
| 29761 | agggtgatct cgatctcctg acctcgtgat cctcccgcct cagcctccca aagtgccggg |
| 29821 | attacaggca tgagtatttt atttatttat ttattttttc agacagagtc tcactctgtc |
| 29881 | ggccaggctg gagtgcagtg gcaccatctc gctcactgca acctccgcct cccaggttca |
| 29941 | agcaattctc tgcctcaaac tccggagtag ctagaattac aggtgcacac caccacgccc |
| 30001 | ggctRatttt tgtattttta gtagagacaa ggtttcacca tcttggccag gctggtctta |
| 30061 | aactcctgac ctcaggtgat ccacccacct cggcctccca aagtgctggg attataggcg |
| 30121 | tgagccaccg tgcttggcct tggggccatt attaagtaaa ataagagtca cttgaacaca |
| 30181 | aacactgtga tcctaacagt cgatttaatc accaagatgg ctataagtga ctaaggctgg |
| 30241 | cggggtgggg agcacagaca gcagggacac cctggacaag gggataattc gtgtaccaag |
| 30301 | caagacacag cggaaggcgc cagatttcat cgcactactc agaatggcat atcatttaaa |
| 30361 | actcatcgat tgtttatttc tgtaattttt ccatttgata tttggacagc agttgactaa |
| 30421 | gagtaactaa aacctggaaa gtgaaacagt ggataagggg gtgctcctgt acttctcaat |
| 30481 | gtgagacatt tgcccttcat gttaattctg ccccattagY tctacacaaa tgaatagcag |
| 30541 | gaaattgatt ttaaaccaca tggtggtaaa atgctttctt ttttctccct catttaacta |
| 30601 | aagaagggct gggccctagg tgatgtcttc cttagcatct aagcagctgg catcacccca |
| 30661 | ctgcttctgt gctccactct cccatgggac cctccctacc tttaatccct cctgtgctgg |
| 30721 | aggccgggtc ttccttgcta gtggtagctc tttccatatt tttaatccat ggtccggatc |
| 30781 | ctgctccact gcctttgctt tagagaaata aacatgaata ttgagtcact ggaaggaatg |
| 30841 | acacacgcat ccctccccca ccagttggag taacgctggc ccacctagtg tatctctggt |
| 30901 | ctaggtctcg aggacctgct gctcctccct cacctgtagt tgaagacctg cctcaggtca |
| 30961 | gtaggtgata cgcagtaagg aaatgtccaa aggacacttc ttgttggatt acacagcaaa |
| 31021 | catctaattg gctgcaaatc tttttttctt tttctttctt tttttttttg agacagagtc |
| 31081 | tcgctctgtt gcccaggctg gagtgcagtg gcgcaatctt ggctcactgc aagctctgcc |
| 31141 | acctgggttg acgccattct cctgcctcag cctcccaagt agctgggact acatgcgtgt |
| 31201 | gccaccacac ccagctaatt tttgtatttt tagtagagac gaggtttcac catgttggcc |
| 31261 | aggatggtct tgatctcttg accttgtgat ccgcccgcct tggcctccca aagtgctggg |
| 31321 | attacatggc tacaaatctt aaagggggaa gagatgagga ggaataatcc cctttgtctt |
| 31381 | ctcaaaaatg ttttcactgg ctcactacag ctccctcctt cctctttact gacccaaaat |
| 31441 | gccaactatt atagtaactc ttttgggtta gacgaatcca gtgaataaac acctactaag |
| 31501 | cattggaggt ccaagaggaa taagatatgc ctgagctaaa cctcatcacc cccggccttg |
| 31561 | cttgcagagt ggtttgctgg cctcatctga ttattcaatg agtgctttca tttgtttatt |
| 31621 | cactaaatat ttactgagca cctacaaaag tgcctggccc tggtagatga ggcctgaggg |
| 31681 | aagctaaaac taataagaca aactccatgc cctagaggaa ttcatggtct catagggaga |
| 31741 | aaatgtaaac acatcataaa attatagctt attaaatgct gcaatagagt actccgtaag |
| 31801 | agtgtggggg cagagaggag ggggagaaac agctgctttc tagagccctc accttttcca |
| 31861 | cttctctcat ctttctgggt tagggtctag cgggggttcc atgaggatca ggctaaatga |
| 31921 | gcttagaaaa actaagcaga ctactgtatc agaactggat cacagtagac aggcgttttc |
| 31981 | aacaaacata tattgagtat ccctgagtgc tttggacagg aagaggaatt atagacgaag |
| 32041 | attgtaagtc gcagtaatag atgaggcaag gagcaccagt gaggacttaa ccctgaaaga |
| 32101 | agtgtgagca catcttcctc ccagacaagg gggaataaag aaaggaagat gatcaggaga |
| 32161 | gttctaagtg gaactcagca gccagagggg aagcYggagg aggtaacatc agagggtgcg |
| 32221 | tttgcccact cagtaaagta ggaggcaggg cagccttgtg aaaataggaa tggaatagaa |
| 32281 | agctcaagaa aacagccaaa agcaagggca attaggggag gtttcgaact ggcagatcta |
| 32341 | cctcaactgg caatacagca agcatgcacc agaaaagata tcctagctag cagtggggtt |
| 32401 | gggaactgat ttaatatcct aggctagcag tggggttgSg aactgattta ttatttaatt |
| 32461 | ttttatatct attcatgtat ttatgcactt atttattttt gagataggct ctcgctctgt |
| 32521 | cacagaggct agagtgcagt gatgtgatct aggctcactg cagctttgac ctcctgggct |
| 32581 | caagtgatcc tcccacctca gcctcctgag tagctgggac tacaagcacg ccaccaccta |
| 32641 | ggctaatttt tgtatttttt tttgttagag acagggtttc accatgttgc ccaggctggt |
| 32701 | ctcaaactcc tgggctcaag cgatccatct gccttggcct cccaaagtgc tgagattaca |
| 32761 | ggcgtgagcc actgcgccca gccaatattt tttaaaaatt ggaaatccct tgtaataagc |
| 32821 | caagtgttgg gggaagaaaa gcaataaaag caatgacatg gactcaatat gaaacatcca |
| 32881 | aagcatttga catgccttta aaataaaaaa atcagtactc acctcgtgct catttcaaac |
| 32941 | tgtggatttc cttggtctaa aattttaaaa aacaaaaaac agcactctca aatttaagct |
| 33001 | aatttgaatt gattttacat agtgattttt aaatacacat atatgcataa agagaatact |
| 33061 | ataaaaatat ataaaggtgt gtaggtcttg atcatatttt cccaggaatt cagaaaacgc |
| 33121 | tgctacagtc cctgggctca cgtggtcctg gcatcctccc caacgtctct accccatcct |
| 33181 | gctgagtttc tggcatgcat tggttctggg tctgtccaag tttctactgt tagctccatt |
| 33241 | aatactgaat taaggaataa acagtgctca aatgctcatt ttttccatgt gagcccaaat |
| 33301 | tatgtgactt gatgagggaa aaaatcatga gtccctggga gagcgataag aatcacattc |
| 33361 | tttactaaag tgttgtccca ggtatgagaa taacaccaga tctcaacctc cagaggcccc |
| 33421 | ccactgcctc ccacagcata aaagccaaat tcctcggccc gatatttgag gccatctcaa |
| 33481 | tctttttcct ttcaccctat acctgactct cccaggctag gctcagatcg tcactgaatg |
| 33541 | tgctcacttc gaggcacctc tgtgattttc aaggttcctg ggcccacctt ttactaccga |
| 33601 | tgtgactgcc acatgttgcc cagttgctag gatgggaccg tggccttgat ttctgccggg |
| 33661 | actgaggctt tgccttgttt cccacccacc ctgctgcctg cccctgcact cttctggctg |
| 33721 | gggcctgatc tttccccgca gtctcccttc acctctagat cacaggcagt catgccacag |
| 33781 | ctgaggagct tgtcccaaac ctcagtgcct gcctccctcc tcggcttctt gtgctgctgt |
| 33841 | gtctcaccca tcagtgaYgc tcttctcttc cctgcccaag ccctagctga ctccataaca |
| 33901 | cctggataca atgtcctctt ctgtctggtt acctccgaga ggccctctct ctacatccct |
| 33961 | cattggctcc cagtgtcatt cctctcaccc atggccctaa ggtcactgta ttctttggcc |
| 34021 | agtgtacagg gttattatgc ttaacaatcc acaaaggttg aaaggtgttg taggatggtg |
| 34081 | taaaaatgaa tctgggtggt aatgtttata tgtcagagct ttgtaaagtg ctcggcaggc |
| 34141 | gtaaggtact gacagtcctg atattcctga tcttggaacc tgggacacca tcttctcaac |
| 34201 | attgcccgga taccctcaag ggtatccaga cagcctgagt ttgcattctg ttcctggttc |
| 34261 | agcccagggc cctggttccc gctcactcac catccactgt gggccctctc taaattctta |
| 34321 | aagcccttgc catttgcatc actcacagag acatttcatc agagcctact tggtgcacca |
| 34381 | ggctcaggag actcagttct gctgcggata agttatgtaa aattgaccct ctgcttgcac |
| 34441 | atctgtaaaa ggaaggggct ggcacaacac ctctgggcct ttcagttcag tagtgttttc |
| 34501 | tttttatcta aaccacgtgc tgggtcctgg ttttgcttct tatctagatt tttgcattcc |
| 34561 | tgtcacaacc tataaagcac agttcaggcc ttaaggaggg cttgagaaat ctctttccga |
| 34621 | attgtcaact gaataagtgt catatcttca taacaaactt gtcttttttt gcagggccag |
| 34681 | gaaggcagca ggggagtcag ttaaaatata aattttagat taagcttaat attgttaaga |
| 34741 | agtcaattct caccaaattg ttctagagat tttacataat cctaatcaaa atctcaataa |
| 34801 | ggttttttga gaagttggca agcagattct aaaatatata tagaagtata aaggacacag |
| 34861 | aataatcaaa acaactttaa aaaggaagaa caaaattata ggactctaac tacctcattt |
| 34921 | taagacttat tagaaagcag cagtaaccaa gatagtgagg gactgatgtc aaggtagaca |
| 34981 | aatagatcaa tggaaaagaa tcaggcatcc agaaatagat gcacttacat aaatcatcaa |
| 35041 | ttggagaaag aatagtgttt ttaacaactg gcaatggaaa aactagaaca tcagtatgca |
| 35101 | aaggactgaa ctttgatcca tacctcacac cacatacaaa tcaaaaacag aaacaaacaa |
| 35161 | acaaacaaac aaatactcaa aatggatcag agacctaaat gtaaaactat aaaacttctg |
| 35221 | gaagaaaaca cagggagaaa atctttatgg cttttagata acgatttctt aggcaggata |
| 35281 | ccgaaaacat gatacataca gtttttaaaa ttaaatatta taagaattaa agtcttttgc |
| 35341 | tcttcaaaag tcactcttaa gagaaaaaga tgccacacac tagaagaaaa tatttacaaa |
| 35401 | gcattaaaag gacatatatc taggatatac ataaaaactc tcaaaagtca ataataagaa |
| 35461 | aacaatgagc aaaagatttg aacagacact tcaccaaaga agagataaag atggcaaaga |
| 35521 | agcacataaa gagatgctca accattagtt actagggaaa agcaaattaa aacctaatga |
| 35581 | cataccacta tgcccctatt agaaatctga aaatttaaaa gactgacaat accaagtatt |
| 35641 | ggtgaggaca tggcacaagt ggaactctca tacattacta tgggaatgga agatactata |
| 35701 | atcactttgg aaactattta gaaatttcct aaaaaattaa acatacacct accatatggc |
| 35761 | ctaaccatta cactcttagg tatttaccca agagaaatga aaacacatgt ccacacaaag |
| 35821 | acttgtactt gcatgttcat agcagcatta ttcaaaatag ccccaaagca gaaacaaatc |
| 35881 | ggatgttcat taacaagtaa atggataaag aaaacggggt ctagccaaac aatgaaatac |
| 35941 | tactcagcaa caaccaaaat atgtactatt gtttacaaaa tccaaataga tgaatctcag |
| 36001 | aataattatg cagaggagag aagccagacc aaaaaaaaaa gtacatagtg tattatctct |
| 36061 | tatgaaattc tagaaaatgc aaactaacct atagtgacag aaaggagatt ggtggtcacc |
| 36121 | tggggtgggt gagggagggg caggagaaag ggaatgcaaa gcagtgtgaa caaacctttg |
| 36181 | gcggtgatag gcatgttcat tatccttact gtggtgatgc cttacagatg tatacagatg |
| 36241 | tcaaaactta tcagattgta cactttaaat atgtgtggtt tatcgtgtca ttatacctca |
| 36301 | gtaaaggagt tttaaaaatt gtagtaaaag gtctctacct agaaacctta ataagctaaa |
| 36361 | atgtatgtcc ccaggactaa ccctagctat tctatagttc tggggtggaa cctagaaatc |
| 36421 | tgcatttgta gcaagccctt caaataattt gaatgcaggt ggtccttaaa ccaccctttg |
| 36481 | agaaacactg acctagtgag taaggatttc taaacaagct tgtgactaga atgttgtttc |
| 36541 | tgcagggaac aagtgacttt ttctactaga gttgccatat cattctgtgt taagcctcta |
| 36601 | ggacactcct actcaattac catgacatat atttaacata atattaatag ttgtaaaaca |
| 36661 | aaagtaaacc atagtttttt tcccatctta tctgtataca aagtaaaatc aatgacataa |
| 36721 | tattgttact agttcctggc agttacctac agtttaaatc aagaacttat gttgcttggt |
| 36781 | ttttgtgaca gaaaactgat gtggtggctt tccagttaca ctgcttggtt taccttttcc |
| 36841 | cttatcttat gatgcacacc cagctttgca cgttcctgag cagctgatga tcatagaaga |
| 36901 | cctagaaaaa gtgtgagcca gttgcattcc tgctatctct aaagaaaaaa gttatctttt |
| 36961 | catttttatg tgatttttct tacccaaaat gcaaaccact gccagtataa atgatggaag |
| 37021 | atagactttc cacatgaaac tggcatttct tcaccctagt aacattacct gaggggaagg |
| 37081 | tcagaactca attgataaag ctcgctgtgg attttcccac caaatatccc tgcaaacaaa |
| 37141 | gtgaaaggat aaagcctgct taaagatgat ttgtaattgt tgaaaggagt acagtgtgtg |
| 37201 | acatcagtga aaatggtgga gaaaaaaccc gtccccaaat tctcttcttt gtgaaagcat |
| 37261 | ggaaaaaact ggccaaaaat ggttagaaaa tttattttca gaatatcgaa attttttttt |
| 37321 | cctttttttt ttttttatta tactttaagt tctagggtac gtgtgcacaa catgcaggtt |
| 37381 | tgttacatat gtatacatgt gccatgttgg tgtgctgcac ccattaactc atcatttgca |
| 37441 | ttatgtatat cccctaatgc tatctatccc ttccccctcc ccccacccca ccacaggccc |
| 37501 | tggtgtgtga tgttccctac cctgtgacca agtgttctca ttgttcagtt cccacctatg |
| 37561 | agtgagaaca cacggtgttt ggttttctgt ccttgcgata gtttgctcag aatgatggtt |
| 37621 | tccagcttca ccatgtccct acaaaggaca tgaactcatt gttttttatg gctgcatagt |
| 37681 | attccatggt gtatatgtgc cacattttct taatccagtc tatcattgat ggacatttgg |
| 37741 | gttggttcca agtctttgct attgtgaata gtgctgcaat aaacatacgt gtgcatgtat |
| 37801 | ctttatagca gcatgattta taatcctttg ggtatatacc cagtaatggg atggctgggt |
| 37861 | caaacgatat ttgtagttct agatctttga ggaatcgcca cactgtcttc cacaatggtt |
| 37921 | gaactaattt acagtcccac caacagtgta aaagtgttcc tatttctcca catcctctcc |
| 37981 | agcacctgtt gtttcctgac tttttaaaga ttgcctttct aactggtgtg agatgttatc |
| 38041 | tcattgtggt tttgatttgc atttctctga tggccagtga tgatgagcat tttttcatgt |
| 38101 | gtctgttggc tgcataaatg tcttcttttg agaagtgacc gttcatatct tttgcccact |
| 38161 | ttttgatggg gttgattttt ttcttgtaaa tttgtttaag ttctttgtag attctggata |
| 38221 | ttagcccttt gtcagatggg tagattgtaa acattttctc ccattctgta agttgcctgt |
| 38281 | tcactctgat ggtagtttct tttgctgcgc agaaactctt tagtttaatt agatcccact |
| 38341 | tgtcaatttt ggcttttgtt gccattgctt ttggtgtttt agtcatgaag tccttgccca |
| 38401 | tgcctatggc ctgaatggta ttgcctaggt tttcttctag ggtttttatg gttttaggtc |
| 38461 | taacatttaa gtctttaatc catcttgaat tcatttttgt ataaggtgta aggaagggat |
| 38521 | ccagtttcag ctttctacat atggctagcc agttttccca gcactattta ttaaataggg |
| 38581 | aatcctttcc ctatttcttg tttttgtcag gtttgtcaaa gatcagatgg ttgtagatgt |
| 38641 | atgatattat ttctgagggc tctgttctgt tccattggtc tatatctctg tttttggtac |
| 38701 | cagtaccatg ctgttttgat tactgtagcc ttgtagtata gtttgaagtc aggtagcgtg |
| 38761 | atgcctccag ctttgttctt ttggcttagg attatcttga caatgcaagc tcttttttgg |
| 38821 | ttccatatga actttaaagt agtttttttc caattctgtg aagaaagtca ttggtagctt |
| 38881 | gatggggatg gcattgaatc tataaattac cttgggcagt atggccattt tcacgatatt |
| 38941 | gattcttcct atccatgagc atggaatgtt cttccattgg tttgtgtcct cttttatttt |
| 39001 | gttgagcagt ggtttgtagt tctccttgaa gaggtccttc acatcccttg taagttggat |
| 39061 | tcctaggtat tttattctct ttgaagcaat tgtgaatggg agttcactca tgatttggct |
| 39121 | ctctgtttgt ctgttattgg tgtataggaa tgcttgtaat ttttgcacat tgattttgta |
| 39181 | tactgagact ttgctgaagt tgcttatcag cttaaggaga ttttgggctg agacgatggg |
| 39241 | gttttctaaa tatacaatca tgtcatctgc aatttgacaa tttgactttc tcttttccta |
| 39301 | attcaatacc ctttatttct ttctcctgcc tgattgccct ggccagaact tccaacacta |
| 39361 | tgttgagtag gagtggtgag agagggcatc cctgtcttct gccaggtttc aaagggaata |
| 39421 | cttccagttt ttgcccattc agtatgatat tggctgtggg tttgtcataa atagttctca |
| 39481 | ttattttgag atacgtccca tcaataccta gtttattgag agtttttagc atgaagggct |
| 39541 | gttgaatttt gttgaagacc tttactgcat ctattgagat aatcatgtgg tttttgtctt |
| 39601 | cggagaacac tggaaattaa atgatggctt gcagcaatct ggagagcatt tattcaagga |
| 39661 | aaatggctgt gtctcagtat gactaatgag ctttttaact tgccctattt ctatcctccc |
| 39721 | cttccttggt ggtagcctta gaaatgaaca gcctgcaatg atagtgaaaa tcagcagtct |
| 39781 | ggcagccatg ggaggggcag aacaggaatg ggggaactat ggagcctcat tcttagagaa |
| 39841 | ttatcattat ttgatctgtc cacgggtttc taggaatacc tgacctgcaa gtctgtcttt |
| 39901 | attaggcctg actcagaact tgcccaatgt gaaaagtctt ttccccaaag gcctttgtag |
| 39961 | aaaatgatta caggcaattg tttaacttct tggttgctcg aggtattggc taacagtggg |
| 40021 | gcaaacatgg gctaatcaga aggtttaaaa tgaaatgctc aggaataaga tgctcataga |
| 40081 | gggttgtaaa ggctccaaaa tatttatgag actctagaag accatgcaca catttcctgt |
| 40141 | gaacatgttc aggacagatc tgcgaaggcc ccacgatctt acctctggct gatcttgatg |
| 40201 | atctgcacaa acagaaagtg aaagctaggc tagaactgtc aagtgccagg ctgagtgtga |
| 40261 | aggtgtgccc taatgtgcac acagagcccc ttggcaaaga ctagaagact tactgctttc |
| 40321 | aggtgtttaa agaaatctct gtcatgtcat tagtatttag atcactaagc taactgaaca |
| 40381 | gaggcttcaa tggctgcaca taaatacaga cttcacagaa ttagtttaga aaagtcacta |
| 40441 | taacaaacaa caataaacag caacaccaac aaacagtaga ggtgggaagg tccaatttcc |
| 40501 | agagttgcta cattatgtta tttaaaatgt gcaattttaa cagaaaataa tgagacatgt |
| 40561 | aaagaaataa gaaaatgcag tccataccca gggaaaaaga aaaaccagtc aatacaaact |
| 40621 | gttcctaggc caggtgcagt ggctcatgcc tgtaatccca gcactttggg aggccaaggc |
| 40681 | aggcagatca ccagaggtca ggagtttgag accagcctga ccaacatgtt gaaaccccat |
| 40741 | ctctactaaa agaaaaatac aaatttagcc aggcatggtg gcgtgcacct gtaatcccag |
| 40801 | ctactcggga ggctgaggca gaagaattgc ttgaacccag gaggcggagg ttgcagtgag |
| 40861 | ctgagatcat gccattgcac tccagcctgg gctacagagc aagactctgt ctcaaaaaag |
| 40921 | aaaaaaaaag aaaaaagaaa aaactgttcc tgaggaagcc aagatactga ctttactaga |
| 40981 | ccaatacttt aactattttt acatgttcaa aaagttaaag gaaatcatat ataaagacta |
| 41041 | aaggaaagca caagaacaaa tcctcattaa atagaaaata taaagatttg ttttaaagga |
| 41101 | cgaaacagaa attctagatt tcaaaagtat aataattgaa tgagaaattc actagagtgg |
| 41161 | ctggctcaat agcagatctg agcaggcaga agaaagaatt agagaactca gaaataggtc |
| 41221 | aattgaatct atccagtctg aggaagagaa agaaagagga atgaaggaaa aatgaataga |
| 41281 | gcctcagaga ctgtgagata ccttcaagca cgctaatgac gcataatggc agtctcagaa |
| 41341 | ggagagaggg ataaatgggc taaaataata tttaaagaag caatggctga acatttccca |
| 41401 | aatctgatga aaacattaat ctataccttc aagaaagtct ataaactcca agaaatataa |
| 41461 | attcaaagag atcaagatcc acacctagag ataacataat caaactgtca acaaagacaa |
| 41521 | tgaaattatc ttgaaagcag caagcacata gaaggactct tcaataagat taacagctga |
| 41581 | tttctatcag aaatcacaga gttgagaagg caatgggatg acatattcaa agtgcttaaa |
| 41641 | gaaaaagagg gtcaacaagg aattctatac ctaaagccaa tttatcttca acaaagatgc |
| 41701 | caagaccatt caaagYgggg aaagaatagt cttttcaatc aatggttcta ggacaatgga |
| 41761 | tatccacatg taaaaaaagg aacttgggcc cctaattcac atcatataca aaaattaact |
| 41821 | cggaatgagt caaaggctta actgtaagag ttaaaactat aaactctttg caaagaacac |
| 41881 | tatcaagaga gtaaaaagac aatgcacagt atgggaaaaa atacttgcaa ataatatatc |
| 41941 | tgataaaagt ccagtatcca gaatatataa ataactctta caattcaacc ataaaatgac |
| 42001 | aagccaatta aaaaacatac aaatgagtta actgacattt ctacaaagaa gatataccaa |
| 42061 | tggccaatat gcatgttaaa agatgctcaa catcactagc catgagggaa atgtaaatca |
| 42121 | aaatcacaat gagatactag tacatgccca ctaggatggc aatgataata ataataataa |
| 42181 | taataatgtt attataatga acaatacaag tgttagtaag gaaatggaga aaattgaacc |
| 42241 | caactattat attgctggtg tgaatgtgaa atggtggtac tgttttgaaa gatgatttgg |
| 42301 | aagttcctca gaaagttaac aaaagttacc atatggtctg gcaattctac atatatacac |
| 42361 | caaagaaaac tggaaataaa gattcataca aaaatctgta caaaaatatc tacagcagct |
| 42421 | ttacttacag tatccaaaaa ggggaaaaaM cccaaatctt catcaactaa tgaatggata |
| 42481 | aacaaagatg gtatatccat acaatggact gttactcagc cataaaaagg tatgaagtac |
| 42541 | tgatagatac tacaacataa atgaaactta aaaaaatacg caaagtgaaa gaagctagac |
| 42601 | agaaagggcc atatatgtga tgtgtagaga aaacatccaa tagagacagg aaatacatta |
| 42661 | gtgattgcca ggggctgggt aaaggtgaga gatggagaat gattgctaat tggtacagaa |
| 42721 | tttttttgtt gggagagtta tgaaaatatt ttggaagtat aattagaatt aggctgttaa |
| 42781 | atactttaaa acaaaaaaca gctgggcgca gtggctcaca cctgtaatcc cagcactttg |
| 42841 | ggaggccgag gcaggcagat cataaggtca ggagatcaag accatcctgc ccaacatggt |
| 42901 | gacaccctgt ctctactaca aacacacaaa ttagccaggc gtgttggcgc gcctgtagtc |
| 42961 | ccagctattc gggaggctga ggcaggagaa tcacttgaac ctgggaggca gaggttgcag |
| 43021 | tgagcccaga tggtgccact gcactccagc ctgggcgaca agagcaaaaa ctccatctca |
| 43081 | aaacaaaaca aaacaaaaaa caaaagtaag aagtaaaaaa atagaattag actggggatg |
| 43141 | gttattgagc cttgtgaata atctaaaacc cactgaattg tatattttag acagtgaatt |
| 43201 | tgatggcatg ttaattatat cttgatgttt taaaaaaagt agtacagtgt ctgaaaggta |
| 43261 | gcattggcca tttgaatctc tgttaacatt ttcttgggtg gccatcagat ttttgattaa |
| 43321 | aggtgacctt ggcaattaag tggtgggggg aacacaacca aaagggacaa accaacacaa |
| 43381 | ccacacacaa tcacacaacc cctccacctc caccacagtg gaaatcaagc ataggtgctt |
| 43441 | ccagggactg tgaaaatgtg gccacagttg ttttcccagc aaaccatttt ctttcacaat |
| 43501 | atccccaatt tgcacatccc ccactgagct gaaatcagta taactcgaca gacRaaagtc |
| 43561 | agcaggggta acctaccttt gatattattt tgttgttcca tcaataattt acttatttct |
| 43621 | gaaaaccaac agtctctgat ttcttttgaa gctgcctgca atcacacata agaaaacact |
| 43681 | ataagactac catgagtatt aattgcagag tttttattct cccagtgacc gttagactaa |
| 43741 | tgagatagaa aacacttgtc aagtcatggg aacacaataa ggaaagagta gaggagaccc |
| 43801 | atggggaagg catgagcgtt acctgcagga tgtatttctc aagtccattt cgactggcaa |
| 43861 | tctcaaactt tctatggctc ccccttccaa gctggcgaat tgaaagtgtc atcagctatt |
| 43921 | ataaagaggg aagagaaatg ttctatacca tcgttttctg attttaaaat agtccctgtc |
| 43981 | aatcactcta atcctttcta tccttgacag ctctcttctt tcatcttcct ttcaagtttt |
| 44041 | tgctttcctc tcttaatgac tccaagacag cataaaacac aatctggtac agtgtcttct |
| 44101 | gtctcatctg tttcagaagg taacacagac tgttaacttt ctactgtatc tttatgcagg |
| 44161 | aggcgctgca gccctcaacc tttcagtgaa gagatcattt ctccttcaac Rtgtatttta |
| 44221 | caactttcta cgtaatagta actgaataag gatctattca gaaacataaa taacaaggcc |
| 44281 | aggtatggtg gctcatgcct ctgtaatcag tcctgtaatt ccagcacttt gggaggccta |
| 44341 | ggtgggcgga tcacctgagg tcaggagttc gagaccagcc tgaccaacat ggtgaaaccc |
| 44401 | tgtctctact aaaaatacaa aattagctgg gcatgctggt gcgtgcctgt aatcccagct |
| 44461 | acctgggagg ctgaggcaga attgcttgaa cccgggaggt ggaggttgca gttagccaag |
| 44521 | atcatgccat tgcactccag cctgggcaac aagagcaaaa ctctgtcgaa agaaaagaaa |
| 44581 | gaaagaaaga caagaaagaa agacagaaag aaagaaagga aggaaggaag gaaggaagga |
| 44641 | aggaaggaag gaaggaagga aggaaggaag gaagggaggg agggagggag gaaggaaggg |
| 44701 | agggaggaag gaaggaagga aggggagaaa gaaaacaaag gctactatga acaaattaac |
| 44761 | agctttaatc acaaSagcat aaatatatat gggtgtggct cctgtatgtg ggtgtgtgtg |
| 44821 | tttcatagcc tgagagctaa ctggctatga aacagtttct aaacaggtga gaaaacaatg |
| 44881 | cttggtgtgg gcctgggtgc cttagacccc aaggtcagga caatgcctgc acttagacac |
| 44941 | aaagatacac cgttgagaag gRcaagtcta agatgaaatc cagcccatgt tctcatcact |
| 45001 | aagtctttca actctctttt acctctttgc ctggaagaaa gagggtactt ttactaatta |
| 45061 | gtttgtccag actggctgcc atatagtctt ttttctctag tacaaaggca ctcctatgcc |
| 45121 | agccctggcc cataagctct gaccctggag tcagacagac ccggctatgg attctagccc |
| 45181 | tgtggtgtgg tatttggcaa gttaacttca cctctctaca ctgcagcttc ctcatctgta |
| 45241 | aaataaggaa aacgatctct ctcttttctg catagggcga ctcccagatg caataggact |
| 45301 | ccctttacct tcatggYctt cttgaagctg taatgaggag ataatccctg gtccccaggc |
| 45361 | tccattcgta tcttacagaa cactattccc ctttcaaata ggtagatttg cctctggctg |
| 45421 | ggtttaaatc gaatcaaatc cttcatttta taacgatcct tgtgaattgt ccagacgctg |
| 45481 | aaagggccgt gcagcaacag cttgcctagt tttccaatat cgtccttttg gaaacacaca |
| 45541 | tacaggaaaa gaagctgtaa aattacttga cagagaaaga agcctttgtg gcctgttttc |
| 45601 | aaattttata aagcatgctt ctttgaggtt tacaattcta aaagtgctat tcaggctggt |
| 45661 | ctgtcactaa atcaaaacga caattttttg agcttcactg agcgtggtgc cRtttagtta |
| 45721 | tataaggaaa tcaattcagc tatgaaaggt aatgctatac ataaagaatg ttagaaatgc |
| 45781 | ttgagctaga gatacgggga ctgtgatatt tttgactcaa ttttaactag cagaattcaa |
| 45841 | tttcctctgt gctatgtttg atacatgaca gtctccttca aagggacaag aaattaacat |
| 45901 | ttcaattatg tccagtagag atagaatttt ggctgcattg Yggacccatg agcatctgtg |
| 45961 | tgttccatct gcatcctaac ttctgcttca ggtatatgag gacctgagaa aatgaagctt |
| 46021 | gttctgaaaa gagaaagttt actgtgatct aatcttcaca acattgtaat ggaaatagta |
| 46081 | actgcattca actctgagtt tttcagagtg agtcaacagt gccaagataa taccaaatga |
| 46141 | cagattttta agaagtctgt ttgactctag gcataaactg tgatgcccct gtctccacca |
| 46201 | gcaaggctgc ctttgtcata tgtctggttg aaagtccaaa tgagatgacc ctgccttctc |
| 46261 | tgagctctct tcaaaagaca gtggaccaag atgagctggg agcgaggcat tttatctggt |
| 46321 | atttttcaaa acttcgattg cttaaagcag ataagaattc atttccctga ataaaaacac |
| 46381 | caaaaggaac aaattatttg aaggggacca agacaaagaa atctccctaa gtgacctcta |
| 46441 | gctgagatca atccctatga tttctgagtt aatactggaa atatgtactt cattcataat |
| 46501 | tgattgggca tttttataaK ttattgttta tatgttgggc aaactctagg tatctttttt |
| 46561 | aaaaaggaat tgactttact tctgaaacat taaaagaaaa acttcaagag caataaactc |
| 46621 | tagtcatttc tatggtgtct atggcgttat ttcctatgtt aattttctat tttcttcttt |
| 46681 | ggaaaattta catatgtaga ctgtggtgaa taaaaccctc aaactcccag ccacgaggcc |
| 46741 | ttctttccta aaagccaatg ctcctgttct gtgttaacag ttcaccacat tccaaaacag |
| 46801 | aagggtggat aggtccatga ggaccctgga gaattttttc actctatctg catccatcaa |
| 46861 | ggacccactc aaatgtaatc actacagacc ttagtgactg ctttttcctt taaatccatt |
| 46921 | caattcataa tcaatatcga cataaatagt gcaggaaata tgttttagta aaggcaactg |
| 46981 | gtagaagtag atacttaata tttgccacat aaacaaatgt tatcagcaca ggttgagcat |
| 47041 | ccataattta aaaaatctga aatcctccaa aatctggaac tttctgagaa ccagtatgac |
| 47101 | actggaggtg aaaaattcca tacctgacca catgtgatgg gtggcagtca aaacacagtc |
| 47161 | aaaattttgc ttcaYgcaca aaattatttt ttatttttta tttttttgag acggagtctt |
| 47221 | actctgttgc caaggctgca gtagagtggc acgaccttgg ctcaccgcaa cctctgtctc |
| 47281 | ctgagttcaa gggactctcc tgcctcatcc tcccgagtag cttggattac aggcgtgcat |
| 47341 | taccatgccc ggctaatttt tgtattttta gtagagccag ggtttcagca tgttggccag |
| 47401 | gctggtctcg aactcctgac ctctggtgat ctgcccacct cagcctccca aagtgctggg |
| 47461 | attacaggca taagccactg ctcccggcct tcatgcacta aattatttaa aatatgtaaa |
| 47521 | attaccttca gtctatgtgt ataaggtgtc atgaaacata aatgaatttc gtgtttacac |
| 47581 | ttgggttcca tctgcaacat aactcagtac gtatgcaaat actccaatcc tcccctaaaa |
| 47641 | aaaaaaaaac tccaaaacac ttttggcctc aagcattttg gctaagggat attcaaactt |
| 47701 | gtagtttcta tcttacaatg tattcactct ctacctcatt ttaaacattc gacgcatctt |
| 47761 | acagaaatat atacagtata gacagataga aaataaccga gggaattaag gtgaagaaaa |
| 47821 | aataaagaag ttgggagctt ggcgcctgcc tgtagtcgca actacttggg aggctgaggc |
| 47881 | aggaggatca cttgagtcca ggagtttgag gccagcctgg gcaacatagg gagatcacca |
| 47941 | tccctaaata taaagacaat aaatcagatg aactgttcca gaaggtgtag aagataaaag |
| 48001 | aaaagatgaa gaggttatat gtaaaatgca tataataaag ccatKtaaac ttgatgtaaa |
| 48061 | cttggctctg actttctaat agcaatacag agggaaacat ggtgagtggt acgatttatt |
| 48121 | atgtctgaag acatacacaa accggttgtt caggaaaagc acagctattc ctactaagac |
| 48181 | ataagaaaca tttctcccag tgatatgttg tcactgagca acatcctcaa ccacgcccta |
| 48241 | tgatagaaag aggaacatgt tttaggtaaa gctgcttgta agaacacatt tggtcagtgt |
| 48301 | ggaggctcag ctgtcaaagt aattctagaa gaggctagga tgatagggga agggggaata |
| 48361 | agtcgaggtg tacactctct aatgacttgt gaagatgggg tgacgtgagt taaggcatcc |
| 48421 | acaaggctat tgtagaggaa acagccccaa ccagaaatgc tgaagaggca gcctcaggca |
| 48481 | gcaaggtttg tgctttatta cctctgaact ttcctcatca ccccatccac agccatgctg |
| 48541 | ctgcaccttc ataggcaagt aatcagagga tgtgaatcca gagaggtacc agcagctgac |
| 48601 | catagggccc ccagcataaa aacaatgtgc cagggKtggg ggttggcaat gtgatctctc |
| 48661 | cctgaactga agaaaacagg aggaagtgRt gaccagcagg agaYggggct gtgagatgct |
| 48721 | ttaggggaaa gcaggcagtg gaataaacga gaaggaaaca gaactatgaa gaaggtgagt |
| 48781 | cattagaaat tatgaggaag aggaagaaac tgacggaagg gaaaaaaaca gtcagtaggt |
| 48841 | ggtaagagSR ggggcgagca ggcatgtggg tgcacttgac atatacaggc gggtcacaca |
| 48901 | ggtaagaacc ccggctccct ccccaggccc aggccccaga ggtgctgtgg atcctgggtt |
| 48961 | tgctcagttc ccatctccgc gaacttggct ttgccatcgt gacttcttga gaagctgcag |
| 49021 | gtcagctttc cagtaagccc attattactg gaagtgactc ggggggagtc gctgggtctt |
| 49081 | tcaagtctgg cagaaacata tgggcacata tcctctgggt catgttgcac aaatgttctc |
| 49141 | aggaccacac atatgacggt attgcccatc tccacgccca aggtccgatc tccagcgggt |
| 49201 | gagcacatgc cctacacggt gctgaccaag ggcaggcaca cacactgttc actgtgctgt |
| 49261 | gtggagctgc tccgcctcca acaggaagct gcagggacat tccctgagca ggagcaagaa |
| 49321 | tgaccttttc cgtcacgtgt accttttctt tccaactaga ctgtagttga tctcaaggtc |
| 49381 | ggagaccacg ttgcacaacc tctttcagat acgtgttcac cagtatttat ttatttattg |
| 49441 | gcattcccta gtatttattt atttattggc atttagtggt gcttgggagt aggggacagg |
| 49501 | aaacaaaata ctcatcactg tgagggaata ttgatttatc caaggagttc attcagcgac |
| 49561 | gcccctgtac tgggttgaat agtgttcccc caaaaactca tgtctatgag accctcaaaa |
| 49621 | tatgatctta ttcggaaaga gggtctttgc agatacaatt ggttaagatg gggttgccct |
| 49681 | gggttaggat gggtccaaat ccaatgactg gtatccttat aaaaaaagaa aacaaagaaa |
| 49741 | tggagacgca cgctgaggga acagccatgc aaaggcagag gcagaaattg gagtggcaca |
| 49801 | gctgtcagcc aaggaactcc aagaattgcc ggcagtcacc agaagctcag acgaggtaag |
| 49861 | gaaggattct tcctgagagc cctcagaggg aacgtgggcc tgctctcacc ttgcctttgg |
| 49921 | acgtctagcc Ktcaaaactg tgagggaata aatttctgtt gttttaagtc acgtactttg |
| 49981 | tggttccttg cgacatcagc cctaggaaac gaatatactt ccaactgttt tccaccagta |
| 50041 | acaaagaagt gtggccatgt cacacggtgt gtggaagaac ctgatggttg tgaactcaga |
| 50101 | gatggaacat agcggctacc tcaaatgagc gagcgataag ttagatggcc tcctctgtac |
| 50161 | aagaagtgat agcaaaatca attagtaaat gctaccactt tcacacctgg gcattctgca |
| 50221 | ggtaagagcc ccgatacaag gatttcaaat gtgactacat tggccaaatg aagagtctgg |
| 50281 | ctgagagagt gaatctaggg aagctggcct cagcaagagc ttcttttggg tgcccgtgtt |
| 50341 | tccattccag gtagtaagag ttaatatgac tgtcacaaac ttactgcaca aagaagcagg |
| 50401 | aaacatctgt ataaatgcgt ccacacagag cctgagtcca gaaatgaaaa gcagaagctg |
| 50461 | tgggagcctg tgattacaat agtcaggggc aaagaggtat cttcctttcc agcttgagca |
| 50521 | ggtccccata taacgtacca ctagggctgt ttattcggca tctgttgggc gccaggcaca |
| 50581 | gtgcttggtg ccttacccat catttaattc tcacttactc ccggcaaccg cctgtaatat |
| 50641 | aggtattgtg ggggggtggg gggggtgggg gggtggggaa tgaagaagat actttgcctg |
| 50701 | ggtcactagg attatttact tagccctatc tctgcagcta acactgtggt aaggaaagag |
| 50761 | gaaactgaaa tagaaataaa atcctggccc tagctcacaa tgaacttaca atacaagtac |
| 50821 | atcctttagt ggtttatata aatttgttcc aaaaaggaag actgtttcat tcaatcttta |
| 50881 | caagtcaact ttggaactta tccctggcaa gtcagaattt aaatgcatct ctaatatgca |
| 50941 | ttggatttga aaaaaaaaaa aacttttgtt ttttttttgc aaatagatag aagtaaatta |
| 51001 | taactcagtg ttccacaaaa caaacttgtt tggaaaagaa ttttaaactt ctgtgattaa |
| 51061 | attagaccca ggactaattg agtacacaga aaaacaagaa aatataaata tcagaaggga |
| 51121 | tttttttcta gttgctttct agttgtttaa ttgtgataat ttaattatca cattcaattt |
| 51181 | ctttccttta taaaaccagg ttaatattaa atttcaagca gagaaactaa gaggttaaac |
| 51241 | tggactcagc catgcatata gctgtaattt attacaggtg atcataaaca cctgtaataa |
| 51301 | aactctacaa aatggagaca aagaatctca gaatcatttg agctgagtta gaggcagttg |
| 51361 | tcagtcaatt ctaatcagaa acagataaag agtggaatag caccctgata gggaatgaac |
| 51421 | aagaaatagt ccaaggtgac agccatgtaa tcttgagtga gttaactctt ctttccatgg |
| 51481 | atcttctttt gaatcagaag gttgaaaatN ccaatatcta ctcctNcata attcaacagg |
| 51541 | attatacaaa actgtcagga gaaattagct gatgtattag atgtaaaagt aagttaagtc |
| 51601 | ttagtagaaa aggtaccaaa aagcatctgg cagaggcaca attctgagga cacatggtca |
| 51661 | aaaaaggtcc tttgcttttt gcgtgtaaaa tctccaaatt ctgtaagctg gttcatccta |
| 51721 | tttgcatgca aactggctgg gaatgaaatt agggcagRat gttatttgct catgttttaa |
| 51781 | ccattctttt ctcacagcct cctcctccta tgttatttac actgtttcct gccctgtgta |
| 51841 | ttgtttccag gacattcatt agattcaggg aaatgaaatt taatagggat gtctaatacg |
| 51901 | taattcaaga tttaaaaagg aacagaaaga tgccctggat tgacctaaca aattgttccc |
| 51961 | ttgactttcc tcagScgaag aaagaagaat tattaccaag aaaatgatcc ttatacacac |
| 52021 | cctaacccta ctctgcagtt tatgcatatc ctcttgaatc atgcatcagt tgtcatcaga |
| 52081 | gacaccttgg agtcccaggg gacagtaaca gcatcatggc tgggatcaca gcaaacccca |
| 52141 | tggggctaag tttctgagat ccaggcccaa gaaataccag cgtgggaagt aaaaataact |
| 52201 | catttttcgg accttaataa ctttcatggt ttgatttatg acaaggaaag aaaagtcatt |
| 52261 | tcctctacca agacctcaat acatggtatg ttcaaaacca aataaaaatg aaaagaaaaa |
| 52321 | aaaagagaag gaaaaagtat aatataatca caagtgacaa aaacgcagaa gagggggtca |
| 52381 | cagagatgtg atgtaagaaa aacttggccg ggcacgatgg ctctcacctg taatcccagc |
| 52441 | actttgggag gcggagatgg gtggatcacg aggtcaggag ttcgagacca gcctgaccaa |
| 52501 | catggagaag ccccgtctct actaaaaata caaaaaaaaa aaaaaattag caaggtgtgg |
| 52561 | tggcaagtgc ctgtagtccc agctactcag gaggctggga caggagaatc gcttgaacct |
| 52621 | ggcgggcaga ggttgcagtg agctgagatc atgccattac actccagcct gggcgacaga |
| 52681 | gcaagactct gtctcaaaaa caaacaaaca aaaagaagaa gaacccaacc tgccattgct |
| 52741 | ggtttgtgct ttattacctc tgctcataga agccatgagc catggagcgt gggtggcctt |
| 52801 | tttaagctgg aaaattccag gagacagatg ctagactcca aaaaggaaag gaaacacctt |
| 52861 | gattttagcc catgagactg tgttgaactt ctgaccttgg aacttcaaaa taataataat |
| 52921 | aatgaatttg tgctgctcta agccaccaag cttatggtaa tttattatga cagtgaaata |
| 52981 | aaacaaacac atctgaataa aaatctagag tacttcaaag taaaaaacaa aacaagtcga |
| 53041 | atattaacta tgctccagtc tcaaagagat catacagtta taggggatgt tttgcctgga |
| 53101 | atgcctggga ccagcctgct ccggattaac actgacgctg gagctcaact ccccagcatt |
| 53161 | ggcctctaac tgcatctctt gggttttctt tagactattt ggattaatgc atgtacacta |
| 53221 | tatacttttg gggcctcttc tgggatctgt gatataaaat tggggcaaga atataggacc |
| 53281 | tgtttccaaa atacttttct tagagaaact ctgaaacaag aagacaaatc ttttactatt |
| 53341 | ttaatagtgc catgagttag aaaataaatt gctcctccag gacttttcct ctgcccatat |
| 53401 | atgttctggc ccttgacttt ctggtttgtc cagacagtgg gtcagtccca cattcagaac |
| 53461 | cctgaggaat aaatYcaaga ttcctgaaga tgccaaggga cagcttggaa tcttgtagtg |
| 53521 | cgggtttact gagcaattta ggcgactgtc ctcagagtcc aattccttta ccatagacga |
| 53581 | tctctctgac tttgccagag catttcctca ctgtgtagta ttgcacctgc cttttgtgtg |
| 53641 | agatgctcta acactactat ggaacatacc ggacattcag tcactgctgc taggtccaca |
| 53701 | gccaactcac aggacttgat caaatcctct atcactgcca aagcttgttg tagttcagtt |
| 53761 | gagaatgctg aatctttggt tctctttggc ccatcctgtg gaaaacaaat gccttgttgg |
| 53821 | aaatcaaaag tataccatct gcaaagtgaa gaaggaaaac aggacagaga tgaggagcca |
| 53881 | gggatccagc caaccctgac tgagaagggt ggatattgat ctgtctccct gctcccaaag |
| 53941 | catggtggct aaagatgctt actgatttag gtatacggca tagagacaag caaaatcatc |
| 54001 | tacctctgtg cattgtacta gtgacgaatg aagttcataa atcatggaaa ttatattcct |
| 54061 | gctacataag aaacatatag aattcagcta tatagctgaa ttctatatgt ttcttatgtg |
| 54121 | tatatatatt atataaatat ttatatagta tattaaatat ttatatatta atatataatt |
| 54181 | atatgtttat atattaatta tatatattaa atatttatat attatatatt atataaaata |
| 54241 | taagtatata tattatatat atatatatat atatatatat attttttttt tttttttttt |
| 54301 | tttttttttt tttttttgag agcgagagag agtttcactc tgtcacccag gctggagtgc |
| 54361 | agaggcacga tctcggctcg ctgcaacctc tgcctcctgg attcaagcaa ttctcctgtc |
| 54421 | tcagcctccc gagtagctgg gattacaggt gtgcgccacc atgcctggct aatttttgta |
| 54481 | tttttagtag agacggagtt tcgccatgtt gaccaggctg ttctcaaact cctaacctca |
| 54541 | ggtgatccac ccaccttggc ctcctaaagt gctgggatta caggcgtaag ccactgtgcc |
| 54601 | tggccagaat ttagcttttt taaagcatgg aaaaacctag ctccttttaa ataggcttct |
| 54661 | ctctctttta tacccctatt ccctggggct tactgaaaaa aaaaatacca tatggccggg |
| 54721 | cgggtggctc atgcctgtaa tcccagcact ttgggaggct gagacaggca gagcacttga |
| 54781 | ggtcaggagt ttgagcccag cctggctaac atggtgaaac cccgtctcta ctaaaaatac |
| 54841 | aaaaattagc ttagtgtggt ggtgggcgcc tgtaatccca gctacttggg aggttgaggc |
| 54901 | aggagaacag cttgaaccca ggaggcggag gttgcagtga gcccagattg tgtcattgca |
| 54961 | ctccagcctg ggtaacaaga gtgaaacccc gtctcaaaaa aaaaaaaaaa aaaaaaaaaa |
| 55021 | aaagtagctg ggtgtggtgg tgtgtgcctg taatcccagc tactcaggag gctgaggcag |
| 55081 | gaggattgct tgaacacggg aggtggagtt tgcagtgagc caagattgcg ccactgcact |
| 55141 | ccagcctggg caacaaagtg agaccttgtc tcacaaaaaa agaaaaaaga aaaagaaaaa |
| 55201 | gaatacaatc tgagtatcat attcctatat ttaatgtgga agccttattc ctgtgggaaa |
| 55281 | aattatactt aaaatcatcc cagaggagga catttgtaaa ctcctataga gacaaagaac |
| 55321 | tccatagagg ctgctaagtg gaaatttact aatgatttac atgtaaaagc tataacatca |
| 55381 | tatttccaca ctgaatctcc cccaactctg tccttccttc ctccacttgt ccctggccct |
| 55441 | ccccacattg caccaccatt aaagatgcca aagagataag ccagcgctct gcacctcccg |
| 55501 | aacataaaga ctcagcattc agcRgaaaag cagtaactaa ttaaagagac caatgttcca |
| 55561 | attacaacca cagtgacatt taggatgtga ttggggtgat tgtttcagct ctaaaggctt |
| 55621 | ttgcatgggc ttgaggtatt ttatctccct gctacctaca tgctgtattt atctgttacc |
| 55681 | tggtaaatac acataaaaaa attttggttt attcaatgta tttttttaac atctcaagtt |
| 55741 | ctgcagtgaa gaacagctag cccctttgct gctccgcatc tggccctgac tctttttgtc |
| 55801 | ctctacagca caatgtagtc acagggttta atattttctt aatctatgcg gaagcactgg |
| 55861 | gtatttgcat ctttggatga gagacatgag tgacagggct gatgaatgga atagatcgtg |
| 55921 | ctgctgcctc tgaaaaacgt atcatggatt cgtagcttct cattcataat aaagaagctt |
| 55981 | cgcttcctgg taaaagaaaa cagactctca cctgaattta ttttaacaac atcaataagg |
| 56041 | gattaagagt tcttggcagg gcgcagtggc tcacgcctgt aatcccagca ctttgggagg |
| 56101 | ccgagggggg gagtggatca cgaggtcacg agatcgagac cagcctgacc aacgtggtga |
| 56161 | aaccccatct ctactaaaaa caaaaattag ccgggcgtgg tggcgggtgc ctgtaatccc |
| 56221 | agctactcag gaggctgagg caggataata gcttgaaccc gggaggcgga gttgcagtga |
| 56281 | gccgaaactg cgccattgca ctccagtctg ggtgacagag cgagactctg tctcaaaaaa |
| 56341 | ataaaaaaat aagaaataat ttttttttaa agttcttata ccaggcatct tcctccttac |
| 56401 | caaacactga atgctcagtt tcctttcaga tcctcacctc ttcccatccc ccttccttcc |
| 56461 | gctaaccctc cttcacaaaa cacgaggtgg ctagggtttt gaactgtata atgtagggtt |
| 56521 | cagaaaaatc ctttaaaaca ttattaaact cctctaacta gggctagctc ctcttgctcc |
| 56581 | aggctgtaaa aatatgacct gtgtcctgag ctgcttctgt tttcaacagg tccttgtcat |
| 56641 | ccattctgag cagaaagggc atgcaaatga ctgccccata agacatgtct caagtgtttc |
| 56701 | ttgctaaaac cagaatattc tataggaaag ggaagagaaa ccgcactgct ataMcacaga |
| 56761 | tttcttccta acctggtgtg gtcatggtca ccatttattc taagggaact ttggcagact |
| 56821 | cttagagttc acacacacgc acacacacac acagaggaga acaaggcata acatatagaa |
| 56881 | attagatata ctaagaggta caaagaagaa aacataatcc taccattcag gaaagccaca |
| 56941 | gcagacattt tatcatatct atttacttcc agtcttttat gtatgcattt tacgtatttg |
| 57001 | cttaattttg ttcccattaa aatttttttg gctgggtgca gtggctcacg cctgtaatac |
| 57061 | cagcactttg ggaggccgag gcaggtggat catctgaggt caggagttca agaccagcct |
| 57121 | ggccaacatg gcgaaacccc gtctctacta aaaataaaaa aattagctgt gcatggtggc |
| 57181 | gggcacctgt aatcccagct acttgggagg ctgaggcagg agaatcgctt gaacccggga |
| 57241 | ggcagaggtt gcagtgagcc aagattgcac cattgcactc cagcctgtgc aatggagtga |
| 57301 | gactttgtct aaaaaaaaaa aaattatgtg gaaggaagaa aatatattac cacttccatt |
| 57361 | tgggctgcaa atccctactt taaaattgcc tttaattttt ttgttttttt tcttgactgt |
| 57421 | gagttatagg atacatacta ttttagaaaa tctagataat aaagaccacc aaRtaaaatt |
| 57481 | gctataaatc ctccgaMcca tagacaaaca ccattaatag ttagttatat aaaatgtaat |
| 57541 | atttaatatt gagtacctta gccgagcctc ctagttcacc tgggtctatc tccatatctt |
| 57601 | caggagactc gaRatccagc agaccctgca ttaacccaaa agtcttgttg aaaattaaaa |
| 57661 | tgtacattgc catcaaaatg tagattgtca acaaaatcta ctgtgcaaac tatctgccac |
| 57721 | caatgctaag ctgactgaga acgtgtgtaa atgtgtaaga gggaaagaat aaatgatgta |
| 57781 | tttggcacag ggtcctttcc acaaacctag aatcatattg tacacactat tttgtagact |
| 57841 | actttttctc attcaaccat acctcatgaa tattttccca tgatattatg tttttctaaa |
| 57901 | atatgggctt tgattgtggt agagtgttat atcttacaga taaaccattt ttctcattca |
| 57961 | gcaatatctc atgaatattt tcccatgata ttatgttttt ctaaaatatg ggctttgatt |
| 58021 | gtRgtagagt gttatatctt agagataaac catttttctc attcaacaat atctcatgaa |
| 58081 | tattttccca tgatattata tttttttaaa acatgggctt tggttgtggt agagtgttat |
| 58141 | atcttataga taaaccataa tcgaattata gtccccctta tcattaaaca tttaggttca |
| 58201 | acaatatttt ttactataat aatgataata acttttgtgc atttttctta aagctaaatt |
| 58261 | cctagagtgg aattgtaaat gttaagagtt ttgaggcata tttccaagtt cttcagagaa |
| 58321 | ggactgtctt cctgccagct gtataggaaa gcctgtctca ccagccccta gcctacaagg |
| 58381 | ggtagtacca tttaatcaaa aatctttacc aattgaatga aaagagaaat cctacctcac |
| 58441 | tgttgtttta atttgacttc cttagataac tagaggtgta gaactttttt aaaaaaacgt |
| 58501 | ttgctactag tgtttattct tttgcaaatt gctatgtcct ttgcgcatct ttttattata |
| 58561 | atttccaaga gcacatgatt cattaagcat actgatcatc tgttacatat tttgttcaca |
| 58621 | ttttccccaa tgtttcatga gtctgtcttt aatggtataa gctatcactc atcaccctct |
| 58681 | ccccaagatc ccatgatcct ttctaaagca tgaggcaatc agtccaacat tcatgctctt |
| 58741 | tcaagccagc acatgtgtgc gagatgcaaa ataagctctg cccctgggga tagagaaggt |
| 58801 | cctagatagg attacaaggt Ygttcctttc tttgcaggca cgtaatggcc tgagctggtt |
| 58861 | tcacaggcac cagcaagctt agctgtgggg acactgctct ggtctgcctt gggtagctcc |
| 58921 | cacggctcct cacagacccc ccgcaaaata ttttgtagta attctatctc ttgtttcttc |
| 58981 | agtgaaaggc agctaacaaa accttctaag tccttttaac ctgtattatc tcatttaatg |
| 59041 | ctcccaacaa gcataccagg aaagtactat tattatctta tttctatagg tgagaaatct |
| 59101 | gaggctgaga gaggttaagt aacttgcctg agttcacgca gcctggaagg gtcagagcct |
| 59161 | ggattcagac ccaggttgtt aaactctcga gtctgtgttt ccaatgacga tgccccacag |
| 59221 | ccctctgcgt ctggtaccga acctagctcc tatgtaaatg tcacctctgt gggaaagctg |
| 59281 | gaacctaggc tgagggagga aggaccatca ctttcgtcct gctcatgcct cactgggccc |
| 59341 | agaggttgga cttctacaat aggaatgaca gtgacaatgg gacatgcaga ggagcatgag |
| 59401 | gccctgagtg agtgctgggt caaacatgct ctgaggtgct gctcgctgaa agaagcagat |
| 59461 | cattatcctc atttctatgg tggtgtgaca gtctcaatgt ctaagataac agctacacat |
| 59521 | catgcagagc ttcctgggct gtgccaagcc ctccgcatgt gctaatcatc tgcttcataa |
| 59581 | cgatcttatg gaggaggttc tagtaccctc attttacata gaggaaaact aaggcccaag |
| 59641 | aggKcaaggt ctcataactg acaaatggca gagtacaccg aggctgaaac tgctaatgga |
| 59701 | aagcttgaaa ggagtaactt tgaaaaaatg tttttaaaat tatttttaca gatgaggtct |
| 59761 | tgctatgttg cccaggctag tcttgaactc ctggactgaa gggatgcctc ctgcctccac |
| 59821 | ctcctgagta gctgctggga ctacaggtgt gtgttatcat gtctggcctg aaaggggtaa |
| 59881 | ctttttctga ggggagactg ttgggcaatg agcttaacct tcctgagcct cagtttcttc |
| 59941 | acagtccaga gcatggctgg tatgtctgat tgtggagatt aaataagaaa tactgtaatc |
| 60001 | ccagcacttt gggaggctga ggtgggcaga tcacgaggtc aggagttcaa gaccagcctg |
| 60061 | gccaacatgg tgaaaccccg tctctactaa aaatacaaaa attagccagg catggcggcg |
| 60121 | cgtgcctgta atctcagcta cttgggaggc tgaggcagga gaattgcttg aacctgggag |
| 60181 | gcagaggttg cagtgaactg agatcgcgcc attgcactcc agcctaggca gagtctcact |
| 60241 | ctgtctcaaa aaaaaagaaa gaaagaaaga aaaaagaaat acacgtgggg aagtacccag |
| 60301 | taaacagtag gtgcttgttt ttgtttttgt ttttgttttt tattaagaaa tagtggccac |
| 60361 | tgggcaagag ctacctaaat gccttataga ggttaattca acagacaact tatcgtggtg |
| 60421 | gcccaaacct ttctctcaag ctcacaccta gaaaaccagc catgataggc cctcatatcc |
| 60481 | tgtttggagt gtctgagaat gcttgcctgc agatgtgcta gtgtcaatga cttatcacta |
| 60541 | aggaggggta aaggtcagag agagagagag aaaaccactt aggggttcag ggttcagata |
| 60601 | tagcaaagtt cattctgtta ggctgctctc agggtaagat cctaaatcta agaatggagg |
| 60661 | tcagtgctgc aaaaggatag aagacttcag ttttgtcttc tcccttccac aagttaaaat |
| 60721 | gcccataaaa cagtaacttt ggggaaaatc acactctcgc tcccaaagag ctctcttccc |
| 60781 | ctaagccaga ctccttagtg attcatgccc tgagctagac attggactag tgaggccctc |
| 60841 | aggtgccctc caactctatg attctttggt cttactccac attgaagagg gctgatttag |
| 60901 | agttggagga aagaaaaagc ctgcaggata gacgtatctt cagccttaga agaccaaggc |
| 60961 | acagctctac atgtgtaacg acatgggcac cacagtgcag gcgtctggtt tcccaccaag |
| 61021 | gaaaagcact gtctcattga ttttaccttc aacagcatct ggtattttat aagtctctgg |
| 61081 | cttggtcctt tgagatactt aaaaagaggg agattgtgat ccagttggcg ctggcatacc |
| 61141 | tttaaaagcc aaatagaaac aacataaagc aaaacaaaca acacacacag aataaaagtg |
| 61201 | gcagcattta ggtacaaaac acaaaattgc ttttcctgga caacaggggc agctgtgtcg |
| 61261 | agcagctcca taaggctgtg gggttgttct ttcccctagg aacagtgtgt gaaataagag |
| 61321 | aaagaggaaa gaggacggct cggctaatat ttttacaggc atctagggaa gcagaaccat |
| 61381 | ttatttcttt tagaaagcaa gttcactaga agacagtggg gcatttcaag tccttctgaa |
| 61441 | aaagaggcca cctatcttca agttgggggc tgccagagtc tattcaggcc ttttggagcc |
| 61501 | tgaaggatac agacggtttg tagacatcat tccagggagc ccaaagtgtg acatttttta |
| 61561 | cttagtttca tttttaaaac ccatcttaga agaagacact ggttttctca ccagatatag |
| 61621 | gtgctcctca caggcctatc ccattacaac ttttcatgtg taatttcaaa gtatgggaca |
| 61681 | ctttaaataa acataaatcg caatgccaag ttcctctccc tagtcccact tttgatttct |
| 61741 | ttaacaactt gatgcataga aacattttcc aggaagaagt ttgtcttctc aacattaccc |
| 61801 | caaagtaggc gcagtcttga cactcttgcc agattgccct agctcggggc agattcttat |
| 61861 | ggtatttaaa atatatctga agatcttctt tctaggtggg aaaaaattag aataaattaa |
| 61921 | tcaggcagca tttttcagta agtggacagg atgatacagt ttattcattc attcaacaag |
| 61981 | ccagagtatt ctaatgtgta aggaattggg ggtgtacagt aagtaatctt atgttgggat |
| 62041 | gtgtgtgtga tggagagata aacagaaggc tttaaacaac cataactacc agacatagat |
| 62101 | aggcacagag tgctctggga gcatctacaa gggctattgg taaatacata agtagactca |
| 62161 | caaacaggaa ttggttttta tctttatcat tcttttcctg ccctttgtcc aggacatcga |
| 62221 | catctcccca caacaggtct gcaatgagta gtaaaactga cagaaaagag gctggtgttt |
| 62281 | gatggtcccc ctctcttctc tctccaccct ctccttgtgg tcatcgtgac accaaccctg |
| 62341 | caaagaagcc caagaaacaa ccaaaagatg gaaacatgcc agaggggaga tgtggtagat |
| 62401 | cgggcaacca atacggagct cagtcatttg ggcaaccctc aaagtgacag gctccgcctc |
| 62461 | aaacatcact ttcaacctgc agcacaagac tctgagacag gaaatgttat gtgactgttt |
| 62521 | cggagctaga gagcaaggag gactcaacgt gctgttcaac ctgtattctc atggtatcaa |
| 62581 | agacgctgag aggtgggcac atatctgcca atctcccgga gagaaagcac actccaagcc |
| 62641 | tctggcgtca gcttgccagc ttcactcgct ctatcaggga gcctggccca cagcggctgt |
| 62701 | gctggccaca gatcccatgc cattctggtg gagaagccca tgtagccacc tccttgcaga |
| 62761 | agtctgtgtc cctgtcctcc tgctcagccc aactctggat ttccttcatg tcaacacaga |
| 62821 | tgattccttc cctctcagct tcaatgatgc ctgttctact cccaaataca ttgctatagt |
| 62881 | ggatgacttc catttctgta gctctttctt tctcccaata ggttgtaagt ggtcatttca |
| 62941 | gcataggaac tacagcaacc gtcctttgct attccctcca ggaaaaaaat ggcacacaca |
| 63001 | tttaagacag atggcccttg aaaaatgttc agactggtta aggagctaga ataataactc |
| 63061 | agagcttaac aatatagtat tgacctaaat tgctcttcta aaggctaaca gtccaaaatt |
| 63121 | cctcagagaa aggatgtcca ctaaccagca acatacaaaa gtgcccaatt ctcctaatct |
| 63181 | ccttatagaa tgaatcagga ggctataaaa ttgctaccag aaactcatat tcacacaaga |
| 63241 | atacatactt gcttacggat taagactgcg gggctctggt ggcggatggt ccaggttcag |
| 63301 | ttcttggctt tgccaaatac aaatcatatg gcctaaagta agttacctaa ccttcctaga |
| 63361 | cttcagtttc ctcatctgtt aagtgggccc ataacagtag tcaactcctg tgactgttgt |
| 63421 | gagattaaat gagatgatgt atagaaacct ctccacaaat gcccaatgga tagagtactc |
| 63481 | aaaaaatggt ggctattgtc aaagcacttt ttactttatg gaatcctctg catacattat |
| 63541 | tttgtttccc acaacaactg tacatattgg tactattagc ctgttgtgtg gaggaggaaa |
| 63601 | gtgagattcc aaagtttaag ttatttactg gaggtcatgc agatagcaag tggcttgacc |
| 63661 | aacatgataa atcgggtttc ctgacttacc agttactccc acatatcagt gtgagcaccc |
| 63721 | attccagtgc tctttccatt ttaccatgtg agatctcaga gtacttggaa cccacagtac |
| 63781 | actatggaca aatattattc tatatagaac atggtctctg gaggagaatt gtttcagaag |
| 63841 | atacaggcta aagaggctga ttaaaacact tggtcagaaa actatttcaa aattcattat |
| 63901 | cttggaaggc cgaggtgggc ggatcacgag gtcaggagat ggagaccatc ctggccaaca |
| 63961 | tgatgaaacc tcgtctctac taaaaataca aaaattagcc gggcgtggtg gcacatgcct |
| 64021 | gtaatcccag ctacgaggga gggtgaggca ggagaatcgc ttgaatcagg gagtcagagg |
| 64081 | ttgcagtgag ccgagatcac gccacagcac tccagcctgg tgacagagcg agactccgtc |
| 64141 | tcaagaaaaa aaaaaaaagt actacttgta ttttgtctct aacatcataa atcacatagg |
| 64201 | gctaagtcag tgttttctgg ctgggttaat gatttacaat gttcctccca acatggcggg |
| 64261 | gcgctatcaa aaaactttgt aaaactttac aatttaggag aggaattcag aaaaggtatt |
| 64321 | tagttaataa ttctctgaca atttcctcat atttgaacaa tttgataata tccactttcc |
| 64381 | cacaggaact ttgcccattt ctcattgagc aatttaaata atctttgcag taatgacata |
| 64441 | cataaccata aaatacttct aatctctgaa actagatttc acattagtcg tttgttaagt |
| 64501 | gcaaaaattc aaaatagtat ataaagcact aataatgtaa tagttcaaaa aaattaatga |
| 64561 | atcagaatga gtataaaata gttttcagtg tacattgaat tctggtgctt tgacacaaag |
| 64621 | cctatgatga gttgacatgg aaagccatat ggggccacca aaaggctctg agcatggaag |
| 64681 | attccttctc tccattaatg tcaaatatct tgaaacctgg aggctttgtt tgcttccagt |
| 64741 | tatcataagt attgctctca gcctcagtca taatatgagt tgcttacaac tttaagttgt |
| 64801 | ttgatctgtt gccttgggtt cacaaaaata aaaataagaa agagaatgag aaggactagt |
| 64861 | tgagaaagag aggagaaatg agaactattt agaaggcaca gggggtcagg atcatctcca |
| 64921 | tggtcgtgtc ctagaaagta cctggtgggg attgatgttg ggtggaacat ttctctgtgg |
| 64981 | cagctcccac tggcccatat agcctatttg ttgcctaaaa cagggtggag aaacagggaa |
| 65041 | agagggaaga agtctgggag gtgggaagaa ggggtagttg gaggtataat gttgtaggcc |
| 65101 | aagatgttca taaattgagt actaagccta aaggaggctg tagatttaga attttcgaat |
| 65161 | catcctctat gttgtaaaaa atagaatgga aagaaaatgt atttcctgag gtttttagtt |
| 65221 | taacaccagt gcttctcctc ttgagagcat tagtacagct ttattgtggg actctaacct |
| 65281 | cgctgtatga ttcttgtttt aaaacgtagt tgagtcattc agaagaggat cctctgcttt |
| 65341 | ggggaccatg gatggccagc cttccatttc atggggcttt gcaggaagcc aggcttgcgg |
| 65401 | ttactcccac atatcagtgt gagctcagtc aaattggact cattctcacc ttggataaag |
| 65461 | tcagataaag ttgggaggcc aactcatgtt ttccattcaa agggcagata caagatatac |
| 65521 | tgtcttgaca aattgcaagt gtacagtaca gtattattcc atgatagtcg ccatgctgtg |
| 65581 | cgttacatct ccaggactta ctcatctcat aactgcaagt ttgtaccctg tgaccaacat |
| 65641 | ctccctactt cctctagact ccagcccctg gcaaccaccc ttctacttca atgagctcaa |
| 65701 | ctgtttttgg attatatata tatacatata cacacacaca cacacacaat ggtaatgaat |
| 65761 | gtattcatta acttgattgc agtaatcatt tcacagggtg taagtatatc aaaccatcat |
| 65821 | gttgtatagt ttgaatatat gcaattttta tttaccaatt atacttcagt aaagcttgga |
| 65881 | gtcggaggga agcagatatg tttttagacc attgatgttt ccatttgtcc ttgccactta |
| 65941 | acgcagaaat gtaagggctg actttaacat gattatttac tgggagtaca gacttactct |
| 66001 | cttgagaaag caatgtgcca gaagttcagg gttctcagca cacttttcca actcttttag |
| 66061 | aaaagtccta gaaaaataaa agtatacaag taataatgtt ttgattttga catgtaggta |
| 66121 | attaattttt taagtacaat cacataaggt ttcaagaggt gcctagaaaa atgttctcca |
| 66181 | ggccttcttg ctcttatctt ctacacattg attcagRaaa aaaagctgta ttctgaggta |
| 66241 | ttactacaat taccctgaaa tcactcacaa ctttaagttg ttcgatctgt tgccttaggt |
| 66301 | tcacaaaaag aaaaataaga aagagaatga gaagaactag ttgRgaaaga gaggagaaat |
| 66361 | gagaactact cagaaggcac agggggtcag gatcatctcc aattatcaga acacagctgt |
| 66421 | tcgggtaatt ctaagcacta gtcatagata gactggatac attcactgat gctctctctt |
| 66481 | acatgaaagt aacgttcata agaactgctg ctgctgctgt tgctattact aatatcattg |
| 66541 | acagcttact ataagccagg cataagctaa gtgctccgca aatagtatct catttaatcc |
| 66601 | tcaaccaaag atagggtttt ataaatataa aacctggggc tcaaagaggc tacataactt |
| 66661 | ctctggggaa atcaacagaa atgggtttcc atttcagttt tgcgtctgac caacggacga |
| 66721 | ggaatggggt taggaagcaa ctgtggttca attcaccaag cagcttttct ccctctgtga |
| 66781 | attaggtgtg caatcttggg gtcatcatag cgaataaaga gagttaacaa gttaccattc |
| 66841 | ccatatgtgt aacatgaaag ggtttgaaga tgatttccaa agtcccttat tcctgcaatt |
| 66901 | ctctagaact caatatccaa gcagccccga gttaaaagta caacttgttt gagcagtcag |
| 66961 | cattttctaa caccctagtt cccagcatcc ccttatgata gtagtatgga gatgtgtatc |
| 67021 | ttggtcatct ttgtattccc aatgctgagc acagtgcctg gcacagaaca ggtgctcaaa |
| 67081 | aaatgctgat gcatgaataa ataaacaaag gaacactcaa ctgcatacaa catggatgtc |
| 67141 | tgacactggg tccctgttcc tttgatgtct cttccctttc tctccaggga tcctgtgggg |
| 67201 | cttctcccct cctttcactc caacccaccc caatccaatc cactgactcc aaaccacttg |
| 67261 | cttaaacctg aaaacatcta agtgttttca tctctcaatt ccatcagtct catttccacc |
| 67321 | cttctSttac tctccattct ttctgataac atgaattatt atatacctgt tgtgaaattc |
| 67381 | gtaaagttct ctaatattcc caaagagaaa gtccttgtta ttctgaagaa catctggaat |
| 67441 | tagatgcttt agccaaataa aatccattgg agtgatatat ccctgcagag gtgcaaacaa |
| 67501 | ggtaggtgtt acaaacagga acataccaga gatcatctga attagctact gcaaaaccca |
| 67561 | aaactttagt gaaatgattg aagaatatag cacacttgtt tcttgaaagc tatcgtattg |
| 67621 | agtactttct tttgatatca ttttcctcat gtgcccactt tcagaaggag atggcttaat |
| 67681 | ggcaatgatc ataaaatggc atttttgtgg gaagaacaaa tatatttaaa aatgtttatg |
| 67741 | ttacacaggt ttaatctaaa agaagggaaa atgtacccac tgttttatga actaatacaa |
| 67801 | ttactattat tattatgatt attattaata ttgagacgaa gttttgctct tcttcccagg |
| 67861 | ctggagtgca atggcgtgat ctcggctcac tgcaacctcc gcctcctggg ttcaagtgat |
| 67921 | tcttccgttt cagactccca agtagctgga attacaggca cacaccacca cacctggcta |
| 67981 | attttcgtat ttttagtaga gacagggttt caccatgttg gccaggctgg tctcaaactt |
| 68041 | ctgacctcag gcgatccacc cgcctcggcc tcccaaaatg ctgggattac aggcgtgagc |
| 68101 | cactgcatcc ggcccaataa ttatttttta atatctgaat aatgtttact cagcatatat |
| 68161 | atcatagttt actaaacatt ccctttttat ggacatttgg gttgcttcta atattttatg |
| 68221 | gtaaaaatgt gattataaat aatcgcgtac atataatttt ttccttaaga ttattccctt |
| 68281 | aggaaaatag ggaaaacatg catttttact cttaagaaaa agactacttt aaaaaaacaa |
| 68341 | gaaataatct tcccttatta aagtatcagg aacacttata atatcctttg ttggcaagaa |
| 68401 | gatggtaaac ctatttgggc tttgctggaa tcagtgtgaa ctggcttaat tctttggaaa |
| 68461 | ctatttggct acatggatca agaaaccaca gggatgttct tattcctact ggagacactt |
| 68521 | gtaatattct aagacctgaa accttggtgg gaattctgga gacttctggc aactattttg |
| 68581 | agtctctttg tacaacaaaa catctgcagt ttgaatatat gcttgatacc ccactcccca |
| 68641 | aatctactta catcaattat gcttttaatc tcttttatgt aaatctcttc agtctcaagc |
| 68701 | aagtcacgta taatgcgcct gaatcacagc agcaggtggg agggtgaaag agagagagac |
| 68761 | agggagagga gaatgttctt ttagaattct gttagaataa tgctcatcaa tacagttccc |
| 68821 | ttttagtggc tcacctactt agtttctggt cagttcattc tgttctattg aacacccaag |
| 68881 | gtcagcatct cacaaacgca cactgtgatc acactggtat caagaagaaa cagcaaggtt |
| 68941 | agagaactca aaatccttta ggcagccagt gaatgacctg tcctctgggt gggccatata |
| 69001 | gtgtgggggt caagaaggga gatgttggag tcagaaatac caggtctgga tccttagtca |
| 69061 | aggaaggtct gcctgacccc acccccaccc cctatattgt atacattgta taagtggttt |
| 69121 | cctagatttt ctctccagct ttctgctcac atcaagcttt cttttccttt taagagaaag |
| 69181 | ggtcttgccc tgttgcccag gaatgagtgc agtggcatga tcatggctca ctgtagcctt |
| 69241 | gaactcccgg gctccagcaa ccctcctgcc tcagcctccc aagtagctag ggctacagat |
| 69301 | atgcaccatc acacccagct aacgcctttt tttttttttg gtagagatga ggtcttgcta |
| 69361 | tgttacccag gctggtctca aactgctact ttcaagcaat cctcctgcct ttgcctccca |
| 69421 | aactgctggg attataggtg tgagacactg tgccaggcca gatccagatc tttgacccat |
| 69481 | atggatgtat tttgttgtac atggggttag atgtatgcta gctccttcct ctggcattgg |
| 69541 | atgtttcact gtgatttcct aaggcaaaag atatgacttc tgtctgcttc tgtcaaggaa |
| 69601 | tgcaggtgga agatgtggtg gacagaattt aagacggtcc ccaagacttc tggccccact |
| 69661 | gcacatacgc ctcttccaat caaacactaa tttaggggat gctgtggaag gattttgctg |
| 69721 | ttcatttgat aggtaatcca gttgggcctg tcctaatcat atgaactcag atctatMtgg |
| 69781 | gtcaagtggt cagagactgg aagcataaaa aagattcaac acaaaggaga ttcccctttg |
| 69841 | ctgacttttg agatggaggg ggccagatgg aatggaatgt gggcagccgt aggaactgag |
| 69901 | agtggctccc agctgacagg cagcaaggaa gggaaatcag tcctatagtt gcaaggaacc |
| 69961 | gagtcctgcc acaaccacgc gaacttagaa gaggatccag agctccacat gaaaacacag |
| 70021 | ccagccaaca ccttgacttt agccttgtgg gactcctgtc ccagggaaac tgtaggtctc |
| 70081 | ctaagtttgt ggtcagtttt tatatagcaa tagaaaacca gtagaaagag taaggccagt |
| 70141 | ccccacatct atcccagaca gactttcttt ctctcctcag gctacgtggc catatttatt |
| 70201 | atttctttta ggagtccaag tagatgagtg tttatacatg tgtccttgtg tagaatatat |
| 70261 | atatttattc ttgttaacta ttgatggatt atctattaaa tggcagcctc tcctagaaaa |
| 70321 | tgatttgttt ttctctcata aaaatggatg tgacaaatga tccaatagaa aaaaatggga |
| 70381 | aaggacacaa aataggagat tcacacaaat attcaaatgg atatgagact cgtggaaaaa |
| 70441 | atactcagta catacattca acgaaggcat attttaaaag agcaacaatt ttcatctatt |
| 70501 | agaatattaa acatttcaaa cctcatagaa agactggtaa atgagggtgt ggaaaaaccc |
| 70561 | tcacacaata ttggtaaaat gtgtaaattt gtgcaagctc tttggaaggt aatttagaaa |
| 70621 | catctcaccc aaatgtaaaa cacgtgctcc ttggttcagt aatttcactt ctaggaatgt |
| 70681 | tcattgttta taattaggac aatataactt caacaacaac cagatataaa ctggttactt |
| 70741 | aaatgctcat caaaaaatta tggtacatgg gccgggcatg gtggttcacc cctgtaatcc |
| 70801 | cagcactctg ggaggctgag gcaggtggat cacttgaggc caggaaatcg agaccagcct |
| 70861 | ggccaacatg gtgaaaccct gtgtctacta aaaatacaaa aaattagatg ggtgtggtag |
| 70921 | cgcatgcttg taatcccagc tacttgggag ggtgacgcag gataattact cgaacccagg |
| 70981 | aggtggagat tgcagtgagc cgagatcaca ccattgcact ccagcctggg caacaagagt |
| 71041 | gaaactccat ctcaaaaaaa aaatatatat atacatacat atagatatag atagatatat |
| 71101 | gtgtgtgtgt gtgtatatat atatacatat atatggtaca tacatataat gatgaaatac |
| 71161 | catgcaattg ttaaaaagaa tgaggctgac taaaacactg atatgacaga ggccaggtat |
| 71221 | ttttagtgaa aaaacaaaac aagacgtaga caagtaagca tagtatgact atttgtgtaa |
| 71281 | aaaatgaatg tgtatgtaga aatatacgta aaactgtata tgctcagaaa atttatgcaa |
| 71341 | agaaacttaa cacattgtta aaagtgattg cttttggaga gtggtactga aagctaaagt |
| 71401 | atgggaaagg agaatttcta catcttactc tatacccttc tacaatcttt tttaaagtga |
| 71461 | aacatttact gcttttataa tggaaaaata gggtttacat aatattttaa aatgaatgtt |
| 71521 | actggaggta atgatatgta aaccctattt gatagatata aataaataat acaattcaca |
| 71581 | tattttacag ctcattattc tttcttataa tcatttttgt gtctatttat aaatacttgt |
| 71641 | ataagacaag actgataaac aagatgtacc acagaggatg taatttccgg aagtctaaaa |
| 71701 | ctcccaaaaa gttacttaaa catcatactt tactaagtca ctgagaaaga ggttcctatg |
| 71761 | ccttttataa atcacccgga aacaacaacg gtttctatgt cagaaggaag tacaggcctc |
| 71821 | aataccaata tgtttctacg agccctggca tcaagtggga ccacagcaac agttaagcat |
| 71881 | tcatggaaaa cctgagtaca atcagcctct tatgccttca tgaagcactg ggcttaagca |
| 71941 | acttggcaag ctggcttcag gatgcattga caattaaagg aagtcagaag gcagaaataa |
| 72001 | ccaggaggtg gacagaagtc aaggttatta tccaaaaatg atctatgaac ttaagagact |
| 72061 | cttaggagct tttaggactc taatacagga aaatattcac atctctgaag gaaaagaaat |
| 72121 | cccttggtca tattctggat tacagtttgg aaaaaaagtc tagaaatctc atccaactgc |
| 72181 | ttcattttac ctgttggagt gcaagagata aaatgccaag gataggaact ccactgaaga |
| 72241 | atgtaggcaa atcaatattc attaaataca ttcaaattaa tatatgactt aaaaagagcc |
| 72301 | ctcacatcag tgttaggcca ttgcttttcc ttgcttctct tgttttcctt tcaaatacct |
| 72361 | ttataaaata ggagaaaatg tatgactctt tttttttttt tttttttttt attatactct |
| 72421 | aagttttagg gtacatgtgc acattgtgca ggttagttac atatgtatac atgtgccatg |
| 72481 | ctggtgcgct gcacccacta atgtgtcatc tagcattagg tatatctccc aatgctatcc |
| 72541 | ctcccctctc ccccgacccc accacagtcc ccagagtgtg atattcccct tcctgtgtcc |
| 72601 | atgtgatctc attgttcaat tcccacctat gagtgagaat atgcggtgtt tggttttttg |
| 72661 | ttcttgcgat agtttactga gaatgatggt ttccaatttc atccatgtcc ctacaaagga |
| 72721 | tatgaactca tcatttttta tggctgcata gtattccatg gtgtatatgt gccacatttt |
| 72781 | cttaatccag tctatcattg ttggacattt gggttggttc caagtctttg ctattgtgaa |
| 72841 | tagtgccaca ataaacatac gtgtgcatgt gtctttatag cagcatgatt tataatcctt |
| 72901 | tgggtatata cccagtaatg ggatggctgg gtcaaatggt atttctagtt ctagatccct |
| 72961 | gaggaatcgc cacactgact tccacaatgg ttgaactagt ttacagtccc accaacagtg |
| 73021 | taaaagtgtt cctatttctc cgcatcctct ccagcacctg ttgtttcctg actttttaat |
| 73081 | gattgccatt ctaactggtg tgagatgata tctcataatg gttttgattt gcatttctct |
| 73141 | gatggccagt gatgatgagc atttcttcat gtgttttttg gctgcataaa tgtcttcttt |
| 73201 | tgagaagtgt ctgttcatgt ccttcgccca ctttttgatg gggttgtttg tttttttctt |
| 73261 | gtaaatttgt ttgagttcat tgtagattct ggatattagc cctttgtcag atgagtaggt |
| 73321 | tgcgaaaatt ttctcccatg ttgtaagttg cctgttcact ctgatggtag tttcttctgc |
| 73381 | tgtgcagaag ctctttagtt taattagatc ccatttgtca attttgtctt ttgttgccat |
| 73441 | tgcttttggt gttttggaca tgaagtcctt gcccacgcct atgtcctgaa tggtaatgcc |
| 73501 | taggttttct tctagggttt ttatggtttt aggtttaacg tttaaatctt taatccatct |
| 73561 | tgaattgatt tttgtataag gtgtaaggaa gggatccagt ttcagctttc tacatatggc |
| 73621 | tagccagttt tcccagcacc atttattaaa tagggaatcc tttccccatt gcttgttttt |
| 73681 | ctcaggtttg tcaaagatca gatagttgta gatatgcggc attatttctg agggctctgt |
| 73741 | tctgttccat tgatctatat ctctgttttg gtaccagtac catgctgttt tggttactgt |
| 73801 | agccttgtag tatagtttga agtcaggtag tgtgatgcct ccagctttgt tcttttggct |
| 73861 | taggattgac ttggcgatgc gggctctttt ttggttccat atgaacttta aagtagtttt |
| 73921 | ttccaattct gtgaagaaag tcattggtag cttgatgggg atggcattga atctgtaaat |
| 73981 | taccttgggc agtatggcca ttttcacgat attgattctt cctacccatg agcatggaat |
| 74041 | gttcttccat ttgtttgtgt cctcttttat ttccttgagc agtggtttgt agttctcctt |
| 74101 | gaagaggtcc ttcacatccc ttgtaagttg gattcctagg tattttattc tctttgaagc |
| 74161 | aattgtgaat gggagttcac ccatgatttg gctctctgtt tgtctgttgt tggtgtataa |
| 74221 | gaatgcttgt gatttttgta cattgatttt gtatcctgag actttgctga agttgcttat |
| 74281 | cagcttaagg agattttggg ctgagacgat ggggttttct agataaacaa tcatgtcgtc |
| 74341 | tgcaaacagg gacaatttga cttcctcttt tcctaattga atacctttta tttccttctc |
| 74401 | ctgcctgatt gccctggcca gaacttccaa cactatgttg aataggagcg gtgagagagg |
| 74461 | gcatccctgt cttgtgccag ttttcaaagg gaatgcttcc agtttttgcc cattcagtat |
| 74521 | gatattggct gtgggtttgt catagatagc tcttattatt ttgaaatacg tcccatcaat |
| 74581 | acctaattta ttgagagttt ttagcatgaa gggttgttga attttgtcaa aggctttttc |
| 74641 | tgcatctatt gagataatca tgtggttttt gtctttggct ctgtttatat gctggattac |
| 74701 | atttattgat ttgcgtatat tgaaccagcc ttgcatccca gggatgaagc ccacttgatc |
| 74761 | atggtggata agctttttga tgtgctgctg gattcggttt gccagtattt tattgaggat |
| 74821 | ttttgcatca atgttcatca aggatattgg tctaaaattc tcttttttgg ttgtgtctct |
| 74881 | gcccggcttt ggtatcagaa tgatgctggc ctcataaaat gagttaggga ggattccctc |
| 74941 | tttttctatt gattggaata gtttcagaag gaatggtacc agttcctcct tgtacctctg |
| 75001 | gtagaattcg gctgtgaatc catctggtcc tggactcttt ttggttggta aactattgat |
| 75061 | tattgccaca atttcagagc ctgttattgg tctattcaga gattcaactt cttcctggtt |
| 75121 | tagtcttggg agagtgtatg tgtcgaggaa tgtatccatt tcttctagat tttctagttt |
| 75181 | atttgcgtag aggtgtttgt agtattctct gatggtagtt tgtatttctg tgggatcggt |
| 75241 | ggtgatatcc cctttatcat tttttattgt gtctatttga ttcttctctc tttttttctt |
| 75301 | tattagtctt gctagcggtc tatcaatttt gttgatcctt tcgaaaaacc agctcctgga |
| 75361 | ttcattgatt ttttgaaggg ttttttgtgt ctctatttcc ttcagttctg ctctgatttt |
| 75421 | agttatttct tgccttctgc tagcttttga atgtgtttgc tcttgctttt ctagttcttt |
| 75481 | taattgtgat gttagggtgt caattttgga tctttcctgc tttctcttgt aggcatttag |
| 75541 | tgctataaat ttccctctac acactgcttt gaatgcgtcc cagagattct ggtatgtggt |
| 75601 | gtctttgttc tcgttggttt caaagaacat ctttatttct gccttcattt cgttatgtac |
| 75661 | ccagtagtca ttcaggagca ggttgttcag tttccatgta gttgagcggc tttgagtgag |
| 75721 | attcttaatc ctgagttcta gtttgattgc actgtggtct gagagatagt ttgttataat |
| 75781 | ttctgttctt ttacatttgc tgaggagagc tttacttcca actatgtggt caattttgga |
| 75841 | attccctgct ttattattct aaagcaagct tgtccaacct gcggcctgtg gcccaacaca |
| 75901 | aatttgtaaa ctttcttaaa acgttatgag attttttttg cgattttttt tttttttttt |
| 75961 | tttagctcac cagctatcgt tagtgttaat gtattttttg ttttgttttg ttttgagacg |
| 76021 | gagtcttgct ttgttgccag gctggaatgc agtggtgcag tctcggctca ctgccacctc |
| 76081 | tgcctcccag gttcaagcaa ttcccctgcc tcagactccc gagtagctgg gactgcaggc |
| 76141 | gtgcgccacc atgcccagct aactttttgt atttttgtag agatggggtt ttaccatgtt |
| 76201 | ggccaggatg gtcttgatct cctgacctcg tgatccaccc tccttggtct cccaaagtgc |
| 76261 | tgggattaca ggcgtgagcc acctcgcctg gccagtgtta atgtatttta tgtgtggccc |
| 76321 | aagacaattc ttcttcttcc agcgtggccc aggaaagcca aaagattgga cacccctgtt |
| 76381 | ccaaagcatg taattttatt cacagaaaag actctcggcc gggcgcggtg gctcacgcct |
| 76441 | gtaatcccag cactttggga ggccgaggca ggcggatcac gaggtcagga gatgtagacc |
| 76501 | atcctggcta acacggtgaa accccgtctc tactaaaaat acaaaaaatt agccaggcgt |
| 76561 | ggtggtgggt gcctgtagtc ccagctactc gggaggctga gacaggagaa tggcgtggac |
| 76621 | cccggaggtg gagcttgcag cgagccgaga tcacaccact gcactccagt ctgggcaaca |
| 76681 | gagcgatatc cgtctcaaaa aaaaaaaaaa aaaaaaaaag actctcaagg atttaccatc |
| 76741 | taaaatcatc aagtgtatat catgcagatg aacaacagaa aacactggga gtattcaagt |
| 76801 | aattaaaaat aacctttcag caccaacagc aatttctgtc ctgatggcaa tctactcaga |
| 76861 | gctagaggac tgcacttagg ataccaaagg gagcttagga tgcaagagca gcctgcacta |
| 76921 | atgtactctg cgttagttta catccgggca ctttgcttta cgtttcaaaa cagacgccgc |
| 76981 | ccttagctca ttaaagggga aatggaggta taatgtgttt taaagggatg tttcttctct |
| 77041 | gaagccatat ttcagggtag catgaaaaca gagctttgga tatctggttc cattctacat |
| 77101 | gacacctcat gcttgtttca aatgacacca caaccaaggg gcaggaatga aaggaatatg |
| 77161 | ctaaaaaaaa aaaatgcaat cctttttagc aagaaagatc attaaggatt tcctgataag |
| 77221 | tttcttcttt gttcatgtgc cctcctctgc tccccctttg aactactgcc cctgtcaccc |
| 77281 | ccccttcccc gcctccccgc cgtttctcag ttctccaata gggatccagt ctgccagagg |
| 77341 | tctatttttg ctcattattg tcaacacaat ttgccatcaa gagttttcac ttactcctgc |
| 77401 | tatcctatca tcacattatg catgtcagaa agcattacag atttttactt ttcaagaacc |
| 77461 | cgaactcaga tttccaggct tctgtccttc ctgtttcatg ctgttgactc tcccacatct |
| 77521 | ttgtcttcct cctagacctt tggtcactct tgagcccaca ttgccaatgg tctatatcat |
| 77581 | tttcacctgg atgcctccca gacacctcag tcacatcaag ttgaaataga actaattaga |
| 77641 | cagaagtagc atgtgtttca gtggaactga catttttcta gggtccctaa aacacaggtg |
| 77701 | ttgtttcaaa ctcccccctc caacatccct tatattctct ctgtcaccca ggctccttat |
| 77761 | atcatctctc aacatgcttc ctgggtgact actgactctc cacttctggt gacaccccac |
| 77821 | agagcaagct ttcaattcct gtcatttaaa cactttagtg cactcataga atctcatcct |
| 77881 | tctgagattt ctcttccacc agtctatttt gcagaccacc aggctacgcc cttccaaaag |
| 77941 | ctcctttgag atggagtttc actcttgttg cccaggctgg agtgcaatgg catgatctcg |
| 78001 | gctcattgca actgccacct ctcaggttca aatggttctc gtgcctcagc ctccccagta |
| 78061 | gctggggtta aagttgcctg ccaccatacc cagctaaatt ttgtattagg ggttttacca |
| 78121 | tgttggccag gctggtctcg aactcctgac ctcaggtgat ctgccaccta ggcctcccaa |
| 78181 | agtgctggga ttacaggtgt gagaaaccac gcctagtccc ccaaagctcc atttttatcc |
| 78241 | tgtcttcatt ctctcttcaa gaacttgccc acagctgcca gagttccttc taagagttta |
| 78301 | agactctctg ttcatgtggt aagaatcttc ttaaggagct tgctttaaag gtaaaaccag |
| 78361 | atttacacat cagaatgggg agaggtgggt gggagctaag atggaatctg taattttcac |
| 78421 | aagtgccctg gatgattcta ctgcagatgg tctcctattc aactaaatag cactcatctg |
| 78481 | acccactcct tttagacccc acttctgtta agccagtgtt atcgctattt tccactttct |
| 78541 | ttcaagttct aatcatcctt aaaactccaa cagcttcagc tgaaatcagt accacactct |
| 78601 | catccttgtt tcccacccaa aaactattgt actaattgac cagacatgta ttgatgccct |
| 78661 | actatgagcc tggctccatg ataggcactt gggatagaga aatgtaccat aatcctgacc |
| 78721 | ttaataaatt tagagtctaa tggaagagat aaaaaaaatc aatgattcca atgagtgtga |
| 78781 | taaggtaagg ctagtcagaa aagtcaggtg cagaggaggg gcaactaatt aaaccagggg |
| 78841 | ttgggggaga cactgccaac tctgtgttga gtgaagttta agcagaggtt tttctttaag |
| 78901 | taaaaaatca gtttttttaa ataaaaagtt ttttaaaaag taagggggaa tagaagaagg |
| 78961 | aggtttgtcc ctaaggggaa cagtgtggag gaggaaagga ggtgaaagtg tatgctattc |
| 79021 | tgtgtactgg caaattcttt ggtgggtgat atggtttggc tctgtgtccc cacccaaatc |
| 79081 | tcaccttgaa ttgtaataat ccccacatgt catgggaggg acccagtgga aagtaattga |
| 79141 | atcatgaggg caggtttttc ctgtgctgtt ctcatgatag tgaataagtc tcaggagatc |
| 79201 | tgatgatttt ataaagggcg gttcccttgc acatgctctc ttgcctgcca ccatgtaaga |
| 79261 | catgcttttg ctcctccttt gccttccacc atgattttga ggcctcccca gctatgtgga |
| 79321 | actgtgagtc cattaaacct ctttccttta taaattaccc agtctcagct atgtctttat |
| 79381 | taggagcatg agaatagact aatacagtaa attggtactg gtagagtggg gtgctgcagt |
| 79441 | atctgaaaat gtggaagcaa ctttggaact gggtaacagg cagaggttga aacagtttgg |
| 79501 | agggctcaga agacaggaag atgtggtaaa gtttggaact tcctagagac ttgttgaatg |
| 79561 | gctttgacca aaatgctgat agtgatatgg gcaataaagt ccaggctgag gtggtctcag |
| 79621 | atagagatga ggaacttgtt gggaattgga gcaaaggaga ctcttgctat gttttagcaa |
| 79681 | agagcctggc agcattttgc ccctgcccta gagatctgtg gaaatttgaa cttgagagag |
| 79741 | atgattcagg gcacctggca gaagacactt ctaagcagca aagcattcaa gatgtcactt |
| 79801 | gagtgctgtt aaaagcattc agttttatgt attcacaaag atatggttcg gaattggaac |
| 79861 | ttatgctcaa aagggaagaa gagcataaaa gttcagaaaa ttggcagcct gatgatgtga |
| 79921 | tagaaaagaa aaacccattt tctgaggaga aattcaagcc tgctgcagac atttgcataa |
| 79981 | gtaagatgga gccaaatgtt aatcaccaag acaaagggga aaatgtctgc agggcatgtc |
| 80041 | aaggaccttt gtggcaaccc ctcccatcac aggctcaggc ctaggaggaa aaagtggttt |
| 80101 | tgtgggccca gcccggggac ccctgctcta tgcagtctag ggacttgttg ccctgcatgc |
| 80161 | cagttgctcc agccatggct gaaaggggcc aaggtacagc tcaggccatt ggttcagaga |
| 80221 | tgcaagcctc aagccttggt ggcttacacg tagtgttggg cctgtgggtt cacagaagtc |
| 80281 | aagaattgag gtggatgtac agaaatgcct ggatgtccag gcagaagttt gctgcagggg |
| 80341 | tggggccctc atggagaatc tctgctaggg gagtatggaa gggaaatgtg gggctggagt |
| 80401 | ccccacacag agtgcccact gggacactgc ttggtggagc tgtgagaaga gggacaccat |
| 80461 | cctccagact ccagaatggt ggatccacca acagtttgca ccatgtgctt ggaaaagctg |
| 80521 | cagataccca acaccagccc atcaaagcaa ccacaagggg ggctgtaccc tgcaaagcca |
| 80581 | caggggcaga gttgcccaag gctgtggtgg gaacccacct cttgaatctc tatgagacat |
| 80641 | ggagtcaaag gagattattt tggaacttta aggtttgact gctttattgg atttcagact |
| 80701 | cgcataaggc tgatagcccc tttgttttgg acaatttctc ccattttgaa caggtatttt |
| 80761 | tacccaatgt ctacacccac attttatctg ggaaatagct aacttgcttt tgattttaca |
| 80821 | ggcacatagg tggaagggac ttgccttgtt tcagatgaga ctttgaactg tggacttttg |
| 80881 | agttaatgct gaaataagtc tttgggggac tgttgggaag gcatgatttg tcttgaaatg |
| 80941 | tgaggacatg agatttggga ggggccgggg tgaaatgata tggtttgtct ctgtgtcccc |
| 81001 | agcttcatca ggatcctccc acacatcccc attctaagtt gcagggtccc attcttttcc |
| 81061 | aatcaatgcc ctcattttaa cagtagacac ctggtgaggc tgtgcatgca cctttcattg |
| 81121 | caggtcagcc gctcccatga taagagcttg tatctgattt tccacaattt cagctctttc |
| 81181 | tctacaagag ataagactct cactctggac aatcttagaa gatttgaggc tcagtgtatg |
| 81241 | cttctggagc tgggagttag aatccctaag ttcatggttt tcttctatca gtttgtccag |
| 81301 | tgaacttagg agcaaccaac caacttcgtt atattccttg gtcctctata tatggtcaaa |
| 81361 | ggtattatgt atagagtcat taaactcctt gcctctcatg agtggtgaat caggagtact |
| 81421 | gaatgcattt cttttgcata agtgtttgaa cagttcgtgc caaggactat cagtgttctc |
| 81481 | cacacaatta gaagtagagt ccttagcatt ttggggtcta atcatattaa gcaaccaact |
| 81541 | ccagaaaccc aaaaaccaac taaagaaatc tatccttctg taatcccagc actttgggag |
| 81601 | gccaaggcag gcggatcacg aggtcaggag actgagacca cagtgaaacc ccatctctgc |
| 81661 | taaaaataca aaaaattagc caggcatggt ggcgggcacc tgtagtccca gctactcggg |
| 81721 | aggctgaggc aggagaatgg catgaatcca ggaggcggag cttgcagtga gctgagattg |
| 81781 | cgccactgca ctccagcccg ggtgacagag tgagactcca gctcaaaaac aaaaaaaaag |
| 81841 | aaaaaaaaga aatccatcct taaaattctg ttcctctaga accattccca gtaccaaaat |
| 81901 | ctgattaaaa aaaaaaaaca ggcagaggaa ggtggaagga ctagatcttt ctccagtgct |
| 81961 | ggatgcttcc tgccctggaa catcagactc caagttcttc agcttttgga ctcttggact |
| 82021 | tacaacagta atttgccagg ggctctttgg cacttggcca cagactgcag gctgcactat |
| 82081 | cagcttccct atttttgagg ttttgggact cagactggct tcctgactcc tcagcttgca |
| 82141 | gatgcctatt gtgggacttg gtatcttgtg attgtgtgag tcaactctcc taataaactc |
| 82201 | cccttcatat attcatctat cctattagtt ctgtaccttt agagaacact gactaatgcc |
| 82261 | gtggggttaa gccaatcgtc tagtaggttc ataaaatgct aaaagcagat ataaacttaa |
| 82321 | agacgatcca gctcatctct tcattttaca gatgtaaaaa cagaagtaga aatagaattg |
| 82381 | gaaccccagt ttcttgaaac ccagtgtttg cataatacca tgctaatttc attctaattt |
| 82441 | gtgttttatt taataatcag gaaacagttc aaatagaggc ccagggctct gaaacattgc |
| 82501 | ccaaggtcta tggcttttgc aaagcaagta ctgtttctgc tactttacct agttgctctt |
| 82561 | ggcctatgtt tggggtgcat ctaaagaact gtttgctgat tattaaataa aacacaaatt |
| 82621 | agaatgaaat tagcatggtg ttataaaaac acagggtttc aagaaactgg ggttccagtt |
| 82681 | ctatttctac ctctaggata cagggtaagt cacttccttt ctcagattct gtttctttgt |
| 82741 | atgtcaaagg gctggattag ataaacttga cttcccctct tggccctcat gtatcattct |
| 82801 | ataaatatgt tatcatttct aatagactgt ttgatgtaat cttttgtcta atggcccctg |
| 82861 | cttttcacaa taaatgaaac aaaggtcaca agacttttat tcatttgcaa ccctgattaa |
| 82921 | ctaacagtta atgtgtaact ggagtgccac atagaaacag aaaggagaag gggaatggat |
| 82981 | tgggttggag aaggtgaagt ctgacctatc ctctacaaga ggtacagggt ttatccagga |
| 83041 | agacaggagt gccatgagga gtaaactcca gcaggggctg gagtcatggc cacacacagg |
| 83101 | gccttacagg atccaggctg cccagagcag agtttggatg ggggctggga ggctggggga |
| 83161 | gctagctgag gagatggttg tcatgccagg ccacgagtgt ggatgagtgc tggccagggg |
| 83221 | aggtatagga gattgatggg gctgtggcaa gcagggcaga tacccttcca ggaacctgtt |
| 83281 | tcattaaaca cagaatacag tcctgagggc tcggtctcaa tacagtcctg agggctcggt |
| 83341 | ctcaggaaaa catgttctta agttttacat ccttcttctg ttttgattag gtgtttcctg |
| 83401 | attataaaat aaattcctag aactgttaat ggtaacaaca caaagcacta tagacccata |
| 83461 | cgaggatcat attgaaacca atgacattta agaataaaag atctgatgaa tatgaacact |
| 83521 | tccctggttc ctaggaggat aacagttgag ttttgctcag gtaatctgcc ttctcttcct |
| 83581 | tcttctctgc tttRtatctc agtttctata ttttgtctta cattaatagg atttgttctc |
| 83641 | attaccctga tgattttatg gtaaactacc tcaaattctt tttggaagaa gatgggatat |
| 83701 | aaattatttt taaagtttat ctgaatagat gttcttcaat atcacacaat aaataatgac |
| 83781 | aatgtaactt tgtgcatata gcactttaag agattgatca taaacacaat cctattaatc |
| 83821 | ttaattcaag ttaataatgc ttgcatttgt atggcatttt aggtattata aagttttttt |
| 83881 | taattgtggt aagatatgca taaaatttac tatttaaacc tttttgaagt gtatagttca |
| 83941 | gtagtgttaa gtacattcac aatgttgtgt aaccatcacc actagccatt tccagacttt |
| 84001 | ttcatcatcc caaactgaat ctctgtacct attaaacatg acctcattct ccacctcccc |
| 84061 | acagctcctg ggaacctcta ttctactttc tgtatgaatt ttcctattct aggtgtctca |
| 84121 | tataagtgga atcatacaat attcatcctt tggtgtctgg cttatttcac gtagcgtaat |
| 84181 | gctttcaagg ttcattcatg ttgtagcatt tatcagaatt tgattcattt ttaaggctga |
| 84241 | atatcttcca ttttatgtgt ctaccacatt ttgcttatcc attcttctgt tgatgaacac |
| 84301 | ctgggttgtt ttcacctttt ggctattgtg aataatgctg ctatgaacac tgatgtgcaa |
| 84361 | gtacctgtct gagtctctgc tttcaatttt gggcatatac ctagaagtgg gattgctgga |
| 84421 | tcataggatc attctatttt taactttttt gaggaattgc cataccaccc gctacagcag |
| 84481 | ccgcatcttt tatattgcca acagtgcaca aaggctctga tttctccact ttctggtcaa |
| 84541 | catttagatt atcattcttt ttttaaaaaa acgtaataSc taacccaatg ggcatgaagt |
| 84601 | aaggttgttt ttgttttttg tttttaatgt gtgcgttatc ttacttgatc cattaaatcc |
| 84661 | ctaacaagaa ctcctctagg gcagatgtcc tgtcttattc atccttggcc ccagtgtctt |
| 84721 | gcaagcaagt gaattctcaa taagtgttaa ttgaatggat ggttgataga tttataggat |
| 84781 | gcatgccaat tctgtggacg agagtaggta ctaattgtta ttttcatttc acagaagagc |
| 84841 | aagtcagggc tccgagcact aagtgacttg gctgaggtca aactgcctgc aagttttatc |
| 84901 | taatagtgac agaggaacca atgtgtcgag catgaatgtc agtccattga aacagtgccc |
| 84961 | acttttctga ctctgctcct taagagacag ggcctgtaca gcaaggacac agagaagcag |
| 85021 | gttacagaaa aagggctggc tcatccgtgt atgcctggca tttgaggaat gtggctgaaa |
| 85081 | tctcaacact ctggttcaga agcacatctg caatcaaata taacaagaca tggtatgaga |
| 85141 | gatgtctggc ataccaagga gattcctaga atacagcgga taggaaaacc tcattacttc |
| 85201 | aattcccaag aaagagtact actggtagta atcccaacag gagtatccaa gtaactcggt |
| 85261 | aatcttcagt aaagaaaaga aattgtgaaa caattatagt cagtcctcca tctctgtagg |
| 85321 | ttccacatct gtggattcaa ctcacctcat attgaaaata ttaataaata aataaataat |
| 85381 | aacaatataa caattaaaat aatgcaaatt ttaaaagcat aacaactatt tacatagtat |
| 85441 | ttatattgta ttaggtatta taagtaatcc agagattatt taaaacatat gggagagtgt |
| 85501 | gcataggtta tatgcaaata ctacaccatt ttacataaga gacttgggca tctgtggact |
| 85561 | ttgttatctg caagggtcct ggaaccaatt ccccatggat actgagggtc aactgtacca |
| 85621 | agYtgaaaac aaaacaaaag gaaatctatt gcaaaaagga aattcccaaa gataaaaagc |
| 85681 | atgtctatga aatagtcaaa gagccgaaaa tgttgggaag acccacactg ctttccctgc |
| 85741 | cctgtgcccc tttttgcaga tctatttgtg tctttatcta ccagcagata ttctattatt |
| 85801 | ctaatagatt cctgtttttc ccaagagggt ttacttattt attaaatatg atgcaaacat |
| 85861 | ctctctcaga gatgttgctc tctcttccta ggattcctta gccctaaatc tgcagagcca |
| 85921 | attaggtatt aatattgggc tttacaactt tggaccatct gaccaccaag actgattcat |
| 85981 | taaatgtata tggtttcaga tcacattaat ttatcataga ctttagaatt ggctttttag |
| 86041 | aagtactgct ataaggaaaa tctcagcata gcaagtttta tctaatagtc tacttgtatt |
| 86101 | aaagagtact caatgtaaac cctaggaaga cttttaactg ccttttggaa attggttgag |
| 86161 | tgggatttga accgtgtact ctctgtaaag caggaaatta tcacttagta attactaagt |
| 86221 | atttaaaaat gggaaataga aaattacatt tcagacctgg tgcagtggct catacctgta |
| 86281 | atcctaacac tttggcaggt ggaggcaggt ggatcgcttg agcccaggag ttcaagacca |
| 86341 | gcctgggcaa catagtgagc aactccattt ctactaaaaa taaaaaaaat tatttgggcg |
| 86401 | tgatggggtg cacctgtagt ccccactact caggaggctg agacaggaag attgcttgag |
| 86461 | cctagaaggt caagcctgag tgagcagtga ccggccattg tattccagcc tgagcaacac |
| 86521 | agcgagaccc tgtctcaaaa aaaatgtata tttttaaaaa aagaaaatta cattttagtt |
| 86581 | gttccataaa tatcagtaca accaaaccta agtgcaaaat tcccaacaca aatgatcgcc |
| 86641 | tctgggccag ccatgtagcc cccatcttgc tgtcaataat catttccagg ggctgtaatt |
| 86701 | gtcttccttc cccctgtacc accccatcac aggacacaaa ttgttttgct tagtttagat |
| 86761 | agctgtgact aaactaagtg ccaattgtct tttaaaatat gtgttaatca gcactcaaga |
| 86821 | ttgttcctac aaaaatgtca ctccctcact caatttgcat gtgccctcct gagtaggaga |
| 86881 | gaaagagctg agttagaggc agccctctgg gacctgcaca gaaccctgta tgcttccggg |
| 86941 | agtgtggagt gtgtgtctga tctgctgctg ggaaaaggag aagaaatgct gagacactca |
| 87001 | ctgccagggg ctggtatcag gccattttca caggggctgt tggagggttg acagcacagc |
| 87061 | tctactggac cagggagggt cccagccagc agggcctgcc cctccgaaac tgtccctgtc |
| 87121 | cctgtccctg agaggcccca ctgagtgtca gatggcacat aagagatttc cttatgcgtt |
| 87181 | ggtgtgaagg agatgatcag ttccaggagg cccctccccc atgcagaaga gaagaaaatg |
| 87241 | gaagaaaccg ggttctcaga gtggcctgcg ggtgagtgcc gccttgtgct gtcagttccc |
| 87301 | ttcaccttcc agttctgggt gtacctagtg tggtttcatc aaactgctga ggccctggaa |
| 87361 | ttgagggagg atgctgaagg gtgccaggcc atagaagcag tagcaggagc tgcagcaaag |
| 87421 | agctagtgtg tctccagcgt cagcttttgg gccttggtgg catcaaagga gacgtacaca |
| 87481 | gggccactgt actacggatg ggattcttgg aacaaaagtt tgaataattg atgagtgtgg |
| 87541 | aagtgctatg caaaaattca attcaacctg cagttaccag gcattcattc tgtgacaggc |
| 87601 | attattgtga gggtggcaag gagacgggga gtctctgaga gactcagaga agagcagggc |
| 87661 | atggtgtcca cccacaagag atctgaagac tttcaaagta gttgggaaaa cagacataca |
| 87721 | acttttttcc cctccccaca gctagttgag gcaaaagaca tataacttat acagtgacaa |
| 87781 | aatactactg actgttactg acacataaca gcctgggctg ggtcactggt gagaaaaagg |
| 87841 | aggattcttg cttttggggc attcaagtcc tgtccattaa ccatttccca atcccatatt |
| 87901 | tatacatccc attttagaca tattaagctg aagattgatg tgacataccc aaagacactt |
| 87961 | aaggttgaag ctgagaatct gggctagaaa tataaatcag gatgggctgg gagcggtggt |
| 88021 | tcacgcctgt agtcccagca ctttgggagg ccgaggcggg cggatcactt gaagtcagga |
| 88081 | gttcaagacc tgcctggcca acatggtgaa accccatctc caccaaaaat ataaaaaatt |
| 88141 | agccaggtgt ggtggtgcat gactgtaatc ccagctactc gggaggctga ggcaggagag |
| 88201 | tcacttgaac ccgggaggca gaggttacag tgaaaggaga tcgtgccact gcactccagc |
| 88261 | ctgggtgaca gagcgatact tcgtttcaaa aaaagaaaaa aaaaaaaaag aaagaaatgt |
| 88321 | aagtcaagat taaagacaat gggtgagatc agcaaggagc atgtgtgcgg agaagagaac |
| 88381 | accaaggaag gctgcgtgtg gtgggaggtg gccggggggc agagaaagag gcggcggagc |
| 88441 | caaggagata gggcatcgtc tgaatggtga tgctgtatca acagatgtga aattcccaga |
| 88501 | ggtgtgaaac acagcagatc ctttacagca cgatgacagg acacagcatg agcctacctc |
| 88561 | tgccaggtga gaggagcttt ctgcaacctg tgatgggctc aaggatgctg accattcatc |
| 88621 | cgcccattgg gatagcccag gctctgagct cagcactcca cccgtcactg cgattgcact |
| 88681 | aatcctcacc cacccttgca ggcaggtatt actgtgggca cagagaagtt tgctaacttg |
| 88741 | ccagagatcc tgtgtaggaa gccaggtcag gaaacaacca gagtctctct aacagcccag |
| 88801 | gccttgaatg aacaccggcc tgcttcagaa tacatggccc gtgatgtgtt tgaattcaca |
| 88861 | gattcactgg acaggttccc tatagggcct gtgaggaaat ctggtctaac agaatccaga |
| 88921 | aagacaaatt tcgctaaaca ggtcaagcct gatactgctg ctacacttca gctgtgttaa |
| 88981 | gccactcggt tatcagacct gcttctcctt tcatgatttt agtaacacag gcctcttcct |
| 89041 | tgggcccctg ttgctcccac catctcccag gttctcttgt actcagggtt tagtctcccc |
| 89101 | tccccactac taatgatgca tgtgccttac tctctgatca tctctcctaa atctgctact |
| 89161 | cctctgctct ttctgtgcct tatctatccc tgccgagtct aacatgcaga ttttggtcat |
| 89221 | tccaaagtca tgtatagatc taatcacagg gctctctgct taccagctgc tacttggata |
| 89281 | aggaaagcat gccaacacgg tcctccttct tcatgctggc caagtcagca tcattattat |
| 89341 | tacctaagtt tatttctaac acatctcaat atcttcatgc actccctctt gataaaagta |
| 89401 | actgagcata gcaccatcaa taccatcaaa tctgtcattc tcttcccctc tctctgggtg |
| 89461 | ggacaggaag ccaggctgct ctaggaaatc ttccctaaca agcaaagggg acttgcctgt |
| 89521 | cctctcgcat gtgtgatctg agcttgtgtg gatcccagag tgggcattct ggactacttg |
| 89581 | accttgccta tctctccttc acaccctctc atctctccct cctaccacca aaaaacttgc |
| 89641 | atcgtatttc caatctccag acatactttt gaaaccattt atctatctgg tgtgtttatc |
| 89701 | tgtatctagt atgatgtgaa tatgtgattt gtatgtgtgt ctaccactgt tttggtcagt |
| 89761 | ttgtatttct cgtgggtaca gttctatgtg ctcatgtatg tggaacatat gtatactgac |
| 89821 | acatggacct agctccaaat gatctgaaag gaatataatt gtaattgaat atttgcacag |
| 89881 | atatacaaca tacacatgtg atggctgggg gaaatgcatg tgggatttca gtcagcattt |
| 89941 | tattagagaa ggtatgtcat tagtgctgta ttaacaatga atcagcttat ttgtgggtca |
| 90001 | ctgtcaatga ctcctttgca aatcacacat gtaaatattt ctgtctgtgg tctgatgaac |
| 90061 | atgcagtgcc acagtctggg agatgctgag ccatgccctg tgtaggcagc atatgaaaag |
| 90121 | aactgcatga tttaaaagat gctgaccagc ttaaggaaag caatttaaaa acttcctaaa |
| 90181 | aatctagttt gaatgaacaa cagttttcat tttgtgtgtg tgttttttct tttagagatg |
| 90241 | gggtcttgct atgttggcca cgctggtctc taactcctgg gatcaagcaa tcctcctgcg |
| 90301 | gctcagcctc ccaaagtgat gggattacag gtgtgagcca ccgtgtctgc tggccccact |
| 90361 | gttttaaacc ctgattcgac aatcatacat ttaactcatt acctgtcttg ttccttttac |
| 90421 | gacaaactca aagcttttat tttactaaag tatttgggtt aatcttttgc ttttctgtca |
| 90481 | tgttctgaaa tatgtacaat aacagaatcg ctcaaaatat tatctccgtt aaatgttttg |
| 90541 | tggctttagg gagaggtcta acaacatgcg ggaaacaaga aatcaagcgc atccaggatt |
| 90601 | catttataat ctctctcgtt gagtagaagt ccgcatctct cgatattgtc tggttacctg |
| 90661 | catgaagtat tctaaaggag gaaaatagct caaagaggac attcatgtgc actctggctt |
| 90721 | ccagttggcc atttgagtaa gtgatcgcaa ttaactgacg agcggcaggg aaacacttcc |
| 90781 | tggaattctc atctacagac aagaacaaac tggggcgggg cccatcacct tcacctacgc |
| 90841 | gccgggaggg tggcggctgg cgggcggggc cgggctcggg ccgtgacgcc gagagtgcgg |
| 90901 | ggcgcgcggc tgggagcctc gcgcccccgc ccgggcccgc ccccatcccg cccgcataca |
| 90961 | gcccgcatcc cgccggggaa gcgagcccag tccagcgctg cccgtccagt cctcgcccaa |
| 91021 | gatttaaagc ccgcaagttt tgttcttgag accagcgact ttagctccga tgcgggaagg |
| 91081 | aaagccgacc tccgatttgg acatttaaag agctgggctt gaacttcgtg agtttcgctc |
| 91141 | taaactgccc ttgaaatgaa gctggacttg gaggtaaagt cactgggaag ctggcctggg |
| 91201 | gcggggtttc cccctcttct cgtattttag aaacggacag cggcagtgca gccctagttt |
| 91261 | gctgtaagtt tccttacttt gttactgagg cccccagagc tccacgcata agtggtgtga |
| 91321 | ccagaaacct tttaacaaga cccgcctgag cctgcgttag agctcccgct cggaaagtaa |
| 91381 | aagaccatcc taatccgcgg cgctgcggaa ccggtgtccc gtgtgggagg aaccgcggcg |
| 91441 | ttccctgggc gtagggcccg cgaggccagc acagtccgcc tcttggcgga gcgccctggg |
| 91501 | ccggtggttc cgcgcggagt tagtctgtgg tcagttacgt ggtgaaaaca cggctgtgcc |
| 91561 | gcggccgcat ctttccgcgg ccgaggcctc tctgggtggg agtgttggct tcctttccgg |
| 91621 | atcgctaaat ggggaaagtt ctggccgctc ggcgggatac gtctccaggc cacggatggt |
| 91681 | tcgttctccg tgccgcggcc ccgagctggg ctccctgggt ctccagcgcg ggctcccggc |
| 91741 | attgggggct gcgggccggc ccctccgccc cgcccccgcc ccgccgcgcc tcctcggccg |
| 91801 | agcggctcgc ggtctccggc gcgggaggct ccgagtctgc ccactccggg ccgagcgagg |
| 91861 | tctctggagg agaagagtgg cgaggaggtg agggcacgcc ggccctcgcc cggcgggtgg |
| 91921 | cgccaggact tcaggtggga acgcgcgctt gggccggggg cgcgtggctg gcgtggacac |
| 91981 | cggatcgggg cccgccgccc tggcccggac cgcgcacggc ccagcgccgg gaagtcggga |
| 92041 | agccggggag gcctctccca ccgcgggccc cggcagcccg ccctctgaaa gcgcggcgga |
| 92101 | gaaggaggct cgtcccctcc ccggaacgcc tttgttccct ccggcctgcc cgcgcgggtg |
| 92161 | gccagcggct gggacccagg ccgggccgcc gcccaggtgc ggcaggtagg ctcgggggcc |
| 92221 | gggcagctcc ggttggggcg gcttcccggg gcctgcgggt ccccgtccct gaggagctcc |
| 92281 | ggctcctcgg tggcgggaca ggcccgtgcg cgggagccgc gaggcgaacg ccgcgcccac |
| 92341 | caattcggtt gccggccggg ggccccaggc ttgcggccac ccgcctccgg ctggagggct |
| 92401 | gaattcgagt cgaaagcccg tgtcgggctg gaaagaagaa accgccaacc tgagaacgct |
| 92461 | ttcggcgagt tactggcggg ggaaatgggg acagggaagt gggcaggcgg ggagactgca |
| 92521 | gccgcagatc tccctggcgg ggaggtcgtg gccactcttt cctttgactc tgcctcattt |
| 92581 | cattttgaat cctgatgtga cagaggcaat tgcttgcttg gataccatat aggtaaaagt |
| 92641 | aacagttttc aactcgactc ttgactacac cctgtacatt cttggccggt gttggttttc |
| 92701 | ttaggttatg gatcatgtta aaggtacacc gatgtggtaa ccgcacagtg gccgatggtg |
| 92761 | gcctgaggct catatttatg gtaattatct gatgaaagta catccatcag aattggattt |
| 92821 | gtgcgtctgt gcctttattt tgggaaacct tgcctgttcc ctgtggggga tgggagagga |
| 92881 | atgtgaaaag ccgaagttga cccagaaaag gatgattgaa ggtagttgta ttaagtggtg |
| 92941 | gcaccgaaat gattccaccc tgaactttct gaaagggtga ttagtgatca gcagcagacg |
| 93001 | ttataacttt ctcaaaataa atttatggta gattttctat ctggtcacaa gtgaggaggt |
| 93061 | gaaagctgct ttttggggcc aactctttgc ttttaaagca agctaaacgc aataccagaa |
| 93121 | aggtttccat tctgtactta acctgcttgt ccttctcact cttccttatc ctcccgcaca |
| 93181 | cgctctgagc attgactgag cactctgagg aggaggctga ctcaggctga cccttcccgg |
| 93241 | ccctgcaagg ctctaaggta gaaccagtgt tatcacagga aaggcccaag ccaaagctca |
| 93301 | gagcagctgt tcctgagaaa gtgggagatg aggatgagta ggaggtagag atgcttataa |
| 93361 | ctgctctgcc cagagaaaac tcagaaggtg cagaagagtt ttcaaaataa agtgcaggcc |
| 93421 | ccatcaagta aaaaatgaaa cattgtattt cattaaaatg gtgatcagtt ttctcttttc |
| 93481 | ataaaagaca ttcaaatggt gagattgtaa aaaaaaaaaa aaagaaaaag aaaactttct |
| 93541 | aattctcaaa aatagaacag cttgaataat aactcatggg ttttgaaatg ggtcatcttt |
| 93601 | taaaatgggt cactttggct agaatcattg cacttggtga ctttcacatt gtaaagggtc |
| 93661 | cagttctttc tgaggcccaa ttgcactaga atctgcattt cagaacacag gaggtatcag |
| 93721 | gaagaaactg ggagaaattc agagggaaat tgtctcttct cagagtaaca aaatgtcccc |
| 93781 | tattcagggg gaattttata tatgctgaag taaatagatc tcagctttga atttttataa |
| 93841 | tacattatgc cttttctgaa atatttccat agccattatt ttaatgcatt gttagaataa |
| 93901 | ccttgtaagg agaggaattc attgtatctg tgttccagaa actgaggcac agtaaattac |
| 93961 | aagagctctg ccctttgata tcgaatctct gtgagcgaag atgtaaccag aacagtgtaa |
| 94021 | atctagaatt actttcctta cggtatgcaa tatctaatta ttatgtatta gtttacatat |
| 94081 | atatagggga tgtaagaatc tttatattta aaggataaga aacttgatca ggtgagatac |
| 94141 | aagatttgaa taaaaaagcg attttttaga aacattttaa ttctacaaat tcagttgctg |
| 94201 | gctttgatat gtaacttatt cagatttctc ttccatagaa ggctttaaca ctagaagcca |
| 94261 | gttctctaca aaagagaatg ttctaccaag tgtgtaggca acaaagccca aaagttcaag |
| 94321 | ttcaaaatac tgttctacag cctaagatcc acctaatatt tcagatttga ctcttttgct |
| 94381 | cctttcctat aacttcctat aactttttca tagtgctcta agcaaagtaa gttcaaggat |
| 94441 | ggactcacct caagtttcct tctcttactt tagaggacca tataatctta tacctgtgtc |
| 94501 | atcctaattg aaatgtattt taagtgcttg tgggaacaag aataacggaa gaccgttatt |
| 94561 | ccattaatgg aataatgggg aacatgagaa ggaccatatt cagtcaatta gggatgatac |
| 94621 | ttctgcctca acagtatttg cgatgtgtta aatggccctt agccatcaga tcagtatttt |
| 94681 | taaaaactcc tacctaatta atgttttttg agaagaagca tattatgagt atagctcagt |
| 94741 | attctaaaaa gaaaaaaacc tgaaaaaaaa atcagccatc cattaactaa cccatcctgg |
| 94801 | taaagctaca caaacagatt ctagagaaag gaaatttgca cagatggaac atctaatctg |
| 94861 | gactgacatt gtcaacaagt tatctcaaat gatcctgaga aaacactggt acatggttta |
| 94921 | gagacaagtc gagctcatgt gactagtaaa tggagaggca cagtatacat ctacttttgt |
| 94981 | tggcatttaa acactctcgg cttttttact ctctttcacc accatcaggt ccagattcca |
| 95041 | gcttgcactg gaatatttat tgaccctttg atagaagagc tacacctgaa acatctctga |
| 95101 | agtcttttta gcatcctatc ctgcccatgg cctactacac agtatgtgat aagttaatgt |
| 95161 | ttgttgaatt agttaaatat acgtattaac ctactttcag ggctgcccgc tgtaatgcct |
| 95221 | agaatagggc aggcacttaa taaatggtag ttaacttaga ctaaatgttg atccaccaag |
| 95281 | acagaaacat gttacatata cctcttgttt tacaccatct tctacttttt gtacctctgt |
| 95341 | gttataaacc ctctatcaag tgttgcttct aaagacaatg tccagaatgg gacgtaggaa |
| 95401 | ctgccactgg gcaacaggca gacactgggt tatatcgtca tcctgtttct gaatcctggt |
| 95461 | ttttcttatt attattcaac cacagctaaa ctttctgatc tccttttttc cgttttttcc |
| 95521 | ctttacccta acttctagcc aaactaagtg atttgacatt tccccactca agccacaact |
| 95581 | ttttctctta tgtgcttaaa aaaaattcct gcgtagtctc tcttggaatg tcttattcct |
| 95641 | tgctccctcc acttcaccct tttttttttt tttttttttt ttttttgaga cggagtctcg |
| 95701 | ctctgttgcc caggctggag tgcaatggtg ccatctcggc tcaccgcaac ctccgcctcc |
| 95761 | tgggttcaag cgattctcct gcttcagcct cccgagtagc tgggattaca ggcatgctcc |
| 95821 | accacaccca gctaattttt gtgtttttag tagagatggg gtttcaccat gttagccagg |
| 95881 | atggtcttga tctcccgacc tcctgatccg cccgcctcgg cctcccaaag tgctgggatt |
| 95941 | acaggcatga gccaccgcac ccggcccact tcacccttta agccagttga aatgctcctt |
| 96001 | gccctactcc tcccttctcc ttttctttct cccatccaag tactaaccag gcccaaccct |
| 96061 | gcttagcttc tgagatcagc caggatcagg tgcattcagc ggggtatggc tgtagacttc |
| 96121 | tctttctgag ctgtgtcttc ctcctttcct ccccaaaccc acatttgaat tctgttctag |
| 96181 | ctcctgctca tttgccacct ctcccacaaa gccttcccca gtttccatct tctccctgct |
| 96241 | tccctcatct cagaggaaag ctcttcattc acagagcacc tggtttgggg tccatagttt |
| 96301 | gttgtccctc agtatccttg gggaattagt cccaggacac cacccccccg cccctgatat |
| 96361 | ccatgcatgc tcaagtccct tatataaaat ggtacagcat ttatattata acctatgcaa |
| 96421 | tattcccatg tactcttttt ttttattttt atgtgtttag agatgggtct cactctgttg |
| 96481 | tccaggctag agtgcagagg catgtagcct tgagctcctg agtcaaatga tcctcctgcc |
| 96541 | tccgcctccc aagtagttgg gattacaagt atgagctacc acacctggcc catgtacttt |
| 96601 | aaatcatctc tagattactt ataataacta atgcaatata aatgctatgt aaacaattgt |
| 96661 | tacacggtat tgtttagtgc ataatgacaa gaaaaaaatg tgcatgttca gtacagacac |
| 96721 | gaccataaat ttttttttca aatgtttttg atctgtagtt ggttgaatct gaggatgcgg |
| 96781 | aacccatgaa cgtacacgac caactgcata tgggtttcta ataacagatt tgtggatgaa |
| 96841 | gcccacccaa cataatgcaa catttcaaat gtatcttagt cagtttttct ggggcaaagc |
| 96901 | cagcctaaca aatctcaaga atgtttgaaa aattctagct tgagtgaacg ttgttcttct |
| 96961 | agtgactcaa ataaagcagt cattgtgtct gaggagtcct agtggggtgg caggggtggg |
| 97021 | gctaggagta gtggctgttt gtagcatgtt ttatatcaca ttaaagggct gggtatgcat |
| 97081 | tttaggcaat aattcttact cttattgata ggagaacttg tattttttat tcatctacct |
| 97141 | tatgatggat aactggttta catctcttca tcaggtttac ttcttacgtg tcattttatg |
| 97201 | agctgacttt gaatatatct tcagattttc cctgccttta cacatgaaaa cggttttgat |
| 97261 | acagcttttt cattagagaa tggcagcttt tccaggccag ggtgtctgtg tctgactcct |
| 97321 | cgcctttcat tttacccaat acttgcttaa aacatcctcc ctgagtccgg tacttttttg |
| 97381 | cctcctctcc ttccttccac cccacctctt tttgtcaccc tcctgcgact ttggatctcc |
| 97441 | atccttggag tccctgaatc ttttttgtgg ttgaaagact acacccaaag gacacagact |
| 97501 | gatgaggtca cttctgccca ctgtctagta ctagtgagac caagaagaag aaggagggaa |
| 97561 | aactgaaatg gaaggtttga aaaagagtgt gttagtgaag gaagtaagaa cagtgctaac |
| 97621 | agaatgcagc aaaacagaaa tgacttcaca cttctggcag tgaagcaaat tctcatccat |
| 97681 | cttgggttgc ttctcttgac cctgcccacc tcctcccctg ccctcccctc ccgatgagaa |
| 97741 | ctgcaggaac acctgtgccc atgccaagga aaaagggcag gtgaggaaca cagggatggg |
| 97801 | gctgggactc acaagctgct ctaagctgcc acagggtctg gaatttgttg acattacatg |
| 97861 | gttaactcgg tatctcgatg cctcagtatc ttctgcaaaa tgtggaggga gatcctttct |
| 97921 | tcctcaaagg ttgttgtaag gcttaaagag ctaccacatt cgtaaggcat ttggaacagg |
| 97981 | gcctggtata tagtagacat acccatggta gtaactgttg ctagactggg taaaaggaga |
| 98041 | agtttcttaa gtaggcaaga aaaaaactga caacagttat atttaaaaga gcaagcaacc |
| 98101 | gtgtcagccc aaacaagtgt gttgcaagaa tgagcacaga gggccaagct cagaaaagcg |
| 98161 | agtgtttgac ctctctgaga cctcctttat atgtcagctt ttttatttgt ttcaaatttt |
| 98221 | gcataatcaa actggttact gggaattatt gagttcatat aacttcccta aaatctaaga |
| 98281 | ataatctata atcccatgcc accagtcccg ttaataaaga cttagggtct gtctttaccc |
| 98341 | atttgttcat ttgaaattcc ccgtctttac ttcccccata accaggaaac agatctacac |
| 98401 | ccatgtttaa cttataaaga gatataagtg agtttacata ggtggagatc ttgtacctgg |
| 98461 | attcacattg taggttttat tacccagctt tcccctctat cccaaagcca tgattgccat |
| 98521 | agtggaattt aaagtctttg gactaacact gaatcaagaa ctaactctac aaatgtattc |
| 98581 | tctgcaggag gataagaatt ccaaatggct tctaattgtt gcccatggct tgaaataaag |
| 98641 | ttccatatag ctaaagcccc agcacaaaaa cacttgaaga cagccattag gtagaatttc |
| 98701 | ttttcttttt tagaggtctt atattggaaa tgtaacagtt gcaaagatat ctaatgtttc |
| 98761 | accttaaaat gtgagttata aattcacatt tcacattgcc tccttcctcc ttgagcaaac |
| 98821 | agactaggca attagtacca aatagtaact agttatattt cttaacaccg attattgtaa |
| 98881 | gtaatttatt aagaaaaaat ctaggaagat caaattatag cttttccagt aaatctgggg |
| 98941 | ctttttattc ctgatctctc tcaacaaggc agtttggctt ctgaagattg gtcctagcta |
| 99001 | tacttaacag gaaacagatg acgagaagaa gccaggaaaa aatacttgga atatagaaca |
| 99061 | aaatgcagca taaagatggt acagaagatt ttatctttct ttcctttagt tatgtgtgga |
| 99121 | caggaactaa aaactctccc acgtgggact ttattaagta ataaaagttt aaatgtaaaa |
| 99181 | gatataaagg aatgaggata taaaggaaat ttttttaaaa actgtgactc accggaggag |
| 99241 | ccttatatag tctactttct aagcatatca cttttaaagg gatcattata ttttcatgat |
| 99301 | gcatggggat attccaagta ttaaattgat tttttaaatg aagaaaatta tcagttcagt |
| 99361 | cttgttttct atgtaggttt cactacagta ttttatttcc cccgcaagga aaaaaccaga |
| 99421 | gctagaatgg aatgattaaa tttcttagtt ttcttctaat ctgttagtct ttccattaaa |
| 99481 | ttatactgcc ttcttatata gaatttctgg gtttgttctt gttttggcaa taattgaagt |
| 99541 | aagagaggat gattttatgg tagaggtttg attccattct agattggtgt ttttcagcta |
| 99601 | tgtagaacac aatccattca tggttcagac aaccgtttta ggaggttttc atgtccagca |
| 99661 | ttaaaaaaaa aaaaaaaaga atagaattgg gcattcatat atatatatat atatgaatat |
| 99721 | ttattaataa aagtttaaaa gatataaagg aatgaggaat gcatatatat tatatatata |
| 99781 | tataccaaat taaagtatca taatactaat acaaggatta gtatcgtttt atgatacttt |
| 99841 | attatataca ctaagggtta gtcttgtttt atgatacttt attatataca ctaagggtta |
| 99901 | gtcttgtttt atgatacttt attatataca ctaagggtta gtcttgtttt atgatacttt |
| 99961 | aggttggtat acatacatat actaagaatg cagacatgtc catggaagtt ggcaatattc |
| 100021 | agcccccagc aggccccagt cagcaggaca gggattaggg gagtcaggtg aggcagggct |
| 100081 | gtataagtgc agggttggag cctgtcttgg tttaccgttt tgacattttg ttcactgtgg |
| 100141 | atttttttca gtaactttga ttttttaaaa atattgcatc aaaatattat actgactacg |
| 100201 | gagtttttgg taccccctta aattgtgcac ctaaaataag tggctccctt gtctccccct |
| 100261 | agtcccggcc cgatcagtat gttttataga gggaatggtg agtgttgggg aactagggag |
| 100321 | cccatgagga ggcggcccag aaggggagga ggagctgatg gtgtgtctga tgtctcctcc |
| 100381 | tttctctagg aagcagacag gaggtgaagt cttcagggtg ggggggacag agcctgggag |
| 100441 | ggggagattt agcatggccg ctgaagagac tgggaaaggg agaaggttcg ggacaagttc |
| 100501 | aggaacagtc acgtagcact gaaaactgct caaagctaga tagtgccatt tgtggtgacg |
| 100561 | ctgcctgcct ggtaagtccc atttccccag cagccctgga ggtgtcatag gaacaggaag |
| 100621 | atgggagggc gctggggtct ggggttgttg gggtgggagg gcaaaggtgt ccaatgattc |
| 100681 | tgcatctgaa acgggggagg tgtgtgagga agaaaggtca ggagactgct gggcagggtg |
| 100741 | gggtggcaga aggttctcga tgaatattta gaagttgcag ctgaggagtg agtgtactat |
| 100801 | agagtgagta tcctggaatt tgaggtctca gaagaaatgc agttctcagt gaagccatgt |
| 100861 | aaacaacttc ctggctgata aggtgtcttg agtgttggga gatcacatga tcaaaatggt |
| 100921 | atacacacca taaggtgtcc ttagacttga gtgtggtgta agtctactga ataaaatgca |
| 100981 | gaaatacagt tcactgttgc ttttcttcct ggaaattgcc ctccccaacc tcaaaccagc |
| 101041 | ccccaagtgg ggcagggtcc atctttagac cctcctgtag cacccagcac aaccctaact |
| 101101 | atatggtcaa tgtctctctt ccccacttag agctccaaga ccagggcctt gtttgtcctg |
| 101161 | cttacccact ctacccattt gttaacccca tcttaatttt ctgatgaatg agcccttttt |
| 101221 | tcattgactc ctcctttctc tccttctccg gagtttattt ttcctccagg cccattgctg |
| 101281 | tctctccacc tagagagctt atccacattc atgaaattag tcatcttcat gctcagagta |
| 101341 | cccacctgga tctctccagt cctgtctaac tcgtcttaga ttattacccc ctggacatct |
| 101401 | gtaccttctt gtctctccat cgcctcgaac tcaatgcgag tctaaaatca aatactcctt |
| 101461 | cactcctaac tcaggaaacc agttttccct cccttgttgg taatgcttga aattagctct |
| 101521 | tacttctctt cttcctcctt tactcgccat cccaggtcat tcatcaggtc ctgctaggtc |
| 101581 | tttgattttc ttgaaagggc cttctttatg ccttccttcc gtccccattt ccaccaccct |
| 101641 | cgactaggcc ttcctcacct catccggcat cagtgtgagc ccttgagaaa ccagcgtcct |
| 101701 | gatcttcagg cattctgcat ttcaccccat accaccatta gccagtttaa tccctcttgc |
| 101761 | tcaaacttaa taaatactga gcaagggagt tactataaca aacaaaacca gaaatttcta |
| 101821 | tgacttcctg ctgctttctg taccccccta agactcctct acctgatttt caaggttctc |
| 101881 | cataagcagg ttcccacctc tctgctttca aatcttctat ctgccataca cagttcttgt |
| 101941 | ctaaaacaaa gattgttcct actcctgcag ccacctaaat cctagtcgtc ctcaggccct |
| 102001 | cctaaaatcc tgactaatcc aacccccacc gatttctcta ctagtcccag agacttccct |
| 102061 | cttgtgcctg gggttggtgt catgcagaca cactgtacct taccaggatt tgcccattgt |
| 102121 | tgtctgtggg aactatgctt tcatgtggga actacgagtc ttgtcacaaa tagagtgtaa |
| 102181 | atgctttgag attggagact gtaaaatact tcttcatgat ctcccactgt gtctggtata |
| 102241 | agaacccgca gagtaaatac tcatatactt gtaggattaa ttgaaacaac tggagactga |
| 102301 | ggttgtttaa cttgtactta gaagtgtgga cctccccatc ttgattaaaa cttcaaaacg |
| 102361 | gatagcaggt ttcacatatt atctcagcat cacaaaagta gactcaacaa agaaatgagt |
| 102421 | tgacagcagg ggtaaggttt acaacaatca gggattattg cagggaaact tgtagccttt |
| 102481 | gggtgttcta actagttttt tcccaaaaag tttaaagagc tctcttgtga aagaatattc |
| 102541 | ttatgagtaa ctgaggggca cggctcctag actgaaaagt atttgggatc tgtcacctct |
| 102601 | tttctgatgt tcctacttct attctttatt cttctgacta cctctattag aaaagataat |
| 102661 | actaagaata cctgtccctt cttctcagtc caaatagaag caaacccagg ttgtatctga |
| 102721 | gatatctgca tattttcttc taagcaattc tttgtcctct tctctcccat gctttttcta |
| 102781 | tttcctattt tcagcaggct ctgaagtcat ttataatttt tactgcccct cggtgacatt |
| 102841 | acatggatat tcatccctga tttgcagatt aaagcaccga atcagagtga ggtgagggtt |
| 102901 | gcccaaggtt acacaataaa tctggcccca aacaggggta acccttgagt ttctagtctg |
| 102961 | ttgattggcc actgacccgt gctgcaggca cacaaaggaa gctgcaccca cagcagtctg |
| 103021 | ttgtggatgg ttgctgagct gcgcattcgg cattgggctt gctttgtttc ctgccaggcc |
| 103081 | cagcattttc ttctaccaga tcggcaggct tgtgggcttc ttcctaggtc cctcccctgc |
| 103141 | actctgaata ggaaagctgg aagctgtgct ttagagaagc tttaagacgc cgaaagaaac |
| 103201 | cagaagagtg agcgccagtt gtatgtgcgt ggtctccatc cgcaaagccg gagctgggcg |
| 103261 | caacagtgtt gacttgtaat tgatcaattt agatcgggcg caggccgggg gagggcagtg |
| 103321 | cttttgattt aggctgggaa aggcctccta gtgactatgt tcaatttgga ggaattcaga |
| 103381 | tgttcttttg ttatacaagt gaagctgtgt aatacaaatg aggagtttta cttttcctaa |
| 103441 | atcttcccct tatcattcaa gtattgagga gttttacctt tcctaaatct tccccttatc |
| 103501 | attccagtat tatcagtgag atctggttgt gatttatgta aatggtggct aaaaaattca |
| 103561 | aactactgag ggggagaatt ctcattttac agcttcacat gctgtgctga actaaataag |
| 103621 | tagcgtggga tgttggcttt gtgacaggtc ttttgtcatt tttcagaaag cattttgact |
| 103681 | tgttgatgtc aatttggaac agctgaaaaa atacaggaaa ataagataaa tacgtacatg |
| 103741 | ttgagggtgg ggacaaaatg aaggttctga accagctgcc ggcttacagt agccatataa |
| 103801 | gcaacagcag caatgcacca acctggtgag taataggcct gattcactgg agagatacta |
| 103861 | gcacctttaa tgagtcagat agatgcacaa tgggtgtggg agcagttgga cttgtgggca |
| 103921 | caaagtctag caagaagctc agacttgcaa acaactgtag gacgtgcaaa gcaagctggc |
| 103981 | attggagctt gccgggcaca gctgctcagg aataggcagc tggttttccc tttgatccct |
| 104041 | gagattccaa aggttacttt cctctttgtt cccttcccag ggtcaattag agtagaaact |
| 104101 | gcagatgctt ttcagttgag aattttccta gaattctcaa aaatgtgtat gctggcttaa |
| 104161 | aatctgccat caagaattct gttaccttgc tttaagcctc cagttccttc cagatgtatg |
| 104221 | gtggaggagg ccagagggcc cttgttttgg ggcttcagag gatggttgtt atctggatga |
| 104281 | gcactgtgga aagactgaga gagcaactga gagaaagtgg gcccctgaat gaaagtgatt |
| 104341 | tcgcaaattt taggcagatg ccaccatcag aaactgatat tttctgacgt ctttctcacc |
| 104401 | ttcctctaga gcattcagtc cagaaatgac cagcctgtcc aaagggggaa attactgata |
| 104461 | ttgatctgtt ccttagagca gtgtttcagt cttttttttt tttttgagat ggaatctcat |
| 104521 | tctgtcaccc aggctggagt gcagtggcac gatctcggct cattgcaacc tccaccttcc |
| 104581 | tgattcaagt gattctcctg cctcagcctc ccaagaagct ggaattacag gtgtgcacca |
| 104641 | ccacacccgg ctaatttttg aattttttat agagatgggg tttcaccatg ttgccaggct |
| 104701 | ggtctcaaac tcctgacctc aagtgatcct cctgcctcgg cctcccaaag cgctaggatt |
| 104761 | acaggcgtga gccaccatgg ccggccttca gcctttgtga tattaaagca cagcaacaca |
| 104821 | tttcccatta cacccctgaa cacacacaca cagaaaaccc aaaagtttca caaaatgatt |
| 104881 | cttgctctta ctactctcag tacactctgt atttaaaaaa aaaaatgctg gttgtggctt |
| 104941 | cctaagtggt gcgtgcagtt ttcaaatcaa tgcccttggc gataaagtgt gccctatact |
| 105001 | gattatctct ggacaaagtc tgaatggggc ttggctctaa tctctagtcc tcattggaca |
| 105061 | ttttacatac ctggcctttg cctccaccct gatgtggagt gatcatgggg gtgggaaata |
| 105121 | tagctggatc cgaaagctct gaagtgggga tggaggtgtc acagctgagg ctaggcccat |
| 105181 | tctgcagggc actcagtgtg tacagttggt tttctatcag gggtcaaccg gcggggggac |
| 105241 | ttgagaacag atctctgggc acaaagcagg gcctttgccc tggggcttgc tatgtggctc |
| 105301 | agcctacacg gctctctccc cgtcagtcct gtccaaagcc caggaaacta atgtaccacc |
| 105361 | cccgaggaag agagcctacc tttccatcca aggaagtgtt ttacctgtgg taagcacggg |
| 105421 | ggacagaatt cttgaggaag gagggtgctg cgtcccagtg gtggaggaaa agagaggacc |
| 105481 | tggtgtaagc agccatggca tggacctcat ccgaggtggc acctggctag ggtcctgacc |
| 105541 | tccaatcctt ccccagtaac catcactttg agtaaacagt ggctccaccc ccggcatggt |
| 105601 | tctttgcacc aacatttggg gaatgcctac caggggtcac acactgagct ggatgctgag |
| 105661 | tgtagggtgt ccacaacatc gtgcctaaaa agtctctgta tggggtataa gaaggtgctg |
| 105721 | gggcaataca gatgagatga gaagcatctt tcagggaatg ggttgatccc aattcaggct |
| 105781 | tcccagagaa ggatgtctgt agacttcata ttagcaaggg aggaaggtag ccaggccaca |
| 105841 | ggactgctgg tgtaaagacc agggcatatg aaatggcaag tgtgactgtg ctttcagcca |
| 105901 | ataatttggt attgtcaaat gatgggacca aacagctgga gaggcagatc ctaaagggtc |
| 105961 | ctgtgggcca ggctggactt catcttgtca ctaactaatg gagaggctct gaaggagtta |
| 106021 | aaagagctca gtttgtctcg tggttaaatc caagttttac aaaggtcacg ctgactgtaa |
| 106081 | agtggaaggt gggctggcca ggggatcatc tagtctgggt gagaagtgat gataacatga |
| 106141 | aggggtgaag agagatttag aagaagtgat tcacaggatt aaacatttaa ataatggaag |
| 106201 | tggagaaaat ggggggggcg gttccagatt tcaggcatag atgaaagaag tgcagttagg |
| 106261 | cacatgtaaa gagaaacagg aacagcaggt tttaggggag aagataacag aatgggtgag |
| 106321 | aaatgacact tgagtaccct agtgtgctag gtaatcatct gtctacttcc cttcatttgt |
| 106381 | catgtatatt cccatttaat ttgcataaag acttcgagtt aaacggtctt accccaattt |
| 106441 | gtcaaatttc tgcgcatgat atggtacaag aaaccgtaag tggctaaggc ggcattggtg |
| 106501 | ttcaaattgc ctgactacaa aggcagtgct tgttggctac attctgttgc ttcccagttt |
| 106561 | agaacatgtt acattgaggc gcctgctgca tttccaaata aaaaagtaca gaaagaaggt |
| 106621 | ggctgtataa atctggggct cacaaagtaa ttttgattac tgagagtttg ctttcaagga |
| 106681 | gcaaactgtg actccttgat tatgaacctt aatttaaaaa aaaagaaaaa agaagtctta |
| 106741 | ctcttattcc tgccttgtct ggggcaagcc ttaatggatt tttactgctg tgaattttct |
| 106801 | tttcattgaa gattttgcct tgatctatgt atctgctttc atcctgacca tattcaagtc |
| 106861 | agtatattca tgaatgtacc tgtttgtgaa atttgaactt aagtatacac gattatagcc |
| 106921 | gtttgggaag cttttttttt ttttttttta agagtaggag tagaaaaagg tctctgtact |
| 106981 | ctgaatggga agacagtgta aagcaatttt ttcccttttc ctgtcctcct ttaaaaaaaa |
| 107041 | taaacagccg tatgcctctg ctaagtacta actacctcat caccttttgt gcagacaggg |
| 107101 | caggttacat ttggttttaa ggaattagga atatgtttct ttccagcacc ttagtaaccc |
| 107161 | acgcgattgt gattcttttc tcttcttgac tgtgataggt ggcatggaat attcacatgg |
| 107221 | gagagccgca tgaggccgcc caccacgctt cctgaaggat gcccgtgtgg aagaattttg |
| 107281 | acgtgccagt gtcctcgttc tacagggtgt tccattcttc cgcaatctca gaaaaatggg |
| 107341 | actaaaagaa actattttgt aaaataagaa gacttccatt tttaatgacc aacatgtatt |
| 107401 | aagatggaca cctactctac gaaacacgaa gttctatggt ctcgaagaag cccgtgcctg |
| 107461 | tttaaaactg atcctaacta aaaacagact tgagtggata tgagaatgtt ggttagtggc |
| 107521 | agaagagtca aaaaatggca gttaattatt cagttatttg ctacttgttt tttagcgagc |
| 107581 | ctcatgtttt tttgggaacc aatcgataat cacattgtga gccatatgaa gtcatattct |
| 107641 | tacagatacc tcataaatag ctatgacttt gtgaatgata ccctgtctct taagcacacc |
| 107701 | tcagcggggc ctcgctacca atacttgatt aaccacaagg aaaagtgtca agctcaagac |
| 107761 | gtcctccttt tactgtttgt aaaaactgct cctgaaaact atgatcgacg ttccggaatt |
| 107821 | agaaggacgt ggggcaatga aaattatgtt cggtctcagc tgaatgccaa catcaaaact |
| 107881 | ctgtttgcct taggaactcc taatccactg gagggagaag aactacaaag aaaactggct |
| 107941 | tgggaagatc aaaggtacaa tgatataatt cagcaagact ttgttgattc tttctacaat |
| 108001 | cttactctga aattacttat gcagttcagt tgggcaaata cctattgtcc acatgccaaa |
| 108061 | tttcttatga ctgctgatga tgacatattt attcacatgc caaatctgat tgagtacctt |
| 108121 | caaagtttag aacaaattgg tgttcaagac ttttggattg gtcgtgttca tcgtggtgcc |
| 108181 | cctcccatta gagataaaag cagcaaatac tacgtgtcct atgaaatgta ccagtggcca |
| 108241 | gcttaccctg actacacagc cggagctgcc tatgtaatct ccggtgatgt agctgccaaa |
| 108301 | gtctatgagg catcacagac actaaattca agtctttaca tagacgatgt gttcatgggc |
| 108361 | ctctgtgcca ataaaatagg gatagtaccg caggaccatg tgtttttttc tggagagggt |
| 108421 | aaaactcctt atcatccctg catctatgaa aaaatgatga catctcatgg acacttagaa |
| 108481 | gatctccagg acctttggaa gaatgctaca gatcctaaag taaaaaccat ttccaaaggt |
| 108541 | ttttttggtc aaatatactg cagattaatg aagataattc tcctttgtaa aattagctat |
| 108601 | gtggacacat acccttgtag ggctgcgttt atctaatagt acttgaatgt tgtatgtttt |
| 108661 | cactgtcact gagtcaaacc tggatgaaaa aaacctttaa atgttcgtct ataccctaag |
| 108721 | taaaatgagg acgaaagaca aatattttga aagcctagtc catcagaatg tttctttgat |
| 108781 | tctagaagct gtttaatatc acttatctac ttcattgcct aagttcattt caaagaattt |
| 108841 | gtatttagaa aaggtttata ttattagtga aaacaaaact aaagggaagt tcaagttctc |
| 108901 | atgtaatgcc acatatatac ttgaggtgta gagatgttat taagaagttt tgatgttaga |
| 108961 | ataattgctt ttggaaaata ccaaatgaac gtacagtaca acatttcaag gaaatgaata |
| 109021 | tattgttaga ccaggtaagc aagtttattt ttgttaaaga gcacttggtg gaggtagtag |
| 109081 | gggcagggaa aggtcagcat aggagagaaa gttcatgaat ctggtaaaac agtctcttgt |
| 109141 | tcttaagagg agatgtagaa aaatgtgtac aatgttatta taaacagaca aatcacgtct |
| 109201 | taccacatcc atgtagctac tggtgttaga gtcattaaaa tacctttttt tgcatctttt |
| 109261 | ttcaaagttt aatgtgaact tttagaaaag tgattaatgt tgccctaata ctttatatgt |
| 109321 | ttttaatgga ttttttttta agtattagaa aatgacacat aacacgggca gctggttgct |
| 109381 | catagggtcc ttctctaggg agaaaccatt gttaattcaa ataagctgat tttaatgacg |
| 109441 | ttttcaactg gtttttaaat attcaatatt ggtctgtgtt taagtttgtt atttgaatgt |
| 109501 | aatttacata gaggaatata ataatggaga gacttcaaat ggaaagacag aacattacaa |
| 109561 | gcctaatgtc tccataattt tataaaatga aatcttagtg tctaaatcct tgtactgatt |
| 109621 | actaaaatta acccactcct ccccaacaag gtcttataaa ccacagcact ttgttccaag |
| 109681 | ttcagagttt taaattgaga gcattaaaca tcaaagttat aatatctaaa acaatttatt |
| 109741 | tttcatcaat aactgtcaga ggtgatcttt attttctaaa tatttcaaac ttgaaaacag |
| 109801 | agtaaaaaag tgatagaaaa gttgccagtt tggggttaaa gcatttttaa agctgcatgt |
| 109861 | tccttgtaat caaagagatg tgtctgagat ctaatagagt aagttacatt tattttacaa |
| 109921 | agcaggataa aaatgtggct ataatacaca ctacctccct tcactacaga aagaactagg |
| 109981 | tggtgtctac tgctagggag attatatgaa ggccaaaata atgacttcag caagagtgac |
| 110041 | tgaactcact ctaaggcctt tgactgcaga ggcacctgtt agggaaaatc agatgtctca |
| 110101 | tataataagg tgatgtcgga aacacgcaaa acaaaacgaa aaaagatttc tcagtataca |
| 110161 | caactgaatg atgatactta caatttttag caggtagctt tttaatgttt acagaaattt |
| 110221 | taattttttt ctattttgaa atttgaggct tgtttacatt gcttagataa tttagaattt |
| 110281 | ttaactaatg tcaaaactac agtgtcaaac attctaggtt gtagttactt tcagagtaga |
| 110341 | tacagggttt tagatcatta cagtttaagt tttctgacca attaaaaaaa catagagaac |
| 110401 | aaaagcatat ttgaccaagc aacaagctta taattaattt ttattagttg attgattaat |
| 110461 | gatgtattgc cttttgccca tatataccct gtgtatctat acttggaagt gtttaaggtt |
| 110521 | gccattggtt gaaaacataa gtgtctctgg ccatcaaagt gatcttgttt acagcagtgc |
| 110581 | ttttgtgaaa caattattta tttgctgaaa gagctcttct gaactgtgtc cttttaattt |
| 110641 | ttgcttagaa tagaatggaa caagtttaaa tttcaaggaa atatgaaggc acttcctttt |
| 110701 | tttctaagaa ggaagttgct agatgattcc ttcatcacac ttacttaaag tactgagaag |
| 110761 | agtatctgta aataaaaggg ttccaacctt ttaaaaaaga aggaaaaaac tttttggtgc |
| 110821 | tccagtgtag ggctatcttt ttaaaaaatg tcaacaaagg gaaaataaac tatcagcttg |
| 110881 | gatggtcact tgaatagaag atggttatac acagtgttat tgttaaaatt tttttacctt |
| 110941 | ttggttggtt tgcatctttt ttccatattg ttaattttat accaaaatgt taaatatttg |
| 111001 | tattacttga attttgctct tgtatggcaa aataattagt gagtttaaaa aaaatctata |
| 111061 | gtttccaata aacaactgaa aaattatcat gagatgtgta tttaaacttt ttcatgaaca |
| 111121 | ttgcttatat aatcattcct tctgtcttaa tgtactacat ggtcttagcc ctgttcctat |
| 111181 | aggattatca tgttctctgc attatagagc cacctaagat gtactttttg ttaaatgact |
| 111241 | catgctggaa tatctggatg gggagatgtt ctttcctaat gtagtcatgt gccacaaaat |
| 111301 | gacgtttcgg ttaacgatgg atcacatata tgatgatagt cccatgaaat tgtaatggaa |
| 111361 | ctgccctata caggtgtacc attttttatc ttttatttca gatttttact gtaccttttg |
| 111421 | tatatttaga tgtgtttaga tacacaaata ctttccattg tcttacaatt gcctgcagta |
| 111481 | ttcagtacag caacatgctg tacaggtttc tagcccagga gcaataggct ctaccatata |
| 111541 | tctaggtgtc tagtaggcta ttccatctag gttctcgtat gtataacctg ggatgtttgc |
| 111601 | acatcgatgt ggtcacctaa agatgcattt attggccagg cgccatggct cacgcctgta |
| 111661 | atcccagcac tttgggaggc cgaggcaagt ggaccacctg aggttaggag tttgagacca |
| 111721 | gcctggccaa catggtgaaa ccccatctct actaaaaata caaaaatcag ccaggtgtgg |
| 111781 | tggcacacac cagtaatccc aactactcgg gaggctgagg caggagaatt gcttgaacct |
| 111841 | gggaaaggga ggttgcaatg acctgagatt gtgccactgc tctccaacct ggacgacaga |
| 111901 | gcgagactgt ctcaaaaaaa aaaaaaatgc atttctcaga acttatcctc attgttaagc |
| 111961 | aatgcatgac tataatctgt tgagagaggg atgaaatcac ctgtagttat agcgctttaa |
| 112021 | gataccattt gaaaaggtta cgttttcctt ttctttgaca cggttagctg tctgaaatac |
| 112081 | agtcaatttt aaccctaatc tcttaatatc aggaatgccc ttacacctac tttggagtgt |
| 112141 | ctggtgcttc gatatagttg catgtaatgt gctctcatct gtttttacct gattcctgct |
| 112201 | cagttcttca catggcatat tgtgtaactc aatctatatt taaaacttgt aagcatccga |
| 112261 | attatttgtt tatggtagaa ctttttactt gcaagtcgtg gtaggagtgt ttgtagttgg |
| 112321 | tactaaaatg tgatgacttg gaagaattaa ttaatgacct atatttgggg actttaattg |
| 112381 | gatgctatag ctgcaatgag aatagaacca gagaactctt gatatgcaag gttattcatt |
| 112441 | ctgtgataat aatgagagga atattcgatg tctctttgag tcagttttcc ttccgatcac |
| 112501 | ttccgcattc tgcagtgaca caatccttaa tcatagcttt cattacaata ttcctttact |
| 112561 | ccagacctca aagactcctc actgcctcca gcatcaaatc taaactcttc tacctggttt |
| 112621 | tcaacgccct acgtaaactt ttcctccttc attccctata gcttggtttt ttttttttta |
| 112681 | tcactgccac tattatctat cacaaatgtc aatcatgacc ataccttgct taagttggtt |
| 112741 | tcccttgcca agagcactgt ttttcctata cctgttgaaa ttttggaaat ccaattctac |
| 112801 | ctcctctctt cccttaagca tctcttcctt cccttctccc caaatttata tttagtcaca |
| 112861 | tatattatcg tactacctac taatcattcc atgtgttttt ttacgagctg taagttctga |
| 112921 | aggcaaccat gccttgtaca gtgtccactt agggttctga ataattaatc atctccccaa |
| 112981 | aatctgaaag ccttctatat accaagcaaa tttgtttagt tatgcagcaa aactcaaatc |
| 113041 | tataaaatca aaaaggaata aggaaataca gattaaacag ttgcagcaaa gactggtgat |
| 113101 | cttaaggtat ttagtcaaag ctggtggtag aacaaaaaca gtagtcttac agattctacc |
| 113161 | tcttgattaa ctcagtggct aattttgcct tttctcaaag ttcttttgca agaacataaa |
| 113221 | gatatttttg tttctttagt tgagtgctgt aactttattc ctttgtgttt ctcataagta |
| 113281 | tgatttggca gtctgccata cgttttttgt tttttttctt cctctttgag acagggtttt |
| 113341 | gctctgtcac ccaggctgca gtgcagctgt gtgatcacag cttagctcac tgcagactta |
| 113401 | gcttcctggg ctcaagcaat cttctcacct cagtctcctg agtaactgag actacaggtg |
| 113461 | caccccacca catcccgcta aatgttttaa tttcttgtgg agatggtgtc ttcactatgt |
| 113521 | tgctcaggct ggtcttcaac tcctgggctc gagcaatcct cctacctcag cctcccaaag |
| 113581 | tgttggaaag tgttgggatt acagacctga gccaccacac ctggcctgtt ataagttaat |
| 113641 | acaataattc atcaactaac ttaaagaaca ctaagactct tacaaaagta ggtatgagtt |
| 113701 | ttagtaaaag tctcaaaaga taaactgtca cttaaggaaa actagagaac atatcattgc |
| 113761 | caaatggtgt ttttcagaga ttataccatt caacgcccac atgctgaatt gggccattca |
| 113821 | ttataactcc aggaacatgg caatcagtaa gagcccacat gtttctttga atacaccgta |
| 113881 | agtgaaagaa tataaagtag tctagttaat attatgttta atcaaggagc acattcctaa |
| 113941 | agatgtttgt tcattcattc tacagacatt tttcagaggc ctgctaagag tcaagcatta |
| 114001 | tgatagacgc catggataaa agcttacaag tcaaccagcg tgggtataaa taatgtgact |
| 114061 | agcactagaa caggtatcat gatggggatt ctgagtataa atattttttt aaaataaatt |
| 114121 | tccccgtgtt atttttggct tttacctccc taatttaggc tttctaaatg gcacagcatt |
| 114181 | tctgaggatg caaacacctt tctacagagc aaaaacagca tttgtataaa tttgtgtctt |
| 114241 | tggggaacca agagacttta aatgtgttta aaccaataat tcagtcaata tcaacattag |
| 114301 | cttacatgta atattctctt gatagcccaa ttttttaaaa cactgtattc ttagaagttt |
| 114361 | ggtttctaag atgtcacttt aagctctttt gcttgttgct tttgtgggat ccacaaattt |
| 114421 | tgttctcagg tacataaatg aaggttagta tagaggataa atattatgat tcttatctgg |
| 114481 | gaaagacagg tgctgaggtg taaaagagag gatcctcgcc acccatgccc cgcaccccct |
| 114541 | cgccccctgc acccacggat gtgcagtctt acctgcgggg ggaaaggtct ccgagcctgg |
| 114601 | cctgctgctc cagctcaggg ttcccccttt catgatggct ttcaaagatt tcttcactct |
| 114661 | gaagtgaaag aaattttggt aagatttgat attgtaggga cctcctaatc tatatttttc |
| 114721 | tctctcccaa tttctgtgtt tgattttggt tttgagtctc tcgataagca aaatatccag |
| 114781 | tttctcatgc cgctttctca ggttttcccc agccactctg gatccattat ggtttgccct |
| 114841 | ttttggcttc ctttaggcac actaaaaact ccttcccaaa agcaggtatc ggccgggcgt |
| 114901 | ggtggcaggc gcctgtaatc ccagctactc tggaggctga ggcaggagaa ttgctcgaac |
| 114961 | ctgggaggcg gaggttgcag tgagccaaga tcacaccatt gcactccagc ctcagcaaca |
| 115021 | gagcgagatg ccatatccaa aaaaaaaaaa aaaagcaggt gtcctttccc cttaatcatg |
| 115081 | aagggctatt catactttac tgccccaccc ctattgattc ataagaggac agtaaagcga |
| 115141 | tcactgcatt caacatctcc tttttttttt tcttgtaaga aatcaaggtc tggaaaagtt |
| 115201 | gcactcgccc tgagaccaga aagtgctgga ggcagagatg aagtaagccc agtgtaggct |
| 115261 | ttcacagatg cgtgataaca agtctaacta aagaagtacc tgggatacta gttttgccaa |
| 115321 | ttcagctcta aaacaatatg gcaatcttat attccaaata tatatatata tatttgtatt |
| 115381 | tatagtagag atggggtttc accatgttgg ctaggctggt cttgaactcc tgacctcaaa |
| 115441 | tgatccactg acctcagcct cctaaagtgg aggaaacata tatatatgtt ttccttttaa |
| 115501 | aataggatgt cagtccaata agaatctaaa ttttagttcc ctctaatata tatatctgac |
| 115561 | tagggaccag ataatatttt tcatgtgtca atatataaaa gttggccagg tgcagtggct |
| 115621 | catgcctgta atcccagcac tttaggaggc tgaggtcagt ggatcatttg aggtcaggag |
| 115681 | ttcaagacca gcctagccaa catggtgaaa ccccatctct actaaaaata caaaaattag |
| 115741 | ccgggcatgg tggcaggcac ctgtaatcca gctatttggg aggctgaggc aggagaatca |
| 115801 | cttgagcctg ggaggcagag gttgcggtga gctgagattg caccactgca ctccagtctg |
| 115861 | ggcgacacag tgagaccctg tctcaaaaga aaaaatatat atatatatat attttttatt |
| 115921 | atattgttta ttaaacaaat aaaaataaaa gatatctcat cagttacgat gtaaatgaaa |
| 115981 | aaattgtgtg tgtgtgtgtg tgtgtgtgtt atgatgtact tcttgtgagt tgtggtcagg |
| 116041 | ctttgaaagc cactatgtat gtgtgtgtgt gtgtacataa aagtaagtat cagtcccagg |
| 116101 | attcaaattc gaaccagtcc cagtcaaatt caatctaatt attttcacca cactaccaaa |
| 116161 | agtctttctt cacattttct atataaagtt gaactaatta atacagcagt gaatgttaca |
| 116221 | ttgtatcctt ttgcagtttc tcatctatga cattactatg cctgaattcc ccactggact |
| 116281 | ttgaactcag tcttactcat ctttggatcc ccagcgtgta atacaggccc ttcataaaga |
| 116341 | gtgaccatta ttactaacaa aatttcctct cactgtgaaa cctgctccca attataaaat |
| 116401 | gaattgtgct ctttcgaact ccgttgatct attgtatgca gggtggatca tagagtctta |
| 116461 | tttcataata acctagtgat ggaaaaataa caatgattca ggagtaggta atggaccttg |
| 116521 | tcatctttta cactgaatga tgagatttcc ctaatttata gattttgtca tgcatgagat |
| 116581 | gccctcagga gcttgcagaa atgccttggc acgttgctct gctgacatgt gtcagatggc |
| 116641 | tggtgtggag gtagagggag tctcctctgc caagcaagtc taatggaata taacactggt |
| 116701 | gccattgagg atgcaatgag aaggacccac agcagatcca gagactgcat tcataaaagc |
| 116761 | tgcacagtat accatgtttt attaaggtat agacagatta gttgtgttct agcatagtgg |
| 116821 | ttttcaaagc ctgaccacaa ctcacaagaa gtacataaca cacacacaca cacacataca |
| 116881 | cacacacatt ttttcattta catcgtaact gatgagatat cttttatttt tatttgctta |
| 116941 | attttttatt tctttttttt ttgagacgaa aggtgaattg tagccaaagt acttttagga |
| 117001 | aattttaaaa ttacacattt tgaaagctct aggaggaaga gagcatattc aagtaaaacc |
| 117061 | tttcttttat tcaaatagta gactaaaaaa ttgaccatag actaactcag caattgctgc |
| 117121 | catatttctt agtgagggat tccttatgag ctccattgaa tgataaaagg atactcttcc |
| 117181 | aaatcagagc aacaatcgtt tcttgtgaca ctgcatcctt tttcccttcc ttcaccctga |
| 117241 | attgccccta gggtatacct aggagaggga gggctggcaa ccagctgctg ttgtgaaagg |
| 117301 | gatgtcattc atggcctgcc ctattgggag gccaatcagg cggaacagcc cctctcccct |
| 117361 | tatggtcatt ttatcctgaa gaaaaagggt ggcaaaaaga tggcaaaaaa gattgctgat |
| 117421 | cacagatcac tgttacaaat acaacaattg gctgggcatg gtggctcacg cctgtaatcc |
| 117481 | cagcacttcg ggaggccgag gcgagtggat cacgaggtca agagttcaag accagcctga |
| 117541 | ccaacatggt gaaaacccgt ctctactaaa aatacaaaaa ttagccgggc atagtggcgc |
| 117601 | atgtctgtag tcccagctac tcaggaggct gaggcaggaa agtcacttga acccggaggt |
| 117661 | ggaggttgag tgaaccaaga ttgtgccact gccctccagc ctgggcgaca gagcaagatt |
| 117721 | ccgtctccaa aaaaaaaaaa ggagggggtg gcaatgagac agggaactgc tctgggtcca |
| 117781 | actcccctca catagcccca acccccatat ccaccccaca aacctccacc tccccacttc |
| 117841 | cagcctctcc agtttcaaag tgacgcttac caacacgcag gctctccagg agcaccttga |
| 117901 | aaggtaactt cttacatctt tccaaaacac ttataatcta attgttcttt ctcccctata |
| 117961 | ttttgaagaa atgcatgctc tcaaaattga aaaaaacgcc tagaggaatt ttgtaaaaaa |
| 118021 | attatatttt tccagtttct ccaaaggaca tataaaccct ttttaaactt agattttgaa |
| 118081 | aagtcccaat agctgatttt caatcgatta ttgaaattgt tttttttttc ttccctaaag |
| 118141 | gggagatacc aacctttgaa caaaattaaa ttaacttttc cccaaagtta aatgatttta |
| 118201 | ctttgtgatt ttaggaatgt gtagaaagtg atttcagttc caactctttt aaagcaaggg |
| 118261 | tatctgggcc aggcgtggtg gctccctcct gtaatcctag cgctttggga ggccaaggtg |
| 118321 | gggcagatca cctgtggtca ggagttcgag atcagcctgg ccaacataga gaaaccccat |
| 118381 | ctctactaaa aacacaaaat tagctgggta tagtggtgcg cgcctgtact cccagctact |
| 118441 | cgggaggctg aggcaggaga atcgcttgaa cccgggagac ggaggttgca atgagctgag |
| 118501 | atcacgccac tgcactccag cctgggcaac agagtgagtc tctgtcaaaa aaaaaaaaaa |
| 118561 | aaacaaacaa gcaaacaaac aaacaaaaac aagggtatct ggtaatttaa ggtgaaagta |
| 118621 | ttattcacta ttataatcaa ttttcttaaa aacaggagct attaggccca attctgagga |
| 118681 | gagaaaaaaa agcagatttc aagggcccgt gccaccttct ttgtttaaag catagagaat |
| 118741 | gataataaaa tgttaaaggt tcagcatttt ctggcattgt aaatttaatt aattaattat |
| 118801 | gtaaattaat tatggaaatg agaaataaag tgaaaaggta tgtcaagtaa gaatcaataa |
| 118861 | ttatcaaaac gttctctacc aaatgggatg caggggagaa gggtgagaag aagggaagga |
| 118921 | aataaaggca aaggcatttg cccacagcct cagtccaggg tgttctacat gacgcagccc |
| 118981 | cagagtaaaa aatactagcc acaggaatga gtggctctgt cttcctctcc agaactgacg |
| 119041 | ggcaaaagag ggggaacctc accttttttt tttttttttt tttggaaact cactgggttt |
| 119101 | tgatatggag aacaggaaga gaatctaccc tagcagcaca ttcacagatg gtttgtctgc |
| 119161 | cctatttgct ccatctccct ggctcttcct agcacttctt ttcctacttt ccattttcat |
| 119221 | tggccgcaaa agccacttaa atattccttt ggaaaactgg gcagtctgtc ccctttttaa |
| 119281 | aaccacaact atttccagga gagttaaagg gctttcggac tgcccatcat tcatgcattg |
| 119341 | tggtgaacta gctcagactg cctctacctt tagtccagat cagctgcctt gggagggcga |
| 119401 | ttcctggagg aaaggatgag gagaggaagg ttttaggaca tcgtatggac tgaagccccg |
| 119461 | ttggagggga agggcaggct tgaggaacaa gcctcagtgt tcctgaggct ttgtggaaat |
| 119521 | gagaaatgaa tagagaaaga taagagactg gatcctaaat gcaattttgc ctatttcaaa |
| 119581 | attttcacag ggtctttttt tttttttttt tttgagacaa ggtcttgctc tgtcacccag |
| 119641 | gctgaagggc agtggtgtga tcatagcttc ctgaagcttg cagcctcgaa ctcctgagct |
| 119701 | caagcgatca tcctgcttca gcctcccaaa gcgctgggat tacaggcatg agccaccacc |
| 119761 | cccagcctca gggtcttttt aatccctaag atcttaaatt tcccagctac tattgttagt |
| 119821 | aatgagttca atttacttac tggttaaatt ggcttttatt attattatta tttattttat |
| 119881 | tttattttat tttatttgag acagagtctt gtgctgtcgc ccaggctgtg gtacagtggt |
| 119941 | gcaatctcag ctcactgcaa gctctgcctc cctggttcat gccattctcc tgcctcagcc |
| 120001 | tcccgagtag ctgggactac aggcacccgc caccacgccc agctaatttt ttgtattttt |
| 120061 | agtagagacg gggtttcacc atgttagcca ggatggtctc gatctcctga cctcatcgtc |
| 120121 | tggactaata ctctaaacgc tattggcaat agtttgttta caggaaaaac atcttcttat |
| 120181 | aaagctgact gcaaatgttt taataaattt gcaaatatgt aattctgttt aaaatatcag |
| 120241 | gaagtgagaa acatgttatg tgataactct ttcccattcc cgaaccaaga aaatgtaaag |
| 120301 | gcagtaagtg tgccaaggac actgaaagga agctgcccgt acatctttga atcttctcag |
| 120361 | gctgtttggt ttcatctgat tttaagctcc atggataaat ttcattgtaa caatttttat |
| 120421 | gtctgtaaat cttaacccat aaataaaatc acaaatcaga aaagtacttt attaggccag |
| 120481 | tctttgtcca agcaaatttg gtcccaaagc tagtttacaa ataattgcca cacagctaca |
| 120541 | aggccagtgg gttgactatc gtagcatcaa tggtgtgggc agggctgcag cctctggggc |
| 120601 | agcattagcc ccaactgaca gagggtatag gtgctcttaa caatctatga gaccccccac |
| 120661 | aacctggact cagctgtcat aagcctttac ttcttatgtt cttcctaagt actttatcgg |
| 120721 | gccaacaaat tcatcccatg aggaatccaa gatggaaacg tcaaaactat ctttggtatc |
| 120781 | atgcttcctc cccctacctt ctctgccttg atcccctcct tcattctaca cattttctct |
| 120841 | gatagtcgtg ttctagccac tagggagaac gtttcagtca atgggtatga tttctgctca |
| 120901 | cctctttttg ccatagctat tagtaagctc tcaccactga tgtctcagct gactgtgaca |
| 120961 | actgccagcg gctggtcagc ctgcctgcct tcatctaacc tgctctttac tgtctaccag |
| 121021 | agtcagcttc gtaatacaca ggtgccctcc tcctcctgtc aacattcctc aaccaaagcc |
| 121081 | tcagtaaact gtctctcatc taccttccca gcattcttga gccattccca ggatagccta |
| 121141 | ggccctttag acctctgaga tgttataccc ttcctcattc tggaatgccc ttcagtgtct |
| 121201 | tacccacctt tcagagttct atttattctt tttttttttt tagatggagt ctcactctgt |
| 121261 | cacccaggct ggagagcagt ggcaccatct tggctcactg caccctctgc ctcccaggtt |
| 121321 | caagtgattc tcctgcctca gcctcctgag tagctgggat tacaggtgcc cgccaccatg |
| 121381 | cccagctaat ttttgtattt ttagtagaga cgaggtttca ccatgttggt cagtctggtc |
| 121441 | ttgaactcct gaactcaggt gatccaccca cctcagcctc ccaaagtgct gggattacag |
| 121501 | gtgtgagcca ccacgaccag ccttatttat tctttagagt tgaattcaaa tgccacctcc |
| 121561 | tttgaaagcc atccttgatg tccttgtgag gaattacttt ctctaccaca ttatatattc |
| 121621 | cttttactgt agaaacgtca ttctgcttta tattataaat tattagaaat atgttgtttc |
| 121681 | ccccatgaaa gtttcaagtt tcctgagttt aggcctgatg atacaatcat tttcatatga |
| 121741 | ctcacagcaa ttgccttagt atatattaaa ttgagctgaa taagccatgt acccagttga |
| 121801 | acacaccaaa tattttagtg atgtcatttc tttattggtg aaagagcaag gccaacgttg |
| 121861 | atcttaatgt tattctctct tttatcttat tcagctccct caacaaaaat acattctcat |
| 121921 | agattaatgt aatcaccacc tcactcctct caactttaga ccctatacta gcctgagaaa |
| 121981 | tccagatcca acttaatgag ttctccttta tcccaagtcc tcatgaccta taaatgtaac |
| 122041 | ctctaggaca atgttacaaa gagtgagatc tttataccac ctgcattctt gtcaccaaac |
| 122101 | gtgtaagtta aaaattcaga ttctgagtgt ggtgactcac gcctgtaatc ccagcacttt |
| 122161 | gggaggctga gccaggtggg tcacctgagg tcaggagttc gagaccagcc tgggcaacat |
| 122221 | ggtgaaaccc tgtctctact agaaatacaa aaattagcca ggcatggtga caggcacctg |
| 122281 | taatcccagc tactctagag gctaaggcag gagaatcact tgaacctggg aggcggaagt |
| 122341 | tgcagtgagc tgagatcact ccatggcact ccagcttggg caacaaagtg agactctgtc |
| 122401 | tccaaaaaaa aaaaaaaatt tagattccag gacccgaccc tacacctccc taattagaat |
| 122461 | ctcagagagt ggggccctgg gaatctgcat aattcataac cttctcagag ctctggtaca |
| 122521 | caagaaacca tgagaatctt tgttctggaa acttcagaga acttggctga gggccacccc |
| 122581 | aggatttggt ggcgtccctg aatctcccat tagctctgac actgactaga cttcaaatat |
| 122641 | cacagaaggc aagcattgaa gtgtgtttat attcaaatag ttttcttgtt gagaatcaga |
| 122701 | aatattaata aaacttttgg gagtcaaagt aatgagaaga caaggaactg aacttgcccc |
| 122761 | ctgatctgtt atcatttggc agtaaggtac atattcaaca ataggataat tctgtataag |
| 122821 | acctgaatct gaccttcttt catctttcac actgacaatt ctgtctctaa acacaggatt |
| 122881 | aaatgaggaa ctaaaattca ccaagatcct gctaggtgtc aggtacagtg ctagctgctt |
| 122941 | ccacacattt ctcatttaat cagccacctg tgaaattgct atgatttatc accatttggc |
| 123001 | agaaaaggac atttagggca caagagatta aataagctgc tggttgggta cattaagtat |
| 123061 | taataaataa tcagatagat aagttaggta gttaagtaag ctgccagatt agtaaacagc |
| 123121 | tagtttcaaa tcccagctcc aacatttact agttgtatga cccttgggga aactcaacct |
| 123181 | ctgagtctca tggcactgct gataaaacgg aaaacatcat cctcacaagg ttctgaggac |
| 123241 | tagggatgga ctcaggtaca cacagcaccc agtacaatgt ctggccttca gccagtgata |
| 123301 | gcaacccttc actcacctct ccattccctt ccagcccctg cattcaaaaa cttttattta |
| 123361 | tttatttttt taatttagga ggctctttgc tcagaatcct gaaaaggctt ttgttacttt |
| 123421 | attttttctt ttttttcatc ttctgggagc acagaatagg cttctgctcc tttaaataac |
| 123481 | tttaacaggc accaaaggaa cactgctagc tctttcttaa ttctgtaggt ccacatttag |
| 123541 | gaaaaagaaa ttgtcagcct ctgacttatt tccagcttac aaaaaggctg tgatgttggc |
| 123601 | agcccgggga aagcagttcc cgtgagtcat gctttcacct ttcagccagt gaagaacagg |
| 123661 | aaatagtcac atcactattg ccaacaagca cagcgaatcg cttccaccac cgggctttcc |
| 123721 | cagatgacgt cagtgagcca agtgcagggc atcacccttg ccagatggcc ccggaagagt |
| 123781 | ctcagctgcc ctgtcaagtt tagcttctca agctccccag aaccagcatg gcaaggatca |
| 123841 | cccctccaga aaaggaaatg atttctctga tcatgatcaa accatgtcat aattttagct |
| 123901 | gtaggtggtg agtatcagtg actgtattat agatggtttt ggttcacagc tatttttttt |
| 123961 | ctcggtatat ttactctcaa gggcaaaggg gtggcattta tcacaatgct atgattcact |
| 124021 | ctactagact ggctgggttt catttcatct ttgagttcta gacctaaggc caaaaggatg |
| 124081 | tcagaatcgc cacccagagc tagatgcatg ctcaatgcaa cctggccatc tctctcggac |
| 124141 | agtggccact aaacacaagc tgcaatgatt attgttgatg ttagtcaatt gatttgtccc |
| 124201 | ccttttaata atccatgatc cacacagaca tgaatctaaa cctttttttt gaacctatct |
| 124261 | ttttcttact agttttctta catattttgg aataacaatt tccctggtta taatacacaa |
| 124321 | tgtgtgaaac actaattgca tttattggtc ctaaattcat tcccaagtct aggatttggg |
| 124381 | gatttagtgc acaaatccac tttcttcctg ctcatgttgt tcatgacttt atggattctg |
| 124441 | cttacatttc ctcccaaagc cttggtcttt ctaagcagaa atctgaccag cctctgttcc |
| 124501 | tactctgctt cctccaccac cttgactgtt aacatacact gttgtgggag aagcttttca |
| 124561 | ttaaatacgc aaacaaatca acaataagga agaaaacaac tgaagcacag aaggatgatt |
| 124621 | gtaacatgga cttgtgcttg tttaacagta cccatactgt ttggtgagtt gcctaaatac |
| 124681 | agagggagat ggcaacttaa caggatgggt ttggagtctg aattaaataa acaacatggc |
| 124741 | aaaagaagga agaaaccaaa agttgggtga ctagggcttc ttggattcta ctcaacactt |
| 124801 | tccacaccta caggcctctg attctatccc caccgctgct cctacactgt ctatgtttct |
| 124861 | cttttacaac aacaacaaaa aaatagcact tgtttactgt tcacaattat tgaaataaat |
| 124921 | cactctaatt tggattcctt tatatgagaa cttccatatg cttaaaactc ccgtagcatt |
| 124981 | tttatagtgt acaggtcata aacatgttgt ctgaagatgg gcaaagtagg tcgtagagat |
| 125041 | aggaagtgag ttcacatcac ggcgtgagtc aaggcagcag catatgagct caggggccaa |
| 125101 | atctgagctg tgatcatgtg acacagtcct caccttcaga gatgacattc tcccagaagt |
| 125161 | ccagggtgca gaaatggata ggctgatgtt cacttttgaa gaggaccaca atgaaaccct |
| 125221 | tttaatatta ctcaatgatt ccacagacct cctgggtgtt aaatcaggtg aatctatttt |
| 125281 | taaaaattgc tatttacttt tttaggtggc tgatcaggac agtatgaaat ttcatacagt |
| 125341 | ttcctaactt gagaaaacat ggtgatgcaa ttcctccaac caggagggat ttatggacag |
| 125401 | cggtggctac gtcctaatct gcccagaata caggatgact aaacaaattg gcaaacggac |
| 125461 | gcagctttct ctctctctaa gaaaagtctg ctgagaaccc tcgttcccac tctgtttctg |
| 125521 | cctccaagaa gaaaagccta aaactcactt tcttccggac tctttcaagg tcagtggagt |
| 125581 | tcctctgggg ttcatattca gatactttgg ggattgatga tggatcataa atgttgctga |
| 125641 | agttcatttg agcccatggc ctctggtttc agaaccattc agccagtcaa atatttaaat |
| 125701 | tttagtgagg caggggaaag ggaacccctt cgggggctgc tatacagcaa cttatattca |
| 125761 | caattcacaa ttaaatacac ttcagtttct aaatatatgt tactttaaaa ttatacagtg |
| 125821 | cttagcaatt ttcaaatttt cattttgtgc tcaaaatatt ttcaggaggt agctgttgtt |
| 125881 | atacccattt gtacaagcaa gaaactgagg tttcaagagg ttatgttgct taaacccaga |
| 125941 | tctgcctgat tccaaacctt atgctttttt taaacttcta ttttgaaata attatattaa |
| 126001 | gtcacatgag gttgcaaaga aatggaaagt ctcatgtccc ctttctccaa acctcatcca |
| 126061 | atgttaacat ctaagaaatt gacactggca caatccacag agcctattca gggttcacca |
| 126121 | gttattcacg cacttgtgtg tgtgcatgtg tgtgtgcagc tctgtgcaat tttgtcatgc |
| 126181 | tctttttttt tttttttttt gagacatagt ttcgctctgt tgcccaggct ggagtacagt |
| 126241 | ggcacagtct ctgctcactg taacctccaa cttccagttt caagcaattt tcctgcctca |
| 126301 | gcctcctgag tagctgggat tacaggcatc tgccaccacg cccatctaat ttttgtattt |
| 126361 | ttagtagaga caaggtttca ccatgctagc cagactggtc tcaaactcct gacctcgtga |
| 126421 | tcttcccacc tcggcctccc aaagtgctgg gattacaggc gtgaaccacc gcgcccggcc |
| 126481 | tgtcatgctc tttttataca tgaaccatac actgttctgc caattttaaa tatgaaggca |
| 126541 | gattacagaa ataaataaaa tgatttttct tttactacaa cagtatctac caataatcac |
| 126601 | atacatgacc aaatagcttt cacttctagc tgccgttaag aagaaagaaa aggatgaaaa |
| 126661 | gaaaaaaaat cctataaacc ccagttcttg aaagcattag tctgtgctat gtgccttagg |
| 126721 | tacagattaa ggaaacacaa tttgttgatt tattgataat tgtgacagca atcttccctc |
| 126781 | ttgtcaggaa gttctataag taaaataaag gtaattttac cttgacttca Ratttagtct |
| 126841 | catcatcttc ctttccccgg gagttcaact ctgtctccgt ataaggttcc tcagacgttc |
| 126901 | tcagaggacg agctagaggg actaagagaa cctgccatta gtgtggttat ttaacttata |
| 126961 | aactataact cagtgcactt tgtctgattt ggacaaatag ctggaccacg tcaatgtggc |
| 127021 | taactataaa agctcacttg agctgctgcg ttgatttcct agggctggat gaaattggag |
| 127081 | gtgaaggaga gacacagagg agaaatagaa tccttgactc cagggacttg acatttcagc |
| 127141 | agaagagaaa aaagtcacac ccagcaccat atgaaacatt aaactacaac aaatctgtac |
| 127201 | atatagcaat gtggtaaaga atagtagatt ttctgtcatt cccattttta aggcccagct |
| 127261 | aaaaggtcac ttttggctgg gtttggtggc tcacacctgt aatcccagca ctttgggagg |
| 127321 | ctgaggcggg cagatcacct gaggtcagga gttcgagacc agcccggcca acatggtgaa |
| 127381 | atcccgtctc tattaaaaat acaaaaatta tccgggaaca gtggcaagtg cctgtaatcc |
| 127441 | tagttactcg ggaggctgag gcaggagaat cacttgaacc tgggaggcgg aggttgcggt |
| 127501 | gagctgagat tgcgccactg cactccagcc tgggcaacag agctagactc catctcaaac |
| 127561 | aaacaaacag acaaacaaac aaaaaggcca ctttcctcat gaagtcttcc atattacaaa |
| 127621 | ctttatggat tcttttatgc actgctgctt tggcatgcat catgtgtaca gcataatgtt |
| 127681 | gcacttgatg ttgttcttgg attcaaagac taaattctag gtactacatt gtattgaata |
| 127741 | tattttccat tataaaggta attaattttt gcttattaca gaaaatctga aaaacactaa |
| 127801 | actcttaacg ctgaagcaca atcacttagt atatttcctt acagtcttct ttcaaatgct |
| 127861 | ctccctcttt ttacacacat acatgtacac acacaatcat actatgattg tattgtaaga |
| 127921 | ggatggccat gcctgctgct cttgctcttt tctcttggcc actctatctc ccttctcccc |
| 127981 | acctgggaaa tatcctttct tcaagctcca tggccatttg gtagtaaaat tgggacttga |
| 128041 | gaggagaagg cctcaaggct tcctaaccca ccttaccagc tttgccaagc attgtgggtg |
| 128101 | atggccacaa ggctaataga taagaggtac tttgaattct tatttgtcac agtcaccacc |
| 128161 | cttgcatatg ctggtccctt gggaaactca caggagacat gattaatccc aggcaggatt |
| 128221 | tgagcatttc tttctttctt tctttttttt tttgagacag agtctcgctc tgtcgccagg |
| 128281 | ctggagtgca gtggtgtgat ctcagctcac tgcaacctcc gcctcccagg ttcaagcgat |
| 128341 | tcttctgcct cagcctccca agtagctggg actacaggcg cgcaccacca tacgcagcta |
| 128401 | atttttgtat ttttagtagc gacggagttt taccacgttg gacaggatgg tcttactctc |
| 128461 | ttgacctcat gatctgcctg cctcggcctc ccaaagtgct gggattacag gcgtgagcca |
| 128521 | ctgtgcccag ccaattttag catttgtagg tcccaagacc aaaatgccat tcattgaatg |
| 128581 | ccaccaatat atcaccctga ttcttagcta tggtagactt gatggaggtt gaccatctga |
| 128641 | tctccagagg ccccttccaa cccttccatt ctgtgagcca actggaagca gcttgggctt |
| 128701 | tctatgggtt tttcatagat gttatgttga aaatcgcaga aaattacact gctaccccag |
| 128761 | gttatacata ttaccattaa tgctgctttg taataacaga caatcctttg ggctcctccc |
| 128821 | tctctgtggg atctctataa agtgagtgat tccaggcaca ataactagat gctagaaatg |
| 128881 | catgcactat aatttgtgac accatgactg agaagcgcct gtccttgaca ctgtaagtag |
| 128941 | agtgaagcag tgaaaggcgg ggccacagaa gccacactgc cagtgttcaa accttgctct |
| 129001 | tccatgcacg agacaaacat gattacagtc acctctgtac cttacctttt tcatctgtaa |
| 129061 | agtagggaaa aatgagaaat cctacagtaa agggctgtga taaggcttta atgagttcat |
| 129121 | ttgtgaagaa tgtagatcat atacaatgca taacacattt tagctattat tgccacatgg |
| 129181 | cactaaaaag ttttttcacg ttccttatga acttcagagg tgttagaagt tgtagccaag |
| 129241 | gcatccagct aacaagagtc agagctgaaa ttccagctct taagcccctc cacacatctt |
| 129301 | ctttatactg tgaaatacaa ttgcttatct tggcacagac tcagttacta aggaatcaga |
| 129361 | caaaaatgtt tgaaaatcct tttagtgact tgctggagtc cacactcagt agatacttac |
| 129421 | caaaaacaaa agtgcaggat tgttctgcaa aacgagctag ttgtttgact taggtcaatg |
| 129481 | acaacctgtt cagacttgac aaagacctat ttattctggg aaggagtgtt atctattatg |
| 129541 | aaatcttgta tttgaaaagc tgagaaacaa ccctgtaatt cctgctttat ttacttgaaa |
| 129601 | caaacatatg tacctggaaa caacgaatga ggccaagtca tagatgtgaa catagtttag |
| 129661 | cttgaggaca gacgaaaggt caagagaacc aagttctgtt tccaatcatc taagaaggat |
| 129721 | gtctctgtgt atgtttgggt atgtctgtgg tatgtgtgtg caagtgtgtg agagtgtatg |
| 129781 | tgtgtggatg tgtgtgtgtg tgtttgcaga taaaaattca ggaaagaaat acactaaatg |
| 129841 | ttatcaaggc ttacttctaa aaagtaggat acatggtgga agacaaggga tcgtaaattg |
| 129901 | tactaaatgg agaattacca aaacacccca caattttcat ataaacaaaa actgaaagaa |
| 129961 | tttattgcta acaggccagg catggtggct cacaactgta attccagctc ttagggaggc |
| 130021 | agaggcagga ggatagcttg agcccaggag ttcgaaacct gcctggacaa tatagcaaga |
| 130081 | ccccgttctc cacaaaaagg aaaaaataaa gacaagaaaa gaatttgttg ctagtagact |
| 130141 | ccccaacaat aaatactaac agaagctctt tattccgaag agaaatgaca caagatagta |
| 130201 | attcaaatct ataataagga atgaagaacc ccagaaatgg tgaataaggg ggtatgtaca |
| 130261 | cacacacaca cacacacaca cacacacaca tgcacacaca cacgtatgta tttctcttaa |
| 130321 | tttgatacat gactatttaa agcaaaaatt ataataccat agtgggggat taacatatat |
| 130381 | agatgtgata tttatgataa gaatagcaca gaggatggat ggggagcata tggaagtatg |
| 130441 | acattgtagt atgtagtatg atttcctata ttttgcatag aatggtatga aactaattca |
| 130501 | atagatcatg acaagttaag gatgcatatg gtaatcccca gtctggtgaa aaacagtaca |
| 130561 | aagagatatg ataacaagcc aatagaggaa ttaaaacaaa atattaaaaa atatttacta |
| 130621 | acccaaagga aggcaggaaa ggaggaaaag aggaataaaa agcacatgag acatgtagga |
| 130681 | taacaaaaca ggaactctaa atccaactat atcaataatg atgatggatg taaacataaa |
| 130741 | aatctaatca ctccaattac aaggcagagg tcacagtgta taaaaaggca agacccaact |
| 130801 | atatgctgcc tacaacaaat atactttaaa tacaaagaca gaagaataga aaacaaaaaa |
| 130861 | aatatgttct gcaaacacta agcatgagaa aactggaatg gctgtgacaa tatcgcacaa |
| 130921 | gatagacttt aagagtattt ctagaggtaa ggagggaagg agagacattt cataattata |
| 130981 | aaagagctaa tatatctgaa agacataaaa accatgaata tgtatgtgct taatgacaga |
| 131041 | ggtccacaat acacgacaca aacaaatgac atgtacatcc tgcccaaggg cagtttattc |
| 131101 | caaggatgta cagttgtttt aacatttgaa aacaaatcat tgtaatttac catattaaca |
| 131161 | aataaagaag aaaaaccata tcattctctc aatacatgga ccaaataagc atttgaaaac |
| 131221 | tcaataatca ttcacgataa aaactctcct aaaaaataga aatagaagag aattccctca |
| 131281 | atctgataaa agacatctgt ggaaaaccta tagcttacat catacttaaa gatgaaactt |
| 131341 | gaactctttt cctaatattg ggaaaacaca cagatgtctg ctctcaccat ttctatttga |
| 131401 | cattgtagtg gaggtcccag tcattataat atgacaagaa aaaaagtata aagattgaaa |
| 131461 | cagaaaaaag taaaagcatc cctattcaca gatgatagga ttatatttct atatagaaat |
| 131521 | ctattcctat tcttaattat atatggtaaa ttccattcat tatagggtta aaaaaatgtt |
| 131581 | aaatgcctaa ggcctttaca ctcaaaacga ctgcattgtt gagagaaatt aaagatgacc |
| 131641 | taaaaaacat aagttataac atattcagga attggaaaag tcaatattgg taagataata |
| 131701 | gttctctcca aattagctca tatatccagt gtaatcccta tcataatccc agcagaaatt |
| 131761 | gaaaggtgat tctaaaattt atggaacaat gaaaaggacc taaaatagcc aaaacaacct |
| 131821 | tgaaaaagaa caacgttgga acatcgcatg acttgatctt aaaggctgac taataagcta |
| 131881 | tggtaatcag gaccatgtgg gattggcata agaacttacc tattttttaa atctttgctc |
| 131941 | aaagaaccaa ttttactact ccattgctaa taaaacatgg gccttttaaa ggtcctgaat |
| 132001 | ggggtctatt ttatgctgtt attattatgt tattattatg atgcatttgt tgttgttgtt |
| 132061 | gttgttgttg agacagagtt ttgctcttgt cgcccaggct ggagggcaat ggcgcaatct |
| 132121 | ctgctcactg caacctctgc ctcccgggtt caagcaattc tcctgcctca gcctcctgag |
| 132181 | tagctgggat tacaggcgcc cgccaccatg cccaggctaa tttatttata tatatatatg |
| 132241 | tatatatatg tatgtgtgtg tgtgtgtgtg tatatatatg tgtatataca tatatgtgta |
| 132301 | tgtgtgtgtg tgtgtgtata tatatatata tatatttttt tttttttgag acggagtctc |
| 132361 | gctctgtcac ccaggatgga gtgcagtggc gccatctccg ctaactgcaa gctctgcctc |
| 132421 | ccaggttcac gccattctcc tgcctcagcc tcccgagtag ctgggactac aggtgcccac |
| 132481 | caccacgcct ggctaatttt ttgtattttt agtatagatg gggtttcacc gtgttagcca |
| 132541 | ggatggtctc gatctcctga cctcgtgatc tgcccgcctc aggctcccaa agtgctggga |
| 132601 | ttacaggcgt gagccaccgc gcccagccta atttttctat ttttagtaga gatgggtttc |
| 132661 | aacatgttgg ccaggctggt ctcgaattcc tgacctcaga taatccaccc gccacggcct |
| 132721 | cccaaagtgc tgggattaca ggcgtgagct accgcacccg gcctttatga tgcattttta |
| 132781 | ttattccttc agtagattgt gtgtttctcc ttgcatccag gcacttggtg ttatctaagt |
| 132841 | gtttatgtct tccatgtctg cactatgcta tatctgtagt ttgttttaaa tagtatctgt |
| 132901 | taaaacagcg accattatcc attgtagaca accagtcaca gtcctcatgg tgctgatgaa |
| 132961 | acacaaaagc cacaaagctt cttttcctca aataacctac ccgaggtgga gggctggctg |
| 133021 | gtttttgatg acacccattt tggtgaagac tcaggatgtg gggccacagg ttgcactgga |
| 133081 | cgagtctgtt tggctgccag tttcatcaga ctcacttgcc tcttgtgaaa tatttcctgg |
| 133141 | acatcatcca gcctctgcaa aactttctgg gctttggcct acagtaacaa aagcaaatca |
| 133201 | tgatgaacag gtctctctgt atacagcctg aggacacgaa gcattccctt tctcccacct |
| 133261 | tcccctactc cccccatccc tacctccctc aggtgacccc ctctgggcat cactatgcaa |
| 133321 | gtcgctgcag gtcctgccat gctccagaat tgggtatcta aggattagcc tctcctactt |
| 133381 | gagacccttg aggacaatga ctacatcctt tcactatggc gtctccagtg cctggaacac |
| 133441 | cctggcacag gagtttgaga ccagcctggt caacatggtg aaatcacatc tctactaaaa |
| 133501 | atacaaaaat tagccgggtg tggtggctgg cacctgtcat cccagctact tgagaagctg |
| 133561 | aggcaggaga atcgcttgaa cccaggaggc agaggttgca ctgagccgag atcatgccac |
| 133621 | tgcactccag cctgggcgac agagcgactc catctcagaa ataaacaaac aaacaaacaa |
| 133681 | ttagaataca atgtggcttg tgtcatgtta gacagagcaa agaattttgg cctgagtcca |
| 133741 | tggatggact tcagggcatt ccccagaatt acacaaacag ttagacaggc tggatataat |
| 133801 | ctgtttgttc ctccactgca tcttctcttc ctttctttgt gcccaggaag ctgattaaaa |
| 133861 | gaagctccta aaccagaggt gcagacaagg tgatgatgtg gctctcagag gaaggtgtgc |
| 133921 | tacctacaac cctgctggct tcatggaaga atatgcttct caaatgcaaa tccaatcaag |
| 133981 | ttatctccct gcttaaatcc aaactcgctc atgtggccca caaagcctac cttgcctgcc |
| 134041 | tctctctaac ctcacccaaa acacacttcc attgctctct aggttccagc accctgacct |
| 134101 | tttggtctgc actgtgccag gctccctcca gccctgaagc ctttgcacat gctgttcctc |
| 134161 | ctgtctggaa aggctctccc atcttgtcta ttcgttctcc aggctcaagt gactcttcct |
| 134221 | gagagccttc tctgacccca gtccaggtca agttcctctg tcacatgcct gaccctgcat |
| 134281 | ccctccttga cagcacttat atcagcttgt aacacatttt tgtgtgatta ttttgttaat |
| 134341 | gtcagcctct cccacaaact gtaagctcag tgagggatgg aagcatgcct atttttaatc |
| 134401 | atcatggtat cctcagcact cagcacagca catggtacat caggaatgct cataagcatt |
| 134461 | aatagagaaa tgactgattt gagtcagacg aagactcagt atgccaggac gttcaggccg |
| 134521 | agagtctaag aggccgccgg agggcttcgg aaccagtgag cccactcaat tcagcctgct |
| 134581 | cagtaccctg tgtgtacttt atcaggaaga aggtacagtc cctccctccc ctatccgttt |
| 134641 | cccaaccaca ggatcaaaat aacccggaag cattaccttt gcatcgaggg tgagcagcaa |
| 134701 | ctcaaactcg ttgtaaaact ccttggggct gagcaacggg tactccttga ctgtgcccag |
| 134761 | gaatgtcgca atgtcgttca aggcgatatc aaccccttct cgagactggc acttgtctac |
| 134821 | agcttgggaa gccaagaggt agattcctgc ctcacaccat tggctgacct tttggaaaga |
| 134881 | aacagtgccc tgtgcacttc gcctgaccac aactcagagc caccgtaatc ctcagcaggt |
| 134941 | ctgagcttgt aaggtgtcta caaggattcc cagacaatgg gaaattgtca tgggacggca |
| 135001 | tcgagtttaa ggggtctaac cactgtaaat tacactatga gggggacaaa cccaaaccag |
| 135061 | ctgatacctg tccacttctc agggaagtca ctcggtcagc aaaatgagtc tcttccatta |
| 135121 | acagtaattg ctacatctcc aggaggagac agctaaatat atatttaatt aaagacaggg |
| 135181 | tttcactctg tcacccaggc tggagtacag tagtgcaatt atggcttaat gcagcctgga |
| 135241 | gaggtctacc ccaggctcag gtgattcttc caccttagcc tcctgagtag ctgggactac |
| 135301 | agatgcatgc caccacacca ggctaatttt tccttccttc ctcccttcct tccttccttc |
| 135361 | cttccttcct ctttctttct ctctgtctct ctttctttct ttcctttttt tgagacaggt |
| 135421 | tttggccatg ttgccgaggc tgctctcgaa ctcctgggct caagcaatcc tcccacctcg |
| 135481 | gcctcccaaa gtgctgggat tacaggtgtg aggcactgca cccacccagc cttatagcta |
| 135541 | aatatctctt aggctggtct aaccatagag aaatgtaatc aatggctttt ggggtttacc |
| 135601 | tgctagtcca ctcttcttgc atccgaattg gttaccagaa cacagggtga ttaataagac |
| 135661 | attagacctg gcccttacat ttcctgggag aagggcatgt cccttttaac aaaaacacaa |
| 135721 | ctttcatctt tggattccca ggtgatccct atttgcattt actggcgttt gtctttcgta |
| 135781 | aggaagcaag aatgcaaagc tttgtaactc agcacagcga tactcaagag tttctggatg |
| 135841 | ctgggaggac attagatagg tgatgagttc taattattca atttttaaaa aagactcatt |
| 135901 | ttctttgctc ttttgaactc cactgagaca cttgaacaga aagacaggga gcccttatgc |
| 135961 | tacctgaaga gttatatgaa aagaagtttc cagtgccaat acctctgcat ttccatttac |
| 136021 | catgtgacct gaaaagcaat ataaagtaac tatacagaga aaaaagatca aaatgcaaca |
| 136081 | cgccaaagtg gttgtctttc taagtgggaa aatgataggc aacttaagtt tttgtcttat |
| 136141 | ttttttccta gattatttac agtgaatatg taccacattt agaataataa aaaatcataa |
| 136201 | agatatttca gaattaataa ttaccattat gtagggatgg gggatagtgg gatatgtacg |
| 136261 | tgcccatgag taaccttttt cttcctacat ttttggattt ccacaataat atgaactcat |
| 136321 | gctttgatat atgctgtgaa aactttctct ttacacatca tgcccagatc tgcaaaaaac |
| 136381 | attaggctgg gattttctgt agagttacta tggctacttg ctttctctct ctctctgtct |
| 136441 | ccctctctct ctctcaaaca cacatgcaca cacacacaca cacacacaca cacaccaggc |
| 136501 | ttataatctc tggagcaaaa atggagttgg gaaaacagca gctgatccca cagcgagctc |
| 136561 | agcacacaga gcaggctgca caccagcccc ttcggagcta cttgcttctg aaacaactat |
| 136621 | atttcattga cattaaaatc cctttagcaa tgggatgagg taagcgcctg ggtgggtgcc |
| 136681 | ccctgccatg caagaggaac agctgtgggg atggagcatg ctgactcatt gccgtcagcc |
| 136741 | acatgctgcc tgggaccagc tcacagggaa gactccagaa accgctgctc ctgctgactg |
| 136801 | ggaacatacc ctagaggtca cctccaaagg gcacccctct ccttgtgggc ccctggcatg |
| 136861 | cgcccttgct gtctatcctg tgaagccttg gtgtggaatc tggcaggcct gactttaatt |
| 136921 | ctggcttccc ctgtgtgcac catgtgtggt cttgggcagt catttacccc agggtttctt |
| 136981 | ggtgacatcc ctgcaccacc tgcaccagaa tcatttagac actttactaa aatactagtt |
| 137041 | cctgggcagt attccagacc tatcgacgct gagtcttcag ggcagtgcat aagggaatct |
| 137101 | gccatataac agctcactag gtgattctcg tgtgctacat tttgagaccc agcagagtat |
| 137161 | cagtttctgc ctctgcacaa tgaggccatt aataccttcc tcagaggttg tcttgaggat |
| 137221 | tataaataga tgacataatt atgttcctgg taggaatgca gtaggtgctc gatacatggt |
| 137281 | agggggtata aaatggttgt tattacttaa gcttctccaa aaagagtcaa aaccctttgc |
| 137341 | ctttatatga gggaagcact ctgcaaatat agtgcttggt tctctgtaca cctgaaaggg |
| 137401 | aatcaagaag tgttcccaca gggccaagcc aaaacacact cttaaagtcc ggctccacac |
| 137461 | agatagaaat gggcttcagt taactttaat gggatgtggt gggatggcca gagcactttt |
| 137521 | taccttcgtg ccaggtatac ttctacaccc caggatctgc acagttttta atgcctctcg |
| 137581 | aacatctttt cacataaagc actcaagcct ctcagcaatc acacaaggaa gggaggacga |
| 137641 | gtatgatgga agcttcttca cagaaaatct tgcagacatc atgttcttgg ctgtgttttg |
| 137701 | ctcaccttgt ccagctgtct atgaaactct aaggactttc ctaaaatgtc ccattttttc |
| 137761 | ttgtttccat tgatgaaatc gtcacagagg tgcctgagct ccacacaccg gggcctgatg |
| 137821 | gcatctgctg cataatggtg gctttggatg agctggtccc caaccagtgc cagcagcWgg |
| 137881 | gccttttcca ggggctcctg caaagtgaac acccacagcc aggcgtcaga agtcagagca |
| 137941 | tcatgaggct gagagctgcc tggtactgtg tgctccaagc ctggagggag gctgtggaga |
| 138001 | tgctaaccca ggatgaaggc tggagaacct gagaaagctc ggaatggttc caggcccagg |
| 138061 | aagttaagcc tggcccagag ttgtccacat gctgaacaac agcagtcatt cattcaaata |
| 138121 | cctgttgagt tctgggctag acatcgagga tatagtgtta acaagcccaa aattttcaga |
| 138181 | ttaatggaga attcagatat caactaaatt acacaaatcg ttaaattatt acaattgcaa |
| 138241 | taaactctag ggcagaaaag catggagtgc acataataag agattttgaa tacccctatt |
| 138301 | aactaaagga acaagaagag ggactgacac ctgtccttga ggagagtggg gggtgagctg |
| 138361 | aagttgaaac actggtgaaa attggatttg gacaactcaa aggtggtcat gacacttaag |
| 138421 | aaatatgctt gtctaggctg ggcgtggtgg ctcccacctg taatgccagc actttgggag |
| 138481 | gccgaggtgg gcggatcacc tgaggtcagg agttcaagac gcgcctgacc aacgtggaaa |
| 138541 | aaccccgtct ctactaaata caaaaattag atgggcgtgg tggtaggtgt ctgtaatccc |
| 138601 | aactactcag gaggctgagg caggagaatc gcttgaaccc gggaggcgga gattgcagtg |
| 138661 | aattgagatt gcgccactgc actccagcct gggtgacaga gcaagactcc atctcaaaaa |
| 138721 | aaaaaaaaaa aaaaaaaaga aatatgcttg tctaaaccct ttcccaaagc ctgtaagggc |
| 138781 | cctgtctaca ctgaccctgt ctgctcaatc acatttccta cctctttctg catgacactt |
| 138841 | cgctctaacc acacgtgcca agttttcctg tttgtttaag cattccttca ttcatccttc |
| 138901 | tcatattatt gagaacctgc tatttgccaa acactgtcat aggtgctggg agttcattgg |
| 138961 | taaagaaagc aggcaaaaac ccccacctca tgaaatgaag cttacatttt aatgggagac |
| 139021 | agaggcaata aacaagtgca tgtgtaggtc atatggtatc ttagacagtg atgagtatgt |
| 139081 | ggagaaaaat tagatggtgg ggagggcagg gagtgctgga gctagtattt taaatcggat |
| 139141 | ggtgagacag tctccctgag aaggagtact caagctatgc agaaaagact tcacatagaa |
| 139201 | agcctgagac taggccgggc tgtggtggct cacgcctgta atcctagcac tttgggaggc |
| 139261 | cgaggcaggc ggattgcctg agttcaggag ttggagacca gcctcggcaa cacagggaaa |
| 139321 | ccccatctct actaaaacaa aaaaaattag ccaggcatgg cagcatgcgc cgatagtccc |
| 139381 | agctactctg gaagctgagg caggagaatt gcttgaacct gggaagagga ggttgcagtg |
| 139441 | agccaagatc gtgccactgc actccagcct gggtgagagc taggctctgt ctccaaaaaa |
| 139501 | aaaaaaaaaa gagaaaagaa agcctgacac tacagcctta gaaagaaaga cccgatttta |
| 139561 | agattggccc tttgttggca tctaggaact tagatttttg ggaaggtttc ctccattccc |
| 139621 | tgatgtgaat ggttcatgat ccctgaactg tttgtgcaaa cagtatggtt tatggtgatt |
| 139681 | acttgctttt tcttctggga gtctgaaatc ttggtacttg ccagacagaa gctgctgaca |
| 139741 | tgaccagcca ccaataaaac ctctgggcat tgagtctcta atgagctccc ctggtagaca |
| 139801 | acatttcaca catgttgtca caacccacag acgggggcag gaagtatgtc ctgtgagact |
| 139861 | cccacaggag aggattctaa gaagcttgtg cctggtttcc tccagacttc acctcgcatg |
| 139921 | cctttctcct ttgctaattt tgcttagtac tttctcatct taatagatca taactctaat |
| 139981 | acagctacat gcagaatcct cctagtgaat cttcgaagaa atgtgtgcat ggtcttggga |
| 140041 | gcgctctgca tacaagctga gacctgaagt cagggacaag ccgtgcagta cttggctggg |
| 140101 | aggtgaaggc gtgtttgaca agtttgggta gcaggaagga ggccagtgtg gctgcagcag |
| 140161 | agggagcaag aaggaaatga gtggaagact aactagagga aagagggggt tccatagccg |
| 140221 | gggtgagagg aaagcccctg ggtttggagg gaggatctcg atcatgtttt tgaaaggacc |
| 140281 | actcccggta cagtgtccag aataagagga aatgcaggga gactcattaa ggggctgctg |
| 140341 | caggaatcca gccgagagat acggcttacg gctggtcacc aaactagctc ctgactcgac |
| 140401 | ccttctctgg attgttcttc cctgggtcat gcagagctga gttcctgggt cacctcctca |
| 140461 | cagcgcttcc ctggcgacta tatcttaagt cattctctac aacctattcg cccagtcacc |
| 140521 | taggccctct cccacacttt aattctttaa cagcattatc tctatctaaa aactattttc |
| 140581 | tttatttact ttaatgtaga attcctccct cctggctggg cgcggtggct cacaccagta |
| 140641 | atcccagcac tttgggaggc cgagacgggc agatcatgag gtcaggagat cgagaccatc |
| 140701 | ctggctaaca cggtgaaacc ccatctctac taaaaagaca aaaaattagc caggcatggt |
| 140761 | ggcacacacc tgtagtccca gctactcggg aggctgaggc aggagaatag cttgaacctg |
| 140821 | ggaggcggag gttgcagtaa gctgagattg tgccactgca ctccagcctg ggcaacagaa |
| 140881 | tgagactctg tctccaaaaa aaaaaaagaa ttcctccctc ctccactcac tcccaccaaa |
| 140941 | atgtaagttc catgagagca tttagtaggc atgcaataaa tatttgatca acaaaataaa |
| 141001 | tgaatgagcc aatgagctac atgatgcggt gacctgcttg gctggaactc tgtgaagcaa |
| 141061 | tcagcacaga tgactgccac attccaaaag gtctccagga gcagagagcc catgagcccc |
| 141121 | cttgccagcc acactaagca tccagtgcat cactgcactg ctttagctgt ttacagtaac |
| 141181 | acaaggagtc acggtaactc caggtgcgta catcttgccc ctctcttaac ttgcactggt |
| 141241 | ttatggtttc aacaatttcc aaaccccttt cctcttaccc tcccagtcat tcacgaagcc |
| 141301 | ccgcagggac cgtcgccttc ttaatgcctt tcaaattctg accccatctc tgccacctcc |
| 141361 | ttgtcactgt ctgctcaggc ctcatggctt ctcatgggga caactggtct ccctgctttt |
| 141421 | cttcaggctc tccttcaaat catgctccag actgctgaca aaggagcaat tccaacatat |
| 141481 | cttattgtgt ctacatcact cccaagcttc aagccctttc ctgggcgccc agggtcttca |
| 141541 | ggagcaagct taaactcttg agcaatgcac acggggaatt tcattatcta gctcatacct |
| 141601 | gggtgtgcct gacttaccaa gtcctcttcc acttcctcaa ctccattctc tgctgcaacc |
| 141661 | atactaatta tccacttatt gttccccaaa tattcaggca gcttcaggcc tccatccctt |
| 141721 | ctccacctgc tgtctgccta gaaagcccat ccttccctac tccccctcac ccatactcct |
| 141781 | ctggggccaa ctcgatctaa atcatatttc agacttcacc ttctccagaa aactttctct |
| 141841 | gtgcttctct tgatcagagc acctgcttca ttatgttgtg accacttatc tgctgcctgc |
| 141901 | gctatgccac agacacctat ggtccagaac cagctcttat tcaactgaaa acagtgcatg |
| 141961 | ctgcatctta ggtacttaca aataaatgct gcacaaaaca aagaggagta ctggtataga |
| 142021 | aacaagtgac acaaatcaag tagtggctag ttaaaaggac tgaggcaaaa atacccagaa |
| 142081 | cacttgttct aaacagcaaa ggcccaggtg cactggttac cacccctaca agatgactgg |
| 142141 | aatactcatt ccctgggcag tgattttttt tttttttttt tttttgagat ggaatcttgc |
| 142201 | tctgtagcta ggctggagtg cagtggcttg atctaggctc actgaaaatt ctgcctccgg |
| 142261 | ggttcaagcg attctcctcc ctcagcctcc cgagtagctg ggactatagg cgcccaccac |
| 142321 | tatgcctggc taattttttt tttttttaat ttttagcaga gatggggttt caccatgttg |
| 142381 | cccaggctgg tctcgaactc ctgagctcag gcaattcacc cgcctcggcc tcccaaagtg |
| 142441 | ctaggattac aggcgtgagc caccgcgcct ggtctgggca gtgatttttg actctttggt |
| 142501 | tcactgctgt gtcctcagaa cttaaacagt acctggcatg gtaggagatg aataaatatt |
| 142561 | tgcagaatag atgtcgaatg aatgacagac ctttcctagc actgctcagc taccgcagac |
| 142621 | tgaacctagc acatgttcca tagcccagaa tttatccctc tacctctgta atgttaataa |
| 142681 | caacagctaa catctactga gagcttaaca gatgccaggc actgttctga atgctttaca |
| 142741 | tttgcttatt tccttaactc ttcacaacaa accctttgat acaggtactg ttttaccaaa |
| 142801 | gggaagtgga gcacaatggc ccaaggtgac ccaggtagga aagagaagag ccaggcttca |
| 142861 | aacccaggca gtcttgctcc aggatctggc tcagaccaac atacacagag aaaaatggct |
| 142921 | gcacttgttc tgcctaaact tagagacctc atttggttgc tcgaacttgt ctgactttag |
| 142981 | tcaaatatag aaacatgaga cttggcatga aagtctctgt tgacactttt ttttttttta |
| 143041 | agtccagctg cctaatttat taagaacagg gcagaatttt ctgggtccag ggaaattcac |
| 143101 | cacaggatac ttccaaatca ccctgtggtg atcagagcct gcccttccct cagcatacag |
| 143161 | tatttcaccc ttcagaattt tccagaagac atttgagatg ttagagggaa gatgtgtgta |
| 143221 | acaggtccaa atgggtctag ggagaaacgc ccataaaggc aactgaggag gccgggcgca |
| 143281 | gtgggtcacg cctgtaatcc cagcactttg ggaagccaag gccggcggat cacaaggtca |
| 143341 | ggagttagag accagcctga ccaacatggt gaaaccccat ctctactaaa aatacaaaaa |
| 143401 | ttagccgggt gtggtggagg gtacctgtag tcccagctac ttgggaggct gagacagaag |
| 143461 | aatcgcttga acccaggagg cggaggttgc agtgagctga gattgtgcca ctgcactcca |
| 143521 | gcctgggcgg caagagtgaa actccgtctc aaaaaaaagg gcaactaagg agcaacccgg |
| 143581 | attcagccac cacatggctg gaaggtcact attacagaat tgaaaggaca acaatagttt |
| 143641 | ctgaccactg actactgacc accaaccata ttgcaagctg accacatgaa gtccctacca |
| 143701 | cacatctaac tcatttaatc ttcacaaggt ctctgtgagg caggaattat tacctctgct |
| 143761 | ttacagatga gggggaccat attcagaaag gctacgaaac gtgactaggg tcatacagct |
| 143821 | ggtacttgtt agagccaaga tttgaatcca tccctatccg acttccaaag cctgtgttct |
| 143881 | tgtcaccttc cagcccacct ggcctctaca catgtgctat gaaatcctcc tggtgcctgc |
| 143941 | aaagaaattt atggtgaaac ctccaaattg aagcttattt ttttagttta tatacctccc |
| 144001 | actcaggata aatagtcatc aaaatctcca aagtaaaaaa gaaagagtga gggaaaaaat |
| 144061 | ggctaaaatc ttctgatcac aagcccatct gggatctttc agatgctgaa aattccagaa |
| 144121 | ggctggatcg tcaagtttca gcttagaagt tgcaatcaga aatctttatt gaaataagtt |
| 144181 | tatttccccc cgaggactct gctggtataa cagatgagag atgagagtgc ctactaggga |
| 144241 | agaaagtggc gagaaagcga gtgatgctac tctatccggg aatctagaac aaacccacag |
| 144301 | aggatccaca gggatggagt gtgtgtatgg ggaggtgggc cagatgaaac aagaaaggca |
| 144361 | gaaagctaat ggttgttgaa attgaatgat ggacacttgg agttcattgt gccagtctat |
| 144421 | ctgcttctct gtgtgtttga aaatttcaca catgtacaca aagccatgac cctccaacaa |
| 144481 | gatgacatcc ttagatgtta atggcctggg ttccatgcct tcttagacac agcaccctta |
| 144541 | tggggagagg tcaaggctga aatcaggtgg ttcctccacc ctgtcctggt ctgttccctg |
| 144601 | ctggcccctc agcaagggtc tcagtacggc ccacagtcaa aaccctgcag caagttctgc |
| 144661 | ttttatagaa gaccttctgt aagtggttga aactactgtt tttcagaatc aggaaggagc |
| 144721 | attcaaaaga ctgctaaacc ctcaaacgag aacaagataa agaacaggct gaatcagcat |
| 144781 | gcgacaggag cctggttagt gagttgactg aggttacttg tgagaatgat gtcactgtgt |
| 144841 | ggaataagga agctggcaac accccagagc cagcgtgtgg ctgtgctggg agggagctgg |
| 144901 | ccccttagtg ccgagtaaag gacaaactcc aggcagatat ttgtcagatt gtggccagac |
| 144961 | acagtcttgt tcctttaaca ctttaaagga caaatggaaa gattagccaa gccctaatca |
| 145021 | tctgaagaag ccatgtgtct gagaagtctg agggaagata tattgaaaac gatctcagtt |
| 145081 | acgatgagac agctttgttt gatttacgca aaggcagaga cacaggtgta ctacatcctt |
| 145141 | caggaagcaa gtttagaaat aagatctgca agtcatagga tgtgacaggt ctgcaaatcc |
| 145201 | tgcaagtcac aggtcatcca cagtgggcta gtctagctgc ttcaagaatg ttgcccacga |
| 145261 | aaggtgggtc atgggttcac agagcattcc tgtgttccat aaccacaggt ttcatcccta |
| 145321 | actctggtca gcaacttttg aagtatatac caaggaccac aagttcaagg aagtgggcag |
| 145381 | aagaaagaat gtgatgcagt catgcagatc catcgccact attcaagaca cagcccagag |
| 145441 | cgccttccaa gggctttctg ttccactgag aataaaattc aaatcctcac aaaagatctc |
| 145501 | agggtgcaac aggactacct ggcctctgct catctctgtt cactcatttc ctcctggccc |
| 145561 | acctgctcta gcaatgttcc ttgaactcac caagctcatt tccactttag gacctttgca |
| 145621 | cctgcagctc gctcttgctt tgatactatt ccctaagtct ttgcatgatt acacagatca |
| 145681 | aacagatctc agaccaaagg tcgtcttctc aggcctttcc agaccattct agctaaagta |
| 145741 | gcatccccag tcactttcta ttatatcttg ttttcttttt tatatcactt gttactatct |
| 145801 | gatgttatta tttatttgat tgctttttag tgaactgtct ctctccagta gaatgtaagc |
| 145861 | tccatgaggg cagattctgg actgtctggc tcaccgctgc acccacggaa cctaggataa |
| 145921 | ggaatacctc ttgatgaata aataaccctc tggataggtc agtagctact ttcatatata |
| 145981 | tatgaaggca ttttttttga tgaggcactt tttcttaata tatgaataag caaaaaaata |
| 146041 | tagtatctat attcagtaaa aaattatttt gaaaaaacta aaatgatata aggttggggc |
| 146101 | tgtcctggag aatctaaggt atctagtcat catatatatg aaattcaact tttccattgt |
| 146161 | ttgataaatg ttcctgatga ctcatgtgtc tttagcacta gtttatctgt caaatggtca |
| 146221 | ctggtaagag aaagagcgct cgctgtgaag ggatgggatt cctgaatggg gatctggata |
| 146281 | cacagctggg tttgtgtgtt catgcatgtg cacaagcacc tgcagctcag aatctacaac |
| 146341 | caacagctct tagagtcagt gtggccttgg tatactttgt tttgtatcct ttaggggtgt |
| 146401 | gtgtctccaa ctttaacgta caagtcaatc acctggggat cttcttaaaa tgcagattcc |
| 146461 | aattccaggg tggggtctga gattttatat ttctaattag cgcttactca ggtgacatct |
| 146521 | gtgctgctgg ccagaggctc acactttggc aaatctctag tactgcaaaa ttgccgtctg |
| 146581 | agaaacatgc ctccagatta gctaagaaag gaagcgctaa ccacagggga cttggtcccc |
| 146641 | aagacccaaa aatatagaga acctaatttc aaacaagcag aaaggaactt ctctggaaag |
| 146701 | agacgactca gctttcttca tttgtaataa accaaatctg taaacactta aaatggaatc |
| 146761 | atttctaaat gctgacttgt gtgcggatct gggacactgt ttaattccag tatgggactg |
| 146821 | taaaatacat attcttttac aagtttcaca tttacaactc ttgggacatg agaaaggaga |
| 146881 | aagctttgcc aattctgtgt agcaatggaa ctaacatgga ccaatttccc ccatctaatt |
| 146941 | gtcttgctcc aggtataaaa ctagttgttt ttttttttct ggtttctcct ttgaaagttt |
| 147001 | cccatcctta ctaccaaccc cacaaccccc agccccctag tccaggctcc agggaggagc |
| 147061 | tcaagctgat gggtgagcct ctgatataat tgttctggtg agtgatgagc ctgttgtgac |
| 147121 | cttctcccac ttggctgctg accacgtgag agggcagggc tggtccagag tcctcacccg |
| 147181 | acctgagcgt ccaactgcaa ctgtatgttc cggcagcttt aataggactc tgtgctgccc |
| 147241 | gtattattca tagcagttgt taatgtgcct ctctccttgg gatcctgggg tgactgaagg |
| 147301 | gcaggactgg gcattcctgt gtcctttagc aaccttccaa tagcaatgac tggacatgat |
| 147361 | ccatcattgt ccacagcaac ctccctccca gtacacaaaa atgtcagaaa gcaaacctgg |
| 147421 | cttttttcct ccagtttttt gtgttcctta agaaKctgct ccacgtgcat cacgctgtct |
| 147481 | ccaatgcctg taaactctgc ttgctcttcc agcaaattat ccagggcaag cttagcctac |
| 147541 | aagaaacatt agaacagtcc agaagtaaac caacactcac tcacctgaag gtataggttt |
| 147601 | gttatgcttc aagaaaactc aaaattccaa aaaggattca agacttgtat atatctatat |
| 147661 | atgttaaaaa tttttttttt gcagctcact aactttcaaa |

Following is a KIAA0861 genomic nucleotide sequence that includes the KIAA0861 SNP “KIAA0861-AA” (SEQ ID NO: 276).

GTGCCTTTCTGAACCCTGTGACCCAGCAGCCTCCATCAACTCGTCCTACC
TGCCATGCACAGCTCCTCTGTGCCCCTGTACCTGAGCTCATGCTATTCCC
TCTGCCAGGATGCCCTTCTCCTTCTCCACCAGGAGAAGAACACTTGCCAG
TAAGACCCAGTTCTAATGTCACCCCTTCCTGACGGTATCAGGAAGAGTCA
[G/C]TGATGGTGTTTTATGCTCCCAGAGAATTTGCCACATTGTGTTGTG
ATTATTTTTCCACATCTGTCTCCCCCACTGGAATGAGAGCCTCACTCATC
TTCATACCTCCCTGGTCTCTACCTGGTGCCAGAACCATCCTCAGGGCAGG
GGAATGCTCAGGAAATAGATATTGAATAAAATAAGTGTATCCATCCATCC
ATCCA

Following is a first KIAA0861 cDNA sequence (SEQ ID NO: 2).

KIAA0861 Coding Sequence (cDNA) Corresponding to
SEQ ID NO: 2. (cDNA from positions 94 . . . 3189)
cgctgaattctaggggaggatggcgcccccatcatcacgttcccagagtt
ttcggggttcaaacacatcccagatgaagacttcctgaatgtcatgacct
acctgactagcatccccagtgtggaggctgccagcattggattcattgtt
gttatcgacagacgaagagacaagtggagctccgtaaaggcatccttgac
acgaatagctgtggcatttccaggaaacttacagctcatattcatccttc
gtccatctcgctttatccagaggacattcactgacattggcattaaatac
tatcgaaatgagtttaaaacgaaagtgccgatcatcatggtaaactctgt
ctctgaccttcacggctacatcgacaaaagccaactgacccgggaattag
gggggactttggaatatcgccacggtcagtgggtaaatcaccgcactgcc
atcgaaaactttgccttgaccttgaagaccactgcccagatgctgcagac
gtttgggtcctgcctggccacagcagagctgcccagaagcatgctatcca
cggaagaccttctcatgtcccacacaaggcagcgggacaagctgcaggat
gagctgaaattacttggaaagcaggggaccacattgctgtcatgcatcca
agaaccagcaaccaaatgtcccaacagcaaactcaatctcaaccaacttg
agaatgtaactaccatggaaaggttattagttcaactggatgaaacagaa
aaagcctttagtcacttttggtctgagcatcatctgaagcttaaccagtg
cctacaactacagcattttgagcacgatttttgtaaggctaagcttgccc
tggataatttgctggaagagcaagcagagtttacaggcattggagacagc
gtgatgcacgtggagcagattcttaaggaacacaaaaaactggaggaaaa
aagccaggagtccctggaaaaggcccagctgctggcactggttggggacc
agctcatccaaagccaccattatgcagcagatgccatcaggccccggtgt
gtggagctcaggcacctctgtgacgatttcatcaatggaaacaagaaaaa
atgggacattttaggaaagtccttagagtttcatagacagctggacaagg
tcagccaatggtgtgaggcaggaatctacctcttggcttcccaagctgta
gacaagtgccagtctcgagaaggggttgatatcgccttgaacgacattgc
gacattcctgggcacagtcaaggagtacccgttgctcagccccaaggagt
tttacaacgagtttgagttgctgctcaccctcgatgcaaaggccaaagcc
cagaaagttttgcagaggctggatgatgtccaggaaatatttcacaagag
gcaagtgagtctgatgaaactggcagccaaacagactcgtccagtgcaac
ctgtggccccacatcctgagtcttcaccaaaatgggtgtcatcaaaaacc
agccagccctccacctcggtccctctagctcgtcctctgagaacgtctga
ggaaccttatacggagacagagttgaactcccggggaaaggaagatgatg
agactaaatttgaagtcaagagtgaagaaatctttgaaagccatcatgaa
agggggaaccctgagctggagcagcaggccaggctcggagacctttcccc
ccgcaggcgcattatacgtgacttgcttgagactgaagagatttacataa
aagagattaaaagcataattgatggatatatcactccaatggattttatt
tggctaaagcatctaattccagatgttcttcagaataacaaggactttct
ctttgggaatattagagaactttacgaatttcacaacaggacttttctaa
aagagttggaaaagtgtgctgagaaccctgaacttctggcacattgcttt
ctcaagagaaaagaagatcttcagatatattttaaataccataagaatct
gccccgagctagggcaatctggcaagagtgtcaagactgcgcctactttg
gggtatgccagcgccaactggatcacaatctccctctttttaagtatctc
aaaggaccaagccagagacttataaaataccagatgctgttgaagggtct
gctggatttcgagtctcctgaagatatggagatagacccaggtgaactag
gaggctcggctaaggatgggccaaagagaaccaaagattcagcattctca
actgaactacaacaagctttggcagtgatagaggatttgatcaagtcctg
tgagttggctgtggacctagcagcagtgactgaatgtccggacgatattg
gaaaactaggcaagctgttgctgcacggccctttcagcgtctggacaatt
cacaaggatcgttataaaatgaaggatttgattcgatttaaacccagcca
gaggcaaatctacctatttgaaaggggaatagtgttctgtaagatacgaa
tggagcctggggaccagggattatctcctcattacagcttcaagaaggcc
atgaagctgatgacactttcaattcgccagcttggaagggggagccatag
aaagtttgagattgccagtcgaaatggacttgagaaatacatcctgcagg
cagcttcaaaagaaatcagagactgttggttttcagaaataagtaaatta
ttgatggaacaacaaaataatatcaaagaccaaggaaatccacagtttga
aatgagcacgagcaaaggcagtggagcaggatccggaccatggattaaaa
atatggaaagagctaccactagcaaggaagacccggcctccagcacagga
gggattaaaggctgctccagcagggagtttagctccatggacacctttga
agactgtgaaggcgcagaagacatggaaaaggagagcagtgctctgagtc
tcgcgggccttttccagtcggacgacagtcacgaaacctgttcctccaaa
tctgctttcctggagaggggagaaagcagccagggagaaaaagaagaacg
cgatgaggaggaaacggcgacccgcagcaccgaggaggagcgcgctgggg
cgtccacgggccggctggctcctgcgggggcgacggctggtttccaggcg
agggcgctgcgcccgaggacctccgcccaggagagctga

Following is a second KIAA0861 cDNA sequence (SEQ ID NO: 3).

ttgggcggagatgcctttaaaaaatcatccaccgcagcggtagaaacagt
tttgtttggctttatttatacggaatggtttttcagtgaaatgctgtctt
gcttaaaagaagagatgcctccccaggagctcacccggcgactggccaca
gtgatcactcatgtcgatgaaattatgcagcaggaagtcagacccctgat
ggcggtggagataatagaacaacttcacagacaatttgccattctttcag
gaggccgaggggaggatggcgcccccatcatcacgttcccagagttttcg
gggttcaaacacatcccagatgaagacttcctgaatgtcatgacctacct
gactagcatccccagtgtggaggctgccagcattggattcattgttgtta
tcgacagacgaagagacaagtggagctccgtaaaggcatccttgacacga
atagctgtggcatttccaggaaacttacagctcatattcatccttcgtcc
atctcgctttatccagaggacattcactgacattggcattaaatactatc
gaaatgagtttaaaacgaaagtgccgatcatcatggtaaactctgtctct
gaccttcacggctacatcgacaaaagccaactgacccgggaattaggggg
gactttggaatatcgccacggtcagtgggtaaatcaccgcactgccatcg
aaaactttgccttgaccttgaagaccactgcccagatgctgcagacgttt
gggtcctgcctggccacagcagagctgcccagaagcatgctatccacgga
agaccttctcatgtcccacacaaggcagcgggacaagctgcaggatgagc
tgaaattacttggaaagcaggggaccacattgctgtcatgcatccaagaa
ccagcaaccaaatgtcccaacagcaaactcaatctcaaccaacttgagaa
tgtaactaccatggaaaggttattagttcaactggatgaaacagaaaaag
cctttagtcacttttggtctgagcatcatctgaagcttaaccagtgccta
caactacagcattttgagcacgatttttgtaaggctaagcttgccctgga
taatttgctggaagagcaagcagagtttacaggcattggagacagcgtga
tgcacgtggagcagattcttaaggaacacaaaaaactggaggaaaaaagc
caggagcccctggaaaaggcccagctgctggcactggttggggaccagct
catccaaagccaccattatgcagcagatgccatcaggccccggtgtgtgg
agctcaggcacctctgtgacgatttcatcaatggaaacaagaaaaaatgg
gacattttaggaaagtccttagagtttcatagacagctggacaaggtcag
ccaatggtgtgaggcaggaatctacctcttggcttcccaagctgtagaca
agtgccagtctcgagaaggggttgatatcgccttgaacgacattgcgaca
ttcctgggcacagtcaaggagtacccgttgctcagccccaaggagtttta
caacgagtttgagttgctgctcaccctcgatgcaaaggccaaagcccaga
aagttttgcagaggctggatgatgtccaggaaatatttcacaagaggcaa
gtgagtctgatgaaactggcagccaaacagactcgtccagtgcaacctgt
ggccccacatcctgagtcttcaccaaaatgggtgtcatcaaaaaccagcc
agccctccacctcggtccctctagctcgtcctctgagaacgtctgaggaa
ccttatacggagacagagttgaactcccggggaaaggaagatgatgagac
taaatttgaagtcaagagtgaagaaatctttgaaagccatcatgaaaggg
ggaaccctgagctggagcagcaggccaggctcggagacctttccccccgc
aggcgcattatacgtgacttgcttgagactgaagagatttacataaaaga
gattaaaagcataattgatggatatatcactccaatggattttatttggc
taaagcatctaattccagatgttcttcagaataacaaggactttctcttt
gggaatattagagaactttacgaatttcacaacaggacttttctaaaaga
gttggaaaagtgtgctgagaaccctgaacttctggcacattgctttctca
agagaaaagaagatcttcagatatattttaaataccataagaatctgccc
cgagctagggcaatctggcaagagtgtcaagactgcgcctactttggggt
atgccagcgccaactggatcacaatctccctctttttaagtatctcaaag
gaccaagccagagacttataaaataccagatgctgttgaagggtctgctg
gatttcgagtctcctgaagatatggagatagacccaggtgaactaggagg
ctcggctaaggatgggccaaagagaaccaaagattcagcattctcaactg
aactacaacaagctttggcagtgatagaggatttgatcaagtcctgtgag
ttggctgtggacctagcagcagtgactgaatgtccggacgatattggaaa
actaggcaagctgttgctgcacggccctttcagcgtctggacaattcaca
aggatcgttataaaatgaaggatttgattcgatttaaacccagccagagg
caaatctacctatttgaaaggggaatagtgttctgtaagatacgaatgga
gcctggggaccagggattatctcctcattacagcttcaagaagaccatga
agctgatgacactttcaattcgccagcttggaagggggagccatagaaag
tttgagattgccagtcgaaatggacttgagaaatacatcctgcaggcagc
ttcaaaagaaatcagagactgttggttttcagaaataagtaaattattga
tggaacaacaaaataatatcaaagaccaaggaaatccacagtttgaaatg
agcacgagcaaaggcagtggagcaggatccggaccatggattaaaaatat
ggaaagagctaccactagcaaggaagacccggcctccagcacaggaggga
ttaaaggctgctccagcagggagtttagctccatggacacctttgaagac
tgtgaaggcgcagaagacatggaaaaggagagcagtgctctgagtctcgc
gggccttttccagtcggacgacagtcacgaaacctgttcctccaaatctg
ctttcctggagaggggagaaagcagccagggagaaaaagaagaacgcgat
gaggaggaaacggcgacccgcagcaccgaggaggagcgcgctggggcgtc
cacgggccggctggctcctgcgggggcgacggctggtttccaggcgaggg
cgctgcgcccgaggacctccgcccaggagagctgacctccctgcggacgc
ccccgctcctcggctccagagcgcccgcattcccgggagaggcggtgtgg
gggcccgggccctgcccagctacgcagaaagcagccggagcctcggcggc
ggcagaaaggggacaaccagggcctcctccgaggagcccgaggggtgtcc
tgggtgcgcgcctagctccgcacgggggacctcggagctgctctaaggcg
cctgcagaggcgagcagagcccgcagcccacgccttctcgaccgcgcact
tcgacattcggagccgggcaattctttgctgcgtgggcttctctgtgctc
ccacggtaggatgtttagtagcacccctggcctctacccactaggtgcca
ggaatgcgccaccccatcctccaccccgcccccaaatcgtgacaatcaaa
aatgcctgcagacacgcccgcatttctccagggcggggtgggaatcggtt
gagagccgcttagcccgagccttggaggagccggagccgctcaaacccgg
cgggggccgcagactgggagctcccggtccgcctcccagcatccctgcga
gcgttcatggggtgttcgtgttagtgccaagattgcttcgttgtagagag
agttcgttccaagttactttctgaggtattttatgtatcgatttattagt
ttttaaatgagttttgttagtttcagttgtattttatttttagttttatt
agttgattttcatttctttgtagctttctggttatttaattttatagttt
cagttactggtttatagttactttattttcaaagttatttagttgttcat
tttcagttatttatatgtagttgttttgttttaggagttacagatgttca
aattaatttgcttggaatttatttatttatttatttatttatttttcgag
acggagtctcgctctgtcgcccaggctggagtgcagtggtgtgatctcgg
ctcactgcaaaccgcctcccgggttcacgccattctcctgcctcagcctc
ctgagtagctgggactacaggcgcccgccaccacgcccggctaatttttt
atttggatttttagtagagacggggtttccccgtgttagccaggatggtc
tcgatctcctgacctcgtgatccccctgcctcggcctcccaaagtgctgg
gattacaggcgtgagccaccgcgcccagccggaatttatttttaattatc
tttacaattattatctgagttatttaccttcatagttatttcactttatc
ttattggagtttttgagttatttatttttacagtaacttatttgactatt
aaacactcactggaagttcatg (4772 bp)

Following is a first KIAA0861 amino acid sequence (SEQ ID NO: 4).

MTYLTSIPSVEAASIGFIVVIDRRRDKWSSVKASLTRIAVAFPGNLQLIF
ILRPSRFIQRTFTDIGIKYYRNEFKTKVPIIMVNSVSDLHGYIDKSQLTR
ELGGTLEYRHGQWVNHRTAIENFALTLKTTAQMLQTFGSCLATAELPRSM
LSTEDLLMSHTRQRDKLQDELKLLGKQGTTLLSCIQEPATKCPNSKLNLN
QLENVTTMERLLVQLDETEKAFSHFWSEHHLKLNQCLQLQHFEHDFCKAK
LALDNLLEEQAEFTGTGDSVMHVEQTLKEHKKLEEKSQESLEKAQLLALV
GDQLTQSHHYAADATRPRCVELRHLCDDFTNGNKKKWDTLGKSLEFHRQL
DKVSQWCEAGTYLLASQAVDKCQSREGVDTALNDTATFLGTVKEYPLLSP
KEFYNEFELLLTLDAKAKAQKVLQRLDDVQETFHKRQVSLMKLAAKQTRP
VQPVAPHPESSPKWVSSKTSQPSTSVPLARPLRTSEEPYTETELNSRGKE
DDETKFEVKSEETFESHHERGNPELEQQARLGDLSPRRRTTRDLLETEET
YTKETKSTTDGYTTPMDFTWLKHLTPDVLQNNKDFLFGNTRELYEFHNRT
FLKELEKCAENPELLAHCFLKRKEDLQTYFKYHKNLPRARATWQECQDCA
YFGVCQRQLDHNLPLFKYLKGPSQRLTKYQMLLKGLLDFESPEDMEIDPG
ELGGSAKDGPKRTKDSAFSTELQQALAVIEDLIKSCELAVDLAAVTECPD
DIGKLGKLLLHGPFSVWTIHKDRYKMKDLIRFKPSQRQIYLFERGIVFCK
IRMEPGDQGLSPHYSFKKAMKLMTLSIRQLGRGSHRKFEIASRNGLEKYI
LQAASKEIRDCWFSEISKLLMEQQNNIKDQGNPQFEMSTSKGSGAGSGPW
IKNMERATTSKEDPASSTGGIKGCSSREFSSMDTFEDCEGAEDMEKESSA
LSLAGLFQSDDSHETCSSKSAFLERGESSQGEKEERDEEETATRSTEEER
AGASTGRLAPAGATAGFQARALRPRTSAQES (1031 aa)

Following is a second KIAA0861 amino acid sequence (SEQ ID NO: 5).

MLSCLKEEMPPQELTRRIATVITHVDEIMQQEVRPLMAVEIIEQLHRQFA
ILSGGRGEDGAPIITFPEFSGFKHIPDEDFLNVMTYLTSTPSVEAASTGF
TVVTDRRRDKWSSVKASLTRTAVAFPGNLQLTFTLRPSRFTQRTFTDTGT
KYYRNEFKTKVPTTMVNSVSDLHGYTDKSQLTRELGGTLEYRHGQWVNHR
TATENFALTLKTTAQMLQTFGSCLATAELPRSMLSTEDLLMSHTRQRDKL
QDELKLLGKQGTTLLSCTQEPATKCPNSKLNLNQLENVTTMERLLVQLDE
TEKAFSHFWSEHHLKLNQCLQLQHFEHDFCKAKLALDNLLEEQAEFTGTG
DSVMHVEQTLKEHKKLEEKSQEPLEKAQLLALVGDQLTQSHHYAADATRP
RCVELRHLCDDFTNGNKKKWDTLGKSLEFHRQLDKVSQWCEAGTYLLASQ
AVDKCQSREGVDTALNDTATFLGTVKEYPLLSPKEFYNEFELLLTLDAKA
KAQKVLQRLDDVQETFHKRQVSLMKLAAKQTRPVQPVAPHPESSPKWVSS
KTSQPSTSVPLARPLRTSEEPYTETELNSRGKEDDETKFEVKSEETFESH
HERGNPELEQQARLGDLSPRRRTTRDLLETEETYTKETKSTTDGYTTPMD
FTWLKHLTPDVLQNNKDFLFGNTRELYEFHNRTFLKELEKCAENPELLAH
CFLKRKEDLQTYFKYHKNLPRARATWQECQDCAYFGVCQRQLDHNLPLFK
YLKGPSQRLTKYQMLLKGLLDFESPEDMEIDPGELGGSAKDGPKRTKDSA
FSTELQQALAVIEDLIKSCELAVDLAAVTECPDDIGKLGKLLLHGPFSVW
TIHKDRYKMKDLIRFKPSQRQIYLFERGIVFCKIRMEPGDQGLSPHYSFK
KTMKLMTLSIRQLGRGSHRKFEIASRNGLEKYILQAASKEIRDCWFSEIS
KLLMEQQNNIKDQGNPQFEMSTSKGSGAGSGPWIKNMERATTSKEDPASS
TGGIKGCSSREFSSMDTFEDCEGAEDMEKESSALSLAGLFQSDDSHETCS
SKSAFLERGESSQGEKEERDEEETATRSTEEERAGASTGRLAPAGATAGF
QARALRPRTSAQES (1114 aa)

Following is an amino acid sequence alignment between KIAA0861 domain and SEC14 domain sequences (SEQ ID NOS 277-280, respectively in order of appearance).

| | | |
|---|---|---|
| Sequence 1: KIAA0861 | 1031 aa | Sequences (1:2) Aligned. Score: 38 |
| Sequence 2: Hs_DBS_GNEF | 1108 aa | Sequences (1:3) Aligned. Score: 37 |
| Sequence 3: Mm_DBS_GNEF | 1149 aa | Sequences (1:4) Aligned. Score: 40 |
| Sequence 4: Rn_DBS_GNEF | 937 aa | Sequences (2:3) Aligned. Score: 83 |
| | | Sequences (2:4) Aligned. Score: 87 |
| | | Sequences (3:4) Aligned. Score: 96 |

| | | |
|---|---|---|
| KIAA0861 | ---------MTYLTSIPSVEAASIGFIVVIDRRRKDWSSVKASLTRIAVAFPGNLQLIPI | 51 |
| Hs_DBS_GNEF | IPDKEFQNVMTYLTSIPSLQDAGIGFILVIDRRRDKWTSVKASVLRIAASFPANLQLVLV | 180 |
| Mm_DBS_GNEF | IPDKEFQNVMTYLTSIPSLQDAGIGFILVIDRPQDKSTSVKASVLRIAASFPANLQLVLV | 165 |
| Rn_DBS_GNEF | -------------------QDAGIGFILVIDRPQDKWTSVKASVLRIAASFPANLQLVLV | 41 |
| | : *.****:*****:***:*****: ***.:**.****::: | |
| KIAA0861 | LRPSRFIQRTFTDIGIKYYRNEFKTKVPIIMVNSVSDLHGYIDKSQLTRELGGTLEYRHG | 111 |
| Hs_DBS_GNEF | LRPTGFFQRTLSDIAFKFNRDDFKMKVPVIMLSSVPDLHGYIDKSQLTEDLGGTLDYCHS | 240 |
| Mm_DBS_GNEF | LRPTGFFQRTLSDIAFKFNRDEFKMKVPVMMLSSVPELHGYIDKSQLTEDLGGTLDYCHS | 225 |
| Rn_DBS_GNEF | LRPTGFFQRTLSDIAFKFNRDEFKMKVPVMMLSSVPELHGYIDKSQLTEDLGGYLDYCHS | 101 |
| | ***: *:***::**.:*: *::** ***::*:.**.:***********.:*****:* *. | |

Following is an amino acid sequence alignment between KIAA0861 domain and SPEC domain sequences (SEQ ID NOS 281-284, respectively in order of appearance).

| | | |
|---|---|---|
| KIAA0861 | KLLGKQGTTLLSCIQEPATKCPNSKLNLNQLENVTTMERLLVQLDETEKAFSHFWSEHHL | 231 |
| Hs_DBS_GNEF | RLALKEGHSVLESLRELQAEGSEPSVNQDQLDNQATVQRLLAQLNETEAAFDEFWAKHQQ | 360 |
| Mm_DBS_GNEF | QLALKEGNSILESLREPLAESAAHSVNQDQLDNQATVQPLLAQLNETEAAFDEFWAKHQQ | 345 |
| Rn_DBS_GNEF | QLALTEGNSILESLREPLAESIVHSVNQDQLDNQATVKRLLTQLNETEAAFDEFWAKHQQ | 221 |
| | :* .:* ::*..::* :: .:* :**:* :*::***.**:*** **..**::*: | |
| KIAA0861 | KLNQCLQLQHFEHDFCKAKLALDNLLEEQAEFTGIGDSVMHVEQILKEHKKLEEKSQESL | 291 |
| Hs_DBS_GNEF | KLEQCLQLRHFEQGFREVKAILDAASQKIATFTDIGNSLAHVEHLLRDLASFEEKSGVAV | 420 |
| Mm_DBS_GNEF | KLEQCLQLRHFEQGFREVKTTLDSMSQKIAAFTDVGNSLAHVQHLLKDLTAFEEKSSVAV | 405 |
| Rn_DBS_GNEF | KLEQCLQLRHFEQGFREVKTALDSMSQKIAAFTDVGNSLAHVQHLLKDLTTFEEKSSVAV | 281 |
| | **:*****:***:.* : * ** :: * **.:*:*: **:::*:: :**** :: | |
| KIAA0861 | EKAQLLALVGDQLIQSHHYAADAIRPRCVELRHLCDDFINGNKKKWDILGKSLEFHRQLD | 351 |
| Hs_DBS_GNEF | ERARALSLDGEQLIGNKHYAVSDIRPKCQELRHLCDQFSAEIARRRGLLSKSLELSRRLE | 480 |
| Mm_DBS_GNEF | DKARALSLEGQQLIENRHYAVDSIHPKCEELQHLCDHFASEVTRRRGLLSKSLELHSLLE | 465 |
| Rn_DBS_GNEF | -TSQSPVLEGQQLIENRHYAVDISHPKCEELQHLCDHFASEVTRRRDLLSKSLELHSLLE | 340 |
| | :: * *:*** .:***.*:*:*:* **:****.* :: .:*.****:* *: | |
| KIAA0861 | KVSQWCEAGIYLLASQAVDKCQSRESVDIALNDIATFLGRVKEYPLLSPKEFYNEFELLL | 411 |
| Hs_DBS_GNEF | TSMKWCDEGIYLLASQPVDKCQSQDGAEAALQEIEKFLETGAENKIQELNAIYKSYESIL | 540 |
| Mm_DBS_GNEF | TSMKWSDEGIFLLASQPVDKCQSQDGAEASFQEIEKFLETGAENKIQELNEIYKEYECIL | 525 |
| Rn_DBS_GNEF | TSMKWSDEGIFLLASQPVDKCQSQDGAEAALQEIEKFLETGAENKIQELNKIYKEYECIL | 400 |
| | . :*.: **:*****.******::*.: ::::* .** * * : . : :*:*:* :* | |
| KIAA0861 | TLDAKAKAQKVLQRLDDVQEIFHKRQVSLMKLAAKQTRPVQPVAPHPESSPKWVSSKTSQ | 471 |
| Hs_DBS_GNEF | NQDLMEHVRKVFQKQASMEEVFHRRQASLKKLAARQTRPVQPVAPRPEAL-----AKSPC | 595 |
| Mm_DBS_GNEF | NQDLLEHVQKVFQKQESTEEMFHRRQASLKKLAAKQTRPVQPVAPRPEAL-----TKSPS | 580 |
| Rn_DBS_GNEF | NQDLLEHVQKVFQKQESTERMFHRRQASLKKLAAKQTRPVQPVAPRPEAL-----TKSPS | 455 |
| | . * :.:**:*: . :*:**:**.** ****:**********:**: :*:. | |

Following is an amino acid sequence alignment between KIAA0861 domain and RhoGEF domain sequences (SEQ ID NOS 285-288, respectively in order of appearance).

| | | |
|---|---|---|
| KIAA0861 | GDLSPRRRIIRDLLETEEIYIKEIKSIIDGYITPMDFIWLKHLIPDVLQNNKDFLFGNIR | 591 |
| Hs_DBS_GNEF | AIL--RRHVMSELLDTERAYVEELLCVLEGYAAEMDNPLMAHLLSTGLHNKKDVLFGNME | 701 |
| Mm_DBS_GNEF | AIL--RRHVMNELLDTERAYVEELLCVLEGYAAEMDNPLMAHLISTGLQNKKNILFGNME | 687 |
| Rn_DBS_GNEF | AIL--RRHVMNELLDTERAYVEELLCVLEGYAAEMDNPLMAHLISTGLQNKKNILFGNME | 562 |
| | . * **::: :**:**. *::*: .:::** : ** : **:. *:*:*:.****:. | |
| KIAA0861 | ELYEFHNRTFLKELEKCAENPELLAHCFLKRKEDLQIYFKYHKNLPRARAIWQECQDCAY | 651 |
| Hs_DBS_GNEF | EIYHFHNRIFLRELENYTDCPELVGRCPLERMEDFQIYEKYCQNKPRSESLWRQCSDCPF | 761 |
| Mm_DBS_GNEF | EIYHPHNRNIPAGVESCIDCPELVGRCFLERMEEFQIYEKYCQNKPRSESLWRQCSDCFF | 747 |
| Rn_DBS_GNEF | EIYHFHNRIFLRELESCIDCPELVGRCFLERMEEFQIYEKYCQNKPRSESLWRQCSDCPF | 622 |
| | *:*.**** : :*. : ***:.:***:* *::*** ** :* **:.::*::*.**.: | |
| KIAA0861 | FGVCQRQLDHNLPLFKYLKGPSQRLIKYQMLLKGLLDFESPEDMEIDGGELGGSAKDGPK | 711 |
| Hs_DBS_GNEF | FQECQRKLDHKLSLDSYLLKPVQRITKYQLLLKEML-----------------------K | 798 |
| Mm_DBS_GNEF | FQECQKKLDHKLSLDSYLLKPVQRITKYQLLLKEML-----------------------K | 784 |
| Rn_DBS_GNEF | FQECQKKLDHKLSLDSYLLKPVQRITKYQLLLKEML-----------------------K | 659 |
| | * **::***:*.* .** * **: ***:*** :* | |
| KIAA0861 | RTKDSAFSTELQQALAVIEDLIKSCELAVDLAAVTECPDDIGKLGKLLLHGPFSVWTIHK | 771 |
| Hs_DBS_GNEF | YSRNCEGAEDLQEALSSILGILKAVNDSMHLIAITGYDGNLGDLGKLLMQGSFSVWTDHK | 858 |
| Mm_DBS_GNEF | YSKHCEGAEDLQEALSSILGILKAVNDSMHLIAITGYDGNLGDLGKLLMQGSFSVWTDHK | 844 |
| Rn_DBS_GNEF | YSKHCEGAEDLQEALSSILGILKAVNDSMHLIAITGYDGNLGDLGKLLMQGSFSVWTDHK | 719 |
| | **.. : :**:**: * .::*: : ::.* *:* .::*.*****::*.***** ** | |

Following is an amino acid sequence alignment between KIAA0861 domain and PH domain sequences (SEQ ID NOS 289-292, respectively in order of appearance).

| | | |
|---|---|---|
| KIAA0861 | DRY-KMDKLIRFKPSQRQIYLFERGIVFCKIRMEPGD-QGLSPHYSFKKAMKLMTLSIRQ | 829 |
| Hs_DBS_GNEF | RGHTKVKELARFKPMQRHLFLHEKAVLFCKKREENGEGYEKAPSYSYKQSLNMAAVGITE | 918 |
| Mm_DBS_GNEF | KGHTKVKELARFKPMQRHLFLHEKAVLFCKKREENGEGYEKAPSYSYKQSLNMTAVGITE | 904 |
| Rn_DBS_GNEF | KGHTKVKELARFKPMQRHLFLHEKAVLFCKKREENGEGYEKAPSYSYKQSLNMTAVGITE | 779 |
| | : *:*:* **** **:::*.*:.::*** * * *: :* **:*::::: ::.* : | |
| KIAA0861 | LGRGSHRKFEIASRNGLEKYILQAASKEIRDCWFSEISKLLMEQQNNIKDQGNPQ-FEMS | 888 |
| Hs_DBS_GNEF | NVKGDAKKFEIWYNAREEVYIVQAPTPEIKAAWVNEIRKVLTSQLQACREASQHRALEQS | 978 |
| Mm_DBS_GNEF | NVKGDTKKFEIWYNAREEVYIIQAPTPEIKAAWVNEIRKVLTSQLQACREASQHRALEQS | 964 |
| Rn_DBS_GNEF | NVKGDTKKFEIWYNAREEVYIIQAPTPEIKAAWVNEIRKVLTSQLQACREASQHRALEQS | 839 |
| | :*. :**** . * **:**.: **: .*..** *:* .* : :: .: : :* * | |

Modifications may be made to the foregoing without departing from the basic aspects of the invention. Although the invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims which follow. All publications or patent documents cited in this specification are incorporated herein by reference as if each such publication or document was specifically and individually indicated to be incorporated herein by reference.

Citation of the above publications or documents is not intended as an admission that any of the foregoing is pertinent prior art, nor does it constitute any admission as to the contents or date of these publications or documents. U.S. patents, documents and other publications referenced herein are hereby incorporated by reference.

1-85. (canceled)

86. A method for identifying a subject at risk of breast cancer, which comprises detecting the presence or absence of one or more polymorphic variations associated with breast cancer in a nucleotide sequence set forth in SEQ ID NO: 1, a substantially identical sequence thereof or complement of the foregoing, in a sample from a subject, whereby the presence of a polymorphic variant associated with breast cancer is indicative of the subject being at risk of breast cancer.

87. The method of claim 86, wherein the one or more polymorphic variations are selected from the group consisting of rs3811728, rs3811729, rs602646, rs488277, rs1629673, rs670232, rs575326, rs575386, rs684846, rs471365, rs496251, rs831246, rs831247, rs512071, rs1502761, rs681516, rs683302, rs619424, rs620722, rs529055, rs664010, rs678454, rs2653845, rs472795, rs507079, rs534333, rs535298, rs536213, rs831245, rs639690, rs684174, rs571761, rs1983421, rs4630966, rs2314415, rs6788196, rs2103062, rs9827084, rs9864865, rs6804951, rs6770548, rs1403452, rs7609994, rs9838250, rs9863404, rs903950, rs6787284, rs2017340, rs2001449, rs1317288, rs7635891, rs10704581, rs11371910, rs10937118, rs7642053, rs3821522, rs2029926, rs1390831, rs7643890, rs11925606, rs9826325, rs6800429, rs6803368, rs1353566, rs2272115, rs2272116, rs3732603, rs940055, rs2314730, rs2030578, rs2049280, rs3732602, rs2293203, rs7639705, and position 13507 of SEQ ID NO: 1.

88. The method of claim 86, which further comprises obtaining the nucleic acid sample from the subject.

89. The method of claim 86, wherein a polymorphic variation is detected at one or more positions in a region spanning positions 14647 to 48849 in SEQ ID NO: 1.

90. The method of claim 86, wherein a polymorphic variation is rs4630966.

91. The method of claim 86, wherein a polymorphic variation is rs9827084.

92. The method of claim 86, wherein a polymorphic variation is rs9864865.

93. The method of claim 86, wherein a polymorphic variation is rs6804951.

94. The method of claim 86, wherein a polymorphic variation is rs6770548, rs1403452 and rs2001449.

95. The method of claim 86, wherein one or more polymorphic variations are detected at one or more positions in linkage disequilibrium with a polymorphic variation at one or more positions selected from the group consisting of rs3811728, rs3811729, rs602646, rs488277, rs1629673, rs670232, rs575326, rs575386, rs684846, rs471365, rs496251, rs831246, rs831247, rs512071, rs1502761, rs681516, rs683302, rs619424, rs620722, rs529055, rs664010, rs678454, rs2653845, rs472795, rs507079, rs534333, rs535298, rs536213, rs831245, rs639690, rs684174, rs571761, rs1983421, rs4630966, rs2314415, rs6788196, rs2103062, rs9827084, rs9864865, rs6804951, rs6770548, rs1403452, rs7609994, rs9838250, rs9863404, rs903950, rs6787284, rs2017340, rs2001449, rs1317288, rs7635891, rs10704581, rs11371910, rs10937118, rs7642053, rs3821522, rs2029926, rs1390831, rs7643890, rs11925606, rs9826325, rs6800429, rs6803368, rs1353566, rs2272115, rs2272116, rs3732603, rs940055, rs2314730, rs2030578, rs2049280, rs3732602, rs2293203, rs7639705, and position 13507 of SEQ ID NO: 1.

96. The method of claim 86, wherein detecting the presence or absence of the one or more polymorphic variations comprises:
hybridizing an oligonucleotide to the nucleic acid sample, wherein the oligonucleotide is complementary to a nucleotide sequence in the nucleic acid and hybridizes to a region adjacent to the polymorphic variation;
extending the oligonucleotide in the presence of one or more nucleotides, yielding extension products; and
detecting the presence or absence of a polymorphic variation in the extension products.

97. The method of claim 86, wherein the subject is a human.

98. A method of genotyping a nucleic acid which comprises determining the nucleotide corresponding to position 13507 of SEQ ID NO: 1 in the nucleic acid.

99. An isolated nucleic acid which comprises a cytosine at position 13507 of SEQ ID NO: 1, or a cytosine at a position corresponding to position 13507 of SEQ ID NO: 1 in a substantially identical nucleic acid.

100. An oligonucleotide comprising a nucleotide sequence complementary to a portion of the nucleotide sequence of claim 99, wherein the 3′ end of the oligonucleotide is adjacent to a polymorphic variation.

101. A microarray comprising an isolated nucleic acid of claim 99 linked to a solid support.

102. An isolated polypeptide encoded by the isolated nucleic acid sequence of claim 99.

103. A method of targeting information for preventing or treating breast cancer to a subject in need thereof, which comprises detecting the presence or absence of one or more polymorphic variations associated with breast cancer in SEQ ID NO: 1, a substantially identical nucleotide sequence thereof or complement of the foregoing in a nucleic acid sample from a subject, and
directing information for preventing or treating breast cancer to a subject in need thereof based upon the presence or absence of the one or more polymorphic variations in the nucleic acid sample.

104. The method of claim 103, wherein the polymorphic variation is detected in a nucleotide sequence selected from the group consisting of:
(a) a nucleotide sequence in SEQ ID NO: 1, 2 or 3;
(b) a nucleotide sequence which encodes a polypeptide encoded by a nucleotide sequence in SEQ ID NO: 1, 2 or 3;
(c) a nucleotide sequence which encodes a polypeptide that is 90% or more identical to the amino acid sequence encoded by a nucleotide sequence in SEQ ID NO: 1, 2 or 3;
(d) a fragment of a nucleotide sequence of (a), (b), or (c) comprising the polymorphic variation.

105. The method of claim 103, wherein the one or more polymorphic variations are detected at one or more positions corresponding to a position selected from the group consisting of rs3811728, rs3811729, rs602646, rs488277, rs1629673, rs670232, rs575326, rs575386, rs684846, rs471365, rs496251, rs831246, rs831247, rs512071, rs1502761, rs681516, rs683302, rs619424, rs620722, rs529055, rs664010, rs678454, rs2653845, rs472795, rs507079, rs534333, rs535298, rs536213, rs831245, rs639690, rs684174, rs571761, rs1983421, rs4630966, rs2314415, rs6788196, rs2103062, rs9827084, rs9864865, rs6804951, rs6770548, rs1403452, rs7609994, rs9838250, rs9863404, rs903950, rs6787284, rs2017340, rs2001449, rs1317288, rs7635891, rs10704581, rs11371910, rs10937118, rs7642053, rs3821522, rs2029926, rs1390831, rs7643890, rs11925606, rs9826325, rs6800429, rs6803368, rs1353566, rs2272115, rs2272116, rs3732603, rs940055, rs2314730, rs2030578, rs2049280, rs3732602, rs2293203, rs7639705, and position 13507 of SEQ ID NO: 1.

106. The method of claim 103, wherein the information comprises a description of a breast cancer detection procedure, a chemotherapeutic treatment, a surgical treatment, a radiation treatment, a preventative treatment of breast cancer, and combinations of the foregoing.
